(19) United States
(12) Patent Application Publication
(10) Pub. No.: US 2026/0234737 A1
(43) Pub. Date: Aug. 13, 2026

(54) SINGLE NUCLEOTIDE POLYMORPHISM LIQUID CHIP FOR MUNG BEAN, MOLECULAR MARKER COMBINATION, AND APPLICATION THEREOF

(71) Applicant: Jiangsu Academy of Agricultural Sciences, Nanjing (CN)

(72) Inventors: Xingxing Yuan, Nanjing (CN); Jinyang Liu, Nanjing (CN); Xin Chen, Nanjing (CN); Yun Lin, Nanjing (CN); Shicong Li, Zhengzhou (CN); Ranran Wu, Nanjing (CN); Qiang Yan, Nanjing (CN); Jingbin Chen, Nanjing (CN); Na Yuan, Nanjing (CN); Dan Gong, Nanjing (CN); Chenchen Xue, Nanjing (CN); Xi Zhang, Nanjing (CN); Yixiang Pu, Nanjing (CN); Yan Wu, Nanjing (CN)

(21) Appl. No.: 19/365,185

(22) Filed: Oct. 22, 2025

(30) Foreign Application Priority Data
Feb. 8, 2025 (CN) 202510140466.X

(51) Int. Cl. C12Q 1/6895

(52) U.S. Cl. CPC C12Q 1/6895; C12Q 2600/13; C12Q 2600/156

(57) ABSTRACT

A mung bean single nucleotide polymorphism (SNP) liquid chip, a molecular marker combination and an application thereof are provided, and relate to the technical field of plant molecular genetic breeding. The SNP molecular marker combination includes 20526 SNP molecular markers. A mung bean SNP chip is further provided, including at least one of a probe and a primer configured to detect the mung bean SNP molecular marker combination. The mung bean SNP chip can rapidly and effectively track mung bean genetic materials, facilitating the acceleration of breeding processes for high-yield, early-maturing, high-quality, and disease-resistant mung bean varieties, while improving the efficiency of selecting superior new varieties. The SNP molecular marker combination represents a first mung bean breeding chip on the market, featuring numerous important functional loci related to yield, quality, and disease resistance, extensive genetic diversity, more uniform chromosome coverage, greater chip flexibility, higher detection rates, and better repeatability.

### CROSS-REFERENCE TO RELATED APPLICATION

This application claims priority to Chinese Patent Application No. 202510140466.X, filed on Feb. 8, 2025, which is herein incorporated by reference in its entirety.

### TECHNICAL FIELD

The disclosure relates to the technical field of plant molecular genetic breeding, and more particularly to a single nucleotide polymorphism (SNP) liquid chip (also referred to as suspension array) for mung bean, a molecular marker combination, and an application thereof.

### BACKGROUND

Mung bean (Vigna radiata L.), as a member of the Fabaceae family and the genus Vigna, has 11 chromosomes. As a functional food ingredient, mung bean seeds are rich in protein, essential amino acids, carbohydrates, dietary fiber, and bioactive compounds. With rising living standards and increasing demand for healthy diets, the nutritional value of the mung bean has gained recognition, leading to a growing demand.

The annual demand for the mung bean in China exceeds 900,000 tons, while the production of the mung bean in China is around 600,000 tons, requiring approximately 33% to be imported. In scientific research, accelerating the breeding of high-quality, high-yield, and early-maturing mung bean varieties, conducting joint research efforts, and improving the adaptability, high-yield potential, and quality of the mung bean varieties are main research directions in mung bean breeding. However, current molecular breeding research on the mung bean primarily focuses on a few qualitative trait loci, lacking an effective molecular breeding system for complex quantitative traits such as yield and quality, which are controlled by multiple genes.

The mung bean is native to Asia and is widely cultivated in temperate and subtropical regions. Major producing countries in Asia include India, China, and Thailand, with expanding cultivation in the United States and Australia. The mung bean is highly nutritious, with seed protein content ranging from 19.5% to 33.1%, significantly higher than cereal crops-2.3 times that of wheat flour, 3.0 times that of cornmeal, 3.2 times that of rice, and 2.7 times that of millet. Moreover, the mung bean is a highly stress-resistant crop with a short growth period, flexible sowing time, broad adaptability, benefits for land use and soil fertility, and relatively high economic benefits. However, the molecular breeding research on the mung bean remains scarce, particularly for complex quantitative traits like yield and quality. Currently, there is no report of a genome-wide SNP genotyping chip for the mung bean, further restricting its application in the mung bean scientific research and molecular breeding.

The deoxyribonucleic acid (DNA) molecular marker technology is a method that utilizes DNA sequence differences among individuals to identify genetic variation. Currently, there are four main types of DNA markers. The first type mainly includes: the restriction fragment length polymorphism (RFLP), the variable number tandem repeats (VNTR), and the single-strand conformation polymorphism (SSCP), etc. The second type mainly includes: the randomly amplified polymorphic DNA (RAPD), the simple sequence repeats (SSR), the sequence-tagged sites (STS), the expressed sequence tags (EST), and the sequence-characterized amplified regions (SCAR). The third type includes: the amplified fragment length polymorphism (AFLP) and the cleaved amplified polymorphic sequences (CAPS). The fourth type is the SNP-based DNA marker, primarily the single-nucleotide polymorphism (SNP). Compared with morphological, biochemical, and cytological markers, the DNA markers offer distinct advantages: most DNA markers are codominant, facilitating selection for recessive traits; the DNA markers also provide abundant genomic variation, offering a large number of markers; and their detection methods are simple and rapid. With advances in molecular biology, the DNA molecular markers have been widely applied in constructing genetic linkage maps, gene mapping, breeding, phylogenetic studies, gene-bank construction, and gene cloning in both plants and animals.

The SNP refers to the DNA sequence polymorphism caused by a single nucleotide variation in the genome. Any nucleotide in genomic DNA can exist as one of four bases (A, T, G, C). Typically, a specific genomic locus in a species exhibits only two alternative bases, with one base changing into another base through either a transition or a transversion, and the transition generally occurs more frequently than transversion. Thus, the SNP makers are usually biallelic and are widely distributed throughout the genome. Modern high-throughput SNP technologies fall into two categories: those based on next-generation sequencing (NGS) and those based on SNP genotyping arrays. New-type high-throughput molecular marker technologies based on the SNP fall into two main classes: one is a high-throughput molecular marker technology based on the NGS, and the other is a high-throughput molecular marker technology based on the DNA chip.

Based on SNP genotypes, the mung bean can be subjected to genetic-diversity analysis, genome-wide association studies, fine-scale quantitative trait locus (QTL) mapping, molecular-evolution analysis, marker-assisted breeding, and genomic selection etc., which will play a pivotal role in allele pyramiding and in broadening the crop's genetic base.

At present, there is no genome-wide SNP genotyping chip for the mung bean available, creating a critical gap in the era of molecular breeding that limits scientific research and molecular breeding applications for the mung bean in China.

### SUMMARY

In view of the foregoing, a first purpose of the disclosure is to provide a mung bean SNP molecular marker combination. The mung bean SNP molecular marker combination includes: 20526 SNP molecular markers as shown in Table 2, and a position and allelic information of each of the SNP molecular markers are in a format of chromosome (chr): physical position: reference genotype/variant allele genotype (ref/alt).

The disclosure utilizes the high-throughput DNA sequencing technology to conduct whole-genome resequencing on 613 mung bean samples sourced from various countries and regions. The analysis uses the Sulv 1 genome (submitted GenBank assembly: GCA_050395885.1) as the reference genome, with a burrows-wheeler-alignment (BWA) software for alignment and a genome analysis toolkit (GATK)-3.8 software for variant detection to obtain raw variant data from the 613 samples. The raw variant data is filtered through an inherent screening strategy process (referring to FIG. 1) to obtain a total of 4,048,862 high-quality variant sites. Based on customized chip design principles, 20,526 core SNP sites are selected from the high-quality variant sites.

A second purpose of the disclosure is to provide a mung bean SNP chip (also referred to as green core No. 1). The mung bean SNP chip includes: at least one of a probe and a primer configured to detect the mung bean SNP molecular marker combination.

The data for the green core No. 1 chip of the disclosure originates from the resequencing results of the 613 mung bean samples, the vast majority of which are core germplasms from China, possess extensive representativeness, and hold significant importance in mung bean genomics researches in China. The reference genome for the green core No. 1 chip is the SuLvl genome.

The probe configured to detect the mung bean SNP molecular marker combination is single-stranded DNA synthesized based on the 20K mung bean SNP molecular markers (i.e., the total of 20,526 SNP molecular markers).

A third purpose of the disclosure is to provide a kit. The kit includes at least one of a probe and a primer configured to detect the mung bean SNP molecular marker combination.

The disclosure further aims to provide an application method of the mung bean SNP molecular marker combination, the mung bean SNP chip, or the kit, including: applying the mung bean SNP molecular marker combination, the mung bean SNP chip, or the kit in mung bean variety identification.

The disclosure further aims to provide an application method of the mung bean SNP molecular marker combination, the mung bean SNP chip, or the kit, including: applying the mung bean SNP molecular marker combination, the mung bean SNP chip, or the kit in detecting a mung bean breeding material resistant to yellow mosaic disease.

The disclosure further aims to provide an application method of the mung bean SNP molecular marker combination, the mung bean SNP chip, or the kit, including: applying the mung bean SNP molecular marker combination, the mung bean SNP chip, or the kit in mung bean genome-wide association analysis.

The disclosure further aims to provide an application method of the mung bean SNP molecular marker combination, the mung bean SNP chip, or the kit, including: applying the mung bean SNP molecular marker combination, the mung bean SNP chip, or the kit in molecular marker assisted breeding.

The disclosure further aims to provide an application method of the mung bean SNP molecular marker combination, the mung bean SNP chip, or the kit, including: applying the mung bean SNP molecular marker combination, the mung bean SNP chip, or the kit in DNA fingerprint analysis of mung bean germplasm resources.

The disclosure further aims to provide an application method of the mung bean SNP molecular marker combination, the mung bean SNP chip, or the kit, including: applying the mung bean SNP molecular marker combination, the mung bean SNP chip, or the kit in mung bean genomic selection breeding.

The disclosure further aims to provide an application method of the mung bean SNP molecular marker combination, the mung bean SNP chip, or the kit, including: applying the mung bean SNP molecular marker combination, the mung bean SNP chip, or the kit in one of the following applications:
(1) mung bean parentage identification,
(2) mung bean population structure analysis,
(3) mung bean genetic diversity analysis,
(4) mung bean variety authenticity testing, and
(5) mung bean cultivar genotyping identification.

The disclosure at least has the following beneficial effects.

(1) Uniform distribution: The average distance is approximately 23 kilobytes (kb), and the coverage of SNP sites with the minor allele frequency (MAF)>0.1 reaches 99.4% within a 60K interval.

(2) High representativeness: The information is from the resequencing data of 613 representative mung bean core germplasms from China and internationally.

(3) Strong functionality: The high proportion of SNPs are large-effect sites of annotated genes, covering important gene-related sites, domestication selection intervals, and SNP sites unique to wild and cultivated mung bean subgroups.

(4) Good practicability: published important trait-related markers are basically covered, and important traits such as flavones, amino acids, vitexin, a plant height, a hundred-seed weight, disease resistance, and drought and waterlogging tolerance are highlighted.

(5) Support for site filling: the disclosure has an ultra-high-density site filling rate.

(6) Upgradable chip: The chip can be upgraded by continuously increasing the number of significant quantitative trait nucleotides (QTN)/quantitative trait loci (QTL) and closely linked sites based on additional phenotypic data and the full genome sequence after filling.

The solution of the disclosure is the first mung bean breeding chip on the market, featuring numerous important yield and quality and disease resistance functional sites, extensive genetic diversity, more uniform coverage of chromosomes, greater chip flexibility, higher detection rate, and better repeatability. The provided SNP molecular marker combination for mung bean genotyping and the mung bean whole-genome chip have uniform distribution, high representativeness, strong functionality, and good practicability, and support site filling. The chip can be upgraded based on additional phenotypic data to fill in the number of closely linked sites, offering significant economic value and application prospects. The genetic material of the mung bean can be quickly and effectively tracked, facilitating the breeding process of high-yield, early-maturing, high-quality, and disease-resistant mung beans, and improving the efficiency of selecting excellent new varieties.

### BRIEF DESCRIPTION OF DRAWINGS

FIG. 1 illustrates a basic flowchart for site selection of a green core No. 1 chip of the disclosure; where QTL represents quantitative trait locus mapping, GWAS represents a genome-wide association study, mGWAS represents a metabolite genome-wide association study, MAF represents a minor allele frequency, and SNP represents a single nucleotide polymorphism.

FIGS. 2A-2D illustrate distribution maps of the 20K mung bean liquid-phase gene chip sites across the whole genome of the disclosure; where in FIG. 2A, more than 19,700 representative variant sites covering the genome are selected; in FIG. 2B, the chip covers over 300 significant sites related to metabolite traits; in FIG. 2C, the chip covers more than 10 new sites related to yield traits, such as significant QTN sites for the number of pods per plant shown in figure; and in FIG. 2D, the chip covers functional genes/QTN mapping that have been reported in the literature.

FIG. 3 illustrates a SNP site density of the green core No. 1 chip of the disclosure, where a number of sites represents a quantity of SNPs.

FIG. 4 illustrates a MAF distribution of the SNP sites of the disclosure.

FIG. 5 illustrates an analysis result of functional sites for the green core No. 1 chip.

FIGS. 6A-6B illustrate population structure analysis diagrams of the green core No. 1 chip on 892 multi-parent advanced generation intercross (MAGIC) materials. FIG. 6A illustrates MAGIC population structure analysis, and FIG. 6B illustrates a MAGIC population structure display diagram.

FIGS. 7A-7B illustrate a Manhattan plot based on generalized linear model (GLM) for resistance to yellow mosaic disease and a validation result. FIG. 6A illustrates the Manhattan plot for resistance to yellow mosaic disease, and FIG. 6B illustrates a validation plot for the kompetitive allele specific polymerase chain reaction (KASP) molecular marker developed at Chr4-14132960.

FIG. 8 illustrates a GLM-based Manhattan plot for initial flowering time.

FIG. 9 illustrates a GLM-based Manhattan plot for hundred-seed weight.

FIG. 10 illustrates a diagram of phenotypes and predictions for various traits

### DETAILED DESCRIPTION OF EMBODIMENTS

A clear and complete description of the technical solutions in embodiments of the disclosure will be provided below. Apparently, the described embodiments are only a part of the embodiments of the disclosure, and not all of them. Based on the embodiments of the disclosure, all other embodiments obtained by those skilled in the art without creative labor are within the scope of protection of the disclosure.

In addition, the technical solutions between various embodiments of the disclosure can be combined with each other, but they must be based on the premise that it can be implemented. When the combination of the technical solutions is contradictory or impossible to implement, it should be considered that this combination of the technical solutions does not exist and is not within the scope of protection required by the disclosure.

Specific examples are provided below to illustrate the proposed solutions of the disclosure.

### Embodiment 1: SNP Site Selection

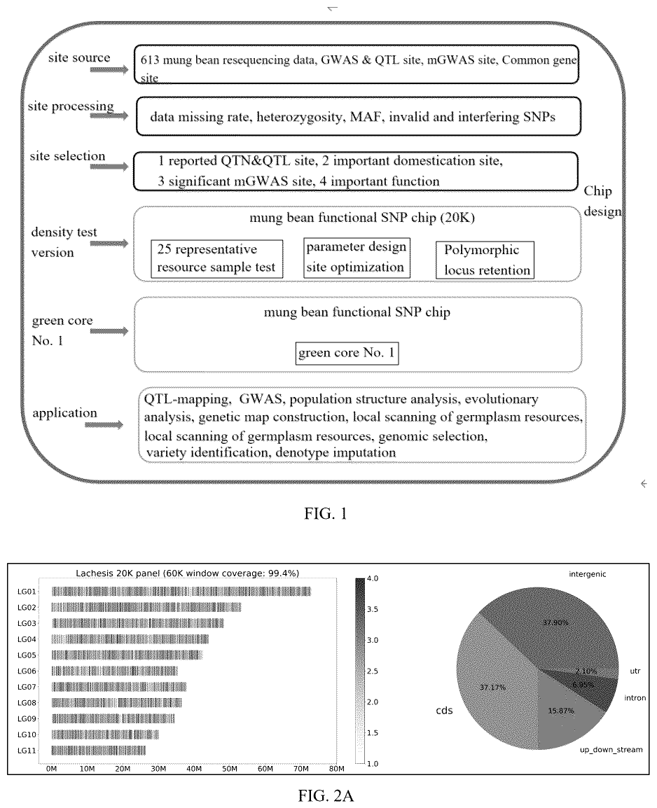

The SNP site selection process is shown in FIG. 1.

### (1) Initial Dataset is Collected.

The SNP marker information for the green core No. 1 chip originates from 613 mung bean germplasm resources provided by the Economic Crop Research Institute of Jiangsu Academy of Agricultural Sciences, including 414 core germplasm resources and 199 mini-core germplasm resources. These resources cover cultivated varieties, local varieties, and wild varieties from the Northeast, the Huang-Huai-Hai region, and the southern ecological zones, and international germplasms, forming a natural population with rich genotypic and phenotypic data. Whole-genome sequencing is performed using the NovaSeq platform (Illumina Inc.), achieving a 10× genome coverage for each sample. Reads with low quality and adapter contamination are excluded to obtain clean data. All the clean data are mapped to the Sulv1 genome using the BWA software, and then polymerase chain reaction (PCR) duplicates are removed, followed by variant calling using the GATK software, to obtain a total of 3,068,512 SNP variant sites.

Meanwhile, the SNP marker information for the green core No. 1 chip also originates from sites related to QTL, GWAS, domestication-related genes, intraspecific subgroup differences, desirable traits, terminators, alternative splicing, and non-synonymous mutation sites, as summarized in Table 1.

TABLE 1
Sources of SNP marker information for green core No. 1

| | Actual |
|---|---|
| SNP type (Tiling order) | content |
| Excellent QTL sites, GWAS sites (1) | 0.5K |

QTL sites
GWAS sites
Genes
Terminator/alternative splicing/non-synonymous mutation sites

| | |
|---|---|
| Significant mGWAS sites (2, 3) | 2K |

Intra-species and inter-species subgroup difference sites
Selection interval (domestication) sites

| | |
|---|---|
| Important genes (VIP), terminator/alternative splicing/non- | 17.5K |
| synonymous mutation sites (4) | |
| Total | 20K |

### Initial Data Quality Control is Performed.

Assessment: (1) Quality control: a sample call rate of 90%, a site call rate of 90%, a MAF>0.05, a sample heterozygosity rate being less than 20%, and Hardy-Weinberg equilibrium at 0.00001; (2) Non-repetitive samples; the above quality control is completed using a Plink software (an open source tool software for genetic data analysis), ultimately obtaining a set of 80,216 sites for subsequent selection of sites for the green core No. 1 chip. The chip site selection is based on custom site selection criteria to screen and evaluate the above data: (1) tiling order 1=excellent QTL sites, GWAS sites; (2) tiling order 2=interspecific and intraspecific subgroup differentiation domestication sites; (3) tiling order 3=significant mGWAS sites, sites significantly associated with metabolites; (4) tiling order 4=important genes (VIP), terminator/alternative splicing/non-synonymous mutation sites. Further comprehensive consideration of the sites is made according to the priority of tiling order, retaining the principle of MAF≥0.01, sites without polymorphism are deleted, and after deleting interfering sites in SNP flanking sequences, the sliding window selection is performed on the sequences. Other factors to consider include: preference for non-A/T, G/C sites; uniform genome coverage; site evaluation scores≥0.4; probe sequence redundancy <99%; a missing rate below 3%; whether it is a single copy; not located in transposable elements or repetitive regions.

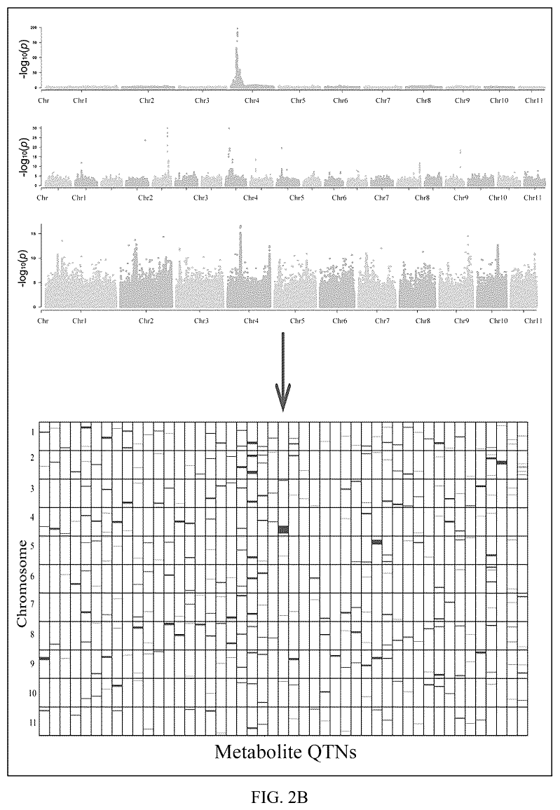

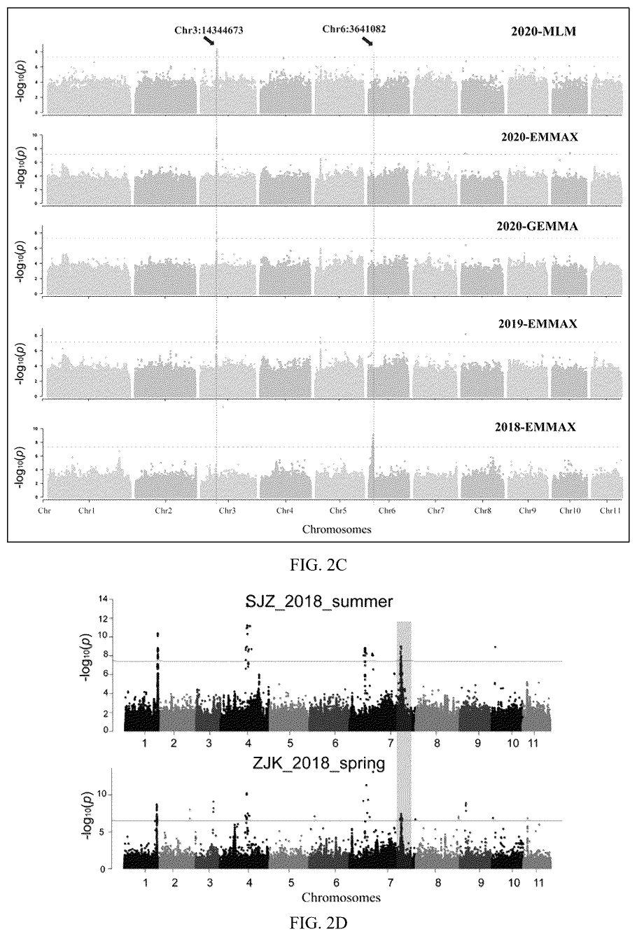

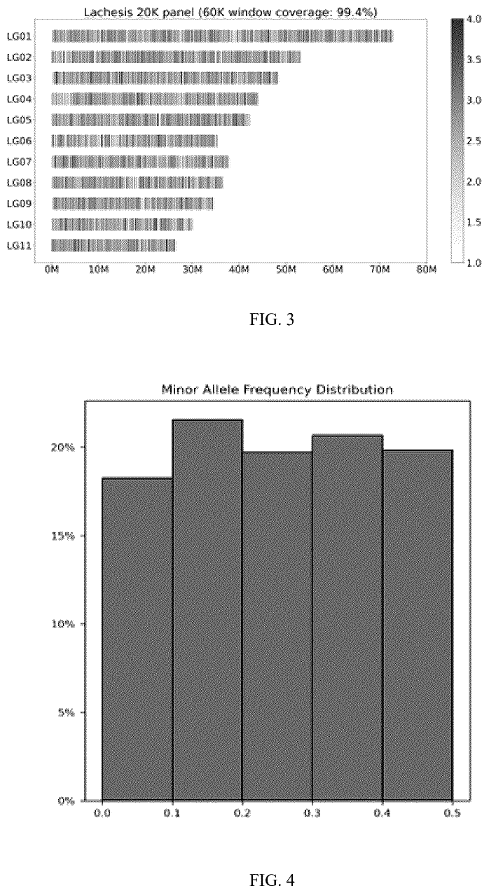

The set of variant sites for the green core No. 1 chip is constructed according to the above principles, and a total of 20,526 molecular markers (mung bean 20K) are finally selected. Their distribution on the mung bean genome is shown in FIGS. 2A-3, and the MAF values are shown in FIG. 4. It can be seen that the average distance between the molecular markers provided by the disclosure is about 23 kb, and the coverage of SNP sites with the MAF>0.1 reaches 99.4% within a 60K interval. The information of the 20,526 SNP molecular markers is shown in Table 2 below, which is the mung bean SNP molecular marker combination consisting of 20,526 SNP molecules in the disclosure.

TABLE 2
Positional information of SNP sites for
the mung bean 20K liquid-phase gene chip.

| | physical | |
|---|---|---|
| chr | position | ref/alt |
| 1 | 18318 | G/A |
| 1 | 27454 | G/C |
| 1 | 31246 | G/T |
| 1 | 57857 | G/A |
| 1 | 63121 | T/C |
| 1 | 91429 | C/T |
| 1 | 118834 | T/C |
| 1 | 136260 | T/C |
| 1 | 149104 | G/A |
| 1 | 173787 | G/A |
| 1 | 174865 | G/A |
| 1 | 192747 | A/T |
| 1 | 206950 | C/T |
| 1 | 248258 | C/T |
| 1 | 249670 | C/A |
| 1 | 317702 | G/A |
| 1 | 320028 | C/T |
| 1 | 333149 | T/C |
| 1 | 378584 | C/A |
| 1 | 383369 | A/G |
| 1 | 420035 | C/T |
| 1 | 421486 | T/C |
| 1 | 450701 | C/T |
| 1 | 502593 | G/A |
| 1 | 505562 | G/C |
| 1 | 529503 | G/A |
| 1 | 532636 | A/G |
| 1 | 540173 | A/G |
| 1 | 592576 | G/A |
| 1 | 595471 | G/A |
| 1 | 613701 | G/C |
| 1 | 625764 | C/T |
| 1 | 659299 | A/T |
| 1 | 664083 | C/T |
| 1 | 707993 | A/G |
| 1 | 741656 | G/A |
| 1 | 751019 | G/A |
| 1 | 790881 | C/T |
| 1 | 814153 | C/T |
| 1 | 853636 | G/A |
| 1 | 855326 | A/C |
| 1 | 877219 | C/T |
| 1 | 927240 | G/A |
| 1 | 939659 | C/T |
| 1 | 972298 | C/T |
| 1 | 992762 | T/C |
| 1 | 1021468 | G/T |
| 1 | 1052950 | A/G |
| 1 | 1062352 | A/C |
| 1 | 1084159 | T/C |
| 1 | 1098848 | C/G |
| 1 | 1110573 | G/A |
| 1 | 1130451 | T/C |
| 1 | 1156389 | A/C |
| 1 | 1165896 | C/T |
| 1 | 1175392 | A/T |
| 1 | 1186269 | C/G |
| 1 | 1224645 | T/C |
| 1 | 1254690 | C/T |
| 1 | 1267621 | T/C |
| 1 | 1270013 | A/G |
| 1 | 1304208 | G/A |
| 1 | 1307234 | C/T |
| 1 | 1340673 | A/C |
| 1 | 1360329 | G/C |
| 1 | 1405182 | T/G |
| 1 | 1418968 | G/A |
| 1 | 1449653 | C/T |
| 1 | 1455663 | T/C |
| 1 | 1479466 | A/G |
| 1 | 1524744 | A/G |
| 1 | 1535884 | G/T |
| 1 | 1545797 | T/C |
| 1 | 1560142 | G/A |
| 1 | 1580189 | C/T |
| 1 | 1592007 | C/G |
| 1 | 1639314 | C/T |
| 1 | 1644145 | A/G |
| 1 | 1653306 | G/A |
| 1 | 1658075 | G/T |
| 1 | 1699638 | G/A |
| 1 | 1726258 | A/G |
| 1 | 1756847 | T/C |
| 1 | 1763535 | C/T |
| 1 | 1773348 | G/C |
| 1 | 1783526 | G/T |
| 1 | 1802451 | C/G |
| 1 | 1850087 | C/T |
| 1 | 1858586 | G/A |
| 1 | 1877421 | G/C |
| 1 | 1888199 | T/C |
| 1 | 1890544 | A/C |
| 1 | 1919637 | C/G |
| 1 | 1921318 | C/T |
| 1 | 1926104 | T/A |
| 1 | 1955580 | A/C |
| 1 | 1968611 | C/T |
| 1 | 2002698 | G/C |
| 1 | 2025693 | G/A |
| 1 | 2064090 | T/A |
| 1 | 2071762 | C/T |
| 1 | 2086416 | T/G |
| 1 | 2113219 | T/A |
| 1 | 2122008 | T/G |
| 1 | 2149589 | G/A |
| 1 | 2180635 | A/G |
| 1 | 2207743 | C/T |
| 1 | 2220851 | C/T |
| 1 | 2267584 | A/T |
| 1 | 2297447 | T/A |
| 1 | 2334797 | T/G |
| 1 | 2345885 | A/G |
| 1 | 2378609 | C/T |
| 1 | 2385648 | C/T |
| 1 | 2415483 | A/C |
| 1 | 2457591 | C/T |
| 1 | 2469394 | G/A |
| 1 | 2490942 | T/G |
| 1 | 2501100 | G/A |
| 1 | 2526392 | C/T |
| 1 | 2529205 | C/T |
| 1 | 2550564 | G/C |
| 1 | 2552546 | A/G |
| 1 | 2593869 | G/C |
| 1 | 2610364 | T/A |
| 1 | 2645141 | T/C |
| 1 | 2658083 | T/C |
| 1 | 2689361 | T/G |
| 1 | 2694410 | T/G |
| 1 | 2718805 | A/T |
| 1 | 2731065 | G/A |
| 1 | 2750040 | C/T |
| 1 | 2768403 | C/T |
| 1 | 2782981 | T/A |
| 1 | 2804821 | A/C |
| 1 | 2808741 | G/A |
| 1 | 2841590 | C/T |
| 1 | 2865195 | T/C |
| 1 | 2888826 | A/T |
| 1 | 2918029 | G/C |
| 1 | 2940042 | A/G |
| 1 | 2946762 | T/C |
| 1 | 2972834 | T/C |
| 1 | 2986356 | C/T |
| 1 | 3014071 | T/A |
| 1 | 3016166 | T/C |
| 1 | 3030464 | A/T |
| 1 | 3043773 | C/A |
| 1 | 3085658 | G/T |
| 1 | 3114006 | G/T |
| 1 | 3149948 | C/A |
| 1 | 3175142 | A/T |
| 1 | 3182003 | C/T |
| 1 | 3205256 | G/T |
| 1 | 3210471 | C/T |
| 1 | 3241601 | C/T |
| 1 | 3247201 | G/T |
| 1 | 3275598 | T/G |
| 1 | 3302129 | T/G |
| 1 | 3311970 | T/C |
| 1 | 3332943 | G/A |
| 1 | 3339189 | A/G |
| 1 | 3364606 | C/T |
| 1 | 3373704 | G/A |
| 1 | 3413020 | A/G |
| 1 | 3427318 | A/G |
| 1 | 3443422 | G/A |
| 1 | 3451908 | C/T |
| 1 | 3502416 | T/C |
| 1 | 3527882 | C/T |
| 1 | 3538439 | G/A |
| 1 | 3558656 | G/T |
| 1 | 3588709 | C/T |
| 1 | 3600879 | C/T |
| 1 | 3657365 | G/C |
| 1 | 3659711 | C/T |
| 1 | 3660755 | C/T |
| 1 | 3699073 | G/A |
| 1 | 3701226 | C/T |
| 1 | 3742502 | C/T |
| 1 | 3747435 | T/C |
| 1 | 3761645 | T/C |
| 1 | 3765561 | C/T |
| 1 | 3794760 | T/C |
| 1 | 3815959 | G/A |
| 1 | 3868215 | G/A |
| 1 | 3885709 | G/T |
| 1 | 3924386 | C/T |
| 1 | 3929443 | G/A |
| 1 | 3947221 | G/C |
| 1 | 3973478 | G/A |
| 1 | 3982380 | A/G |
| 1 | 3995464 | T/C |
| 1 | 4023936 | C/T |
| 1 | 4025178 | A/G |
| 1 | 4050699 | G/A |
| 1 | 4052724 | T/C |
| 1 | 4080976 | A/T |
| 1 | 4105507 | T/G |
| 1 | 4115190 | C/A |
| 1 | 4118414 | T/C |
| 1 | 4159142 | A/T |
| 1 | 4185334 | A/G |
| 1 | 4186743 | A/G |
| 1 | 4200850 | T/A |
| 1 | 4234007 | A/G |
| 1 | 4268844 | T/G |
| 1 | 4271000 | T/C |
| 1 | 4298184 | G/C |
| 1 | 4315944 | C/T |
| 1 | 4323073 | T/C |
| 1 | 4331128 | A/C |
| 1 | 4360053 | G/A |
| 1 | 4380954 | T/C |
| 1 | 4389983 | T/C |
| 1 | 4431558 | G/A |
| 1 | 4466409 | C/T |
| 1 | 4471540 | C/T |
| 1 | 4473054 | G/A |
| 1 | 4528695 | T/C |
| 1 | 4560885 | T/C |
| 1 | 4595001 | A/G |
| 1 | 4598616 | G/A |
| 1 | 4621291 | G/A |
| 1 | 4629173 | C/T |
| 1 | 4667779 | A/G |
| 1 | 4683029 | G/A |
| 1 | 4719843 | C/G |
| 1 | 4736686 | G/A |
| 1 | 4740786 | T/C |
| 1 | 4749799 | A/T |
| 1 | 4794485 | C/T |
| 1 | 4812456 | G/A |
| 1 | 4827360 | C/T |
| 1 | 4831434 | T/C |
| 1 | 4867558 | A/G |
| 1 | 4881640 | A/C |
| 1 | 4899277 | A/G |
| 1 | 4920136 | T/C |
| 1 | 4962816 | T/G |
| 1 | 4967234 | G/C |
| 1 | 4990386 | C/T |
| 1 | 4992591 | T/C |
| 1 | 5011818 | C/G |
| 1 | 5033717 | C/T |
| 1 | 5054156 | G/C |
| 1 | 5076343 | C/G |
| 1 | 5083795 | A/G |
| 1 | 5112814 | G/A |
| 1 | 5113876 | A/G |
| 1 | 5138187 | C/T |
| 1 | 5164600 | C/T |
| 1 | 5169245 | A/G |
| 1 | 5192375 | C/T |
| 1 | 5203106 | G/A |
| 1 | 5242083 | A/G |
| 1 | 5275072 | C/A |
| 1 | 5307624 | T/G |
| 1 | 5320665 | C/A |
| 1 | 5364208 | C/T |
| 1 | 5399493 | T/A |
| 1 | 5412312 | T/C |
| 1 | 5442363 | C/T |
| 1 | 5475643 | T/C |
| 1 | 5498979 | T/C |
| 1 | 5541817 | G/A |
| 1 | 5563033 | T/C |
| 1 | 5601241 | C/G |
| 1 | 5605908 | T/C |
| 1 | 5633037 | T/C |
| 1 | 5651513 | T/C |
| 1 | 5663439 | A/G |
| 1 | 5699952 | C/A |
| 1 | 5712119 | A/T |
| 1 | 5729184 | G/A |
| 1 | 5751244 | T/C |
| 1 | 5733473 | A/G |
| 1 | 5773334 | C/T |
| 1 | 5788021 | A/G |
| 1 | 5792543 | A/G |
| 1 | 5852674 | C/G |
| 1 | 5855674 | G/A |
| 1 | 5890689 | A/T |
| 1 | 5904572 | A/T |
| 1 | 5932708 | C/T |
| 1 | 5948541 | T/C |
| 1 | 5956622 | T/C |
| 1 | 5974489 | A/G |
| 1 | 6020417 | A/G |
| 1 | 6085467 | A/C |
| 1 | 6095687 | A/G |
| 1 | 6128713 | T/C |
| 1 | 6176712 | G/T |
| 1 | 6234480 | T/A |
| 1 | 6245222 | A/G |
| 1 | 6277421 | T/C |
| 1 | 6300023 | G/A |
| 1 | 6306391 | C/T |
| 1 | 6332224 | A/G |
| 1 | 6381016 | T/C |
| 1 | 6392583 | T/G |
| 1 | 6416827 | G/A |
| 1 | 6439553 | G/A |
| 1 | 6449895 | G/A |
| 1 | 6453395 | G/A |
| 1 | 6476673 | A/C |
| 1 | 6529697 | A/T |
| 1 | 6550693 | C/T |
| 1 | 6571706 | T/A |
| 1 | 6575007 | T/C |
| 1 | 6622915 | T/C |
| 1 | 6624788 | A/C |
| 1 | 6632621 | G/A |
| 1 | 6660831 | T/A |
| 1 | 6666109 | A/G |
| 1 | 6690215 | G/T |
| 1 | 6696073 | A/G |
| 1 | 6738792 | G/A |
| 1 | 6740951 | C/A |
| 1 | 6752754 | A/G |
| 1 | 6803049 | G/A |
| 1 | 6818897 | G/A |
| 1 | 6841471 | A/G |
| 1 | 6847701 | T/C |
| 1 | 6870602 | G/A |
| 1 | 6904483 | T/A |
| 1 | 6921879 | G/T |
| 1 | 6932344 | T/C |
| 1 | 6944221 | C/T |
| 1 | 6962645 | T/A |
| 1 | 6994017 | T/C |
| 1 | 7020965 | C/T |
| 1 | 7059225 | C/T |
| 1 | 7144256 | C/A |
| 1 | 7166241 | C/G |
| 1 | 7171768 | G/A |
| 1 | 7223885 | A/G |
| 1 | 7267383 | G/A |
| 1 | 7292097 | G/A |
| 1 | 7330555 | A/G |
| 1 | 7358639 | C/T |
| 1 | 7385753 | G/A |
| 1 | 7399977 | G/T |
| 1 | 7410631 | A/G |
| 1 | 7418711 | T/C |
| 1 | 7445297 | T/C |
| 1 | 7457048 | T/C |
| 1 | 7472455 | A/C |
| 1 | 7525530 | G/A |
| 1 | 7527277 | T/G |
| 1 | 7556265 | A/G |
| 1 | 7576344 | A/G |
| 1 | 7589256 | A/G |
| 1 | 7616545 | C/A |
| 1 | 7620442 | C/A |
| 1 | 7630722 | T/A |
| 1 | 7672015 | C/T |
| 1 | 7679764 | T/C |
| 1 | 7690368 | A/G |
| 1 | 7701477 | G/A |
| 1 | 7711658 | A/G |
| 1 | 7730763 | G/A |
| 1 | 7767009 | C/A |
| 1 | 7783144 | G/A |
| 1 | 7794316 | A/G |
| 1 | 7867040 | C/T |
| 1 | 7884427 | T/C |
| 1 | 7890529 | G/T |
| 1 | 7937955 | C/T |
| 1 | 7949941 | C/T |
| 1 | 7955748 | C/G |
| 1 | 7983684 | T/G |
| 1 | 7996400 | C/T |
| 1 | 8043002 | G/A |
| 1 | 8067314 | A/G |
| 1 | 8077171 | A/G |
| 1 | 8133760 | T/C |
| 1 | 8145748 | A/T |
| 1 | 8161887 | T/G |
| 1 | 8191098 | G/A |
| 1 | 8237969 | C/A |
| 1 | 8255762 | C/A |
| 1 | 8283955 | G/A |
| 1 | 8296713 | T/C |
| 1 | 8327707 | T/C |
| 1 | 8334129 | T/C |
| 1 | 8368347 | T/C |
| 1 | 8381894 | C/T |
| 1 | 8413423 | A/G |
| 1 | 8421266 | A/C |
| 1 | 8430561 | C/T |
| 1 | 8433825 | G/A |
| 1 | 8466047 | A/G |
| 1 | 8470512 | A/G |
| 1 | 8503869 | C/T |
| 1 | 8545521 | A/G |
| 1 | 8552766 | G/A |
| 1 | 8570476 | C/T |
| 1 | 8615149 | C/T |
| 1 | 8668190 | C/G |
| 1 | 8683425 | G/A |
| 1 | 8687541 | C/T |
| 1 | 8717589 | T/C |
| 1 | 8774769 | C/T |
| 1 | 8818130 | T/G |
| 1 | 8864518 | G/A |
| 1 | 8881423 | C/T |
| 1 | 8966851 | A/C |
| 1 | 8980826 | A/G |
| 1 | 9004116 | C/T |
| 1 | 9061762 | C/T |
| 1 | 9086276 | G/A |
| 1 | 9107773 | A/G |
| 1 | 9144708 | T/C |
| 1 | 9158051 | G/A |
| 1 | 9229114 | A/C |
| 1 | 9231525 | A/G |
| 1 | 9258878 | G/A |
| 1 | 9299451 | C/T |
| 1 | 9315876 | G/A |
| 1 | 9334459 | C/T |
| 1 | 9353719 | T/G |
| 1 | 9370155 | G/A |
| 1 | 9423540 | A/G |
| 1 | 9463212 | C/T |
| 1 | 9515787 | G/A |
| 1 | 9567435 | C/T |
| 1 | 9579401 | G/A |
| 1 | 9623854 | C/T |
| 1 | 9688996 | G/C |
| 1 | 9700951 | A/G |
| 1 | 9711883 | G/C |
| 1 | 9726016 | G/A |
| 1 | 9745692 | C/T |
| 1 | 9763149 | C/T |
| 1 | 9773054 | A/T |
| 1 | 9797175 | A/G |
| 1 | 9808883 | A/G |
| 1 | 9826486 | T/C |
| 1 | 9829683 | A/C |
| 1 | 9866933 | G/T |
| 1 | 9868293 | C/A |
| 1 | 9871203 | G/C |
| 1 | 9906044 | A/G |
| 1 | 9952099 | G/A |
| 1 | 9978215 | G/C |
| 1 | 10003088 | C/A |
| 1 | 10046921 | A/G |
| 1 | 10069419 | T/G |
| 1 | 10071520 | T/C |
| 1 | 10082628 | G/A |
| 1 | 10206217 | G/T |
| 1 | 10220208 | G/A |
| 1 | 10240009 | A/G |
| 1 | 10253336 | A/G |
| 1 | 10283720 | T/C |
| 1 | 10337565 | C/T |
| 1 | 10355827 | C/T |
| 1 | 10407221 | G/A |
| 1 | 10417720 | G/T |
| 1 | 10444456 | A/T |
| 1 | 10449161 | A/C |
| 1 | 10449161 | A/C |
| 1 | 10504307 | C/T |
| 1 | 10556000 | G/A |
| 1 | 10561419 | C/A |
| 1 | 10590980 | C/T |
| 1 | 10621242 | C/T |
| 1 | 10672975 | T/C |
| 1 | 10709613 | C/T |
| 1 | 10711485 | A/G |
| 1 | 10719860 | G/T |
| 1 | 10741056 | G/A |
| 1 | 10798300 | C/A |
| 1 | 10822155 | C/T |
| 1 | 10834756 | T/A |
| 1 | 10848321 | A/G |
| 1 | 10888109 | A/T |
| 1 | 10889982 | G/A |
| 1 | 10896204 | G/A |
| 1 | 10942422 | T/C |
| 1 | 10973718 | A/G |
| 1 | 10977849 | A/G |
| 1 | 10987512 | A/G |
| 1 | 11030635 | C/A |
| 1 | 11047397 | C/T |
| 1 | 11085881 | G/A |
| 1 | 11103130 | C/G |
| 1 | 11147718 | C/A |
| 1 | 11155356 | A/C |
| 1 | 11183750 | A/G |
| 1 | 11198203 | T/C |
| 1 | 11205727 | G/A |
| 1 | 11220997 | C/T |
| 1 | 11225250 | T/C |
| 1 | 11250857 | G/A |
| 1 | 11283115 | C/T |
| 1 | 11339533 | C/A |
| 1 | 11365881 | C/T |
| 1 | 11399632 | A/G |
| 1 | 11401034 | C/T |
| 1 | 11424216 | C/T |
| 1 | 11430090 | G/C |
| 1 | 11431951 | T/C |
| 1 | 11467861 | A/G |
| 1 | 11483765 | G/T |
| 1 | 11505431 | C/A |
| 1 | 11511345 | G/T |
| 1 | 11552518 | C/T |
| 1 | 11583198 | T/A |
| 1 | 11590266 | G/A |
| 1 | 11632342 | T/C |
| 1 | 11657824 | T/A |
| 1 | 11666803 | G/C |
| 1 | 11682952 | C/G |
| 1 | 11695740 | T/C |
| 1 | 11719114 | G/T |
| 1 | 11729672 | C/T |
| 1 | 11735644 | T/A |
| 1 | 11753366 | T/C |
| 1 | 11791186 | G/T |
| 1 | 11869000 | G/A |
| 1 | 11901858 | C/T |
| 1 | 11914306 | C/T |
| 1 | 11940203 | T/A |
| 1 | 11945595 | G/A |
| 1 | 11976710 | T/G |
| 1 | 12004659 | T/G |
| 1 | 12056848 | C/G |
| 1 | 12081712 | T/A |
| 1 | 12100275 | C/T |
| 1 | 12118104 | G/C |
| 1 | 12122300 | C/T |
| 1 | 12153711 | T/C |
| 1 | 12232279 | T/G |
| 1 | 12253437 | G/A |
| 1 | 12276514 | A/G |
| 1 | 12298258 | C/G |
| 1 | 12301664 | C/T |
| 1 | 12315813 | A/G |
| 1 | 12332682 | T/G |
| 1 | 12381205 | G/T |
| 1 | 12395647 | C/A |
| 1 | 12418892 | T/C |
| 1 | 12438764 | G/A |
| 1 | 12448574 | G/A |
| 1 | 12452592 | C/T |
| 1 | 12475109 | G/A |
| 1 | 12483529 | G/A |
| 1 | 12502098 | A/G |
| 1 | 12534335 | C/T |
| 1 | 12539973 | T/C |
| 1 | 12633324 | A/C |
| 1 | 12660225 | T/A |
| 1 | 12666127 | G/T |
| 1 | 12701116 | A/T |
| 1 | 12736153 | G/T |
| 1 | 12762965 | C/T |
| 1 | 12788132 | C/T |
| 1 | 12793313 | T/C |
| 1 | 12834935 | C/T |
| 1 | 12847807 | G/C |
| 1 | 12868659 | G/A |
| 1 | 12893396 | C/G |
| 1 | 12896992 | T/C |
| 1 | 12914582 | G/C |
| 1 | 12956580 | T/C |
| 1 | 12960882 | T/A |
| 1 | 12962651 | C/T |
| 1 | 13002704 | C/A |
| 1 | 13037949 | C/T |
| 1 | 13044286 | G/A |
| 1 | 13052744 | A/G |
| 1 | 13054963 | A/C |
| 1 | 13089109 | C/T |
| 1 | 13119362 | T/C |
| 1 | 13137723 | G/A |
| 1 | 13148055 | T/C |
| 1 | 13185627 | C/G |
| 1 | 13189787 | T/G |
| 1 | 13215332 | G/T |
| 1 | 13215333 | C/T |
| 1 | 13215334 | G/A |
| 1 | 13215335 | C/T |
| 1 | 13215336 | G/A |
| 1 | 13215337 | C/T |
| 1 | 13215338 | G/A |
| 1 | 13272657 | C/T |
| 1 | 13298505 | T/G |
| 1 | 13321640 | C/T |
| 1 | 13355801 | A/T |
| 1 | 13401518 | C/A |
| 1 | 13455561 | G/A |
| 1 | 13478931 | C/T |
| 1 | 13495242 | T/A |
| 1 | 13506909 | G/A |
| 1 | 13538087 | G/A |
| 1 | 13566827 | C/T |
| 1 | 13596639 | C/T |
| 1 | 13600137 | C/A |
| 1 | 13646748 | G/A |
| 1 | 13670957 | G/A |
| 1 | 13684995 | G/A |
| 1 | 13707898 | A/C |
| 1 | 13715721 | A/G |
| 1 | 13741923 | G/A |
| 1 | 13770763 | G/A |
| 1 | 13809140 | C/T |
| 1 | 13819842 | A/T |
| 1 | 14004457 | G/T |
| 1 | 14060495 | G/A |
| 1 | 14075092 | G/A |
| 1 | 14110943 | G/A |
| 1 | 14152572 | G/A |
| 1 | 14162703 | A/G |
| 1 | 14184372 | C/T |
| 1 | 14205904 | G/A |
| 1 | 14214880 | G/A |
| 1 | 14268112 | T/G |
| 1 | 14303041 | C/T |
| 1 | 14312050 | T/C |
| 1 | 14317952 | A/C |
| 1 | 14370296 | G/C |
| 1 | 14433737 | G/A |
| 1 | 14442386 | A/T |
| 1 | 14487087 | G/T |
| 1 | 14500137 | A/G |
| 1 | 14512488 | T/A |
| 1 | 14520560 | T/G |
| 1 | 14528246 | T/G |
| 1 | 14550798 | A/C |
| 1 | 14581809 | G/A |
| 1 | 14614347 | T/C |
| 1 | 14635375 | C/G |
| 1 | 14653394 | T/G |
| 1 | 14687252 | T/C |
| 1 | 14727247 | G/A |
| 1 | 14744255 | A/G |
| 1 | 14778073 | A/G |
| 1 | 14779291 | C/T |
| 1 | 14808063 | G/T |
| 1 | 14854051 | G/A |
| 1 | 14874658 | A/G |
| 1 | 14899796 | A/C |
| 1 | 14938049 | G/A |
| 1 | 14954144 | A/G |
| 1 | 14981305 | G/A |
| 1 | 14983906 | A/C |
| 1 | 15013507 | G/A |
| 1 | 15015581 | G/A |
| 1 | 15036507 | T/A |
| 1 | 15056869 | G/A |
| 1 | 15078138 | T/C |
| 1 | 15092715 | T/C |
| 1 | 15129718 | C/T |
| 1 | 15171016 | C/T |
| 1 | 15185614 | C/A |
| 1 | 15210971 | C/A |
| 1 | 15239573 | C/T |
| 1 | 15244085 | G/A |
| 1 | 15279279 | C/T |
| 1 | 15282655 | G/A |
| 1 | 15303050 | G/A |
| 1 | 15313366 | T/C |
| 1 | 15343043 | G/A |
| 1 | 15345189 | A/G |
| 1 | 15369644 | C/T |
| 1 | 15380052 | G/A |
| 1 | 15400795 | G/A |
| 1 | 15406167 | C/G |
| 1 | 15426530 | G/A |
| 1 | 15437011 | G/T |
| 1 | 15453391 | A/G |
| 1 | 15470394 | G/A |
| 1 | 15495865 | C/G |
| 1 | 15501962 | C/G |
| 1 | 15523121 | C/T |
| 1 | 15564729 | A/G |
| 1 | 15572965 | A/C |
| 1 | 15584977 | C/G |
| 1 | 15611843 | A/G |
| 1 | 15613948 | G/A |
| 1 | 15638070 | G/A |
| 1 | 15650585 | T/C |
| 1 | 15699184 | G/T |
| 1 | 15701399 | T/C |
| 1 | 15724448 | G/A |
| 1 | 15726636 | A/G |
| 1 | 15801268 | C/A |
| 1 | 15824850 | C/T |
| 1 | 15845767 | G/A |
| 1 | 15848828 | C/T |
| 1 | 15895057 | T/A |
| 1 | 15913378 | C/T |
| 1 | 15929434 | G/A |
| 1 | 15957868 | T/A |
| 1 | 15961215 | G/A |
| 1 | 15964166 | A/T |
| 1 | 15997016 | T/A |
| 1 | 16022230 | C/A |
| 1 | 16058508 | C/T |
| 1 | 16072691 | C/G |
| 1 | 16096654 | G/C |
| 1 | 16100368 | T/A |
| 1 | 16122629 | C/T |
| 1 | 16124485 | A/C |
| 1 | 16142144 | C/A |
| 1 | 16192634 | G/T |
| 1 | 16218980 | T/A |
| 1 | 16225544 | A/T |
| 1 | 16233713 | C/T |
| 1 | 16262578 | C/G |
| 1 | 16294518 | G/A |
| 1 | 16302643 | G/A |
| 1 | 16320249 | T/C |
| 1 | 16346585 | C/G |
| 1 | 16352025 | G/T |
| 1 | 16356452 | G/A |
| 1 | 16387568 | C/G |
| 1 | 16390715 | A/C |
| 1 | 16436625 | A/T |
| 1 | 16444432 | C/A |
| 1 | 16463640 | C/T |
| 1 | 16489822 | G/A |
| 1 | 16508356 | G/A |
| 1 | 16514981 | G/A |
| 1 | 16557521 | G/C |
| 1 | 16566625 | C/T |
| 1 | 16612824 | G/A |
| 1 | 16619435 | C/G |
| 1 | 16649008 | C/A |
| 1 | 16661491 | T/A |
| 1 | 16667068 | C/T |
| 1 | 16691020 | G/A |
| 1 | 16695911 | G/A |
| 1 | 16717483 | A/T |
| 1 | 16725286 | C/A |
| 1 | 16741896 | G/A |
| 1 | 16748378 | C/T |
| 1 | 16780609 | C/G |
| 1 | 16812917 | C/T |
| 1 | 16833295 | G/A |
| 1 | 16835295 | C/G |
| 1 | 16868891 | G/A |
| 1 | 16883721 | T/C |
| 1 | 16883722 | A/G |
| 1 | 16883723 | A/G |
| 1 | 16883724 | A/G |
| 1 | 16883725 | A/G |
| 1 | 16883726 | A/G |
| 1 | 16883727 | A/G |
| 1 | 16883728 | A/T |
| 1 | 16883729 | A/T |
| 1 | 16883730 | A/T |
| 1 | 16963217 | A/C |
| 1 | 16971159 | C/T |
| 1 | 16984581 | G/C |
| 1 | 17008052 | A/G |
| 1 | 17016698 | T/C |
| 1 | 17045821 | T/G |
| 1 | 17069018 | G/A |
| 1 | 17072293 | T/A |
| 1 | 17129547 | C/T |
| 1 | 17134591 | G/A |
| 1 | 17175035 | C/T |
| 1 | 17195201 | T/C |
| 1 | 17254554 | A/G |
| 1 | 17257336 | G/A |
| 1 | 17284181 | C/T |
| 1 | 17306185 | C/T |
| 1 | 17321273 | C/T |
| 1 | 17354139 | G/A |
| 1 | 17368592 | T/C |
| 1 | 17370878 | G/A |
| 1 | 17385934 | T/G |
| 1 | 17414051 | G/A |
| 1 | 17472614 | G/A |
| 1 | 17495486 | G/A |
| 1 | 17514657 | C/T |
| 1 | 17530161 | T/C |
| 1 | 17594035 | A/C |
| 1 | 17635044 | T/C |
| 1 | 17643956 | C/G |
| 1 | 17676419 | C/T |
| 1 | 17760408 | G/A |
| 1 | 17771926 | C/T |
| 1 | 17790633 | C/T |
| 1 | 17834844 | C/T |
| 1 | 17856063 | C/T |
| 1 | 17869794 | C/T |
| 1 | 17964318 | C/T |
| 1 | 18023431 | A/C |
| 1 | 18100715 | G/A |
| 1 | 18120068 | T/C |
| 1 | 18162396 | T/C |
| 1 | 18182934 | C/G |
| 1 | 18221311 | C/G |
| 1 | 18257715 | G/A |
| 1 | 18290993 | G/A |
| 1 | 18310469 | G/A |
| 1 | 18348612 | T/A |
| 1 | 18385138 | A/G |
| 1 | 18479962 | A/C |
| 1 | 18482553 | A/G |
| 1 | 18529346 | A/T |
| 1 | 18529347 | G/A |
| 1 | 18529348 | G/A |
| 1 | 18529349 | G/A |
| 1 | 18609416 | T/C |
| 1 | 18621318 | C/T |
| 1 | 18643387 | A/G |
| 1 | 18661262 | C/T |
| 1 | 18688989 | T/A |
| 1 | 18697243 | A/G |
| 1 | 18703480 | C/A |
| 1 | 18722983 | G/A |
| 1 | 18727059 | A/C |
| 1 | 18776728 | G/A |
| 1 | 18817425 | A/T |
| 1 | 18824969 | G/T |
| 1 | 18851660 | G/T |
| 1 | 18864642 | A/T |
| 1 | 18871727 | T/G |
| 1 | 18896071 | G/A |
| 1 | 18900390 | C/A |
| 1 | 18922851 | G/C |
| 1 | 18947206 | A/G |
| 1 | 18958048 | G/A |
| 1 | 19002132 | G/A |
| 1 | 19004822 | A/T |
| 1 | 19030488 | C/T |
| 1 | 19051180 | C/T |
| 1 | 19072015 | A/G |
| 1 | 19090698 | T/C |
| 1 | 19096306 | C/T |
| 1 | 19111736 | G/A |
| 1 | 19119581 | C/T |
| 1 | 19158704 | C/T |
| 1 | 19173910 | T/C |
| 1 | 19193216 | A/T |
| 1 | 19219825 | G/A |
| 1 | 19246203 | A/G |
| 1 | 19259472 | G/A |
| 1 | 19261350 | C/T |
| 1 | 19269922 | A/G |
| 1 | 19313423 | G/A |
| 1 | 19315690 | C/T |
| 1 | 19327168 | A/T |
| 1 | 19333414 | C/T |
| 1 | 19359554 | G/A |
| 1 | 19395201 | G/A |
| 1 | 19401799 | A/T |
| 1 | 19425438 | A/G |
| 1 | 19428345 | G/A |
| 1 | 19450331 | A/C |
| 1 | 19464431 | A/G |
| 1 | 19470767 | T/C |
| 1 | 19485115 | A/G |
| 1 | 19500754 | G/C |
| 1 | 19522010 | A/G |
| 1 | 19545155 | G/A |
| 1 | 19549122 | G/A |
| 1 | 19585521 | C/T |
| 1 | 19601602 | G/A |
| 1 | 19616584 | C/T |
| 1 | 19638765 | G/T |
| 1 | 19660660 | G/A |
| 1 | 19680275 | A/G |
| 1 | 19716737 | C/T |
| 1 | 19724013 | G/A |
| 1 | 19763060 | G/A |
| 1 | 19768991 | A/T |
| 1 | 19771926 | C/T |
| 1 | 19790241 | A/G |
| 1 | 19818988 | G/A |
| 1 | 19829478 | C/T |
| 1 | 19841081 | A/C |
| 1 | 19901203 | G/A |
| 1 | 19975046 | C/T |
| 1 | 20000653 | T/C |
| 1 | 20023176 | A/T |
| 1 | 20043086 | C/G |
| 1 | 20064008 | T/A |
| 1 | 20084541 | T/C |
| 1 | 20090642 | C/T |
| 1 | 20101921 | C/T |
| 1 | 20124762 | G/T |
| 1 | 20140122 | A/C |
| 1 | 20182748 | C/T |
| 1 | 20184647 | C/T |
| 1 | 20218640 | G/A |
| 1 | 20220593 | T/A |
| 1 | 20261845 | C/G |
| 1 | 20287516 | G/A |
| 1 | 20297301 | C/T |
| 1 | 20310809 | T/C |
| 1 | 20312154 | G/T |
| 1 | 20354157 | C/G |
| 1 | 20355828 | G/A |
| 1 | 20355829 | C/T |
| 1 | 20355830 | C/T |
| 1 | 20355831 | C/T |
| 1 | 20419887 | A/G |
| 1 | 20430138 | C/T |
| 1 | 20462829 | G/A |
| 1 | 20465317 | G/T |
| 1 | 20502824 | G/A |
| 1 | 20505873 | A/G |
| 1 | 20532447 | C/G |
| 1 | 20533484 | T/C |
| 1 | 20577655 | T/G |
| 1 | 20584245 | A/C |
| 1 | 20607585 | A/C |
| 1 | 20610691 | G/A |
| 1 | 20632393 | T/C |
| 1 | 20646037 | A/G |
| 1 | 20682516 | G/C |
| 1 | 20700369 | C/G |
| 1 | 20713985 | C/T |
| 1 | 20751437 | A/G |
| 1 | 20780458 | A/G |
| 1 | 20791412 | A/C |
| 1 | 20816508 | C/A |
| 1 | 20828010 | G/C |
| 1 | 20841704 | C/T |
| 1 | 20853610 | C/A |
| 1 | 20884486 | T/G |
| 1 | 20899661 | C/A |
| 1 | 20929502 | C/G |
| 1 | 20936463 | A/C |
| 1 | 20966922 | G/T |
| 1 | 20983587 | G/A |
| 1 | 20997319 | C/G |
| 1 | 21009211 | A/T |
| 1 | 21028071 | A/G |
| 1 | 21030911 | G/T |
| 1 | 21047798 | A/G |
| 1 | 21089811 | A/T |
| 1 | 21091035 | C/T |
| 1 | 21094981 | T/C |
| 1 | 21120864 | G/C |
| 1 | 21125178 | T/C |
| 1 | 21156911 | T/A |
| 1 | 21190173 | G/A |
| 1 | 21206510 | G/C |
| 1 | 21226925 | G/A |
| 1 | 21257735 | C/T |
| 1 | 21271824 | C/T |
| 1 | 21282376 | T/G |
| 1 | 21304497 | G/A |
| 1 | 21370979 | G/A |
| 1 | 21373558 | G/A |
| 1 | 21408550 | G/A |
| 1 | 21410832 | G/A |
| 1 | 21426712 | G/C |
| 1 | 21452365 | C/T |
| 1 | 21462981 | A/G |
| 1 | 21497951 | G/C |
| 1 | 21503427 | G/C |
| 1 | 21512528 | C/T |
| 1 | 21517207 | C/G |
| 1 | 21543304 | C/T |
| 1 | 21569517 | A/T |
| 1 | 21596833 | G/A |
| 1 | 21605384 | C/T |
| 1 | 21607525 | C/T |
| 1 | 21631432 | T/A |
| 1 | 21655063 | G/A |
| 1 | 21677146 | G/A |
| 1 | 21702602 | G/A |
| 1 | 21719241 | A/G |
| 1 | 21727343 | T/C |
| 1 | 21753996 | C/T |
| 1 | 21781537 | G/A |
| 1 | 21787424 | G/A |
| 1 | 21817811 | T/G |
| 1 | 21915458 | G/A |
| 1 | 21922051 | C/T |
| 1 | 21961463 | G/C |
| 1 | 22000213 | T/C |
| 1 | 22031228 | C/T |
| 1 | 22036350 | T/C |
| 1 | 22068816 | T/C |
| 1 | 22147503 | A/G |
| 1 | 22173586 | G/A |
| 1 | 22196898 | T/C |
| 1 | 22224544 | C/T |
| 1 | 22335235 | A/G |
| 1 | 22377942 | C/T |
| 1 | 22396103 | C/T |
| 1 | 22432303 | T/A |
| 1 | 22436919 | C/T |
| 1 | 22469878 | A/G |
| 1 | 22491190 | T/G |
| 1 | 22523190 | C/T |
| 1 | 22524377 | C/T |
| 1 | 22555874 | C/T |
| 1 | 22570359 | T/C |
| 1 | 22615331 | G/A |
| 1 | 22618016 | A/T |
| 1 | 22628140 | G/A |
| 1 | 22630171 | G/A |
| 1 | 22656062 | A/G |
| 1 | 22682317 | G/A |
| 1 | 22688471 | C/A |
| 1 | 22712290 | G/T |
| 1 | 22719489 | A/G |
| 1 | 22754870 | G/A |
| 1 | 22766571 | C/T |
| 1 | 22780352 | G/A |
| 1 | 22795673 | C/T |
| 1 | 22805756 | C/T |
| 1 | 22845583 | C/T |
| 1 | 22849620 | G/T |
| 1 | 22869915 | G/A |
| 1 | 22872055 | C/T |
| 1 | 22921294 | C/T |
| 1 | 22953797 | G/T |
| 1 | 22966517 | G/T |
| 1 | 22986840 | G/T |
| 1 | 23025942 | T/C |
| 1 | 23042732 | G/A |
| 1 | 23059617 | T/C |
| 1 | 23091594 | C/G |
| 1 | 23142846 | C/G |
| 1 | 23145055 | G/T |
| 1 | 23179742 | A/C |
| 1 | 23181642 | A/C |
| 1 | 23211640 | C/T |
| 1 | 23219462 | A/C |
| 1 | 23237741 | C/T |
| 1 | 23255036 | A/T |
| 1 | 23281760 | G/A |
| 1 | 23310778 | C/T |
| 1 | 23313387 | G/A |
| 1 | 23345191 | T/C |
| 1 | 23345192 | A/T |
| 1 | 23345193 | A/T |
| 1 | 23345194 | A/T |
| 1 | 23429755 | G/T |
| 1 | 23434033 | C/T |
| 1 | 23439758 | T/C |
| 1 | 23467614 | G/A |
| 1 | 23473202 | C/T |
| 1 | 23499006 | G/A |
| 1 | 23502306 | G/A |
| 1 | 23536264 | T/A |
| 1 | 23554744 | C/T |
| 1 | 23577945 | A/G |
| 1 | 23588476 | C/T |
| 1 | 23593130 | A/C |
| 1 | 23615848 | T/C |
| 1 | 23665911 | G/C |
| 1 | 23674478 | C/A |
| 1 | 23675940 | G/A |
| 1 | 23711527 | C/T |
| 1 | 23721008 | A/G |
| 1 | 23737155 | A/C |
| 1 | 23742626 | A/G |
| 1 | 23767515 | A/G |
| 1 | 23788599 | C/A |
| 1 | 23797081 | C/A |
| 1 | 23832364 | T/C |
| 1 | 23895499 | G/A |
| 1 | 23900981 | T/C |
| 1 | 23910834 | A/G |
| 1 | 23935614 | G/A |
| 1 | 23940570 | G/A |
| 1 | 23949284 | T/C |
| 1 | 23970464 | C/T |
| 1 | 24016192 | G/A |
| 1 | 24048824 | C/T |
| 1 | 24067734 | A/G |
| 1 | 24102530 | C/T |
| 1 | 24136285 | A/G |
| 1 | 24176105 | T/C |
| 1 | 24179660 | C/T |
| 1 | 24195493 | C/T |
| 1 | 24202723 | G/A |
| 1 | 24220677 | C/T |
| 1 | 24255356 | G/A |
| 1 | 24288238 | C/T |
| 1 | 24302947 | A/C |
| 1 | 24317568 | G/A |
| 1 | 24357324 | T/G |
| 1 | 24377871 | C/T |
| 1 | 24403773 | G/A |
| 1 | 24429019 | C/A |
| 1 | 24444984 | A/G |
| 1 | 24451287 | G/C |
| 1 | 24464622 | C/T |
| 1 | 24507176 | C/T |
| 1 | 24510043 | C/T |
| 1 | 24540194 | C/T |
| 1 | 24559432 | A/G |
| 1 | 24577333 | C/T |
| 1 | 24625734 | G/A |
| 1 | 24641948 | A/G |
| 1 | 24645579 | T/G |
| 1 | 24665546 | T/C |
| 1 | 24681500 | C/T |
| 1 | 24702035 | A/C |
| 1 | 24708830 | C/G |
| 1 | 24728111 | C/T |
| 1 | 24735185 | A/T |
| 1 | 24768583 | T/C |
| 1 | 24771643 | C/G |
| 1 | 24781118 | T/A |
| 1 | 24808148 | C/T |
| 1 | 24808149 | T/C |
| 1 | 24808150 | T/C |
| 1 | 24808151 | T/C |
| 1 | 24861334 | C/T |
| 1 | 24873377 | G/T |
| 1 | 24898168 | G/A |
| 1 | 24921185 | G/A |
| 1 | 24940701 | C/T |
| 1 | 24955141 | G/A |
| 1 | 24977096 | A/G |
| 1 | 25022194 | A/G |
| 1 | 25033538 | T/A |
| 1 | 25067866 | C/T |
| 1 | 25126667 | C/T |
| 1 | 25139386 | G/C |
| 1 | 25154999 | A/G |
| 1 | 25168376 | A/G |
| 1 | 25180091 | G/T |
| 1 | 25226418 | G/T |
| 1 | 25251205 | C/T |
| 1 | 25280665 | C/G |
| 1 | 25289679 | G/A |
| 1 | 25292887 | T/C |
| 1 | 25308925 | T/C |
| 1 | 25360384 | G/A |
| 1 | 25364356 | G/A |
| 1 | 25381067 | C/T |
| 1 | 25405973 | C/T |
| 1 | 25435737 | C/T |
| 1 | 25455229 | C/T |
| 1 | 25460540 | C/T |
| 1 | 25480943 | T/G |
| 1 | 25483596 | T/C |
| 1 | 25520243 | G/A |
| 1 | 25552111 | C/T |
| 1 | 25553832 | C/T |
| 1 | 25576037 | C/T |
| 1 | 25583373 | C/G |
| 1 | 25597445 | A/G |
| 1 | 25599244 | C/T |
| 1 | 25620278 | C/T |
| 1 | 25649363 | C/T |
| 1 | 25649364 | G/A |
| 1 | 25649365 | G/A |
| 1 | 25649366 | G/A |
| 1 | 25699143 | A/G |
| 1 | 25702587 | T/C |
| 1 | 25719065 | G/T |
| 1 | 25731754 | G/A |
| 1 | 25750125 | T/C |
| 1 | 25767335 | C/T |
| 1 | 25771359 | G/A |
| 1 | 25820003 | A/G |
| 1 | 25828278 | A/G |
| 1 | 25858935 | C/A |
| 1 | 25863534 | G/T |
| 1 | 25863535 | C/T |
| 1 | 25863536 | C/T |
| 1 | 25863537 | C/T |
| 1 | 25926907 | A/T |
| 1 | 25956976 | A/T |
| 1 | 26000329 | A/G |
| 1 | 26015128 | C/T |
| 1 | 26020591 | A/G |
| 1 | 26058947 | T/C |
| 1 | 26067274 | A/C |
| 1 | 26081548 | A/G |
| 1 | 26090233 | A/G |
| 1 | 26101535 | T/C |
| 1 | 26125789 | C/A |
| 1 | 26139052 | C/T |
| 1 | 26178236 | T/C |
| 1 | 26195708 | A/T |
| 1 | 26240655 | C/T |
| 1 | 26242298 | A/C |
| 1 | 26269502 | G/A |
| 1 | 26282158 | C/T |
| 1 | 26286594 | T/C |
| 1 | 26318048 | T/C |
| 1 | 26334410 | T/C |
| 1 | 26344160 | C/T |
| 1 | 26391313 | T/C |
| 1 | 26426684 | C/A |
| 1 | 26439214 | A/G |
| 1 | 26467156 | T/C |
| 1 | 26470840 | C/T |
| 1 | 26510677 | A/G |
| 1 | 26520828 | A/G |
| 1 | 26531849 | T/G |
| 1 | 26555282 | C/T |
| 1 | 26567226 | G/A |
| 1 | 26626415 | G/A |
| 1 | 26682638 | T/C |
| 1 | 26692160 | A/T |
| 1 | 26715623 | C/T |
| 1 | 26717651 | C/A |
| 1 | 26742173 | G/A |
| 1 | 26762559 | C/T |
| 1 | 26762560 | A/G |
| 1 | 26763313 | C/T |
| 1 | 26847181 | C/T |
| 1 | 26877169 | A/T |
| 1 | 26890612 | A/G |
| 1 | 26890613 | A/G |
| 1 | 26890614 | A/G |
| 1 | 26890615 | A/G |
| 1 | 27003738 | C/T |
| 1 | 27003739 | T/G |
| 1 | 27003740 | T/G |
| 1 | 27003741 | T/G |
| 1 | 27063414 | G/A |
| 1 | 27069543 | C/T |
| 1 | 27093053 | A/T |
| 1 | 27122768 | G/A |
| 1 | 27132879 | G/A |
| 1 | 27177100 | G/A |
| 1 | 27259912 | A/C |
| 1 | 27267107 | C/T |
| 1 | 27291906 | G/A |
| 1 | 27309628 | G/T |
| 1 | 27329022 | G/A |
| 1 | 37345943 | C/T |
| 1 | 27358133 | G/A |
| 1 | 27361297 | C/T |
| 1 | 27389812 | C/G |
| 1 | 27395790 | C/G |
| 1 | 27440987 | C/T |
| 1 | 27443634 | G/T |
| 1 | 27461317 | C/T |
| 1 | 27469122 | G/A |
| 1 | 27526098 | C/T |
| 1 | 27528383 | C/T |
| 1 | 27549595 | G/T |
| 1 | 27568854 | C/T |
| 1 | 27572427 | C/T |
| 1 | 27599255 | C/T |
| 1 | 27616459 | A/T |
| 1 | 27646742 | G/C |
| 1 | 27663367 | T/C |
| 1 | 27678963 | C/T |
| 1 | 27691651 | C/T |
| 1 | 27699124 | C/T |
| 1 | 27739647 | G/A |
| 1 | 27744422 | T/A |
| 1 | 27802739 | A/G |
| 1 | 27809855 | G/C |
| 1 | 27829446 | G/A |
| 1 | 27893696 | G/A |
| 1 | 27924682 | C/T |
| 1 | 27933964 | T/A |
| 1 | 27947421 | C/T |
| 1 | 27968127 | G/A |
| 1 | 27999474 | G/A |
| 1 | 28029928 | G/A |
| 1 | 28038694 | T/C |
| 1 | 28077397 | G/C |
| 1 | 28118404 | T/C |
| 1 | 28156700 | C/T |
| 1 | 28158729 | A/G |
| 1 | 28173434 | G/A |
| 1 | 28200604 | T/C |
| 1 | 28240415 | A/C |
| 1 | 28256218 | T/C |
| 1 | 28281211 | A/T |
| 1 | 28310672 | G/T |
| 1 | 28320237 | C/A |
| 1 | 28366850 | G/C |
| 1 | 28369164 | A/T |
| 1 | 28388803 | C/T |
| 1 | 28428005 | G/C |
| 1 | 28438764 | G/A |
| 1 | 28452776 | T/C |
| 1 | 28474072 | G/A |
| 1 | 28486033 | A/T |
| 1 | 28518734 | T/G |
| 1 | 28556945 | G/A |
| 1 | 28561296 | G/A |
| 1 | 28562778 | A/C |
| 1 | 28619047 | C/T |
| 1 | 28632906 | T/C |
| 1 | 28657143 | T/C |
| 1 | 28693916 | G/C |
| 1 | 28695071 | T/C |
| 1 | 28732850 | G/A |
| 1 | 28744668 | G/A |
| 1 | 28792647 | A/G |
| 1 | 28804208 | A/G |
| 1 | 28806019 | A/G |
| 1 | 28838001 | A/G |
| 1 | 28861093 | C/A |
| 1 | 28862872 | A/G |
| 1 | 28890929 | C/G |
| 1 | 28918735 | T/C |
| 1 | 28920666 | A/T |
| 1 | 28924196 | T/C |
| 1 | 28962472 | A/G |
| 1 | 29007047 | A/C |
| 1 | 29012731 | C/T |
| 1 | 29036942 | G/C |
| 1 | 29045981 | G/C |
| 1 | 29053700 | G/A |
| 1 | 29099990 | G/A |
| 1 | 29111422 | C/T |
| 1 | 29151916 | T/C |
| 1 | 29154187 | A/C |
| 1 | 29180968 | C/T |
| 1 | 29182249 | A/T |
| 1 | 29192538 | G/A |
| 1 | 29232950 | T/C |
| 1 | 29264918 | T/C |
| 1 | 29282022 | A/T |
| 1 | 29293510 | G/A |
| 1 | 29317935 | A/G |
| 1 | 29334020 | T/C |
| 1 | 29340088 | C/G |
| 1 | 29358952 | C/T |
| 1 | 29377797 | A/G |
| 1 | 29383501 | A/G |
| 1 | 29406014 | G/A |
| 1 | 29414194 | A/G |
| 1 | 29441661 | A/G |
| 1 | 29444942 | A/G |
| 1 | 29484548 | T/C |
| 1 | 29486347 | T/C |
| 1 | 29506312 | T/G |
| 1 | 29513468 | T/A |
| 1 | 29524312 | G/A |
| 1 | 29535897 | C/T |
| 1 | 29558580 | G/A |
| 1 | 29604080 | T/A |
| 1 | 29607638 | C/T |
| 1 | 29618846 | G/A |
| 1 | 29647626 | C/T |
| 1 | 29652356 | C/T |
| 1 | 29681853 | C/T |
| 1 | 29698148 | C/T |
| 1 | 29708216 | C/T |
| 1 | 29740889 | C/T |
| 1 | 29744131 | C/T |
| 1 | 29768921 | C/T |
| 1 | 29807783 | G/A |
| 1 | 29817084 | C/T |
| 1 | 29826931 | C/T |
| 1 | 29846906 | C/T |
| 1 | 29853996 | C/T |
| 1 | 29889347 | C/A |
| 1 | 29933355 | C/T |
| 1 | 29955977 | C/T |
| 1 | 29960305 | C/T |
| 1 | 29971738 | C/G |
| 1 | 29975155 | T/C |
| 1 | 30009841 | G/T |
| 1 | 30050109 | A/C |
| 1 | 30072786 | A/G |
| 1 | 30093030 | C/T |
| 1 | 30122262 | C/A |
| 1 | 30133070 | C/T |
| 1 | 30172869 | A/C |
| 1 | 30206751 | G/A |
| 1 | 30220650 | C/G |
| 1 | 30241506 | C/G |
| 1 | 30274308 | C/G |
| 1 | 30275447 | A/G |
| 1 | 30300360 | T/C |
| 1 | 30307429 | T/C |
| 1 | 30356789 | G/T |
| 1 | 30361464 | T/C |
| 1 | 30392703 | A/G |
| 1 | 30415935 | A/G |
| 1 | 30448488 | T/A |
| 1 | 30455125 | C/T |
| 1 | 30475215 | A/G |
| 1 | 30494839 | A/G |
| 1 | 30495848 | T/C |
| 1 | 30511931 | A/G |
| 1 | 30544963 | C/T |
| 1 | 30551288 | T/C |
| 1 | 30598399 | A/T |
| 1 | 30642718 | G/A |
| 1 | 30717448 | A/G |
| 1 | 30723922 | C/G |
| 1 | 30778805 | G/A |
| 1 | 30782918 | A/G |
| 1 | 30793922 | A/G |
| 1 | 30813061 | T/C |
| 1 | 30837039 | A/G |
| 1 | 30842270 | C/T |
| 1 | 30843334 | G/A |
| 1 | 30883601 | G/C |
| 1 | 30896211 | A/G |
| 1 | 30915344 | C/A |
| 1 | 30939239 | A/G |
| 1 | 30957819 | A/C |
| 1 | 30963520 | A/G |
| 1 | 30976573 | T/G |
| 1 | 31001743 | G/A |
| 1 | 31024303 | G/A |
| 1 | 31061007 | C/T |
| 1 | 31074328 | T/G |
| 1 | 31093066 | A/G |
| 1 | 31101045 | T/C |
| 1 | 31121625 | G/C |
| 1 | 31132560 | G/A |
| 1 | 31140616 | C/T |
| 1 | 31157983 | T/C |
| 1 | 31213907 | A/T |
| 1 | 31222628 | C/T |
| 1 | 31259999 | A/G |
| 1 | 31261200 | C/G |
| 1 | 31264403 | G/A |
| 1 | 31294933 | C/G |
| 1 | 31336837 | T/C |
| 1 | 31345469 | A/G |
| 1 | 31370747 | G/A |
| 1 | 31408365 | G/C |
| 1 | 31457430 | C/T |
| 1 | 31469513 | A/G |
| 1 | 31481195 | G/A |
| 1 | 31484936 | A/T |
| 1 | 31516756 | C/T |
| 1 | 31528641 | G/T |
| 1 | 31538266 | C/T |
| 1 | 31542200 | G/C |
| 1 | 31570334 | G/C |
| 1 | 31615660 | T/A |
| 1 | 31639044 | C/T |
| 1 | 31672869 | C/T |
| 1 | 31685684 | C/G |
| 1 | 31706517 | T/G |
| 1 | 31710581 | C/A |
| 1 | 31747767 | G/A |
| 1 | 31771103 | C/A |
| 1 | 31805198 | C/T |
| 1 | 31814072 | A/G |
| 1 | 31834915 | T/A |
| 1 | 31839796 | G/A |
| 1 | 31896247 | C/T |
| 1 | 31963297 | C/T |
| 1 | 32002636 | G/A |
| 1 | 32032459 | T/G |
| 1 | 32050453 | G/A |
| 1 | 32078943 | C/T |
| 1 | 32082275 | G/A |
| 1 | 32109810 | A/T |
| 1 | 32129210 | G/A |
| 1 | 32144891 | A/G |
| 1 | 32188274 | C/A |
| 1 | 32196267 | C/A |
| 1 | 32203104 | T/C |
| 1 | 32222381 | C/A |
| 1 | 32251352 | C/T |
| 1 | 32289155 | C/T |
| 1 | 32337822 | T/G |
| 1 | 32366501 | C/G |
| 1 | 32373694 | G/A |
| 1 | 32384265 | T/C |
| 1 | 32405377 | T/G |
| 1 | 32454190 | G/C |
| 1 | 32457471 | T/C |
| 1 | 32467811 | A/G |
| 1 | 32508108 | G/A |
| 1 | 32531998 | T/G |
| 1 | 32563283 | T/C |
| 1 | 32578313 | G/A |
| 1 | 32592360 | C/T |
| 1 | 32624760 | G/A |
| 1 | 32649776 | A/C |
| 1 | 32662081 | T/C |
| 1 | 32679656 | C/T |
| 1 | 32725272 | C/T |
| 1 | 32726958 | G/A |
| 1 | 32732314 | A/C |
| 1 | 32762168 | T/C |
| 1 | 32783776 | A/G |
| 1 | 32806965 | A/G |
| 1 | 32814775 | C/T |
| 1 | 32821502 | A/G |
| 1 | 32838481 | G/A |
| 1 | 32868141 | G/C |
| 1 | 32880603 | T/C |
| 1 | 32904597 | G/A |
| 1 | 32937338 | A/G |
| 1 | 32943401 | A/G |
| 1 | 32950574 | T/C |
| 1 | 32972041 | G/A |
| 1 | 32997872 | G/A |
| 1 | 33000498 | T/A |
| 1 | 33012358 | G/T |
| 1 | 33039647 | G/A |
| 1 | 33056143 | G/T |
| 1 | 33062876 | A/G |
| 1 | 33065939 | T/C |
| 1 | 33132271 | G/A |
| 1 | 33147629 | T/A |
| 1 | 33155237 | G/A |
| 1 | 33167900 | G/C |
| 1 | 33181170 | G/A |
| 1 | 33216619 | C/T |
| 1 | 33224726 | A/G |
| 1 | 33244855 | T/C |
| 1 | 33255052 | G/A |
| 1 | 33274073 | T/C |
| 1 | 33278493 | T/C |
| 1 | 33307846 | T/C |
| 1 | 33351537 | T/C |
| 1 | 33366851 | A/G |
| 1 | 33410471 | G/A |
| 1 | 33414331 | C/A |
| 1 | 33420255 | G/A |
| 1 | 33423118 | T/C |
| 1 | 33453389 | G/A |
| 1 | 33494826 | C/T |
| 1 | 33501015 | T/C |
| 1 | 33528092 | C/A |
| 1 | 33536161 | C/T |
| 1 | 33541962 | G/A |
| 1 | 33567630 | C/T |
| 1 | 33576264 | T/A |
| 1 | 33591519 | G/A |
| 1 | 33600783 | T/C |
| 1 | 33607504 | A/G |
| 1 | 33634163 | A/G |
| 1 | 33636214 | C/T |
| 1 | 33674905 | C/T |
| 1 | 33683253 | G/A |
| 1 | 33707653 | C/T |
| 1 | 33711329 | C/A |
| 1 | 33762269 | G/T |
| 1 | 33767197 | A/C |
| 1 | 33787121 | C/T |
| 1 | 33826339 | T/C |
| 1 | 33831757 | T/C |
| 1 | 33853024 | G/A |
| 1 | 33868819 | C/T |
| 1 | 33889077 | C/T |
| 1 | 33895066 | G/A |
| 1 | 33907422 | G/A |
| 1 | 33956269 | G/T |
| 1 | 33964250 | G/A |
| 1 | 33986651 | C/T |
| 1 | 33992760 | T/C |
| 1 | 34004730 | A/G |
| 1 | 34029052 | C/T |
| 1 | 34043506 | A/G |
| 1 | 34078257 | T/G |
| 1 | 34082019 | T/G |
| 1 | 34083606 | A/T |
| 1 | 34125183 | G/A |
| 1 | 34136802 | C/T |
| 1 | 34144311 | C/A |
| 1 | 34175585 | G/A |
| 1 | 34178638 | G/A |
| 1 | 34220729 | T/C |
| 1 | 34250219 | A/T |
| 1 | 34276114 | C/T |
| 1 | 34299572 | C/T |
| 1 | 34308184 | T/C |
| 1 | 34339640 | C/T |
| 1 | 34355751 | C/T |
| 1 | 34385128 | A/G |
| 1 | 34421031 | C/T |
| 1 | 34448582 | G/A |
| 1 | 34450499 | A/G |
| 1 | 34470970 | C/A |
| 1 | 34573118 | C/T |
| 1 | 34619960 | C/T |
| 1 | 34646796 | C/T |
| 1 | 34669128 | C/T |
| 1 | 34688125 | C/T |
| 1 | 34719395 | T/C |
| 1 | 34755202 | G/A |
| 1 | 34791795 | C/A |
| 1 | 34831543 | A/G |
| 1 | 34865875 | A/G |
| 1 | 34899735 | A/T |
| 1 | 34930485 | C/T |
| 1 | 34945821 | C/T |
| 1 | 35005858 | A/T |
| 1 | 35027153 | C/T |
| 1 | 35030759 | T/C |
| 1 | 35051261 | T/C |
| 1 | 35138396 | C/T |
| 1 | 35162030 | T/C |
| 1 | 35214610 | C/T |
| 1 | 35272732 | G/C |
| 1 | 35311523 | G/A |
| 1 | 35325486 | C/T |
| 1 | 35407826 | G/A |
| 1 | 35473571 | G/C |
| 1 | 35486018 | G/A |
| 1 | 35559908 | G/C |
| 1 | 35613467 | C/T |
| 1 | 35675336 | A/T |
| 1 | 35696210 | T/C |
| 1 | 35711006 | C/T |
| 1 | 35723425 | A/G |
| 1 | 35753360 | G/A |
| 1 | 35772444 | C/T |
| 1 | 35820625 | G/A |
| 1 | 35828273 | A/G |
| 1 | 35828274 | C/T |
| 1 | 35828275 | C/T |
| 1 | 35828276 | C/T |
| 1 | 35965929 | T/C |
| 1 | 35988939 | C/G |
| 1 | 36005616 | T/C |
| 1 | 36097028 | G/A |
| 1 | 36120713 | G/A |
| 1 | 36123074 | G/C |
| 1 | 36249245 | A/G |
| 1 | 36265208 | G/T |
| 1 | 36290437 | A/T |
| 1 | 36328464 | A/C |
| 1 | 36351530 | T/A |
| 1 | 36379324 | C/T |
| 1 | 36391423 | C/T |
| 1 | 36402472 | C/T |
| 1 | 36438845 | A/G |
| 1 | 36498504 | C/T |
| 1 | 36516757 | C/G |
| 1 | 36528047 | C/T |
| 1 | 36551045 | C/T |
| 1 | 36581692 | C/T |
| 1 | 36653145 | G/C |
| 1 | 36669715 | T/C |
| 1 | 36737434 | C/A |
| 1 | 36749178 | G/A |
| 1 | 36781368 | A/G |
| 1 | 36775773 | A/C |
| 1 | 36795304 | T/C |
| 1 | 36859339 | A/T |
| 1 | 36882990 | G/A |
| 1 | 36948897 | G/A |
| 1 | 36978661 | T/C |
| 1 | 37018082 | G/C |
| 1 | 37049909 | C/T |
| 1 | 37052182 | C/T |
| 1 | 37111591 | G/A |
| 1 | 37134982 | T/C |
| 1 | 37149176 | C/T |
| 1 | 37171079 | G/A |
| 1 | 37189802 | C/T |
| 1 | 37231037 | C/T |
| 1 | 37244072 | G/A |
| 1 | 37268734 | G/A |
| 1 | 37292201 | T/C |
| 1 | 37305926 | A/G |
| 1 | 37350762 | G/A |
| 1 | 37387479 | A/G |
| 1 | 37430227 | G/A |
| 1 | 37595812 | A/C |
| 1 | 37657856 | A/G |
| 1 | 37662383 | T/G |
| 1 | 37708540 | T/G |
| 1 | 37735570 | A/G |
| 1 | 37746929 | T/C |
| 1 | 37753894 | C/T |
| 1 | 37771973 | G/A |
| 1 | 37809529 | A/G |
| 1 | 37869705 | C/G |
| 1 | 37871525 | G/A |
| 1 | 37890077 | T/C |
| 1 | 37910385 | G/A |
| 1 | 37926169 | G/A |
| 1 | 37960142 | C/T |
| 1 | 37975507 | G/A |
| 1 | 37989078 | G/A |
| 1 | 38014580 | C/T |
| 1 | 38033140 | C/A |
| 1 | 38105644 | C/T |
| 1 | 38152920 | G/A |
| 1 | 38170160 | G/C |
| 1 | 38220806 | C/T |
| 1 | 38227466 | G/A |
| 1 | 38269868 | G/T |
| 1 | 38283966 | C/T |
| 1 | 38327374 | G/A |
| 1 | 38395797 | C/T |
| 1 | 38447646 | C/T |
| 1 | 38546535 | C/T |
| 1 | 38619086 | C/T |
| 1 | 38637143 | A/G |
| 1 | 38662824 | C/T |
| 1 | 38662825 | C/A |
| 1 | 38662826 | C/A |
| 1 | 38662827 | C/A |
| 1 | 38948202 | A/C |
| 1 | 39162625 | T/C |
| 1 | 39170045 | G/A |
| 1 | 39373205 | G/A |
| 1 | 39378666 | C/T |
| 1 | 39435046 | G/A |
| 1 | 39588418 | C/T |
| 1 | 39617973 | T/A |
| 1 | 39621338 | G/C |
| 1 | 39632962 | C/G |
| 1 | 39680358 | G/A |
| 1 | 39704553 | C/T |
| 1 | 39783688 | G/A |
| 1 | 39862832 | A/T |
| 1 | 39866668 | T/G |
| 1 | 39935930 | G/A |
| 1 | 40035734 | G/C |
| 1 | 40068740 | C/T |
| 1 | 40086409 | G/A |
| 1 | 40124330 | G/C |
| 1 | 40139591 | A/G |
| 1 | 40198560 | G/T |
| 1 | 40203852 | C/T |
| 1 | 40234714 | C/G |
| 1 | 40261589 | C/A |
| 1 | 40296005 | T/C |
| 1 | 40300281 | A/G |
| 1 | 40355209 | A/G |
| 1 | 40361443 | C/T |
| 1 | 40390052 | G/T |
| 1 | 40414185 | G/C |
| 1 | 40471962 | C/T |
| 1 | 40513587 | C/T |
| 1 | 40516885 | C/A |
| 1 | 40532624 | C/T |
| 1 | 40577445 | A/G |
| 1 | 40592551 | A/T |
| 1 | 40602322 | G/A |
| 1 | 40635925 | A/C |
| 1 | 40685994 | T/C |
| 1 | 40713761 | T/C |
| 1 | 40727868 | C/G |
| 1 | 40771841 | A/G |
| 1 | 40807316 | C/T |
| 1 | 40839586 | G/A |
| 1 | 40862996 | G/A |
| 1 | 40915306 | C/G |
| 1 | 40941184 | G/A |
| 1 | 40964482 | G/T |
| 1 | 41006494 | G/C |
| 1 | 41048283 | T/C |
| 1 | 41102995 | T/C |
| 1 | 41120850 | G/A |
| 1 | 41141969 | C/A |
| 1 | 41188269 | G/A |
| 1 | 41222196 | C/T |
| 1 | 41232498 | C/A |
| 1 | 41272311 | C/T |
| 1 | 41279020 | C/T |
| 1 | 41307967 | C/A |
| 1 | 41312901 | G/A |
| 1 | 41317293 | T/G |
| 1 | 41348120 | C/T |
| 1 | 41360084 | A/G |
| 1 | 41378633 | C/T |
| 1 | 41407525 | A/G |
| 1 | 41449074 | T/G |
| 1 | 41479220 | A/G |
| 1 | 41496096 | C/T |
| 1 | 41516556 | C/T |
| 1 | 41562757 | G/A |
| 1 | 41568165 | G/A |
| 1 | 41596850 | G/A |
| 1 | 41638159 | C/T |
| 1 | 41759297 | C/G |
| 1 | 41781491 | G/A |
| 1 | 41801806 | A/G |
| 1 | 41841769 | G/A |
| 1 | 41865178 | A/G |
| 1 | 41912989 | T/C |
| 1 | 41957461 | C/G |
| 1 | 41962782 | C/T |
| 1 | 41990754 | G/A |
| 1 | 42003611 | C/T |
| 1 | 42011869 | C/A |
| 1 | 42136041 | T/C |
| 1 | 42332253 | T/G |
| 1 | 42381306 | A/G |
| 1 | 42408525 | G/A |
| 1 | 42452121 | C/T |
| 1 | 42464250 | T/G |
| 1 | 42537216 | G/A |
| 1 | 42552883 | A/G |
| 1 | 42576219 | G/A |
| 1 | 42602006 | A/T |
| 1 | 42608567 | A/G |
| 1 | 42630800 | G/A |
| 1 | 42663743 | A/G |
| 1 | 42715223 | G/A |
| 1 | 42748216 | C/T |
| 1 | 42780839 | T/A |
| 1 | 42789872 | T/C |
| 1 | 42818639 | G/A |
| 1 | 42866414 | G/C |
| 1 | 42875093 | G/A |
| 1 | 42881466 | C/T |
| 1 | 42918091 | A/C |
| 1 | 42955271 | C/G |
| 1 | 42958902 | A/G |
| 1 | 42961811 | T/G |
| 1 | 42967401 | A/G |
| 1 | 42967402 | A/G |
| 1 | 42967403 | A/G |
| 1 | 42967404 | A/G |
| 1 | 43026197 | C/T |
| 1 | 43034203 | G/C |
| 1 | 43062053 | C/T |
| 1 | 43101494 | G/A |
| 1 | 43105457 | T/C |
| 1 | 43123015 | C/T |
| 1 | 43142030 | T/C |
| 1 | 43178541 | T/G |
| 1 | 43195852 | A/T |
| 1 | 43218014 | A/G |
| 1 | 43224927 | A/T |
| 1 | 43248169 | A/C |
| 1 | 43249208 | T/C |
| 1 | 43260329 | A/C |
| 1 | 43268359 | G/T |
| 1 | 43299035 | C/A |
| 1 | 43300394 | G/A |
| 1 | 43327155 | C/T |
| 1 | 43341303 | A/G |
| 1 | 43355469 | A/G |
| 1 | 43380114 | C/T |
| 1 | 43407462 | T/G |
| 1 | 43415307 | A/C |
| 1 | 43439677 | G/A |
| 1 | 43452579 | C/A |
| 1 | 43470440 | T/A |
| 1 | 43505618 | A/G |
| 1 | 43516870 | A/C |
| 1 | 43540483 | T/G |
| 1 | 43544804 | T/C |
| 1 | 43576597 | T/G |
| 1 | 43585736 | G/T |
| 1 | 43591358 | A/G |
| 1 | 43607169 | T/C |
| 1 | 43628871 | G/T |
| 1 | 43635664 | C/T |
| 1 | 43652858 | T/C |
| 1 | 43662020 | G/A |
| 1 | 43686413 | T/A |
| 1 | 43729176 | T/C |
| 1 | 43730303 | T/G |
| 1 | 43759264 | C/T |
| 1 | 43767205 | T/C |
| 1 | 43783138 | T/C |
| 1 | 43797879 | T/G |
| 1 | 43807051 | G/A |
| 1 | 43810686 | A/C |
| 1 | 43844536 | C/A |
| 1 | 43858250 | G/A |
| 1 | 43891485 | C/G |
| 1 | 43917084 | A/G |
| 1 | 43967383 | C/T |
| 1 | 44006487 | C/A |
| 1 | 44032756 | G/C |
| 1 | 44037966 | A/G |
| 1 | 44040689 | G/A |
| 1 | 44094474 | G/T |
| 1 | 44100837 | T/C |
| 1 | 44104647 | G/A |
| 1 | 44139190 | C/T |
| 1 | 44155906 | A/G |
| 1 | 44166870 | T/C |
| 1 | 44187169 | A/C |
| 1 | 44193930 | C/T |
| 1 | 44227194 | C/T |
| 1 | 44253054 | T/A |
| 1 | 44255979 | A/G |
| 1 | 44280786 | A/G |
| 1 | 44304341 | G/A |
| 1 | 44310900 | G/A |
| 1 | 44342193 | T/A |
| 1 | 44351122 | G/T |
| 1 | 44392607 | C/T |
| 1 | 44419245 | C/T |
| 1 | 44424484 | A/T |
| 1 | 44431075 | A/G |
| 1 | 44450561 | T/C |
| 1 | 44476277 | G/A |
| 1 | 44484812 | C/A |
| 1 | 44496527 | A/C |
| 1 | 44511015 | G/A |
| 1 | 44521597 | G/A |
| 1 | 44568905 | C/T |
| 1 | 44583542 | G/A |
| 1 | 44588358 | C/T |
| 1 | 44613448 | C/T |
| 1 | 44615600 | G/A |
| 1 | 44669455 | C/A |
| 1 | 44671096 | C/T |
| 1 | 44707858 | G/A |
| 1 | 44725762 | G/A |
| 1 | 44734830 | A/C |
| 1 | 44758009 | G/T |
| 1 | 44760392 | G/T |
| 1 | 44774298 | C/T |
| 1 | 44811529 | A/G |
| 1 | 44816879 | G/A |
| 1 | 44834980 | G/A |
| 1 | 44848099 | C/T |
| 1 | 44853240 | G/A |
| 1 | 44871062 | C/T |
| 1 | 44905695 | G/A |
| 1 | 44936905 | G/T |
| 1 | 44950001 | C/T |
| 1 | 44985912 | C/T |
| 1 | 44987780 | G/A |
| 1 | 45056884 | T/C |
| 1 | 45059974 | C/T |
| 1 | 45069665 | C/A |
| 1 | 45127704 | C/T |
| 1 | 45141585 | G/A |
| 1 | 45164423 | C/T |
| 1 | 45195545 | G/A |
| 1 | 45219017 | A/T |
| 1 | 45223406 | C/T |
| 1 | 45253731 | T/C |
| 1 | 45256239 | G/C |
| 1 | 45271119 | A/G |
| 1 | 45319102 | G/A |
| 1 | 45323790 | T/C |
| 1 | 45340420 | G/A |
| 1 | 45374568 | C/T |
| 1 | 45379149 | A/G |
| 1 | 45409416 | A/T |
| 1 | 45445075 | A/G |
| 1 | 45446633 | A/G |
| 1 | 45460410 | A/G |
| 1 | 45506359 | A/C |
| 1 | 45514808 | G/A |
| 1 | 45516865 | T/A |
| 1 | 45559014 | C/T |
| 1 | 45567549 | G/A |
| 1 | 45576951 | G/A |
| 1 | 45613885 | G/A |
| 1 | 45621452 | C/T |
| 1 | 45640553 | G/T |
| 1 | 45657168 | A/G |
| 1 | 45685101 | G/A |
| 1 | 45693883 | C/T |
| 1 | 45717241 | G/T |
| 1 | 45721437 | T/G |
| 1 | 45735478 | A/G |
| 1 | 45753252 | G/A |
| 1 | 45769678 | T/C |
| 1 | 45780027 | G/A |
| 1 | 45795864 | G/A |
| 1 | 45811625 | G/A |
| 1 | 45814507 | C/T |
| 1 | 45848359 | C/T |
| 1 | 45859754 | C/T |
| 1 | 45886961 | G/A |
| 1 | 45912330 | C/T |
| 1 | 45926684 | T/C |
| 1 | 45933775 | G/A |
| 1 | 45942798 | T/C |
| 1 | 45984543 | A/G |
| 1 | 46007033 | A/G |
| 1 | 46017394 | T/C |
| 1 | 46021731 | G/A |
| 1 | 46029540 | C/T |
| 1 | 46059345 | G/A |
| 1 | 46083893 | A/C |
| 1 | 46130688 | C/T |
| 1 | 46135492 | C/G |
| 1 | 46145480 | A/G |
| 1 | 46154518 | C/T |
| 1 | 46183516 | C/T |
| 1 | 46211201 | A/G |
| 1 | 46214972 | A/C |
| 1 | 46244292 | T/C |
| 1 | 46273729 | G/A |
| 1 | 46274977 | G/C |
| 1 | 46292357 | C/T |
| 1 | 46329857 | T/C |
| 1 | 46331036 | A/G |
| 1 | 46351031 | A/G |
| 1 | 46378704 | T/C |
| 1 | 46407547 | C/T |
| 1 | 46420829 | A/C |
| 1 | 46444717 | A/G |
| 1 | 46452429 | T/A |
| 1 | 46470797 | T/C |
| 1 | 46512340 | G/A |
| 1 | 46523386 | C/T |
| 1 | 46548179 | C/T |
| 1 | 46564679 | G/T |
| 1 | 46565756 | C/T |
| 1 | 46602528 | G/T |
| 1 | 46607628 | G/T |
| 1 | 46627014 | T/C |
| 1 | 46633474 | G/C |
| 1 | 46653539 | G/A |
| 1 | 46690549 | G/T |
| 1 | 46700536 | A/G |
| 1 | 46721653 | G/A |
| 1 | 46752032 | G/A |
| 1 | 46761196 | T/G |
| 1 | 46772671 | C/T |
| 1 | 46824327 | G/A |
| 1 | 46832877 | A/T |
| 1 | 46859896 | C/T |
| 1 | 46872016 | C/T |
| 1 | 46887397 | C/T |
| 1 | 46915573 | G/A |
| 1 | 46930012 | C/T |
| 1 | 46965611 | C/A |
| 1 | 46977850 | A/C |
| 1 | 46999437 | G/T |
| 1 | 47023970 | G/T |
| 1 | 47067704 | G/A |
| 1 | 47073019 | G/A |
| 1 | 47110259 | C/A |
| 1 | 47152644 | C/T |
| 1 | 47220182 | C/A |
| 1 | 47255353 | T/C |
| 1 | 47294899 | G/A |
| 1 | 47296215 | C/T |
| 1 | 47313149 | A/T |
| 1 | 47349345 | C/T |
| 1 | 47359738 | C/T |
| 1 | 47380954 | A/C |
| 1 | 47423632 | C/T |
| 1 | 47434678 | G/A |
| 1 | 47436957 | G/A |
| 1 | 47464750 | C/A |
| 1 | 47475247 | G/A |
| 1 | 47516814 | A/G |
| 1 | 47538868 | C/A |
| 1 | 47564242 | C/T |
| 1 | 47578158 | G/A |
| 1 | 47607162 | T/G |
| 1 | 47613915 | A/G |
| 1 | 47648200 | C/T |
| 1 | 47662491 | C/T |
| 1 | 47678173 | C/A |
| 1 | 47715132 | A/G |
| 1 | 47729565 | G/A |
| 1 | 47772019 | G/A |
| 1 | 47841437 | G/C |
| 1 | 47846110 | C/T |
| 1 | 47922044 | G/A |
| 1 | 47963564 | C/T |
| 1 | 47967211 | G/A |
| 1 | 47976082 | G/T |
| 1 | 47978629 | C/T |
| 1 | 48021631 | G/C |
| 1 | 48043965 | G/A |
| 1 | 48056195 | G/A |
| 1 | 48076321 | C/T |
| 1 | 48088987 | C/T |
| 1 | 48094440 | C/T |
| 1 | 48137465 | A/C |
| 1 | 48160221 | G/A |
| 1 | 48171722 | G/A |
| 1 | 48181406 | A/G |
| 1 | 48209932 | G/A |
| 1 | 48214792 | T/C |
| 1 | 48225160 | G/A |
| 1 | 48297526 | G/T |
| 1 | 48312855 | G/A |
| 1 | 48312856 | G/A |
| 1 | 48312857 | G/A |
| 1 | 48312858 | G/A |
| 1 | 48362942 | G/A |
| 1 | 48376847 | C/T |
| 1 | 48412532 | T/C |
| 1 | 48447933 | G/T |
| 1 | 48472468 | T/C |
| 1 | 48476399 | C/T |
| 1 | 48480286 | T/C |
| 1 | 48495077 | C/T |
| 1 | 48510293 | T/C |
| 1 | 48518909 | G/A |
| 1 | 48544604 | A/G |
| 1 | 48574066 | C/T |
| 1 | 48600067 | T/C |
| 1 | 48604272 | T/A |
| 1 | 48645520 | T/G |
| 1 | 48659272 | T/G |
| 1 | 48672266 | G/A |
| 1 | 48684175 | C/T |
| 1 | 48694509 | G/T |
| 1 | 48707123 | A/C |
| 1 | 48745924 | A/G |
| 1 | 48753019 | T/C |
| 1 | 48772379 | A/T |
| 1 | 48780091 | T/G |
| 1 | 48810136 | C/T |
| 1 | 48848714 | G/A |
| 1 | 48892300 | G/C |
| 1 | 48906632 | T/C |
| 1 | 48929755 | A/C |
| 1 | 48934754 | C/T |
| 1 | 48939049 | T/C |
| 1 | 48963685 | A/G |
| 1 | 48968350 | G/A |
| 1 | 49015243 | A/G |
| 1 | 49016436 | T/C |
| 1 | 49031930 | G/A |
| 1 | 49050954 | T/A |
| 1 | 49103270 | G/A |
| 1 | 49105468 | A/C |
| 1 | 49132481 | G/A |
| 1 | 49140613 | G/A |
| 1 | 49171586 | C/A |
| 1 | 49213937 | A/G |
| 1 | 49239716 | T/C |
| 1 | 49257195 | G/C |
| 1 | 49288362 | A/G |
| 1 | 49293960 | G/C |
| 1 | 49296697 | A/G |
| 1 | 49344438 | T/C |
| 1 | 49357496 | C/T |
| 1 | 49366215 | G/A |
| 1 | 49405226 | G/C |
| 1 | 49419142 | G/C |
| 1 | 49443759 | A/G |
| 1 | 49456024 | G/A |
| 1 | 49492775 | G/A |
| 1 | 49505734 | A/C |
| 1 | 49542616 | G/A |
| 1 | 49543743 | G/T |
| 1 | 49583687 | C/T |
| 1 | 49596310 | G/A |
| 1 | 49625740 | G/A |
| 1 | 49629220 | C/T |
| 1 | 49671621 | G/A |
| 1 | 49680786 | C/G |
| 1 | 49709756 | T/C |
| 1 | 49733110 | A/T |
| 1 | 49741187 | G/T |
| 1 | 49756352 | T/G |
| 1 | 49775776 | T/C |
| 1 | 49817830 | G/A |
| 1 | 49837122 | C/T |
| 1 | 49859700 | G/T |
| 1 | 49879755 | G/A |
| 1 | 49885020 | G/A |
| 1 | 49892947 | A/G |
| 1 | 49898880 | G/A |
| 1 | 49920844 | G/A |
| 1 | 49940119 | G/A |
| 1 | 49978751 | G/T |
| 1 | 50000135 | T/G |
| 1 | 50013931 | G/A |
| 1 | 50040580 | T/G |
| 1 | 50074011 | A/G |
| 1 | 50088161 | G/A |
| 1 | 50104007 | G/A |
| 1 | 50123793 | T/C |
| 1 | 50136628 | A/T |
| 1 | 50189423 | A/G |
| 1 | 50192772 | T/C |
| 1 | 50220353 | G/A |
| 1 | 50262600 | T/G |
| 1 | 50281628 | A/G |
| 1 | 50294690 | C/A |
| 1 | 50312221 | C/T |
| 1 | 50332285 | T/C |
| 1 | 50364665 | C/G |
| 1 | 50375100 | A/G |
| 1 | 50382431 | T/A |
| 1 | 50411304 | A/G |
| 1 | 50413137 | C/T |
| 1 | 50431732 | A/G |
| 1 | 50434435 | A/G |
| 1 | 50466531 | G/A |
| 1 | 50472552 | A/G |
| 1 | 50496108 | G/A |
| 1 | 50503186 | G/A |
| 1 | 50533180 | C/T |
| 1 | 50547839 | C/T |
| 1 | 50558440 | A/C |
| 1 | 50564666 | C/G |
| 1 | 50588598 | G/A |
| 1 | 50605143 | A/C |
| 1 | 50612070 | G/A |
| 1 | 50672297 | C/T |
| 1 | 50673541 | T/C |
| 1 | 50705077 | G/A |
| 1 | 50710997 | G/A |
| 1 | 50730924 | G/A |
| 1 | 50786043 | A/G |
| 1 | 50799534 | A/G |
| 1 | 50812511 | G/A |
| 1 | 50823121 | T/C |
| 1 | 50846119 | A/T |
| 1 | 50857594 | A/T |
| 1 | 50872023 | A/T |
| 1 | 50884787 | G/A |
| 1 | 50906608 | G/T |
| 1 | 50914622 | C/T |
| 1 | 50966949 | T/C |
| 1 | 50974003 | C/T |
| 1 | 50992162 | A/G |
| 1 | 51002943 | A/G |
| 1 | 51018586 | G/A |
| 1 | 51037401 | A/G |
| 1 | 51059582 | C/T |
| 1 | 51067022 | A/G |
| 1 | 51068041 | A/G |
| 1 | 51098653 | A/C |
| 1 | 51117186 | T/C |
| 1 | 51125780 | T/A |
| 1 | 51165913 | G/A |
| 1 | 51174750 | C/T |
| 1 | 51180007 | T/C |
| 1 | 51223270 | A/G |
| 1 | 51227857 | A/G |
| 1 | 51243859 | T/C |
| 1 | 51252859 | C/T |
| 1 | 51279758 | T/C |
| 1 | 51282852 | T/A |
| 1 | 51282853 | C/T |
| 1 | 51282854 | C/T |
| 1 | 51282855 | C/T |
| 1 | 51336018 | T/C |
| 1 | 51369862 | T/G |
| 1 | 51392714 | A/G |
| 1 | 51411708 | T/A |
| 1 | 51422016 | A/C |
| 1 | 51428573 | G/C |
| 1 | 51473729 | G/A |
| 1 | 51495545 | G/A |
| 1 | 51500233 | T/C |
| 1 | 51525371 | C/A |
| 1 | 51565349 | T/C |
| 1 | 51592826 | G/A |
| 1 | 51600545 | G/A |
| 1 | 51635176 | G/A |
| 1 | 51674308 | G/A |
| 1 | 51679004 | G/A |
| 1 | 51696206 | C/A |
| 1 | 51743234 | C/T |
| 1 | 51744549 | G/A |
| 1 | 51765298 | G/A |
| 1 | 51805190 | C/T |
| 1 | 51821937 | G/A |
| 1 | 51827986 | C/T |
| 1 | 51867582 | G/A |
| 1 | 51889058 | G/A |
| 1 | 51895547 | A/G |
| 1 | 51913786 | G/T |
| 1 | 51941134 | G/A |
| 1 | 51981270 | G/A |
| 1 | 51987731 | G/C |
| 1 | 52042669 | G/A |
| 1 | 52059341 | A/C |
| 1 | 52081631 | T/A |
| 1 | 52092009 | C/T |
| 1 | 52111710 | C/T |
| 1 | 52125658 | G/A |
| 1 | 52181585 | G/A |
| 1 | 52186382 | G/A |
| 1 | 52208215 | G/A |
| 1 | 52247544 | C/T |
| 1 | 52250071 | C/A |
| 1 | 52265446 | C/T |
| 1 | 52298612 | G/A |
| 1 | 52340402 | T/A |
| 1 | 52375271 | T/C |
| 1 | 52385840 | A/G |
| 1 | 52415729 | G/A |
| 1 | 52424796 | C/T |
| 1 | 52443789 | C/T |
| 1 | 52469815 | G/A |
| 1 | 52509879 | C/T |
| 1 | 52549115 | A/G |
| 1 | 52569983 | C/T |
| 1 | 52596477 | G/A |
| 1 | 52617928 | G/A |
| 1 | 52642185 | G/A |
| 1 | 52646416 | G/A |
| 1 | 52684186 | C/T |
| 1 | 52719568 | A/T |
| 1 | 52757007 | T/G |
| 1 | 52764874 | T/C |
| 1 | 52828596 | T/C |
| 1 | 52832614 | G/A |
| 1 | 52876109 | A/G |
| 1 | 52914014 | A/G |
| 1 | 52940829 | G/A |
| 1 | 52950682 | C/T |
| 1 | 52978241 | C/T |
| 1 | 52987787 | G/A |
| 1 | 53027346 | C/T |
| 1 | 53055706 | A/T |
| 1 | 53080747 | G/A |
| 1 | 53095184 | G/A |
| 1 | 53103037 | C/A |
| 1 | 53118440 | A/C |
| 1 | 53142879 | C/A |
| 1 | 53145385 | G/A |
| 1 | 53186324 | G/A |
| 1 | 53191091 | G/A |
| 1 | 53219588 | G/A |
| 1 | 53225995 | C/T |
| 1 | 53240759 | G/A |
| 1 | 53273667 | G/A |
| 1 | 53285858 | C/A |
| 1 | 53324774 | G/A |
| 1 | 53366899 | C/A |
| 1 | 53386191 | G/A |
| 1 | 53387417 | T/C |
| 1 | 53400761 | A/G |
| 1 | 53434626 | C/T |
| 1 | 53457500 | C/A |
| 1 | 53482546 | C/T |
| 1 | 53487670 | G/T |
| 1 | 53502380 | G/T |
| 1 | 53520487 | G/C |
| 1 | 53537917 | C/T |
| 1 | 53554670 | G/A |
| 1 | 53564221 | C/T |
| 1 | 53586353 | A/C |
| 1 | 53587957 | C/G |
| 1 | 53614822 | G/A |
| 1 | 53650163 | T/G |
| 1 | 53682749 | A/G |
| 1 | 53720308 | G/A |
| 1 | 53722586 | A/T |
| 1 | 53730515 | T/C |
| 1 | 53758612 | A/C |
| 1 | 53764045 | T/C |
| 1 | 53803890 | G/A |
| 1 | 53816699 | T/C |
| 1 | 53824608 | C/T |
| 1 | 53843046 | C/G |
| 1 | 53852275 | G/A |
| 1 | 53885468 | C/A |
| 1 | 53905214 | G/T |
| 1 | 53912302 | C/T |
| 1 | 53952582 | G/C |
| 1 | 53987255 | C/G |
| 1 | 53994537 | G/A |
| 1 | 54006418 | A/G |
| 1 | 54013419 | G/A |
| 1 | 54034846 | G/C |
| 1 | 54061403 | G/A |
| 1 | 54067931 | A/G |
| 1 | 54094633 | T/C |
| 1 | 54098269 | C/T |
| 1 | 54130349 | A/C |
| 1 | 54131565 | G/C |
| 1 | 54153788 | C/G |
| 1 | 54163194 | C/T |
| 1 | 54185695 | T/C |
| 1 | 54191320 | A/G |
| 1 | 54234037 | T/G |
| 1 | 34242028 | A/C |
| 1 | 54249271 | T/A |
| 1 | 54280244 | T/C |
| 1 | 54317000 | C/A |
| 1 | 54320884 | G/C |
| 1 | 54330200 | T/C |
| 1 | 54341896 | T/C |
| 1 | 54362894 | C/T |
| 1 | 54379572 | C/A |
| 1 | 54406491 | T/C |
| 1 | 54408939 | G/A |
| 1 | 54426780 | A/G |
| 1 | 54455366 | T/C |
| 1 | 54468849 | C/T |
| 1 | 54487248 | C/G |
| 1 | 54495107 | G/T |
| 1 | 54516412 | T/C |
| 1 | 54529297 | A/G |
| 1 | 54540736 | A/T |
| 1 | 54544147 | A/G |
| 1 | 54589281 | C/T |
| 1 | 54597837 | A/G |
| 1 | 54602595 | T/C |
| 1 | 54608299 | T/C |
| 1 | 54635919 | T/C |
| 1 | 54652358 | A/G |
| 1 | 54684701 | C/T |
| 1 | 54692099 | A/G |
| 1 | 54724515 | A/G |
| 1 | 54740315 | T/A |
| 1 | 54751474 | G/A |
| 1 | 54791571 | C/T |
| 1 | 54812006 | C/A |
| 1 | 54832462 | C/T |
| 1 | 54859499 | T/G |
| 1 | 54865638 | C/G |
| 1 | 54904321 | G/T |
| 1 | 54925678 | T/C |
| 1 | 54932641 | G/A |
| 1 | 54972180 | T/C |
| 1 | 54973232 | T/C |
| 1 | 54991126 | A/C |
| 1 | 55019701 | T/A |
| 1 | 55035636 | A/C |
| 1 | 55039819 | T/C |
| 1 | 55064817 | G/A |
| 1 | 55087481 | G/C |
| 1 | 55110551 | T/C |
| 1 | 55148017 | G/A |
| 1 | 55149499 | G/A |
| 1 | 55174107 | G/A |
| 1 | 55192338 | A/T |
| 1 | 55201559 | G/T |
| 1 | 55253847 | G/T |
| 1 | 55259611 | A/T |
| 1 | 55265510 | A/T |
| 1 | 55281163 | C/A |
| 1 | 55295231 | C/A |
| 1 | 55338801 | G/A |
| 1 | 55346118 | A/G |
| 1 | 55375664 | G/A |
| 1 | 55384502 | C/A |
| 1 | 55396591 | C/T |
| 1 | 55427451 | A/T |
| 1 | 55435692 | G/A |
| 1 | 55458868 | A/G |
| 1 | 55465356 | C/T |
| 1 | 55470387 | A/G |
| 1 | 55520102 | G/C |
| 1 | 55538297 | C/T |
| 1 | 55560606 | G/A |
| 1 | 55564106 | G/C |
| 1 | 55595592 | A/G |
| 1 | 55617424 | G/A |
| 1 | 55624793 | A/G |
| 1 | 55638793 | G/A |
| 1 | 55669175 | G/A |
| 1 | 55671414 | A/T |
| 1 | 55683933 | T/C |
| 1 | 55737721 | C/A |
| 1 | 55740071 | A/T |
| 1 | 55741830 | T/A |
| 1 | 55796410 | C/T |
| 1 | 55827927 | A/T |
| 1 | 55854893 | C/T |
| 1 | 55875340 | A/T |
| 1 | 55894680 | G/A |
| 1 | 55896295 | C/T |
| 1 | 55924276 | A/G |
| 1 | 55967676 | A/G |
| 1 | 55979419 | C/T |
| 1 | 55982818 | C/T |
| 1 | 55997577 | A/G |
| 1 | 56015054 | C/T |
| 1 | 56067599 | C/T |
| 1 | 56098585 | C/A |
| 1 | 56100921 | C/A |
| 1 | 56110336 | A/C |
| 1 | 56148636 | G/T |
| 1 | 56154062 | A/G |
| 1 | 56166748 | C/T |
| 1 | 56210920 | G/A |
| 1 | 56222382 | C/T |
| 1 | 56238369 | G/A |
| 1 | 56263021 | C/A |
| 1 | 56275280 | T/A |
| 1 | 56308021 | C/T |
| 1 | 56310195 | T/A |
| 1 | 56311746 | A/G |
| 1 | 56344948 | A/G |
| 1 | 56368104 | C/T |
| 1 | 56393412 | C/G |
| 1 | 56422889 | G/A |
| 1 | 56429676 | A/G |
| 1 | 56429677 | T/G |
| 1 | 56442503 | A/G |
| 1 | 56463525 | T/A |
| 1 | 56505675 | C/A |
| 1 | 56532895 | A/G |
| 1 | 56541560 | A/G |
| 1 | 56570822 | C/T |
| 1 | 56583375 | A/T |
| 1 | 56596239 | C/T |
| 1 | 56617841 | A/G |
| 1 | 56664464 | T/C |
| 1 | 56667420 | C/A |
| 1 | 56683999 | C/A |
| 1 | 56700546 | T/A |
| 1 | 36742164 | G/A |
| 1 | 56758470 | T/A |
| 1 | 56768386 | T/A |
| 1 | 56804011 | A/G |
| 1 | 56821538 | C/T |
| 1 | 56836475 | A/G |
| 1 | 56869849 | C/A |
| 1 | 56902594 | T/C |
| 1 | 56904065 | C/T |
| 1 | 56936979 | C/T |
| 1 | 56946625 | C/T |
| 1 | 56948934 | G/A |
| 1 | 56979613 | T/C |
| 1 | 57000806 | G/A |
| 1 | 57019990 | T/C |
| 1 | 57050198 | G/A |
| 1 | 57058157 | C/T |
| 1 | 57061313 | A/G |
| 1 | 57086036 | G/A |
| 1 | 57104650 | T/C |
| 1 | 57118660 | C/G |
| 1 | 57121179 | A/G |
| 1 | 57122853 | T/A |
| 1 | 57155433 | G/A |
| 1 | 57165979 | A/T |
| 1 | 57199394 | C/T |
| 1 | 57202688 | G/A |
| 1 | 57223053 | C/T |
| 1 | 57254678 | A/G |
| 1 | 57263012 | G/A |
| 1 | 57275232 | A/T |
| 1 | 57300385 | G/A |
| 1 | 57319946 | T/G |
| 1 | 57350410 | C/T |
| 1 | 57364236 | A/T |
| 1 | 57375742 | G/A |
| 1 | 57392286 | G/T |
| 1 | 57416393 | C/T |
| 1 | 57434456 | G/C |
| 1 | 57467947 | T/G |
| 1 | 57493259 | G/A |
| 1 | 57505355 | G/A |
| 1 | 57521020 | G/A |
| 1 | 57532743 | T/G |
| 1 | 57540504 | A/G |
| 1 | 57584376 | C/A |
| 1 | 57588803 | G/A |
| 1 | 57606851 | G/C |
| 1 | 57643071 | C/T |
| 1 | 57647280 | C/A |
| 1 | 57670512 | G/A |
| 1 | 57675634 | C/T |
| 1 | 57712084 | G/A |
| 1 | 57734132 | T/C |
| 1 | 57745956 | G/A |
| 1 | 57776047 | C/T |
| 1 | 57790185 | G/T |
| 1 | 57794967 | G/A |
| 1 | 57830771 | G/A |
| 1 | 57843176 | T/C |
| 1 | 57846373 | A/G |
| 1 | 57873859 | C/T |
| 1 | 57922021 | A/T |
| 1 | 57924733 | G/A |
| 1 | 58009301 | C/T |
| 1 | 58046977 | G/A |
| 1 | 38049240 | G/A |
| 1 | 58059429 | G/A |
| 1 | 58081171 | G/A |
| 1 | 58144209 | C/G |
| 1 | 58146276 | G/A |
| 1 | 58172465 | C/T |
| 1 | 58181808 | A/G |
| 1 | 58182238 | C/T |
| 1 | 58229882 | G/A |
| 1 | 58240901 | C/T |
| 1 | 58264660 | C/T |
| 1 | 58294142 | G/A |
| 1 | 58297115 | G/A |
| 1 | 58329947 | T/C |
| 1 | 58334916 | T/C |
| 1 | 58357049 | T/C |
| 1 | 58363190 | G/A |
| 1 | 58392569 | T/G |
| 1 | 58409353 | T/A |
| 1 | 58411679 | C/T |
| 1 | 58446606 | T/C |
| 1 | 58499309 | G/A |
| 1 | 58511380 | A/G |
| 1 | 58525597 | A/G |
| 1 | 58532449 | T/A |
| 1 | 58545208 | T/G |
| 1 | 58560150 | C/G |
| 1 | 58579107 | C/T |
| 1 | 58604662 | T/C |
| 1 | 58606756 | A/C |
| 1 | 58625750 | A/T |
| 1 | 58679753 | C/T |
| 1 | 58681967 | T/C |
| 1 | 58702864 | G/C |
| 1 | 58720345 | G/T |
| 1 | 58762090 | A/C |
| 1 | 58783176 | G/A |
| 1 | 58787997 | C/T |
| 1 | 58817669 | A/C |
| 1 | 58830213 | A/T |
| 1 | 58854935 | C/T |
| 1 | 58869669 | C/G |
| 1 | 58899042 | T/A |
| 1 | 58949459 | G/C |
| 1 | 58954099 | T/A |
| 1 | 58955931 | C/T |
| 1 | 58992832 | G/A |
| 1 | 59021364 | A/G |
| 1 | 59040066 | T/A |
| 1 | 59044605 | C/G |
| 1 | 59121094 | A/C |
| 1 | 59131513 | A/G |
| 1 | 59139975 | C/T |
| 1 | 59187627 | A/C |
| 1 | 59190769 | G/A |
| 1 | 59191867 | A/G |
| 1 | 59227197 | T/C |
| 1 | 59237505 | G/C |
| 1 | 59253162 | C/A |
| 1 | 59297472 | G/A |
| 1 | 59303454 | G/A |
| 1 | 59325498 | G/T |
| 1 | 59330823 | A/G |
| 1 | 59352858 | T/C |
| 1 | 59361404 | G/T |
| 1 | 59372513 | G/A |
| 1 | 59428911 | A/C |
| 1 | 59436196 | T/C |
| 1 | 59448908 | A/C |
| 1 | 59474589 | A/C |
| 1 | 59477849 | C/T |
| 1 | 59500993 | C/A |
| 1 | 59513988 | A/G |
| 1 | 59520888 | C/T |
| 1 | 59526369 | T/C |
| 1 | 59550606 | A/T |
| 1 | 59583780 | T/C |
| 1 | 59586794 | T/A |
| 1 | 59612555 | T/C |
| 1 | 59633405 | A/G |
| 1 | 59640173 | G/A |
| 1 | 59669046 | A/T |
| 1 | 59673847 | C/T |
| 1 | 59701403 | T/G |
| 1 | 59711352 | A/G |
| 1 | 59743691 | G/A |
| 1 | 59787635 | T/C |
| 1 | 59791321 | G/A |
| 1 | 59825837 | C/T |
| 1 | 59868895 | A/G |
| 1 | 59870593 | A/G |
| 1 | 59902214 | G/A |
| 1 | 59922275 | G/A |
| 1 | 59938436 | G/A |
| 1 | 59947242 | A/G |
| 1 | 59955152 | A/T |
| 1 | 59975244 | T/C |
| 1 | 59978017 | T/G |
| 1 | 60022554 | A/T |
| 1 | 60024532 | T/C |
| 1 | 60032797 | T/C |
| 1 | 60057577 | G/A |
| 1 | 60065944 | C/A |
| 1 | 60098734 | G/C |
| 1 | 60109540 | T/C |
| 1 | 60130825 | T/A |
| 1 | 60151750 | G/A |
| 1 | 60172289 | T/G |
| 1 | 60227260 | C/T |
| 1 | 60233991 | G/C |
| 1 | 60243279 | C/A |
| 1 | 60254820 | T/A |
| 1 | 60286103 | C/T |
| 1 | 60324589 | G/A |
| 1 | 60349964 | C/A |
| 1 | 60351584 | C/T |
| 1 | 60372886 | G/A |
| 1 | 60381233 | T/C |
| 1 | 60391701 | C/T |
| 1 | 60410644 | C/A |
| 1 | 60430125 | T/C |
| 1 | 60431247 | C/G |
| 1 | 60471681 | C/T |
| 1 | 60481050 | C/T |
| 1 | 60504020 | A/G |
| 1 | 60516308 | A/G |
| 1 | 60540393 | G/A |
| 1 | 60581580 | G/A |
| 1 | 60594953 | A/G |
| 1 | 60617079 | G/A |
| 1 | 60622943 | G/A |
| 1 | 60631279 | T/C |
| 1 | 60661319 | G/C |
| 1 | 60663393 | A/C |
| 1 | 60699320 | A/G |
| 1 | 60708642 | C/G |
| 1 | 60722637 | C/T |
| 1 | 60728050 | T/G |
| 1 | 60754442 | A/C |
| 1 | 60784576 | A/T |
| 1 | 60802927 | T/C |
| 1 | 60858042 | C/T |
| 1 | 60911880 | A/T |
| 1 | 60929175 | T/C |
| 1 | 60930585 | T/A |
| 1 | 60977486 | C/T |
| 1 | 60995037 | T/G |
| 1 | 61038984 | A/C |
| 1 | 61048673 | G/A |
| 1 | 61056581 | C/G |
| 1 | 61081721 | G/A |
| 1 | 61093882 | G/T |
| 1 | 61134734 | A/G |
| 1 | 61151409 | C/T |
| 1 | 61184182 | C/T |
| 1 | 61218941 | T/C |
| 1 | 61245721 | A/G |
| 1 | 61267483 | A/G |
| 1 | 61275480 | C/T |
| 1 | 61294756 | C/T |
| 1 | 61318088 | G/A |
| 1 | 61324034 | A/T |
| 1 | 61337931 | G/C |
| 1 | 61352263 | T/C |
| 1 | 61392723 | G/C |
| 1 | 61414546 | T/C |
| 1 | 61460641 | T/G |
| 1 | 61473135 | T/G |
| 1 | 61491955 | G/A |
| 1 | 61504111 | T/C |
| 1 | 61532616 | C/T |
| 1 | 61575850 | T/C |
| 1 | 61581309 | A/G |
| 1 | 61595658 | T/C |
| 1 | 61607235 | C/T |
| 1 | 61620713 | G/A |
| 1 | 61662946 | G/A |
| 1 | 61674501 | G/A |
| 1 | 61683190 | T/C |
| 1 | 61701577 | C/T |
| 1 | 61736196 | A/C |
| 1 | 61752667 | A/G |
| 1 | 61768861 | G/A |
| 1 | 61793508 | G/T |
| 1 | 61795333 | C/T |
| 1 | 61822791 | T/C |
| 1 | 61825490 | A/G |
| 1 | 61830690 | G/A |
| 1 | 61849181 | G/A |
| 1 | 61861570 | G/T |
| 1 | 61898332 | C/T |
| 1 | 61949749 | G/A |
| 1 | 61952655 | A/C |
| 1 | 61989503 | C/T |
| 1 | 62058964 | T/C |
| 1 | 62091338 | C/T |
| 1 | 62114974 | A/G |
| 1 | 62132017 | T/C |
| 1 | 62141201 | A/G |
| 1 | 62165608 | G/A |
| 1 | 62254824 | T/C |
| 1 | 62304086 | A/G |
| 1 | 62330285 | G/A |
| 1 | 62337929 | T/C |
| 1 | 62382060 | A/G |
| 1 | 62395187 | C/T |
| 1 | 62410423 | C/T |
| 1 | 62429614 | G/A |
| 1 | 62451610 | T/C |
| 1 | 62460810 | C/T |
| 1 | 62464542 | C/T |
| 1 | 62506691 | C/T |
| 1 | 62522955 | G/A |
| 1 | 62577820 | G/T |
| 1 | 62581671 | C/T |
| 1 | 62675359 | A/G |
| 1 | 62680112 | T/G |
| 1 | 62734566 | A/G |
| 1 | 62740645 | T/C |
| 1 | 62784860 | C/T |
| 1 | 62807977 | T/C |
| 1 | 62809204 | C/G |
| 1 | 62842870 | T/A |
| 1 | 62845038 | A/G |
| 1 | 62877333 | C/T |
| 1 | 62894911 | A/T |
| 1 | 62930668 | A/G |
| 1 | 62945455 | A/G |
| 1 | 62970758 | T/G |
| 1 | 63020870 | C/T |
| 1 | 63047885 | A/G |
| 1 | 63076359 | T/C |
| 1 | 63089137 | A/T |
| 1 | 63097218 | T/C |
| 1 | 63130617 | T/C |
| 1 | 63151608 | C/T |
| 1 | 63162004 | T/C |
| 1 | 63198307 | A/G |
| 1 | 63221595 | G/A |
| 1 | 63251027 | G/A |
| 1 | 63296757 | C/T |
| 1 | 63302727 | A/G |
| 1 | 63321047 | T/A |
| 1 | 63349637 | C/G |
| 1 | 63357829 | G/A |
| 1 | 63388714 | T/C |
| 1 | 63391788 | A/G |
| 1 | 63393734 | G/C |
| 1 | 63426165 | G/A |
| 1 | 63444095 | C/G |
| 1 | 63462304 | G/A |
| 1 | 63474659 | G/A |
| 1 | 63503872 | G/A |
| 1 | 63533603 | C/T |
| 1 | 63535346 | G/A |
| 1 | 63558345 | T/C |
| 1 | 63636392 | T/C |
| 1 | 63676401 | G/A |
| 1 | 63724332 | C/A |
| 1 | 63751890 | G/A |
| 1 | 63788144 | C/T |
| 1 | 63814111 | A/G |
| 1 | 63832402 | C/T |
| 1 | 63845863 | C/T |
| 1 | 63889596 | T/G |
| 1 | 63900741 | A/G |
| 1 | 63916224 | C/G |
| 1 | 63940702 | G/A |
| 1 | 63942913 | G/A |
| 1 | 63976682 | G/A |
| 1 | 63998265 | A/T |
| 1 | 64038722 | G/A |
| 1 | 64072742 | G/A |
| 1 | 64103772 | G/A |
| 1 | 64128222 | G/A |
| 1 | 64143134 | C/T |
| 1 | 64191834 | A/G |
| 1 | 64197042 | G/T |
| 1 | 64206653 | T/C |
| 1 | 64216601 | T/A |
| 1 | 64232729 | C/T |
| 1 | 64258257 | T/C |
| 1 | 64262026 | A/G |
| 1 | 64272616 | A/G |
| 1 | 64311830 | G/T |
| 1 | 64321650 | C/G |
| 1 | 64340233 | G/A |
| 1 | 64351095 | A/C |
| 1 | 64408214 | C/T |
| 1 | 64411763 | A/G |
| 1 | 64438243 | G/A |
| 1 | 64462241 | C/T |
| 1 | 64498575 | C/A |
| 1 | 64525641 | C/T |
| 1 | 64548701 | C/T |
| 1 | 64550203 | T/C |
| 1 | 64562749 | A/G |
| 1 | 64566040 | C/A |
| 1 | 64594106 | A/T |
| 1 | 64598191 | C/A |
| 1 | 64636232 | T/G |
| 1 | 64654125 | G/A |
| 1 | 64670965 | C/G |
| 1 | 64681705 | G/A |
| 1 | 64686683 | C/T |
| 1 | 64714316 | T/C |
| 1 | 64768082 | T/C |
| 1 | 64790719 | C/T |
| 1 | 64800407 | G/C |
| 1 | 64830848 | C/A |
| 1 | 64865311 | T/G |
| 1 | 64873174 | A/G |
| 1 | 64892675 | T/C |
| 1 | 64909587 | G/C |
| 1 | 64926214 | G/A |
| 1 | 64934656 | G/T |
| 1 | 64957581 | A/G |
| 1 | 64981260 | T/C |
| 1 | 65020279 | G/A |
| 1 | 65043076 | T/C |
| 1 | 65086899 | T/A |
| 1 | 65087223 | C/T |
| 1 | 65087744 | T/C |
| 1 | 65120914 | T/C |
| 1 | 65127628 | G/A |
| 1 | 65149241 | C/G |
| 1 | 65166950 | G/A |
| 1 | 65197365 | A/G |
| 1 | 65199847 | A/T |
| 1 | 65239297 | C/T |
| 1 | 65261450 | C/T |
| 1 | 65263399 | A/G |
| 1 | 65281015 | G/A |
| 1 | 65311735 | T/A |
| 1 | 65328952 | G/C |
| 1 | 65348093 | C/G |
| 1 | 65364899 | T/G |
| 1 | 65390167 | C/A |
| 1 | 65400112 | T/A |
| 1 | 65425985 | G/T |
| 1 | 65430095 | G/T |
| 1 | 65475536 | A/C |
| 1 | 65479412 | C/T |
| 1 | 65499145 | C/T |
| 1 | 65546441 | A/G |
| 1 | 65563578 | G/A |
| 1 | 65568326 | G/A |
| 1 | 65581450 | G/A |
| 1 | 65610167 | C/T |
| 1 | 65655736 | T/G |
| 1 | 65665638 | T/C |
| 1 | 65676311 | G/C |
| 1 | 65688764 | A/T |
| 1 | 65711555 | C/A |
| 1 | 65729292 | C/A |
| 1 | 65730660 | T/C |
| 1 | 65771695 | T/C |
| 1 | 65801347 | G/A |
| 1 | 65817275 | C/T |
| 1 | 65823586 | G/C |
| 1 | 65836340 | C/G |
| 1 | 65865826 | T/C |
| 1 | 65870341 | G/T |
| 1 | 65889653 | G/A |
| 1 | 65907809 | G/A |
| 1 | 65911268 | T/A |
| 1 | 65951251 | T/C |
| 1 | 65977168 | A/G |
| 1 | 65999248 | C/A |
| 1 | 66002607 | A/G |
| 1 | 66008619 | C/A |
| 1 | 66038518 | C/G |
| 1 | 66078244 | G/A |
| 1 | 66081789 | A/G |
| 1 | 66090171 | C/T |
| 1 | 66094522 | G/C |
| 1 | 66136138 | C/T |
| 1 | 66170644 | G/A |
| 1 | 66184075 | G/A |
| 1 | 66215505 | G/A |
| 1 | 66296199 | C/T |
| 1 | 66306836 | G/A |
| 1 | 66338757 | T/A |
| 1 | 66361814 | C/A |
| 1 | 66374950 | T/C |
| 1 | 66430910 | A/G |
| 1 | 66440475 | C/T |
| 1 | 66451625 | T/C |
| 1 | 66468246 | T/G |
| 1 | 66498709 | A/G |
| 1 | 66521887 | G/A |
| 1 | 66543734 | A/G |
| 1 | 66566720 | A/G |
| 1 | 66619756 | C/A |
| 1 | 66630149 | A/G |
| 1 | 66661752 | G/A |
| 1 | 66681265 | A/G |
| 1 | 66698543 | A/T |
| 1 | 66747325 | C/T |
| 1 | 66762864 | T/C |
| 1 | 66809065 | G/A |
| 1 | 66813534 | A/G |
| 1 | 66831557 | C/G |
| 1 | 66886790 | C/T |
| 1 | 66913149 | G/A |
| 1 | 66945442 | T/A |
| 1 | 66958622 | G/C |
| 1 | 66966350 | G/A |
| 1 | 66990041 | G/T |
| 1 | 66996997 | T/A |
| 1 | 67041473 | T/G |
| 1 | 67077809 | T/C |
| 1 | 67099495 | A/C |
| 1 | 67113280 | G/A |
| 1 | 67114615 | C/T |
| 1 | 67151448 | A/G |
| 1 | 67195710 | T/G |
| 1 | 67204447 | G/A |
| 1 | 67213499 | A/G |
| 1 | 67258724 | A/G |
| 1 | 67269724 | T/A |
| 1 | 67297993 | G/A |
| 1 | 67333122 | C/A |
| 1 | 67348939 | C/A |
| 1 | 67351585 | C/G |
| 1 | 67352804 | G/C |
| 1 | 67380023 | G/T |
| 1 | 67398810 | C/T |
| 1 | 67411440 | C/A |
| 1 | 67441776 | C/T |
| 1 | 67443906 | T/C |
| 1 | 67486706 | C/A |
| 1 | 67501983 | C/T |
| 1 | 67511411 | T/A |
| 1 | 67583248 | A/G |
| 1 | 67603322 | G/A |
| 1 | 67614189 | G/A |
| 1 | 67620446 | C/A |
| 1 | 67629340 | G/A |
| 1 | 67675586 | A/T |
| 1 | 67677731 | T/C |
| 1 | 67687076 | C/G |
| 1 | 67722928 | G/C |
| 1 | 67743328 | T/C |
| 1 | 67794446 | T/C |
| 1 | 67795636 | C/G |
| 1 | 57812132 | G/A |
| 1 | 67838933 | A/G |
| 1 | 67845598 | G/T |
| 1 | 67871666 | G/A |
| 1 | 67895258 | A/T |
| 1 | 67923557 | G/A |
| 1 | 67939668 | T/A |
| 1 | 67956613 | C/T |
| 1 | 67994995 | G/A |
| 1 | 68010485 | A/G |
| 1 | 68018833 | G/A |
| 1 | 68069872 | C/T |
| 1 | 68072439 | G/T |
| 1 | 68091151 | T/C |
| 1 | 68117007 | A/G |
| 1 | 68140399 | A/G |
| 1 | 68144280 | A/G |
| 1 | 68173118 | C/A |
| 1 | 68177014 | C/A |
| 1 | 68204292 | C/G |
| 1 | 68247679 | T/C |
| 1 | 68275593 | G/T |
| 1 | 68278501 | A/T |
| 1 | 68280055 | T/A |
| 1 | 68325106 | A/G |
| 1 | 68326441 | C/T |
| 1 | 68359606 | A/T |
| 1 | 68398355 | A/G |
| 1 | 68428755 | C/T |
| 1 | 68446226 | G/A |
| 1 | 68455712 | A/G |
| 1 | 68482518 | A/C |
| 1 | 68513644 | C/A |
| 1 | 68519592 | C/A |
| 1 | 68522508 | A/G |
| 1 | 68546867 | C/T |
| 1 | 68552473 | A/G |
| 1 | 68585749 | G/C |
| 1 | 68596694 | G/A |
| 1 | 68611850 | G/A |
| 1 | 68615733 | T/C |
| 1 | 68657336 | C/T |
| 1 | 68675236 | A/G |
| 1 | 68694405 | C/A |
| 1 | 68702756 | C/T |
| 1 | 68727834 | A/G |
| 1 | 68759640 | T/C |
| 1 | 68777821 | T/C |
| 1 | 68793218 | C/T |
| 1 | 68794634 | A/G |
| 1 | 68837042 | T/A |
| 1 | 68874954 | G/A |
| 1 | 68880129 | A/G |
| 1 | 68926686 | C/T |
| 1 | 68930868 | G/A |
| 1 | 68940284 | T/C |
| 1 | 68963314 | T/C |
| 1 | 68991567 | A/G |
| 1 | 69004901 | A/G |
| 1 | 69033688 | A/G |
| 1 | 69127178 | A/G |
| 1 | 69153071 | G/A |
| 1 | 69193000 | C/G |
| 1 | 69204127 | C/T |
| 1 | 69216938 | T/G |
| 1 | 69255753 | C/T |
| 1 | 69288240 | G/A |
| 1 | 69290380 | G/C |
| 1 | 69306774 | C/T |
| 1 | 69315094 | A/C |
| 1 | 69337846 | C/A |
| 1 | 69341112 | C/G |
| 1 | 69385328 | G/A |
| 1 | 69389480 | A/G |
| 1 | 69399958 | T/C |
| 1 | 69402605 | G/A |
| 1 | 69448865 | T/C |
| 1 | 69466629 | T/G |
| 1 | 69469487 | G/A |
| 1 | 69485214 | C/T |
| 1 | 69512336 | A/C |
| 1 | 69548649 | C/T |
| 1 | 69570270 | A/T |
| 1 | 69576372 | G/A |
| 1 | 69612472 | G/A |
| 1 | 69626379 | T/C |
| 1 | 69645548 | T/C |
| 1 | 69657793 | G/A |
| 1 | 69664263 | G/C |
| 1 | 69668833 | A/G |
| 1 | 69711615 | C/A |
| 1 | 69743451 | G/T |
| 1 | 69776912 | G/C |
| 1 | 69781783 | A/T |
| 1 | 69789773 | G/A |
| 1 | 69810374 | G/C |
| 1 | 69811534 | T/A |
| 1 | 69847461 | G/A |
| 1 | 69848480 | A/T |
| 1 | 69879816 | T/C |
| 1 | 69885121 | T/C |
| 1 | 69900922 | C/A |
| 1 | 69923393 | C/T |
| 1 | 69939441 | T/A |
| 1 | 69949605 | C/T |
| 1 | 69984647 | A/G |
| 1 | 69992871 | G/A |
| 1 | 70015030 | A/G |
| 1 | 70045878 | T/C |
| 1 | 70046940 | T/C |
| 1 | 70072163 | A/G |
| 1 | 70091275 | T/C |
| 1 | 70154623 | G/A |
| 1 | 70187739 | T/C |
| 1 | 70224022 | T/G |
| 1 | 70237131 | T/A |
| 1 | 70257061 | G/T |
| 1 | 70260520 | G/T |
| 1 | 70270629 | T/C |
| 1 | 70293782 | A/C |
| 1 | 70376272 | G/A |
| 1 | 70407344 | G/A |
| 1 | 70414778 | A/T |
| 1 | 70451550 | G/A |
| 1 | 70466416 | A/G |
| 1 | 70474214 | G/A |
| 1 | 70475440 | A/C |
| 1 | 70510373 | C/A |
| 1 | 70512391 | G/A |
| 1 | 70558716 | C/T |
| 1 | 70563281 | T/A |
| 1 | 70606085 | G/A |
| 1 | 70617003 | A/G |
| 1 | 70640508 | G/T |
| 1 | 70650247 | A/G |
| 1 | 70666374 | G/A |
| 1 | 70686165 | C/T |
| 1 | 70715848 | C/T |
| 1 | 70745626 | C/T |
| 1 | 70780339 | A/G |
| 1 | 70855546 | C/A |
| 1 | 70860051 | T/C |
| 1 | 70878609 | C/T |
| 1 | 70891769 | A/G |
| 1 | 70921330 | G/C |
| 1 | 70926302 | A/G |
| 1 | 70950053 | A/G |
| 1 | 70973285 | G/A |
| 1 | 70994329 | T/G |
| 1 | 70996853 | C/G |
| 1 | 71016231 | G/A |
| 1 | 71036430 | G/A |
| 1 | 71042782 | T/C |
| 1 | 71098775 | C/A |
| 1 | 71108147 | A/T |
| 1 | 71131218 | T/C |
| 1 | 71151187 | C/T |
| 1 | 71179868 | T/G |
| 1 | 71196434 | G/A |
| 1 | 71230433 | C/T |
| 1 | 71263861 | G/T |
| 1 | 71296357 | G/A |
| 1 | 71312403 | A/G |
| 1 | 71359766 | A/C |
| 1 | 71373450 | G/A |
| 1 | 71377381 | A/G |
| 1 | 71413939 | T/C |
| 1 | 71456879 | C/G |
| 1 | 71472790 | C/G |
| 1 | 71497727 | C/T |
| 1 | 71525597 | A/T |
| 1 | 71555463 | G/A |
| 1 | 71578192 | G/T |
| 1 | 71580670 | A/G |
| 1 | 71590215 | G/A |
| 1 | 71621188 | A/G |
| 1 | 71631212 | C/T |
| 1 | 71647118 | G/A |
| 1 | 71656915 | C/T |
| 1 | 71690691 | G/A |
| 1 | 71704811 | C/G |
| 1 | 71732076 | G/A |
| 1 | 71744897 | C/T |
| 1 | 71762113 | G/A |
| 1 | 71769606 | A/G |
| 1 | 71795824 | C/T |
| 1 | 71832339 | C/A |
| 1 | 71842819 | G/A |
| 1 | 71866003 | A/C |
| 1 | 71911528 | G/C |
| 1 | 71915248 | T/C |
| 1 | 71964382 | C/A |
| 1 | 71966718 | A/C |
| 1 | 71977247 | C/G |
| 1 | 71988056 | C/T |
| 1 | 72002051 | A/G |
| 1 | 72021855 | T/G |
| 1 | 72038427 | T/C |
| 1 | 72054844 | A/T |
| 1 | 72060531 | A/T |
| 1 | 72094012 | G/A |
| 1 | 72109787 | A/T |
| 1 | 72126167 | A/G |
| 1 | 72161174 | G/A |
| 1 | 72181954 | A/T |
| 1 | 72201933 | C/A |
| 1 | 72234401 | G/T |
| 1 | 72240226 | C/T |
| 1 | 72288486 | G/C |
| 1 | 72293614 | T/C |
| 1 | 72317291 | C/T |
| 1 | 72334959 | C/T |
| 1 | 72336565 | G/A |
| 1 | 72372501 | T/A |
| 1 | 72391356 | C/T |
| 1 | 72418085 | A/G |
| 1 | 72435886 | G/T |
| 1 | 72454021 | A/G |
| 1 | 72466019 | T/C |
| 1 | 72481682 | C/T |
| 1 | 72505507 | T/C |
| 1 | 72512128 | T/C |
| 1 | 72515569 | G/C |
| 1 | 72544126 | C/T |
| 1 | 72561447 | G/C |
| 1 | 72574350 | C/A |
| 1 | 72583317 | C/A |
| 1 | 72609492 | A/G |
| 1 | 72634767 | G/C |
| 1 | 72670069 | T/G |
| 1 | 72682072 | T/C |
| 1 | 72699220 | G/A |
| 1 | 72703837 | A/G |
| 1 | 72723437 | G/A |
| 1 | 72738470 | G/C |
| 1 | 72750786 | G/A |
| 1 | 72755326 | A/T |
| 2 | 37022 | G/A |
| 2 | 70534 | G/A |
| 2 | 91817 | G/A |
| 2 | 150267 | T/G |
| 2 | 160594 | C/T |
| 2 | 202488 | C/T |
| 2 | 299956 | T/A |
| 2 | 314785 | G/A |
| 2 | 325718 | T/A |
| 2 | 348400 | T/C |
| 2 | 358447 | C/A |
| 2 | 403985 | G/C |
| 2 | 428507 | G/T |
| 2 | 464600 | C/T |
| 2 | 544727 | A/G |
| 2 | 545964 | C/A |
| 2 | 594163 | G/A |
| 2 | 611288 | G/T |
| 2 | 612405 | G/A |
| 2 | 661439 | G/A |
| 2 | 720514 | T/C |
| 2 | 761926 | G/A |
| 2 | 782377 | C/T |
| 2 | 817952 | A/T |
| 2 | 824030 | A/G |
| 2 | 850540 | C/T |
| 2 | 899258 | A/G |
| 2 | 904907 | C/T |
| 2 | 914512 | G/C |
| 2 | 958462 | T/C |
| 2 | 958463 | C/T |
| 2 | 958464 | C/T |
| 2 | 958465 | C/T |
| 2 | 958466 | G/A |
| 2 | 958467 | G/T |
| 2 | 1113611 | C/G |
| 2 | 1142286 | C/T |
| 2 | 1190962 | C/G |
| 2 | 1261839 | A/C |
| 2 | 1342172 | A/C |
| 2 | 1371974 | G/A |
| 2 | 1421628 | A/G |
| 2 | 1553687 | C/A |
| 2 | 1561913 | G/T |
| 2 | 1597754 | A/G |
| 2 | 1609638 | G/A |
| 2 | 1647108 | A/G |
| 2 | 1661330 | A/G |
| 2 | 1697266 | G/A |
| 2 | 1717401 | C/T |
| 2 | 1729632 | A/C |
| 2 | 1746152 | G/A |
| 2 | 1759772 | A/C |
| 2 | 1797790 | A/G |
| 2 | 1801979 | T/C |
| 2 | 1836315 | C/T |
| 2 | 1848756 | C/A |
| 2 | 1864779 | G/A |
| 2 | 1899672 | G/A |
| 2 | 1914988 | G/T |
| 2 | 1920012 | A/G |
| 2 | 1941453 | T/A |
| 2 | 1950348 | C/T |
| 2 | 1962723 | G/T |
| 2 | 1992711 | C/T |
| 2 | 2010930 | A/C |
| 2 | 2030592 | T/A |
| 2 | 2061777 | T/C |
| 2 | 2068201 | A/G |
| 2 | 2093902 | G/C |
| 2 | 2127655 | T/C |
| 2 | 2256168 | G/A |
| 2 | 2298532 | G/A |
| 2 | 2334257 | T/C |
| 2 | 2337253 | C/T |
| 2 | 2369392 | G/A |
| 2 | 2379489 | G/T |
| 2 | 2404524 | G/A |
| 2 | 2459842 | C/G |
| 2 | 2487059 | C/G |
| 2 | 2518713 | T/G |
| 2 | 2520662 | A/C |
| 2 | 2547724 | C/T |
| 2 | 2584659 | T/A |
| 2 | 2609741 | C/G |
| 2 | 2617803 | C/A |
| 2 | 2618911 | A/G |
| 2 | 2641475 | A/T |
| 2 | 2644069 | C/T |
| 2 | 2670646 | C/T |
| 2 | 2678195 | A/G |
| 2 | 2700014 | T/G |
| 2 | 2728138 | G/T |
| 2 | 2731661 | C/A |
| 2 | 2751580 | A/G |
| 2 | 2765223 | G/A |
| 2 | 2798798 | T/C |
| 2 | 2804233 | C/A |
| 2 | 2834046 | G/T |
| 2 | 2947536 | A/G |
| 2 | 2996520 | G/C |
| 2 | 3011018 | C/T |
| 2 | 3100812 | G/C |
| 2 | 3169466 | T/A |
| 2 | 3208226 | A/G |
| 2 | 3245104 | G/A |
| 2 | 3286176 | G/A |
| 2 | 3295039 | C/T |
| 2 | 3319863 | C/A |
| 2 | 3353078 | A/G |
| 2 | 3358759 | T/A |
| 2 | 3399142 | G/A |
| 2 | 3449047 | T/C |
| 2 | 3460710 | A/G |
| 2 | 3465403 | G/A |
| 2 | 3487503 | C/T |
| 2 | 3512390 | A/T |
| 2 | 3537146 | T/G |
| 2 | 3548166 | A/G |
| 2 | 3580304 | T/A |
| 2 | 3588002 | T/C |
| 2 | 3601550 | C/G |
| 2 | 3604454 | A/G |
| 2 | 3634545 | T/C |
| 2 | 3687456 | C/T |
| 2 | 3692398 | G/A |
| 2 | 3694038 | C/T |
| 2 | 3742049 | T/G |
| 2 | 3755511 | T/C |
| 2 | 3765035 | T/C |
| 2 | 3780422 | T/C |
| 2 | 3796025 | A/T |
| 2 | 3811061 | T/C |
| 2 | 3850408 | A/T |
| 2 | 3867058 | G/C |
| 2 | 3878096 | T/C |
| 2 | 3905043 | T/A |
| 2 | 3930288 | G/A |
| 2 | 3952721 | T/A |
| 2 | 3962871 | C/T |
| 2 | 3989353 | A/G |
| 2 | 3992642 | C/G |
| 2 | 4024777 | C/T |
| 2 | 4032242 | T/C |
| 2 | 4076695 | G/A |
| 2 | 4079442 | T/C |
| 2 | 4108246 | C/A |
| 2 | 4111251 | G/A |
| 2 | 4165407 | G/A |
| 2 | 4167348 | G/A |
| 2 | 4175661 | C/T |
| 2 | 4216981 | C/T |
| 2 | 4239914 | G/C |
| 2 | 4266165 | C/A |
| 2 | 4287780 | G/A |
| 2 | 4305725 | C/T |
| 2 | 4309577 | A/G |
| 2 | 4322029 | C/T |
| 2 | 4348643 | C/T |
| 2 | 4370876 | T/A |
| 2 | 4373219 | C/T |
| 2 | 4384672 | C/T |
| 2 | 4415501 | C/T |
| 2 | 4445809 | T/C |
| 2 | 4455217 | A/T |
| 2 | 4473341 | T/C |
| 2 | 4495156 | G/A |
| 2 | 4502871 | A/G |
| 2 | 4512756 | C/T |
| 2 | 4534169 | T/C |
| 2 | 4560096 | G/C |
| 2 | 4563356 | T/G |
| 2 | 4590081 | G/T |
| 2 | 4620889 | C/T |
| 2 | 4628680 | C/T |
| 2 | 4666050 | T/C |
| 2 | 4671335 | T/A |
| 2 | 4687571 | C/G |
| 2 | 4704864 | G/T |
| 2 | 4732845 | G/A |
| 2 | 4742334 | C/T |
| 2 | 4760544 | G/A |
| 2 | 4782331 | T/C |
| 2 | 4789447 | G/A |
| 2 | 4801900 | A/G |
| 2 | 4828968 | A/T |
| 2 | 4834732 | T/C |
| 2 | 4851361 | T/A |
| 2 | 4890677 | C/A |
| 2 | 4924226 | G/C |
| 2 | 4935434 | C/G |
| 2 | 4976085 | C/T |
| 2 | 4981762 | A/G |
| 2 | 5017579 | G/C |
| 2 | 5043389 | A/C |
| 2 | 5045747 | T/A |
| 2 | 5092915 | A/C |
| 2 | 5126618 | G/T |
| 2 | 5130022 | C/G |
| 2 | 5160195 | T/G |
| 2 | 5166236 | G/A |
| 2 | 5217741 | T/A |
| 2 | 5230909 | T/G |
| 2 | 5233749 | T/C |
| 2 | 5254803 | C/A |
| 2 | 5268032 | G/C |
| 2 | 5282609 | C/T |
| 2 | 5317294 | A/G |
| 2 | 5341182 | G/A |
| 2 | 5370692 | G/A |
| 2 | 5401664 | G/A |
| 2 | 5404812 | T/C |
| 2 | 5438523 | C/T |
| 2 | 5460427 | C/G |
| 2 | 5488027 | T/C |
| 2 | 5495070 | T/C |
| 2 | 5539049 | C/T |
| 2 | 5550961 | C/G |
| 2 | 5553384 | T/C |
| 2 | 5609962 | G/A |
| 2 | 5616046 | T/C |
| 2 | 5639297 | C/T |
| 2 | 5641586 | T/C |
| 2 | 5652813 | C/T |
| 2 | 5673280 | C/T |
| 2 | 5767063 | G/C |
| 2 | 5806000 | T/G |
| 2 | 5875317 | G/A |
| 2 | 5882617 | G/A |
| 2 | 5911179 | G/A |
| 2 | 5938563 | A/G |
| 2 | 5961577 | C/T |
| 2 | 5987416 | T/A |
| 2 | 6022110 | A/G |
| 2 | 6054734 | A/G |
| 2 | 6081584 | A/G |
| 2 | 6121064 | G/T |
| 2 | 6213613 | T/C |
| 2 | 6236213 | T/A |
| 2 | 6249722 | A/G |
| 2 | 6280996 | C/T |
| 2 | 6289540 | T/C |
| 2 | 6300488 | A/G |
| 2 | 6312255 | T/C |
| 2 | 6312256 | A/G |
| 2 | 6312257 | A/G |
| 2 | 6312258 | A/G |
| 2 | 6365926 | C/T |
| 2 | 6377000 | G/A |
| 2 | 6417505 | A/T |
| 2 | 6418911 | C/T |
| 2 | 6427331 | G/A |
| 2 | 6452434 | G/A |
| 2 | 6511917 | A/G |
| 2 | 6541001 | G/A |
| 2 | 6556487 | T/G |
| 2 | 6570867 | A/G |
| 2 | 6585024 | T/G |
| 2 | 6600862 | T/A |
| 2 | 6632056 | G/C |
| 2 | 6646003 | C/A |
| 2 | 6661002 | C/T |
| 2 | 6672201 | G/A |
| 2 | 6692981 | C/T |
| 2 | 6738725 | G/A |
| 2 | 6779193 | C/T |
| 2 | 6781160 | A/G |
| 2 | 6805611 | T/G |
| 2 | 6810101 | G/C |
| 2 | 6840063 | A/G |
| 2 | 6854732 | A/T |
| 2 | 6870345 | G/C |
| 2 | 6934427 | C/G |
| 2 | 6949203 | G/A |
| 2 | 6961010 | T/C |
| 2 | 6974988 | T/A |
| 2 | 6990140 | G/T |
| 2 | 7024801 | C/G |
| 2 | 7043876 | A/T |
| 2 | 7096721 | A/G |
| 2 | 7108020 | C/T |
| 2 | 7117083 | A/G |
| 2 | 7122999 | C/T |
| 2 | 7148250 | G/A |
| 2 | 7153285 | C/T |
| 2 | 7179365 | A/C |
| 2 | 7191399 | A/G |
| 2 | 7201891 | C/T |
| 2 | 7211414 | T/C |
| 2 | 7230717 | G/A |
| 2 | 7274604 | T/G |
| 2 | 7280839 | A/C |
| 2 | 7290024 | A/G |
| 2 | 7302638 | A/G |
| 2 | 7326769 | C/A |
| 2 | 7404880 | G/T |
| 2 | 7417863 | G/T |
| 2 | 7439721 | C/T |
| 2 | 7462738 | G/C |
| 2 | 7471184 | G/C |
| 2 | 7488955 | A/G |
| 2 | 7501621 | T/A |
| 2 | 7554381 | C/A |
| 2 | 7556265 | A/G |
| 2 | 7563443 | T/A |
| 2 | 7571401 | A/T |
| 2 | 7603484 | G/A |
| 2 | 7607446 | G/A |
| 2 | 7632675 | T/G |
| 2 | 7633907 | C/T |
| 2 | 7650181 | G/A |
| 2 | 7699802 | A/T |
| 2 | 7731921 | A/G |
| 2 | 7741583 | C/T |
| 2 | 7745100 | G/C |
| 2 | 7782628 | T/C |
| 2 | 7785274 | T/C |
| 2 | 7831213 | T/C |
| 2 | 7839702 | C/T |
| 2 | 7886290 | C/T |
| 2 | 7909570 | T/C |
| 2 | 7966830 | C/T |
| 2 | 7978855 | G/A |
| 2 | 7992442 | C/G |
| 2 | 8011061 | T/A |
| 2 | 8044123 | A/G |
| 2 | 8071128 | A/G |
| 2 | 8096224 | C/A |
| 2 | 8105404 | T/A |
| 2 | 8131746 | G/C |
| 2 | 8156939 | C/G |
| 2 | 8163130 | A/G |
| 2 | 8170250 | G/A |
| 2 | 8212455 | G/A |
| 2 | 8218814 | A/G |
| 2 | 8222426 | T/A |
| 2 | 8246056 | T/C |
| 2 | 8251614 | C/T |
| 2 | 8256190 | G/C |
| 2 | 8283613 | G/T |
| 2 | 8290040 | A/T |
| 2 | 8317506 | C/T |
| 2 | 8340543 | A/G |
| 2 | 8370571 | C/T |
| 2 | 8398722 | C/A |
| 2 | 8411721 | T/A |
| 2 | 8423969 | G/A |
| 2 | 8437851 | C/T |
| 2 | 8466186 | T/C |
| 2 | 8472609 | G/C |
| 2 | 8498898 | C/T |
| 2 | 8508104 | G/C |
| 2 | 8523014 | C/G |
| 2 | 8553391 | A/G |
| 2 | 8601347 | T/C |
| 2 | 8616293 | T/G |
| 2 | 8663548 | A/G |
| 2 | 8672046 | A/T |
| 2 | 8673485 | A/G |
| 2 | 8720901 | T/G |
| 2 | 8737834 | C/T |
| 2 | 8765209 | T/A |
| 2 | 8773354 | G/A |
| 2 | 8803979 | T/C |
| 2 | 8827199 | A/C |
| 2 | 8844196 | G/A |
| 2 | 8850218 | A/G |
| 2 | 8883171 | C/G |
| 2 | 8912666 | G/T |
| 2 | 8944463 | T/A |
| 2 | 8975839 | G/A |
| 2 | 8986017 | C/T |
| 2 | 9034680 | T/C |
| 2 | 9058813 | T/C |
| 2 | 9060743 | T/C |
| 2 | 9093179 | A/C |
| 2 | 9098758 | G/C |
| 2 | 9123112 | T/C |
| 2 | 9161115 | T/C |
| 2 | 9166402 | T/G |
| 2 | 9194903 | G/T |
| 2 | 9196431 | A/C |
| 2 | 9233191 | G/A |
| 2 | 9286475 | A/C |
| 2 | 9292521 | G/A |
| 2 | 9305245 | G/A |
| 2 | 9339774 | G/A |
| 2 | 9341474 | G/A |
| 2 | 9371365 | T/G |
| 2 | 9383982 | G/A |
| 2 | 9393728 | G/C |
| 2 | 9413163 | G/T |
| 2 | 9423674 | C/T |
| 2 | 9443869 | G/C |
| 2 | 9465040 | T/C |
| 2 | 9481379 | T/G |
| 2 | 9485105 | T/G |
| 2 | 9522141 | C/A |
| 2 | 9540708 | A/T |
| 2 | 9581928 | C/T |
| 2 | 9618370 | A/G |
| 2 | 9654411 | A/G |
| 2 | 9680884 | G/A |
| 2 | 9689847 | A/G |
| 2 | 9693326 | T/G |
| 2 | 9711563 | T/C |
| 2 | 9720249 | T/C |
| 2 | 9739844 | C/T |
| 2 | 9752137 | T/C |
| 2 | 9772528 | A/G |
| 2 | 9789149 | G/A |
| 2 | 9792954 | A/G |
| 2 | 9831837 | G/A |
| 2 | 9832953 | C/G |
| 2 | 9841475 | G/C |
| 2 | 9868399 | G/A |
| 2 | 9900868 | C/T |
| 2 | 9903169 | C/T |
| 2 | 9940567 | C/G |
| 2 | 9968026 | A/T |
| 2 | 9992291 | T/C |
| 2 | 10016444 | T/C |
| 2 | 10024612 | G/A |
| 2 | 10042170 | G/T |
| 2 | 10055697 | C/G |
| 2 | 10057628 | T/A |
| 2 | 10088304 | G/A |
| 2 | 10115162 | G/T |
| 2 | 10118860 | C/A |
| 2 | 10144370 | T/C |
| 2 | 10149031 | C/A |
| 2 | 10172643 | C/T |
| 2 | 10186890 | A/G |
| 2 | 10201016 | G/T |
| 2 | 10211335 | G/A |
| 2 | 10258497 | C/G |
| 2 | 10279226 | T/C |
| 2 | 10282317 | G/A |
| 2 | 10312435 | A/G |
| 2 | 10345268 | T/C |
| 2 | 10378983 | G/A |
| 2 | 10407105 | A/C |
| 2 | 10435374 | A/T |
| 2 | 10438219 | T/G |
| 2 | 10444611 | C/G |
| 2 | 10479709 | A/G |
| 2 | 10492401 | C/T |
| 2 | 10508467 | T/C |
| 2 | 10523417 | A/T |
| 2 | 10537511 | G/A |
| 2 | 10548693 | G/C |
| 2 | 10569337 | T/A |
| 2 | 10593475 | C/T |
| 2 | 10620368 | G/C |
| 2 | 10640046 | T/G |
| 2 | 10657972 | T/C |
| 2 | 10699735 | A/G |
| 2 | 10703601 | C/T |
| 2 | 10721762 | C/G |
| 2 | 10743703 | C/G |
| 2 | 10744987 | A/T |
| 2 | 10781571 | A/G |
| 2 | 10787780 | T/C |
| 2 | 10808497 | C/T |
| 2 | 10816918 | T/C |
| 2 | 10832620 | T/A |
| 2 | 10877921 | C/T |
| 2 | 10910619 | G/A |
| 2 | 10933500 | G/A |
| 2 | 10957888 | T/C |
| 2 | 10985995 | C/T |
| 2 | 11001784 | G/T |
| 2 | 11018809 | G/A |
| 2 | 11054882 | C/T |
| 2 | 11063278 | C/T |
| 2 | 11070945 | T/C |
| 2 | 11079701 | C/A |
| 2 | 11101749 | A/G |
| 2 | 11105808 | C/T |
| 2 | 11132089 | A/G |
| 2 | 11181745 | G/A |
| 2 | 11198686 | C/T |
| 2 | 11233905 | C/T |
| 2 | 11288937 | G/A |
| 2 | 11325893 | C/G |
| 2 | 11335607 | G/A |
| 2 | 11369161 | G/A |
| 2 | 11370878 | A/G |
| 2 | 11380226 | G/A |
| 2 | 11404067 | C/T |
| 2 | 11421293 | C/T |
| 2 | 11446492 | C/T |
| 2 | 11486623 | C/G |
| 2 | 11503006 | G/A |
| 2 | 11516778 | C/A |
| 2 | 11542671 | C/T |
| 2 | 11562146 | C/A |
| 2 | 11583690 | T/G |
| 2 | 11605825 | C/T |
| 2 | 11612139 | T/A |
| 2 | 11687303 | G/C |
| 2 | 11724732 | A/T |
| 2 | 11744418 | T/C |
| 2 | 11757791 | T/C |
| 2 | 11769469 | G/T |
| 2 | 11783597 | G/A |
| 2 | 11801122 | T/C |
| 2 | 11835390 | T/C |
| 2 | 11857133 | C/A |
| 2 | 11866537 | G/A |
| 2 | 11880374 | A/G |
| 2 | 11903022 | A/T |
| 2 | 11913899 | T/C |
| 2 | 11941982 | A/T |
| 2 | 11982699 | C/A |
| 2 | 11987331 | A/G |
| 2 | 12008290 | A/G |
| 2 | 12011141 | T/G |
| 2 | 12037664 | T/C |
| 2 | 12040111 | A/C |
| 2 | 12069847 | C/T |
| 2 | 12134110 | T/G |
| 2 | 12144084 | C/T |
| 2 | 12151166 | C/T |
| 2 | 12174161 | G/A |
| 2 | 12180259 | C/T |
| 2 | 12201077 | A/C |
| 2 | 12239555 | T/A |
| 2 | 12263233 | T/G |
| 2 | 12286488 | T/C |
| 2 | 12296043 | G/A |
| 2 | 12304248 | C/T |
| 2 | 12315509 | A/G |
| 2 | 12349098 | C/T |
| 2 | 12386364 | T/A |
| 2 | 12412106 | C/T |
| 2 | 12438050 | G/T |
| 2 | 12459767 | A/G |
| 2 | 12459768 | G/A |
| 2 | 12459769 | G/A |
| 2 | 12459770 | G/A |
| 2 | 12543412 | G/A |
| 2 | 12551299 | T/G |
| 2 | 12551300 | G/A |
| 2 | 12551301 | G/A |
| 2 | 12627503 | G/A |
| 2 | 12647099 | A/G |
| 2 | 12658668 | C/T |
| 2 | 12676569 | A/G |
| 2 | 12684237 | T/G |
| 2 | 12703075 | T/A |
| 2 | 12719450 | G/A |
| 2 | 12740154 | A/G |
| 2 | 12761118 | A/G |
| 2 | 12785329 | C/T |
| 2 | 12790601 | C/T |
| 2 | 12834026 | C/G |
| 2 | 12844892 | G/C |
| 2 | 12879623 | G/T |
| 2 | 12906236 | G/C |
| 2 | 12917013 | G/A |
| 2 | 12948600 | A/G |
| 2 | 12962995 | T/G |
| 2 | 12962996 | A/T |
| 2 | 12962997 | A/T |
| 2 | 12962998 | A/T |
| 2 | 13041365 | A/T |
| 2 | 13068276 | C/T |
| 2 | 13085817 | G/A |
| 2 | 13118709 | G/A |
| 2 | 13123775 | G/A |
| 2 | 13153092 | C/T |
| 2 | 13185188 | A/G |
| 2 | 13237945 | T/C |
| 2 | 13239967 | T/A |
| 2 | 13271578 | C/A |
| 2 | 13288336 | C/T |
| 2 | 13310789 | T/C |
| 2 | 13345854 | A/C |
| 2 | 13351356 | T/C |
| 2 | 13392082 | C/T |
| 2 | 13407035 | A/G |
| 2 | 13412894 | G/A |
| 2 | 13414500 | A/G |
| 2 | 13445319 | G/T |
| 2 | 13452123 | A/C |
| 2 | 13499446 | C/G |
| 2 | 13503811 | C/T |
| 2 | 13546053 | G/A |
| 2 | 13562882 | C/T |
| 2 | 13563924 | C/T |
| 2 | 13594681 | A/G |
| 2 | 13631259 | C/T |
| 2 | 13650144 | T/C |
| 2 | 13681226 | G/A |
| 2 | 13735788 | G/A |
| 2 | 13755050 | A/T |
| 2 | 13798941 | T/C |
| 2 | 13803371 | C/G |
| 2 | 13806110 | G/A |
| 2 | 13888006 | G/A |
| 2 | 13910393 | A/G |
| 2 | 13931814 | C/T |
| 2 | 13935782 | G/A |
| 2 | 13956160 | G/A |
| 2 | 13996006 | T/C |
| 2 | 14012068 | T/C |
| 2 | 14085298 | T/C |
| 2 | 14090180 | C/T |
| 2 | 14114417 | C/T |
| 2 | 14142846 | G/A |
| 2 | 14175467 | A/G |
| 2 | 14207806 | G/A |
| 2 | 14213043 | C/G |
| 2 | 14226666 | C/G |
| 2 | 14238238 | G/A |
| 2 | 14263862 | A/G |
| 2 | 14283022 | A/C |
| 2 | 14305368 | C/G |
| 2 | 14316050 | G/C |
| 2 | 14320892 | G/A |
| 2 | 14361132 | A/G |
| 2 | 14366526 | C/T |
| 2 | 14370237 | T/C |
| 2 | 14380766 | C/T |
| 2 | 14401894 | A/G |
| 2 | 14434671 | A/T |
| 2 | 14450128 | T/C |
| 2 | 14466558 | G/A |
| 2 | 14495870 | G/C |
| 2 | 14515290 | G/T |
| 2 | 14522380 | T/C |
| 2 | 14530127 | A/G |
| 2 | 14555876 | A/C |
| 2 | 14584944 | G/A |
| 2 | 14586923 | C/T |
| 2 | 14627554 | G/A |
| 2 | 14631965 | T/C |
| 2 | 14665711 | T/C |
| 2 | 14693019 | A/G |
| 2 | 14693020 | G/A |
| 2 | 14713880 | C/T |
| 2 | 14736285 | A/G |
| 2 | 14741968 | G/T |
| 2 | 14774651 | G/A |
| 2 | 14793722 | G/A |
| 2 | 14799454 | A/G |
| 2 | 14826193 | C/T |
| 2 | 14833931 | A/G |
| 2 | 14876548 | C/T |
| 2 | 14879804 | G/A |
| 2 | 14879805 | T/C |
| 2 | 14879806 | T/C |
| 2 | 14879807 | T/C |
| 2 | 14951819 | G/A |
| 2 | 14997714 | A/C |
| 2 | 15036638 | C/T |
| 2 | 15052162 | A/G |
| 2 | 15066847 | G/A |
| 2 | 15084040 | C/T |
| 2 | 15096469 | G/A |
| 2 | 15126742 | A/T |
| 2 | 15133451 | A/G |
| 2 | 15150631 | G/A |
| 2 | 15161269 | C/T |
| 2 | 15235595 | C/T |
| 2 | 15240653 | G/A |
| 2 | 15264057 | C/T |
| 2 | 15289520 | G/A |
| 2 | 15293847 | T/C |
| 2 | 15315713 | G/A |
| 2 | 15338161 | G/A |
| 2 | 15394154 | C/T |
| 2 | 15415528 | G/A |
| 2 | 15443332 | A/G |
| 2 | 15500955 | G/A |
| 2 | 15671628 | A/C |
| 2 | 15685726 | T/A |
| 2 | 15690355 | T/C |
| 2 | 15723478 | G/T |
| 2 | 15739415 | G/A |
| 2 | 15754934 | C/A |
| 2 | 15768640 | G/A |
| 2 | 15791877 | A/G |
| 2 | 15817131 | T/G |
| 2 | 15822486 | C/T |
| 2 | 15841865 | T/C |
| 2 | 15868702 | T/C |
| 2 | 15871877 | G/A |
| 2 | 15879641 | T/A |
| 2 | 15905229 | T/A |
| 2 | 15931614 | G/C |
| 2 | 15935485 | A/T |
| 2 | 15990953 | T/C |
| 2 | 15997026 | C/T |
| 2 | 16040655 | C/A |
| 2 | 16050765 | T/C |
| 2 | 16067119 | G/C |
| 2 | 16087675 | C/T |
| 2 | 16113999 | T/G |
| 2 | 16134297 | C/A |
| 2 | 16152749 | A/G |
| 2 | 16166234 | T/C |
| 2 | 16195493 | G/A |
| 2 | 16209635 | C/G |
| 2 | 16258307 | T/C |
| 2 | 16263508 | C/T |
| 2 | 16283489 | A/C |
| 2 | 16291999 | C/T |
| 2 | 16303945 | T/A |
| 2 | 16323562 | G/A |
| 2 | 16364307 | T/C |
| 2 | 16378524 | T/A |
| 2 | 16378525 | C/T |
| 2 | 16378526 | C/T |
| 2 | 16378527 | C/T |
| 2 | 16378528 | G/A |
| 2 | 16378529 | G/A |
| 2 | 16378530 | G/A |
| 2 | 16378531 | C/T |
| 2 | 16378532 | C/T |
| 2 | 16378533 | C/T |
| 2 | 16473201 | C/T |
| 2 | 16474277 | C/T |
| 2 | 16527416 | G/A |
| 2 | 16532881 | A/G |
| 2 | 16569059 | C/T |
| 2 | 16589463 | G/T |
| 2 | 16599267 | C/T |
| 2 | 16631687 | G/T |
| 2 | 16641931 | C/T |
| 2 | 16650945 | T/C |
| 2 | 16706587 | G/A |
| 2 | 16736336 | C/T |
| 2 | 16740265 | A/G |
| 2 | 16744372 | C/T |
| 2 | 16795484 | A/G |
| 2 | 16810853 | A/G |
| 2 | 16816828 | A/G |
| 2 | 16830325 | G/A |
| 2 | 16872122 | G/A |
| 2 | 16878594 | T/C |
| 2 | 16897420 | G/T |
| 2 | 16932105 | C/A |
| 2 | 16965196 | T/C |
| 2 | 16990323 | A/C |
| 2 | 17009426 | G/A |
| 2 | 17034113 | G/A |
| 2 | 17035756 | G/A |
| 2 | 17051543 | T/C |
| 2 | 17055469 | G/T |
| 2 | 17081949 | G/A |
| 2 | 17100497 | A/G |
| 2 | 17115948 | T/G |
| 2 | 17157580 | T/G |
| 2 | 17174800 | T/C |
| 2 | 17180531 | C/T |
| 2 | 17203158 | A/G |
| 2 | 17229331 | G/A |
| 2 | 17246909 | G/A |
| 2 | 17269476 | G/A |
| 2 | 17277891 | G/A |
| 2 | 17299743 | C/T |
| 2 | 17314652 | T/C |
| 2 | 17342213 | C/T |
| 2 | 17357414 | A/G |
| 2 | 17373602 | G/A |
| 2 | 17375722 | G/T |
| 2 | 17422865 | G/C |
| 2 | 17428573 | G/A |
| 2 | 17433019 | G/T |
| 2 | 17457902 | G/T |
| 2 | 17466516 | C/G |
| 2 | 17468780 | C/T |
| 2 | 17510075 | G/A |
| 2 | 17531479 | C/T |
| 2 | 17542222 | T/C |
| 2 | 17551795 | A/T |
| 2 | 17580723 | T/C |
| 2 | 17582371 | A/G |
| 2 | 17611637 | C/T |
| 2 | 17620302 | C/A |
| 2 | 17645152 | C/A |
| 2 | 17659881 | C/T |
| 2 | 17674095 | G/C |
| 2 | 17684441 | G/C |
| 2 | 17706888 | C/G |
| 2 | 17713406 | G/A |
| 2 | 17735032 | A/G |
| 2 | 17750772 | A/T |
| 2 | 17785727 | T/C |
| 2 | 17810419 | G/A |
| 2 | 17818143 | A/T |
| 2 | 17821333 | C/T |
| 2 | 17833494 | C/A |
| 2 | 17874612 | C/A |
| 2 | 17876250 | T/C |
| 2 | 17887552 | G/A |
| 2 | 17910243 | G/A |
| 2 | 17945294 | T/C |
| 2 | 17959470 | C/T |
| 2 | 17973691 | C/T |
| 2 | 17980928 | A/G |
| 2 | 18002628 | T/G |
| 2 | 18028750 | A/G |
| 2 | 18048111 | A/C |
| 2 | 18076178 | C/T |
| 2 | 18099487 | T/A |
| 2 | 18122614 | C/G |
| 2 | 18147534 | C/G |
| 2 | 18178172 | T/C |
| 2 | 18191180 | C/T |
| 2 | 18228846 | C/T |
| 2 | 18261721 | G/A |
| 2 | 18289965 | G/A |
| 2 | 18304804 | T/C |
| 2 | 18323159 | C/A |
| 2 | 18331398 | G/A |
| 2 | 18331399 | T/C |
| 2 | 18331400 | T/C |
| 2 | 18331401 | T/C |
| 2 | 18415762 | A/T |
| 2 | 18418885 | C/A |
| 2 | 18435042 | C/T |
| 2 | 18468124 | G/A |
| 2 | 18500861 | C/A |
| 2 | 18505242 | G/A |
| 2 | 18529673 | G/A |
| 2 | 18539905 | G/A |
| 2 | 18550352 | T/G |
| 2 | 18586426 | A/T |
| 2 | 18604924 | T/C |
| 2 | 18631210 | T/C |
| 2 | 18638014 | G/T |
| 2 | 18668991 | C/T |
| 2 | 18679986 | A/G |
| 2 | 18715045 | C/T |
| 2 | 18716721 | G/A |
| 2 | 18724336 | C/T |
| 2 | 18746390 | C/T |
| 2 | 18761469 | G/A |
| 2 | 18777088 | G/A |
| 2 | 18791964 | C/T |
| 2 | 18793769 | C/T |
| 2 | 18827519 | T/C |
| 2 | 18844012 | A/G |
| 2 | 18860271 | A/G |
| 2 | 18876456 | G/A |
| 2 | 18892180 | G/A |
| 2 | 18903202 | C/T |
| 2 | 18915867 | A/G |
| 2 | 18933523 | G/C |
| 2 | 18953090 | C/T |
| 2 | 18979194 | T/G |
| 2 | 18981217 | T/C |
| 2 | 18999529 | G/A |
| 2 | 19018507 | G/A |
| 2 | 19040552 | T/A |
| 2 | 19053681 | G/C |
| 2 | 19062430 | G/A |
| 2 | 19108019 | G/T |
| 2 | 19122450 | C/T |
| 2 | 19124098 | A/C |
| 2 | 19155235 | G/A |
| 2 | 19165290 | A/G |
| 2 | 19170771 | C/T |
| 2 | 19236856 | C/A |
| 2 | 19273872 | A/G |
| 2 | 19295972 | G/A |
| 2 | 19310185 | G/A |
| 2 | 19321202 | C/T |
| 2 | 19360762 | C/T |
| 2 | 19408301 | C/T |
| 2 | 19429864 | T/C |
| 2 | 19439017 | A/G |
| 2 | 19441281 | A/G |
| 2 | 19489337 | C/A |
| 2 | 19497333 | A/G |
| 2 | 19512732 | T/C |
| 2 | 19520985 | C/T |
| 2 | 19557277 | A/G |
| 2 | 19577723 | A/G |
| 2 | 19582469 | T/G |
| 2 | 19593268 | A/G |
| 2 | 19595922 | A/G |
| 2 | 19647560 | C/T |
| 2 | 19671088 | A/T |
| 2 | 19684252 | G/A |
| 2 | 19720302 | A/T |
| 2 | 19744679 | G/A |
| 2 | 19779740 | C/T |
| 2 | 19797085 | T/G |
| 2 | 19813883 | C/T |
| 2 | 19846787 | G/A |
| 2 | 19882448 | C/G |
| 2 | 19883828 | G/A |
| 2 | 19902682 | C/T |
| 2 | 19931489 | A/T |
| 2 | 19945201 | G/A |
| 2 | 19957514 | A/G |
| 2 | 19986711 | C/T |
| 2 | 20002779 | C/T |
| 2 | 20012392 | C/T |
| 2 | 20023174 | G/A |
| 2 | 20053674 | A/C |
| 2 | 20063576 | C/T |
| 2 | 20075005 | G/A |
| 2 | 20122535 | G/A |
| 2 | 20126576 | T/C |
| 2 | 20145161 | G/A |
| 2 | 20148556 | G/A |
| 2 | 20173966 | A/G |
| 2 | 20200184 | G/A |
| 2 | 20269225 | A/T |
| 2 | 20283789 | C/G |
| 2 | 20289765 | C/T |
| 2 | 20310316 | A/T |
| 2 | 20350497 | C/G |
| 2 | 20376802 | A/C |
| 2 | 20419964 | A/G |
| 2 | 20424200 | T/C |
| 2 | 20450641 | A/G |
| 2 | 20453856 | G/A |
| 2 | 20462601 | T/C |
| 2 | 20494580 | T/C |
| 2 | 20515443 | A/G |
| 2 | 20520700 | A/G |
| 2 | 20557461 | G/A |
| 2 | 20580387 | C/T |
| 2 | 20617874 | G/A |
| 2 | 20629380 | G/A |
| 2 | 20652377 | C/A |
| 2 | 20666027 | A/G |
| 2 | 20694704 | C/A |
| 2 | 20701769 | G/C |
| 2 | 20714653 | A/G |
| 2 | 20731093 | T/G |
| 2 | 20744616 | C/T |
| 2 | 20787267 | A/G |
| 2 | 20788894 | C/T |
| 2 | 20797125 | A/C |
| 2 | 20805964 | A/G |
| 2 | 20835882 | C/T |
| 2 | 20862784 | T/C |
| 2 | 20901818 | C/A |
| 2 | 20910124 | A/T |
| 2 | 20943935 | C/T |
| 2 | 20947203 | G/A |
| 2 | 20981789 | C/T |
| 2 | 21011632 | A/G |
| 2 | 21013397 | T/C |
| 2 | 21031707 | C/T |
| 2 | 21033711 | G/A |
| 2 | 21069994 | A/C |
| 2 | 21095229 | C/A |
| 2 | 21098248 | G/A |
| 2 | 21138117 | G/A |
| 2 | 21152312 | G/A |
| 2 | 21165426 | T/C |
| 2 | 21186427 | A/G |
| 2 | 21204058 | G/T |
| 2 | 21217265 | G/A |
| 2 | 21239101 | C/A |
| 2 | 21253483 | C/T |
| 2 | 21266202 | G/T |
| 2 | 21272173 | C/G |
| 2 | 21282908 | G/T |
| 2 | 21302652 | A/C |
| 2 | 21338962 | A/G |
| 2 | 21360576 | T/C |
| 2 | 21369594 | T/C |
| 2 | 21369595 | A/G |
| 2 | 21369596 | A/G |
| 2 | 21369597 | A/G |
| 2 | 21465990 | G/A |
| 2 | 21505485 | C/G |
| 2 | 21508394 | C/T |
| 2 | 21522673 | G/A |
| 2 | 21547303 | T/A |
| 2 | 21549189 | G/A |
| 2 | 21580600 | C/T |
| 2 | 21608664 | G/A |
| 2 | 21637396 | G/A |
| 2 | 21681489 | A/C |
| 2 | 21687936 | T/C |
| 2 | 21693021 | A/C |
| 2 | 21717560 | G/A |
| 2 | 21728829 | T/A |
| 2 | 21731530 | G/A |
| 2 | 21768817 | A/G |
| 2 | 21808612 | A/C |
| 2 | 21830917 | T/C |
| 2 | 21833958 | C/T |
| 2 | 21854326 | A/G |
| 2 | 21887318 | C/A |
| 2 | 21909976 | T/C |
| 2 | 21930612 | C/T |
| 2 | 21934313 | C/T |
| 2 | 21970464 | G/A |
| 2 | 21973305 | T/C |
| 2 | 21995572 | T/C |
| 2 | 22025029 | G/A |
| 2 | 22049650 | T/A |
| 2 | 22054699 | A/G |
| 2 | 22072570 | G/A |
| 2 | 22096378 | C/T |
| 2 | 22103512 | A/G |
| 2 | 22110965 | T/C |
| 2 | 22136936 | A/C |
| 2 | 22151995 | T/G |
| 2 | 22155056 | G/A |
| 2 | 22181532 | C/A |
| 2 | 22213342 | T/A |
| 2 | 22232074 | T/C |
| 2 | 22245674 | C/A |
| 2 | 22262996 | G/C |
| 2 | 22283768 | T/C |
| 2 | 22314941 | G/C |
| 2 | 22326487 | G/T |
| 2 | 22342422 | T/C |
| 2 | 22356247 | T/A |
| 2 | 22369457 | T/C |
| 2 | 22383438 | A/G |
| 2 | 22427268 | C/T |
| 2 | 22432369 | C/T |
| 2 | 22442873 | T/C |
| 2 | 22465448 | C/T |
| 2 | 22472830 | C/G |
| 2 | 22487201 | C/T |
| 2 | 22519856 | T/G |
| 2 | 22532941 | G/A |
| 2 | 22536207 | G/A |
| 2 | 22574810 | C/T |
| 2 | 22597437 | T/G |
| 2 | 22634114 | C/T |
| 2 | 22651095 | A/G |
| 2 | 22653995 | G/T |
| 2 | 22700477 | C/T |
| 2 | 22715431 | C/A |
| 2 | 22734253 | C/T |
| 2 | 22743641 | T/C |
| 2 | 22793513 | G/A |
| 2 | 22826539 | C/T |
| 2 | 22836817 | C/T |
| 2 | 22838019 | A/T |
| 2 | 22866190 | T/G |
| 2 | 22891514 | G/A |
| 2 | 22960118 | T/C |
| 2 | 22985328 | T/A |
| 2 | 23019029 | G/C |
| 2 | 23037456 | G/A |
| 2 | 23041974 | G/A |
| 2 | 23054835 | C/T |
| 2 | 23087127 | T/C |
| 2 | 23104881 | G/A |
| 2 | 23107854 | T/C |
| 2 | 23173687 | C/T |
| 2 | 23184497 | G/A |
| 2 | 23194572 | C/T |
| 2 | 23230411 | G/A |
| 2 | 23248749 | C/T |
| 2 | 23250883 | G/A |
| 2 | 23283150 | C/T |
| 2 | 23351891 | C/T |
| 2 | 23362879 | G/C |
| 2 | 23370872 | T/C |
| 2 | 23389520 | T/A |
| 2 | 23415031 | T/C |
| 2 | 23430266 | C/T |
| 2 | 23475641 | T/C |
| 2 | 23482021 | C/T |
| 2 | 23508993 | A/G |
| 2 | 23541320 | T/A |
| 2 | 23572041 | G/A |
| 2 | 23594336 | A/G |
| 2 | 23611700 | T/C |
| 2 | 23668757 | A/G |
| 2 | 23683504 | G/A |
| 2 | 23685488 | T/A |
| 2 | 23717232 | C/G |
| 2 | 23732279 | C/A |
| 2 | 23763317 | A/G |
| 2 | 23765368 | G/T |
| 2 | 23796701 | G/T |
| 2 | 23804360 | C/T |
| 2 | 23856046 | T/C |
| 2 | 23862064 | G/A |
| 2 | 23916040 | C/T |
| 2 | 23922287 | G/A |
| 2 | 23941853 | C/T |
| 2 | 23962571 | G/C |
| 2 | 23980329 | G/A |
| 2 | 23991302 | T/C |
| 2 | 24012321 | C/T |
| 2 | 24012322 | G/C |
| 2 | 24012323 | G/C |
| 2 | 24012324 | G/C |
| 2 | 24063892 | G/T |
| 2 | 24090621 | G/A |
| 2 | 24138498 | A/C |
| 2 | 24147155 | C/A |
| 2 | 24150269 | T/A |
| 2 | 24179824 | G/A |
| 2 | 24185344 | G/A |
| 2 | 24209495 | C/T |
| 2 | 24213468 | G/T |
| 2 | 24266390 | G/A |
| 2 | 24301532 | C/A |
| 2 | 24315864 | T/C |
| 2 | 24353885 | T/C |
| 2 | 24359875 | G/T |
| 2 | 24361829 | C/G |
| 2 | 24376898 | C/T |
| 2 | 24395271 | G/A |
| 2 | 24407894 | G/A |
| 2 | 24434225 | G/A |
| 2 | 24453606 | A/T |
| 2 | 24471866 | C/T |
| 2 | 24507599 | G/C |
| 2 | 24509309 | T/G |
| 2 | 24519769 | G/A |
| 2 | 24546013 | C/A |
| 2 | 24562218 | A/G |
| 2 | 24582507 | A/G |
| 2 | 24612352 | T/C |
| 2 | 24628313 | G/A |
| 2 | 24648112 | T/G |
| 2 | 24650557 | C/T |
| 2 | 24668911 | C/G |
| 2 | 24695834 | G/A |
| 2 | 24697513 | C/A |
| 2 | 24731761 | G/A |
| 2 | 24740523 | G/T |
| 2 | 24770011 | G/A |
| 2 | 24780421 | T/C |
| 2 | 24810815 | A/G |
| 2 | 24867258 | G/T |
| 2 | 24877281 | A/G |
| 2 | 24881282 | C/T |
| 2 | 24918514 | T/C |
| 2 | 24919683 | C/T |
| 2 | 24939742 | C/T |
| 2 | 24963706 | G/T |
| 2 | 25004166 | A/C |
| 2 | 25013926 | C/T |
| 2 | 25028535 | G/A |
| 2 | 25037567 | C/T |
| 2 | 25075707 | C/T |
| 2 | 25087526 | C/T |
| 2 | 25106408 | C/T |
| 2 | 25113438 | C/T |
| 2 | 25139139 | G/A |
| 2 | 25144432 | G/T |
| 2 | 25154021 | A/G |
| 2 | 25194941 | A/C |
| 2 | 25197717 | A/G |
| 2 | 25200221 | G/C |
| 2 | 25236801 | G/A |
| 2 | 25252638 | C/T |
| 2 | 25276843 | T/C |
| 2 | 25281572 | C/G |
| 2 | 25315597 | C/G |
| 2 | 25330390 | C/T |
| 2 | 25350662 | C/T |
| 2 | 25380244 | G/A |
| 2 | 25404399 | T/C |
| 2 | 25412497 | C/T |
| 2 | 25436257 | C/G |
| 2 | 25441857 | A/G |
| 2 | 25444344 | C/T |
| 2 | 25476268 | T/C |
| 2 | 25494897 | C/T |
| 2 | 25502979 | A/G |
| 2 | 25611868 | T/C |
| 2 | 25614501 | A/G |
| 2 | 25620356 | T/A |
| 2 | 25651769 | G/A |
| 2 | 25687844 | G/A |
| 2 | 25723597 | G/A |
| 2 | 25725850 | T/C |
| 2 | 25747426 | G/A |
| 2 | 25751174 | G/A |
| 2 | 25776893 | T/G |
| 2 | 25798317 | A/G |
| 2 | 25809355 | G/A |
| 2 | 25826383 | C/T |
| 2 | 25832338 | A/T |
| 2 | 25858895 | C/T |
| 2 | 25864324 | T/C |
| 2 | 25873715 | T/C |
| 2 | 25897745 | G/A |
| 2 | 25905699 | C/T |
| 2 | 25922628 | G/A |
| 2 | 25935519 | C/T |
| 2 | 25951603 | T/C |
| 2 | 26006150 | T/C |
| 2 | 26026979 | C/T |
| 2 | 26029506 | A/G |
| 2 | 26063913 | T/C |
| 2 | 26071568 | C/T |
| 2 | 26073627 | G/A |
| 2 | 26106249 | C/T |
| 2 | 26125181 | A/C |
| 2 | 26153072 | G/A |
| 2 | 26160837 | G/A |
| 2 | 26186687 | A/G |
| 2 | 26205724 | T/G |
| 2 | 26238254 | A/G |
| 2 | 26244211 | G/A |
| 2 | 26253911 | T/C |
| 2 | 26294675 | G/A |
| 2 | 26296623 | A/G |
| 2 | 26316651 | G/A |
| 2 | 26361361 | C/T |
| 2 | 26366747 | G/A |
| 2 | 26366748 | G/A |
| 2 | 26366749 | G/A |
| 2 | 26366750 | G/A |
| 2 | 26426073 | A/T |
| 2 | 26440283 | T/C |
| 2 | 26456697 | C/T |
| 2 | 26470751 | A/G |
| 2 | 26508049 | C/T |
| 2 | 26529148 | G/A |
| 2 | 26544493 | C/A |
| 2 | 26552349 | T/C |
| 2 | 26564101 | G/A |
| 2 | 26587392 | G/T |
| 2 | 26608786 | C/T |
| 2 | 26612242 | C/G |
| 2 | 26615070 | C/T |
| 2 | 26647860 | C/T |
| 2 | 26672131 | T/C |
| 2 | 26690008 | A/G |
| 2 | 26717239 | G/A |
| 2 | 26723166 | A/T |
| 2 | 26747671 | C/T |
| 2 | 26758867 | G/C |
| 2 | 26761207 | C/G |
| 2 | 26788967 | G/A |
| 2 | 26801271 | T/C |
| 2 | 26823170 | G/C |
| 2 | 26852672 | T/C |
| 2 | 26862767 | G/A |
| 2 | 26882565 | T/C |
| 2 | 26889078 | T/C |
| 2 | 26929233 | C/T |
| 2 | 26957230 | C/A |
| 2 | 26973238 | T/C |
| 2 | 26974425 | G/A |
| 2 | 26974426 | G/A |
| 2 | 26974427 | G/A |
| 2 | 26974428 | G/A |
| 2 | 27055020 | G/A |
| 2 | 27068568 | G/A |
| 2 | 27074864 | G/A |
| 2 | 27111390 | C/T |
| 2 | 27121996 | C/A |
| 2 | 27143738 | A/G |
| 2 | 27157726 | A/G |
| 2 | 27208011 | C/T |
| 2 | 27213919 | T/G |
| 2 | 27254157 | T/C |
| 2 | 27259797 | T/G |
| 2 | 27270011 | C/T |
| 2 | 27274423 | C/T |
| 2 | 27301053 | C/T |
| 2 | 27312342 | A/C |
| 2 | 27332898 | T/A |
| 2 | 27335081 | G/A |
| 2 | 27378236 | C/A |
| 2 | 27387087 | C/T |
| 2 | 27417393 | G/A |
| 2 | 27427105 | T/G |
| 2 | 27462791 | A/G |
| 2 | 27479498 | G/T |
| 2 | 27486935 | G/T |
| 2 | 27511088 | C/T |
| 2 | 27530315 | C/T |
| 2 | 27543422 | G/T |
| 2 | 27572165 | A/C |
| 2 | 27575513 | A/C |
| 2 | 27625736 | G/A |
| 2 | 27636245 | G/C |
| 2 | 27686419 | T/C |
| 2 | 27689636 | C/T |
| 2 | 27695485 | C/T |
| 2 | 27700180 | A/G |
| 2 | 27738964 | A/G |
| 2 | 27743348 | A/G |
| 2 | 27750802 | G/A |
| 2 | 27755913 | A/G |
| 2 | 27805193 | T/C |
| 2 | 27806302 | T/G |
| 2 | 27814232 | A/G |
| 2 | 27814233 | A/G |
| 2 | 27814234 | A/G |
| 2 | 27814235 | A/G |
| 2 | 27896196 | A/G |
| 2 | 27919265 | C/T |
| 2 | 27946898 | G/A |
| 2 | 27984680 | T/C |
| 2 | 27994487 | G/T |
| 2 | 27997081 | A/G |
| 2 | 28022833 | T/C |
| 2 | 28028410 | G/A |
| 2 | 28057961 | A/C |
| 2 | 28073231 | T/C |
| 2 | 28093534 | C/T |
| 2 | 28093535 | G/A |
| 2 | 28093536 | G/A |
| 2 | 28093537 | G/A |
| 2 | 28154917 | T/G |
| 2 | 28156420 | T/C |
| 2 | 28175525 | T/C |
| 2 | 28200176 | T/C |
| 2 | 28201665 | T/C |
| 2 | 28251532 | A/G |
| 2 | 28253660 | A/G |
| 2 | 28271839 | T/A |
| 2 | 28300071 | C/T |
| 2 | 28330269 | T/C |
| 2 | 28397270 | A/G |
| 2 | 28398438 | A/G |
| 2 | 28433204 | C/G |
| 2 | 28461158 | C/T |
| 2 | 28469575 | C/G |
| 2 | 28472402 | G/A |
| 2 | 28474395 | A/C |
| 2 | 28500830 | G/A |
| 2 | 28508063 | A/G |
| 2 | 28508064 | A/G |
| 2 | 28508065 | A/G |
| 2 | 28508066 | A/G |
| 2 | 28564480 | T/C |
| 2 | 28569940 | T/C |
| 2 | 28592845 | C/T |
| 2 | 28602678 | C/T |
| 2 | 28624210 | A/C |
| 2 | 28656009 | T/A |
| 2 | 28686255 | T/C |
| 2 | 28718197 | T/G |
| 2 | 28720235 | G/A |
| 2 | 28762278 | G/A |
| 2 | 28776996 | T/A |
| 2 | 28788850 | G/A |
| 2 | 28801078 | G/A |
| 2 | 28812680 | G/C |
| 2 | 28857033 | C/T |
| 2 | 28860481 | A/C |
| 2 | 28867783 | T/A |
| 2 | 28867784 | A/G |
| 2 | 28867785 | A/G |
| 2 | 28867786 | A/G |
| 2 | 28963727 | T/C |
| 2 | 28969953 | G/A |
| 2 | 28983050 | T/C |
| 2 | 28992288 | C/T |
| 2 | 29038163 | T/C |
| 2 | 29050158 | C/T |
| 2 | 29064477 | T/C |
| 2 | 29087096 | G/A |
| 2 | 29102247 | C/T |
| 2 | 29120314 | C/G |
| 2 | 29135021 | T/C |
| 2 | 29142425 | A/G |
| 2 | 29179695 | G/A |
| 2 | 29182738 | T/C |
| 2 | 29194763 | A/G |
| 2 | 29242383 | G/A |
| 2 | 29242384 | G/T |
| 2 | 29242385 | G/T |
| 2 | 29242386 | G/T |
| 2 | 29297701 | C/T |
| 2 | 29301643 | A/G |
| 2 | 29321653 | A/G |
| 2 | 29351825 | G/A |
| 2 | 29362920 | A/G |
| 2 | 29385892 | A/G |
| 2 | 29395522 | G/A |
| 2 | 29401538 | G/A |
| 2 | 29448741 | G/T |
| 2 | 29449992 | G/A |
| 2 | 29464023 | T/G |
| 2 | 29471522 | C/T |
| 2 | 29514777 | A/G |
| 2 | 29521810 | G/T |
| 2 | 29551336 | T/C |
| 2 | 29573270 | G/A |
| 2 | 29556755 | T/G |
| 2 | 29604518 | C/A |
| 2 | 29628386 | G/A |
| 2 | 39643401 | T/C |
| 2 | 29648740 | G/C |
| 2 | 29680946 | T/A |
| 2 | 29708379 | C/T |
| 2 | 29710632 | G/T |
| 2 | 29736289 | C/T |
| 2 | 29756136 | G/A |
| 2 | 29772267 | T/C |
| 2 | 29775534 | T/C |
| 2 | 29790524 | G/T |
| 2 | 29828487 | G/A |
| 2 | 29851184 | G/A |
| 2 | 29853010 | A/G |
| 2 | 29880555 | T/C |
| 2 | 29890635 | C/T |
| 2 | 29910864 | T/C |
| 2 | 29968668 | G/A |
| 2 | 29971579 | A/G |
| 2 | 29986101 | G/A |
| 2 | 30000471 | A/C |
| 2 | 30049380 | A/G |
| 2 | 30080963 | C/G |
| 2 | 30085925 | C/A |
| 2 | 30103932 | A/G |
| 2 | 30114377 | A/C |
| 2 | 30127618 | A/C |
| 2 | 30173657 | T/C |
| 2 | 30175020 | A/G |
| 2 | 30182821 | A/G |
| 2 | 30202587 | C/G |
| 2 | 30213903 | T/G |
| 2 | 30213904 | C/T |
| 2 | 30213905 | C/T |
| 2 | 30213906 | C/T |
| 2 | 30285697 | G/T |
| 2 | 30353084 | T/G |
| 2 | 30371276 | C/G |
| 2 | 30431562 | T/C |
| 2 | 30434580 | C/G |
| 2 | 30463997 | T/C |
| 2 | 30494242 | C/T |
| 2 | 30506379 | A/C |
| 2 | 30516066 | G/A |
| 2 | 30520443 | G/A |
| 2 | 30543833 | A/G |
| 2 | 30568645 | T/C |
| 2 | 30586973 | T/C |
| 2 | 30595661 | G/T |
| 2 | 30595662 | C/T |
| 2 | 30595663 | C/T |
| 2 | 30637943 | G/A |
| 2 | 30657562 | C/T |
| 2 | 30681131 | G/A |
| 2 | 30719986 | C/T |
| 2 | 30748370 | G/C |
| 2 | 30749942 | A/G |
| 2 | 30752957 | G/A |
| 2 | 30755669 | T/C |
| 2 | 30783616 | G/A |
| 2 | 30807686 | T/C |
| 2 | 30831710 | T/A |
| 2 | 30841424 | G/T |
| 2 | 30886007 | T/A |
| 2 | 30929413 | G/A |
| 2 | 30970521 | A/C |
| 2 | 30975890 | A/G |
| 2 | 30975891 | T/A |
| 2 | 30975892 | T/A |
| 2 | 30975893 | T/A |
| 2 | 31023850 | G/A |
| 2 | 31053986 | G/T |
| 2 | 31100138 | A/G |
| 2 | 31106588 | A/G |
| 2 | 31125507 | C/T |
| 2 | 31139779 | T/C |
| 2 | 31143426 | C/T |
| 2 | 31172633 | C/T |
| 2 | 31172634 | C/T |
| 2 | 31172635 | C/T |
| 2 | 31172636 | C/T |
| 2 | 31247471 | C/T |
| 2 | 31589187 | T/G |
| 2 | 31273670 | T/C |
| 2 | 31275001 | T/A |
| 2 | 31295682 | C/G |
| 2 | 31333032 | C/T |
| 2 | 31340655 | A/G |
| 2 | 31370628 | G/A |
| 2 | 31375446 | A/C |
| 2 | 31403878 | G/T |
| 2 | 31413274 | C/T |
| 2 | 31417258 | C/T |
| 2 | 31466052 | T/C |
| 2 | 31470463 | G/A |
| 2 | 31501833 | T/A |
| 2 | 31536690 | C/G |
| 2 | 31563553 | T/C |
| 2 | 31571282 | C/T |
| 2 | 31571283 | C/T |
| 2 | 31571284 | C/T |
| 2 | 31571285 | C/T |
| 2 | 31571286 | G/A |
| 2 | 31571287 | G/A |
| 2 | 31571288 | G/A |
| 2 | 31683889 | T/C |
| 2 | 31729941 | T/C |
| 2 | 31750347 | A/G |
| 2 | 31769723 | T/C |
| 2 | 31771584 | G/C |
| 2 | 31771585 | G/A |
| 2 | 31771586 | G/A |
| 2 | 31771587 | G/A |
| 2 | 31840793 | G/A |
| 2 | 31848094 | C/T |
| 2 | 31862308 | G/A |
| 2 | 31868693 | C/T |
| 2 | 31905525 | T/G |
| 2 | 31909672 | T/C |
| 2 | 31931433 | G/A |
| 2 | 31935929 | G/A |
| 2 | 31950117 | T/C |
| 2 | 32009920 | T/A |
| 2 | 32013049 | G/A |
| 2 | 32023663 | T/C |
| 2 | 32048079 | T/A |
| 2 | 32086481 | G/A |
| 2 | 32091818 | T/C |
| 2 | 32100552 | C/T |
| 2 | 32130648 | T/C |
| 2 | 32131934 | T/A |
| 2 | 32131935 | G/A |
| 2 | 32131936 | G/A |
| 2 | 32131937 | G/A |
| 2 | 32218322 | C/T |
| 2 | 32228949 | A/G |
| 2 | 32286975 | T/A |
| 2 | 32286976 | G/A |
| 2 | 32286977 | G/A |
| 2 | 32286978 | G/A |
| 2 | 32345998 | C/G |
| 2 | 32367832 | C/G |
| 2 | 32389987 | G/A |
| 2 | 32399688 | G/T |
| 2 | 32412949 | T/A |
| 2 | 32429885 | G/C |
| 2 | 32450860 | A/G |
| 2 | 32467227 | G/A |
| 2 | 32470933 | T/C |
| 2 | 32505718 | A/C |
| 2 | 32518348 | C/T |
| 2 | 32528548 | A/G |
| 2 | 32553251 | A/G |
| 2 | 32571888 | C/T |
| 2 | 32584525 | A/C |
| 2 | 32603641 | T/G |
| 2 | 32628147 | G/A |
| 2 | 32643399 | T/C |
| 2 | 32675981 | A/G |
| 2 | 32678210 | A/G |
| 2 | 32703960 | A/G |
| 2 | 32737889 | T/C |
| 2 | 32767575 | G/A |
| 2 | 32792053 | A/C |
| 2 | 32816746 | G/T |
| 2 | 32823603 | A/G |
| 2 | 32839849 | T/C |
| 2 | 32857002 | A/C |
| 2 | 32897971 | A/C |
| 2 | 32912752 | C/T |
| 2 | 32915045 | G/T |
| 2 | 32949472 | T/C |
| 2 | 32954369 | T/A |
| 2 | 32971770 | C/A |
| 2 | 32978056 | G/A |
| 2 | 33024081 | A/G |
| 2 | 33030868 | T/A |
| 2 | 33089707 | A/G |
| 2 | 33111242 | G/A |
| 2 | 33119262 | A/G |
| 2 | 33135103 | C/T |
| 2 | 33204944 | C/T |
| 2 | 33206255 | A/G |
| 2 | 33214259 | A/G |
| 2 | 33254323 | T/C |
| 2 | 33260023 | C/T |
| 2 | 33277086 | A/C |
| 2 | 33302085 | T/C |
| 2 | 33320495 | A/G |
| 2 | 33331574 | A/T |
| 2 | 33348986 | T/A |
| 2 | 33364946 | C/T |
| 2 | 33392187 | A/G |
| 2 | 33397725 | A/G |
| 2 | 33441216 | C/T |
| 2 | 33472313 | C/T |
| 2 | 33478815 | A/T |
| 2 | 33484687 | C/T |
| 2 | 33499663 | G/C |
| 2 | 33523944 | G/C |
| 2 | 33525549 | T/C |
| 2 | 33344237 | C/T |
| 2 | 33570332 | A/G |
| 2 | 33606470 | T/G |
| 2 | 33630907 | T/C |
| 2 | 33632573 | C/T |
| 2 | 33660452 | C/T |
| 2 | 33672652 | G/A |
| 2 | 33706710 | T/C |
| 2 | 33708132 | T/C |
| 2 | 33739560 | A/T |
| 2 | 33745519 | T/G |
| 2 | 33750435 | A/G |
| 2 | 33757261 | G/A |
| 2 | 33807642 | T/A |
| 2 | 33826184 | A/G |
| 2 | 33840199 | A/C |
| 2 | 33843915 | T/C |
| 2 | 33843916 | C/T |
| 2 | 33843917 | C/T |
| 2 | 33843918 | C/T |
| 2 | 33913401 | C/T |
| 2 | 33929150 | G/T |
| 2 | 33937694 | A/T |
| 2 | 33947052 | T/G |
| 2 | 33975716 | C/T |
| 2 | 33978720 | T/G |
| 2 | 33990843 | C/A |
| 2 | 34030573 | G/T |
| 2 | 34054274 | C/T |
| 2 | 34059490 | A/C |
| 2 | 34084499 | G/A |
| 2 | 34096681 | A/G |
| 2 | 34126316 | G/C |
| 2 | 34130150 | G/A |
| 2 | 34147183 | G/A |
| 2 | 34176931 | G/A |
| 2 | 34180985 | C/G |
| 2 | 34216787 | C/T |
| 2 | 34225084 | A/G |
| 2 | 34251943 | T/C |
| 2 | 34257317 | T/C |
| 2 | 34264463 | T/G |
| 2 | 34294337 | T/A |
| 2 | 34321846 | G/A |
| 2 | 34323590 | T/C |
| 2 | 34377835 | A/G |
| 2 | 34399524 | A/G |
| 2 | 34411984 | T/G |
| 2 | 34415632 | G/A |
| 2 | 34442460 | G/A |
| 2 | 34473096 | G/A |
| 2 | 34510052 | C/T |
| 2 | 34523077 | T/C |
| 2 | 34539076 | T/C |
| 2 | 34589705 | T/G |
| 2 | 34600665 | C/T |
| 2 | 34627307 | A/G |
| 2 | 34648298 | G/A |
| 2 | 34657507 | G/A |
| 2 | 34699579 | G/A |
| 2 | 34703854 | T/C |
| 2 | 34714948 | C/A |
| 2 | 34716123 | T/A |
| 2 | 34749467 | G/T |
| 2 | 34752110 | A/C |
| 2 | 34803157 | A/G |
| 2 | 34869358 | G/C |
| 2 | 35086555 | A/T |
| 2 | 35092998 | T/G |
| 2 | 35126147 | A/G |
| 2 | 35134106 | C/T |
| 2 | 35164244 | G/A |
| 2 | 35181555 | G/A |
| 2 | 35193150 | G/A |
| 2 | 35236982 | G/A |
| 2 | 35247726 | C/T |
| 2 | 35253140 | A/G |
| 2 | 35256525 | G/C |
| 2 | 35294403 | T/C |
| 2 | 35320679 | A/G |
| 2 | 35333528 | T/C |
| 2 | 35366584 | T/C |
| 2 | 35375710 | T/G |
| 2 | 35419295 | G/A |
| 2 | 35422163 | C/T |
| 2 | 35457659 | C/T |
| 2 | 35478455 | G/A |
| 2 | 35481659 | A/G |
| 2 | 35496713 | T/A |
| 2 | 35524518 | T/G |
| 2 | 35609575 | G/T |
| 2 | 35613163 | G/A |
| 2 | 35644331 | T/C |
| 2 | 35652987 | T/C |
| 2 | 35690212 | T/C |
| 2 | 35705458 | T/G |
| 2 | 35711137 | C/T |
| 2 | 35735162 | C/G |
| 2 | 35749592 | C/T |
| 2 | 35772279 | C/A |
| 2 | 35791305 | A/G |
| 2 | 35823072 | T/C |
| 2 | 35869745 | G/T |
| 2 | 35913295 | T/C |
| 2 | 35920065 | A/G |
| 2 | 35955905 | T/C |
| 2 | 35994499 | G/C |
| 2 | 35996054 | C/T |
| 2 | 36029830 | C/T |
| 2 | 36038464 | C/T |
| 2 | 36145329 | A/C |
| 2 | 36298748 | C/T |
| 2 | 36319982 | G/A |
| 2 | 36352956 | T/C |
| 2 | 36354098 | C/T |
| 2 | 36369686 | A/G |
| 2 | 36389668 | A/T |
| 2 | 36396243 | A/G |
| 2 | 36418687 | C/T |
| 2 | 36423601 | C/T |
| 2 | 36428243 | G/A |
| 2 | 36460236 | G/A |
| 2 | 36481591 | A/G |
| 2 | 36511593 | T/C |
| 2 | 36530525 | T/C |
| 2 | 36666435 | C/T |
| 2 | 36708962 | A/G |
| 2 | 36716254 | G/C |
| 2 | 36721154 | C/G |
| 2 | 36749160 | C/T |
| 2 | 36776666 | G/T |
| 2 | 36787022 | C/T |
| 2 | 36788106 | G/A |
| 2 | 36813026 | G/A |
| 2 | 36837108 | G/A |
| 2 | 36847447 | C/T |
| 2 | 36852359 | T/C |
| 2 | 36877233 | G/C |
| 2 | 36891954 | T/A |
| 2 | 36922606 | T/A |
| 2 | 36953396 | T/C |
| 2 | 36959887 | C/T |
| 2 | 36961841 | G/A |
| 2 | 36976767 | A/G |
| 2 | 36993171 | C/T |
| 2 | 37012459 | T/C |
| 2 | 37030454 | C/G |
| 2 | 37049972 | G/A |
| 2 | 37062008 | A/G |
| 2 | 37126656 | C/T |
| 2 | 37127831 | G/A |
| 2 | 37153293 | A/G |
| 2 | 37155434 | G/A |
| 2 | 37180522 | G/A |
| 2 | 37208784 | A/G |
| 2 | 37238191 | T/C |
| 2 | 37263829 | C/T |
| 2 | 37277361 | G/C |
| 2 | 37294745 | T/C |
| 2 | 37297793 | C/A |
| 2 | 37337329 | G/A |
| 2 | 37367401 | C/T |
| 2 | 37379013 | T/C |
| 2 | 37388269 | A/C |
| 2 | 37398204 | C/T |
| 2 | 37421508 | G/A |
| 2 | 37450651 | T/C |
| 2 | 37454305 | G/T |
| 2 | 37475737 | C/T |
| 2 | 37510767 | A/G |
| 2 | 37510768 | G/A |
| 2 | 37510769 | G/A |
| 2 | 37510770 | G/A |
| 2 | 37571823 | C/T |
| 2 | 37572916 | T/C |
| 2 | 37615043 | G/A |
| 2 | 37638370 | T/C |
| 2 | 37667724 | G/T |
| 2 | 37676474 | T/A |
| 2 | 37680055 | G/C |
| 2 | 37717808 | A/G |
| 2 | 37725882 | G/A |
| 2 | 37754278 | C/T |
| 2 | 37772517 | C/T |
| 2 | 37826279 | T/A |
| 2 | 37853210 | A/G |
| 2 | 37856628 | C/A |
| 2 | 37872324 | T/C |
| 2 | 37913445 | G/A |
| 2 | 37922916 | C/T |
| 2 | 37928552 | A/G |
| 2 | 37951281 | G/C |
| 2 | 37957767 | C/T |
| 2 | 38010951 | T/C |
| 2 | 38014620 | T/C |
| 2 | 38151459 | G/A |
| 2 | 38237693 | G/A |
| 2 | 38242384 | A/G |
| 2 | 38278718 | G/A |
| 2 | 38289618 | G/A |
| 2 | 38325509 | T/C |
| 2 | 38360255 | G/C |
| 2 | 38389351 | G/A |
| 2 | 38390433 | G/A |
| 2 | 38403501 | A/T |
| 2 | 38448834 | G/A |
| 2 | 38464923 | T/C |
| 2 | 38467377 | A/C |
| 2 | 38501049 | G/A |
| 2 | 38521944 | G/T |
| 2 | 38541277 | A/G |
| 2 | 38562499 | C/T |
| 2 | 38565553 | T/C |
| 2 | 38594436 | T/C |
| 2 | 38619465 | A/G |
| 2 | 38712574 | G/A |
| 2 | 38728243 | C/G |
| 2 | 38732377 | A/G |
| 2 | 38786260 | C/A |
| 2 | 38787628 | A/G |
| 2 | 38798015 | C/T |
| 2 | 38803051 | C/T |
| 2 | 38824998 | G/A |
| 2 | 38850639 | C/T |
| 2 | 38893390 | G/A |
| 2 | 38894494 | T/C |
| 2 | 38915515 | G/A |
| 2 | 38953363 | G/A |
| 2 | 38971414 | C/G |
| 2 | 38974366 | A/G |
| 2 | 39010886 | T/G |
| 2 | 39049889 | T/G |
| 2 | 39086673 | T/C |
| 2 | 39106485 | C/T |
| 2 | 39112331 | C/T |
| 2 | 39146845 | C/G |
| 2 | 39149931 | T/C |
| 2 | 39152188 | G/A |
| 2 | 39155113 | C/G |
| 2 | 39191191 | T/G |
| 2 | 39192342 | A/C |
| 2 | 39219963 | T/C |
| 2 | 39224838 | T/G |
| 2 | 39249777 | G/T |
| 2 | 39282191 | C/T |
| 2 | 39304381 | G/A |
| 2 | 39306940 | A/T |
| 2 | 39350122 | A/G |
| 2 | 39356070 | A/G |
| 2 | 39365592 | C/T |
| 2 | 39398525 | G/A |
| 2 | 39416634 | G/T |
| 2 | 39423474 | G/A |
| 2 | 39446030 | G/C |
| 2 | 39452651 | G/C |
| 2 | 39453914 | T/A |
| 2 | 39502877 | G/T |
| 2 | 39516326 | C/T |
| 2 | 39557543 | G/A |
| 2 | 39569502 | G/T |
| 2 | 39573945 | T/C |
| 2 | 39611424 | C/A |
| 2 | 39653728 | C/T |
| 2 | 39660478 | C/T |
| 2 | 39690208 | G/A |
| 2 | 39701782 | G/C |
| 2 | 39758302 | C/T |
| 2 | 39778061 | C/T |
| 2 | 39783175 | C/T |
| 2 | 39801615 | T/C |
| 2 | 39801616 | G/A |
| 2 | 39843049 | G/A |
| 2 | 39879023 | A/C |
| 2 | 39905303 | C/T |
| 2 | 39923922 | A/G |
| 2 | 39938748 | T/C |
| 2 | 39964322 | A/T |
| 2 | 39979450 | G/A |
| 2 | 40000050 | C/A |
| 2 | 40035888 | C/G |
| 2 | 40043681 | A/G |
| 2 | 40064689 | T/C |
| 2 | 40074860 | A/C |
| 2 | 40085914 | C/T |
| 2 | 40116136 | T/G |
| 2 | 40118658 | T/C |
| 2 | 40168610 | C/T |
| 2 | 40169872 | G/A |
| 2 | 40187648 | A/G |
| 2 | 40208775 | G/A |
| 2 | 40218062 | T/A |
| 2 | 40249311 | C/G |
| 2 | 40261186 | T/C |
| 2 | 40312148 | T/C |
| 2 | 40326153 | C/T |
| 2 | 40356045 | T/C |
| 2 | 40370989 | A/G |
| 2 | 40380042 | C/A |
| 2 | 40382310 | T/C |
| 2 | 40414235 | C/A |
| 2 | 40421630 | G/C |
| 2 | 40443318 | A/G |
| 2 | 40464292 | A/G |
| 2 | 40482267 | C/T |
| 2 | 40494355 | T/C |
| 2 | 40509301 | G/A |
| 2 | 40514440 | G/A |
| 2 | 40538765 | T/C |
| 2 | 40555114 | A/T |
| 2 | 40572418 | A/G |
| 2 | 40574774 | T/G |
| 2 | 40610320 | C/T |
| 2 | 40619509 | G/A |
| 2 | 40630921 | C/G |
| 2 | 40670811 | T/C |
| 2 | 40672882 | G/A |
| 2 | 40684973 | G/A |
| 2 | 40686738 | G/A |
| 2 | 40686739 | G/C |
| 2 | 40686740 | G/C |
| 2 | 40686741 | G/C |
| 2 | 40748785 | C/G |
| 2 | 40762016 | G/A |
| 2 | 40785571 | A/G |
| 2 | 40788009 | C/T |
| 2 | 40820336 | T/G |
| 2 | 40833638 | G/T |
| 2 | 40882037 | T/C |
| 2 | 40900188 | T/A |
| 2 | 40932663 | A/G |
| 2 | 40934351 | C/T |
| 2 | 40966797 | C/T |
| 2 | 40979002 | A/C |
| 2 | 40995854 | C/A |
| 2 | 41000025 | T/A |
| 2 | 41010348 | T/C |
| 2 | 41033424 | C/T |
| 2 | 41065810 | T/C |
| 2 | 41071004 | G/T |
| 2 | 41083289 | A/G |
| 2 | 41100738 | T/G |
| 2 | 41126150 | T/C |
| 2 | 41150833 | T/C |
| 2 | 41165258 | G/A |
| 2 | 41169064 | T/C |
| 2 | 41210661 | C/T |
| 2 | 41211768 | T/A |
| 2 | 41225944 | G/A |
| 2 | 41227117 | C/T |
| 2 | 41253465 | T/C |
| 2 | 41258407 | A/G |
| 2 | 41287202 | A/T |
| 2 | 41315461 | C/A |
| 2 | 41341520 | G/A |
| 2 | 41365063 | T/C |
| 2 | 41370128 | T/C |
| 2 | 41371342 | T/A |
| 2 | 41429571 | G/A |
| 2 | 41450833 | C/G |
| 2 | 41459606 | G/A |
| 2 | 41463197 | C/A |
| 2 | 41490659 | C/T |
| 2 | 41520380 | C/A |
| 2 | 41528307 | A/G |
| 2 | 41550209 | G/A |
| 2 | 41572594 | A/G |
| 2 | 41590726 | C/T |
| 2 | 41599051 | G/A |
| 2 | 41628455 | C/A |
| 2 | 41637950 | T/C |
| 2 | 41641322 | G/A |
| 2 | 41657839 | G/C |
| 2 | 41671686 | G/A |
| 2 | 41713603 | G/A |
| 2 | 41744668 | C/T |
| 2 | 41750128 | A/G |
| 2 | 41760049 | G/C |
| 2 | 41811471 | C/T |
| 2 | 41814335 | C/T |
| 2 | 41829058 | C/T |
| 2 | 41835439 | G/C |
| 2 | 41851957 | T/G |
| 2 | 41883773 | C/G |
| 2 | 41918838 | G/A |
| 2 | 41939706 | G/T |
| 2 | 41941025 | T/C |
| 2 | 41971920 | G/A |
| 2 | 41994485 | T/C |
| 2 | 42014944 | G/A |
| 2 | 42020511 | G/A |
| 2 | 42033686 | G/A |
| 2 | 42059346 | G/C |
| 2 | 42074344 | G/A |
| 2 | 42082418 | G/T |
| 2 | 42093978 | G/A |
| 2 | 42121993 | T/C |
| 2 | 42148860 | C/T |
| 2 | 42158225 | C/G |
| 2 | 42206663 | G/T |
| 2 | 42222446 | T/C |
| 2 | 42240523 | C/A |
| 2 | 42279184 | A/G |
| 2 | 42319501 | G/A |
| 2 | 42321684 | G/A |
| 2 | 42353477 | G/A |
| 2 | 42362743 | C/T |
| 2 | 42394046 | C/T |
| 2 | 42408307 | C/T |
| 2 | 42436106 | T/C |
| 2 | 42453388 | C/A |
| 2 | 42483847 | G/A |
| 2 | 42514711 | G/A |
| 2 | 42522779 | T/C |
| 2 | 42546824 | A/G |
| 2 | 42588103 | C/G |
| 2 | 42612091 | G/T |
| 2 | 42623100 | G/T |
| 2 | 42631565 | A/T |
| 2 | 42637129 | C/T |
| 2 | 42673138 | A/G |
| 2 | 42676078 | C/T |
| 2 | 42697376 | T/C |
| 2 | 42719922 | A/T |
| 2 | 42721049 | T/C |
| 2 | 42742693 | T/A |
| 2 | 42763886 | A/G |
| 2 | 42797719 | A/T |
| 2 | 42808181 | A/G |
| 2 | 42816014 | A/G |
| 2 | 42831152 | A/T |
| 2 | 42862569 | G/A |
| 2 | 42879823 | T/C |
| 2 | 42894124 | A/G |
| 2 | 42908113 | C/A |
| 2 | 42914602 | T/C |
| 2 | 42943531 | C/A |
| 2 | 42947955 | G/A |
| 2 | 42960304 | T/A |
| 2 | 43007517 | A/G |
| 2 | 43022603 | G/A |
| 2 | 43025923 | A/C |
| 2 | 43059098 | T/C |
| 2 | 43094905 | T/A |
| 2 | 43132648 | G/A |
| 2 | 43152690 | A/T |
| 2 | 43156736 | A/G |
| 2 | 43186917 | A/G |
| 2 | 43210815 | G/A |
| 2 | 43223339 | T/C |
| 2 | 43239426 | A/G |
| 2 | 43275911 | G/T |
| 2 | 43288344 | G/A |
| 2 | 43312033 | G/A |
| 2 | 43321456 | T/C |
| 2 | 43341329 | A/G |
| 2 | 43367841 | T/C |
| 2 | 43380050 | A/G |
| 2 | 43423406 | A/C |
| 2 | 43433202 | T/G |
| 2 | 43440330 | T/C |
| 2 | 43475726 | G/A |
| 2 | 43481874 | C/T |
| 2 | 43509338 | C/A |
| 2 | 43512376 | G/T |
| 2 | 43551523 | C/T |
| 2 | 43558078 | C/T |
| 2 | 43566306 | A/C |
| 2 | 43577021 | G/A |
| 2 | 43592581 | A/G |
| 2 | 43600688 | A/G |
| 2 | 43642583 | G/A |
| 2 | 43676913 | A/G |
| 2 | 43696000 | C/T |
| 2 | 43719816 | T/C |
| 2 | 43723533 | C/T |
| 2 | 43746506 | T/C |
| 2 | 43808798 | G/A |
| 2 | 43857104 | A/G |
| 2 | 43875832 | C/T |
| 2 | 43906565 | T/C |
| 2 | 43917985 | G/A |
| 2 | 43922291 | T/A |
| 2 | 43943355 | T/C |
| 2 | 43953949 | G/A |
| 2 | 43989935 | C/T |
| 2 | 44012112 | A/G |
| 2 | 44030886 | C/A |
| 2 | 44053514 | G/A |
| 2 | 44070241 | A/G |
| 2 | 44084100 | C/T |
| 2 | 44100226 | C/T |
| 2 | 44102885 | G/T |
| 2 | 44151673 | C/T |
| 2 | 44171914 | C/A |
| 2 | 44177404 | T/C |
| 2 | 44213038 | T/C |
| 2 | 44221619 | C/T |
| 2 | 44246067 | T/A |
| 2 | 44270058 | G/A |
| 2 | 44273927 | A/T |
| 2 | 44294021 | C/G |
| 2 | 44315371 | G/T |
| 2 | 44367867 | C/T |
| 2 | 44369776 | T/G |
| 2 | 44376247 | T/C |
| 2 | 44378537 | G/A |
| 2 | 44401394 | A/C |
| 2 | 44515642 | G/A |
| 2 | 44542131 | A/G |
| 2 | 44565690 | G/A |
| 2 | 44600387 | G/A |
| 2 | 44611336 | A/T |
| 2 | 44615280 | G/A |
| 2 | 44666196 | G/A |
| 2 | 44682121 | T/A |
| 2 | 44709290 | G/A |
| 2 | 44722343 | A/G |
| 2 | 44730322 | A/G |
| 2 | 44735095 | T/G |
| 2 | 44765261 | G/A |
| 2 | 44766794 | C/G |
| 2 | 44801076 | T/C |
| 2 | 44803578 | G/A |
| 2 | 44820381 | T/C |
| 2 | 44826119 | T/A |
| 2 | 44857586 | G/T |
| 2 | 44909200 | A/G |
| 2 | 44914962 | C/T |
| 2 | 44927380 | C/A |
| 2 | 44947297 | G/T |
| 2 | 44965759 | G/A |
| 2 | 45018981 | A/C |
| 2 | 45038287 | G/A |
| 2 | 45073737 | A/G |
| 2 | 45098472 | G/A |
| 2 | 45124676 | C/A |
| 2 | 45146254 | G/A |
| 2 | 45161771 | G/A |
| 2 | 45195054 | A/T |
| 2 | 45196212 | C/G |
| 2 | 45237451 | C/T |
| 2 | 45237452 | G/A |
| 2 | 45237453 | G/A |
| 2 | 45237454 | G/A |
| 2 | 45314319 | C/T |
| 2 | 45354564 | T/C |
| 2 | 45356258 | C/T |
| 2 | 45366579 | A/G |
| 2 | 45401599 | C/T |
| 2 | 45417960 | G/A |
| 2 | 45420254 | C/T |
| 2 | 45464088 | A/G |
| 2 | 45484148 | T/A |
| 2 | 45498029 | C/T |
| 2 | 45511787 | T/A |
| 2 | 45521339 | T/C |
| 2 | 45541910 | T/C |
| 2 | 45569358 | C/A |
| 2 | 45619376 | T/A |
| 2 | 45623082 | C/T |
| 2 | 45630067 | C/T |
| 2 | 45656224 | A/G |
| 2 | 45665249 | C/T |
| 2 | 45679054 | C/A |
| 2 | 45705911 | G/T |
| 2 | 45723945 | A/T |
| 2 | 45758285 | G/T |
| 2 | 45761119 | C/T |
| 2 | 45784254 | T/C |
| 2 | 45790736 | G/C |
| 2 | 45827804 | G/A |
| 2 | 45866178 | T/G |
| 2 | 45879995 | G/A |
| 2 | 45881483 | G/A |
| 2 | 45909759 | T/A |
| 2 | 45941197 | G/A |
| 2 | 45949512 | C/T |
| 2 | 45961276 | A/G |
| 2 | 45997768 | T/A |
| 2 | 46004065 | T/A |
| 2 | 46033745 | T/C |
| 2 | 46048360 | C/A |
| 2 | 46055680 | A/G |
| 2 | 46059810 | C/T |
| 2 | 46093172 | C/T |
| 2 | 46174314 | A/G |
| 2 | 46193166 | A/G |
| 2 | 46209791 | A/G |
| 2 | 46234521 | A/T |
| 2 | 46293721 | T/A |
| 2 | 46319879 | A/C |
| 2 | 46327152 | T/A |
| 2 | 46346859 | C/T |
| 2 | 46367203 | G/C |
| 2 | 46445075 | G/A |
| 2 | 46471438 | A/G |
| 2 | 46532946 | G/A |
| 2 | 46588207 | A/G |
| 2 | 46610889 | C/T |
| 2 | 46625817 | T/G |
| 2 | 46643568 | C/A |
| 2 | 46654688 | A/T |
| 2 | 46656040 | C/A |
| 2 | 46680143 | G/A |
| 2 | 46687773 | C/T |
| 2 | 46710422 | C/T |
| 2 | 46760155 | C/T |
| 2 | 46774506 | T/C |
| 2 | 46794961 | T/A |
| 2 | 46807265 | G/T |
| 2 | 46816364 | T/C |
| 2 | 46855324 | T/G |
| 2 | 46873534 | G/C |
| 2 | 46878341 | G/A |
| 2 | 46895580 | C/T |
| 2 | 46921615 | G/A |
| 2 | 46953508 | C/A |
| 2 | 46991123 | T/C |
| 2 | 47022675 | T/A |
| 2 | 47046777 | T/C |
| 2 | 47051884 | C/T |
| 2 | 47090014 | C/T |
| 2 | 47100407 | T/C |
| 2 | 47101870 | G/A |
| 2 | 47131553 | G/A |
| 2 | 47163286 | G/A |
| 2 | 47188002 | T/C |
| 2 | 47200304 | G/A |
| 2 | 47248975 | A/T |
| 2 | 47250312 | T/A |
| 2 | 47256474 | T/C |
| 2 | 47281991 | G/A |
| 2 | 47286296 | C/T |
| 2 | 47323221 | C/T |
| 2 | 47332007 | C/G |
| 2 | 47367035 | A/G |
| 2 | 47388908 | C/T |
| 2 | 47412256 | G/T |
| 2 | 47437822 | A/C |
| 2 | 47462838 | C/G |
| 2 | 47493939 | G/A |
| 2 | 47496032 | T/C |
| 2 | 47542883 | C/G |
| 2 | 47353333 | T/C |
| 2 | 47554803 | A/T |
| 2 | 47588954 | A/C |
| 2 | 47628799 | C/T |
| 2 | 47646696 | C/A |
| 2 | 47648820 | G/A |
| 2 | 47694054 | C/T |
| 2 | 47721929 | T/A |
| 2 | 47729446 | A/G |
| 2 | 47741339 | T/G |
| 2 | 47759071 | G/C |
| 2 | 47760922 | G/A |
| 2 | 47773055 | C/T |
| 2 | 47790273 | G/T |
| 2 | 47823390 | G/A |
| 2 | 47825851 | C/G |
| 2 | 47850307 | T/G |
| 2 | 47853674 | G/C |
| 2 | 47895110 | G/A |
| 2 | 47901153 | G/A |
| 2 | 47934049 | G/T |
| 2 | 47946712 | G/C |
| 2 | 47979626 | A/C |
| 2 | 48007228 | T/A |
| 2 | 48036091 | C/T |
| 2 | 48092387 | C/T |
| 2 | 48117288 | G/A |
| 2 | 48128419 | G/C |
| 2 | 48164479 | C/T |
| 2 | 48178637 | C/T |
| 2 | 48196088 | T/C |
| 2 | 48217679 | G/A |
| 2 | 48223333 | T/C |
| 2 | 48249983 | C/A |
| 2 | 48262052 | T/A |
| 2 | 48282354 | C/G |
| 2 | 48295166 | A/G |
| 2 | 48300306 | G/A |
| 2 | 48302264 | T/C |
| 2 | 48356895 | C/T |
| 2 | 48356896 | G/A |
| 2 | 48356897 | G/A |
| 2 | 48356898 | G/A |
| 2 | 48390688 | A/T |
| 2 | 48437430 | A/T |
| 2 | 48446925 | C/T |
| 2 | 48466543 | G/C |
| 2 | 48481119 | T/A |
| 2 | 48537221 | A/G |
| 2 | 48562706 | A/G |
| 2 | 48566256 | G/A |
| 2 | 48577425 | T/G |
| 2 | 48609325 | C/T |
| 2 | 48630858 | G/T |
| 2 | 48641520 | T/G |
| 2 | 48660144 | T/C |
| 2 | 48705031 | T/C |
| 2 | 48715401 | T/A |
| 2 | 48721712 | G/T |
| 2 | 48771831 | G/A |
| 2 | 48799880 | A/G |
| 2 | 48809166 | G/T |
| 2 | 48810487 | T/C |
| 2 | 48835117 | A/G |
| 2 | 48861172 | G/C |
| 2 | 48870546 | C/G |
| 2 | 48883433 | C/T |
| 2 | 48902149 | T/C |
| 2 | 48905517 | T/A |
| 2 | 48939424 | G/C |
| 2 | 48960218 | C/T |
| 2 | 49006125 | C/T |
| 2 | 49022225 | A/G |
| 2 | 49024975 | G/T |
| 2 | 49053155 | G/T |
| 2 | 49083155 | G/A |
| 2 | 49088882 | G/C |
| 2 | 49110914 | G/A |
| 2 | 49111993 | T/A |
| 2 | 49156425 | G/A |
| 2 | 49157774 | G/T |
| 2 | 49176648 | G/A |
| 2 | 49203116 | T/C |
| 2 | 49216164 | C/T |
| 2 | 49230530 | T/A |
| 2 | 49250455 | G/A |
| 2 | 49261614 | G/A |
| 2 | 49262823 | A/G |
| 2 | 49309708 | C/T |
| 2 | 49313046 | G/A |
| 2 | 49328872 | A/G |
| 2 | 49333019 | G/A |
| 2 | 49377422 | T/G |
| 2 | 49379188 | G/A |
| 2 | 49379189 | G/A |
| 2 | 49408385 | C/T |
| 2 | 49410517 | C/T |
| 2 | 49448639 | A/G |
| 2 | 49467819 | C/G |
| 2 | 49486281 | C/G |
| 2 | 49531949 | G/A |
| 2 | 49543808 | C/A |
| 2 | 49586992 | G/A |
| 2 | 49611487 | A/G |
| 2 | 49613765 | A/G |
| 2 | 49620329 | A/T |
| 2 | 49651997 | G/A |
| 2 | 49658500 | C/T |
| 2 | 49693221 | G/A |
| 2 | 49711819 | G/A |
| 2 | 49752397 | G/A |
| 2 | 49791107 | G/A |
| 2 | 49801037 | C/T |
| 2 | 49838617 | A/G |
| 2 | 49877641 | C/T |
| 2 | 49898752 | T/A |
| 2 | 49911186 | C/T |
| 2 | 49925249 | T/A |
| 2 | 49999216 | G/A |
| 2 | 50108049 | C/T |
| 2 | 50138748 | C/T |
| 2 | 50179249 | G/A |
| 2 | 50187565 | T/C |
| 2 | 50194926 | G/C |
| 2 | 50232529 | T/A |
| 2 | 50274327 | G/A |
| 2 | 50299388 | C/A |
| 2 | 50301949 | G/A |
| 2 | 50330623 | G/A |
| 2 | 50335842 | G/A |
| 2 | 50361885 | G/T |
| 2 | 50366454 | T/A |
| 2 | 50393787 | A/T |
| 2 | 50403645 | T/G |
| 2 | 50428439 | A/G |
| 2 | 50430625 | A/G |
| 2 | 50464824 | C/T |
| 2 | 50517703 | G/A |
| 2 | 50519251 | A/C |
| 2 | 50539276 | A/C |
| 2 | 50558873 | A/C |
| 2 | 50575538 | C/G |
| 2 | 50596915 | A/T |
| 2 | 50627674 | A/G |
| 2 | 50631110 | G/T |
| 2 | 50649063 | A/G |
| 2 | 50678027 | A/G |
| 2 | 50679110 | A/G |
| 2 | 50701298 | G/A |
| 2 | 50746855 | G/A |
| 2 | 50748204 | C/G |
| 2 | 50773860 | C/T |
| 2 | 50781879 | T/C |
| 2 | 50767561 | T/C |
| 2 | 50805791 | C/G |
| 2 | 50828936 | T/G |
| 2 | 50857377 | G/A |
| 2 | 50874873 | T/C |
| 2 | 50882315 | C/T |
| 2 | 50906663 | C/T |
| 2 | 50913232 | T/C |
| 2 | 50922691 | A/T |
| 2 | 50957419 | G/T |
| 2 | 50960673 | C/T |
| 2 | 50982088 | C/T |
| 2 | 50982089 | G/T |
| 2 | 50982090 | G/T |
| 2 | 50982091 | G/T |
| 2 | 51031677 | A/G |
| 2 | 51046621 | C/G |
| 2 | 51074598 | G/A |
| 2 | 51075655 | C/T |
| 2 | 51117679 | C/T |
| 2 | 51141923 | G/C |
| 2 | 51170853 | A/T |
| 2 | 51173500 | C/G |
| 2 | 51183265 | T/C |
| 2 | 51198779 | T/G |
| 2 | 51210248 | C/T |
| 2 | 51240759 | C/A |
| 2 | 51247287 | T/C |
| 2 | 51272761 | T/C |
| 2 | 51332315 | G/C |
| 2 | 51351152 | G/A |
| 2 | 51366674 | C/A |
| 2 | 51371290 | A/C |
| 2 | 51404383 | C/T |
| 2 | 51406343 | T/C |
| 2 | 51424065 | C/A |
| 2 | 51426708 | T/C |
| 2 | 51468267 | C/T |
| 2 | 51480119 | C/A |
| 2 | 51541960 | A/C |
| 2 | 51554407 | C/T |
| 2 | 51572783 | G/T |
| 2 | 51575992 | A/G |
| 2 | 51603451 | C/A |
| 2 | 51606452 | G/A |
| 2 | 51648643 | C/T |
| 2 | 51664564 | T/G |
| 2 | 51666093 | G/A |
| 2 | 51698228 | T/C |
| 2 | 51748153 | C/T |
| 2 | 51750013 | T/C |
| 2 | 51780839 | G/C |
| 2 | 51822155 | T/C |
| 2 | 51843372 | C/A |
| 2 | 51897665 | G/C |
| 2 | 51912251 | T/C |
| 2 | 51955201 | A/T |
| 2 | 51986043 | C/T |
| 2 | 52014121 | G/A |
| 2 | 52019870 | T/C |
| 2 | 52021040 | T/C |
| 2 | 52059117 | A/T |
| 2 | 52062437 | A/C |
| 2 | 52082936 | A/T |
| 2 | 52111132 | C/T |
| 2 | 52142824 | T/C |
| 2 | 52149120 | G/A |
| 2 | 52176074 | G/A |
| 2 | 52194402 | C/T |
| 2 | 52224105 | C/T |
| 2 | 52228984 | A/C |
| 2 | 52230736 | C/T |
| 2 | 52232585 | A/C |
| 2 | 52260364 | T/A |
| 2 | 52268590 | T/C |
| 2 | 52290462 | T/G |
| 2 | 52321822 | G/A |
| 2 | 52344472 | A/G |
| 2 | 52364767 | T/A |
| 2 | 52367127 | C/T |
| 2 | 52384379 | A/G |
| 2 | 52408036 | T/A |
| 2 | 52416163 | C/T |
| 2 | 52420982 | C/T |
| 2 | 52449023 | C/T |
| 2 | 52462752 | C/T |
| 2 | 52471512 | T/G |
| 2 | 52512585 | G/C |
| 2 | 52515663 | C/G |
| 2 | 52548697 | C/A |
| 2 | 52570351 | C/T |
| 2 | 52594926 | A/G |
| 2 | 52603158 | G/A |
| 2 | 52624475 | A/G |
| 2 | 52651427 | C/G |
| 2 | 52656733 | G/A |
| 2 | 52680181 | T/A |
| 2 | 52700714 | A/G |
| 2 | 52712787 | T/C |
| 2 | 52714600 | C/G |
| 2 | 52743716 | C/A |
| 2 | 52771719 | A/G |
| 2 | 52795017 | G/A |
| 2 | 52827935 | A/T |
| 2 | 52839853 | T/C |
| 2 | 52861106 | A/G |
| 2 | 52872836 | G/A |
| 2 | 52892345 | G/A |
| 2 | 52900820 | G/A |
| 2 | 52933107 | C/A |
| 2 | 52943738 | T/A |
| 2 | 52962965 | A/G |
| 2 | 52981722 | G/A |
| 2 | 52996823 | T/C |
| 2 | 53029539 | C/T |
| 2 | 53046309 | C/T |
| 2 | 53047978 | G/A |
| 2 | 53081776 | A/C |
| 2 | 53085647 | C/T |
| 2 | 53101332 | C/T |
| 2 | 53146662 | A/G |
| 2 | 53162094 | A/G |
| 2 | 53194586 | G/A |
| 3 | 51592 | G/A |
| 3 | 61564 | T/C |
| 3 | 111147 | G/T |
| 3 | 219737 | C/A |
| 3 | 296072 | T/C |
| 3 | 314787 | A/G |
| 3 | 316291 | G/A |
| 3 | 351818 | T/C |
| 3 | 385205 | C/T |
| 3 | 387430 | A/G |
| 3 | 390233 | A/G |
| 3 | 395945 | A/G |
| 3 | 444952 | T/C |
| 3 | 450420 | C/T |
| 3 | 481237 | C/T |
| 3 | 517742 | A/T |
| 3 | 528966 | C/T |
| 3 | 567607 | A/T |
| 3 | 569531 | G/C |
| 3 | 573210 | C/T |
| 3 | 582889 | A/G |
| 3 | 604934 | G/A |
| 3 | 641330 | C/T |
| 3 | 674860 | C/T |
| 3 | 722937 | A/G |
| 3 | 734016 | G/A |
| 3 | 773544 | A/C |
| 3 | 780175 | A/G |
| 3 | 806504 | T/C |
| 3 | 812394 | T/C |
| 3 | 826005 | T/A |
| 3 | 848207 | A/G |
| 3 | 863457 | C/T |
| 3 | 874852 | A/T |
| 3 | 899782 | T/C |
| 3 | 901278 | T/C |
| 3 | 902522 | T/A |
| 3 | 937995 | G/A |
| 3 | 988308 | T/G |
| 3 | 1003683 | C/A |
| 3 | 1023781 | C/T |
| 3 | 1071307 | G/C |
| 3 | 1080113 | C/A |
| 3 | 1115061 | T/A |
| 3 | 1122545 | C/T |
| 3 | 1140500 | G/A |
| 3 | 1148216 | C/T |
| 3 | 1183224 | C/T |
| 3 | 1200457 | T/C |
| 3 | 1250692 | G/A |
| 3 | 1271130 | A/G |
| 3 | 1290307 | G/C |
| 3 | 1296031 | G/T |
| 3 | 1320045 | A/G |
| 3 | 1366851 | T/C |
| 3 | 1382615 | T/C |
| 3 | 1424450 | C/T |
| 3 | 1425643 | G/T |
| 3 | 1450514 | C/G |
| 3 | 1457718 | A/T |
| 3 | 1470323 | G/T |
| 3 | 1482879 | G/A |
| 3 | 1503456 | A/G |
| 3 | 1560883 | C/T |
| 3 | 1582702 | G/T |
| 3 | 1642368 | G/A |
| 3 | 1674674 | C/T |
| 3 | 1722700 | T/C |
| 3 | 1760558 | C/A |
| 3 | 1768087 | G/A |
| 3 | 1798820 | T/G |
| 3 | 1810266 | C/G |
| 3 | 1833084 | A/G |
| 3 | 1859810 | C/T |
| 3 | 1862009 | T/C |
| 3 | 1887266 | T/A |
| 3 | 1916166 | C/T |
| 3 | 1917665 | T/C |
| 3 | 1924069 | T/C |
| 3 | 1927543 | T/G |
| 3 | 1950303 | C/G |
| 3 | 2006137 | G/A |
| 3 | 2069684 | G/T |
| 3 | 2092266 | G/A |
| 3 | 2099982 | G/A |
| 3 | 2125450 | C/T |
| 3 | 2149093 | T/A |
| 3 | 2187842 | C/A |
| 3 | 2232478 | A/G |
| 3 | 2258175 | A/G |
| 3 | 2276787 | C/T |
| 3 | 2287019 | T/G |
| 3 | 2306774 | C/G |
| 3 | 2310490 | G/A |
| 3 | 2357818 | C/T |
| 3 | 2365438 | G/A |
| 3 | 2392810 | A/G |
| 3 | 2411238 | C/T |
| 3 | 2430204 | A/G |
| 3 | 2452857 | T/C |
| 3 | 2460432 | T/C |
| 3 | 2491346 | C/A |
| 3 | 2519563 | T/G |
| 3 | 2522088 | C/T |
| 3 | 2546298 | T/C |
| 3 | 2579288 | A/T |
| 3 | 2599166 | T/C |
| 3 | 2607968 | G/C |
| 3 | 2621965 | A/G |
| 3 | 2625509 | G/T |
| 3 | 2681539 | G/C |
| 3 | 2708492 | C/G |
| 3 | 2737398 | C/G |
| 3 | 2761353 | T/C |
| 3 | 2777874 | A/G |
| 3 | 2809466 | G/A |
| 3 | 2816619 | C/A |
| 3 | 2824084 | G/A |
| 3 | 2861953 | G/C |
| 3 | 2891601 | C/T |
| 3 | 2911370 | G/A |
| 3 | 2948205 | T/C |
| 3 | 2976780 | G/A |
| 3 | 2992067 | A/G |
| 3 | 3000301 | T/C |
| 3 | 3034901 | G/A |
| 3 | 3040939 | T/C |
| 3 | 3060205 | G/C |
| 3 | 3064868 | A/C |
| 3 | 3105113 | A/G |
| 3 | 3109814 | A/G |
| 3 | 3130638 | C/T |
| 3 | 3146826 | G/A |
| 3 | 3151723 | C/T |
| 3 | 3180017 | G/A |
| 3 | 3235426 | C/T |
| 3 | 3269860 | T/A |
| 3 | 3301086 | C/T |
| 3 | 3358475 | G/C |
| 3 | 3381337 | A/G |
| 3 | 3393497 | A/G |
| 3 | 3423046 | T/G |
| 3 | 3430847 | A/T |
| 3 | 3453869 | G/T |
| 3 | 3506165 | T/C |
| 3 | 3519487 | C/T |
| 3 | 3521041 | T/C |
| 3 | 3541415 | A/G |
| 3 | 3586443 | G/C |
| 3 | 3590207 | C/G |
| 3 | 3601037 | G/C |
| 3 | 3668029 | T/C |
| 3 | 3709448 | G/A |
| 3 | 3721518 | G/A |
| 3 | 3771330 | T/C |
| 3 | 3787605 | A/T |
| 3 | 3791054 | A/G |
| 3 | 3836610 | G/T |
| 3 | 3900696 | C/T |
| 3 | 3938102 | C/T |
| 3 | 3951269 | C/G |
| 3 | 3968558 | G/T |
| 3 | 3989289 | G/A |
| 3 | 4011598 | C/G |
| 3 | 4042590 | C/A |
| 3 | 4046606 | G/A |
| 3 | 4069589 | A/G |
| 3 | 4095795 | C/T |
| 3 | 4107875 | T/C |
| 3 | 4118767 | C/T |
| 3 | 4127159 | T/G |
| 3 | 4146928 | C/T |
| 3 | 4220836 | G/A |
| 3 | 4250302 | A/G |
| 3 | 4258542 | C/A |
| 3 | 4299676 | C/G |
| 3 | 4344475 | G/C |
| 3 | 4345869 | C/T |
| 3 | 4376793 | C/T |
| 3 | 4378377 | G/C |
| 3 | 4392208 | A/C |
| 3 | 4394064 | T/C |
| 3 | 4411262 | A/G |
| 3 | 4413646 | G/A |
| 3 | 4456978 | C/T |
| 3 | 4468848 | T/C |
| 3 | 4489427 | G/A |
| 3 | 4526570 | T/C |
| 3 | 4535620 | C/T |
| 3 | 4587056 | C/G |
| 3 | 4595237 | G/C |
| 3 | 4602407 | A/G |
| 3 | 4639773 | T/C |
| 3 | 4659729 | C/T |
| 3 | 4664438 | G/A |
| 3 | 4690818 | G/A |
| 3 | 4707166 | G/A |
| 3 | 4735589 | G/A |
| 3 | 4763491 | A/C |
| 3 | 4768974 | C/T |
| 3 | 4772909 | G/T |
| 3 | 4790107 | C/G |
| 3 | 4800100 | C/T |
| 3 | 4848702 | G/A |
| 3 | 4946370 | A/G |
| 3 | 4949630 | A/C |
| 3 | 4960756 | G/A |
| 3 | 4970523 | G/A |
| 3 | 5028501 | T/C |
| 3 | 5038867 | C/A |
| 3 | 5044631 | A/G |
| 3 | 5083995 | A/T |
| 3 | 5091230 | G/A |
| 3 | 5114809 | G/T |
| 3 | 5127963 | G/A |
| 3 | 5131436 | G/A |
| 3 | 5142079 | A/G |
| 3 | 5164964 | G/T |
| 3 | 5200935 | G/T |
| 3 | 5233036 | G/A |
| 3 | 5254031 | C/G |
| 3 | 5302877 | T/G |
| 3 | 5314638 | G/A |
| 3 | 5325506 | G/A |
| 3 | 5342509 | C/T |
| 3 | 5345260 | C/G |
| 3 | 5371871 | C/T |
| 3 | 5408529 | T/C |
| 3 | 5453377 | G/A |
| 3 | 5488717 | G/T |
| 3 | 5492851 | G/A |
| 3 | 5550455 | T/C |
| 3 | 5588098 | G/T |
| 3 | 5591509 | C/A |
| 3 | 5635350 | G/C |
| 3 | 5649519 | C/T |
| 3 | 5654132 | C/T |
| 3 | 5672463 | T/C |
| 3 | 5728755 | A/G |
| 3 | 5734700 | G/A |
| 3 | 5770526 | G/A |
| 3 | 5778451 | C/T |
| 3 | 5795321 | C/T |
| 3 | 5837721 | T/A |
| 3 | 5842565 | C/T |
| 3 | 5905717 | A/G |
| 3 | 5912111 | A/G |
| 3 | 5930000 | G/A |
| 3 | 5974280 | A/G |
| 3 | 6012199 | T/C |
| 3 | 6034896 | C/G |
| 3 | 6052787 | G/A |
| 3 | 6064569 | C/G |
| 3 | 6119523 | G/A |
| 3 | 6123090 | G/A |
| 3 | 6146202 | A/G |
| 3 | 6196578 | G/A |
| 3 | 6198230 | G/A |
| 3 | 6234221 | C/T |
| 3 | 6237617 | G/T |
| 3 | 6262055 | C/T |
| 3 | 6275876 | T/C |
| 3 | 6333416 | G/T |
| 3 | 6372958 | C/G |
| 3 | 6385809 | T/C |
| 3 | 6390138 | A/G |
| 3 | 6403327 | G/A |
| 3 | 6432618 | G/A |
| 3 | 6435446 | A/G |
| 3 | 6450409 | T/C |
| 3 | 6470886 | T/C |
| 3 | 6490150 | T/C |
| 3 | 6498606 | C/T |
| 3 | 6515428 | C/T |
| 3 | 6532967 | G/A |
| 3 | 6561530 | C/T |
| 3 | 6578802 | T/C |
| 3 | 6613105 | A/T |
| 3 | 6616951 | G/C |
| 3 | 6654557 | T/C |
| 3 | 6687786 | G/A |
| 3 | 6737911 | C/T |
| 3 | 6746826 | C/T |
| 3 | 6774594 | A/G |
| 3 | 6786339 | G/C |
| 3 | 6810316 | A/G |
| 3 | 6819051 | A/G |
| 3 | 6840154 | C/A |
| 3 | 6875925 | G/A |
| 3 | 6918861 | C/T |
| 3 | 6943853 | C/T |
| 3 | 6973681 | A/G |
| 3 | 6994019 | G/T |
| 3 | 7026530 | T/C |
| 3 | 7039452 | T/C |
| 3 | 7058640 | C/T |
| 3 | 7086050 | G/A |
| 3 | 7125949 | C/T |
| 3 | 7127869 | T/G |
| 3 | 7149405 | A/G |
| 3 | 7156254 | T/A |
| 3 | 7172381 | T/C |
| 3 | 7201128 | C/T |
| 3 | 7202220 | G/A |
| 3 | 7235975 | T/C |
| 3 | 7272162 | C/T |
| 3 | 7286315 | A/C |
| 3 | 7304016 | T/C |
| 3 | 7328606 | G/A |
| 3 | 7361616 | C/T |
| 3 | 7424419 | G/A |
| 3 | 7498386 | C/T |
| 3 | 7503437 | G/A |
| 3 | 7553823 | G/A |
| 3 | 7577521 | C/T |
| 3 | 7616464 | A/T |
| 3 | 7620097 | G/C |
| 3 | 7623388 | T/C |
| 3 | 7662933 | C/T |
| 3 | 7693416 | C/T |
| 3 | 7710627 | G/C |
| 3 | 7732285 | C/G |
| 3 | 7746382 | A/G |
| 3 | 7756295 | A/G |
| 3 | 7770053 | C/T |
| 3 | 7827846 | T/A |
| 3 | 7833432 | T/C |
| 3 | 7834565 | A/C |
| 3 | 7868842 | C/T |
| 3 | 7899425 | T/C |
| 3 | 7912756 | C/T |
| 3 | 7928896 | G/T |
| 3 | 7936531 | G/T |
| 3 | 7990211 | C/T |
| 3 | 8002364 | G/A |
| 3 | 8011256 | G/C |
| 3 | 8042628 | G/T |
| 3 | 8064905 | A/T |
| 3 | 8084847 | C/A |
| 3 | 8125638 | T/G |
| 3 | 8153076 | G/A |
| 3 | 8160059 | A/G |
| 3 | 8167878 | G/A |
| 3 | 8220400 | A/G |
| 3 | 8244023 | A/G |
| 3 | 8251494 | A/G |
| 3 | 8264230 | C/T |
| 3 | 8282501 | C/T |
| 3 | 8303705 | G/A |
| 3 | 8327518 | G/A |
| 3 | 8344951 | C/T |
| 3 | 8354242 | A/G |
| 3 | 8370466 | C/T |
| 3 | 8416806 | C/T |
| 3 | 8433823 | G/A |
| 3 | 8458708 | G/A |
| 3 | 8502275 | G/A |
| 3 | 8516702 | G/C |
| 3 | 8575739 | A/G |
| 3 | 8580813 | C/T |
| 3 | 8599518 | G/A |
| 3 | 8624638 | G/A |
| 3 | 8639594 | T/C |
| 3 | 9641032 | C/T |
| 3 | 8652636 | C/G |
| 3 | 8681977 | C/G |
| 3 | 8690263 | T/G |
| 3 | 8706940 | T/C |
| 3 | 8712586 | C/T |
| 3 | 8732517 | T/G |
| 3 | 8788074 | C/T |
| 3 | 8795843 | T/C |
| 3 | 8796982 | G/A |
| 3 | 8835337 | C/A |
| 3 | 8836648 | T/C |
| 3 | 8855749 | G/A |
| 3 | 8877223 | C/T |
| 3 | 8891916 | A/G |
| 3 | 8908048 | T/A |
| 3 | 8925802 | G/A |
| 3 | 8946546 | G/A |
| 3 | 8986646 | T/A |
| 3 | 8997936 | A/T |
| 3 | 9004437 | G/A |
| 3 | 9022784 | A/C |
| 3 | 9045452 | A/T |
| 3 | 9050423 | G/A |
| 3 | 9061661 | C/T |
| 3 | 9099151 | G/T |
| 3 | 9137416 | T/C |
| 3 | 9170308 | G/A |
| 3 | 9196246 | C/T |
| 3 | 9198563 | G/C |
| 3 | 9212445 | A/T |
| 3 | 9231675 | G/A |
| 3 | 9240970 | A/C |
| 3 | 9267443 | G/A |
| 3 | 9284765 | G/T |
| 3 | 9295018 | C/G |
| 3 | 9301551 | T/C |
| 3 | 9323571 | T/G |
| 3 | 9352455 | C/G |
| 3 | 9360603 | A/T |
| 3 | 9406171 | G/C |
| 3 | 9419620 | G/A |
| 3 | 9426558 | C/G |
| 3 | 9437627 | G/C |
| 3 | 9487501 | G/C |
| 3 | 9490620 | G/A |
| 3 | 9524614 | A/G |
| 3 | 9543682 | C/T |
| 3 | 9570478 | G/A |
| 3 | 9574726 | G/A |
| 3 | 9605389 | A/C |
| 3 | 9648682 | G/A |
| 3 | 9652377 | A/G |
| 3 | 9668171 | A/T |
| 3 | 9676919 | A/G |
| 3 | 9694981 | A/G |
| 3 | 9717531 | T/C |
| 3 | 9721639 | G/A |
| 3 | 9746512 | T/G |
| 3 | 9755472 | G/C |
| 3 | 9761325 | A/G |
| 3 | 9792948 | G/A |
| 3 | 9815467 | A/G |
| 3 | 9870093 | A/C |
| 3 | 9900555 | C/G |
| 3 | 9934520 | G/A |
| 3 | 9945321 | T/C |
| 3 | 9963080 | T/G |
| 3 | 9998163 | C/T |
| 3 | 10014272 | C/G |
| 3 | 10047199 | A/G |
| 3 | 10058333 | G/A |
| 3 | 10087713 | G/A |
| 3 | 10108708 | G/A |
| 3 | 10117819 | T/C |
| 3 | 10131876 | T/C |
| 3 | 10158912 | C/T |
| 3 | 10169147 | C/A |
| 3 | 10171592 | C/A |
| 3 | 10182026 | T/A |
| 3 | 10202586 | A/G |
| 3 | 10230099 | C/T |
| 3 | 10240091 | C/T |
| 3 | 10275111 | C/G |
| 3 | 10316331 | C/T |
| 3 | 10324082 | T/C |
| 3 | 10326417 | C/A |
| 3 | 10405591 | C/G |
| 3 | 10434614 | T/C |
| 3 | 10439320 | G/T |
| 3 | 10444457 | C/T |
| 3 | 10449938 | C/T |
| 3 | 10477099 | T/A |
| 3 | 10478129 | A/T |
| 3 | 10515926 | G/T |
| 3 | 10552840 | G/A |
| 3 | 10563032 | T/C |
| 3 | 10597973 | G/T |
| 3 | 10612970 | T/C |
| 3 | 10620154 | A/C |
| 3 | 10622885 | T/G |
| 3 | 10675121 | T/C |
| 3 | 10713077 | A/G |
| 3 | 10759786 | A/G |
| 3 | 10768396 | G/A |
| 3 | 10772358 | G/A |
| 3 | 10788088 | A/T |
| 3 | 10815388 | T/C |
| 3 | 10826571 | G/A |
| 3 | 10834332 | C/T |
| 3 | 10886429 | C/T |
| 3 | 10901445 | C/T |
| 3 | 10929942 | C/T |
| 3 | 10943061 | T/G |
| 3 | 10981534 | A/T |
| 3 | 11017494 | C/T |
| 3 | 11043030 | G/A |
| 3 | 11057538 | T/C |
| 3 | 11098691 | T/C |
| 3 | 11102067 | G/A |
| 3 | 11119763 | A/G |
| 3 | 11150423 | T/G |
| 3 | 11179571 | C/G |
| 3 | 11182491 | G/A |
| 3 | 11207248 | A/G |
| 3 | 11218704 | T/C |
| 3 | 11238456 | G/A |
| 3 | 11262161 | G/A |
| 3 | 11292194 | T/C |
| 3 | 11293415 | T/C |
| 3 | 11324913 | C/G |
| 3 | 11337340 | T/C |
| 3 | 11343857 | C/T |
| 3 | 11373285 | G/A |
| 3 | 11384399 | C/G |
| 3 | 11408567 | G/T |
| 3 | 11411522 | C/A |
| 3 | 11437725 | G/A |
| 3 | 11461604 | G/A |
| 3 | 11469787 | T/C |
| 3 | 11497600 | G/T |
| 3 | 11500973 | G/A |
| 3 | 11533484 | C/T |
| 3 | 11544611 | A/G |
| 3 | 11579170 | T/C |
| 3 | 11582508 | T/A |
| 3 | 11609216 | A/G |
| 3 | 11633029 | C/G |
| 3 | 11641329 | G/A |
| 3 | 11653068 | A/G |
| 3 | 11681462 | A/G |
| 3 | 11689606 | G/A |
| 3 | 11701350 | G/A |
| 3 | 11729158 | G/T |
| 3 | 11733462 | C/T |
| 3 | 11776682 | A/C |
| 3 | 11777896 | T/A |
| 3 | 11792737 | G/T |
| 3 | 11806977 | G/C |
| 3 | 11837315 | C/T |
| 3 | 11855609 | T/A |
| 3 | 11868583 | T/A |
| 3 | 11886901 | C/T |
| 3 | 11904438 | T/A |
| 3 | 11910800 | A/C |
| 3 | 11940552 | G/A |
| 3 | 11942844 | C/T |
| 3 | 11972072 | G/T |
| 3 | 11989914 | C/T |
| 3 | 12014916 | G/A |
| 3 | 12032416 | G/C |
| 3 | 12053959 | G/A |
| 3 | 12079380 | G/A |
| 3 | 12083686 | C/A |
| 3 | 12090281 | G/A |
| 3 | 12122004 | T/C |
| 3 | 12123099 | T/A |
| 3 | 12176909 | C/T |
| 3 | 12187584 | G/C |
| 3 | 12207591 | C/T |
| 3 | 12215495 | T/C |
| 3 | 12222917 | C/T |
| 3 | 12254108 | C/T |
| 3 | 12270069 | A/G |
| 3 | 12293553 | C/G |
| 3 | 12307607 | G/A |
| 3 | 12318650 | T/C |
| 3 | 12338278 | C/T |
| 3 | 12361073 | G/A |
| 3 | 12369357 | C/A |
| 3 | 12405855 | G/A |
| 3 | 12412180 | A/G |
| 3 | 12446495 | G/A |
| 3 | 12473326 | G/A |
| 3 | 12492404 | T/C |
| 3 | 12522951 | A/G |
| 3 | 12541440 | A/C |
| 3 | 12596153 | C/T |
| 3 | 12617090 | A/G |
| 3 | 12684419 | G/T |
| 3 | 12711010 | G/A |
| 3 | 12760631 | C/T |
| 3 | 12769919 | C/T |
| 3 | 12785149 | T/G |
| 3 | 12816351 | C/T |
| 3 | 12826037 | A/G |
| 3 | 12841067 | G/A |
| 3 | 12931207 | G/A |
| 3 | 12951150 | C/T |
| 3 | 12981653 | G/A |
| 3 | 13001541 | A/G |
| 3 | 13061592 | G/A |
| 3 | 13096216 | G/A |
| 3 | 13133489 | A/G |
| 3 | 13165736 | C/G |
| 3 | 13171509 | T/C |
| 3 | 13196636 | C/G |
| 3 | 13202164 | T/G |
| 3 | 13231980 | A/G |
| 3 | 13349302 | G/T |
| 3 | 13357143 | G/C |
| 3 | 13390075 | T/G |
| 3 | 13391389 | T/C |
| 3 | 13416070 | C/T |
| 3 | 13425746 | A/G |
| 3 | 13459443 | G/A |
| 3 | 13468896 | C/G |
| 3 | 13498444 | C/T |
| 3 | 13509096 | A/C |
| 3 | 13515174 | G/T |
| 3 | 13531281 | G/A |
| 3 | 13542672 | G/C |
| 3 | 13580338 | T/C |
| 3 | 13589275 | G/A |
| 3 | 13595335 | A/G |
| 3 | 13608265 | G/A |
| 3 | 13646819 | G/A |
| 3 | 13650420 | A/G |
| 3 | 13687988 | C/T |
| 3 | 13700416 | T/A |
| 3 | 13711844 | C/A |
| 3 | 13753313 | G/A |
| 3 | 13762206 | G/A |
| 3 | 13780827 | A/G |
| 3 | 13783705 | C/G |
| 3 | 13809577 | G/C |
| 3 | 13833589 | C/T |
| 3 | 13864745 | A/G |
| 3 | 13912247 | A/C |
| 3 | 13933198 | G/A |
| 3 | 13978548 | C/T |
| 3 | 13979950 | T/C |
| 3 | 13998976 | A/G |
| 3 | 14001093 | T/C |
| 3 | 14020208 | A/T |
| 3 | 14050278 | C/T |
| 3 | 14070102 | G/A |
| 3 | 14085915 | A/G |
| 3 | 14100048 | G/A |
| 3 | 14194325 | C/G |
| 3 | 14214494 | G/C |
| 3 | 14228517 | A/G |
| 3 | 14239805 | C/T |
| 3 | 14253345 | A/G |
| 3 | 14284048 | T/C |
| 3 | 14334410 | T/C |
| 3 | 14341910 | C/G |
| 3 | 14398850 | G/A |
| 3 | 14416190 | G/A |
| 3 | 14420604 | T/G |
| 3 | 14434650 | T/A |
| 3 | 14449403 | G/A |
| 3 | 14462605 | C/T |
| 3 | 14477480 | T/G |
| 3 | 14507077 | C/G |
| 3 | 14540282 | T/C |
| 3 | 14541571 | A/G |
| 3 | 14558597 | T/G |
| 3 | 14579835 | G/A |
| 3 | 14585777 | C/T |
| 3 | 14592106 | A/G |
| 3 | 14614295 | T/C |
| 3 | 14642046 | G/A |
| 3 | 14656935 | T/A |
| 3 | 14685964 | A/C |
| 3 | 14698972 | A/G |
| 3 | 14705043 | G/A |
| 3 | 14744484 | T/C |
| 3 | 14746943 | C/T |
| 3 | 14764085 | G/A |
| 3 | 14791295 | C/T |
| 3 | 14831165 | G/A |
| 3 | 14862485 | T/G |
| 3 | 14875901 | T/G |
| 3 | 14881406 | G/A |
| 3 | 14885660 | T/C |
| 3 | 14921975 | C/T |
| 3 | 14939146 | C/T |
| 3 | 14961479 | C/T |
| 3 | 14963665 | G/A |
| 3 | 14971134 | G/A |
| 3 | 15007201 | A/G |
| 3 | 15032546 | T/C |
| 3 | 15056742 | G/A |
| 3 | 15087550 | G/A |
| 3 | 15088678 | A/G |
| 3 | 15095886 | C/G |
| 3 | 15123145 | C/A |
| 3 | 15157248 | G/T |
| 3 | 15172495 | A/T |
| 3 | 15207399 | G/T |
| 3 | 15210624 | A/G |
| 3 | 15250522 | A/C |
| 3 | 15271341 | G/T |
| 3 | 15306354 | C/A |
| 3 | 15308063 | G/C |
| 3 | 15388404 | T/C |
| 3 | 15411826 | T/A |
| 3 | 15445295 | A/G |
| 3 | 15450348 | G/T |
| 3 | 15529053 | C/T |
| 3 | 15581348 | T/C |
| 3 | 15583328 | T/C |
| 3 | 15608440 | T/C |
| 3 | 15662792 | T/C |
| 3 | 15677639 | G/A |
| 3 | 15777408 | T/C |
| 3 | 15798681 | C/T |
| 3 | 15802779 | C/T |
| 3 | 15824388 | G/A |
| 3 | 15835521 | C/T |
| 3 | 15867102 | A/C |
| 3 | 15873005 | C/T |
| 3 | 15904106 | C/A |
| 3 | 15926024 | C/A |
| 3 | 15942467 | A/G |
| 3 | 15982148 | G/A |
| 3 | 16006518 | C/T |
| 3 | 16042965 | C/T |
| 3 | 16047764 | G/T |
| 3 | 16050082 | A/G |
| 3 | 16099433 | G/T |
| 3 | 16126946 | A/G |
| 3 | 16189749 | C/G |
| 3 | 16206636 | G/C |
| 3 | 16235237 | A/G |
| 3 | 16250585 | G/A |
| 3 | 16275332 | T/C |
| 3 | 16298024 | G/C |
| 3 | 16317932 | C/T |
| 3 | 16333022 | A/G |
| 3 | 16349661 | A/C |
| 3 | 16358459 | T/A |
| 3 | 16394363 | A/T |
| 3 | 16419236 | A/T |
| 3 | 16436795 | G/A |
| 3 | 16463218 | G/A |
| 3 | 16494944 | T/C |
| 3 | 16498523 | G/A |
| 3 | 16508101 | A/G |
| 3 | 16537370 | A/G |
| 3 | 16576281 | A/G |
| 3 | 16588112 | T/C |
| 3 | 16600302 | T/G |
| 3 | 16636808 | A/C |
| 3 | 16644212 | C/T |
| 3 | 16654143 | A/G |
| 3 | 16676129 | T/C |
| 3 | 16700058 | C/T |
| 3 | 16716797 | T/C |
| 3 | 16725538 | A/G |
| 3 | 16747772 | T/C |
| 3 | 16783192 | C/A |
| 3 | 16809871 | A/G |
| 3 | 16830058 | C/T |
| 3 | 16834134 | G/A |
| 3 | 16868992 | T/C |
| 3 | 16898157 | G/A |
| 3 | 16912557 | A/G |
| 3 | 16936652 | A/G |
| 3 | 16938700 | C/G |
| 3 | 16966606 | A/G |
| 3 | 16994910 | A/C |
| 3 | 17001356 | C/A |
| 3 | 17011201 | C/G |
| 3 | 17033715 | C/T |
| 3 | 17040305 | C/T |
| 3 | 17045081 | G/A |
| 3 | 17083623 | C/T |
| 3 | 17084786 | C/T |
| 3 | 17100837 | G/A |
| 3 | 17132827 | G/A |
| 3 | 17142648 | T/C |
| 3 | 17161667 | C/T |
| 3 | 17204241 | A/G |
| 3 | 17211336 | G/A |
| 3 | 17222386 | C/G |
| 3 | 17260110 | C/T |
| 3 | 17263018 | T/G |
| 3 | 17280834 | C/G |
| 3 | 17317227 | C/A |
| 3 | 17323113 | T/G |
| 3 | 17364883 | C/A |
| 3 | 17376025 | G/T |
| 3 | 17382869 | C/T |
| 3 | 17401240 | C/T |
| 3 | 17415947 | A/T |
| 3 | 17443944 | A/T |
| 3 | 17454922 | T/C |
| 3 | 17484175 | T/C |
| 3 | 17487381 | G/C |
| 3 | 17498464 | G/T |
| 3 | 17513904 | C/G |
| 3 | 17543152 | G/T |
| 3 | 17544183 | T/C |
| 3 | 17550171 | C/A |
| 3 | 17552685 | C/A |
| 3 | 17591194 | C/G |
| 3 | 17608025 | C/T |
| 3 | 17622838 | C/T |
| 3 | 17633478 | A/G |
| 3 | 17643324 | A/G |
| 3 | 17687129 | G/A |
| 3 | 17701529 | T/C |
| 3 | 17703305 | A/C |
| 3 | 17743016 | G/A |
| 3 | 17758731 | C/T |
| 3 | 17763171 | A/G |
| 3 | 17769010 | C/A |
| 3 | 17804162 | T/A |
| 3 | 17823156 | G/T |
| 3 | 17851386 | T/C |
| 3 | 17893221 | C/A |
| 3 | 17899127 | G/T |
| 3 | 17910094 | C/G |
| 3 | 17948934 | A/C |
| 3 | 17970894 | C/T |
| 3 | 17995122 | C/T |
| 3 | 18006624 | T/A |
| 3 | 18026173 | G/C |
| 3 | 18031487 | A/C |
| 3 | 18064970 | T/C |
| 3 | 18194674 | A/G |
| 3 | 18123875 | T/C |
| 3 | 18157188 | C/T |
| 3 | 18163198 | T/A |
| 3 | 18197013 | C/T |
| 3 | 18210280 | G/A |
| 3 | 18244669 | G/C |
| 3 | 18266196 | G/T |
| 3 | 18283809 | G/A |
| 3 | 18304254 | A/G |
| 3 | 18306602 | A/G |
| 3 | 18306603 | G/A |
| 3 | 18306604 | G/A |
| 3 | 18306605 | G/A |
| 3 | 18360594 | A/G |
| 3 | 18364910 | A/T |
| 3 | 18394161 | C/T |
| 3 | 18408932 | A/C |
| 3 | 18429188 | A/G |
| 3 | 18476368 | C/T |
| 3 | 18482969 | G/A |
| 3 | 18510308 | T/C |
| 3 | 18529435 | T/C |
| 3 | 18545771 | T/C |
| 3 | 18578883 | G/A |
| 3 | 18589422 | A/C |
| 3 | 18607667 | G/A |
| 3 | 18627192 | T/C |
| 3 | 18643527 | T/C |
| 3 | 18649180 | G/A |
| 3 | 18661268 | G/A |
| 3 | 18688724 | A/G |
| 3 | 18701067 | C/G |
| 3 | 18758734 | G/A |
| 3 | 18785963 | T/A |
| 3 | 18812028 | G/A |
| 3 | 18848631 | C/T |
| 3 | 18854273 | A/G |
| 3 | 18871468 | T/C |
| 3 | 18923935 | A/G |
| 3 | 18923936 | G/A |
| 3 | 18923937 | G/A |
| 3 | 18923938 | G/A |
| 3 | 18966569 | T/A |
| 3 | 18999348 | C/T |
| 3 | 19013545 | T/C |
| 3 | 19031801 | T/C |
| 3 | 19060824 | C/T |
| 3 | 19068781 | T/C |
| 3 | 19085150 | G/T |
| 3 | 19092989 | T/C |
| 3 | 19110632 | G/A |
| 3 | 19126203 | C/T |
| 3 | 19147834 | G/A |
| 3 | 19153996 | T/G |
| 3 | 19176509 | G/A |
| 3 | 19216167 | G/A |
| 3 | 19220686 | T/A |
| 3 | 19237550 | G/A |
| 3 | 19240860 | A/G |
| 3 | 19278928 | T/C |
| 3 | 19287084 | G/A |
| 3 | 19292363 | T/C |
| 3 | 19376716 | A/C |
| 3 | 19384861 | C/T |
| 3 | 19389124 | C/T |
| 3 | 19424143 | C/A |
| 3 | 19463601 | G/T |
| 3 | 19469682 | C/T |
| 3 | 19484009 | G/A |
| 3 | 19500053 | G/A |
| 3 | 19523436 | T/C |
| 3 | 19530106 | A/C |
| 3 | 19588805 | T/C |
| 3 | 19592827 | C/T |
| 3 | 19619058 | G/A |
| 3 | 19620437 | C/T |
| 3 | 19633527 | G/A |
| 3 | 19663335 | T/C |
| 3 | 19665378 | C/A |
| 3 | 19697444 | T/C |
| 3 | 19712907 | A/C |
| 3 | 19724079 | G/A |
| 3 | 19761685 | C/T |
| 3 | 19789496 | T/C |
| 3 | 19802734 | A/C |
| 3 | 19805458 | A/G |
| 3 | 19839195 | G/A |
| 3 | 19847109 | G/A |
| 3 | 19864043 | G/A |
| 3 | 19865241 | C/A |
| 3 | 19897620 | C/T |
| 3 | 19899522 | T/A |
| 3 | 19941707 | G/A |
| 3 | 19953951 | C/A |
| 3 | 19963898 | C/T |
| 3 | 20007545 | A/T |
| 3 | 20029761 | G/A |
| 3 | 20060824 | C/T |
| 3 | 20047039 | C/T |
| 3 | 20097682 | G/A |
| 3 | 20126765 | A/G |
| 3 | 20138005 | C/T |
| 3 | 20148197 | C/T |
| 3 | 20193519 | C/A |
| 3 | 20222897 | A/G |
| 3 | 20277835 | G/A |
| 3 | 20288772 | G/A |
| 3 | 20307892 | G/A |
| 3 | 20315944 | C/T |
| 3 | 20318447 | C/T |
| 3 | 20353917 | C/T |
| 3 | 20367170 | C/T |
| 3 | 20424469 | A/G |
| 3 | 20440032 | C/A |
| 3 | 20460353 | A/G |
| 3 | 20475597 | C/A |
| 3 | 20500407 | A/G |
| 3 | 20526759 | C/T |
| 3 | 20567378 | G/A |
| 3 | 20619814 | C/T |
| 3 | 20630135 | C/T |
| 3 | 20662209 | C/T |
| 3 | 20685410 | C/A |
| 3 | 20705860 | A/G |
| 3 | 20733562 | C/T |
| 3 | 20739253 | C/G |
| 3 | 20797492 | G/A |
| 3 | 20826917 | G/A |
| 3 | 20847264 | C/T |
| 3 | 20868451 | A/G |
| 3 | 20885928 | A/G |
| 3 | 20905868 | C/T |
| 3 | 20928489 | C/T |
| 3 | 20940535 | G/T |
| 3 | 20976406 | C/T |
| 3 | 20996473 | C/T |
| 3 | 21023670 | C/G |
| 3 | 21041320 | C/G |
| 3 | 21061906 | A/G |
| 3 | 21093011 | C/T |
| 3 | 21096852 | C/A |
| 3 | 21130531 | C/T |
| 3 | 21175627 | G/T |
| 3 | 21199409 | C/T |
| 3 | 21216419 | C/T |
| 3 | 21238216 | C/T |
| 3 | 21261633 | A/G |
| 3 | 21289274 | C/T |
| 3 | 21307560 | T/C |
| 3 | 21326531 | C/T |
| 3 | 21338958 | C/T |
| 3 | 21355674 | G/T |
| 3 | 21362936 | C/A |
| 3 | 21426179 | T/C |
| 3 | 21427937 | G/A |
| 3 | 21467450 | G/A |
| 3 | 21487627 | C/T |
| 3 | 21534854 | C/T |
| 3 | 21595361 | G/A |
| 3 | 21597498 | G/A |
| 3 | 21615016 | C/T |
| 3 | 21634908 | G/A |
| 3 | 21649776 | A/G |
| 3 | 21662429 | T/C |
| 3 | 21685418 | T/C |
| 3 | 21715708 | G/A |
| 3 | 21760734 | C/T |
| 3 | 21762071 | T/A |
| 3 | 21785217 | G/A |
| 3 | 21809791 | A/G |
| 3 | 21825204 | C/T |
| 3 | 21856413 | C/A |
| 3 | 21962075 | G/A |
| 3 | 21999832 | C/A |
| 3 | 22036933 | C/T |
| 3 | 22044938 | A/G |
| 3 | 22058769 | C/T |
| 3 | 22074751 | C/T |
| 3 | 22128464 | G/A |
| 3 | 22153259 | C/A |
| 3 | 22161265 | G/A |
| 3 | 22207811 | G/A |
| 3 | 22260791 | T/C |
| 3 | 22320050 | G/T |
| 3 | 22365895 | C/A |
| 3 | 22389840 | G/A |
| 3 | 22421423 | C/A |
| 3 | 22455124 | G/A |
| 3 | 22490616 | C/T |
| 3 | 22523607 | A/G |
| 3 | 22544031 | T/C |
| 3 | 22557564 | T/C |
| 3 | 22567208 | C/T |
| 3 | 22582741 | C/T |
| 3 | 22590585 | A/G |
| 3 | 22604844 | C/T |
| 3 | 22626822 | G/A |
| 3 | 22631391 | G/A |
| 3 | 22653292 | A/G |
| 3 | 22698785 | C/T |
| 3 | 22725674 | A/G |
| 3 | 22736133 | G/A |
| 3 | 22749233 | G/A |
| 3 | 22775682 | C/G |
| 3 | 22806521 | C/T |
| 3 | 22837748 | C/T |
| 3 | 22859593 | G/A |
| 3 | 22867495 | G/C |
| 3 | 22888307 | T/C |
| 3 | 22891799 | C/T |
| 3 | 22942540 | G/C |
| 3 | 22946027 | T/C |
| 3 | 22953293 | T/A |
| 3 | 22959673 | G/A |
| 3 | 22984546 | G/C |
| 3 | 22999938 | T/C |
| 3 | 23024453 | C/A |
| 3 | 23042226 | C/T |
| 3 | 23069468 | G/A |
| 3 | 23094036 | A/G |
| 3 | 23165102 | A/C |
| 3 | 23197534 | G/T |
| 3 | 23222562 | T/A |
| 3 | 23235926 | C/T |
| 3 | 23268032 | A/C |
| 3 | 23272276 | C/T |
| 3 | 23285593 | T/C |
| 3 | 23303108 | C/T |
| 3 | 23314860 | G/A |
| 3 | 23356583 | C/T |
| 3 | 23362273 | C/T |
| 3 | 23393865 | A/C |
| 3 | 23410566 | G/A |
| 3 | 23416517 | G/A |
| 3 | 23436255 | G/A |
| 3 | 23468365 | G/A |
| 3 | 23480840 | A/G |
| 3 | 23548360 | G/A |
| 3 | 23549893 | T/G |
| 3 | 23553761 | G/A |
| 3 | 23625373 | G/A |
| 3 | 23644869 | A/G |
| 3 | 23680498 | A/G |
| 3 | 23719395 | C/T |
| 3 | 23762994 | C/T |
| 3 | 23855935 | T/C |
| 3 | 23870665 | G/C |
| 3 | 23918988 | C/A |
| 3 | 23944490 | A/C |
| 3 | 23978687 | C/T |
| 3 | 23996577 | C/A |
| 3 | 24054506 | C/T |
| 3 | 24071479 | T/C |
| 3 | 24099791 | C/T |
| 3 | 24104563 | A/G |
| 3 | 24133483 | T/C |
| 3 | 24141445 | G/A |
| 3 | 24151254 | G/A |
| 3 | 24171076 | C/T |
| 3 | 24229662 | C/T |
| 3 | 24265467 | T/C |
| 3 | 24278665 | C/T |
| 3 | 24300067 | T/G |
| 3 | 24325188 | T/C |
| 3 | 24348692 | G/A |
| 3 | 24349712 | A/G |
| 3 | 24368176 | G/A |
| 3 | 24373705 | G/T |
| 3 | 24416864 | C/T |
| 3 | 24434571 | G/A |
| 3 | 24447543 | G/A |
| 3 | 24498727 | T/C |
| 3 | 24502799 | C/T |
| 3 | 24533152 | T/G |
| 3 | 24534603 | G/A |
| 3 | 24563382 | A/G |
| 3 | 24575676 | C/T |
| 3 | 24623350 | A/T |
| 3 | 24629689 | T/C |
| 3 | 24656747 | C/A |
| 3 | 24670907 | C/T |
| 3 | 24698373 | G/A |
| 3 | 24715908 | T/C |
| 3 | 24724398 | T/C |
| 3 | 24764025 | C/T |
| 3 | 24784884 | A/G |
| 3 | 24806818 | C/T |
| 3 | 24831545 | G/T |
| 3 | 24865743 | T/C |
| 3 | 24870770 | T/C |
| 3 | 24906368 | G/A |
| 3 | 24954932 | G/T |
| 3 | 24956941 | C/T |
| 3 | 24963915 | G/A |
| 3 | 24974187 | T/G |
| 3 | 24992575 | T/C |
| 3 | 25007564 | C/T |
| 3 | 25028475 | T/C |
| 3 | 25051931 | G/A |
| 3 | 25079486 | A/T |
| 3 | 25119864 | A/C |
| 3 | 25138195 | T/A |
| 3 | 25141569 | C/T |
| 3 | 25158655 | T/G |
| 3 | 25183315 | A/G |
| 3 | 25206188 | G/A |
| 3 | 25237883 | T/C |
| 3 | 25242577 | T/C |
| 3 | 25271962 | T/A |
| 3 | 25287746 | C/T |
| 3 | 25310193 | C/T |
| 3 | 25322432 | C/T |
| 3 | 25375066 | C/T |
| 3 | 25415745 | G/A |
| 3 | 25417376 | C/T |
| 3 | 25447014 | C/A |
| 3 | 25455748 | C/T |
| 3 | 25523881 | C/T |
| 3 | 25526474 | C/T |
| 3 | 25552860 | T/C |
| 3 | 25566265 | C/T |
| 3 | 25594854 | T/C |
| 3 | 25620622 | T/C |
| 3 | 25674108 | A/G |
| 3 | 25691531 | C/G |
| 3 | 25732113 | C/A |
| 3 | 25763430 | G/A |
| 3 | 25778526 | T/A |
| 3 | 25804476 | C/T |
| 3 | 25851261 | T/C |
| 3 | 25870066 | C/T |
| 3 | 25893556 | A/G |
| 3 | 25916292 | G/A |
| 3 | 25934894 | C/T |
| 3 | 25957775 | C/A |
| 3 | 25961161 | A/G |
| 3 | 26001703 | C/T |
| 3 | 26009180 | C/T |
| 3 | 26022814 | G/T |
| 3 | 26035752 | T/C |
| 3 | 26066809 | C/T |
| 3 | 26084548 | A/G |
| 3 | 26137023 | C/T |
| 3 | 26165797 | A/C |
| 3 | 26202280 | C/G |
| 3 | 26222360 | C/T |
| 3 | 26256397 | G/T |
| 3 | 26292559 | T/C |
| 3 | 26322462 | A/G |
| 3 | 26381063 | G/A |
| 3 | 26388524 | C/T |
| 3 | 26401932 | C/T |
| 3 | 26462885 | G/A |
| 3 | 26512682 | T/G |
| 3 | 26520092 | T/G |
| 3 | 26573060 | A/T |
| 3 | 26578970 | G/A |
| 3 | 26587276 | T/C |
| 3 | 26599223 | G/A |
| 3 | 26610171 | G/C |
| 3 | 26638800 | T/C |
| 3 | 26645075 | G/A |
| 3 | 26653472 | G/T |
| 3 | 26691602 | T/C |
| 3 | 26698155 | T/C |
| 3 | 26709667 | C/T |
| 3 | 26730486 | T/A |
| 3 | 26746407 | A/C |
| 3 | 26764614 | T/G |
| 3 | 26790324 | C/T |
| 3 | 26832529 | T/C |
| 3 | 26844046 | G/A |
| 3 | 26852441 | G/A |
| 3 | 26880575 | C/A |
| 3 | 26894579 | C/T |
| 3 | 26921651 | C/T |
| 3 | 26924200 | G/A |
| 3 | 26974516 | G/A |
| 3 | 27002439 | A/T |
| 3 | 27020284 | C/A |
| 3 | 27051936 | C/T |
| 3 | 27074390 | G/A |
| 3 | 27074391 | C/T |
| 3 | 27289132 | G/A |
| 3 | 27328459 | C/T |
| 3 | 27341999 | C/T |
| 3 | 27395633 | G/T |
| 3 | 27413925 | C/T |
| 3 | 27453505 | C/T |
| 3 | 27455400 | G/A |
| 3 | 27505861 | C/T |
| 3 | 27524016 | G/A |
| 3 | 27529130 | G/A |
| 3 | 27560939 | C/T |
| 3 | 27569400 | C/T |
| 3 | 27582477 | G/A |
| 3 | 27588142 | C/T |
| 3 | 27612750 | A/T |
| 3 | 27633486 | A/T |
| 3 | 27654103 | C/T |
| 3 | 27676162 | T/C |
| 3 | 27698961 | G/A |
| 3 | 27713075 | C/T |
| 3 | 27740838 | T/C |
| 3 | 27750492 | C/T |
| 3 | 27779100 | G/A |
| 3 | 27798272 | C/T |
| 3 | 27861949 | A/T |
| 3 | 27868334 | G/A |
| 3 | 27895101 | T/C |
| 3 | 27921755 | G/A |
| 3 | 27937576 | G/A |
| 3 | 28007534 | G/A |
| 3 | 28028538 | C/T |
| 3 | 28065228 | A/G |
| 3 | 28116699 | T/C |
| 3 | 28149000 | G/A |
| 3 | 28157509 | A/G |
| 3 | 28176429 | A/C |
| 3 | 28183975 | T/A |
| 3 | 28209866 | C/T |
| 3 | 28234557 | G/A |
| 3 | 28301026 | C/T |
| 3 | 28316500 | G/A |
| 3 | 28332881 | T/C |
| 3 | 28371536 | C/T |
| 3 | 28405463 | G/A |
| 3 | 28416474 | G/A |
| 3 | 28431769 | C/T |
| 3 | 28453974 | C/T |
| 3 | 28471538 | C/G |
| 3 | 28515519 | T/C |
| 3 | 28539525 | C/T |
| 3 | 28565934 | A/T |
| 3 | 28598913 | G/A |
| 3 | 28620744 | C/A |
| 3 | 28636688 | C/T |
| 3 | 28651866 | A/G |
| 3 | 28660964 | G/C |
| 3 | 28695678 | G/A |
| 3 | 28698650 | G/C |
| 3 | 28738517 | C/T |
| 3 | 28743236 | C/T |
| 3 | 28769427 | G/A |
| 3 | 28821679 | A/G |
| 3 | 28823767 | C/A |
| 3 | 28842310 | G/A |
| 3 | 28869254 | C/T |
| 3 | 28899424 | C/T |
| 3 | 28912852 | A/G |
| 3 | 28925798 | T/C |
| 3 | 29003683 | A/G |
| 3 | 29006525 | A/G |
| 3 | 29025765 | C/T |
| 3 | 29030911 | T/C |
| 3 | 29040853 | G/A |
| 3 | 29042950 | C/A |
| 3 | 29073099 | T/A |
| 3 | 29077478 | C/T |
| 3 | 29116040 | T/C |
| 3 | 29157481 | C/T |
| 3 | 29182634 | A/G |
| 3 | 29211844 | C/T |
| 3 | 29231775 | G/C |
| 3 | 29237745 | T/C |
| 3 | 29277492 | G/A |
| 3 | 29303469 | A/G |
| 3 | 29304841 | G/C |
| 3 | 29324672 | G/A |
| 3 | 29363193 | G/A |
| 3 | 29372692 | G/T |
| 3 | 29415779 | C/T |
| 3 | 29435845 | G/A |
| 3 | 29488479 | C/T |
| 3 | 29501299 | G/A |
| 3 | 29511624 | G/A |
| 3 | 29533413 | G/A |
| 3 | 29540827 | G/C |
| 3 | 29550882 | C/T |
| 3 | 29572707 | G/A |
| 3 | 29582328 | C/T |
| 3 | 29591835 | G/A |
| 3 | 29619011 | G/A |
| 3 | 29669567 | A/G |
| 3 | 29697254 | C/T |
| 3 | 29728247 | C/T |
| 3 | 29740666 | G/A |
| 3 | 29768861 | A/G |
| 3 | 29781371 | C/T |
| 3 | 29795944 | G/A |
| 3 | 29829475 | G/A |
| 3 | 29843440 | A/T |
| 3 | 29877000 | T/C |
| 3 | 29881333 | C/T |
| 3 | 29892153 | G/A |
| 3 | 29944612 | G/A |
| 3 | 29953718 | G/A |
| 3 | 29980133 | A/G |
| 3 | 29991416 | A/G |
| 3 | 30003775 | C/T |
| 3 | 30019467 | T/A |
| 3 | 30051813 | G/A |
| 3 | 30065742 | G/A |
| 3 | 30092950 | C/T |
| 3 | 30132208 | C/T |
| 3 | 30149412 | G/C |
| 3 | 30162396 | G/A |
| 3 | 30166244 | C/A |
| 3 | 30190661 | C/T |
| 3 | 30216088 | C/G |
| 3 | 30264393 | C/T |
| 3 | 30287236 | C/A |
| 3 | 30305860 | T/C |
| 3 | 30346804 | G/T |
| 3 | 30366124 | A/G |
| 3 | 30398083 | C/T |
| 3 | 30429194 | G/A |
| 3 | 30453055 | G/A |
| 3 | 30456872 | G/A |
| 3 | 30508174 | G/A |
| 3 | 30524266 | A/T |
| 3 | 30554251 | C/A |
| 3 | 30593955 | C/T |
| 3 | 30608576 | G/A |
| 3 | 30612007 | C/A |
| 3 | 30688103 | C/T |
| 3 | 30696548 | C/A |
| 3 | 30721630 | C/T |
| 3 | 30728565 | C/T |
| 3 | 30802526 | A/C |
| 3 | 30804287 | G/T |
| 3 | 30812486 | T/C |
| 3 | 30848465 | G/A |
| 3 | 30888603 | G/A |
| 3 | 30891699 | C/T |
| 3 | 30901466 | G/A |
| 3 | 30929734 | G/A |
| 3 | 30938532 | A/T |
| 3 | 30947565 | C/T |
| 3 | 30965302 | G/A |
| 3 | 31007411 | A/G |
| 3 | 31040633 | G/A |
| 3 | 31087713 | G/A |
| 3 | 31122508 | C/G |
| 3 | 31123800 | C/A |
| 3 | 31168105 | A/G |
| 3 | 31170512 | T/C |
| 3 | 31211395 | C/T |
| 3 | 31244090 | C/T |
| 3 | 31263048 | T/C |
| 3 | 31296224 | G/A |
| 3 | 31318717 | G/A |
| 3 | 31331305 | C/T |
| 3 | 31348299 | T/C |
| 3 | 31375519 | A/G |
| 3 | 31415781 | A/G |
| 3 | 31443378 | C/T |
| 3 | 31586262 | T/C |
| 3 | 31611961 | C/T |
| 3 | 31620548 | G/A |
| 3 | 31635142 | A/G |
| 3 | 31652702 | C/T |
| 3 | 31687571 | T/C |
| 3 | 31728714 | G/A |
| 3 | 31732421 | C/A |
| 3 | 31762213 | C/T |
| 3 | 31825858 | T/C |
| 3 | 31878114 | C/T |
| 3 | 31998557 | T/C |
| 3 | 32027645 | C/T |
| 3 | 32042035 | G/A |
| 3 | 32076989 | T/C |
| 3 | 32091950 | C/T |
| 3 | 32100949 | G/A |
| 3 | 32116662 | G/A |
| 3 | 32156480 | T/C |
| 3 | 32163781 | G/A |
| 3 | 32185224 | C/T |
| 3 | 32223486 | G/A |
| 3 | 32237690 | C/T |
| 3 | 32270707 | T/G |
| 3 | 32323276 | A/C |
| 3 | 32342863 | T/C |
| 3 | 32416851 | G/A |
| 3 | 32449382 | G/A |
| 3 | 32477951 | T/C |
| 3 | 32510952 | C/T |
| 3 | 32540877 | A/C |
| 3 | 32544565 | C/T |
| 3 | 32551437 | C/T |
| 3 | 32588349 | T/G |
| 3 | 32623778 | G/A |
| 3 | 32650533 | G/T |
| 3 | 32720668 | C/T |
| 3 | 32728230 | G/A |
| 3 | 32745098 | G/A |
| 3 | 32815038 | C/T |
| 3 | 32829355 | T/C |
| 3 | 32873560 | C/T |
| 3 | 32895772 | G/A |
| 3 | 32989225 | G/A |
| 3 | 33009740 | A/G |
| 3 | 33164808 | G/T |
| 3 | 33189226 | C/T |
| 3 | 33198563 | C/G |
| 3 | 33238459 | C/T |
| 3 | 33240068 | C/T |
| 3 | 33241946 | C/T |
| 3 | 33295820 | C/T |
| 3 | 33316783 | T/C |
| 3 | 33413987 | G/A |
| 3 | 33422599 | C/T |
| 3 | 33516701 | T/G |
| 3 | 33556203 | C/T |
| 3 | 33558356 | T/C |
| 3 | 33582784 | C/T |
| 3 | 33594304 | C/G |
| 3 | 33618816 | G/A |
| 3 | 33641755 | G/A |
| 3 | 33668511 | G/A |
| 3 | 33707907 | G/A |
| 3 | 33744900 | T/A |
| 3 | 33775876 | C/T |
| 3 | 33807652 | G/A |
| 3 | 33829887 | T/C |
| 3 | 33849881 | G/T |
| 3 | 33949710 | C/G |
| 3 | 33957375 | C/A |
| 3 | 33967233 | C/T |
| 3 | 33968295 | A/G |
| 3 | 34034160 | C/T |
| 3 | 34046333 | T/C |
| 3 | 34061319 | C/T |
| 3 | 34066892 | C/T |
| 3 | 34085771 | C/G |
| 3 | 34126582 | A/G |
| 3 | 34132429 | G/C |
| 3 | 34146453 | C/T |
| 3 | 34233532 | G/A |
| 3 | 34240545 | C/G |
| 3 | 34308330 | G/A |
| 3 | 34337883 | C/T |
| 3 | 34343346 | T/C |
| 3 | 34375924 | C/T |
| 3 | 34387092 | G/A |
| 3 | 34408531 | A/G |
| 3 | 34412655 | C/T |
| 3 | 34467226 | T/C |
| 3 | 34485387 | C/T |
| 3 | 34508110 | T/C |
| 3 | 34517957 | T/G |
| 3 | 34530673 | C/T |
| 3 | 34541058 | C/T |
| 3 | 34587460 | T/C |
| 3 | 34588472 | T/C |
| 3 | 34612563 | G/A |
| 3 | 34621793 | A/G |
| 3 | 34644613 | T/G |
| 3 | 34650212 | C/T |
| 3 | 34651591 | G/A |
| 3 | 34694383 | A/G |
| 3 | 34713335 | A/C |
| 3 | 34723871 | C/T |
| 3 | 34747389 | C/T |
| 3 | 34785227 | A/G |
| 3 | 34795129 | G/C |
| 3 | 34819535 | C/T |
| 3 | 34832403 | A/G |
| 3 | 34880401 | G/C |
| 3 | 34885750 | C/T |
| 3 | 34890098 | C/T |
| 3 | 34932075 | G/A |
| 3 | 34934464 | C/T |
| 3 | 34960833 | C/T |
| 3 | 34961939 | C/T |
| 3 | 34989283 | T/C |
| 3 | 34996729 | A/G |
| 3 | 35013463 | A/G |
| 3 | 35032285 | T/C |
| 3 | 35045448 | G/A |
| 3 | 35067226 | C/T |
| 3 | 35070519 | A/C |
| 3 | 35084430 | C/T |
| 3 | 35102023 | A/G |
| 3 | 35107683 | A/G |
| 3 | 35159685 | T/C |
| 3 | 35171891 | T/C |
| 3 | 35175151 | A/T |
| 3 | 35197981 | C/A |
| 3 | 35230797 | A/G |
| 3 | 35262046 | A/G |
| 3 | 35319678 | C/T |
| 3 | 35352939 | C/G |
| 3 | 35388400 | G/A |
| 3 | 35395325 | G/A |
| 3 | 35427958 | T/C |
| 3 | 35448619 | A/T |
| 3 | 35454656 | G/A |
| 3 | 35475006 | T/C |
| 3 | 35488222 | C/T |
| 3 | 35498049 | A/G |
| 3 | 35502678 | A/G |
| 3 | 35532668 | G/A |
| 3 | 35553624 | C/A |
| 3 | 35559451 | A/C |
| 3 | 35585801 | A/G |
| 3 | 35616147 | G/T |
| 3 | 35668525 | C/T |
| 3 | 35670962 | T/G |
| 3 | 35681847 | G/C |
| 3 | 35718212 | T/G |
| 3 | 35730492 | A/G |
| 3 | 35754631 | C/G |
| 3 | 35811708 | C/G |
| 3 | 35820648 | A/G |
| 3 | 35827415 | G/A |
| 3 | 35856291 | C/A |
| 3 | 35866547 | T/A |
| 3 | 35897261 | G/A |
| 3 | 35916528 | C/A |
| 3 | 35917622 | G/A |
| 3 | 35950734 | C/T |
| 3 | 35965143 | C/T |
| 3 | 35978203 | A/T |
| 3 | 35983727 | G/A |
| 3 | 36002567 | C/T |
| 3 | 36048192 | A/G |
| 3 | 36084363 | A/C |
| 3 | 36088907 | C/T |
| 3 | 36102529 | G/A |
| 3 | 36122061 | T/C |
| 3 | 36161728 | C/G |
| 3 | 36173682 | C/T |
| 3 | 36188960 | A/G |
| 3 | 36227457 | T/C |
| 3 | 36229706 | C/A |
| 3 | 36249033 | A/G |
| 3 | 36285931 | G/A |
| 3 | 36313529 | G/A |
| 3 | 36416787 | T/C |
| 3 | 36435997 | G/A |
| 3 | 36479870 | G/C |
| 3 | 36500705 | G/A |
| 3 | 36508920 | T/C |
| 3 | 36526610 | A/G |
| 3 | 36529519 | T/C |
| 3 | 36559294 | A/G |
| 3 | 36584913 | A/G |
| 3 | 36587102 | T/A |
| 3 | 36612557 | C/T |
| 3 | 36631294 | T/G |
| 3 | 36640123 | C/T |
| 3 | 36683453 | T/C |
| 3 | 36689458 | A/G |
| 3 | 36692642 | T/C |
| 3 | 36698043 | G/T |
| 3 | 36730031 | G/C |
| 3 | 36751156 | C/A |
| 3 | 36779034 | C/G |
| 3 | 36801950 | A/T |
| 3 | 36803120 | T/C |
| 3 | 36814367 | T/C |
| 3 | 36831999 | G/C |
| 3 | 36840753 | C/T |
| 3 | 36860575 | T/C |
| 3 | 36877533 | A/G |
| 3 | 36931915 | G/T |
| 3 | 36943948 | G/A |
| 3 | 37069936 | C/T |
| 3 | 37217332 | T/C |
| 3 | 37229046 | A/G |
| 3 | 37242507 | C/T |
| 3 | 37250231 | G/A |
| 3 | 37261938 | C/T |
| 3 | 37268792 | G/T |
| 3 | 37305775 | T/C |
| 3 | 37337418 | C/T |
| 3 | 37343616 | G/A |
| 3 | 37359980 | A/T |
| 3 | 37399407 | C/T |
| 3 | 37404368 | C/T |
| 3 | 37412204 | G/C |
| 3 | 37451589 | G/A |
| 3 | 37432280 | A/G |
| 3 | 37487612 | T/C |
| 3 | 37531205 | T/C |
| 3 | 37551967 | C/T |
| 3 | 37565033 | G/A |
| 3 | 37580495 | G/A |
| 3 | 37591255 | C/T |
| 3 | 37624594 | A/G |
| 3 | 37643059 | A/G |
| 3 | 37655213 | T/C |
| 3 | 37655214 | T/C |
| 3 | 37655215 | T/C |
| 3 | 37655216 | T/C |
| 3 | 37655217 | G/A |
| 3 | 37655218 | G/A |
| 3 | 37655219 | G/A |
| 3 | 37655220 | C/T |
| 3 | 37785748 | C/G |
| 3 | 37798081 | G/T |
| 3 | 37804583 | C/T |
| 3 | 37823927 | C/T |
| 3 | 37833636 | C/T |
| 3 | 37851730 | T/G |
| 3 | 37879909 | A/G |
| 3 | 37896983 | C/T |
| 3 | 37921244 | C/T |
| 3 | 37951618 | A/G |
| 3 | 37974631 | T/C |
| 3 | 38008352 | A/T |
| 3 | 38020259 | G/A |
| 3 | 38023440 | A/G |
| 3 | 38042379 | T/C |
| 3 | 38058238 | C/T |
| 3 | 38091164 | A/G |
| 3 | 38105715 | C/T |
| 3 | 38157222 | C/T |
| 3 | 38184367 | A/G |
| 3 | 38191474 | C/T |
| 3 | 38219586 | T/C |
| 3 | 38236554 | G/A |
| 3 | 38242159 | G/A |
| 3 | 38275244 | G/C |
| 3 | 38282074 | G/A |
| 3 | 38312219 | T/C |
| 3 | 38344355 | G/A |
| 3 | 38349656 | G/A |
| 3 | 38397128 | C/A |
| 3 | 38399191 | G/A |
| 3 | 38406947 | A/G |
| 3 | 38439402 | T/G |
| 3 | 38465225 | C/T |
| 3 | 38488850 | A/T |
| 3 | 38494285 | A/G |
| 3 | 38513950 | C/T |
| 3 | 38540675 | G/A |
| 3 | 38544403 | T/A |
| 3 | 38554777 | C/T |
| 3 | 38587992 | C/T |
| 3 | 38599874 | G/A |
| 3 | 38611807 | G/A |
| 3 | 38646963 | C/T |
| 3 | 38664802 | G/A |
| 3 | 38680139 | C/A |
| 3 | 38703670 | C/A |
| 3 | 38715996 | C/T |
| 3 | 38730759 | C/A |
| 3 | 38736743 | G/A |
| 3 | 38783991 | G/A |
| 3 | 38794512 | T/G |
| 3 | 38797029 | A/G |
| 3 | 38824488 | C/T |
| 3 | 38858622 | T/G |
| 3 | 38860101 | G/A |
| 3 | 38885395 | C/T |
| 3 | 38893899 | T/A |
| 3 | 38932675 | A/T |
| 3 | 38948928 | C/A |
| 3 | 38968576 | T/C |
| 3 | 38977466 | G/A |
| 3 | 38980513 | T/A |
| 3 | 39002250 | T/C |
| 3 | 39004419 | G/T |
| 3 | 39038047 | G/A |
| 3 | 39051239 | C/T |
| 3 | 39078081 | C/T |
| 3 | 39106632 | C/T |
| 3 | 39115612 | G/A |
| 3 | 39134468 | T/G |
| 3 | 39136822 | G/A |
| 3 | 39184178 | A/G |
| 3 | 39218579 | T/C |
| 3 | 39236231 | C/G |
| 3 | 39259467 | C/T |
| 3 | 39322201 | G/C |
| 3 | 39346211 | G/T |
| 3 | 39348682 | T/C |
| 3 | 39363671 | T/C |
| 3 | 39374184 | G/C |
| 3 | 39420424 | C/T |
| 3 | 39427384 | A/C |
| 3 | 39454970 | G/T |
| 3 | 39466172 | A/G |
| 3 | 39613776 | G/C |
| 3 | 39629914 | T/G |
| 3 | 39631071 | G/A |
| 3 | 39643545 | C/T |
| 3 | 39668116 | G/A |
| 3 | 39670640 | C/T |
| 3 | 39692549 | G/C |
| 3 | 39720130 | G/A |
| 3 | 39753997 | C/T |
| 3 | 39757221 | C/T |
| 3 | 39817417 | C/T |
| 3 | 39826274 | T/G |
| 3 | 39849356 | G/A |
| 3 | 39867529 | G/A |
| 3 | 39899513 | A/C |
| 3 | 39905651 | G/A |
| 3 | 39954690 | G/A |
| 3 | 39963112 | G/A |
| 3 | 39983078 | G/A |
| 3 | 40019425 | C/T |
| 3 | 40025522 | C/T |
| 3 | 40089343 | C/T |
| 3 | 40186041 | G/A |
| 3 | 40211381 | G/A |
| 3 | 40277489 | A/T |
| 3 | 40295918 | T/C |
| 3 | 40302994 | T/C |
| 3 | 40355230 | G/A |
| 3 | 40365532 | A/G |
| 3 | 40403074 | A/G |
| 3 | 40437442 | A/G |
| 3 | 40440231 | G/A |
| 3 | 40485312 | G/A |
| 3 | 40507183 | C/T |
| 3 | 40527185 | T/A |
| 3 | 40544433 | T/C |
| 3 | 40558459 | T/C |
| 3 | 40580849 | G/A |
| 3 | 40585231 | T/A |
| 3 | 40610484 | G/A |
| 3 | 40613959 | T/C |
| 3 | 40621717 | G/C |
| 3 | 40669876 | C/T |
| 3 | 40708551 | C/T |
| 3 | 40727843 | A/C |
| 3 | 40731510 | T/C |
| 3 | 40749788 | G/A |
| 3 | 40760799 | T/C |
| 3 | 40760800 | G/A |
| 3 | 40803948 | C/T |
| 3 | 40835641 | G/T |
| 3 | 40852401 | G/A |
| 3 | 40911212 | C/T |
| 3 | 40913558 | G/C |
| 3 | 40943693 | C/T |
| 3 | 40944766 | G/A |
| 3 | 40956991 | C/T |
| 3 | 40971468 | C/T |
| 3 | 41001896 | C/A |
| 3 | 41030618 | G/A |
| 3 | 41120672 | G/A |
| 3 | 41133764 | C/A |
| 3 | 41148531 | T/C |
| 3 | 41180701 | G/A |
| 3 | 41195730 | T/C |
| 3 | 41201327 | A/G |
| 3 | 41247502 | A/C |
| 3 | 41250681 | A/T |
| 3 | 41259121 | G/A |
| 3 | 41298048 | G/A |
| 3 | 41313889 | G/T |
| 3 | 41320786 | C/T |
| 3 | 41351918 | T/A |
| 3 | 41376641 | C/T |
| 3 | 41380772 | C/A |
| 3 | 41423942 | A/G |
| 3 | 41428925 | T/G |
| 3 | 41431444 | G/A |
| 3 | 41432531 | C/T |
| 3 | 41464327 | G/C |
| 3 | 41499461 | G/A |
| 3 | 41515233 | C/T |
| 3 | 41524917 | C/T |
| 3 | 41546300 | A/T |
| 3 | 41562115 | C/A |
| 3 | 41581506 | A/G |
| 3 | 41627103 | T/C |
| 3 | 41630645 | T/C |
| 3 | 41690110 | A/C |
| 3 | 41691348 | C/T |
| 3 | 41700465 | C/T |
| 3 | 41702047 | A/G |
| 3 | 41730676 | G/A |
| 3 | 41764705 | G/A |
| 3 | 41792117 | G/C |
| 3 | 41820809 | C/T |
| 3 | 41839697 | G/A |
| 3 | 41850108 | A/G |
| 3 | 41897424 | T/C |
| 3 | 41913815 | G/A |
| 3 | 41948747 | T/C |
| 3 | 41964042 | G/A |
| 3 | 41970570 | A/T |
| 3 | 41975012 | G/A |
| 3 | 42000191 | C/A |
| 3 | 42003430 | G/T |
| 3 | 42040591 | A/C |
| 3 | 42045797 | T/G |
| 3 | 42069217 | A/T |
| 3 | 42080407 | G/C |
| 3 | 42117202 | A/G |
| 3 | 42166575 | A/G |
| 3 | 42172240 | G/A |
| 3 | 42232083 | G/A |
| 3 | 42250502 | C/T |
| 3 | 42280175 | T/C |
| 3 | 42317967 | C/T |
| 3 | 42326338 | T/A |
| 3 | 42366914 | T/C |
| 3 | 42411845 | G/T |
| 3 | 42440566 | T/A |
| 3 | 42448384 | G/A |
| 3 | 42451035 | T/C |
| 3 | 42487386 | G/A |
| 3 | 42492147 | A/G |
| 3 | 42537756 | A/G |
| 3 | 42552062 | T/G |
| 3 | 42567730 | T/A |
| 3 | 42572867 | C/T |
| 3 | 42596269 | C/T |
| 3 | 42615689 | G/A |
| 3 | 42625519 | G/A |
| 3 | 42632208 | C/T |
| 3 | 42688911 | G/A |
| 3 | 42702347 | C/T |
| 3 | 42708492 | A/G |
| 3 | 42722352 | C/G |
| 3 | 42730950 | G/C |
| 3 | 42754056 | C/A |
| 3 | 42759455 | G/A |
| 3 | 42797943 | C/T |
| 3 | 42818562 | G/A |
| 3 | 42842767 | A/T |
| 3 | 42878319 | G/T |
| 3 | 42923041 | C/G |
| 3 | 42931177 | C/A |
| 3 | 42941132 | C/T |
| 3 | 42964928 | C/G |
| 3 | 42971945 | T/C |
| 3 | 42993696 | C/T |
| 3 | 42996147 | C/T |
| 3 | 43068978 | A/C |
| 3 | 43075960 | G/A |
| 3 | 43100034 | T/C |
| 3 | 43103732 | G/T |
| 3 | 43110312 | G/A |
| 3 | 43121105 | T/C |
| 3 | 43147341 | G/A |
| 3 | 43157233 | A/G |
| 3 | 43174666 | T/C |
| 3 | 43193959 | C/T |
| 3 | 43224227 | T/C |
| 3 | 43249022 | A/G |
| 3 | 43252564 | T/C |
| 3 | 43261296 | C/G |
| 3 | 43270277 | C/G |
| 3 | 43290571 | G/T |
| 3 | 43308554 | T/C |
| 3 | 43326468 | C/T |
| 3 | 43332286 | C/T |
| 3 | 43350409 | C/T |
| 3 | 43387151 | C/A |
| 3 | 43422959 | G/C |
| 3 | 43437024 | C/T |
| 3 | 43447532 | C/A |
| 3 | 43460135 | A/G |
| 3 | 43475615 | T/G |
| 3 | 43476850 | A/G |
| 3 | 43500364 | G/A |
| 3 | 43513108 | C/T |
| 3 | 43530479 | T/C |
| 3 | 43556935 | T/C |
| 3 | 43560235 | G/C |
| 3 | 43583332 | G/C |
| 3 | 43592832 | C/T |
| 3 | 43618010 | A/T |
| 3 | 43621118 | A/G |
| 3 | 43630972 | A/G |
| 3 | 43658340 | C/T |
| 3 | 43683646 | C/T |
| 3 | 43710843 | C/T |
| 3 | 43720585 | A/T |
| 3 | 43747746 | C/A |
| 3 | 43752870 | G/T |
| 3 | 43779310 | G/C |
| 3 | 43821712 | C/T |
| 3 | 43830681 | C/T |
| 3 | 43830682 | A/T |
| 3 | 43830683 | A/T |
| 3 | 43830684 | A/T |
| 3 | 43892311 | A/T |
| 3 | 43907711 | T/C |
| 3 | 43927718 | A/G |
| 3 | 43943442 | A/C |
| 3 | 43957314 | A/G |
| 3 | 43962852 | G/A |
| 3 | 43983782 | G/A |
| 3 | 44027660 | A/G |
| 3 | 44045007 | A/G |
| 3 | 44060242 | T/A |
| 3 | 44074980 | G/A |
| 3 | 44078168 | A/T |
| 3 | 44119710 | G/A |
| 3 | 44129451 | C/T |
| 3 | 44131860 | A/G |
| 3 | 44161108 | C/T |
| 3 | 44177569 | T/G |
| 3 | 44197750 | C/G |
| 3 | 44230337 | A/C |
| 3 | 44267148 | G/A |
| 3 | 44276579 | T/G |
| 3 | 44282279 | A/G |
| 3 | 44323870 | C/T |
| 3 | 44359124 | T/C |
| 3 | 44386521 | G/A |
| 3 | 44400682 | C/T |
| 3 | 44441881 | C/T |
| 3 | 44474519 | G/T |
| 3 | 44486213 | G/A |
| 3 | 44504012 | C/G |
| 3 | 44507954 | C/T |
| 3 | 44535592 | T/C |
| 3 | 44551709 | G/A |
| 3 | 44594684 | A/G |
| 3 | 44626946 | G/A |
| 3 | 44642150 | T/C |
| 3 | 44647408 | A/G |
| 3 | 44676381 | C/T |
| 3 | 44699722 | A/C |
| 3 | 44706449 | A/C |
| 3 | 44721146 | G/A |
| 3 | 44732261 | A/G |
| 3 | 44767500 | G/A |
| 3 | 44783979 | T/C |
| 3 | 44795377 | C/T |
| 3 | 44819910 | G/A |
| 3 | 44838665 | G/A |
| 3 | 44874484 | C/A |
| 3 | 44886673 | C/A |
| 3 | 44928912 | C/T |
| 3 | 44933439 | C/T |
| 3 | 44940476 | G/A |
| 3 | 44949919 | T/C |
| 3 | 44995602 | A/G |
| 3 | 44997791 | G/C |
| 3 | 45002324 | A/G |
| 3 | 45005561 | T/G |
| 3 | 45046367 | T/G |
| 3 | 45057942 | T/G |
| 3 | 45064863 | A/C |
| 3 | 45076289 | C/A |
| 3 | 45093264 | G/T |
| 3 | 45094738 | G/T |
| 3 | 45123611 | T/A |
| 3 | 45139490 | G/A |
| 3 | 45163035 | C/A |
| 3 | 45183596 | T/A |
| 3 | 45186238 | A/G |
| 3 | 45210508 | A/G |
| 3 | 45214005 | T/G |
| 3 | 45242769 | G/A |
| 3 | 45272987 | C/A |
| 3 | 45306461 | C/A |
| 3 | 45322498 | C/A |
| 3 | 45345434 | G/A |
| 3 | 45346852 | C/T |
| 3 | 45374347 | A/T |
| 3 | 45375390 | T/A |
| 3 | 45394738 | C/T |
| 3 | 45442550 | A/G |
| 3 | 45446115 | C/G |
| 3 | 45454343 | T/C |
| 3 | 45430116 | G/T |
| 3 | 45497042 | T/A |
| 3 | 45503343 | T/C |
| 3 | 45514776 | C/A |
| 3 | 45549503 | G/A |
| 3 | 45550871 | G/A |
| 3 | 45570990 | T/C |
| 3 | 45574398 | G/T |
| 3 | 45627027 | T/C |
| 3 | 45642077 | C/A |
| 3 | 45662179 | A/G |
| 3 | 45674472 | T/C |
| 3 | 45700678 | T/C |
| 3 | 45702922 | T/A |
| 3 | 45730767 | G/A |
| 3 | 45752598 | A/G |
| 3 | 45793757 | A/G |
| 3 | 45810086 | A/T |
| 3 | 45815705 | A/G |
| 3 | 45846774 | T/C |
| 3 | 45863771 | A/G |
| 3 | 45875165 | G/C |
| 3 | 45902568 | T/C |
| 3 | 45912663 | A/G |
| 3 | 45940359 | C/G |
| 3 | 45945137 | T/A |
| 3 | 45987915 | G/T |
| 3 | 45992057 | C/T |
| 3 | 46005989 | G/A |
| 3 | 46032243 | T/G |
| 3 | 46041363 | T/A |
| 3 | 46059831 | C/T |
| 3 | 46077729 | A/T |
| 3 | 46085938 | C/T |
| 3 | 46095365 | A/C |
| 3 | 46114163 | C/T |
| 3 | 46117679 | C/G |
| 3 | 46165820 | A/C |
| 3 | 46181080 | G/A |
| 3 | 46186512 | G/C |
| 3 | 46209447 | C/T |
| 3 | 46238742 | A/G |
| 3 | 46279829 | G/A |
| 3 | 46293242 | G/A |
| 3 | 46318239 | T/A |
| 3 | 46320190 | T/C |
| 3 | 46332675 | C/T |
| 3 | 46358197 | T/C |
| 3 | 46409325 | G/A |
| 3 | 46429239 | C/T |
| 3 | 46440779 | G/A |
| 3 | 46467641 | G/A |
| 3 | 46471215 | C/T |
| 3 | 46493840 | G/A |
| 3 | 46522469 | C/T |
| 3 | 46528926 | C/G |
| 3 | 46540217 | G/A |
| 3 | 46541497 | A/G |
| 3 | 46561528 | C/G |
| 3 | 46575008 | T/G |
| 3 | 46594048 | C/T |
| 3 | 46615311 | G/A |
| 3 | 46623043 | T/C |
| 3 | 46624405 | C/T |
| 3 | 46668801 | C/A |
| 3 | 46689218 | C/G |
| 3 | 46692371 | G/A |
| 3 | 46759490 | T/C |
| 3 | 46771165 | G/T |
| 3 | 46789716 | A/G |
| 3 | 46811512 | A/G |
| 3 | 46822757 | A/G |
| 3 | 46833677 | A/G |
| 3 | 46851383 | T/A |
| 3 | 46877616 | T/A |
| 3 | 46880731 | G/A |
| 3 | 46896586 | T/C |
| 3 | 46946863 | A/G |
| 3 | 46975026 | G/A |
| 3 | 46984660 | T/C |
| 3 | 47079650 | C/T |
| 3 | 47125767 | C/T |
| 3 | 47158782 | T/C |
| 3 | 47181057 | G/A |
| 3 | 47184874 | C/T |
| 3 | 47219941 | A/T |
| 3 | 47231555 | A/C |
| 3 | 47247124 | T/G |
| 3 | 47252834 | A/T |
| 3 | 47283466 | A/G |
| 3 | 47292254 | G/C |
| 3 | 47311272 | T/C |
| 3 | 47332804 | A/C |
| 3 | 47394841 | C/A |
| 3 | 47424317 | C/T |
| 3 | 47428140 | C/A |
| 3 | 47451332 | G/A |
| 3 | 47480535 | A/G |
| 3 | 47518938 | C/A |
| 3 | 47542670 | C/T |
| 3 | 47553074 | G/A |
| 3 | 47598225 | G/A |
| 3 | 47626719 | C/T |
| 3 | 47689223 | C/T |
| 3 | 47716044 | G/A |
| 3 | 47737106 | T/A |
| 3 | 47781881 | G/A |
| 3 | 47802132 | G/A |
| 3 | 47820764 | A/G |
| 3 | 47838203 | G/C |
| 3 | 47850475 | G/C |
| 3 | 47884547 | A/C |
| 3 | 47886517 | G/C |
| 3 | 47910140 | G/T |
| 3 | 47961688 | T/C |
| 3 | 47963087 | T/C |
| 3 | 47976383 | T/C |
| 3 | 48000134 | T/C |
| 3 | 48021355 | A/G |
| 3 | 48041374 | A/G |
| 3 | 48042492 | T/A |
| 3 | 48072064 | A/G |
| 3 | 48077122 | C/T |
| 3 | 48090058 | C/G |
| 3 | 48091783 | C/T |
| 3 | 48137787 | T/C |
| 3 | 48145935 | A/T |
| 3 | 48171778 | G/A |
| 3 | 48191924 | G/A |
| 3 | 48200345 | C/G |
| 3 | 48217180 | C/G |
| 3 | 48257268 | C/G |
| 3 | 48290480 | G/C |
| 3 | 48323308 | T/C |
| 4 | 28530 | C/T |
| 4 | 46090 | C/T |
| 4 | 47357 | A/T |
| 4 | 67554 | A/C |
| 4 | 86423 | C/A |
| 4 | 116587 | A/C |
| 4 | 118474 | T/C |
| 4 | 136623 | C/T |
| 4 | 140893 | C/T |
| 4 | 150347 | G/A |
| 4 | 157613 | T/C |
| 4 | 206801 | G/A |
| 4 | 224174 | T/C |
| 4 | 230236 | T/C |
| 4 | 248487 | A/G |
| 4 | 289823 | A/G |
| 4 | 351250 | C/T |
| 4 | 371549 | A/T |
| 4 | 372673 | C/T |
| 4 | 400977 | C/G |
| 4 | 430343 | C/G |
| 4 | 462550 | C/T |
| 4 | 485060 | T/C |
| 4 | 540124 | T/C |
| 4 | 547618 | T/C |
| 4 | 603874 | T/A |
| 4 | 635446 | T/C |
| 4 | 673687 | G/C |
| 4 | 744773 | C/A |
| 4 | 756364 | T/C |
| 4 | 780579 | G/T |
| 4 | 791341 | G/A |
| 4 | 818682 | G/A |
| 4 | 819997 | C/T |
| 4 | 864024 | G/C |
| 4 | 871610 | C/T |
| 4 | 1007352 | G/A |
| 4 | 1032045 | A/G |
| 4 | 1046659 | C/T |
| 4 | 1065627 | A/G |
| 4 | 1087636 | G/A |
| 4 | 1110677 | T/C |
| 4 | 1170687 | A/G |
| 4 | 1268861 | G/A |
| 4 | 1294223 | T/A |
| 4 | 1333198 | T/G |
| 4 | 1395406 | G/A |
| 4 | 1418377 | C/T |
| 4 | 1465854 | A/G |
| 4 | 1528717 | A/C |
| 4 | 1530226 | C/G |
| 4 | 1545185 | T/A |
| 4 | 1564242 | A/G |
| 4 | 1576075 | A/G |
| 4 | 1607799 | T/C |
| 4 | 1633503 | C/A |
| 4 | 1660769 | T/C |
| 4 | 1665305 | G/T |
| 4 | 1703979 | G/A |
| 4 | 1741218 | A/G |
| 4 | 1798504 | G/A |
| 4 | 1827112 | C/A |
| 4 | 1840909 | T/G |
| 4 | 1855560 | T/C |
| 4 | 1855561 | A/C |
| 4 | 1855562 | A/C |
| 4 | 1855563 | A/C |
| 4 | 1913297 | T/G |
| 4 | 1935555 | G/A |
| 4 | 1987226 | A/G |
| 4 | 2023429 | T/C |
| 4 | 2036587 | T/C |
| 4 | 2082546 | C/T |
| 4 | 2091578 | A/G |
| 4 | 2126559 | T/C |
| 4 | 2143916 | G/A |
| 4 | 2192541 | T/C |
| 4 | 2200228 | T/C |
| 4 | 2229249 | G/A |
| 4 | 2275029 | T/C |
| 4 | 2292720 | G/T |
| 4 | 2309953 | G/C |
| 4 | 2321804 | G/T |
| 4 | 2333073 | G/A |
| 4 | 2357452 | C/T |
| 4 | 2390708 | G/A |
| 4 | 2414362 | T/G |
| 4 | 2430376 | G/A |
| 4 | 2558559 | C/A |
| 4 | 2605960 | C/T |
| 4 | 2643622 | C/T |
| 4 | 2680266 | C/T |
| 4 | 2689065 | T/C |
| 4 | 2704683 | A/G |
| 4 | 2775823 | T/C |
| 4 | 2813792 | G/C |
| 4 | 2815888 | G/A |
| 4 | 2900671 | C/A |
| 4 | 2914848 | C/T |
| 4 | 3020162 | A/G |
| 4 | 3074006 | C/A |
| 4 | 3079828 | C/T |
| 4 | 3130983 | C/G |
| 4 | 3136540 | T/C |
| 4 | 3185361 | C/G |
| 4 | 3207810 | G/C |
| 4 | 3248569 | T/C |
| 4 | 3286641 | C/T |
| 4 | 3371569 | A/G |
| 4 | 3476296 | T/G |
| 4 | 3521996 | G/A |
| 4 | 3532815 | G/A |
| 4 | 3563040 | G/T |
| 4 | 3595816 | A/G |
| 4 | 3686248 | C/T |
| 4 | 3719937 | T/C |
| 4 | 3802732 | A/G |
| 4 | 3861930 | C/T |
| 4 | 3912583 | A/G |
| 4 | 3955285 | A/G |
| 4 | 3984155 | G/A |
| 4 | 3997002 | G/A |
| 4 | 4003748 | C/T |
| 4 | 4028083 | C/T |
| 4 | 4057188 | C/T |
| 4 | 4134563 | G/A |
| 4 | 4136857 | T/C |
| 4 | 4155771 | T/A |
| 4 | 4175585 | G/A |
| 4 | 4205287 | G/T |
| 4 | 4237694 | G/T |
| 4 | 4274997 | T/G |
| 4 | 4304276 | G/A |
| 4 | 4324614 | G/A |
| 4 | 4364839 | T/C |
| 4 | 4374439 | C/T |
| 4 | 4407834 | A/G |
| 4 | 4465352 | G/T |
| 4 | 4468631 | C/T |
| 4 | 4494783 | A/T |
| 4 | 4496557 | G/C |
| 4 | 4528274 | G/A |
| 4 | 4530611 | A/G |
| 4 | 4533508 | C/G |
| 4 | 4565274 | C/T |
| 4 | 4594644 | T/C |
| 4 | 4611692 | C/T |
| 4 | 4632390 | G/A |
| 4 | 4676078 | A/G |
| 4 | 4685426 | C/T |
| 4 | 4713810 | G/A |
| 4 | 4746398 | G/C |
| 4 | 4761269 | T/C |
| 4 | 4777084 | A/G |
| 4 | 4802427 | G/C |
| 4 | 4809919 | T/C |
| 4 | 4835901 | A/T |
| 4 | 4840394 | A/C |
| 4 | 4871673 | C/T |
| 4 | 4905679 | A/T |
| 4 | 4949800 | T/C |
| 4 | 4976824 | G/A |
| 4 | 4985028 | G/A |
| 4 | 5037225 | A/G |
| 4 | 5094322 | A/G |
| 4 | 5117451 | T/A |
| 4 | 5124044 | A/T |
| 4 | 5149867 | A/C |
| 4 | 5151623 | C/T |
| 4 | 5165005 | A/G |
| 4 | 5184210 | T/C |
| 4 | 5194095 | T/C |
| 4 | 5201944 | G/A |
| 4 | 5226063 | T/A |
| 4 | 5262624 | G/A |
| 4 | 5330887 | A/C |
| 4 | 5334383 | C/T |
| 4 | 5364578 | T/G |
| 4 | 5388991 | G/A |
| 4 | 5416629 | C/T |
| 4 | 5443240 | A/C |
| 4 | 5477404 | C/T |
| 4 | 5515872 | C/A |
| 4 | 5517352 | T/C |
| 4 | 5521787 | T/A |
| 4 | 5544988 | T/G |
| 4 | 5551808 | T/C |
| 4 | 5602575 | G/A |
| 4 | 5604147 | C/T |
| 4 | 5622487 | C/T |
| 4 | 5667944 | T/C |
| 4 | 5695697 | T/C |
| 4 | 5697211 | C/T |
| 4 | 5713421 | T/C |
| 4 | 5736480 | G/A |
| 4 | 5748660 | C/T |
| 4 | 5766208 | T/G |
| 4 | 5809544 | A/G |
| 4 | 5810801 | C/G |
| 4 | 5821247 | G/C |
| 4 | 5866986 | C/T |
| 4 | 5884007 | T/A |
| 4 | 5897744 | A/T |
| 4 | 5911034 | T/G |
| 4 | 5929474 | T/G |
| 4 | 5948555 | G/A |
| 4 | 5953715 | C/T |
| 4 | 5979500 | G/A |
| 4 | 5984604 | T/G |
| 4 | 6026879 | C/T |
| 4 | 6052247 | G/A |
| 4 | 6080117 | C/T |
| 4 | 6081777 | A/G |
| 4 | 6132137 | T/C |
| 4 | 6138560 | G/A |
| 4 | 6159658 | T/G |
| 4 | 6183683 | T/G |
| 4 | 6209101 | T/G |
| 4 | 6210385 | G/C |
| 4 | 6261906 | T/G |
| 4 | 6265383 | A/G |
| 4 | 6340342 | A/C |
| 4 | 3630427 | A/G |
| 4 | 6401079 | A/G |
| 4 | 6426226 | C/T |
| 4 | 6428423 | C/T |
| 4 | 6450283 | T/C |
| 4 | 6457325 | G/A |
| 4 | 6498809 | A/G |
| 4 | 6510058 | G/T |
| 4 | 6515273 | G/A |
| 4 | 6545810 | A/G |
| 4 | 6580827 | G/C |
| 4 | 6604151 | G/A |
| 4 | 6619792 | T/A |
| 4 | 6644310 | T/A |
| 4 | 6718327 | A/T |
| 4 | 6810141 | T/C |
| 4 | 6829991 | A/C |
| 4 | 6868222 | C/T |
| 4 | 6875230 | C/T |
| 4 | 6892949 | C/T |
| 4 | 6902534 | T/C |
| 4 | 6910966 | G/T |
| 4 | 6933885 | C/T |
| 4 | 6955719 | G/A |
| 4 | 6973674 | C/A |
| 4 | 6990428 | A/G |
| 4 | 6996087 | G/T |
| 4 | 7027502 | A/G |
| 4 | 7029415 | C/T |
| 4 | 7070145 | G/A |
| 4 | 7109283 | G/C |
| 4 | 7119500 | T/C |
| 4 | 7128759 | T/C |
| 4 | 7141049 | C/T |
| 4 | 7162744 | C/T |
| 4 | 7195386 | T/A |
| 4 | 7201575 | T/C |
| 4 | 7254586 | A/G |
| 4 | 7261119 | C/T |
| 4 | 7307974 | G/A |
| 4 | 7324699 | T/C |
| 4 | 7353268 | T/C |
| 4 | 7357631 | A/G |
| 4 | 7396640 | G/A |
| 4 | 7409463 | G/A |
| 4 | 7438813 | G/A |
| 4 | 7440913 | A/T |
| 4 | 7455944 | C/G |
| 4 | 7473827 | G/T |
| 4 | 7474923 | T/C |
| 4 | 7523523 | G/C |
| 4 | 7541988 | A/G |
| 4 | 7551673 | A/C |
| 4 | 7574723 | A/G |
| 4 | 7595468 | G/A |
| 4 | 7607221 | G/A |
| 4 | 7637980 | C/A |
| 4 | 7644372 | T/C |
| 4 | 7666666 | A/T |
| 4 | 7677188 | T/C |
| 4 | 7694472 | T/C |
| 4 | 7715526 | C/T |
| 4 | 7725164 | G/A |
| 4 | 7781532 | G/A |
| 4 | 7801493 | G/A |
| 4 | 7816827 | A/G |
| 4 | 7839672 | T/G |
| 4 | 7840764 | G/A |
| 4 | 7874527 | A/G |
| 4 | 7879790 | A/T |
| 4 | 7890323 | T/G |
| 4 | 7930769 | G/A |
| 4 | 7940706 | C/T |
| 4 | 7950030 | C/A |
| 4 | 7957725 | T/C |
| 4 | 7989505 | G/A |
| 4 | 8007112 | C/A |
| 4 | 8035596 | C/G |
| 4 | 8052676 | G/T |
| 4 | 8060705 | A/T |
| 4 | 8077249 | G/C |
| 4 | 8087618 | G/T |
| 4 | 8103302 | G/A |
| 4 | 8139636 | A/G |
| 4 | 8146765 | C/T |
| 4 | 8166062 | T/C |
| 4 | 8172230 | A/C |
| 4 | 8191531 | A/G |
| 4 | 8228362 | T/A |
| 4 | 8268080 | G/A |
| 4 | 8279545 | G/A |
| 4 | 8293795 | T/C |
| 4 | 8308584 | C/A |
| 4 | 8319302 | C/G |
| 4 | 8356741 | C/T |
| 4 | 8364975 | C/T |
| 4 | 8370231 | A/G |
| 4 | 8389701 | C/T |
| 4 | 8427183 | C/T |
| 4 | 8428502 | T/C |
| 4 | 8430802 | T/G |
| 4 | 8440507 | C/G |
| 4 | 8463665 | G/C |
| 4 | 8496565 | C/G |
| 4 | 8502867 | G/A |
| 4 | 8526152 | A/T |
| 4 | 5835773 | T/C |
| 4 | 8550370 | A/G |
| 4 | 8562300 | A/T |
| 4 | 8581041 | A/T |
| 4 | 8611576 | A/T |
| 4 | 8627338 | G/C |
| 4 | 8667411 | T/C |
| 4 | 8673395 | G/A |
| 4 | 8689094 | G/T |
| 4 | 8708969 | T/C |
| 4 | 8724509 | A/C |
| 4 | 8743771 | T/A |
| 4 | 8769439 | C/G |
| 4 | 8784729 | C/A |
| 4 | 8797255 | C/T |
| 4 | 8810990 | A/T |
| 4 | 8823400 | G/C |
| 4 | 8845528 | C/T |
| 4 | 8852153 | G/T |
| 4 | 8863431 | T/C |
| 4 | 8884648 | T/C |
| 4 | 8890411 | C/T |
| 4 | 8910985 | G/A |
| 4 | 8938155 | C/T |
| 4 | 8940455 | G/A |
| 4 | 8951786 | T/G |
| 4 | 8970213 | G/C |
| 4 | 8989179 | C/A |
| 4 | 9009249 | G/A |
| 4 | 9038071 | G/A |
| 4 | 9042465 | A/G |
| 4 | 9080261 | G/A |
| 4 | 9085010 | C/T |
| 4 | 9092118 | T/A |
| 4 | 9118825 | A/T |
| 4 | 9120046 | A/G |
| 4 | 9160373 | A/C |
| 4 | 9170521 | G/A |
| 4 | 9182878 | C/T |
| 4 | 9197314 | C/T |
| 4 | 9220310 | A/G |
| 4 | 9222527 | T/G |
| 4 | 9244007 | C/T |
| 4 | 9246103 | A/G |
| 4 | 9270872 | A/G |
| 4 | 9290444 | T/C |
| 4 | 9306112 | C/G |
| 4 | 9346821 | A/T |
| 4 | 9350022 | A/T |
| 4 | 9387231 | T/G |
| 4 | 9416555 | C/G |
| 4 | 9437855 | T/G |
| 4 | 9466615 | A/G |
| 4 | 9475367 | G/A |
| 4 | 9485670 | C/T |
| 4 | 9496196 | C/T |
| 4 | 9520528 | A/C |
| 4 | 9552806 | G/T |
| 4 | 9554533 | A/G |
| 4 | 9580381 | G/A |
| 4 | 9591525 | A/T |
| 4 | 9609758 | C/T |
| 4 | 9644301 | G/C |
| 4 | 9646671 | A/G |
| 4 | 9664936 | G/A |
| 4 | 9677516 | C/G |
| 4 | 9693524 | T/G |
| 4 | 9699108 | C/T |
| 4 | 9735579 | C/T |
| 4 | 9762869 | G/A |
| 4 | 9765175 | G/A |
| 4 | 9803621 | G/A |
| 4 | 9835253 | G/A |
| 4 | 9840615 | C/T |
| 4 | 9891158 | G/T |
| 4 | 9914874 | G/A |
| 4 | 9918426 | A/G |
| 4 | 9951511 | T/C |
| 4 | 10002523 | G/A |
| 4 | 10024230 | T/C |
| 4 | 10045586 | C/T |
| 4 | 10054963 | T/C |
| 4 | 10078278 | G/C |
| 4 | 10088112 | T/C |
| 4 | 10093160 | T/C |
| 4 | 10110967 | G/A |
| 4 | 10193336 | G/T |
| 4 | 10194738 | T/C |
| 4 | 10222154 | T/C |
| 4 | 10302141 | A/G |
| 4 | 10316401 | G/A |
| 4 | 10327480 | G/A |
| 4 | 10343326 | G/A |
| 4 | 10370186 | C/A |
| 4 | 10371892 | G/A |
| 4 | 10399688 | G/A |
| 4 | 10430756 | C/T |
| 4 | 10444280 | C/G |
| 4 | 10495142 | G/A |
| 4 | 10497485 | C/T |
| 4 | 10506498 | C/T |
| 4 | 10506499 | A/G |
| 4 | 10506500 | A/G |
| 4 | 10506501 | A/G |
| 4 | 10618383 | G/A |
| 4 | 10629790 | C/T |
| 4 | 10640202 | T/A |
| 4 | 10654184 | T/C |
| 4 | 10656789 | C/A |
| 4 | 10681697 | G/A |
| 4 | 10685457 | C/T |
| 4 | 10711998 | G/A |
| 4 | 10723247 | T/A |
| 4 | 10745464 | G/C |
| 4 | 10764279 | G/A |
| 4 | 10770562 | C/T |
| 4 | 10810125 | C/A |
| 4 | 10831010 | C/G |
| 4 | 10839154 | C/T |
| 4 | 10866137 | G/A |
| 4 | 10871861 | G/A |
| 4 | 10906025 | A/G |
| 4 | 10943129 | C/T |
| 4 | 10962043 | A/C |
| 4 | 10992558 | C/G |
| 4 | 11048904 | C/T |
| 4 | 11066036 | T/C |
| 4 | 11070991 | G/A |
| 4 | 11114310 | G/A |
| 4 | 11209814 | A/G |
| 4 | 11241212 | A/G |
| 4 | 11254124 | A/G |
| 4 | 11290268 | T/C |
| 4 | 11295979 | T/C |
| 4 | 11315018 | T/C |
| 4 | 11319926 | C/G |
| 4 | 11401612 | C/T |
| 4 | 11474784 | G/A |
| 4 | 11505835 | G/A |
| 4 | 11537325 | G/T |
| 4 | 11561640 | A/T |
| 4 | 11602141 | C/T |
| 4 | 11610442 | G/A |
| 4 | 11611556 | T/C |
| 4 | 11641754 | A/G |
| 4 | 11656811 | C/G |
| 4 | 11720347 | C/T |
| 4 | 11731424 | G/A |
| 4 | 11765211 | C/T |
| 4 | 11787649 | C/T |
| 4 | 11812444 | C/T |
| 4 | 11818407 | G/A |
| 4 | 11828223 | G/A |
| 4 | 11879217 | A/G |
| 4 | 11897542 | G/A |
| 4 | 11913405 | T/C |
| 4 | 11961225 | C/G |
| 4 | 11996060 | G/A |
| 4 | 12000844 | C/T |
| 4 | 12033367 | T/A |
| 4 | 12033368 | T/A |
| 4 | 12033369 | T/A |
| 4 | 12091612 | T/C |
| 4 | 12120176 | T/C |
| 4 | 12153264 | C/T |
| 4 | 12165299 | A/G |
| 4 | 12181633 | A/T |
| 4 | 12196295 | A/C |
| 4 | 12210531 | A/C |
| 4 | 12254387 | G/A |
| 4 | 12264813 | G/A |
| 4 | 12276148 | T/C |
| 4 | 12324092 | C/T |
| 4 | 12327889 | A/C |
| 4 | 12332984 | T/A |
| 4 | 12387127 | T/C |
| 4 | 12389189 | C/A |
| 4 | 12403666 | A/G |
| 4 | 12437100 | G/A |
| 4 | 12466114 | A/G |
| 4 | 12491112 | A/G |
| 4 | 12535088 | G/A |
| 4 | 12550237 | G/T |
| 4 | 12576483 | T/A |
| 4 | 12588567 | C/T |
| 4 | 12616262 | G/A |
| 4 | 12626789 | C/G |
| 4 | 12630684 | T/C |
| 4 | 12664430 | A/G |
| 4 | 12692264 | A/G |
| 4 | 12709544 | T/C |
| 4 | 12722937 | A/T |
| 4 | 12724120 | T/C |
| 4 | 12753847 | C/T |
| 4 | 12756442 | G/A |
| 4 | 12781301 | A/T |
| 4 | 12794723 | C/A |
| 4 | 12851831 | G/A |
| 4 | 12870480 | G/A |
| 4 | 12883394 | G/A |
| 4 | 12900956 | G/T |
| 4 | 12938694 | G/A |
| 4 | 12947371 | G/A |
| 4 | 12968221 | T/A |
| 4 | 12976555 | G/A |
| 4 | 13002331 | T/C |
| 4 | 13020223 | G/T |
| 4 | 13068607 | T/C |
| 4 | 13110585 | A/C |
| 4 | 13127893 | A/G |
| 4 | 13145951 | A/G |
| 4 | 13157485 | A/G |
| 4 | 13198261 | C/T |
| 4 | 13206121 | G/A |
| 4 | 13219434 | G/A |
| 4 | 13235148 | G/A |
| 4 | 13310197 | A/T |
| 4 | 13350699 | T/G |
| 4 | 13395440 | C/T |
| 4 | 13422592 | C/T |
| 4 | 13457025 | C/T |
| 4 | 13516544 | C/T |
| 4 | 13534760 | C/T |
| 4 | 13544582 | G/A |
| 4 | 13575514 | T/C |
| 4 | 13591714 | G/A |
| 4 | 13622689 | C/T |
| 4 | 13626770 | G/A |
| 4 | 13674639 | C/T |
| 4 | 13732538 | T/G |
| 4 | 13896317 | G/A |
| 4 | 13901370 | A/G |
| 4 | 13922483 | G/A |
| 4 | 13930965 | C/T |
| 4 | 13959968 | G/A |
| 4 | 13992075 | C/G |
| 4 | 14070958 | G/A |
| 4 | 14136375 | T/C |
| 4 | 14192520 | T/C |
| 4 | 14242471 | C/G |
| 4 | 14269006 | C/T |
| 4 | 14275491 | T/C |
| 4 | 14288432 | G/A |
| 4 | 14339017 | G/A |
| 4 | 14370698 | G/A |
| 4 | 14402138 | G/A |
| 4 | 14436193 | A/G |
| 4 | 14455093 | G/A |
| 4 | 14466997 | C/T |
| 4 | 14490751 | T/C |
| 4 | 14497957 | C/A |
| 4 | 14567343 | A/G |
| 4 | 14606076 | G/T |
| 4 | 14618918 | A/C |
| 4 | 14656107 | G/A |
| 4 | 14683786 | C/A |
| 4 | 14714114 | C/T |
| 4 | 14779893 | G/A |
| 4 | 14782367 | C/T |
| 4 | 14838412 | T/C |
| 4 | 14844457 | G/A |
| 4 | 14898368 | C/G |
| 4 | 14926077 | G/T |
| 4 | 14971285 | C/T |
| 4 | 14987701 | A/G |
| 4 | 15018199 | T/G |
| 4 | 15026654 | T/A |
| 4 | 15053988 | T/G |
| 4 | 15084564 | G/A |
| 4 | 15107305 | C/T |
| 4 | 15118322 | G/A |
| 4 | 15132270 | C/T |
| 4 | 15137653 | C/T |
| 4 | 15165224 | C/T |
| 4 | 15209134 | A/T |
| 4 | 15210489 | T/A |
| 4 | 15230150 | C/G |
| 4 | 15260139 | G/T |
| 4 | 15262052 | C/G |
| 4 | 15270671 | A/G |
| 4 | 15283998 | C/T |
| 4 | 15353126 | G/A |
| 4 | 15356355 | G/A |
| 4 | 15386565 | G/A |
| 4 | 15400163 | C/T |
| 4 | 15401638 | G/A |
| 4 | 15439222 | C/T |
| 4 | 15452641 | C/T |
| 4 | 15459273 | T/C |
| 4 | 15480586 | G/A |
| 4 | 15510617 | G/T |
| 4 | 15519339 | C/T |
| 4 | 15547933 | T/C |
| 4 | 15553949 | A/G |
| 4 | 15588036 | C/A |
| 4 | 15621768 | T/C |
| 4 | 15631134 | T/C |
| 4 | 15675191 | G/A |
| 4 | 15689402 | G/A |
| 4 | 15711224 | G/A |
| 4 | 15712549 | G/T |
| 4 | 15720561 | A/C |
| 4 | 15732093 | T/C |
| 4 | 15756555 | A/T |
| 4 | 15766904 | C/T |
| 4 | 15781517 | A/T |
| 4 | 15788716 | C/A |
| 4 | 15812185 | G/A |
| 4 | 15817719 | C/T |
| 4 | 15862235 | A/G |
| 4 | 15865625 | C/T |
| 4 | 15886902 | C/T |
| 4 | 15932149 | C/T |
| 4 | 15938203 | T/A |
| 4 | 15964984 | C/T |
| 4 | 15981488 | A/G |
| 4 | 16002720 | G/A |
| 4 | 16036214 | T/C |
| 4 | 16049216 | G/A |
| 4 | 16053052 | T/A |
| 4 | 16065765 | G/A |
| 4 | 16097313 | C/T |
| 4 | 16116998 | G/A |
| 4 | 16137385 | C/T |
| 4 | 16150771 | G/A |
| 4 | 16171098 | G/A |
| 4 | 16201474 | G/T |
| 4 | 16215916 | G/A |
| 4 | 16230723 | C/T |
| 4 | 16284131 | A/T |
| 4 | 16294165 | A/C |
| 4 | 16345350 | G/A |
| 4 | 16361049 | C/T |
| 4 | 16365746 | G/A |
| 4 | 16365747 | T/G |
| 4 | 16365748 | T/G |
| 4 | 16365749 | T/G |
| 4 | 16425758 | G/A |
| 4 | 16446810 | T/C |
| 4 | 16454859 | G/A |
| 4 | 16497953 | G/T |
| 4 | 16512902 | G/A |
| 4 | 16524884 | G/T |
| 4 | 16558160 | C/A |
| 4 | 16566174 | A/G |
| 4 | 16573850 | A/G |
| 4 | 16007186 | A/G |
| 4 | 16617897 | A/T |
| 4 | 16643007 | C/T |
| 4 | 16647902 | C/A |
| 4 | 16655650 | G/A |
| 4 | 16677251 | C/G |
| 4 | 16701357 | C/T |
| 4 | 16707374 | C/G |
| 4 | 16723027 | A/G |
| 4 | 16746249 | T/C |
| 4 | 16756945 | G/A |
| 4 | 16789533 | T/A |
| 4 | 16804264 | G/A |
| 4 | 16827625 | G/A |
| 4 | 16849486 | A/T |
| 4 | 16852800 | G/A |
| 4 | 16861369 | A/G |
| 4 | 16873203 | G/A |
| 4 | 16897241 | G/A |
| 4 | 16920950 | C/A |
| 4 | 16938150 | T/C |
| 4 | 16953927 | G/A |
| 4 | 16985684 | G/A |
| 4 | 17003925 | C/T |
| 4 | 17038421 | C/T |
| 4 | 17098989 | C/T |
| 4 | 17105582 | G/A |
| 4 | 17124356 | C/T |
| 4 | 17167187 | C/T |
| 4 | 17179860 | G/T |
| 4 | 17205281 | C/A |
| 4 | 17212184 | G/C |
| 4 | 17226226 | G/A |
| 4 | 17244161 | G/T |
| 4 | 17262355 | C/A |
| 4 | 17266773 | A/G |
| 4 | 17291315 | A/G |
| 4 | 17292859 | G/A |
| 4 | 17327012 | A/G |
| 4 | 17333241 | G/A |
| 4 | 17358383 | C/A |
| 4 | 17370666 | C/G |
| 4 | 17421319 | T/C |
| 4 | 17422455 | T/C |
| 4 | 17431923 | T/G |
| 4 | 17445045 | G/A |
| 4 | 17473319 | G/A |
| 4 | 17474409 | G/A |
| 4 | 17494106 | T/C |
| 4 | 17518606 | T/G |
| 4 | 17523936 | C/T |
| 4 | 17564560 | A/G |
| 4 | 17570866 | T/C |
| 4 | 17592110 | T/C |
| 4 | 17595319 | T/C |
| 4 | 17627994 | G/A |
| 4 | 17636883 | C/T |
| 4 | 17644482 | C/T |
| 4 | 17653792 | G/T |
| 4 | 17685448 | A/T |
| 4 | 17696233 | A/G |
| 4 | 17743452 | T/C |
| 4 | 17445001 | C/T |
| 4 | 17774452 | C/T |
| 4 | 17817537 | A/G |
| 4 | 17834950 | C/T |
| 4 | 17840132 | C/A |
| 4 | 17853678 | G/C |
| 4 | 17868802 | G/A |
| 4 | 17896425 | T/G |
| 4 | 17902020 | C/T |
| 4 | 17913604 | G/A |
| 4 | 17953991 | C/T |
| 4 | 17962045 | G/A |
| 4 | 17980645 | T/C |
| 4 | 18009091 | G/C |
| 4 | 18051807 | C/T |
| 4 | 18067620 | C/T |
| 4 | 18085264 | G/T |
| 4 | 18090922 | G/A |
| 4 | 18145845 | A/G |
| 4 | 18152618 | G/A |
| 4 | 18155970 | A/G |
| 4 | 18208209 | T/C |
| 4 | 18209706 | A/T |
| 4 | 18224856 | G/T |
| 4 | 18236562 | C/T |
| 4 | 18256997 | G/A |
| 4 | 18261192 | A/G |
| 4 | 18275129 | T/C |
| 4 | 18282007 | A/G |
| 4 | 18300355 | C/T |
| 4 | 18323711 | G/A |
| 4 | 18330776 | C/A |
| 4 | 18341524 | G/A |
| 4 | 18360451 | A/G |
| 4 | 18362359 | C/T |
| 4 | 18391924 | G/T |
| 4 | 18410921 | G/A |
| 4 | 18422650 | A/C |
| 4 | 18468148 | C/T |
| 4 | 18475415 | G/A |
| 4 | 18482247 | G/A |
| 4 | 18522508 | A/G |
| 4 | 18568389 | T/C |
| 4 | 18573883 | A/G |
| 4 | 18603748 | A/T |
| 4 | 18617931 | A/C |
| 4 | 18631769 | A/G |
| 4 | 18659196 | T/C |
| 4 | 18663654 | T/C |
| 4 | 18669955 | A/C |
| 4 | 18713469 | G/A |
| 4 | 18720847 | G/A |
| 4 | 18723812 | C/T |
| 4 | 18770027 | G/A |
| 4 | 18807478 | A/G |
| 4 | 18818232 | G/C |
| 4 | 18869994 | A/G |
| 4 | 18885544 | A/C |
| 4 | 18887359 | A/T |
| 4 | 18903498 | G/A |
| 4 | 18944457 | G/A |
| 4 | 18954121 | A/G |
| 4 | 18960553 | G/C |
| 4 | 18966189 | C/T |
| 4 | 18992842 | A/G |
| 4 | 18999895 | A/G |
| 4 | 19024084 | T/G |
| 4 | 19040971 | G/A |
| 4 | 19070872 | C/T |
| 4 | 19074769 | C/T |
| 4 | 19095814 | G/A |
| 4 | 19105832 | A/C |
| 4 | 19122895 | G/T |
| 4 | 19154178 | T/C |
| 4 | 19164106 | C/G |
| 4 | 19171477 | T/C |
| 4 | 19215408 | A/C |
| 4 | 19219561 | T/C |
| 4 | 19243859 | A/C |
| 4 | 19260888 | T/C |
| 4 | 19263866 | C/T |
| 4 | 19290013 | A/C |
| 4 | 19294783 | G/A |
| 4 | 19320147 | G/T |
| 4 | 19351358 | C/A |
| 4 | 19363413 | C/A |
| 4 | 19400637 | G/A |
| 4 | 19424211 | C/T |
| 4 | 19467772 | C/T |
| 4 | 19471509 | C/G |
| 4 | 19489731 | C/A |
| 4 | 19501413 | C/T |
| 4 | 19506639 | G/A |
| 4 | 19556165 | C/A |
| 4 | 19650278 | C/T |
| 4 | 19704979 | T/C |
| 4 | 19710042 | A/C |
| 4 | 19715976 | T/C |
| 4 | 19755736 | G/A |
| 4 | 19764395 | G/A |
| 4 | 19787251 | G/A |
| 4 | 19816188 | T/C |
| 4 | 19827119 | T/A |
| 4 | 19832344 | T/G |
| 4 | 19843193 | A/T |
| 4 | 19906050 | C/T |
| 4 | 19943366 | A/G |
| 4 | 19971211 | G/A |
| 4 | 19977284 | T/C |
| 4 | 19993679 | G/A |
| 4 | 20010045 | T/C |
| 4 | 20032285 | G/A |
| 4 | 20051408 | A/T |
| 4 | 20075441 | G/A |
| 4 | 20111238 | C/T |
| 4 | 20122785 | C/T |
| 4 | 20147307 | A/G |
| 4 | 20152696 | A/G |
| 4 | 20161593 | A/T |
| 4 | 20173393 | G/A |
| 4 | 20211428 | C/T |
| 4 | 20220263 | G/A |
| 4 | 20226826 | G/A |
| 4 | 20260850 | C/T |
| 4 | 20265669 | T/C |
| 4 | 20304254 | C/T |
| 4 | 20310001 | C/T |
| 4 | 20343421 | T/C |
| 4 | 20365720 | T/C |
| 4 | 20383429 | A/G |
| 4 | 20396054 | C/T |
| 4 | 20418903 | G/C |
| 4 | 20442575 | G/A |
| 4 | 20468729 | A/G |
| 4 | 20473855 | A/G |
| 4 | 20518134 | A/G |
| 4 | 20520525 | G/A |
| 4 | 20531204 | G/A |
| 4 | 20572479 | G/A |
| 4 | 20576131 | C/T |
| 4 | 20580103 | G/T |
| 4 | 20595745 | G/A |
| 4 | 20610208 | G/A |
| 4 | 20610209 | A/G |
| 4 | 20610210 | A/G |
| 4 | 20670766 | A/G |
| 4 | 20688913 | C/T |
| 4 | 20711048 | T/C |
| 4 | 20725674 | C/T |
| 4 | 20865856 | A/G |
| 4 | 20885175 | T/C |
| 4 | 20917749 | G/A |
| 4 | 20923174 | C/G |
| 4 | 20943596 | T/C |
| 4 | 20974634 | A/G |
| 4 | 20989739 | T/C |
| 4 | 21000739 | C/T |
| 4 | 21007389 | A/C |
| 4 | 21055073 | A/C |
| 4 | 21060386 | C/T |
| 4 | 21062401 | T/A |
| 4 | 21097304 | G/A |
| 4 | 21114659 | T/A |
| 4 | 21136109 | C/A |
| 4 | 21142505 | T/C |
| 4 | 21163141 | T/C |
| 4 | 21165276 | A/G |
| 4 | 21189826 | T/C |
| 4 | 21191800 | C/A |
| 4 | 21230540 | T/C |
| 4 | 21261597 | C/T |
| 4 | 21276794 | G/C |
| 4 | 21299057 | G/A |
| 4 | 21309290 | A/G |
| 4 | 21332314 | C/T |
| 4 | 21333976 | C/T |
| 4 | 21364043 | C/A |
| 4 | 21369464 | T/C |
| 4 | 21402926 | T/C |
| 4 | 21418884 | T/C |
| 4 | 21421855 | C/T |
| 4 | 21423458 | T/G |
| 4 | 21474876 | C/T |
| 4 | 21501374 | C/T |
| 4 | 21513943 | G/C |
| 4 | 21517435 | A/G |
| 4 | 21543715 | A/G |
| 4 | 21548814 | A/G |
| 4 | 21596568 | C/T |
| 4 | 21658199 | C/G |
| 4 | 21664063 | C/A |
| 4 | 21685356 | A/T |
| 4 | 21795691 | A/G |
| 4 | 21802711 | C/T |
| 4 | 21818486 | G/A |
| 4 | 21826255 | A/T |
| 4 | 21841056 | G/A |
| 4 | 21847145 | A/G |
| 4 | 21870055 | T/C |
| 4 | 21878941 | A/G |
| 4 | 21902291 | C/G |
| 4 | 21931671 | C/G |
| 4 | 21960499 | C/A |
| 4 | 21964475 | C/G |
| 4 | 22000137 | A/G |
| 4 | 22011125 | A/G |
| 4 | 22041404 | G/A |
| 4 | 22063915 | G/A |
| 4 | 22072091 | T/C |
| 4 | 22090322 | T/C |
| 4 | 22100303 | C/T |
| 4 | 22117882 | G/A |
| 4 | 22138518 | T/C |
| 4 | 22146036 | C/T |
| 4 | 22164598 | A/G |
| 4 | 22176227 | A/G |
| 4 | 22186730 | C/T |
| 4 | 22211270 | G/A |
| 4 | 22213214 | C/T |
| 4 | 22233563 | A/G |
| 4 | 22262364 | T/C |
| 4 | 22288689 | C/A |
| 4 | 22304078 | T/A |
| 4 | 22316453 | A/G |
| 4 | 22320094 | G/A |
| 4 | 22331865 | T/G |
| 4 | 22367083 | G/A |
| 4 | 22377208 | T/C |
| 4 | 22392353 | T/C |
| 4 | 22430031 | T/C |
| 4 | 22440153 | T/G |
| 4 | 22454383 | A/G |
| 4 | 22487183 | T/A |
| 4 | 22488280 | C/A |
| 4 | 22500544 | A/G |
| 4 | 22548231 | G/A |
| 4 | 22556716 | A/G |
| 4 | 22588673 | T/C |
| 4 | 22607607 | C/G |
| 4 | 22631803 | G/A |
| 4 | 22648563 | C/T |
| 4 | 22651288 | T/C |
| 4 | 22684597 | G/A |
| 4 | 22721026 | A/G |
| 4 | 22742387 | G/A |
| 4 | 22781933 | G/A |
| 4 | 22783223 | C/A |
| 4 | 22815430 | A/T |
| 4 | 22827698 | T/C |
| 4 | 22849509 | A/G |
| 4 | 22866901 | T/G |
| 4 | 22880616 | C/A |
| 4 | 22897859 | T/C |
| 4 | 22910609 | T/C |
| 4 | 22932450 | A/G |
| 4 | 22954579 | C/T |
| 4 | 22955664 | A/G |
| 4 | 23008348 | G/A |
| 4 | 23020676 | A/C |
| 4 | 23036204 | A/G |
| 4 | 23062443 | T/G |
| 4 | 23074202 | G/T |
| 4 | 23105855 | T/C |
| 4 | 23139224 | T/G |
| 4 | 23157692 | G/C |
| 4 | 23160198 | A/C |
| 4 | 23219817 | A/T |
| 4 | 23223331 | A/G |
| 4 | 23236611 | C/T |
| 4 | 23258962 | T/C |
| 4 | 23281687 | T/A |
| 4 | 23299677 | A/G |
| 4 | 23328149 | A/G |
| 4 | 23340692 | G/C |
| 4 | 23347138 | G/A |
| 4 | 23395768 | T/C |
| 4 | 23401989 | A/G |
| 4 | 23432390 | G/T |
| 4 | 23461212 | C/A |
| 4 | 23466794 | T/C |
| 4 | 23492120 | A/C |
| 4 | 23493346 | G/T |
| 4 | 23533350 | T/A |
| 4 | 23541290 | A/G |
| 4 | 23555114 | T/C |
| 4 | 23581111 | A/G |
| 4 | 23634871 | G/A |
| 4 | 23660817 | C/T |
| 4 | 23675434 | T/A |
| 4 | 23693779 | T/G |
| 4 | 23703131 | G/A |
| 4 | 23731492 | A/C |
| 4 | 23750038 | C/T |
| 4 | 23779785 | T/C |
| 4 | 23791615 | G/A |
| 4 | 23796827 | A/G |
| 4 | 23832992 | T/C |
| 4 | 23840571 | A/G |
| 4 | 23851241 | C/T |
| 4 | 23860249 | A/G |
| 4 | 23893347 | T/C |
| 4 | 23934736 | G/A |
| 4 | 23953911 | C/T |
| 4 | 23963701 | G/A |
| 4 | 23990238 | C/T |
| 4 | 24028844 | G/C |
| 4 | 24038329 | A/G |
| 4 | 24057353 | G/A |
| 4 | 24062954 | G/T |
| 4 | 24095593 | C/T |
| 4 | 24103241 | A/T |
| 4 | 24149544 | G/A |
| 4 | 24160508 | T/G |
| 4 | 24168886 | C/T |
| 4 | 24190833 | C/T |
| 4 | 24192831 | G/C |
| 4 | 24215259 | C/T |
| 4 | 24244636 | C/T |
| 4 | 24265773 | A/G |
| 4 | 24278225 | T/C |
| 4 | 24306528 | A/C |
| 4 | 24309098 | G/A |
| 4 | 24336706 | C/T |
| 4 | 24337791 | A/G |
| 4 | 24372334 | T/C |
| 4 | 24406042 | C/G |
| 4 | 24439714 | T/C |
| 4 | 24446845 | C/A |
| 4 | 24430277 | A/G |
| 4 | 24509995 | A/G |
| 4 | 24517614 | C/G |
| 4 | 24518922 | A/G |
| 4 | 24600271 | T/A |
| 4 | 24609197 | C/T |
| 4 | 24646559 | C/T |
| 4 | 24660302 | G/A |
| 4 | 24675247 | G/A |
| 4 | 24721145 | G/A |
| 4 | 24724762 | G/T |
| 4 | 24750934 | A/T |
| 4 | 24763180 | G/A |
| 4 | 24785534 | C/A |
| 4 | 24801678 | G/A |
| 4 | 24820577 | A/G |
| 4 | 24840470 | C/G |
| 4 | 24881318 | A/G |
| 4 | 24891835 | G/T |
| 4 | 24921476 | A/T |
| 4 | 24924489 | A/C |
| 4 | 24930013 | T/C |
| 4 | 24984649 | A/G |
| 4 | 25018782 | T/C |
| 4 | 25025270 | C/T |
| 4 | 25041102 | T/C |
| 4 | 25050291 | C/T |
| 4 | 25070570 | G/C |
| 4 | 25092931 | G/A |
| 4 | 25099098 | A/G |
| 4 | 25111140 | C/A |
| 4 | 25129828 | G/A |
| 4 | 25140576 | T/C |
| 4 | 25168924 | G/A |
| 4 | 25193371 | C/T |
| 4 | 25194390 | A/G |
| 4 | 25213285 | T/C |
| 4 | 25257618 | G/A |
| 4 | 25258618 | G/T |
| 4 | 25261927 | G/A |
| 4 | 25291803 | G/A |
| 4 | 25321670 | A/C |
| 4 | 25365707 | A/G |
| 4 | 25408673 | G/C |
| 4 | 25411608 | T/C |
| 4 | 25443486 | A/G |
| 4 | 25473651 | G/A |
| 4 | 25502887 | T/C |
| 4 | 25533884 | G/A |
| 4 | 25540578 | G/C |
| 4 | 25589322 | A/G |
| 4 | 25591386 | C/T |
| 4 | 25598590 | A/G |
| 4 | 25632513 | G/C |
| 4 | 25661708 | C/G |
| 4 | 25694300 | C/G |
| 4 | 25700005 | G/A |
| 4 | 25717611 | G/T |
| 4 | 25728970 | C/A |
| 4 | 25745190 | C/T |
| 4 | 25759247 | C/T |
| 4 | 25774694 | T/G |
| 4 | 25806166 | G/C |
| 4 | 25838347 | C/T |
| 4 | 25847804 | T/C |
| 4 | 25871267 | G/A |
| 4 | 25912616 | C/A |
| 4 | 25938968 | A/G |
| 4 | 25949103 | G/A |
| 4 | 25952190 | G/A |
| 4 | 25979775 | A/G |
| 4 | 26011876 | C/T |
| 4 | 26048027 | A/G |
| 4 | 26063594 | C/T |
| 4 | 26090558 | A/C |
| 4 | 26129542 | A/G |
| 4 | 26132454 | G/A |
| 4 | 26155067 | T/C |
| 4 | 26171851 | G/T |
| 4 | 26177447 | G/A |
| 4 | 26207806 | A/G |
| 4 | 26220011 | C/T |
| 4 | 26223092 | T/G |
| 4 | 26252007 | G/A |
| 4 | 26271195 | T/C |
| 4 | 26309199 | A/G |
| 4 | 26324333 | A/C |
| 4 | 26330746 | C/T |
| 4 | 26342635 | A/T |
| 4 | 26405930 | G/A |
| 4 | 26417573 | T/C |
| 4 | 26430068 | C/T |
| 4 | 26444399 | C/T |
| 4 | 26478703 | G/A |
| 4 | 26509052 | C/T |
| 4 | 26515759 | A/T |
| 4 | 26537540 | A/G |
| 4 | 26593925 | C/T |
| 4 | 26604304 | C/T |
| 4 | 26616518 | A/G |
| 4 | 26633190 | T/C |
| 4 | 26645877 | G/T |
| 4 | 26678810 | T/C |
| 4 | 26720628 | C/T |
| 4 | 26731752 | T/C |
| 4 | 26759331 | G/T |
| 4 | 26781524 | G/A |
| 4 | 26810678 | C/T |
| 4 | 26841492 | C/G |
| 4 | 26858827 | G/A |
| 4 | 26873272 | C/T |
| 4 | 26902736 | G/A |
| 4 | 26943805 | A/G |
| 4 | 26986711 | A/T |
| 4 | 27028202 | C/T |
| 4 | 27031143 | C/T |
| 4 | 27045566 | C/T |
| 4 | 27081856 | C/A |
| 4 | 27088388 | C/T |
| 4 | 27094726 | C/T |
| 4 | 27139374 | C/T |
| 4 | 27143901 | C/G |
| 4 | 27178152 | C/T |
| 4 | 27194835 | G/A |
| 4 | 27213380 | C/T |
| 4 | 27255981 | A/G |
| 4 | 27274893 | T/G |
| 4 | 27288689 | T/C |
| 4 | 27310174 | C/T |
| 4 | 27335231 | C/T |
| 4 | 27343284 | G/A |
| 4 | 27395356 | C/T |
| 4 | 27416588 | G/A |
| 4 | 27424003 | G/A |
| 4 | 27447478 | G/A |
| 4 | 27480078 | C/T |
| 4 | 27536192 | T/A |
| 4 | 27555883 | A/T |
| 4 | 27560484 | A/G |
| 4 | 27598386 | C/T |
| 4 | 27618091 | C/T |
| 4 | 27631151 | T/C |
| 4 | 27635930 | C/G |
| 4 | 27689934 | T/C |
| 4 | 27711658 | C/T |
| 4 | 27731690 | G/A |
| 4 | 27749878 | T/C |
| 4 | 27751643 | C/T |
| 4 | 27787020 | C/T |
| 4 | 27789553 | G/A |
| 4 | 27840337 | C/T |
| 4 | 27864449 | G/A |
| 4 | 27873504 | G/A |
| 4 | 27908010 | G/T |
| 4 | 27909300 | T/C |
| 4 | 27945205 | C/T |
| 4 | 27985108 | T/C |
| 4 | 27986172 | C/T |
| 4 | 28013181 | G/A |
| 4 | 28049014 | G/A |
| 4 | 28068831 | G/A |
| 4 | 28091794 | T/A |
| 4 | 28148278 | G/A |
| 4 | 28171249 | A/C |
| 4 | 28185037 | C/T |
| 4 | 28209266 | C/T |
| 4 | 28237162 | G/A |
| 4 | 28346188 | G/A |
| 4 | 28350764 | C/T |
| 4 | 28388692 | T/C |
| 4 | 28392888 | C/T |
| 4 | 28419323 | C/T |
| 4 | 28422946 | G/A |
| 4 | 28453265 | C/T |
| 4 | 28502814 | G/A |
| 4 | 28524069 | G/A |
| 4 | 28534774 | T/C |
| 4 | 28546689 | T/C |
| 4 | 28562180 | G/A |
| 4 | 28586397 | G/A |
| 4 | 28627043 | A/C |
| 4 | 28665306 | G/A |
| 4 | 28741611 | G/A |
| 4 | 28778741 | G/A |
| 4 | 28800149 | G/A |
| 4 | 28857830 | G/A |
| 4 | 28864066 | A/G |
| 4 | 28911449 | T/A |
| 4 | 28973062 | C/T |
| 4 | 29011315 | C/T |
| 4 | 29015874 | C/T |
| 4 | 29052179 | C/T |
| 4 | 29089820 | C/T |
| 4 | 29105197 | G/A |
| 4 | 29137104 | C/T |
| 4 | 29141910 | C/T |
| 4 | 29193375 | C/T |
| 4 | 29246506 | T/A |
| 4 | 29247892 | A/G |
| 4 | 29268750 | A/G |
| 4 | 29282146 | T/A |
| 4 | 29322713 | A/T |
| 4 | 29326314 | C/T |
| 4 | 29343587 | C/T |
| 4 | 29346370 | G/A |
| 4 | 29399063 | G/A |
| 4 | 29405856 | A/G |
| 4 | 29434412 | G/A |
| 4 | 29464875 | C/T |
| 4 | 29476737 | A/G |
| 4 | 29515252 | G/A |
| 4 | 29544497 | C/G |
| 4 | 29547357 | A/C |
| 4 | 29563160 | A/T |
| 4 | 29593450 | C/T |
| 4 | 29610979 | A/G |
| 4 | 29659979 | G/A |
| 4 | 29672101 | C/T |
| 4 | 29709869 | C/T |
| 4 | 29774960 | G/A |
| 4 | 29829720 | G/T |
| 4 | 29840392 | A/G |
| 4 | 29890856 | G/A |
| 4 | 29919098 | C/A |
| 4 | 29966558 | C/T |
| 4 | 29974376 | T/C |
| 4 | 29991068 | G/T |
| 4 | 30014133 | T/A |
| 4 | 30059738 | C/T |
| 4 | 30064006 | C/T |
| 4 | 30065556 | C/T |
| 4 | 30112443 | A/G |
| 4 | 30146228 | G/A |
| 4 | 30160162 | G/A |
| 4 | 30177015 | G/A |
| 4 | 30189142 | A/T |
| 4 | 30190827 | A/G |
| 4 | 30217004 | A/G |
| 4 | 30243384 | A/G |
| 4 | 30266951 | C/A |
| 4 | 30285810 | G/A |
| 4 | 30297972 | A/G |
| 4 | 30301199 | A/T |
| 4 | 30303832 | C/T |
| 4 | 30332074 | G/A |
| 4 | 30346671 | A/T |
| 4 | 30369398 | A/G |
| 4 | 30415001 | T/C |
| 4 | 30423794 | C/T |
| 4 | 30449171 | G/C |
| 4 | 30456640 | A/G |
| 4 | 30459985 | G/T |
| 4 | 30500477 | T/C |
| 4 | 30502753 | C/T |
| 4 | 30518619 | T/A |
| 4 | 30540116 | G/A |
| 4 | 30571902 | C/A |
| 4 | 30601947 | G/A |
| 4 | 30606100 | T/C |
| 4 | 30634480 | G/A |
| 4 | 30662492 | A/C |
| 4 | 30705513 | G/A |
| 4 | 30747347 | T/C |
| 4 | 30759081 | C/T |
| 4 | 30848744 | T/C |
| 4 | 30897559 | A/G |
| 4 | 30907681 | A/G |
| 4 | 30937339 | T/C |
| 4 | 30971508 | C/T |
| 4 | 30993345 | A/G |
| 4 | 31013624 | T/C |
| 4 | 31045738 | A/G |
| 4 | 31049510 | A/G |
| 4 | 31060225 | G/A |
| 4 | 31074186 | C/A |
| 4 | 31090691 | C/A |
| 4 | 31099076 | T/C |
| 4 | 31139925 | G/A |
| 4 | 31148119 | C/T |
| 4 | 31152204 | T/C |
| 4 | 31187023 | T/G |
| 4 | 31200268 | A/G |
| 4 | 31216355 | G/A |
| 4 | 31248178 | C/T |
| 4 | 31283739 | C/T |
| 4 | 31291167 | A/G |
| 4 | 31316508 | C/A |
| 4 | 31321888 | C/G |
| 4 | 31340203 | T/C |
| 4 | 31352988 | G/A |
| 4 | 31405874 | C/A |
| 4 | 31409699 | G/T |
| 4 | 31434977 | T/C |
| 4 | 31443832 | G/A |
| 4 | 31486461 | T/A |
| 4 | 31508927 | A/C |
| 4 | 31516243 | C/T |
| 4 | 31547065 | C/T |
| 4 | 31551263 | C/T |
| 4 | 31568765 | A/G |
| 4 | 31587979 | T/C |
| 4 | 31605649 | G/A |
| 4 | 31631470 | A/G |
| 4 | 31637087 | T/C |
| 4 | 31667885 | A/T |
| 4 | 31698416 | T/A |
| 4 | 31714495 | T/C |
| 4 | 31758464 | T/A |
| 4 | 31787442 | A/T |
| 4 | 31790174 | C/T |
| 4 | 31815413 | G/A |
| 4 | 31816536 | G/A |
| 4 | 31844709 | A/G |
| 4 | 31857517 | C/T |
| 4 | 31865627 | C/T |
| 4 | 31882031 | G/A |
| 4 | 31890457 | G/A |
| 4 | 31917074 | C/G |
| 4 | 31921891 | T/C |
| 4 | 31961645 | A/G |
| 4 | 31995264 | T/C |
| 4 | 31999205 | C/G |
| 4 | 32033660 | G/A |
| 4 | 32040520 | G/C |
| 4 | 32058906 | C/T |
| 4 | 32092717 | T/C |
| 4 | 32110280 | C/T |
| 4 | 32135893 | C/T |
| 4 | 32146303 | A/G |
| 4 | 32162125 | A/G |
| 4 | 32215656 | A/G |
| 4 | 32228371 | A/G |
| 4 | 32273727 | G/A |
| 4 | 32285226 | C/T |
| 4 | 32291010 | G/A |
| 4 | 32310094 | C/T |
| 4 | 32311604 | C/T |
| 4 | 32344174 | T/A |
| 4 | 32370885 | T/C |
| 4 | 32398974 | A/G |
| 4 | 32401203 | G/A |
| 4 | 32426710 | A/G |
| 4 | 32432777 | T/C |
| 4 | 32435017 | T/G |
| 4 | 32474963 | G/A |
| 4 | 32492757 | T/C |
| 4 | 32523554 | T/A |
| 4 | 32573217 | A/G |
| 4 | 32575935 | A/C |
| 4 | 32589638 | T/C |
| 4 | 32611462 | C/T |
| 4 | 32633413 | A/G |
| 4 | 32651656 | A/G |
| 4 | 32669350 | T/C |
| 4 | 32671136 | G/T |
| 4 | 32695502 | C/T |
| 4 | 32703421 | C/A |
| 4 | 32736554 | T/C |
| 4 | 32758670 | T/C |
| 4 | 32766470 | G/A |
| 4 | 32805600 | C/G |
| 4 | 32829451 | T/C |
| 4 | 32856426 | G/A |
| 4 | 32874099 | T/A |
| 4 | 32890725 | A/G |
| 4 | 32931063 | C/G |
| 4 | 32960994 | G/A |
| 4 | 32967613 | A/G |
| 4 | 32991624 | C/A |
| 4 | 32993705 | A/C |
| 4 | 33018879 | T/C |
| 4 | 33032164 | A/G |
| 4 | 33055017 | C/T |
| 4 | 33060181 | A/G |
| 4 | 33070523 | A/C |
| 4 | 33111770 | G/C |
| 4 | 33113002 | C/T |
| 4 | 33121999 | G/C |
| 4 | 33128202 | G/C |
| 4 | 33180217 | A/G |
| 4 | 33181986 | C/A |
| 4 | 33224980 | C/A |
| 4 | 33247392 | T/G |
| 4 | 33276718 | G/C |
| 4 | 33278454 | C/T |
| 4 | 33329134 | A/C |
| 4 | 33341315 | T/G |
| 4 | 33373523 | T/C |
| 4 | 33397044 | T/C |
| 4 | 33406248 | T/G |
| 4 | 33425356 | C/T |
| 4 | 33454278 | A/C |
| 4 | 33469244 | T/A |
| 4 | 33484831 | A/C |
| 4 | 33513852 | G/A |
| 4 | 33521630 | A/G |
| 4 | 33556211 | A/C |
| 4 | 33574243 | T/A |
| 4 | 33607748 | G/A |
| 4 | 33617811 | T/C |
| 4 | 33649517 | G/C |
| 4 | 33656950 | A/T |
| 4 | 33685133 | T/A |
| 4 | 33699375 | A/G |
| 4 | 33733384 | A/C |
| 4 | 33751576 | G/A |
| 4 | 33753046 | G/A |
| 4 | 33785758 | C/T |
| 4 | 33844836 | A/G |
| 4 | 33878690 | G/A |
| 4 | 33928226 | A/G |
| 4 | 33932346 | T/C |
| 4 | 33938040 | G/A |
| 4 | 33987538 | G/A |
| 4 | 34007895 | A/G |
| 4 | 34015864 | G/A |
| 4 | 34035042 | C/T |
| 4 | 34035043 | C/G |
| 4 | 34035044 | C/G |
| 4 | 34035045 | C/G |
| 4 | 34080057 | C/T |
| 4 | 34090397 | T/C |
| 4 | 34141276 | C/T |
| 4 | 34149648 | T/A |
| 4 | 34178773 | A/C |
| 4 | 34223504 | G/A |
| 4 | 34229111 | T/C |
| 4 | 34256802 | G/T |
| 4 | 34268566 | A/T |
| 4 | 34275296 | T/C |
| 4 | 34307825 | A/G |
| 4 | 34318550 | G/A |
| 4 | 34328533 | T/C |
| 4 | 34397633 | C/T |
| 4 | 34399193 | A/G |
| 4 | 34429195 | G/A |
| 4 | 34449885 | A/G |
| 4 | 34473447 | C/A |
| 4 | 34523507 | A/G |
| 4 | 34538978 | A/G |
| 4 | 34634428 | A/G |
| 4 | 34638507 | G/A |
| 4 | 34689730 | A/G |
| 4 | 34711240 | G/A |
| 4 | 34776669 | T/G |
| 4 | 34800248 | T/C |
| 4 | 34849599 | C/T |
| 4 | 34904065 | T/C |
| 4 | 34953571 | C/T |
| 4 | 34978043 | C/T |
| 4 | 35010576 | G/A |
| 4 | 35108395 | C/T |
| 4 | 35112709 | A/G |
| 4 | 35189999 | T/C |
| 4 | 35203692 | T/A |
| 4 | 35248115 | G/A |
| 4 | 35250089 | T/C |
| 4 | 35251290 | T/C |
| 4 | 35318130 | T/G |
| 4 | 35329325 | A/G |
| 4 | 35342341 | C/G |
| 4 | 35344751 | A/T |
| 4 | 35397548 | G/A |
| 4 | 35406900 | A/G |
| 4 | 35492870 | A/G |
| 4 | 35533754 | C/A |
| 4 | 35543950 | G/A |
| 4 | 35558687 | T/C |
| 4 | 35565958 | A/G |
| 4 | 35604257 | C/T |
| 4 | 35614194 | A/T |
| 4 | 35615940 | C/T |
| 4 | 35642665 | C/T |
| 4 | 35680746 | T/G |
| 4 | 35720571 | A/C |
| 4 | 35748309 | A/G |
| 4 | 35752477 | A/G |
| 4 | 35789804 | C/T |
| 4 | 35806451 | T/C |
| 4 | 35826110 | G/A |
| 4 | 35848286 | T/C |
| 4 | 35851174 | C/G |
| 4 | 35882795 | C/T |
| 4 | 35890050 | T/C |
| 4 | 35927447 | C/T |
| 4 | 35935916 | C/G |
| 4 | 35948007 | G/A |
| 4 | 35971929 | C/G |
| 4 | 35991725 | C/A |
| 4 | 36020283 | T/C |
| 4 | 36025448 | A/G |
| 4 | 36042710 | A/G |
| 4 | 36059138 | T/C |
| 4 | 36061551 | G/A |
| 4 | 36080773 | A/G |
| 4 | 36093124 | C/G |
| 4 | 36134584 | T/A |
| 4 | 36171190 | C/T |
| 4 | 36172192 | T/C |
| 4 | 36186705 | C/T |
| 4 | 36205241 | T/C |
| 4 | 36210409 | G/T |
| 4 | 36257663 | C/T |
| 4 | 36270924 | T/G |
| 4 | 36303246 | A/G |
| 4 | 36319086 | G/A |
| 4 | 36331435 | C/G |
| 4 | 36351033 | T/A |
| 4 | 36385446 | A/G |
| 4 | 36386939 | T/G |
| 4 | 36418177 | C/G |
| 4 | 36419512 | C/A |
| 4 | 36439814 | T/C |
| 4 | 36441478 | G/T |
| 4 | 36462894 | G/A |
| 4 | 36468982 | A/T |
| 4 | 36489620 | T/A |
| 4 | 36521612 | T/C |
| 4 | 36563671 | C/A |
| 4 | 36568918 | T/C |
| 4 | 36594511 | A/G |
| 4 | 36604515 | G/A |
| 4 | 36607606 | G/A |
| 4 | 36649542 | A/C |
| 4 | 36684327 | C/T |
| 4 | 36707584 | T/C |
| 4 | 36731462 | C/T |
| 4 | 36770937 | C/A |
| 4 | 36774820 | T/C |
| 4 | 36792686 | G/C |
| 4 | 36819477 | C/G |
| 4 | 36831125 | A/C |
| 4 | 36845326 | T/C |
| 4 | 36855677 | T/A |
| 4 | 36874319 | A/G |
| 4 | 36914339 | T/G |
| 4 | 36934763 | A/G |
| 4 | 36970734 | T/C |
| 4 | 36982746 | T/C |
| 4 | 36999370 | C/T |
| 4 | 37005550 | C/T |
| 4 | 37020073 | A/T |
| 4 | 37025367 | A/G |
| 4 | 37054154 | G/A |
| 4 | 37086909 | A/G |
| 4 | 37124385 | G/A |
| 4 | 37200622 | G/A |
| 4 | 37227888 | T/C |
| 4 | 37287531 | G/A |
| 4 | 37294067 | C/T |
| 4 | 37331595 | G/A |
| 4 | 37373339 | G/A |
| 4 | 37373340 | T/A |
| 4 | 37373341 | T/A |
| 4 | 37373342 | T/A |
| 4 | 37455712 | C/T |
| 4 | 37463275 | T/C |
| 4 | 37485224 | A/T |
| 4 | 37539416 | G/C |
| 4 | 37553971 | C/A |
| 4 | 37561465 | A/C |
| 4 | 37562829 | T/G |
| 4 | 37594175 | A/T |
| 4 | 37620256 | A/T |
| 4 | 37643154 | C/T |
| 4 | 37650480 | T/A |
| 4 | 37683806 | G/T |
| 4 | 37719982 | G/A |
| 4 | 37728347 | G/A |
| 4 | 37740442 | A/G |
| 4 | 37775901 | G/A |
| 4 | 37816393 | T/A |
| 4 | 37819821 | C/T |
| 4 | 37847611 | T/A |
| 4 | 37855056 | T/C |
| 4 | 37861787 | A/G |
| 4 | 37896783 | T/C |
| 4 | 37908406 | A/G |
| 4 | 37937377 | A/C |
| 4 | 37940557 | A/C |
| 4 | 37960822 | G/A |
| 4 | 37987516 | G/T |
| 4 | 38015319 | T/C |
| 4 | 38068639 | A/G |
| 4 | 38109150 | T/C |
| 4 | 38116876 | G/A |
| 4 | 38156318 | A/G |
| 4 | 38226127 | T/G |
| 4 | 38241591 | G/A |
| 4 | 38268457 | G/C |
| 4 | 38313877 | G/A |
| 4 | 38403014 | T/C |
| 4 | 38406827 | A/G |
| 4 | 38438752 | G/A |
| 4 | 38467735 | G/A |
| 4 | 38516944 | C/T |
| 4 | 38534169 | C/G |
| 4 | 38562558 | C/T |
| 4 | 38587554 | G/A |
| 4 | 38594878 | G/A |
| 4 | 38524522 | T/G |
| 4 | 28662380 | C/T |
| 4 | 38699243 | C/T |
| 4 | 38708482 | C/A |
| 4 | 38710949 | G/T |
| 4 | 38737018 | A/C |
| 4 | 38762646 | C/T |
| 4 | 38780952 | T/C |
| 4 | 38807393 | C/T |
| 4 | 38822971 | G/A |
| 4 | 38876187 | T/C |
| 4 | 38882429 | G/A |
| 4 | 38913187 | G/A |
| 4 | 38943396 | T/C |
| 4 | 38962321 | G/A |
| 4 | 38988516 | C/T |
| 4 | 39086350 | G/A |
| 4 | 39138674 | G/A |
| 4 | 39179589 | T/C |
| 4 | 39205499 | A/C |
| 4 | 39209198 | G/A |
| 4 | 39218932 | C/T |
| 4 | 39228214 | T/A |
| 4 | 39254677 | A/G |
| 4 | 39285607 | A/C |
| 4 | 39297800 | T/C |
| 4 | 39300126 | T/C |
| 4 | 39307314 | C/A |
| 4 | 39333441 | G/A |
| 4 | 39362293 | G/A |
| 4 | 39428568 | G/A |
| 4 | 39443461 | G/A |
| 4 | 39468871 | C/T |
| 4 | 39479489 | G/A |
| 4 | 39491129 | A/G |
| 4 | 39493577 | G/C |
| 4 | 39517416 | T/A |
| 4 | 39520870 | A/G |
| 4 | 39548781 | T/C |
| 4 | 39572269 | G/T |
| 4 | 39591612 | G/A |
| 4 | 39606617 | C/A |
| 4 | 39624521 | C/A |
| 4 | 39643799 | C/T |
| 4 | 39656371 | C/T |
| 4 | 39672312 | A/G |
| 4 | 39712264 | C/T |
| 4 | 39733474 | G/A |
| 4 | 39746809 | T/A |
| 4 | 39756156 | A/G |
| 4 | 39766731 | T/C |
| 4 | 39797428 | T/C |
| 4 | 39814039 | G/A |
| 4 | 39851197 | G/A |
| 4 | 39888022 | G/T |
| 4 | 39891411 | A/C |
| 4 | 39930189 | G/A |
| 4 | 39942975 | G/A |
| 4 | 39979309 | G/A |
| 4 | 40019533 | A/C |
| 4 | 40028136 | A/G |
| 4 | 40066109 | G/A |
| 4 | 40073853 | T/C |
| 4 | 40092048 | G/A |
| 4 | 40104428 | T/A |
| 4 | 40136797 | A/G |
| 4 | 40139946 | A/G |
| 4 | 40153038 | C/T |
| 4 | 40173964 | C/T |
| 4 | 40192518 | A/C |
| 4 | 40201453 | T/C |
| 4 | 40212899 | A/G |
| 4 | 40248373 | C/T |
| 4 | 40319350 | T/C |
| 4 | 40322077 | T/G |
| 4 | 40327263 | C/T |
| 4 | 40354124 | C/T |
| 4 | 40378039 | G/A |
| 4 | 40380865 | G/A |
| 4 | 40394301 | C/A |
| 4 | 40412943 | G/T |
| 4 | 40414294 | T/A |
| 4 | 40440007 | A/G |
| 4 | 40451236 | G/A |
| 4 | 40484135 | T/C |
| 4 | 40520684 | C/G |
| 4 | 40533820 | C/T |
| 4 | 40560108 | A/C |
| 4 | 40611148 | A/G |
| 4 | 40623730 | A/C |
| 4 | 40643862 | A/G |
| 4 | 40705641 | C/G |
| 4 | 40714873 | T/C |
| 4 | 40740518 | C/T |
| 4 | 40750963 | C/A |
| 4 | 40770746 | A/T |
| 4 | 40778767 | G/C |
| 4 | 40803552 | T/A |
| 4 | 40810968 | G/A |
| 4 | 40832654 | T/A |
| 4 | 40886813 | T/C |
| 4 | 40900348 | T/C |
| 4 | 40903446 | T/C |
| 4 | 40934681 | G/A |
| 4 | 40947031 | G/A |
| 4 | 40955281 | T/C |
| 4 | 40988466 | G/A |
| 4 | 40991912 | T/C |
| 4 | 41022831 | A/G |
| 4 | 41041322 | C/A |
| 4 | 41096995 | G/A |
| 4 | 41102378 | T/C |
| 4 | 41103644 | C/T |
| 4 | 41131123 | T/C |
| 4 | 41143278 | A/G |
| 4 | 41184203 | G/C |
| 4 | 41268840 | G/A |
| 4 | 41305442 | C/G |
| 4 | 41306466 | T/C |
| 4 | 41333531 | A/G |
| 4 | 41344776 | T/C |
| 4 | 41351142 | G/A |
| 4 | 41376845 | C/T |
| 4 | 41405169 | C/T |
| 4 | 41427506 | A/G |
| 4 | 41436903 | T/C |
| 4 | 41465544 | C/T |
| 4 | 41467537 | A/G |
| 4 | 41511662 | T/C |
| 4 | 41556449 | A/G |
| 4 | 41579092 | A/T |
| 4 | 41599955 | C/T |
| 4 | 41600247 | G/A |
| 4 | 41605644 | A/G |
| 4 | 41629443 | G/A |
| 4 | 41656047 | A/G |
| 4 | 41666765 | C/T |
| 4 | 41718785 | G/A |
| 4 | 41734839 | T/C |
| 4 | 41779435 | A/G |
| 4 | 41798950 | C/G |
| 4 | 41817288 | A/G |
| 4 | 41838620 | G/A |
| 4 | 41850848 | T/C |
| 4 | 41855037 | C/T |
| 4 | 41880595 | G/T |
| 4 | 41895287 | C/T |
| 4 | 41924506 | C/T |
| 4 | 41948295 | A/T |
| 4 | 41976290 | G/A |
| 4 | 42027895 | T/C |
| 4 | 42049442 | C/G |
| 4 | 42053477 | A/G |
| 4 | 42065209 | A/G |
| 4 | 42066888 | G/C |
| 4 | 42171143 | C/T |
| 4 | 42195533 | C/T |
| 4 | 42235204 | C/G |
| 4 | 42239010 | G/A |
| 4 | 44240255 | G/A |
| 4 | 42283244 | C/T |
| 4 | 42307095 | A/G |
| 4 | 42335715 | G/A |
| 4 | 42372985 | C/T |
| 4 | 42378366 | A/G |
| 4 | 42390362 | C/T |
| 4 | 42446893 | T/C |
| 4 | 42450091 | C/G |
| 4 | 42494114 | A/G |
| 4 | 42508276 | G/A |
| 4 | 42516962 | C/G |
| 4 | 42520394 | C/T |
| 4 | 42541052 | T/A |
| 4 | 42563682 | C/A |
| 4 | 42573204 | T/C |
| 4 | 42618464 | C/T |
| 4 | 42632838 | C/T |
| 4 | 42652814 | A/G |
| 4 | 42660043 | G/T |
| 4 | 42667987 | T/G |
| 4 | 42724711 | A/G |
| 4 | 42740726 | C/T |
| 4 | 42779826 | G/A |
| 4 | 42783585 | T/C |
| 4 | 42829529 | C/A |
| 4 | 42844619 | T/G |
| 4 | 42870554 | A/T |
| 4 | 42930149 | C/T |
| 4 | 42935439 | C/T |
| 4 | 42962953 | C/T |
| 4 | 43004964 | T/A |
| 4 | 43024545 | G/T |
| 4 | 43056490 | A/G |
| 4 | 43093246 | C/G |
| 4 | 43102870 | C/T |
| 4 | 43113943 | A/G |
| 4 | 43145328 | G/A |
| 4 | 43160537 | C/T |
| 4 | 43188351 | T/C |
| 4 | 43188697 | C/G |
| 4 | 43203951 | A/G |
| 4 | 43215614 | G/A |
| 4 | 43251824 | C/A |
| 4 | 43266384 | C/G |
| 4 | 43283569 | A/C |
| 4 | 43298968 | G/T |
| 4 | 43304976 | A/G |
| 4 | 43361130 | G/A |
| 4 | 43365428 | A/C |
| 4 | 43398755 | T/C |
| 4 | 43415922 | C/T |
| 4 | 43431576 | G/A |
| 4 | 43465548 | T/C |
| 4 | 43466935 | A/G |
| 4 | 43498577 | G/C |
| 4 | 43503239 | G/T |
| 4 | 43532778 | C/T |
| 4 | 43543558 | C/T |
| 4 | 43571830 | T/C |
| 4 | 43587785 | C/T |
| 4 | 43616320 | C/T |
| 4 | 43619171 | T/C |
| 4 | 43621446 | G/T |
| 4 | 43652079 | T/C |
| 4 | 43667961 | C/A |
| 4 | 43706299 | G/A |
| 4 | 43715599 | C/T |
| 4 | 43716850 | A/G |
| 4 | 43745907 | T/G |
| 4 | 43768334 | G/T |
| 4 | 43788546 | T/G |
| 4 | 43790071 | C/A |
| 4 | 43806617 | C/A |
| 4 | 43825137 | C/G |
| 4 | 43836177 | C/T |
| 4 | 43857655 | A/G |
| 4 | 43882472 | G/A |
| 4 | 43888723 | A/G |
| 4 | 43907960 | C/G |
| 4 | 43910252 | T/C |
| 4 | 43930712 | T/A |
| 4 | 43945469 | G/A |
| 4 | 43952366 | T/C |
| 4 | 43957129 | T/C |
| 4 | 43986305 | C/A |
| 4 | 44002121 | G/C |
| 5 | 8985 | C/T |
| 5 | 14319 | G/A |
| 5 | 36408 | C/T |
| 5 | 60074 | A/T |
| 5 | 96066 | G/A |
| 5 | 120897 | G/A |
| 5 | 128939 | C/G |
| 5 | 164517 | G/A |
| 5 | 206078 | C/A |
| 5 | 210641 | C/A |
| 5 | 243350 | C/T |
| 5 | 253467 | T/A |
| 5 | 276643 | T/A |
| 5 | 312944 | G/A |
| 5 | 313057 | A/C |
| 5 | 313788 | A/T |
| 5 | 314372 | A/T |
| 5 | 333749 | A/G |
| 5 | 374874 | G/A |
| 5 | 397226 | C/T |
| 5 | 399734 | A/T |
| 5 | 430474 | C/G |
| 5 | 454179 | C/T |
| 5 | 477135 | C/G |
| 5 | 500142 | T/A |
| 5 | 503520 | G/C |
| 5 | 511727 | A/G |
| 5 | 539475 | C/T |
| 5 | 549205 | C/T |
| 5 | 578534 | A/G |
| 5 | 593854 | T/C |
| 5 | 604845 | T/C |
| 5 | 618030 | T/C |
| 5 | 643234 | T/C |
| 5 | 651668 | T/A |
| 5 | 660756 | C/T |
| 5 | 715340 | T/C |
| 5 | 719841 | T/A |
| 5 | 722840 | G/C |
| 5 | 726610 | G/A |
| 5 | 750316 | A/T |
| 5 | 777286 | G/T |
| 5 | 788772 | T/A |
| 5 | 796836 | G/A |
| 5 | 830965 | T/C |
| 5 | 841504 | C/T |
| 5 | 855917 | C/T |
| 5 | 876731 | T/G |
| 5 | 877743 | C/A |
| 5 | 900231 | C/A |
| 5 | 901364 | A/G |
| 5 | 938885 | G/A |
| 5 | 960680 | C/A |
| 5 | 1010321 | C/T |
| 5 | 1027467 | C/T |
| 5 | 1077142 | G/A |
| 5 | 1080365 | T/G |
| 5 | 1083351 | A/G |
| 5 | 1111105 | T/C |
| 5 | 1139692 | T/G |
| 5 | 1142132 | C/G |
| 5 | 1164843 | A/C |
| 5 | 1170413 | A/T |
| 5 | 1196728 | T/C |
| 5 | 1203920 | C/T |
| 5 | 1211259 | C/T |
| 5 | 1235785 | C/A |
| 5 | 1268122 | G/A |
| 5 | 1289832 | C/T |
| 5 | 1309418 | G/A |
| 5 | 1330406 | C/T |
| 5 | 1351971 | C/T |
| 5 | 1387947 | A/G |
| 5 | 1398369 | G/T |
| 5 | 1437091 | A/G |
| 5 | 1437740 | A/G |
| 5 | 1438470 | G/T |
| 5 | 1438988 | A/C |
| 5 | 1439103 | C/T |
| 5 | 1445871 | G/C |
| 5 | 1451331 | A/G |
| 5 | 1472696 | G/A |
| 5 | 1499035 | A/C |
| 5 | 1503883 | A/T |
| 5 | 1532318 | G/A |
| 5 | 1537490 | C/A |
| 5 | 1563352 | T/C |
| 5 | 1592629 | G/T |
| 5 | 1614408 | A/T |
| 5 | 1631020 | C/T |
| 5 | 1642719 | G/C |
| 5 | 1650879 | G/C |
| 5 | 1691794 | T/A |
| 5 | 1694204 | G/A |
| 5 | 1714286 | G/A |
| 5 | 1751225 | G/A |
| 5 | 1761026 | G/A |
| 5 | 1776145 | T/C |
| 5 | 1791302 | C/G |
| 5 | 1801473 | G/C |
| 5 | 1845236 | T/A |
| 5 | 1855487 | G/A |
| 5 | 1862982 | T/A |
| 5 | 1876217 | C/T |
| 5 | 1908195 | G/A |
| 5 | 1942349 | A/G |
| 5 | 1949494 | C/T |
| 5 | 1963048 | G/T |
| 5 | 1966710 | G/A |
| 5 | 1984367 | G/C |
| 5 | 1985692 | A/C |
| 5 | 2026836 | G/A |
| 5 | 2039771 | A/G |
| 5 | 2040988 | G/C |
| 5 | 2073364 | T/C |
| 5 | 2113731 | C/T |
| 5 | 2151192 | G/A |
| 5 | 2157242 | A/T |
| 5 | 2174341 | A/C |
| 5 | 2185748 | C/G |
| 5 | 2206946 | G/A |
| 5 | 2212873 | A/T |
| 5 | 2224049 | A/G |
| 5 | 2238731 | G/A |
| 5 | 2268212 | C/T |
| 5 | 2281484 | A/T |
| 5 | 2329345 | A/G |
| 5 | 2348972 | G/A |
| 5 | 2394497 | G/C |
| 5 | 2402963 | C/A |
| 5 | 2428641 | A/T |
| 5 | 2432270 | G/T |
| 5 | 2451789 | G/A |
| 5 | 2468063 | A/C |
| 5 | 2486090 | T/A |
| 5 | 2499204 | A/C |
| 5 | 2507111 | A/G |
| 5 | 2507112 | A/G |
| 5 | 2507113 | A/G |
| 5 | 2507114 | A/G |
| 5 | 2577019 | C/G |
| 5 | 2588907 | G/A |
| 5 | 2610473 | G/A |
| 5 | 2681995 | G/C |
| 5 | 2688146 | G/A |
| 5 | 2702149 | C/T |
| 5 | 2714474 | T/C |
| 5 | 2731984 | G/C |
| 5 | 2745498 | A/G |
| 5 | 2781782 | C/T |
| 5 | 2795746 | C/T |
| 5 | 2822007 | A/G |
| 5 | 2850893 | A/T |
| 5 | 2866396 | C/T |
| 5 | 2892192 | G/A |
| 5 | 2913452 | C/T |
| 5 | 2928251 | G/A |
| 5 | 2946321 | A/G |
| 5 | 2985710 | G/C |
| 5 | 2992077 | G/A |
| 5 | 3018760 | T/C |
| 5 | 3021818 | G/A |
| 5 | 3053984 | G/A |
| 5 | 3117868 | G/A |
| 5 | 3129294 | C/T |
| 5 | 3135057 | G/T |
| 5 | 3180899 | A/T |
| 5 | 3189703 | C/T |
| 5 | 3235189 | C/A |
| 5 | 3240830 | G/A |
| 5 | 3295377 | G/A |
| 5 | 3303610 | G/A |
| 5 | 3323042 | C/T |
| 5 | 3335488 | C/A |
| 5 | 3349034 | C/T |
| 5 | 3361257 | C/T |
| 5 | 3390664 | C/T |
| 5 | 3394527 | G/A |
| 5 | 3422617 | A/G |
| 5 | 3451689 | A/T |
| 5 | 3476958 | C/G |
| 5 | 3481527 | C/A |
| 5 | 3502905 | C/G |
| 5 | 3510102 | A/T |
| 5 | 3522689 | T/A |
| 5 | 3541128 | A/C |
| 5 | 3542282 | A/T |
| 5 | 3573709 | G/A |
| 5 | 3576354 | G/T |
| 5 | 3626703 | G/A |
| 5 | 3628295 | C/T |
| 5 | 3634991 | C/T |
| 5 | 3664662 | G/A |
| 5 | 3670072 | G/T |
| 5 | 3691060 | A/C |
| 5 | 3705146 | A/T |
| 5 | 3749735 | C/T |
| 5 | 3762391 | T/C |
| 5 | 3776871 | A/T |
| 5 | 3788664 | C/G |
| 5 | 3801684 | C/T |
| 5 | 3815535 | G/A |
| 5 | 3832033 | G/A |
| 5 | 3843417 | C/G |
| 5 | 3863266 | T/A |
| 5 | 3880581 | T/C |
| 5 | 3891690 | G/T |
| 5 | 3915708 | T/C |
| 5 | 3917930 | G/A |
| 5 | 3939650 | G/A |
| 5 | 3973581 | A/G |
| 5 | 3980453 | T/A |
| 5 | 4006935 | T/G |
| 5 | 4029764 | T/A |
| 5 | 4032935 | G/A |
| 5 | 4064727 | G/C |
| 5 | 4085672 | C/G |
| 5 | 4090818 | G/A |
| 5 | 4117840 | G/A |
| 5 | 4162798 | A/G |
| 5 | 4180695 | T/C |
| 5 | 4188287 | A/G |
| 5 | 4226591 | C/T |
| 5 | 4227643 | T/C |
| 5 | 4230409 | T/G |
| 5 | 4267101 | A/G |
| 5 | 4269794 | G/A |
| 5 | 4291160 | C/A |
| 5 | 4321068 | T/C |
| 5 | 4368914 | A/G |
| 5 | 4372311 | A/T |
| 5 | 4392401 | T/C |
| 5 | 4405742 | A/G |
| 5 | 4425503 | A/C |
| 5 | 4436739 | C/T |
| 5 | 4448685 | G/A |
| 5 | 4474060 | T/C |
| 5 | 4502597 | C/G |
| 5 | 4521715 | G/T |
| 5 | 4530574 | G/A |
| 5 | 4565996 | A/G |
| 5 | 4568784 | C/T |
| 5 | 4617045 | G/A |
| 5 | 4646480 | A/G |
| 5 | 4658418 | G/A |
| 5 | 4669547 | A/G |
| 5 | 4703196 | C/T |
| 5 | 4723164 | A/G |
| 5 | 4725307 | T/C |
| 5 | 4748688 | G/A |
| 5 | 4754369 | G/A |
| 5 | 4770920 | T/C |
| 5 | 4822460 | A/C |
| 5 | 4830563 | A/G |
| 5 | 4858592 | G/C |
| 5 | 4878365 | G/A |
| 5 | 4880342 | C/T |
| 5 | 4890857 | C/T |
| 5 | 4904553 | G/T |
| 5 | 4929922 | C/T |
| 5 | 4975210 | T/C |
| 5 | 4991374 | T/C |
| 5 | 5012821 | T/C |
| 5 | 5031356 | A/G |
| 5 | 5040881 | G/A |
| 5 | 5071306 | G/A |
| 5 | 5081069 | G/A |
| 5 | 5105356 | G/A |
| 5 | 5123344 | C/T |
| 5 | 5151280 | A/G |
| 5 | 5157934 | C/T |
| 5 | 5165882 | C/A |
| 5 | 5203283 | C/T |
| 5 | 5224664 | C/T |
| 5 | 5247139 | C/G |
| 5 | 5254973 | G/A |
| 5 | 5261406 | T/C |
| 5 | 5300151 | G/A |
| 5 | 5318615 | T/C |
| 5 | 5356816 | C/T |
| 5 | 5357831 | C/T |
| 5 | 5393777 | C/T |
| 5 | 5399251 | C/G |
| 5 | 5426569 | G/A |
| 5 | 5446251 | C/T |
| 5 | 5452759 | G/A |
| 5 | 5460596 | T/C |
| 5 | 5499272 | A/G |
| 5 | 5507971 | C/T |
| 5 | 5520960 | A/G |
| 5 | 5524712 | C/T |
| 5 | 5550414 | G/A |
| 5 | 5551523 | C/T |
| 5 | 5586939 | T/G |
| 5 | 5602144 | T/C |
| 5 | 5620594 | G/A |
| 5 | 5636084 | A/G |
| 5 | 5648555 | G/A |
| 5 | 5650904 | G/A |
| 5 | 5674459 | G/T |
| 5 | 5678289 | T/A |
| 5 | 5719250 | T/C |
| 5 | 5722512 | A/T |
| 5 | 5734965 | C/T |
| 5 | 5785096 | A/T |
| 5 | 5816591 | A/T |
| 5 | 5818801 | G/A |
| 5 | 5821600 | C/T |
| 5 | 5835797 | T/C |
| 5 | 5850370 | C/A |
| 5 | 5874628 | T/C |
| 5 | 5890820 | C/G |
| 5 | 5909675 | C/T |
| 5 | 5912572 | T/A |
| 5 | 5922934 | A/T |
| 5 | 5960749 | C/T |
| 5 | 5968979 | A/G |
| 5 | 5999413 | T/G |
| 5 | 6020055 | A/G |
| 5 | 6030417 | A/G |
| 5 | 6049900 | A/G |
| 5 | 6062770 | C/T |
| 5 | 6073875 | C/A |
| 5 | 6101660 | T/C |
| 5 | 6104825 | C/G |
| 5 | 6120544 | T/C |
| 5 | 6138564 | C/T |
| 5 | 6174573 | T/C |
| 5 | 6197208 | A/T |
| 5 | 6200281 | G/A |
| 5 | 6220926 | G/T |
| 5 | 6255844 | T/A |
| 5 | 6258717 | G/C |
| 5 | 6285994 | T/A |
| 5 | 6295431 | G/A |
| 5 | 6307642 | G/A |
| 5 | 6346135 | G/A |
| 5 | 6371147 | A/G |
| 5 | 6374922 | C/T |
| 5 | 6391452 | G/C |
| 5 | 6413791 | G/C |
| 5 | 6428951 | A/T |
| 5 | 6443767 | G/A |
| 5 | 6478787 | T/C |
| 5 | 6482130 | T/A |
| 5 | 6489737 | A/G |
| 5 | 6525157 | G/A |
| 5 | 6539437 | C/T |
| 5 | 6540682 | A/G |
| 5 | 6593162 | T/G |
| 5 | 6595933 | C/A |
| 5 | 6609011 | C/G |
| 5 | 6615224 | T/G |
| 5 | 6643917 | G/T |
| 5 | 6657461 | A/G |
| 5 | 6682488 | A/G |
| 5 | 6696269 | C/T |
| 5 | 6701262 | C/G |
| 5 | 6723640 | T/C |
| 5 | 6774237 | G/A |
| 5 | 6777752 | T/G |
| 5 | 6801766 | A/G |
| 5 | 6842193 | C/G |
| 5 | 6843754 | A/T |
| 5 | 6895199 | A/G |
| 5 | 6913474 | C/A |
| 5 | 6914748 | C/T |
| 5 | 6961491 | A/C |
| 5 | 6989027 | A/G |
| 5 | 6991096 | T/C |
| 5 | 7032876 | C/T |
| 5 | 7035214 | G/C |
| 5 | 7064300 | T/G |
| 5 | 7069316 | C/G |
| 5 | 7101541 | T/C |
| 5 | 7104491 | T/C |
| 5 | 7110035 | A/T |
| 5 | 7112946 | A/T |
| 5 | 7140133 | G/C |
| 5 | 7142585 | C/T |
| 5 | 7220053 | G/C |
| 5 | 7224088 | G/T |
| 5 | 7237631 | T/A |
| 5 | 7274057 | A/T |
| 5 | 7277645 | C/T |
| 5 | 7292449 | G/A |
| 5 | 7310512 | A/G |
| 5 | 7339167 | A/G |
| 5 | 7350699 | A/C |
| 5 | 7380432 | A/C |
| 5 | 7382084 | C/G |
| 5 | 7422244 | A/T |
| 5 | 7426365 | C/T |
| 5 | 7448676 | G/A |
| 5 | 7493857 | T/G |
| 5 | 7497570 | A/T |
| 5 | 7520731 | G/A |
| 5 | 7546089 | A/T |
| 5 | 7561117 | C/G |
| 5 | 7587768 | G/A |
| 5 | 7613273 | A/G/ |
| 5 | 7618788 | C/G |
| 5 | 7620610 | C/T |
| 5 | 7625714 | A/G |
| 5 | 7669906 | T/G |
| 5 | 7670938 | C/G |
| 5 | 7706175 | A/G |
| 5 | 7713945 | C/T |
| 5 | 7720283 | T/A |
| 5 | 7741371 | T/C |
| 5 | 7754354 | A/G |
| 5 | 7771215 | A/G |
| 5 | 7825837 | T/G |
| 5 | 7828628 | C/A |
| 5 | 7841612 | A/G |
| 5 | 7873716 | T/C |
| 5 | 7880624 | T/C |
| 5 | 7892879 | A/G |
| 5 | 7903218 | G/A |
| 5 | 7935483 | G/C |
| 5 | 7936548 | T/C |
| 5 | 7952142 | C/T |
| 5 | 7965094 | A/C |
| 5 | 8003389 | G/C |
| 5 | 8005811 | T/C |
| 5 | 8017469 | G/A |
| 5 | 8025487 | T/G |
| 5 | 8045648 | T/C |
| 5 | 8060308 | C/T |
| 5 | 8073599 | T/C |
| 5 | 8081382 | G/C |
| 5 | 8124600 | A/G |
| 5 | 8126045 | A/G |
| 5 | 8130131 | T/C |
| 5 | 8132167 | G/C |
| 5 | 8198897 | T/G |
| 5 | 8237345 | C/A |
| 5 | 8257327 | G/T |
| 5 | 8258653 | T/A |
| 5 | 8258654 | G/A |
| 5 | 8311502 | C/T |
| 5 | 8345635 | T/C |
| 5 | 8351664 | A/G |
| 5 | 8410112 | T/C |
| 5 | 8423562 | C/T |
| 5 | 8438525 | T/A |
| 5 | 8468357 | G/A |
| 5 | 8480496 | C/T |
| 5 | 8497083 | A/T |
| 5 | 8504256 | A/G |
| 5 | 8539347 | A/G |
| 5 | 8554885 | T/A |
| 5 | 8570508 | G/A |
| 5 | 8586401 | A/G |
| 5 | 8587715 | C/T |
| 5 | 8634090 | A/G |
| 5 | 8635380 | A/G |
| 5 | 8651761 | T/C |
| 5 | 8652953 | C/G |
| 5 | 8699290 | C/T |
| 5 | 8720531 | G/A |
| 5 | 8752715 | C/A |
| 5 | 8755676 | C/G |
| 5 | 8771977 | A/C |
| 5 | 8779637 | G/C |
| 5 | 8814069 | G/A |
| 5 | 8819709 | T/C |
| 5 | 8819710 | A/G |
| 5 | 8878865 | G/T |
| 5 | 8894650 | A/C |
| 5 | 8907708 | C/T |
| 5 | 8922921 | A/C |
| 5 | 8932153 | A/C |
| 5 | 8941517 | T/C |
| 5 | 8948261 | G/C |
| 5 | 8971433 | T/C |
| 5 | 8973194 | C/T |
| 5 | 9025450 | G/C |
| 5 | 9026803 | A/T |
| 5 | 9040876 | G/A |
| 5 | 9052201 | G/C |
| 5 | 9069347 | C/T |
| 5 | 9091667 | A/G |
| 5 | 9106422 | G/A |
| 5 | 9140740 | T/G |
| 5 | 9160118 | T/C |
| 5 | 9172497 | C/G |
| 5 | 9199108 | G/C |
| 5 | 9262993 | G/A |
| 5 | 9284611 | C/G |
| 5 | 9285716 | T/A |
| 5 | 9312278 | G/T |
| 5 | 9344752 | G/A |
| 5 | 9385252 | T/C |
| 5 | 9419636 | C/T |
| 5 | 9423028 | T/G |
| 5 | 9426209 | C/T |
| 5 | 9464495 | G/A |
| 5 | 9489233 | G/C |
| 5 | 9517444 | C/A |
| 5 | 9550928 | A/G |
| 5 | 9586909 | T/A |
| 5 | 9589694 | T/C |
| 5 | 9615327 | C/T |
| 5 | 9655065 | G/C |
| 5 | 9665370 | A/C |
| 5 | 9688345 | A/G |
| 5 | 9696451 | G/A |
| 5 | 9722649 | A/C |
| 5 | 9772509 | G/A |
| 5 | 9802573 | T/A |
| 5 | 9807623 | C/T |
| 5 | 9819840 | T/A |
| 5 | 9820990 | C/T |
| 5 | 9841085 | T/C |
| 5 | 9844473 | G/A |
| 5 | 9871340 | T/C |
| 5 | 9894367 | G/A |
| 5 | 9904305 | C/T |
| 5 | 9941946 | T/A |
| 5 | 9985831 | C/T |
| 5 | 9998669 | T/C |
| 5 | 10011835 | A/C |
| 5 | 10037562 | C/A |
| 5 | 10069213 | G/A |
| 5 | 10072340 | G/C |
| 5 | 10111114 | C/T |
| 5 | 10114200 | G/T |
| 5 | 10156465 | A/T |
| 5 | 10196133 | A/G |
| 5 | 10197171 | T/A |
| 5 | 10207767 | A/C |
| 5 | 10234133 | G/C |
| 5 | 10245517 | C/G |
| 5 | 10266123 | G/C |
| 5 | 10267780 | G/T |
| 5 | 10297230 | G/T |
| 5 | 10346432 | T/A |
| 5 | 10349414 | A/C |
| 5 | 10362555 | T/C |
| 5 | 10366955 | C/T |
| 5 | 10400088 | C/T |
| 5 | 10410319 | G/A |
| 5 | 10414669 | T/C |
| 5 | 10444500 | C/T |
| 5 | 10463080 | T/G |
| 5 | 10473806 | G/A |
| 5 | 10480326 | G/C |
| 5 | 10524812 | G/A |
| 5 | 10544025 | C/G |
| 5 | 10547957 | T/C |
| 5 | 10561847 | A/G |
| 5 | 10568544 | C/T |
| 5 | 10630844 | C/T |
| 5 | 10631845 | G/A |
| 5 | 10655631 | A/C |
| 5 | 10685099 | G/A |
| 5 | 10685100 | T/C |
| 5 | 10685101 | T/C |
| 5 | 10685102 | T/C |
| 5 | 10745713 | T/C |
| 5 | 10770849 | A/C |
| 5 | 10814486 | A/G |
| 5 | 10817126 | C/A |
| 5 | 10834337 | G/A |
| 5 | 10838048 | G/A |
| 5 | 10880671 | G/A |
| 5 | 10904596 | T/C |
| 5 | 10909677 | G/A |
| 5 | 10920049 | G/A |
| 5 | 10955242 | A/G |
| 5 | 10964995 | G/T |
| 5 | 10987520 | C/A |
| 5 | 11017540 | T/C |
| 5 | 11033703 | G/T |
| 5 | 11047390 | G/A |
| 5 | 11090498 | C/A |
| 5 | 11090952 | C/T |
| 5 | 11091024 | G/A |
| 5 | 11091742 | A/G |
| 5 | 11124094 | C/G |
| 5 | 11145496 | G/A |
| 5 | 11150859 | T/C |
| 5 | 11161086 | T/G |
| 5 | 11163374 | A/G |
| 5 | 11190536 | A/G |
| 5 | 11232055 | G/A |
| 5 | 11238579 | C/T |
| 5 | 11270896 | A/C |
| 5 | 11276279 | C/T |
| 5 | 13303893 | G/A |
| 5 | 11305915 | A/G |
| 5 | 11311924 | T/C |
| 5 | 11362079 | A/T |
| 5 | 11370903 | G/A |
| 5 | 11399992 | T/C |
| 5 | 11402470 | G/A |
| 5 | 11410847 | G/A |
| 5 | 11436864 | A/G |
| 5 | 11443454 | A/C |
| 5 | 11475559 | A/T |
| 5 | 11482317 | C/T |
| 5 | 11514416 | C/T |
| 5 | 11538437 | G/A |
| 5 | 11563254 | A/G |
| 5 | 11569712 | G/A |
| 5 | 11581188 | T/C |
| 5 | 11590592 | C/T |
| 5 | 11613743 | G/A |
| 5 | 11617952 | G/A |
| 5 | 11648128 | T/G |
| 5 | 11658218 | T/G |
| 5 | 11700341 | C/T |
| 5 | 11737394 | C/T |
| 5 | 11789249 | T/C |
| 5 | 11797956 | G/A |
| 5 | 11820062 | A/G |
| 5 | 11836796 | G/A |
| 5 | 11898785 | G/C |
| 5 | 11924548 | G/T |
| 5 | 11933250 | C/T |
| 5 | 11957116 | C/G |
| 5 | 12008961 | A/G |
| 5 | 12039936 | A/G |
| 5 | 12062003 | G/A |
| 5 | 12070314 | C/G |
| 5 | 12106396 | C/T |
| 5 | 12132352 | G/A |
| 5 | 12138607 | A/G |
| 5 | 12169282 | C/T |
| 5 | 12179699 | A/T |
| 5 | 12181938 | T/C |
| 5 | 12203604 | G/A |
| 5 | 12212994 | T/C |
| 5 | 12247230 | G/A |
| 5 | 12273396 | T/C |
| 5 | 12303005 | C/T |
| 5 | 12325421 | C/T |
| 5 | 12339217 | A/G |
| 5 | 12356272 | A/G |
| 5 | 12361752 | G/A |
| 5 | 12400526 | G/T |
| 5 | 12414202 | A/G |
| 5 | 12438089 | G/T |
| 5 | 12439917 | G/T |
| 5 | 12457458 | C/T |
| 5 | 12459346 | C/T |
| 5 | 12521826 | A/G |
| 5 | 12548610 | G/T |
| 5 | 12551710 | A/T |
| 5 | 12581385 | A/C |
| 5 | 12603510 | A/G |
| 5 | 12648627 | A/G |
| 5 | 12669091 | G/A |
| 5 | 12678632 | G/A |
| 5 | 12701796 | C/A |
| 5 | 12709931 | C/T |
| 5 | 12724897 | T/G |
| 5 | 12741678 | A/T |
| 5 | 12778874 | C/T |
| 5 | 12782052 | C/T |
| 5 | 12786166 | C/T |
| 5 | 12821270 | T/G |
| 5 | 12822500 | A/G |
| 5 | 12859096 | G/A |
| 5 | 12866398 | G/A |
| 5 | 12873914 | A/G |
| 5 | 12883162 | T/C |
| 5 | 12922434 | C/T |
| 5 | 12938074 | A/T |
| 5 | 12960314 | G/A |
| 5 | 13002497 | T/C |
| 5 | 13007251 | G/A |
| 5 | 13040177 | C/T |
| 5 | 13061274 | G/A |
| 5 | 13076909 | A/G |
| 5 | 13102326 | C/T |
| 5 | 13107165 | T/C |
| 5 | 13130279 | G/T |
| 5 | 13152152 | G/A |
| 5 | 13170697 | A/G |
| 5 | 13196736 | A/C |
| 5 | 13205061 | A/G |
| 5 | 13205062 | C/T |
| 5 | 13205063 | T/C |
| 5 | 13276130 | T/G |
| 5 | 13287875 | C/T |
| 5 | 13290552 | G/T |
| 5 | 13324652 | C/T |
| 5 | 13349631 | G/C |
| 5 | 13370057 | A/G |
| 5 | 13395881 | C/T |
| 5 | 13400888 | C/T |
| 5 | 13417858 | T/C |
| 5 | 13429497 | T/G |
| 5 | 13440376 | T/C |
| 5 | 13455741 | A/G |
| 5 | 13489849 | A/G |
| 5 | 13557585 | C/G |
| 5 | 13568378 | G/A |
| 5 | 13583443 | G/A |
| 5 | 13594097 | T/C |
| 5 | 13610001 | G/A |
| 5 | 13645752 | A/G |
| 5 | 13655428 | G/C |
| 5 | 13656983 | G/C |
| 5 | 13680807 | A/T |
| 5 | 13706619 | G/A |
| 5 | 13717618 | G/A |
| 5 | 13743168 | G/T |
| 5 | 13772064 | C/T |
| 5 | 13807115 | C/T |
| 5 | 13831461 | G/A |
| 5 | 13849577 | T/G |
| 5 | 13881832 | T/C |
| 5 | 13886551 | G/A |
| 5 | 13894570 | G/A |
| 5 | 13921461 | G/A |
| 5 | 13931435 | G/A |
| 5 | 13964506 | C/T |
| 5 | 14016414 | G/A |
| 5 | 14020860 | A/C |
| 5 | 14048035 | C/T |
| 5 | 14062809 | G/A |
| 5 | 14081325 | T/C |
| 5 | 14108884 | A/C |
| 5 | 14165112 | T/C |
| 5 | 14177625 | G/C |
| 5 | 14205724 | G/T |
| 5 | 14209716 | A/T |
| 5 | 14249056 | T/A |
| 5 | 14267833 | A/C |
| 5 | 14304874 | C/T |
| 5 | 14318401 | T/C |
| 5 | 14338876 | C/T |
| 5 | 14354523 | C/T |
| 5 | 14372445 | C/T |
| 5 | 14411295 | T/C |
| 5 | 14436396 | C/A |
| 5 | 14464473 | G/A |
| 5 | 14481788 | C/T |
| 5 | 14515064 | C/T |
| 5 | 14536966 | T/C |
| 5 | 14542623 | A/T |
| 5 | 14568143 | G/A |
| 5 | 14592191 | G/A |
| 5 | 14616878 | C/T |
| 5 | 14627777 | A/G |
| 5 | 14658996 | T/C |
| 5 | 14667377 | T/C |
| 5 | 14674814 | C/T |
| 5 | 14688404 | C/G |
| 5 | 14716544 | G/C |
| 5 | 14747902 | C/G |
| 5 | 14783484 | T/G |
| 5 | 14793274 | G/C |
| 5 | 14847143 | T/C |
| 5 | 14853024 | G/C |
| 5 | 14875243 | A/C |
| 5 | 14881024 | A/G |
| 5 | 14886998 | T/A |
| 5 | 14920476 | T/C |
| 5 | 14928375 | A/T |
| 5 | 14946020 | T/G |
| 5 | 14956675 | T/C |
| 5 | 14970919 | C/T |
| 5 | 14982909 | A/T |
| 5 | 15016282 | T/A |
| 5 | 15024951 | C/A |
| 5 | 15030393 | T/A |
| 5 | 15051945 | T/C |
| 5 | 15081591 | T/C |
| 5 | 15086362 | G/C |
| 5 | 15093674 | G/A |
| 5 | 15127066 | A/T |
| 5 | 15164770 | G/A |
| 5 | 15175118 | C/A |
| 5 | 15192278 | A/G |
| 5 | 15194473 | C/T |
| 5 | 15210892 | C/T |
| 5 | 15265328 | G/A |
| 5 | 15266600 | G/A |
| 5 | 15280599 | G/C |
| 5 | 15290477 | G/A |
| 5 | 15316092 | G/C |
| 5 | 15322380 | A/C |
| 5 | 15330182 | A/G |
| 5 | 15340296 | T/C |
| 5 | 15364521 | C/T |
| 5 | 15379360 | T/C |
| 5 | 15399045 | G/A |
| 5 | 15423440 | T/G |
| 5 | 15424503 | C/G |
| 5 | 15509530 | T/A |
| 5 | 15525684 | T/C |
| 5 | 15558199 | C/A |
| 5 | 15562190 | A/G |
| 5 | 15580367 | T/C |
| 5 | 15605910 | C/T |
| 5 | 15641389 | C/A |
| 5 | 15719046 | G/C |
| 5 | 15720047 | G/T |
| 5 | 15723514 | G/C |
| 5 | 15770651 | G/C |
| 5 | 15775991 | T/C |
| 5 | 15783203 | A/C |
| 5 | 15831488 | C/T |
| 5 | 15834857 | G/T |
| 5 | 15838677 | A/G |
| 5 | 15862186 | G/C |
| 5 | 15881168 | G/A |
| 5 | 15901512 | G/A |
| 5 | 15911010 | G/A |
| 5 | 15932298 | A/G |
| 5 | 15939511 | A/G |
| 5 | 15960339 | A/G |
| 5 | 16003641 | T/C |
| 5 | 16020299 | A/G |
| 5 | 16054349 | G/A |
| 5 | 16064987 | T/C |
| 5 | 16104641 | C/A |
| 5 | 16111364 | G/A |
| 5 | 16114236 | A/C |
| 5 | 16147620 | G/A |
| 5 | 16159856 | T/G |
| 5 | 16183114 | G/A |
| 5 | 16208690 | G/A |
| 5 | 16214939 | A/G |
| 5 | 16239126 | C/T |
| 5 | 16242049 | G/A |
| 5 | 16280298 | A/C |
| 5 | 16290413 | T/C |
| 5 | 16326981 | G/A |
| 5 | 16345071 | C/T |
| 5 | 16364825 | A/G |
| 5 | 16380922 | A/T |
| 5 | 16382503 | C/T |
| 5 | 16410370 | T/G |
| 5 | 16422198 | T/A |
| 5 | 16440784 | T/G |
| 5 | 16480655 | C/A |
| 5 | 16491266 | C/T |
| 5 | 16507358 | C/T |
| 5 | 16537056 | C/T |
| 5 | 16542778 | T/G |
| 5 | 16571927 | A/G |
| 5 | 16608416 | A/G |
| 5 | 16610431 | A/G |
| 5 | 16620859 | G/T |
| 5 | 16627006 | C/T |
| 5 | 16662224 | G/A |
| 5 | 16715122 | G/T |
| 5 | 16723551 | G/A |
| 5 | 16740089 | G/A |
| 5 | 16751801 | G/C |
| 5 | 16772634 | G/A |
| 5 | 16793312 | A/G |
| 5 | 16800066 | C/G |
| 5 | 16830499 | C/T |
| 5 | 16870548 | T/A |
| 5 | 16918197 | A/G |
| 5 | 16919391 | A/C |
| 5 | 16922902 | G/A |
| 5 | 16960555 | A/G |
| 5 | 16976495 | C/T |
| 5 | 16987036 | G/A |
| 5 | 17002578 | T/C |
| 5 | 17010288 | A/C |
| 5 | 17045345 | A/G |
| 5 | 17048366 | C/T |
| 5 | 17081666 | T/C |
| 5 | 17120329 | T/C |
| 5 | 17144503 | C/G |
| 5 | 17182987 | G/A |
| 5 | 17185947 | G/C |
| 5 | 17211668 | G/A |
| 5 | 17217177 | C/G |
| 5 | 17224666 | A/G |
| 5 | 17259610 | G/A |
| 5 | 17268331 | C/G |
| 5 | 17298092 | G/A |
| 5 | 17312091 | C/G |
| 5 | 17369799 | T/C |
| 5 | 17393241 | T/G |
| 5 | 17395876 | G/T |
| 5 | 17400959 | G/A |
| 5 | 17415879 | A/G |
| 5 | 17439887 | T/G |
| 5 | 17451541 | G/A |
| 5 | 17464829 | C/T |
| 5 | 17477493 | G/A |
| 5 | 17504365 | C/G |
| 5 | 17521010 | G/A |
| 5 | 17535714 | G/T |
| 5 | 17551076 | G/C |
| 5 | 17593166 | C/T |
| 5 | 17606394 | T/C |
| 5 | 17612957 | G/A |
| 5 | 17633634 | A/G |
| 5 | 17640083 | C/A |
| 5 | 17649326 | C/T |
| 5 | 17670813 | T/G |
| 5 | 17690000 | G/A |
| 5 | 17712750 | T/C |
| 5 | 17748786 | G/A |
| 5 | 17751276 | A/G |
| 5 | 17779637 | G/A |
| 5 | 17780706 | T/C |
| 5 | 17791201 | G/A |
| 5 | 17806720 | C/T |
| 5 | 17844038 | C/A |
| 5 | 17858063 | A/G |
| 5 | 17881456 | G/C |
| 5 | 17902731 | G/C |
| 5 | 17927542 | T/C |
| 5 | 17934292 | C/G |
| 5 | 17965815 | T/G |
| 5 | 17972085 | A/G |
| 5 | 17987694 | G/A |
| 5 | 18019927 | A/G |
| 5 | 18032299 | T/C |
| 5 | 18055317 | G/C |
| 5 | 18060883 | A/T |
| 5 | 18091175 | A/C |
| 5 | 18130066 | C/A |
| 5 | 18154487 | C/G |
| 5 | 18167954 | A/G |
| 5 | 18202241 | A/G |
| 5 | 18230545 | T/C |
| 5 | 18268116 | C/T |
| 5 | 18270867 | T/A |
| 5 | 18282238 | C/T |
| 5 | 18305865 | T/C |
| 5 | 18310154 | A/G |
| 5 | 18341763 | C/T |
| 5 | 18353639 | T/C |
| 5 | 18378996 | G/A |
| 5 | 18396918 | T/G |
| 5 | 18422050 | A/G |
| 5 | 18426062 | C/G |
| 5 | 18451473 | G/A |
| 5 | 18460402 | G/A |
| 5 | 18489831 | C/A |
| 5 | 18500303 | G/T |
| 5 | 18525710 | A/C |
| 5 | 18537690 | C/G |
| 5 | 18544230 | G/A |
| 5 | 18557387 | T/C |
| 5 | 18584952 | A/T |
| 5 | 18600199 | A/G |
| 5 | 18604474 | C/G |
| 5 | 18632259 | C/T |
| 5 | 18654181 | C/T |
| 5 | 18669266 | G/A |
| 5 | 18686263 | T/C |
| 5 | 18690076 | T/C |
| 5 | 18704395 | T/C |
| 5 | 18726104 | C/T |
| 5 | 18731869 | G/A |
| 5 | 18758915 | C/T |
| 5 | 18775627 | C/T |
| 5 | 18784772 | A/G |
| 5 | 18789067 | G/A |
| 5 | 18810698 | A/G |
| 5 | 18863567 | G/A |
| 5 | 18894402 | G/A |
| 5 | 18992252 | C/T |
| 5 | 18995897 | C/T |
| 5 | 19065925 | C/T |
| 5 | 19092066 | C/T |
| 5 | 19095383 | T/A |
| 5 | 19122715 | C/T |
| 5 | 19127332 | A/T |
| 5 | 19160603 | T/C |
| 5 | 19175577 | C/T |
| 5 | 19188011 | C/G |
| 5 | 19226617 | C/T |
| 5 | 19232682 | A/C |
| 5 | 19236216 | T/C |
| 5 | 19286975 | T/A |
| 5 | 19295415 | A/G |
| 5 | 19308992 | A/G |
| 5 | 19321927 | C/T |
| 5 | 19403670 | G/A |
| 5 | 19418544 | G/A |
| 5 | 19441453 | C/G |
| 5 | 19460832 | C/A |
| 5 | 19489542 | C/T |
| 5 | 19517424 | A/G |
| 5 | 19546943 | T/A |
| 5 | 19586120 | G/T |
| 5 | 19600504 | G/A |
| 5 | 19601951 | C/T |
| 5 | 19625875 | C/T |
| 5 | 19637690 | C/G |
| 5 | 19673915 | T/G |
| 5 | 19676784 | G/A |
| 5 | 19692627 | A/C |
| 5 | 19734905 | C/T |
| 5 | 19753047 | A/G |
| 5 | 19757502 | T/C |
| 5 | 19771563 | A/G |
| 5 | 19805173 | C/G |
| 5 | 19831469 | C/T |
| 5 | 19833818 | G/A |
| 5 | 19872833 | A/C |
| 5 | 19906330 | T/C |
| 5 | 19923017 | T/C |
| 5 | 19944843 | T/C |
| 5 | 19961620 | T/C |
| 5 | 19993158 | A/C |
| 5 | 19996405 | T/G |
| 5 | 20035017 | A/G |
| 5 | 20061991 | A/G |
| 5 | 20065483 | T/C |
| 5 | 20076668 | C/A |
| 5 | 20097446 | T/C |
| 5 | 20102099 | C/T |
| 5 | 20109496 | T/C |
| 5 | 20146385 | T/G |
| 5 | 20153052 | T/C |
| 5 | 20175444 | A/G |
| 5 | 20182395 | T/G |
| 5 | 20215034 | C/A |
| 5 | 20218441 | G/T |
| 5 | 20238583 | G/A |
| 5 | 20245574 | A/G |
| 5 | 20279942 | C/T |
| 5 | 20331558 | G/A |
| 5 | 20347945 | C/T |
| 5 | 20349291 | T/G |
| 5 | 20392548 | C/A |
| 5 | 20417641 | A/C |
| 5 | 20438623 | G/T |
| 5 | 20450630 | G/A |
| 5 | 20469216 | A/C |
| 5 | 20484801 | G/A |
| 5 | 20508469 | A/G |
| 5 | 20519035 | G/A |
| 5 | 20531734 | A/G |
| 5 | 20561222 | C/A |
| 5 | 20582852 | A/C |
| 5 | 20603537 | T/C |
| 5 | 20625885 | C/T |
| 5 | 20646053 | G/A |
| 5 | 20649245 | C/T |
| 5 | 20672239 | C/T |
| 5 | 20691257 | A/T |
| 5 | 20701919 | C/T |
| 5 | 20728457 | T/A |
| 5 | 20755634 | C/T |
| 5 | 20771640 | A/G |
| 5 | 20799674 | T/C |
| 5 | 20806294 | C/T |
| 5 | 20820672 | T/C |
| 5 | 20855451 | C/T |
| 5 | 20872921 | G/C |
| 5 | 20890319 | A/G |
| 5 | 20900932 | T/C |
| 5 | 20913019 | G/C |
| 5 | 20939622 | A/G |
| 5 | 20959943 | A/G |
| 5 | 20960947 | C/T |
| 5 | 20985506 | T/G |
| 5 | 20992392 | A/C |
| 5 | 21013992 | G/A |
| 5 | 21031371 | G/A |
| 5 | 21049503 | C/T |
| 5 | 21062758 | T/C |
| 5 | 21080868 | G/A |
| 5 | 21098940 | C/T |
| 5 | 21101693 | C/T |
| 5 | 21127382 | G/T |
| 5 | 21147538 | G/A |
| 5 | 21156393 | A/C |
| 5 | 21162974 | G/A |
| 5 | 21181386 | G/A |
| 5 | 21201266 | G/A |
| 5 | 21218137 | T/G |
| 5 | 21225273 | C/A |
| 5 | 21278999 | T/C |
| 5 | 21328067 | T/C |
| 5 | 21345930 | C/T |
| 5 | 21350046 | C/T |
| 5 | 21386561 | C/T |
| 5 | 21395071 | C/T |
| 5 | 21447041 | C/T |
| 5 | 21438872 | G/A |
| 5 | 21463082 | G/A |
| 5 | 21480891 | C/T |
| 5 | 21493120 | G/A |
| 5 | 31517642 | C/T |
| 5 | 21539149 | C/T |
| 5 | 21540303 | C/T |
| 5 | 21553054 | T/G |
| 5 | 21594320 | C/A |
| 5 | 21603246 | T/C |
| 5 | 21633555 | C/T |
| 5 | 21659625 | C/T |
| 5 | 21682721 | C/T |
| 5 | 21716140 | C/T |
| 5 | 21734960 | C/T |
| 5 | 21738702 | G/A |
| 5 | 21755394 | C/T |
| 5 | 21759078 | C/T |
| 5 | 21809810 | T/C |
| 5 | 21827020 | C/T |
| 5 | 21895786 | C/G |
| 5 | 21918249 | G/A |
| 5 | 21926365 | C/T |
| 5 | 21934630 | G/A |
| 5 | 21969487 | C/T |
| 5 | 21975138 | C/T |
| 5 | 21994722 | T/G |
| 5 | 22047625 | C/T |
| 5 | 22058097 | T/C |
| 5 | 22079531 | C/A |
| 5 | 22132380 | A/T |
| 5 | 22147867 | G/A |
| 5 | 22159446 | C/G |
| 5 | 22222100 | C/T |
| 5 | 22249549 | C/T |
| 5 | 22253045 | G/A |
| 5 | 22272783 | T/G |
| 5 | 22339789 | G/A |
| 5 | 22350049 | C/G |
| 5 | 22402062 | T/C |
| 5 | 22439250 | C/T |
| 5 | 22473522 | C/T |
| 5 | 22480626 | C/T |
| 5 | 22509555 | T/A |
| 5 | 22519134 | G/T |
| 5 | 22536413 | C/G |
| 5 | 22571952 | T/C |
| 5 | 22580080 | C/T |
| 5 | 22609322 | G/A |
| 5 | 22611535 | C/T |
| 5 | 22627529 | C/T |
| 5 | 22688998 | G/A |
| 5 | 22728801 | C/T |
| 5 | 22752893 | G/A |
| 5 | 22756220 | T/C |
| 5 | 22783553 | T/C |
| 5 | 22823408 | A/T |
| 5 | 22847414 | C/T |
| 5 | 22885036 | A/C |
| 5 | 22899571 | A/G |
| 5 | 22922429 | C/A |
| 5 | 22971311 | A/G |
| 5 | 22987652 | G/A |
| 5 | 23051580 | G/A |
| 5 | 23153041 | A/G |
| 5 | 23170392 | G/A |
| 5 | 23358111 | G/A |
| 5 | 23426928 | G/A |
| 5 | 23444353 | G/A |
| 5 | 23465317 | C/T |
| 5 | 23491387 | T/A |
| 5 | 23535318 | T/C |
| 5 | 23575607 | C/T |
| 5 | 23601434 | C/A |
| 5 | 23664645 | C/T |
| 5 | 23698015 | A/G |
| 5 | 23746458 | G/A |
| 5 | 23748659 | C/T |
| 5 | 23788139 | C/T |
| 5 | 23793365 | G/C |
| 5 | 23843355 | G/A |
| 5 | 23912433 | A/T |
| 5 | 23946370 | C/T |
| 5 | 23956657 | C/T |
| 5 | 23986019 | C/T |
| 5 | 24015123 | C/T |
| 5 | 24039964 | C/T |
| 5 | 24071307 | C/G |
| 5 | 24078725 | C/T |
| 5 | 24091817 | C/T |
| 5 | 24194891 | G/A |
| 5 | 24241136 | A/G |
| 5 | 24308863 | A/G |
| 5 | 24330461 | G/A |
| 5 | 24334875 | C/A |
| 5 | 24369490 | G/T |
| 5 | 24402144 | A/C |
| 5 | 24407673 | T/G |
| 5 | 24437111 | A/G |
| 5 | 24468751 | G/A |
| 5 | 24507183 | G/T |
| 5 | 24529682 | A/T |
| 5 | 24588103 | A/G |
| 5 | 24604921 | G/A |
| 5 | 24648596 | C/A |
| 5 | 24692492 | T/C |
| 5 | 24703283 | G/T |
| 5 | 24758074 | G/C |
| 5 | 24768886 | C/G |
| 5 | 24792560 | G/A |
| 5 | 24795997 | G/A |
| 5 | 24867208 | C/T |
| 5 | 24888886 | C/G |
| 5 | 24923530 | A/G |
| 5 | 24943378 | A/C |
| 5 | 25014967 | A/G |
| 5 | 25041902 | C/T |
| 5 | 25051708 | C/T |
| 5 | 25053771 | C/T |
| 5 | 25092162 | G/T |
| 5 | 25127776 | G/A |
| 5 | 25184467 | G/A |
| 5 | 25194253 | C/T |
| 5 | 25221065 | A/G |
| 5 | 25251726 | C/T |
| 5 | 25256344 | C/T |
| 5 | 25290462 | G/C |
| 5 | 25391164 | C/T |
| 5 | 25394171 | G/A |
| 5 | 25453308 | C/T |
| 5 | 25497609 | C/T |
| 5 | 25516009 | C/T |
| 5 | 25537220 | C/T |
| 5 | 25542743 | T/C |
| 5 | 25580140 | G/A |
| 5 | 25597754 | G/A |
| 5 | 25602983 | A/G |
| 5 | 25667617 | T/A |
| 5 | 25682914 | A/T |
| 5 | 25730569 | G/A |
| 5 | 25766322 | C/T |
| 5 | 25782297 | T/G |
| 5 | 25784590 | C/T |
| 5 | 25805612 | G/A |
| 5 | 25854460 | C/T |
| 5 | 25857846 | T/C |
| 5 | 25868298 | C/T |
| 5 | 25904628 | G/A |
| 5 | 25919381 | A/G |
| 5 | 25933734 | G/A |
| 5 | 25969075 | C/T |
| 5 | 25978950 | A/G |
| 5 | 25992100 | C/T |
| 5 | 26041752 | G/C |
| 5 | 26076026 | T/G |
| 5 | 26133890 | G/A |
| 5 | 26168706 | A/G |
| 5 | 26216537 | C/T |
| 5 | 26229262 | C/T |
| 5 | 26249206 | G/A |
| 5 | 26282176 | C/T |
| 5 | 26315152 | C/T |
| 5 | 26322620 | C/T |
| 5 | 26355585 | C/T |
| 5 | 26373816 | C/T |
| 5 | 26385059 | A/G |
| 5 | 26426489 | G/A |
| 5 | 26432848 | A/C |
| 5 | 26438512 | G/T |
| 5 | 26484951 | G/A |
| 5 | 26487336 | T/A |
| 5 | 26494873 | C/T |
| 5 | 26512524 | G/A |
| 5 | 26525688 | G/A |
| 5 | 26582476 | G/A |
| 5 | 26583543 | T/A |
| 5 | 26610120 | A/G |
| 5 | 26662899 | T/A |
| 5 | 26682067 | C/T |
| 5 | 26759300 | G/A |
| 5 | 26800952 | A/T |
| 5 | 26840448 | C/T |
| 5 | 26850998 | T/C |
| 5 | 26901890 | G/C |
| 5 | 26922686 | T/C |
| 5 | 26949677 | C/T |
| 5 | 26951292 | C/G |
| 5 | 26979935 | T/A |
| 5 | 27027239 | C/T |
| 5 | 27052527 | T/C |
| 5 | 27055628 | C/T |
| 5 | 27062691 | C/T |
| 5 | 27073770 | G/A |
| 5 | 27107701 | G/A |
| 5 | 27141606 | A/G |
| 5 | 27146333 | A/G |
| 5 | 27174888 | A/G |
| 5 | 27186829 | G/A |
| 5 | 27192797 | C/T |
| 5 | 27220861 | T/C |
| 5 | 27233747 | G/A |
| 5 | 27242752 | T/C |
| 5 | 27271133 | C/T |
| 5 | 27284175 | C/T |
| 5 | 27301212 | C/G |
| 5 | 27302874 | C/T |
| 5 | 27335307 | G/T |
| 5 | 27359716 | T/C |
| 5 | 27368493 | T/C |
| 5 | 27407962 | A/C |
| 5 | 27423519 | A/G |
| 5 | 27457578 | T/C |
| 5 | 27503205 | T/G |
| 5 | 27521085 | T/C |
| 5 | 27533261 | T/C |
| 5 | 27569605 | A/C |
| 5 | 27575021 | G/A |
| 5 | 27592070 | T/C |
| 5 | 27607102 | T/C |
| 5 | 27636442 | C/T |
| 5 | 27649429 | C/A |
| 5 | 27669140 | G/A |
| 5 | 27681335 | C/T |
| 5 | 27708695 | T/G |
| 5 | 27726022 | T/A |
| 5 | 27739711 | T/C |
| 5 | 27765920 | C/T |
| 5 | 27796728 | G/A |
| 5 | 27804787 | C/T |
| 5 | 27818510 | T/C |
| 5 | 27828669 | C/T |
| 5 | 27855708 | T/C |
| 5 | 27864773 | T/C |
| 5 | 27878435 | C/T |
| 5 | 27919636 | G/A |
| 5 | 27926072 | A/G |
| 5 | 27950491 | C/T |
| 5 | 27961994 | C/T |
| 5 | 27965136 | G/A |
| 5 | 28010766 | G/A |
| 5 | 28026479 | T/C |
| 5 | 28047276 | C/T |
| 5 | 28062537 | C/T |
| 5 | 28065802 | C/G |
| 5 | 28085494 | T/G |
| 5 | 28098873 | A/T |
| 5 | 28114043 | G/A |
| 5 | 28121270 | C/T |
| 5 | 28153916 | C/T |
| 5 | 28155173 | C/A |
| 5 | 28182635 | A/G |
| 5 | 28193863 | C/T |
| 5 | 28290614 | G/C |
| 5 | 28326068 | C/A |
| 5 | 28407647 | G/A |
| 5 | 28417753 | A/G |
| 5 | 28438702 | G/A |
| 5 | 28468097 | C/T |
| 5 | 28487682 | G/A |
| 5 | 28490144 | C/T |
| 5 | 28516917 | G/A |
| 5 | 28534738 | G/T |
| 5 | 28540569 | T/G |
| 5 | 28584461 | C/T |
| 5 | 28598718 | G/A |
| 5 | 28602320 | C/T |
| 5 | 28664051 | A/G |
| 5 | 28705725 | G/A |
| 5 | 28742359 | C/T |
| 5 | 28776751 | T/A |
| 5 | 28787836 | G/A |
| 5 | 28787837 | G/A |
| 5 | 28787838 | G/A |
| 5 | 28787839 | G/A |
| 5 | 28841750 | C/T |
| 5 | 28872535 | T/C |
| 5 | 28891437 | C/T |
| 5 | 28930253 | C/A |
| 5 | 28970182 | T/C |
| 5 | 28974420 | C/T |
| 5 | 29001813 | C/A |
| 5 | 29003378 | A/G |
| 5 | 29015637 | A/G |
| 5 | 29017359 | G/A |
| 5 | 29047045 | C/T |
| 5 | 29073929 | G/A |
| 5 | 29146768 | C/T |
| 5 | 29177372 | A/C |
| 5 | 29178527 | A/C |
| 5 | 29219593 | C/A |
| 5 | 29223733 | A/T |
| 5 | 29227194 | T/C |
| 5 | 29277381 | A/T |
| 5 | 29360072 | C/T |
| 5 | 29397051 | C/T |
| 5 | 29399574 | G/A |
| 5 | 29413315 | C/A |
| 5 | 29444992 | G/A |
| 5 | 29455152 | C/A |
| 5 | 29474210 | C/T |
| 5 | 29478227 | G/A |
| 5 | 29499241 | C/T |
| 5 | 29504260 | C/T |
| 5 | 29546258 | G/A |
| 5 | 29569706 | C/T |
| 5 | 29573214 | T/G |
| 5 | 29583524 | G/C |
| 5 | 29628008 | G/A |
| 5 | 29639376 | C/G |
| 5 | 29645420 | C/T |
| 5 | 29659459 | A/G |
| 5 | 29675359 | C/T |
| 5 | 29720964 | T/C |
| 5 | 29755974 | A/G |
| 5 | 29765525 | G/T |
| 5 | 29792528 | G/A |
| 5 | 29838680 | T/C |
| 5 | 29861976 | T/C |
| 5 | 29864873 | A/G |
| 5 | 29909909 | A/G |
| 5 | 29920388 | G/A |
| 5 | 29963386 | A/T |
| 5 | 29968518 | A/T |
| 5 | 29971892 | A/G |
| 5 | 29982842 | A/G |
| 5 | 30005472 | G/A |
| 5 | 30015704 | C/T |
| 5 | 30030098 | G/C |
| 5 | 30038375 | G/A |
| 5 | 30100391 | A/G |
| 5 | 30103988 | C/G |
| 5 | 30132002 | C/A |
| 5 | 30152743 | C/G |
| 5 | 30187995 | G/A |
| 5 | 30238399 | G/A |
| 5 | 30244155 | T/C |
| 5 | 30246212 | C/T |
| 5 | 30272427 | T/C |
| 5 | 30283406 | A/G |
| 5 | 30318008 | T/C |
| 5 | 30323413 | A/G |
| 5 | 30348249 | T/G |
| 5 | 30375988 | A/G |
| 5 | 30388466 | A/G |
| 5 | 30396490 | C/G |
| 5 | 30423824 | G/A |
| 5 | 30426552 | G/C |
| 5 | 30459628 | T/C |
| 5 | 30462562 | G/A |
| 5 | 30496578 | T/G |
| 5 | 30505202 | A/G |
| 5 | 30522193 | T/C |
| 5 | 30550829 | T/C |
| 5 | 30570637 | T/C |
| 5 | 30599339 | G/A |
| 5 | 30602549 | A/C |
| 5 | 30607268 | T/C |
| 5 | 30647507 | G/A |
| 5 | 30679094 | G/C |
| 5 | 20682188 | T/C |
| 5 | 30697152 | G/A |
| 5 | 30722857 | G/A |
| 5 | 30743525 | T/G |
| 5 | 30773088 | A/G |
| 5 | 30776024 | T/G |
| 5 | 30785119 | T/C |
| 5 | 30795671 | C/T |
| 5 | 30816143 | A/C |
| 5 | 30824431 | C/T |
| 5 | 30848444 | T/A |
| 5 | 30849803 | T/C |
| 5 | 30888617 | C/T |
| 5 | 30889974 | G/A |
| 5 | 30922070 | G/A |
| 5 | 30938863 | T/C |
| 5 | 30965427 | T/G |
| 5 | 30995055 | A/T |
| 5 | 31009024 | T/A |
| 5 | 31020064 | C/T |
| 5 | 31045497 | T/C |
| 5 | 31051098 | G/A |
| 5 | 31052827 | T/C |
| 5 | 31082625 | G/A |
| 5 | 31097054 | A/C |
| 5 | 31119916 | G/A |
| 5 | 31158239 | T/C |
| 5 | 31181317 | A/C |
| 5 | 31195313 | A/G |
| 5 | 31208294 | G/A |
| 5 | 31233309 | G/A |
| 5 | 31259768 | C/T |
| 5 | 31278081 | C/T |
| 5 | 31300816 | T/G |
| 5 | 31304804 | G/A |
| 5 | 31324748 | T/C |
| 5 | 31327136 | G/A |
| 5 | 31362444 | T/C |
| 5 | 31389693 | C/T |
| 5 | 31417300 | A/T |
| 5 | 31437171 | C/T |
| 5 | 31460645 | G/A |
| 5 | 31498698 | T/C |
| 5 | 31501529 | A/G |
| 5 | 31533569 | A/G |
| 5 | 31583276 | C/A |
| 5 | 31598880 | G/A |
| 5 | 31612886 | G/C |
| 5 | 31627366 | A/T |
| 5 | 31645372 | G/T |
| 5 | 31650315 | A/C |
| 5 | 31677409 | T/C |
| 5 | 31677410 | C/G |
| 5 | 31677411 | C/G |
| 5 | 31677412 | C/G |
| 5 | 31720240 | G/A |
| 5 | 31724970 | C/T |
| 5 | 31724971 | C/T |
| 5 | 31724972 | C/T |
| 5 | 31783658 | C/T |
| 5 | 31792594 | G/A |
| 5 | 31800284 | C/T |
| 5 | 31839752 | G/A |
| 5 | 31947090 | C/T |
| 5 | 31949299 | C/G |
| 5 | 31967041 | C/G |
| 5 | 31990243 | C/T |
| 5 | 32021229 | G/A |
| 5 | 32024215 | A/G |
| 5 | 32043496 | G/T |
| 5 | 32045336 | G/A |
| 5 | 32095664 | A/G |
| 5 | 32102994 | G/A |
| 5 | 32146005 | G/A |
| 5 | 32154926 | T/C |
| 5 | 32184306 | C/A |
| 5 | 32194527 | T/C |
| 5 | 32227563 | G/A |
| 5 | 32250204 | G/C |
| 5 | 32262546 | G/T |
| 5 | 32284468 | G/A |
| 5 | 32306456 | A/G |
| 5 | 32313707 | A/T |
| 5 | 32318103 | T/C |
| 5 | 32347229 | G/A |
| 5 | 32378083 | C/T |
| 5 | 32426890 | T/C |
| 5 | 32459778 | G/T |
| 5 | 32511244 | C/T |
| 5 | 32545394 | G/T |
| 5 | 32553858 | C/A |
| 5 | 32592766 | G/T |
| 5 | 32600789 | C/T |
| 5 | 32613692 | A/G |
| 5 | 32615779 | C/T |
| 5 | 32633941 | C/T |
| 5 | 32671451 | A/G |
| 5 | 32706374 | A/G |
| 5 | 32726569 | T/G |
| 5 | 32750779 | C/T |
| 5 | 32758882 | A/G |
| 5 | 32767666 | T/C |
| 5 | 32790926 | T/C |
| 5 | 32832976 | G/T |
| 5 | 32850669 | T/C |
| 5 | 32885787 | A/G |
| 5 | 32903897 | G/A |
| 5 | 32915938 | C/T |
| 5 | 32933434 | C/A |
| 5 | 32945317 | C/T |
| 5 | 32949169 | C/T |
| 5 | 32981184 | T/C |
| 5 | 33047859 | G/T |
| 5 | 33066701 | C/T |
| 5 | 33081225 | A/G |
| 5 | 33095256 | G/A |
| 5 | 33146466 | T/C |
| 5 | 33177086 | C/A |
| 5 | 33196664 | T/C |
| 5 | 33223511 | G/A |
| 5 | 33247302 | G/C |
| 5 | 33252716 | C/T |
| 5 | 33280828 | G/A |
| 5 | 33295724 | C/T |
| 5 | 33316982 | G/T |
| 5 | 33323740 | G/A |
| 5 | 33336599 | G/T |
| 5 | 33379044 | T/G |
| 5 | 33396257 | T/G |
| 5 | 33400138 | G/A |
| 5 | 33422977 | T/C |
| 5 | 33450538 | T/A |
| 5 | 33484650 | A/C |
| 5 | 33515344 | G/C |
| 5 | 33517224 | G/C |
| 5 | 33551295 | A/G |
| 5 | 33553588 | A/G |
| 5 | 33591926 | C/T |
| 5 | 33598843 | G/A |
| 5 | 33602962 | A/G |
| 5 | 33627820 | A/G |
| 5 | 33683059 | A/G |
| 5 | 33691712 | G/A |
| 5 | 33723337 | C/G |
| 5 | 33733670 | C/T |
| 5 | 33751974 | G/A |
| 5 | 33774911 | G/T |
| 5 | 33786894 | A/T |
| 5 | 33814340 | G/T |
| 5 | 33823996 | C/T |
| 5 | 33866043 | C/A |
| 5 | 33886181 | C/T |
| 5 | 33888217 | C/T |
| 5 | 33907069 | T/G |
| 5 | 33924090 | T/G |
| 5 | 33996625 | T/C |
| 5 | 34040944 | G/A |
| 5 | 34071998 | A/T |
| 5 | 34099185 | G/A |
| 5 | 34099891 | T/G |
| 5 | 34100969 | T/A |
| 5 | 34112725 | T/C |
| 5 | 34116591 | G/A |
| 5 | 34167668 | A/G |
| 5 | 34172449 | A/G |
| 5 | 34180222 | T/C |
| 5 | 34223961 | T/C |
| 5 | 34256472 | G/C |
| 5 | 34267068 | G/A |
| 5 | 34293484 | G/A |
| 5 | 34310827 | G/T |
| 5 | 34340600 | C/T |
| 5 | 34359293 | C/T |
| 5 | 34391548 | C/G |
| 5 | 34421623 | G/A |
| 5 | 34450712 | G/A |
| 5 | 34489187 | G/A |
| 5 | 34511940 | A/T |
| 5 | 34518479 | T/A |
| 5 | 34541120 | A/G |
| 5 | 34589014 | C/T |
| 5 | 34635795 | C/T |
| 5 | 34701996 | C/T |
| 5 | 34713993 | G/A |
| 5 | 34736101 | A/G |
| 5 | 34741250 | C/T |
| 5 | 34768229 | G/C |
| 5 | 34789922 | C/T |
| 5 | 34821969 | G/A |
| 5 | 34839794 | C/T |
| 5 | 34846215 | C/T |
| 5 | 34879030 | G/A |
| 5 | 34894208 | G/A |
| 5 | 34897826 | G/A |
| 5 | 34921221 | C/G |
| 5 | 34997634 | G/A |
| 5 | 35006358 | C/T |
| 5 | 35022568 | G/A |
| 5 | 35031504 | G/T |
| 5 | 35050455 | G/A |
| 5 | 35052958 | T/C |
| 5 | 35073096 | G/A |
| 5 | 35088271 | T/C |
| 5 | 35127078 | G/A |
| 5 | 35180732 | C/T |
| 5 | 35210763 | C/T |
| 5 | 35227998 | A/G |
| 5 | 35256249 | C/G |
| 5 | 35276559 | A/T |
| 5 | 35300223 | G/A |
| 5 | 35332437 | C/T |
| 5 | 35341969 | G/A |
| 5 | 35416536 | C/T |
| 5 | 35482618 | G/A |
| 5 | 35490651 | G/T |
| 5 | 35510744 | A/T |
| 5 | 35532321 | T/C |
| 5 | 35539523 | C/T |
| 5 | 35562722 | A/G |
| 5 | 35578299 | G/A |
| 5 | 35582101 | A/G |
| 5 | 35630038 | T/C |
| 5 | 35631378 | A/T |
| 5 | 35646265 | T/A |
| 5 | 35652377 | T/A |
| 5 | 35678390 | C/G |
| 5 | 35700728 | A/C |
| 5 | 35705619 | G/A |
| 5 | 35732227 | C/T |
| 5 | 35763451 | A/C |
| 5 | 35806213 | T/C |
| 5 | 35821918 | G/A |
| 5 | 35853733 | G/A |
| 5 | 35916262 | G/A |
| 5 | 35924824 | A/T |
| 5 | 35965916 | A/G |
| 5 | 35994553 | C/T |
| 5 | 36002853 | A/C |
| 5 | 36048025 | T/A |
| 5 | 36061202 | C/T |
| 5 | 36123547 | G/A |
| 5 | 36162129 | G/A |
| 5 | 36170514 | G/A |
| 5 | 36189661 | T/C |
| 5 | 36235449 | C/T |
| 5 | 36274289 | C/A |
| 5 | 36361717 | G/A |
| 5 | 36401021 | G/A |
| 5 | 36446442 | G/A |
| 5 | 36449877 | T/A |
| 5 | 36451001 | T/C |
| 5 | 36458184 | A/T |
| 5 | 36491759 | G/A |
| 5 | 36515544 | A/G |
| 5 | 36537046 | T/C |
| 5 | 36550978 | A/G |
| 5 | 36573676 | C/T |
| 5 | 36595316 | C/G |
| 5 | 36624986 | A/G |
| 5 | 36637741 | C/T |
| 5 | 36652937 | C/A |
| 5 | 36669855 | C/G |
| 5 | 36671847 | C/A |
| 5 | 36702052 | G/C |
| 5 | 36705960 | G/A |
| 5 | 36729076 | C/T |
| 5 | 36731470 | T/G |
| 5 | 36763496 | C/A |
| 5 | 36772688 | G/A |
| 5 | 36780917 | A/C |
| 5 | 36816930 | T/G |
| 5 | 36825418 | C/T |
| 5 | 36840323 | G/C |
| 5 | 36872204 | G/C |
| 5 | 36875646 | C/T |
| 5 | 36904194 | G/A |
| 5 | 36959957 | C/T |
| 5 | 36962100 | C/G |
| 5 | 37008915 | G/A |
| 5 | 37027522 | T/C |
| 5 | 37029968 | C/T |
| 5 | 37051319 | T/C |
| 5 | 37052334 | T/G |
| 5 | 37089128 | T/C |
| 5 | 37092558 | G/T |
| 5 | 37124650 | A/T |
| 5 | 37140154 | C/T |
| 5 | 37171864 | T/C |
| 5 | 37200086 | A/G |
| 5 | 37212577 | T/G |
| 5 | 37241825 | A/G |
| 5 | 37251009 | G/A |
| 5 | 37289776 | T/A |
| 5 | 37291458 | C/G |
| 5 | 37316472 | G/C |
| 5 | 37323134 | G/C |
| 5 | 37346212 | C/G |
| 5 | 37356283 | C/T |
| 5 | 37357674 | T/G |
| 5 | 37381083 | T/C |
| 5 | 37418056 | A/T |
| 5 | 37449531 | G/A |
| 5 | 37453771 | A/G |
| 5 | 37478494 | C/T |
| 5 | 37500079 | T/C |
| 5 | 37508836 | T/A |
| 5 | 37535283 | A/G |
| 5 | 37566725 | C/T |
| 5 | 37588942 | C/T |
| 5 | 37594905 | G/A |
| 5 | 37640044 | T/C |
| 5 | 37654384 | A/G |
| 5 | 37689697 | T/G |
| 5 | 37724269 | T/G |
| 5 | 37734292 | G/C |
| 5 | 37760127 | G/C |
| 5 | 37762790 | G/A |
| 5 | 37771832 | C/T |
| 5 | 37872177 | A/G |
| 5 | 37877390 | A/T |
| 5 | 37904376 | C/T |
| 5 | 37907961 | A/G |
| 5 | 37927494 | C/T |
| 5 | 37971018 | G/A |
| 5 | 37977967 | A/G |
| 5 | 37985909 | C/A |
| 5 | 38057509 | C/T |
| 5 | 38160706 | C/T |
| 5 | 38199721 | G/T |
| 5 | 38255439 | C/T |
| 5 | 38280239 | T/C |
| 5 | 38281882 | T/C |
| 5 | 38316246 | A/G |
| 5 | 38367798 | G/A |
| 5 | 38370441 | A/C |
| 5 | 38380767 | C/T |
| 5 | 38426916 | C/T |
| 5 | 38428511 | C/T |
| 5 | 38449949 | G/C |
| 5 | 38459898 | G/A |
| 5 | 38472403 | C/T |
| 5 | 38507598 | G/A |
| 5 | 38533378 | T/A |
| 5 | 38537141 | C/A |
| 5 | 38553035 | T/C |
| 5 | 38562876 | G/A |
| 5 | 38580534 | T/C |
| 5 | 38598407 | C/T |
| 5 | 38619030 | G/A |
| 5 | 38621334 | T/G |
| 5 | 38651715 | C/T |
| 5 | 38659829 | A/G |
| 5 | 38672209 | A/T |
| 5 | 38675730 | G/A |
| 5 | 38710313 | T/C |
| 5 | 38725727 | G/A |
| 5 | 38753642 | A/T |
| 5 | 38757184 | T/C |
| 5 | 38761027 | C/T |
| 5 | 38799849 | A/T |
| 5 | 38847696 | T/C |
| 5 | 38859105 | C/T |
| 5 | 38866006 | C/T |
| 5 | 38898925 | C/A |
| 5 | 38914858 | A/C |
| 5 | 38957516 | G/T |
| 5 | 38965892 | C/A |
| 5 | 38974056 | T/C |
| 5 | 38995885 | C/A |
| 5 | 39002620 | C/T |
| 5 | 39020600 | G/T |
| 5 | 39041988 | G/T |
| 5 | 39043297 | G/A |
| 5 | 39087426 | T/C |
| 5 | 39092812 | A/C |
| 5 | 39140628 | C/T |
| 5 | 39159842 | T/C |
| 5 | 39174153 | A/G |
| 5 | 39201211 | T/C |
| 5 | 39231740 | G/A |
| 5 | 39238358 | T/C |
| 5 | 39241618 | A/G |
| 5 | 39262842 | C/T |
| 5 | 39280212 | G/T |
| 5 | 39358751 | C/A |
| 5 | 39372478 | T/C |
| 5 | 39379937 | C/T |
| 5 | 39409576 | G/A |
| 5 | 39419083 | T/C |
| 5 | 39433695 | G/C |
| 5 | 39444833 | G/C |
| 5 | 39461089 | C/T |
| 5 | 39473453 | T/C |
| 5 | 39481373 | C/T |
| 5 | 39499538 | A/C |
| 5 | 39523092 | G/A |
| 5 | 39526379 | C/G |
| 5 | 39541192 | C/T |
| 5 | 36546900 | A/G |
| 5 | 39577081 | G/A |
| 5 | 39592404 | T/A |
| 5 | 39624596 | G/C |
| 5 | 39628124 | A/T |
| 5 | 39633109 | G/A |
| 5 | 39648682 | T/C |
| 5 | 39682178 | T/A |
| 5 | 39710984 | C/T |
| 5 | 39732200 | C/A |
| 5 | 39735801 | A/G |
| 5 | 39770206 | A/G |
| 5 | 39773868 | G/A |
| 5 | 39782515 | G/A |
| 5 | 39816017 | G/C |
| 5 | 39842624 | G/C |
| 5 | 39896752 | T/C |
| 5 | 39898324 | G/A |
| 5 | 39901132 | T/C |
| 5 | 39946458 | G/T |
| 5 | 39956451 | G/T |
| 5 | 39987240 | G/C |
| 5 | 40016114 | G/C |
| 5 | 40019538 | T/C |
| 5 | 40021827 | T/C |
| 5 | 40036860 | A/G |
| 5 | 40050085 | C/T |
| 5 | 40125171 | A/G |
| 5 | 40129052 | T/C |
| 5 | 40174447 | A/G |
| 5 | 40195767 | T/C |
| 5 | 40229154 | A/G |
| 5 | 40247908 | A/G |
| 5 | 40289469 | G/A |
| 5 | 40291252 | T/C |
| 5 | 40335421 | C/T |
| 5 | 40349994 | C/A |
| 5 | 40364903 | T/C |
| 5 | 40389299 | C/T |
| 5 | 40398177 | C/T |
| 5 | 40435807 | A/T |
| 5 | 40440072 | C/T |
| 5 | 40441843 | C/A |
| 5 | 40477552 | G/A |
| 5 | 40486489 | T/C |
| 5 | 40501171 | A/C |
| 5 | 40530590 | G/T |
| 5 | 40584062 | T/C |
| 5 | 40616022 | C/T |
| 5 | 40624025 | C/G |
| 5 | 40626970 | G/A |
| 5 | 40679002 | G/A |
| 5 | 40680695 | A/G |
| 5 | 40718710 | C/A |
| 5 | 40740575 | A/C |
| 5 | 40822749 | G/A |
| 5 | 40835479 | T/C |
| 5 | 40854844 | C/T |
| 5 | 40879848 | T/G |
| 5 | 40882027 | C/T |
| 5 | 40916596 | C/A |
| 5 | 40949027 | A/G |
| 5 | 40953669 | A/T |
| 5 | 40965262 | T/C |
| 5 | 40997278 | C/T |
| 5 | 41026520 | G/T |
| 5 | 41055310 | T/C |
| 5 | 41079300 | C/G |
| 5 | 41122537 | G/A |
| 5 | 41140209 | A/G |
| 5 | 41161748 | G/A |
| 5 | 41186220 | G/A |
| 5 | 41192658 | T/G |
| 5 | 41244668 | A/G |
| 5 | 41247028 | C/T |
| 5 | 41251957 | C/A |
| 5 | 41253209 | A/T |
| 5 | 41289685 | A/G |
| 5 | 41295940 | T/G |
| 5 | 41323453 | A/G |
| 5 | 41337321 | A/T |
| 5 | 41362211 | A/C |
| 5 | 41364086 | A/T |
| 5 | 41371589 | A/G |
| 5 | 41402438 | T/C |
| 5 | 41426690 | T/C |
| 5 | 41433225 | T/C |
| 5 | 41464189 | A/G |
| 5 | 41490031 | C/T |
| 5 | 41549903 | T/C |
| 5 | 41553519 | A/G |
| 5 | 41580129 | G/A |
| 5 | 41615338 | G/A |
| 5 | 41632911 | C/T |
| 5 | 41645797 | G/T |
| 5 | 41718523 | C/G |
| 5 | 41727392 | A/T |
| 5 | 41766794 | C/T |
| 5 | 41792081 | G/A |
| 5 | 41816356 | G/A |
| 5 | 41833244 | T/C |
| 5 | 41870342 | T/C |
| 5 | 41892332 | C/T |
| 5 | 41927675 | A/C |
| 5 | 41975628 | T/C |
| 5 | 41977657 | T/G |
| 5 | 42006841 | C/T |
| 5 | 42032944 | G/A |
| 5 | 42049291 | G/A |
| 5 | 42136492 | G/C |
| 5 | 42147646 | T/C |
| 5 | 42156201 | G/A |
| 5 | 42262962 | G/A |
| 5 | 42311712 | T/C |
| 5 | 42340512 | C/T |
| 6 | 5243 | A/G |
| 6 | 48648 | T/G |
| 6 | 53334 | C/T |
| 6 | 69818 | T/A |
| 6 | 82298 | A/G |
| 6 | 106802 | G/A |
| 6 | 141702 | G/A |
| 6 | 171048 | T/C |
| 6 | 176007 | A/T |
| 6 | 186505 | C/T |
| 6 | 226440 | G/C |
| 6 | 256364 | C/T |
| 6 | 262863 | G/C |
| 6 | 282607 | T/C |
| 6 | 291090 | A/T |
| 6 | 308155 | C/T |
| 6 | 336918 | A/C |
| 6 | 375671 | T/C |
| 6 | 381876 | C/G |
| 6 | 394934 | C/T |
| 6 | 422098 | G/C |
| 6 | 427796 | G/A |
| 6 | 457777 | T/C |
| 6 | 469452 | C/T |
| 6 | 480825 | T/G |
| 6 | 483055 | A/G |
| 6 | 520535 | G/A |
| 6 | 534845 | T/C |
| 6 | 541142 | T/C |
| 6 | 553294 | G/C |
| 6 | 588149 | T/C |
| 6 | 621266 | G/A |
| 6 | 625888 | A/G |
| 6 | 636089 | T/C |
| 6 | 637359 | G/A |
| 6 | 662501 | C/T |
| 6 | 678816 | C/T |
| 6 | 691379 | G/C |
| 6 | 727998 | G/A |
| 6 | 732846 | A/G |
| 6 | 756556 | G/C |
| 6 | 761101 | A/G |
| 6 | 787820 | T/A |
| 6 | 802854 | T/G |
| 6 | 828614 | C/T |
| 6 | 877917 | A/C |
| 6 | 879476 | T/C |
| 6 | 918045 | A/G |
| 6 | 931201 | C/T |
| 6 | 964639 | T/C |
| 6 | 1032440 | T/C |
| 6 | 1049511 | C/T |
| 6 | 1103687 | C/T |
| 6 | 1190683 | G/A |
| 6 | 1222279 | G/A |
| 6 | 1298142 | T/G |
| 6 | 1323700 | T/C |
| 6 | 1337781 | G/A |
| 6 | 1406231 | G/A |
| 6 | 1414376 | T/C |
| 6 | 1420865 | G/A |
| 6 | 1460601 | A/G |
| 6 | 1482053 | T/C |
| 6 | 1485730 | G/A |
| 6 | 1503677 | A/T |
| 6 | 1548674 | C/T |
| 6 | 1563397 | G/A |
| 6 | 1601543 | C/T |
| 6 | 1603054 | C/T |
| 6 | 1620362 | G/A |
| 6 | 1664302 | C/G |
| 6 | 1672479 | C/T |
| 6 | 1672503 | T/C |
| 6 | 1690465 | A/C |
| 6 | 1695269 | A/T |
| 6 | 1711806 | A/C |
| 6 | 1740283 | G/A |
| 6 | 1768594 | T/G |
| 6 | 1775602 | T/G |
| 6 | 1802212 | G/A |
| 6 | 1828101 | C/T |
| 6 | 1830835 | A/C |
| 6 | 1865900 | G/A |
| 6 | 1909344 | T/C |
| 6 | 1926854 | A/T |
| 6 | 1962455 | G/A |
| 6 | 1988631 | C/T |
| 6 | 1991499 | C/T |
| 6 | 2016831 | C/T |
| 6 | 2023519 | T/A |
| 6 | 2044229 | A/T |
| 6 | 2058884 | C/T |
| 6 | 2078879 | T/A |
| 6 | 2083468 | A/G |
| 6 | 2100298 | G/A |
| 6 | 2122272 | C/T |
| 6 | 2150256 | A/G |
| 6 | 2184060 | C/T |
| 6 | 2195030 | G/T |
| 6 | 2243132 | T/C |
| 6 | 2254114 | C/G |
| 6 | 2271905 | G/A |
| 6 | 2294785 | T/A |
| 6 | 2310083 | C/T |
| 6 | 2334003 | A/C |
| 6 | 2369470 | A/C |
| 6 | 2375913 | C/T |
| 6 | 2377139 | G/A |
| 6 | 2415304 | T/C |
| 6 | 2427269 | C/G |
| 6 | 2449959 | T/C |
| 6 | 2539105 | T/C |
| 6 | 2547477 | G/A |
| 6 | 2568893 | C/T |
| 6 | 2586873 | C/T |
| 6 | 2599461 | C/T |
| 6 | 2620126 | A/G |
| 6 | 2643630 | G/A |
| 6 | 2684905 | T/C |
| 6 | 2725925 | C/A |
| 6 | 2747817 | C/T |
| 6 | 2762324 | C/T |
| 6 | 2831867 | T/C |
| 6 | 2873000 | G/T |
| 6 | 2881454 | C/A |
| 6 | 2888941 | G/A |
| 6 | 2917389 | T/C |
| 6 | 2940172 | A/G |
| 6 | 3012693 | G/T |
| 6 | 3039092 | A/G |
| 6 | 3089360 | G/C |
| 6 | 3102958 | C/A |
| 6 | 3189516 | A/G |
| 6 | 3200595 | T/C |
| 6 | 3233777 | A/T |
| 6 | 3238167 | T/G |
| 6 | 3244783 | T/C |
| 6 | 3263461 | A/G |
| 6 | 3295789 | G/T |
| 6 | 3300786 | T/C |
| 6 | 3340658 | A/G |
| 6 | 3360045 | G/A |
| 6 | 3389089 | G/C |
| 6 | 3394113 | T/C |
| 6 | 3435156 | G/A |
| 6 | 3463815 | A/C |
| 6 | 3485189 | G/A |
| 6 | 3507209 | T/G |
| 6 | 3517586 | T/C |
| 6 | 3559773 | G/C |
| 6 | 3573547 | C/A |
| 6 | 3605532 | A/G |
| 6 | 3655449 | A/G |
| 6 | 3655450 | C/T |
| 6 | 3655451 | C/T |
| 6 | 3655452 | C/T |
| 6 | 3690287 | A/G |
| 6 | 3738320 | C/A |
| 6 | 3752488 | G/C |
| 6 | 3828070 | C/A |
| 6 | 3843921 | G/A |
| 6 | 3858846 | C/G |
| 6 | 3879890 | G/T |
| 6 | 3890705 | C/T |
| 6 | 3890706 | C/T |
| 6 | 3890707 | C/T |
| 6 | 3890708 | C/T |
| 6 | 3962366 | T/C |
| 6 | 3963380 | G/A |
| 6 | 3998630 | C/G |
| 6 | 4021384 | A/G |
| 6 | 4073767 | A/G |
| 6 | 4089203 | C/T |
| 6 | 4143375 | G/A |
| 6 | 4145568 | G/A |
| 6 | 4173795 | G/T |
| 6 | 4196228 | T/C |
| 6 | 4208407 | G/C |
| 6 | 4208408 | G/A |
| 6 | 4208409 | G/A |
| 6 | 4208410 | G/A |
| 6 | 4270992 | T/G |
| 6 | 4283338 | G/C |
| 6 | 4306716 | T/C |
| 6 | 4328561 | T/C |
| 6 | 4346701 | T/G |
| 6 | 4350221 | C/T |
| 6 | 4368033 | C/T |
| 6 | 4380838 | C/A |
| 6 | 4444059 | T/G |
| 6 | 4468050 | T/C |
| 6 | 4489526 | G/A |
| 6 | 4496018 | G/C |
| 6 | 4507035 | T/A |
| 6 | 4509067 | G/A |
| 6 | 4540800 | C/T |
| 6 | 4563080 | A/T |
| 6 | 4579879 | C/T |
| 6 | 4602507 | A/C |
| 6 | 4637625 | C/A |
| 6 | 4652189 | A/C |
| 6 | 4656891 | G/A |
| 6 | 4701395 | T/G |
| 6 | 4713420 | G/A |
| 6 | 4716026 | G/A |
| 6 | 4744309 | G/T |
| 6 | 4783724 | G/T |
| 6 | 4790766 | G/C |
| 6 | 4801053 | T/C |
| 6 | 4817019 | C/T |
| 6 | 4832559 | T/G |
| 6 | 4834510 | A/G |
| 6 | 4864760 | T/G |
| 6 | 4892992 | A/T |
| 6 | 4917728 | G/A |
| 6 | 4937006 | T/C |
| 6 | 4973635 | A/G |
| 6 | 4981820 | A/G |
| 6 | 4989688 | G/T |
| 6 | 5020184 | G/T |
| 6 | 5052421 | C/T |
| 6 | 5082093 | T/G |
| 6 | 5092268 | G/C |
| 6 | 5136423 | T/C |
| 6 | 5167195 | G/A |
| 6 | 5188654 | G/A |
| 6 | 5219106 | C/T |
| 6 | 5227336 | C/G |
| 6 | 5246957 | A/G |
| 6 | 5264583 | A/G |
| 6 | 5282787 | C/T |
| 6 | 5292479 | G/A |
| 6 | 5332751 | A/T |
| 6 | 5340295 | T/C |
| 6 | 5349565 | C/T |
| 6 | 5381144 | C/T |
| 6 | 5419263 | A/G |
| 6 | 5427951 | C/T |
| 6 | 5454965 | T/G |
| 6 | 5466512 | T/C |
| 6 | 5504053 | G/A |
| 6 | 5524681 | C/T |
| 6 | 5552775 | G/A |
| 6 | 5586722 | T/A |
| 6 | 5614561 | G/C |
| 6 | 5632458 | C/T |
| 6 | 5640009 | G/A |
| 6 | 5687242 | T/C |
| 6 | 5690269 | C/G |
| 6 | 5702768 | A/G |
| 6 | 5706876 | G/C |
| 6 | 5732630 | C/T |
| 6 | 5752760 | T/C |
| 6 | 5762835 | G/C |
| 6 | 5785459 | T/C |
| 6 | 5795394 | T/C |
| 6 | 5833158 | A/G |
| 6 | 5865409 | A/G |
| 6 | 5898905 | T/G |
| 6 | 5931666 | A/G |
| 6 | 5939783 | T/C |
| 6 | 5942411 | A/G |
| 6 | 5948708 | T/G |
| 6 | 5995048 | G/A |
| 6 | 6028348 | C/T |
| 6 | 6035213 | A/T |
| 6 | 6066330 | A/G |
| 6 | 6102812 | A/G |
| 6 | 6147081 | T/G |
| 6 | 6153817 | A/G |
| 6 | 6157893 | C/T |
| 6 | 6180519 | A/G |
| 6 | 6202704 | C/A |
| 6 | 6216975 | T/C |
| 6 | 6219514 | G/C |
| 6 | 6253459 | G/A |
| 6 | 6267500 | C/T |
| 6 | 6320719 | A/T |
| 6 | 6327392 | G/A |
| 6 | 6344996 | T/C |
| 6 | 6373654 | G/A |
| 6 | 6390057 | A/G |
| 6 | 6420804 | A/G |
| 6 | 6440673 | T/C |
| 6 | 6458995 | T/G |
| 6 | 6462023 | C/A |
| 6 | 6494376 | G/C |
| 6 | 6519531 | C/A |
| 6 | 6533070 | T/A |
| 6 | 6540692 | C/A |
| 6 | 6541920 | T/C |
| 6 | 6607262 | T/G |
| 6 | 6658163 | C/A |
| 6 | 6678139 | T/A |
| 6 | 6692807 | A/T |
| 6 | 6708301 | A/G |
| 6 | 6733593 | T/C |
| 6 | 6772459 | T/C |
| 6 | 6795780 | A/G |
| 6 | 6818030 | A/T |
| 6 | 6826006 | T/C |
| 6 | 6849107 | G/A |
| 6 | 6883540 | A/G |
| 6 | 6978180 | A/T |
| 6 | 7043384 | A/G |
| 6 | 7079979 | A/G |
| 6 | 7089571 | G/A |
| 6 | 7116476 | G/A |
| 6 | 7142107 | G/A |
| 6 | 7164722 | T/A |
| 6 | 7187451 | T/C |
| 6 | 7210731 | G/A |
| 6 | 7237266 | G/A |
| 6 | 7264091 | G/A |
| 6 | 7310237 | C/T |
| 6 | 7316265 | C/A |
| 6 | 7345849 | C/T |
| 6 | 7363403 | T/C |
| 6 | 7386973 | G/A |
| 6 | 7403865 | C/G |
| 6 | 7410676 | C/G |
| 6 | 7469231 | G/A |
| 6 | 7470779 | A/G |
| 6 | 7478155 | T/G |
| 6 | 7505990 | C/T |
| 6 | 7508210 | C/T |
| 6 | 7531046 | G/A |
| 6 | 7551672 | A/G |
| 6 | 7589214 | C/T |
| 6 | 7599851 | C/T |
| 6 | 7618565 | G/A |
| 6 | 7620521 | C/T |
| 6 | 7628241 | G/A |
| 6 | 7670343 | C/G |
| 6 | 7685852 | G/A |
| 6 | 7739728 | G/A |
| 6 | 7771277 | T/C |
| 6 | 7787385 | G/A |
| 6 | 7812881 | T/G |
| 6 | 7825261 | T/C |
| 6 | 7847344 | C/T |
| 6 | 7874858 | T/A |
| 6 | 7893801 | G/A |
| 6 | 7931135 | A/G |
| 6 | 8027503 | G/A |
| 6 | 8042580 | A/G |
| 6 | 8067840 | C/T |
| 6 | 8088337 | C/T |
| 6 | 8204418 | G/A |
| 6 | 8237298 | T/C |
| 6 | 8272517 | A/C |
| 6 | 8274217 | C/G |
| 6 | 8338204 | G/A |
| 6 | 8343644 | C/T |
| 6 | 8367793 | C/T |
| 6 | 8398834 | A/G |
| 6 | 8433418 | C/T |
| 6 | 8464556 | G/A |
| 6 | 8497632 | C/T |
| 6 | 8560380 | G/A |
| 6 | 8572811 | G/A |
| 6 | 8586400 | G/A |
| 6 | 8615808 | C/A |
| 6 | 8655121 | C/T |
| 6 | 8661071 | G/A |
| 6 | 8699841 | G/A |
| 6 | 8702540 | G/A |
| 6 | 8731331 | A/G |
| 6 | 8743730 | C/T |
| 6 | 8769511 | C/T |
| 6 | 8795750 | G/C |
| 6 | 8808047 | C/T |
| 6 | 8835469 | G/T |
| 6 | 8852839 | G/A |
| 6 | 8904586 | G/A |
| 6 | 8934753 | G/T |
| 6 | 8947071 | C/T |
| 6 | 8952814 | C/T |
| 6 | 8994932 | C/T |
| 6 | 9019262 | CT |
| 6 | 9135708 | G/A |
| 6 | 9151942 | A/T |
| 6 | 9174368 | G/C |
| 6 | 9196622 | G/A |
| 6 | 9224548 | A/C |
| 6 | 9233965 | G/A |
| 6 | 9257937 | T/C |
| 6 | 9281764 | C/G |
| 6 | 9287352 | T/C |
| 6 | 9314085 | A/G |
| 6 | 9322688 | C/T |
| 6 | 9348700 | G/A |
| 6 | 9386079 | A/T |
| 6 | 9429626 | G/A |
| 6 | 9443851 | A/G |
| 6 | 9493092 | T/G |
| 6 | 9496560 | C/T |
| 6 | 9535655 | A/G |
| 6 | 9552026 | G/A |
| 6 | 9574266 | A/G |
| 6 | 9586328 | A/G |
| 6 | 9600839 | G/A |
| 6 | 9630397 | G/T |
| 6 | 9679155 | T/C |
| 6 | 9695477 | A/T |
| 6 | 9697737 | C/T |
| 6 | 9722072 | C/T |
| 6 | 9736572 | A/G |
| 6 | 9820538 | T/C |
| 6 | 9833984 | C/G |
| 6 | 9851919 | G/T |
| 6 | 9894344 | T/A |
| 6 | 9918487 | T/C |
| 6 | 9951025 | A/G |
| 6 | 9992471 | T/C |
| 6 | 10033663 | T/C |
| 6 | 10130680 | C/T |
| 6 | 10137997 | C/T |
| 6 | 10174319 | C/A |
| 6 | 10236910 | T/C |
| 6 | 10292672 | A/G |
| 6 | 10324084 | T/C |
| 6 | 10363252 | T/C |
| 6 | 10390470 | T/C |
| 6 | 10452594 | A/G |
| 6 | 10457720 | C/T |
| 6 | 10486126 | T/C |
| 6 | 10515471 | G/A |
| 6 | 10535452 | A/G |
| 6 | 10540763 | C/T |
| 6 | 10577475 | G/A |
| 6 | 10597550 | C/T |
| 6 | 10638803 | C/A |
| 6 | 10657055 | C/T |
| 6 | 10678043 | A/C |
| 6 | 10740166 | A/G |
| 6 | 10755506 | G/A |
| 6 | 10814224 | G/A |
| 6 | 10821059 | G/A |
| 6 | 10840255 | C/T |
| 6 | 10874732 | C/T |
| 6 | 10919574 | A/G |
| 6 | 10943313 | C/T |
| 6 | 10968205 | G/C |
| 6 | 11039830 | C/T |
| 6 | 11044762 | C/T |
| 6 | 11078289 | C/T |
| 6 | 11102209 | T/G |
| 6 | 11144303 | T/A |
| 6 | 11153814 | G/C |
| 6 | 11161443 | C/T |
| 6 | 11191934 | A/G |
| 6 | 11224280 | C/T |
| 6 | 11241259 | C/T |
| 6 | 11256102 | G/A |
| 6 | 11263433 | T/G |
| 6 | 11305256 | T/C |
| 6 | 11308268 | C/T |
| 6 | 11311325 | C/T |
| 6 | 11321615 | G/T |
| 6 | 11352581 | A/G |
| 6 | 11394711 | T/C |
| 6 | 11419420 | C/T |
| 6 | 11461597 | C/T |
| 6 | 11493986 | G/A |
| 6 | 11501205 | G/A |
| 6 | 11534293 | T/A |
| 6 | 11558978 | C/T |
| 6 | 11560915 | T/G |
| 6 | 11583855 | T/C |
| 6 | 11622364 | A/G |
| 6 | 11653228 | A/T |
| 6 | 11669926 | A/G |
| 6 | 11685171 | C/G |
| 6 | 11708946 | C/T |
| 6 | 11732541 | G/A |
| 6 | 11742603 | A/C |
| 6 | 11794139 | C/T |
| 6 | 11819560 | C/T |
| 6 | 11829879 | G/A |
| 6 | 11843250 | C/T |
| 6 | 11873334 | A/T |
| 6 | 11879204 | G/T |
| 6 | 11909961 | G/A |
| 6 | 11938541 | G/A |
| 6 | 11965751 | G/A |
| 6 | 11973271 | G/A |
| 6 | 11989293 | G/A |
| 6 | 12049943 | A/G |
| 6 | 12060332 | G/A |
| 6 | 12103701 | G/A |
| 6 | 12104075 | G/C |
| 6 | 12104916 | T/G |
| 6 | 12105054 | G/A |
| 6 | 12127292 | G/A |
| 6 | 12138107 | A/G |
| 6 | 12158369 | G/A |
| 6 | 12163822 | G/T |
| 6 | 12208789 | C/T |
| 6 | 12210581 | T/C |
| 6 | 12233297 | G/A |
| 6 | 12252388 | C/G |
| 6 | 12255745 | C/T |
| 6 | 12281595 | A/G |
| 6 | 12291257 | C/T |
| 6 | 12303869 | C/T |
| 6 | 12306690 | T/G |
| 6 | 12373300 | C/A |
| 6 | 12393181 | C/T |
| 6 | 12467848 | G/A |
| 6 | 12467849 | A/G |
| 6 | 12467850 | A/G |
| 6 | 12467851 | A/G |
| 6 | 12581557 | G/A |
| 6 | 12583868 | G/A |
| 6 | 12628192 | A/G |
| 6 | 12646429 | C/G |
| 6 | 12657501 | A/G |
| 6 | 12676903 | C/T |
| 6 | 12680505 | A/C |
| 6 | 12693369 | G/A |
| 6 | 12722070 | C/G |
| 6 | 12729898 | C/T |
| 6 | 12773509 | C/G |
| 6 | 12783542 | G/A |
| 6 | 12811175 | C/T |
| 6 | 12842271 | G/A |
| 6 | 12921601 | C/T |
| 6 | 12943538 | G/C |
| 6 | 13013202 | C/T |
| 6 | 13048935 | G/A |
| 6 | 13059348 | T/C |
| 6 | 13115907 | A/C |
| 6 | 13142189 | C/T |
| 6 | 13183986 | C/T |
| 6 | 13207449 | T/G |
| 6 | 13311540 | A/G |
| 6 | 13326416 | C/G |
| 6 | 13477898 | G/A |
| 6 | 13520131 | A/G |
| 6 | 13625076 | T/G |
| 6 | 13643908 | G/A |
| 6 | 13665645 | A/G |
| 6 | 13669013 | G/T |
| 6 | 13759264 | C/T |
| 6 | 13765281 | C/T |
| 6 | 13821078 | T/G |
| 6 | 13839967 | G/A |
| 6 | 13877546 | G/A |
| 6 | 13910240 | G/A |
| 6 | 13912353 | C/T |
| 6 | 13923934 | C/A |
| 6 | 13978693 | T/C |
| 6 | 14150192 | G/A |
| 6 | 14175855 | A/T |
| 6 | 14205985 | C/T |
| 6 | 14226807 | C/A |
| 6 | 14260218 | C/A |
| 6 | 14362374 | G/A |
| 6 | 14379919 | G/A |
| 6 | 14396119 | C/T |
| 6 | 14427224 | G/T |
| 6 | 14462661 | G/A |
| 6 | 14491926 | G/C |
| 6 | 14553867 | C/T |
| 6 | 14600711 | G/A |
| 6 | 14615280 | C/T |
| 6 | 14620118 | A/G |
| 6 | 14689687 | G/T |
| 6 | 14701352 | T/C |
| 6 | 14704595 | G/A |
| 6 | 14734874 | A/G |
| 6 | 14777461 | G/C |
| 6 | 14801291 | C/T |
| 6 | 14837158 | G/A |
| 6 | 14858663 | G/A |
| 6 | 14927474 | G/A |
| 6 | 14928943 | G/A |
| 6 | 14946467 | G/A |
| 6 | 14952033 | C/T |
| 6 | 14996479 | C/G |
| 6 | 15054650 | C/T |
| 6 | 15080181 | G/A |
| 6 | 15101958 | C/T |
| 6 | 15119330 | C/T |
| 6 | 15128135 | G/A |
| 6 | 15179209 | C/A |
| 6 | 15202826 | A/G |
| 6 | 15228150 | C/G |
| 6 | 15259764 | C/T |
| 6 | 15264275 | T/C |
| 6 | 15291882 | T/A |
| 6 | 15299111 | C/T |
| 6 | 15313078 | T/C |
| 6 | 15322209 | A/G |
| 6 | 15337463 | T/C |
| 6 | 15356406 | G/T |
| 6 | 15361968 | G/A |
| 6 | 15407701 | A/G |
| 6 | 15412180 | C/T |
| 6 | 15434191 | C/T |
| 6 | 15456509 | C/T |
| 6 | 15488675 | G/C |
| 6 | 15524884 | G/C |
| 6 | 15533922 | A/T |
| 6 | 15560870 | C/T |
| 6 | 15570186 | T/C |
| 6 | 15573272 | T/C |
| 6 | 15614856 | A/G |
| 6 | 15666852 | C/T |
| 6 | 15680146 | C/T |
| 6 | 15695337 | G/C |
| 6 | 15729741 | T/A |
| 6 | 15742683 | T/G |
| 6 | 15775638 | C/T |
| 6 | 15919396 | G/A |
| 6 | 15926242 | A/C |
| 6 | 15930920 | G/T |
| 6 | 15977335 | G/A |
| 6 | 15992262 | C/T |
| 6 | 15996557 | A/T |
| 6 | 16028398 | A/G |
| 6 | 16060235 | G/A |
| 6 | 16078411 | A/T |
| 6 | 16082408 | C/G |
| 6 | 16104074 | G/A |
| 6 | 16111664 | C/T |
| 6 | 16134702 | G/T |
| 6 | 16142659 | A/G |
| 6 | 16190341 | C/T |
| 6 | 16205512 | A/G |
| 6 | 16256691 | T/C |
| 6 | 16279147 | G/A |
| 6 | 16294622 | C/T |
| 6 | 16328613 | C/T |
| 6 | 16335554 | G/A |
| 6 | 16414246 | A/G |
| 6 | 16431798 | T/C |
| 6 | 16449790 | T/G |
| 6 | 16467867 | C/T |
| 6 | 16477384 | A/G |
| 6 | 16496365 | T/C |
| 6 | 16507428 | C/G |
| 6 | 16554911 | A/G |
| 6 | 16568593 | C/G |
| 6 | 16600254 | T/C |
| 6 | 16608805 | T/G |
| 6 | 16630598 | G/A |
| 6 | 16660041 | C/G |
| 6 | 16696838 | T/C |
| 6 | 16707525 | G/A |
| 6 | 16718048 | G/A |
| 6 | 16726989 | C/T |
| 6 | 16740498 | C/G |
| 6 | 16768992 | A/T |
| 6 | 16773268 | A/T |
| 6 | 16792178 | C/G |
| 6 | 16802879 | A/T |
| 6 | 16815926 | G/C |
| 6 | 16856190 | T/C |
| 6 | 16869359 | C/T |
| 6 | 16895637 | G/A |
| 6 | 16915973 | A/G |
| 6 | 16945385 | C/T |
| 6 | 17009887 | C/T |
| 6 | 17013620 | A/G |
| 6 | 17047155 | T/A |
| 6 | 17097349 | C/G |
| 6 | 17105297 | T/A |
| 6 | 17138345 | C/T |
| 6 | 17162069 | A/T |
| 6 | 17172847 | C/T |
| 6 | 17218830 | G/A |
| 6 | 17244217 | C/G |
| 6 | 17246615 | T/C |
| 6 | 17261696 | G/A |
| 6 | 17336372 | C/T |
| 6 | 17337546 | T/G |
| 6 | 17344109 | C/T |
| 6 | 17359521 | C/A |
| 6 | 17389284 | G/T |
| 6 | 17402013 | G/A |
| 6 | 17473563 | C/T |
| 6 | 17518968 | G/A |
| 6 | 17528824 | A/G |
| 6 | 17533045 | C/T |
| 6 | 17559035 | T/C |
| 6 | 17587609 | C/T |
| 6 | 17616443 | C/T |
| 6 | 17626754 | G/A |
| 6 | 17708277 | G/A |
| 6 | 17716189 | C/T |
| 6 | 17738491 | A/G |
| 6 | 17788274 | G/A |
| 6 | 17792214 | G/A |
| 6 | 17798541 | G/A |
| 6 | 17824192 | G/A |
| 6 | 17861556 | A/T |
| 6 | 17867720 | C/A |
| 6 | 17891933 | A/G |
| 6 | 17895965 | T/C |
| 6 | 17924926 | G/A |
| 6 | 17942980 | C/T |
| 6 | 18149442 | C/A |
| 6 | 18329049 | G/A |
| 6 | 18342426 | C/T |
| 6 | 18350459 | C/T |
| 6 | 18428093 | C/T |
| 6 | 18441896 | G/A |
| 6 | 18450742 | C/T |
| 6 | 18469106 | G/A |
| 6 | 18486752 | C/A |
| 6 | 18499816 | G/A |
| 6 | 18518060 | A/G |
| 6 | 18534129 | C/T |
| 6 | 18560803 | C/T |
| 6 | 18581452 | G/C |
| 6 | 18614800 | C/T |
| 6 | 18669334 | T/C |
| 6 | 18687279 | G/A |
| 6 | 18715387 | A/G |
| 6 | 18740900 | A/C |
| 6 | 18744106 | G/T |
| 6 | 18757845 | G/A |
| 6 | 18781527 | A/G |
| 6 | 18817329 | A/G |
| 6 | 18821479 | C/T |
| 6 | 18852301 | A/G |
| 6 | 18855450 | A/G |
| 6 | 18881266 | A/G |
| 6 | 18899924 | A/G |
| 6 | 18901799 | C/T |
| 6 | 18953186 | A/G |
| 6 | 18959098 | T/A |
| 6 | 18962063 | A/C |
| 6 | 19013079 | C/T |
| 6 | 19040948 | A/T |
| 6 | 19075202 | T/C |
| 6 | 19082619 | G/T |
| 6 | 19151768 | T/C |
| 6 | 19170068 | T/C |
| 6 | 19182222 | C/A |
| 6 | 19226105 | C/A |
| 6 | 19236257 | G/T |
| 6 | 19276260 | C/T |
| 6 | 19286380 | T/A |
| 6 | 19299353 | G/A |
| 6 | 19304002 | C/G |
| 6 | 19333138 | A/T |
| 6 | 19347223 | T/G |
| 6 | 19350321 | G/T |
| 6 | 19361710 | C/A |
| 6 | 19394322 | A/T |
| 6 | 19402609 | G/A |
| 6 | 19410511 | C/T |
| 6 | 19432200 | T/C |
| 6 | 19458282 | G/A |
| 6 | 19473209 | T/G |
| 6 | 19492101 | T/C |
| 6 | 19502270 | T/C |
| 6 | 19509052 | T/C |
| 6 | 19546009 | G/A |
| 6 | 19576313 | G/A |
| 6 | 19596378 | C/T |
| 6 | 19597927 | T/C |
| 6 | 19638100 | T/C |
| 6 | 19656144 | A/T |
| 6 | 19706573 | C/T |
| 6 | 19717635 | A/G |
| 6 | 19736461 | T/C |
| 6 | 19768186 | C/G |
| 6 | 19782775 | T/A |
| 6 | 19802092 | T/C |
| 6 | 19837756 | C/T |
| 6 | 19845246 | C/T |
| 6 | 19876705 | G/A |
| 6 | 19877780 | G/C |
| 6 | 19893648 | C/A |
| 6 | 19938846 | G/A |
| 6 | 19956007 | T/C |
| 6 | 19986978 | G/A |
| 6 | 20006381 | C/T |
| 6 | 20011035 | C/G |
| 6 | 20028263 | T/G |
| 6 | 20073703 | G/A |
| 6 | 20098807 | C/G |
| 6 | 20102388 | C/T |
| 6 | 20114157 | A/C |
| 6 | 20132028 | A/G |
| 6 | 20157477 | T/C |
| 6 | 20180689 | A/G |
| 6 | 20208994 | C/T |
| 6 | 20232259 | C/A |
| 6 | 20239946 | G/A |
| 6 | 20254096 | A/G |
| 6 | 20263050 | C/T |
| 6 | 20282937 | A/G |
| 6 | 20308458 | C/G |
| 6 | 20312185 | G/A |
| 6 | 20336581 | T/C |
| 6 | 20347726 | T/C |
| 6 | 20353346 | G/A |
| 6 | 20380800 | A/C |
| 6 | 20398211 | A/G |
| 6 | 20403437 | C/T |
| 6 | 20465517 | C/T |
| 6 | 20468200 | G/A |
| 6 | 20517227 | T/A |
| 6 | 20521361 | G/A |
| 6 | 20537526 | G/A |
| 6 | 20550574 | C/T |
| 6 | 20601287 | G/T |
| 6 | 20602835 | C/T |
| 6 | 20617868 | G/A |
| 6 | 20620731 | T/C |
| 6 | 20667740 | C/T |
| 6 | 20670080 | C/T |
| 6 | 20681897 | T/A |
| 6 | 20710826 | T/C |
| 6 | 20729420 | C/T |
| 6 | 20759192 | T/A |
| 6 | 20776755 | A/G |
| 6 | 20784699 | T/C |
| 6 | 20800708 | A/G |
| 6 | 20844193 | G/A |
| 6 | 20849214 | C/T |
| 6 | 20859669 | C/A |
| 6 | 20868972 | T/C |
| 6 | 20888318 | A/G |
| 6 | 20916378 | G/A |
| 6 | 20930666 | A/G |
| 6 | 20965488 | G/A |
| 6 | 20985010 | C/T |
| 6 | 20996002 | G/T |
| 6 | 21008954 | T/G |
| 6 | 21058433 | G/C |
| 6 | 21064621 | C/T |
| 6 | 21088373 | A/C |
| 6 | 21132548 | T/C |
| 6 | 21154602 | G/A |
| 6 | 21189972 | C/T |
| 6 | 21193309 | G/C |
| 6 | 21227756 | C/G |
| 6 | 21234258 | C/T |
| 6 | 21241731 | G/A |
| 6 | 21291045 | T/C |
| 6 | 21320512 | C/T |
| 6 | 21324369 | G/A |
| 6 | 21340320 | C/A |
| 6 | 21362731 | G/C |
| 6 | 21374083 | C/A |
| 6 | 21393249 | G/A |
| 6 | 21394831 | A/G |
| 6 | 21420996 | G/A |
| 6 | 21458778 | T/G |
| 6 | 21496176 | G/A |
| 6 | 21523644 | C/T |
| 6 | 21570616 | A/G |
| 6 | 21583114 | A/G |
| 6 | 21615884 | A/G |
| 6 | 21637253 | C/T |
| 6 | 21660420 | A/G |
| 6 | 21683735 | G/A |
| 6 | 21726365 | T/C |
| 6 | 21728097 | C/T |
| 6 | 21775402 | G/A |
| 6 | 21801662 | G/A |
| 6 | 21819734 | T/C |
| 6 | 21836283 | C/T |
| 6 | 21928213 | A/G |
| 6 | 21952563 | G/A |
| 6 | 21989511 | G/A |
| 6 | 22003099 | T/A |
| 6 | 22047459 | A/G |
| 6 | 22055978 | T/C |
| 6 | 22074953 | T/C |
| 6 | 22088846 | A/G |
| 6 | 22104901 | C/T |
| 6 | 22154356 | C/T |
| 6 | 22188318 | C/T |
| 6 | 22208324 | A/T |
| 6 | 22233069 | C/T |
| 6 | 22313575 | C/T |
| 6 | 22348340 | G/A |
| 6 | 22351000 | C/T |
| 6 | 22374391 | G/A |
| 6 | 22381122 | G/A |
| 6 | 22389929 | C/T |
| 6 | 22423155 | C/T |
| 6 | 22434186 | C/T |
| 6 | 22445112 | A/G |
| 6 | 22489769 | G/A |
| 6 | 22521798 | T/C |
| 6 | 22526464 | G/C |
| 6 | 22579870 | C/T |
| 6 | 22581223 | C/A |
| 6 | 22599520 | A/G |
| 6 | 22639635 | G/A |
| 6 | 22653854 | T/C |
| 6 | 22682712 | C/A |
| 6 | 22685188 | C/T |
| 6 | 22710318 | A/T |
| 6 | 22747823 | A/C |
| 6 | 22788625 | A/T |
| 6 | 22808801 | C/G |
| 6 | 22826745 | T/C |
| 6 | 22848787 | T/C |
| 6 | 22887825 | A/G |
| 6 | 22901382 | T/C |
| 6 | 22928683 | C/T |
| 6 | 22935217 | G/A |
| 6 | 22961250 | G/A |
| 6 | 22967638 | C/A |
| 6 | 22995042 | T/C |
| 6 | 23004737 | A/G |
| 6 | 23014500 | A/G |
| 6 | 23022559 | C/T |
| 6 | 23043006 | C/T |
| 6 | 23068164 | C/T |
| 6 | 23090806 | T/G |
| 6 | 23093219 | G/A |
| 6 | 23121476 | G/A |
| 6 | 23124790 | A/G |
| 6 | 23137068 | T/C |
| 6 | 23139411 | G/A |
| 6 | 23180126 | C/G |
| 6 | 23214872 | C/A |
| 6 | 23235708 | C/T |
| 6 | 23242351 | A/C |
| 6 | 23237617 | T/C |
| 6 | 23270211 | T/C |
| 6 | 23292778 | G/A |
| 6 | 23309434 | T/C |
| 6 | 23323703 | A/G |
| 6 | 23324877 | C/T |
| 6 | 23363811 | G/C |
| 6 | 23379509 | G/T |
| 6 | 23411610 | G/A |
| 6 | 23420308 | G/T |
| 6 | 23432212 | G/A |
| 6 | 23502834 | G/A |
| 6 | 23545974 | G/A |
| 6 | 23565528 | G/A |
| 6 | 23571602 | A/G |
| 6 | 23599045 | C/T |
| 6 | 23619969 | T/C |
| 6 | 23656840 | T/G |
| 6 | 23670271 | A/G |
| 6 | 23676259 | G/A |
| 6 | 23709082 | A/G |
| 6 | 23714064 | A/G |
| 6 | 23732195 | G/A |
| 6 | 23760108 | T/C |
| 6 | 23773719 | A/C |
| 6 | 23796341 | G/A |
| 6 | 23799592 | T/C |
| 6 | 23830230 | T/A |
| 6 | 23834687 | C/G |
| 6 | 23872980 | C/A |
| 6 | 23895295 | C/A |
| 6 | 23902703 | A/G |
| 6 | 23928190 | C/T |
| 6 | 23938768 | C/A |
| 6 | 23941951 | T/C |
| 6 | 23965340 | C/T |
| 6 | 24025178 | G/A |
| 6 | 24059307 | A/G |
| 6 | 24076017 | A/C |
| 6 | 24095127 | G/A |
| 6 | 24103797 | G/A |
| 6 | 24163587 | C/T |
| 6 | 24191119 | C/A |
| 6 | 24215902 | C/T |
| 6 | 24239957 | G/A |
| 6 | 24265709 | G/A |
| 6 | 24295432 | C/T |
| 6 | 24311532 | G/A |
| 6 | 24320438 | G/A |
| 6 | 24351882 | T/C |
| 6 | 24359838 | T/C |
| 6 | 24380577 | A/G |
| 6 | 24407340 | C/T |
| 6 | 24432623 | C/A |
| 6 | 24437446 | T/C |
| 6 | 24461716 | G/A |
| 6 | 24463976 | C/T |
| 6 | 24487345 | A/T |
| 6 | 24550700 | C/T |
| 6 | 24600155 | T/C |
| 6 | 24651066 | G/A |
| 6 | 24687890 | A/T |
| 6 | 24697298 | C/T |
| 6 | 24783513 | T/C |
| 6 | 24792205 | C/T |
| 6 | 24818397 | A/G |
| 6 | 24903249 | G/A |
| 6 | 24928110 | C/T |
| 6 | 24932853 | G/A |
| 6 | 25003287 | C/A |
| 6 | 25055651 | A/G |
| 6 | 25081474 | G/A |
| 6 | 25242699 | T/G |
| 6 | 25267189 | G/C |
| 6 | 25278548 | C/T |
| 6 | 25292341 | C/T |
| 6 | 25294063 | A/G |
| 6 | 25322083 | C/T |
| 6 | 25363383 | G/A |
| 6 | 25372384 | C/A |
| 6 | 25386377 | T/G |
| 6 | 25406515 | C/T |
| 6 | 25430129 | C/T |
| 6 | 25458829 | A/G |
| 6 | 25481811 | C/A |
| 6 | 25510964 | G/A |
| 6 | 25517700 | G/A |
| 6 | 25533988 | G/A |
| 6 | 25540566 | C/T |
| 6 | 25562115 | C/T |
| 6 | 25605895 | A/G |
| 6 | 25636912 | T/A |
| 6 | 25638138 | T/A |
| 6 | 25668962 | T/C |
| 6 | 25699112 | C/G |
| 6 | 25708038 | G/A |
| 6 | 25728843 | C/T |
| 6 | 25754415 | T/G |
| 6 | 25783748 | G/C |
| 6 | 25805873 | T/C |
| 6 | 25809468 | G/A |
| 6 | 25855774 | G/A |
| 6 | 25862182 | C/T |
| 6 | 25891430 | G/A |
| 6 | 25906973 | G/A |
| 6 | 25964788 | T/G |
| 6 | 25971713 | A/T |
| 6 | 25983265 | C/T |
| 6 | 25992756 | G/A |
| 6 | 26014350 | C/T |
| 6 | 26022721 | C/T |
| 6 | 26055962 | G/A |
| 6 | 26065845 | T/C |
| 6 | 26090508 | T/C |
| 6 | 26100826 | A/G |
| 6 | 26137111 | T/C |
| 6 | 26180078 | T/G |
| 6 | 26209665 | C/A |
| 6 | 26224953 | T/C |
| 6 | 26228002 | T/C |
| 6 | 26253208 | C/A |
| 6 | 26293963 | G/C |
| 6 | 26318852 | G/A |
| 6 | 26320304 | C/T |
| 6 | 26369560 | A/G |
| 6 | 26376817 | C/G |
| 6 | 26396732 | A/C |
| 6 | 26400845 | A/G |
| 6 | 26451318 | A/T |
| 6 | 26456110 | T/G |
| 6 | 26468387 | A/C |
| 6 | 26482279 | T/C |
| 6 | 26506618 | C/A |
| 6 | 26512201 | A/G |
| 6 | 26520606 | T/C |
| 6 | 26549679 | T/C |
| 6 | 26557884 | A/T |
| 6 | 26577923 | G/T |
| 6 | 26592200 | C/T |
| 6 | 26607641 | G/A |
| 6 | 26611015 | C/A |
| 6 | 26616222 | C/T |
| 6 | 26641355 | A/G |
| 6 | 26656658 | G/A |
| 6 | 26697073 | G/A |
| 6 | 26701808 | C/G |
| 6 | 26712489 | G/A |
| 6 | 26733861 | C/T |
| 6 | 26733862 | T/C |
| 6 | 26733863 | T/C |
| 6 | 26733864 | T/C |
| 6 | 26816402 | C/A |
| 6 | 26820002 | G/A |
| 6 | 26826408 | A/G |
| 6 | 26858431 | G/A |
| 6 | 26875124 | G/A |
| 6 | 26883930 | G/A |
| 6 | 26890707 | C/G |
| 6 | 26926895 | A/G |
| 6 | 26935132 | C/T |
| 6 | 26947304 | C/G |
| 6 | 26949009 | G/A |
| 6 | 27005446 | A/G |
| 6 | 27015702 | A/G |
| 6 | 27035047 | G/C |
| 6 | 27062526 | A/G |
| 6 | 27077593 | G/A |
| 6 | 27101274 | T/C |
| 6 | 27209333 | A/C |
| 6 | 27220400 | T/C |
| 6 | 27242344 | A/G |
| 6 | 27261857 | C/T |
| 6 | 27292085 | C/T |
| 6 | 27312793 | G/A |
| 6 | 27355806 | A/G |
| 6 | 27373749 | C/T |
| 6 | 27378990 | T/C |
| 6 | 27394369 | G/A |
| 6 | 27434932 | G/A |
| 6 | 27441115 | G/A |
| 6 | 27451024 | C/T |
| 6 | 27457522 | C/T |
| 6 | 27481896 | A/G |
| 6 | 27538447 | G/A |
| 6 | 27566428 | G/A |
| 6 | 27591976 | A/G |
| 6 | 27595265 | C/A |
| 6 | 27601033 | A/G |
| 6 | 27616422 | T/C |
| 6 | 27650692 | C/T |
| 6 | 27662466 | T/G |
| 6 | 27700477 | G/A |
| 6 | 27795477 | T/C |
| 6 | 27913790 | G/A |
| 6 | 27918137 | G/A |
| 6 | 27953470 | C/T |
| 6 | 27971446 | G/A |
| 6 | 28002728 | G/A |
| 6 | 28036440 | G/A |
| 6 | 28038580 | C/T |
| 6 | 28094866 | C/T |
| 6 | 28127923 | C/A |
| 6 | 28167226 | C/A |
| 6 | 28175221 | C/T |
| 6 | 28176961 | G/A |
| 6 | 28200157 | G/A |
| 6 | 28212804 | C/T |
| 6 | 28284634 | A/G |
| 6 | 28291955 | G/A |
| 6 | 28364180 | C/T |
| 6 | 28378421 | G/C |
| 6 | 28387957 | A/G |
| 6 | 28412918 | C/T |
| 6 | 28421792 | T/C |
| 6 | 28445110 | G/A |
| 6 | 28457497 | A/G |
| 6 | 28475966 | A/C |
| 6 | 28482106 | A/G |
| 6 | 28510527 | C/T |
| 6 | 28525778 | T/A |
| 6 | 28532686 | C/T |
| 6 | 28540978 | C/T |
| 6 | 28569745 | A/G |
| 6 | 28602432 | T/C |
| 6 | 28622431 | C/T |
| 6 | 28655674 | G/A |
| 6 | 28682526 | C/T |
| 6 | 28730759 | A/C |
| 6 | 28765977 | A/C |
| 6 | 28787971 | T/C |
| 6 | 28874139 | C/T |
| 6 | 28934590 | A/G |
| 6 | 28945100 | G/A |
| 6 | 28964091 | C/T |
| 6 | 29011543 | T/C |
| 6 | 29042604 | A/G |
| 6 | 29090689 | C/T |
| 6 | 29128468 | A/T |
| 6 | 29167841 | A/G |
| 6 | 29190571 | G/C |
| 6 | 29207434 | A/G |
| 6 | 29241918 | A/G |
| 6 | 29270117 | C/T |
| 6 | 29291894 | C/G |
| 6 | 29368548 | G/A |
| 6 | 29387102 | C/T |
| 6 | 29401295 | G/A |
| 6 | 28409063 | C/A |
| 6 | 29433643 | C/T |
| 6 | 29459342 | C/G |
| 6 | 29478389 | C/T |
| 6 | 29541463 | G/A |
| 6 | 29557679 | C/T |
| 6 | 29580131 | C/T |
| 6 | 29615935 | C/G |
| 6 | 29633139 | A/G |
| 6 | 29657726 | T/A |
| 6 | 29721733 | C/G |
| 6 | 29787998 | G/A |
| 6 | 29795368 | G/A |
| 6 | 29814542 | C/G |
| 6 | 29832387 | T/C |
| 6 | 29855486 | T/C |
| 6 | 29876962 | C/T |
| 6 | 29891136 | G/A |
| 6 | 29906609 | A/G |
| 6 | 29914652 | C/T |
| 6 | 29942425 | C/T |
| 6 | 29945896 | G/A |
| 6 | 30014098 | G/A |
| 6 | 30032752 | G/A |
| 6 | 30086698 | C/T |
| 6 | 30114286 | G/A |
| 6 | 30116947 | G/A |
| 6 | 30132979 | T/C |
| 6 | 30164222 | T/C |
| 6 | 30176085 | T/G |
| 6 | 30257903 | C/T |
| 6 | 30278045 | T/C |
| 6 | 30281728 | T/C |
| 6 | 30313788 | C/G |
| 6 | 30329324 | T/C |
| 6 | 30354891 | C/T |
| 6 | 30368298 | A/G |
| 6 | 30394352 | G/A |
| 6 | 30408750 | A/G |
| 6 | 30441084 | C/T |
| 6 | 30472692 | G/T |
| 6 | 30497707 | G/A |
| 6 | 30522549 | C/T |
| 6 | 30566743 | G/C |
| 6 | 30587109 | G/A |
| 6 | 30603049 | C/T |
| 6 | 30614407 | C/A |
| 6 | 30640358 | G/A |
| 6 | 30663881 | G/A |
| 6 | 30684873 | T/C |
| 6 | 30692243 | G/C |
| 6 | 30719969 | C/T |
| 6 | 30730045 | C/A |
| 6 | 30764509 | C/T |
| 6 | 30784289 | T/C |
| 6 | 30830684 | A/G |
| 6 | 30849839 | C/T |
| 6 | 30906916 | G/A |
| 6 | 30951250 | C/T |
| 6 | 30953270 | A/C |
| 6 | 30976238 | T/G |
| 6 | 30985138 | G/A |
| 6 | 30994279 | C/A |
| 6 | 30996785 | T/C |
| 6 | 31039723 | G/A |
| 6 | 31050953 | G/C |
| 6 | 31064488 | G/A |
| 6 | 31108935 | G/A |
| 6 | 31127216 | A/G |
| 6 | 31164362 | A/G |
| 6 | 31185130 | G/A |
| 6 | 31200207 | A/G |
| 6 | 31236511 | C/T |
| 6 | 31267443 | G/A |
| 6 | 31274549 | G/A |
| 6 | 31327334 | T/C |
| 6 | 31361085 | G/A |
| 6 | 31393294 | C/T |
| 6 | 31424618 | T/A |
| 6 | 31497654 | T/C |
| 6 | 31501129 | C/T |
| 6 | 31529236 | C/A |
| 6 | 31549782 | C/T |
| 6 | 31589073 | A/G |
| 6 | 31595528 | T/C |
| 6 | 31600833 | A/G |
| 6 | 31666507 | G/C |
| 6 | 31678476 | A/G |
| 6 | 31688164 | T/A |
| 6 | 31725296 | C/T |
| 6 | 31746846 | G/A |
| 6 | 31788435 | G/A |
| 6 | 31804901 | C/T |
| 6 | 31837045 | G/A |
| 6 | 31849354 | G/A |
| 6 | 31921232 | C/T |
| 6 | 31926886 | T/C |
| 6 | 31953440 | C/T |
| 6 | 31972726 | T/C |
| 6 | 32045089 | A/G |
| 6 | 32108355 | A/G |
| 6 | 32156381 | C/T |
| 6 | 32179665 | G/A |
| 6 | 32184343 | G/T |
| 6 | 32191335 | A/G |
| 6 | 32220359 | C/T |
| 6 | 32282108 | C/T |
| 6 | 32290238 | G/A |
| 6 | 32328098 | G/T |
| 6 | 32343683 | C/T |
| 6 | 32362302 | G/A |
| 6 | 32384688 | G/A |
| 6 | 32392420 | T/G |
| 6 | 32429217 | A/G |
| 6 | 32439094 | C/T |
| 6 | 32453693 | C/G |
| 6 | 32461078 | C/T |
| 6 | 32499498 | T/C |
| 6 | 32513939 | A/G |
| 6 | 32529102 | C/T |
| 6 | 32549244 | C/T |
| 6 | 32571027 | G/A |
| 6 | 32581540 | T/A |
| 6 | 32609313 | T/C |
| 6 | 32648499 | C/T |
| 6 | 32669337 | C/T |
| 6 | 32673770 | T/C |
| 6 | 32712285 | A/G |
| 6 | 32724677 | A/C |
| 6 | 32738477 | A/G |
| 6 | 32818976 | C/T |
| 6 | 32828526 | T/C |
| 6 | 32866351 | C/T |
| 6 | 32871755 | T/C |
| 6 | 32892213 | C/T |
| 6 | 32899291 | T/C |
| 6 | 32915338 | A/G |
| 6 | 32951439 | T/C |
| 6 | 32962530 | C/T |
| 6 | 32972264 | G/A |
| 6 | 32978988 | T/C |
| 6 | 33004233 | T/G |
| 6 | 33039963 | C/G |
| 6 | 33069578 | T/A |
| 6 | 33071331 | A/C |
| 6 | 33106043 | G/A |
| 6 | 33119090 | A/G |
| 6 | 33121981 | G/T |
| 6 | 33147575 | G/A |
| 6 | 33171420 | C/T |
| 6 | 33172704 | T/C |
| 6 | 33193867 | G/A |
| 6 | 33210896 | C/T |
| 6 | 33232477 | C/T |
| 6 | 33256492 | A/G |
| 6 | 33267545 | C/T |
| 6 | 33286095 | G/A |
| 6 | 33304490 | A/G |
| 6 | 33329979 | C/T |
| 6 | 33357111 | G/A |
| 6 | 33376846 | T/C |
| 6 | 33406021 | C/T |
| 6 | 33415069 | G/A |
| 6 | 33428769 | T/C |
| 6 | 33448402 | T/C |
| 6 | 33456977 | C/T |
| 6 | 33466543 | G/A |
| 6 | 33502975 | G/A |
| 6 | 33506032 | A/G |
| 6 | 33525578 | C/G |
| 6 | 33556752 | C/T |
| 6 | 33558259 | A/C |
| 6 | 33571662 | G/A |
| 6 | 33573349 | A/T |
| 6 | 33600992 | G/A |
| 6 | 33618915 | T/G |
| 6 | 33647450 | C/T |
| 6 | 33654657 | C/T |
| 6 | 33680149 | C/T |
| 6 | 33686675 | G/A |
| 6 | 33717761 | T/C |
| 6 | 33722211 | G/T |
| 6 | 33760080 | T/C |
| 6 | 33791132 | C/T |
| 6 | 33816867 | T/C |
| 6 | 33835963 | T/C |
| 6 | 33847688 | C/T |
| 6 | 33872894 | C/A |
| 6 | 33878144 | G/A |
| 6 | 33911069 | G/A |
| 6 | 33925918 | G/A |
| 6 | 33950577 | C/G |
| 6 | 33967227 | G/A |
| 6 | 34011209 | A/G |
| 6 | 34024682 | C/T |
| 6 | 34056175 | T/C |
| 6 | 34086832 | C/T |
| 6 | 34121670 | G/A |
| 6 | 34137448 | A/C |
| 6 | 34163104 | G/A |
| 6 | 34172233 | G/A |
| 6 | 34202334 | G/A |
| 6 | 34212314 | G/A |
| 6 | 34253121 | G/A |
| 6 | 34254821 | A/G |
| 6 | 34283703 | G/A |
| 6 | 34287854 | A/G |
| 6 | 34339299 | A/G |
| 6 | 34356217 | T/C |
| 6 | 34428863 | C/G |
| 6 | 34431521 | G/A |
| 6 | 34440351 | A/T |
| 6 | 34487524 | C/G |
| 6 | 34514796 | G/C |
| 6 | 34524790 | T/G |
| 6 | 34530865 | T/C |
| 6 | 34569781 | C/T |
| 6 | 34575269 | G/A |
| 6 | 34590497 | A/G |
| 6 | 34607728 | A/G |
| 6 | 34639509 | C/G |
| 6 | 34641760 | T/C |
| 6 | 34697920 | C/T |
| 6 | 34727890 | A/G |
| 6 | 34752940 | G/A |
| 6 | 34760942 | A/T |
| 6 | 34775856 | A/T |
| 6 | 34907669 | C/A |
| 6 | 34908850 | G/T |
| 6 | 34920994 | C/G |
| 6 | 35181697 | A/C |
| 6 | 35200224 | G/A |
| 6 | 35226504 | C/G |
| 6 | 35234028 | C/T |
| 6 | 35285969 | C/G |
| 6 | 35306041 | T/C |
| 7 | 1650 | G/A |
| 7 | 22821 | C/T |
| 7 | 73783 | C/G |
| 7 | 81140 | A/C |
| 7 | 111284 | T/C |
| 7 | 116400 | A/G |
| 7 | 126743 | T/A |
| 7 | 155764 | C/T |
| 7 | 200456 | G/T |
| 7 | 204327 | T/C |
| 7 | 244212 | G/A |
| 7 | 281134 | A/T |
| 7 | 328218 | A/G |
| 7 | 372555 | C/T |
| 7 | 387728 | A/G |
| 7 | 401383 | G/T |
| 7 | 427623 | A/G |
| 7 | 448181 | C/T |
| 7 | 464747 | A/T |
| 7 | 464748 | T/G |
| 7 | 464749 | T/G |
| 7 | 464750 | T/G |
| 7 | 511894 | A/G |
| 7 | 528538 | C/G |
| 7 | 573122 | C/A |
| 7 | 639470 | A/G |
| 7 | 675188 | C/T |
| 7 | 682347 | G/A |
| 7 | 690175 | A/G |
| 7 | 721954 | T/C |
| 7 | 782039 | T/G |
| 7 | 785608 | G/A |
| 7 | 825822 | G/A |
| 7 | 853915 | G/A |
| 7 | 895234 | A/G |
| 7 | 897157 | C/T |
| 7 | 901269 | G/A |
| 7 | 911841 | A/G |
| 7 | 941655 | C/T |
| 7 | 972255 | T/C |
| 7 | 1002577 | G/T |
| 7 | 1011107 | T/G |
| 7 | 1023486 | C/T |
| 7 | 1054292 | T/C |
| 7 | 1079958 | A/T |
| 7 | 1090926 | A/G |
| 7 | 1119919 | G/A |
| 7 | 1142952 | T/C |
| 7 | 1173856 | C/G |
| 7 | 1204270 | T/C |
| 7 | 1239789 | C/T |
| 7 | 1256306 | T/C |
| 7 | 1261102 | G/A |
| 7 | 1304129 | C/T |
| 7 | 1322421 | T/C |
| 7 | 1326830 | C/T |
| 7 | 1360059 | G/T |
| 7 | 1393573 | C/T |
| 7 | 1415499 | T/G |
| 7 | 1459837 | A/G |
| 7 | 1559446 | T/A |
| 7 | 1576016 | G/A |
| 7 | 1595900 | A/G |
| 7 | 1613727 | G/A |
| 7 | 1625509 | G/C |
| 7 | 1632266 | G/A |
| 7 | 1651755 | G/A |
| 7 | 1678155 | A/G |
| 7 | 1680279 | G/A |
| 7 | 1685888 | T/C |
| 7 | 1734426 | G/A |
| 7 | 1736252 | A/G |
| 7 | 1764208 | C/A |
| 7 | 1784287 | A/C |
| 7 | 1806459 | G/A |
| 7 | 1807715 | C/G |
| 7 | 1831916 | G/C |
| 7 | 1842907 | C/T |
| 7 | 1879102 | T/C |
| 7 | 1909324 | T/C |
| 7 | 1935174 | G/C |
| 7 | 1944421 | C/A |
| 7 | 1965928 | A/G |
| 7 | 1984188 | G/A |
| 7 | 2017061 | T/C |
| 7 | 2029549 | T/G |
| 7 | 2069501 | T/C |
| 7 | 2087305 | G/C |
| 7 | 2094624 | A/T |
| 7 | 2106483 | A/C |
| 7 | 2126002 | C/T |
| 7 | 2146294 | G/A |
| 7 | 2158550 | G/T |
| 7 | 2187760 | G/A |
| 7 | 2200941 | A/T |
| 7 | 2226131 | T/C |
| 7 | 2234987 | T/C |
| 7 | 2264902 | C/T |
| 7 | 2287660 | C/A |
| 7 | 2290447 | T/A |
| 7 | 2323509 | A/G |
| 7 | 2333339 | G/A |
| 7 | 2352394 | A/T |
| 7 | 2370496 | T/C |
| 7 | 2372633 | T/A |
| 7 | 2411140 | C/T |
| 7 | 2421205 | C/T |
| 7 | 2448812 | A/G |
| 7 | 2451455 | T/C |
| 7 | 2466983 | A/T |
| 7 | 2475698 | C/T |
| 7 | 2494633 | T/A |
| 7 | 2504742 | G/T |
| 7 | 2521533 | G/A |
| 7 | 2553445 | C/T |
| 7 | 2572440 | G/A |
| 7 | 2591834 | T/C |
| 7 | 2600415 | C/T |
| 7 | 2637696 | T/A |
| 7 | 2654458 | G/A |
| 7 | 2676240 | G/C |
| 7 | 2706323 | T/G |
| 7 | 2733142 | T/C |
| 7 | 2738482 | C/T |
| 7 | 2775200 | T/A |
| 7 | 2809640 | A/G |
| 7 | 2817275 | C/T |
| 7 | 2832510 | A/C |
| 7 | 2865246 | T/C |
| 7 | 2879460 | G/A |
| 7 | 2895424 | A/T |
| 7 | 2901711 | G/A |
| 7 | 2922736 | T/C |
| 7 | 2925749 | G/T |
| 7 | 2953531 | G/A |
| 7 | 3005316 | G/A |
| 7 | 3031602 | T/C |
| 7 | 3034204 | C/T |
| 7 | 3088731 | T/G |
| 7 | 3114195 | T/C |
| 7 | 3130925 | C/T |
| 7 | 3139471 | C/T |
| 7 | 3162158 | C/T |
| 7 | 3175428 | T/C |
| 7 | 3184432 | A/C |
| 7 | 3185976 | G/A |
| 7 | 3210206 | T/G |
| 7 | 3227632 | A/T |
| 7 | 3262231 | G/A |
| 7 | 3308281 | C/G |
| 7 | 3315479 | C/T |
| 7 | 3357558 | T/C |
| 7 | 3360947 | C/A |
| 7 | 3388371 | G/A |
| 7 | 3412908 | C/T |
| 7 | 3416263 | C/A |
| 7 | 3441108 | C/T |
| 7 | 3464972 | T/C |
| 7 | 3483024 | C/G |
| 7 | 3494812 | C/T |
| 7 | 3518560 | G/A |
| 7 | 3538974 | A/C |
| 7 | 3546848 | C/T |
| 7 | 3568676 | G/A |
| 7 | 3583817 | G/T |
| 7 | 3611945 | A/G |
| 7 | 3617119 | G/C |
| 7 | 3653258 | G/A |
| 7 | 3662011 | C/T |
| 7 | 3663687 | C/A |
| 7 | 3696742 | C/T |
| 7 | 3736473 | T/A |
| 7 | 3791162 | A/G |
| 7 | 3814651 | C/T |
| 7 | 3838920 | G/A |
| 7 | 3866469 | T/A |
| 7 | 3868424 | A/G |
| 7 | 3898044 | C/T |
| 7 | 3899450 | A/G |
| 7 | 3902048 | C/G |
| 7 | 3911948 | A/G |
| 7 | 3930401 | G/A |
| 7 | 3931801 | T/C |
| 7 | 3963106 | A/C |
| 7 | 3998812 | A/G |
| 7 | 4015112 | A/T |
| 7 | 4034634 | C/T |
| 7 | 4051164 | T/G |
| 7 | 4053074 | A/G |
| 7 | 4090378 | C/A |
| 7 | 4106448 | A/G |
| 7 | 4129255 | T/C |
| 7 | 4141796 | A/G |
| 7 | 4147756 | C/T |
| 7 | 4173712 | A/G |
| 7 | 4200391 | C/A |
| 7 | 4209239 | A/G |
| 7 | 4256876 | G/A |
| 7 | 4263792 | C/T |
| 7 | 4293575 | C/T |
| 7 | 4320681 | T/C |
| 7 | 4346445 | C/G |
| 7 | 4352549 | G/T |
| 7 | 4362718 | C/G |
| 7 | 4427873 | T/C |
| 7 | 4457114 | T/C |
| 7 | 4473670 | C/T |
| 7 | 4515684 | G/A |
| 7 | 4527672 | C/T |
| 7 | 4531870 | T/C |
| 7 | 4558074 | C/T |
| 7 | 4583029 | G/A |
| 7 | 4627206 | G/A |
| 7 | 4650941 | A/G |
| 7 | 4658730 | C/T |
| 7 | 4682690 | A/G |
| 7 | 4696569 | G/A |
| 7 | 4714385 | A/G |
| 7 | 4751950 | A/T |
| 7 | 4783844 | A/G |
| 7 | 4798580 | T/C |
| 7 | 4824513 | G/A |
| 7 | 4854770 | T/G |
| 7 | 4857115 | G/A |
| 7 | 4875097 | G/A |
| 7 | 4876991 | C/T |
| 7 | 4894314 | G/T |
| 7 | 4925934 | C/T |
| 7 | 4929890 | A/G |
| 7 | 4970796 | G/A |
| 7 | 4979665 | G/A |
| 7 | 4992134 | C/T |
| 7 | 5005831 | A/G |
| 7 | 5033764 | G/A |
| 7 | 5037498 | C/T |
| 7 | 5068637 | A/G |
| 7 | 5070233 | G/A |
| 7 | 5106852 | C/T |
| 7 | 5117360 | C/T |
| 7 | 5146880 | C/G |
| 7 | 5171715 | G/T |
| 7 | 5187515 | G/A |
| 7 | 5200560 | A/G |
| 7 | 5202199 | T/C |
| 7 | 5238548 | C/T |
| 7 | 5240373 | G/C |
| 7 | 5278167 | A/G |
| 7 | 5286880 | G/A |
| 7 | 5334355 | T/C |
| 7 | 5347837 | G/A |
| 7 | 5362114 | C/T |
| 7 | 5382114 | T/C |
| 7 | 5383236 | C/T |
| 7 | 5413382 | G/A |
| 7 | 5431883 | A/T |
| 7 | 5445099 | C/T |
| 7 | 5481639 | G/A |
| 7 | 5504264 | A/G |
| 7 | 5506355 | T/C |
| 7 | 5536058 | G/A |
| 7 | 5549420 | A/G |
| 7 | 5571835 | A/C |
| 7 | 5579247 | G/C |
| 7 | 5580596 | A/T |
| 7 | 5597939 | C/T |
| 7 | 5631795 | G/A |
| 7 | 5634045 | T/C |
| 7 | 5653264 | A/T |
| 7 | 5674264 | G/A |
| 7 | 5687486 | G/A |
| 7 | 5713773 | C/T |
| 7 | 5719879 | C/G |
| 7 | 5735262 | G/A |
| 7 | 5774051 | T/A |
| 7 | 5798912 | T/A |
| 7 | 5800091 | C/T |
| 7 | 5821662 | T/A |
| 7 | 5830438 | A/T |
| 7 | 5875758 | T/G |
| 7 | 5880321 | G/A |
| 7 | 5911680 | G/C |
| 7 | 5913861 | C/A |
| 7 | 5947836 | C/T |
| 7 | 5958054 | T/A |
| 7 | 5998060 | G/C |
| 7 | 6002672 | T/C |
| 7 | 6014712 | A/C |
| 7 | 6048339 | T/C |
| 7 | 6077129 | C/A |
| 7 | 6098006 | C/T |
| 7 | 6137838 | A/C |
| 7 | 6147572 | G/A |
| 7 | 6160037 | G/T |
| 7 | 6166795 | C/G |
| 7 | 6188134 | C/A |
| 7 | 5230965 | G/T |
| 7 | 6236799 | T/C |
| 7 | 6250717 | G/A |
| 7 | 6264330 | C/T |
| 7 | 6271874 | G/A |
| 7 | 6274690 | C/G |
| 7 | 6340279 | A/T |
| 7 | 6344064 | C/T |
| 7 | 6366778 | T/C |
| 7 | 6367821 | A/G |
| 7 | 6396406 | C/G |
| 7 | 6422827 | C/T |
| 7 | 6445164 | C/T |
| 7 | 6469118 | G/A |
| 7 | 6499844 | C/T |
| 7 | 6532998 | T/A |
| 7 | 6556424 | A/C |
| 7 | 6565520 | A/G |
| 7 | 6584400 | G/A |
| 7 | 6616097 | C/T |
| 7 | 6631207 | G/A |
| 7 | 6689928 | C/T |
| 7 | 6694910 | C/T |
| 7 | 6696016 | C/T |
| 7 | 6724422 | G/A |
| 7 | 6766389 | C/T |
| 7 | 6773956 | C/T |
| 7 | 6786293 | G/A |
| 7 | 6811400 | G/A |
| 7 | 6835834 | C/T |
| 7 | 6843668 | A/G |
| 7 | 6894371 | A/T |
| 7 | 6920495 | G/A |
| 7 | 6927433 | T/C |
| 7 | 6935364 | G/T |
| 7 | 6948919 | C/T |
| 7 | 6972443 | T/C |
| 7 | 6995914 | G/A |
| 7 | 7028359 | G/A |
| 7 | 7031784 | G/A |
| 7 | 7065683 | G/A |
| 7 | 7088577 | C/T |
| 7 | 7112296 | G/A |
| 7 | 7133927 | T/C |
| 7 | 7171590 | A/C |
| 7 | 7173621 | A/G |
| 7 | 7214318 | A/G |
| 7 | 7215530 | T/G |
| 7 | 7262995 | C/T |
| 7 | 7269647 | A/G |
| 7 | 7312559 | G/A |
| 7 | 7325924 | G/A |
| 7 | 7359768 | T/C |
| 7 | 7380346 | T/C |
| 7 | 7422836 | T/G |
| 7 | 7467958 | C/G |
| 7 | 7471261 | T/C |
| 7 | 7472362 | C/T |
| 7 | 7511688 | C/G |
| 7 | 7552569 | A/G |
| 7 | 7563136 | G/A |
| 7 | 7577275 | C/T |
| 7 | 7605717 | G/A |
| 7 | 7633965 | A/G |
| 7 | 7683088 | A/G |
| 7 | 7714681 | G/A |
| 7 | 7751498 | C/A |
| 7 | 7782074 | T/C |
| 7 | 7791245 | T/C |
| 7 | 7836906 | C/A |
| 7 | 7876760 | G/A |
| 7 | 7889674 | C/T |
| 7 | 7892822 | C/T |
| 7 | 7927299 | G/T |
| 7 | 7944029 | G/A |
| 7 | 7958220 | G/A |
| 7 | 7962477 | C/T |
| 7 | 7984187 | C/T |
| 7 | 8007294 | T/A |
| 7 | 8021470 | G/T |
| 7 | 8034243 | A/C |
| 7 | 8045667 | C/T |
| 7 | 8094911 | C/A |
| 7 | 8108960 | C/T |
| 7 | 8115209 | G/A |
| 7 | 8137463 | C/T |
| 7 | 8147128 | G/A |
| 7 | 8173107 | G/A |
| 7 | 8180586 | C/A |
| 7 | 8205415 | G/A |
| 7 | 8208314 | C/T |
| 7 | 8242667 | T/C |
| 7 | 8249370 | A/T |
| 7 | 8270543 | C/T |
| 7 | 8281045 | G/A |
| 7 | 8308594 | C/A |
| 7 | 8339098 | A/G |
| 7 | 8353929 | G/A |
| 7 | 8363336 | A/G |
| 7 | 8387441 | C/A |
| 7 | 8388627 | A/G |
| 7 | 8413988 | A/C |
| 7 | 8426405 | A/G |
| 7 | 8437561 | C/A |
| 7 | 8458871 | A/G |
| 7 | 8473398 | T/G |
| 7 | 8477079 | T/C |
| 7 | 8501215 | A/G |
| 7 | 8510853 | A/G |
| 7 | 8529161 | C/G |
| 7 | 8544961 | A/G |
| 7 | 8558154 | C/G |
| 7 | 8599983 | A/G |
| 7 | 8611796 | C/A |
| 7 | 8634050 | G/A |
| 7 | 8640199 | A/G |
| 7 | 8661828 | G/T |
| 7 | 8673088 | C/T |
| 7 | 8686486 | C/G |
| 7 | 8724373 | C/T |
| 7 | 8725575 | T/A |
| 7 | 8755449 | T/C |
| 7 | 8771375 | A/C |
| 7 | 8776549 | C/T |
| 7 | 8808483 | C/A |
| 7 | 8834530 | T/A |
| 7 | 8837598 | T/C |
| 7 | 8911118 | T/A |
| 7 | 8950499 | G/A |
| 7 | 8990348 | C/T |
| 7 | 8994870 | T/C |
| 7 | 9015328 | G/A |
| 7 | 9026806 | G/A |
| 7 | 9079768 | A/G |
| 7 | 9100921 | T/G |
| 7 | 9112016 | G/C |
| 7 | 9135719 | T/G |
| 7 | 9155349 | T/C |
| 7 | 9162762 | C/T |
| 7 | 9185161 | C/T |
| 7 | 9191732 | C/T |
| 7 | 9221923 | G/A |
| 7 | 9229394 | C/T |
| 7 | 9250757 | G/A |
| 7 | 9271292 | C/T |
| 7 | 9279109 | A/G |
| 7 | 9331085 | T/C |
| 7 | 9339674 | C/T |
| 7 | 9381928 | G/A |
| 7 | 9401621 | G/A |
| 7 | 9404459 | G/A |
| 7 | 9422459 | A/G |
| 7 | 9442461 | G/A |
| 7 | 9470875 | C/T |
| 7 | 9534076 | G/A |
| 7 | 9540666 | T/C |
| 7 | 9559246 | G/A |
| 7 | 9581561 | A/G |
| 7 | 9609030 | T/A |
| 7 | 9643151 | C/T |
| 7 | 9652073 | C/T |
| 7 | 9663421 | C/G |
| 7 | 9709707 | G/T |
| 7 | 9721197 | A/T |
| 7 | 9760191 | G/A |
| 7 | 9773031 | T/G |
| 7 | 9806479 | G/A |
| 7 | 9814852 | C/T |
| 7 | 9849467 | T/A |
| 7 | 9853049 | G/A |
| 7 | 9881458 | T/C |
| 7 | 9882839 | T/C |
| 7 | 9905746 | T/C |
| 7 | 9929413 | G/A |
| 7 | 9931740 | G/A |
| 7 | 9935152 | T/C |
| 7 | 9963062 | A/G |
| 7 | 9985442 | C/G |
| 7 | 9998404 | G/T |
| 7 | 10035456 | G/A |
| 7 | 10057328 | C/T |
| 7 | 10092983 | G/A |
| 7 | 10094013 | G/C |
| 7 | 10118709 | T/C |
| 7 | 10120945 | C/T |
| 7 | 10169887 | A/G |
| 7 | 10177688 | A/G |
| 7 | 10182356 | A/G |
| 7 | 10206433 | T/C |
| 7 | 10238514 | G/C |
| 7 | 10261308 | A/G |
| 7 | 10277017 | G/T |
| 7 | 10293666 | C/A |
| 7 | 10326211 | T/A |
| 7 | 10355095 | C/T |
| 7 | 10375015 | A/T |
| 7 | 10385521 | C/T |
| 7 | 10407527 | C/T |
| 7 | 10410773 | G/A |
| 7 | 10427806 | C/T |
| 7 | 10462404 | T/C |
| 7 | 10489735 | C/G |
| 7 | 10502207 | A/T |
| 7 | 10550445 | G/A |
| 7 | 10570790 | G/A |
| 7 | 10592554 | C/T |
| 7 | 10599857 | A/G |
| 7 | 10639614 | T/C |
| 7 | 10642497 | C/T |
| 7 | 10672265 | T/C |
| 7 | 10679422 | A/G |
| 7 | 10684037 | C/T |
| 7 | 10690003 | G/A |
| 7 | 10728977 | G/A |
| 7 | 10732441 | A/G |
| 7 | 10742750 | T/C |
| 7 | 10777482 | T/A |
| 7 | 10796481 | T/C |
| 7 | 10819499 | C/T |
| 7 | 10822614 | G/C |
| 7 | 10854878 | A/C |
| 7 | 10879036 | C/T |
| 7 | 10881829 | C/T |
| 7 | 10893184 | G/C |
| 7 | 10899806 | A/G |
| 7 | 10926531 | C/G |
| 7 | 10964666 | T/G |
| 7 | 10984182 | C/T |
| 7 | 11001197 | A/C |
| 7 | 11010836 | C/T |
| 7 | 11015078 | T/C |
| 7 | 11047101 | C/T |
| 7 | 11056786 | G/T |
| 7 | 11072052 | C/G |
| 7 | 11107224 | C/A |
| 7 | 11155324 | C/T |
| 7 | 11156333 | T/C |
| 7 | 11174651 | C/A |
| 7 | 11207515 | G/A |
| 7 | 11209470 | T/C |
| 7 | 11226341 | C/T |
| 7 | 11228393 | C/T |
| 7 | 11251814 | A/T |
| 7 | 11269796 | A/C |
| 7 | 11289884 | C/A |
| 7 | 11310617 | T/C |
| 7 | 11342249 | C/T |
| 7 | 11369321 | G/A |
| 7 | 11370728 | A/C |
| 7 | 11384669 | G/C |
| 7 | 11412973 | C/A |
| 7 | 11454098 | A/C |
| 7 | 11473085 | C/T |
| 7 | 11499626 | A/G |
| 7 | 11520967 | A/C |
| 7 | 11551560 | T/C |
| 7 | 11580815 | T/C |
| 7 | 11604351 | G/C |
| 7 | 11621071 | G/A |
| 7 | 11650937 | C/T |
| 7 | 11676523 | T/G |
| 7 | 11695808 | C/T |
| 7 | 11716533 | G/A |
| 7 | 11737726 | G/A |
| 7 | 11738883 | T/G |
| 7 | 11819285 | C/T |
| 7 | 11832601 | G/A |
| 7 | 11863985 | A/C |
| 7 | 11905813 | G/A |
| 7 | 11912335 | G/A |
| 7 | 11946998 | G/A |
| 7 | 11958065 | G/A |
| 7 | 11995709 | T/C |
| 7 | 12006485 | T/C |
| 7 | 12030028 | A/G |
| 7 | 12059323 | G/A |
| 7 | 12069792 | T/C |
| 7 | 12094173 | C/A |
| 7 | 12102923 | C/A |
| 7 | 12132026 | A/T |
| 7 | 12171915 | T/A |
| 7 | 12194335 | T/C |
| 7 | 12209436 | A/G |
| 7 | 12210760 | C/T |
| 7 | 12259736 | G/A |
| 7 | 12271034 | G/A |
| 7 | 12282882 | G/T |
| 7 | 12303889 | T/C |
| 7 | 12323897 | T/A |
| 7 | 12349654 | C/G |
| 7 | 12370732 | T/G |
| 7 | 12372259 | G/C |
| 7 | 12413400 | A/C |
| 7 | 12416824 | T/C |
| 7 | 12431156 | C/G |
| 7 | 12457712 | T/C |
| 7 | 12471899 | G/A |
| 7 | 12503954 | T/G |
| 7 | 12506630 | C/T |
| 7 | 12530190 | C/T |
| 7 | 12539985 | G/A |
| 7 | 12558405 | A/T |
| 7 | 12577019 | T/C |
| 7 | 12578662 | A/G |
| 7 | 12615006 | C/T |
| 7 | 12619422 | G/A |
| 7 | 12653421 | T/C |
| 7 | 12660513 | C/T |
| 7 | 12662549 | G/A |
| 7 | 12695131 | G/C |
| 7 | 12736785 | G/A |
| 7 | 12745332 | A/G |
| 7 | 12751333 | G/A |
| 7 | 12767161 | G/A |
| 7 | 12794153 | C/G |
| 7 | 12807845 | T/C |
| 7 | 12814846 | G/T |
| 7 | 12823939 | T/C |
| 7 | 12845248 | C/T |
| 7 | 12864146 | T/C |
| 7 | 12887977 | A/G |
| 7 | 12892978 | T/C |
| 7 | 12923618 | T/C |
| 7 | 12951652 | T/G |
| 7 | 12962347 | C/T |
| 7 | 12993482 | G/A |
| 7 | 13008683 | T/C |
| 7 | 13024960 | T/A |
| 7 | 13026218 | A/C |
| 7 | 13055739 | A/T |
| 7 | 13083534 | G/A |
| 7 | 13116774 | G/A |
| 7 | 13124354 | C/T |
| 7 | 13142466 | G/A |
| 7 | 13143681 | A/G |
| 7 | 13186053 | A/G |
| 7 | 13201508 | T/A |
| 7 | 13205121 | A/G |
| 7 | 13312230 | T/C |
| 7 | 13314215 | A/G |
| 7 | 13321642 | C/T |
| 7 | 13344179 | A/G |
| 7 | 13364705 | A/C |
| 7 | 13385224 | A/G |
| 7 | 13408742 | G/A |
| 7 | 13420885 | T/C |
| 7 | 13448395 | C/T |
| 7 | 13472818 | T/C |
| 7 | 13488990 | A/G |
| 7 | 13559821 | A/G |
| 7 | 13568865 | G/A |
| 7 | 13572777 | A/G |
| 7 | 13590972 | C/G |
| 7 | 13608040 | G/A |
| 7 | 13643283 | A/T |
| 7 | 13654258 | T/C |
| 7 | 13691716 | C/A |
| 7 | 13710481 | G/C |
| 7 | 13766828 | A/G |
| 7 | 13772390 | A/G |
| 7 | 13780551 | A/G |
| 7 | 13827800 | C/A |
| 7 | 13835900 | G/A |
| 7 | 13847134 | A/T |
| 7 | 13865869 | G/A |
| 7 | 13870081 | G/A |
| 7 | 13906451 | A/G |
| 7 | 13907767 | G/A |
| 7 | 13932721 | C/A |
| 7 | 13936903 | T/C |
| 7 | 13952536 | G/A |
| 7 | 13960174 | C/T |
| 7 | 14000765 | G/A |
| 7 | 14007387 | T/C |
| 7 | 14029253 | T/C |
| 7 | 14048089 | C/T |
| 7 | 14059988 | A/G |
| 7 | 14122468 | G/A |
| 7 | 14130711 | C/T |
| 7 | 14180095 | C/T |
| 7 | 14183584 | T/G |
| 7 | 14190091 | T/C |
| 7 | 14192131 | G/A |
| 7 | 14232127 | C/G |
| 7 | 14248343 | A/G |
| 7 | 14264658 | A/G |
| 7 | 14267798 | C/T |
| 7 | 14300430 | C/T |
| 7 | 14319618 | T/A |
| 7 | 14334388 | T/C |
| 7 | 14360341 | C/T |
| 7 | 14381008 | C/T |
| 7 | 14397542 | C/T |
| 7 | 14402092 | C/T |
| 7 | 14423264 | C/T |
| 7 | 14437040 | T/G |
| 7 | 14460594 | C/G |
| 7 | 14495257 | G/A |
| 7 | 14546917 | A/G |
| 7 | 14556280 | G/A |
| 7 | 14567108 | T/C |
| 7 | 14587028 | T/A |
| 7 | 14592126 | G/A |
| 7 | 14610791 | G/C |
| 7 | 14618655 | G/C |
| 7 | 14657697 | G/A |
| 7 | 14659678 | T/C |
| 7 | 14678778 | T/C |
| 7 | 14684349 | C/A |
| 7 | 14712557 | G/T |
| 7 | 14715808 | G/A |
| 7 | 14735205 | A/C |
| 7 | 14755789 | A/G |
| 7 | 14784395 | G/A |
| 7 | 14808618 | G/T |
| 7 | 14812697 | G/A |
| 7 | 14828865 | A/G |
| 7 | 14854790 | A/G |
| 7 | 14868773 | C/T |
| 7 | 14890360 | A/G |
| 7 | 14935091 | G/A |
| 7 | 14954861 | C/T |
| 7 | 14999205 | C/T |
| 7 | 15009776 | T/C |
| 7 | 15027055 | T/C |
| 7 | 15032027 | G/A |
| 7 | 15078563 | T/C |
| 7 | 15092708 | A/G |
| 7 | 15121305 | G/A |
| 7 | 15133092 | A/T |
| 7 | 15150147 | G/T |
| 7 | 15187087 | T/C |
| 7 | 15222573 | A/G |
| 7 | 15244641 | C/T |
| 7 | 15280439 | G/A |
| 7 | 15281588 | C/A |
| 7 | 15314626 | C/A |
| 7 | 15329849 | A/C |
| 7 | 15350902 | T/C |
| 7 | 15375721 | A/G |
| 7 | 15394265 | G/A |
| 7 | 15416257 | C/T |
| 7 | 15424541 | C/T |
| 7 | 15432872 | C/T |
| 7 | 15464642 | G/A |
| 7 | 15473814 | C/T |
| 7 | 15486287 | A/G |
| 7 | 15499452 | C/T |
| 7 | 15524184 | T/C |
| 7 | 15537823 | T/C |
| 7 | 15548900 | G/C |
| 7 | 15550180 | A/T |
| 7 | 15573925 | G/A |
| 7 | 15586429 | A/G |
| 7 | 15611103 | G/A |
| 7 | 15621928 | C/A |
| 7 | 15651058 | A/G |
| 7 | 15659739 | A/G |
| 7 | 15662037 | G/C |
| 7 | 15693163 | A/T |
| 7 | 15706916 | T/G |
| 7 | 15722089 | C/A |
| 7 | 15723210 | G/T |
| 7 | 15770430 | T/C |
| 7 | 15782511 | A/C |
| 7 | 15791099 | A/T |
| 7 | 15836179 | T/C |
| 7 | 15841486 | G/A |
| 7 | 15855761 | T/A |
| 7 | 15889518 | T/C |
| 7 | 15896465 | C/T |
| 7 | 15905715 | G/A |
| 7 | 15908821 | C/T |
| 7 | 15940784 | A/G |
| 7 | 15972773 | T/C |
| 7 | 15988267 | C/G |
| 7 | 16012998 | C/G |
| 7 | 16023960 | C/A |
| 7 | 16058016 | T/G |
| 7 | 16078733 | G/C |
| 7 | 16081182 | T/C |
| 7 | 16083199 | G/T |
| 7 | 16128953 | A/C |
| 7 | 16134835 | A/G |
| 7 | 16157375 | G/C |
| 7 | 16164435 | C/A |
| 7 | 16170281 | A/G |
| 7 | 16220212 | T/C |
| 7 | 16223320 | C/A |
| 7 | 16245904 | C/T |
| 7 | 16250497 | G/C |
| 7 | 16268696 | G/T |
| 7 | 16299703 | T/A |
| 7 | 16301158 | A/G |
| 7 | 16332985 | G/A |
| 7 | 16350478 | A/G |
| 7 | 16385085 | T/A |
| 7 | 16438886 | G/A |
| 7 | 16441628 | C/T |
| 7 | 16444513 | G/A |
| 7 | 16492923 | C/A |
| 7 | 16503129 | A/G |
| 7 | 16538851 | G/A |
| 7 | 16559902 | A/G |
| 7 | 16581378 | A/G |
| 7 | 16583085 | C/G |
| 7 | 16592069 | G/A |
| 7 | 16601479 | T/C |
| 7 | 16642420 | C/T |
| 7 | 16662741 | T/C |
| 7 | 16688959 | C/T |
| 7 | 16735789 | G/A |
| 7 | 16739562 | C/T |
| 7 | 16763536 | A/C |
| 7 | 16769072 | T/A |
| 7 | 16792103 | C/T |
| 7 | 16824896 | C/T |
| 7 | 16828084 | G/A |
| 7 | 16842930 | G/C |
| 7 | 16873705 | G/A |
| 7 | 16908663 | C/T |
| 7 | 16911041 | C/T |
| 7 | 16936780 | G/A |
| 7 | 16970107 | G/A |
| 7 | 17000458 | G/A |
| 7 | 17003195 | G/A |
| 7 | 17033492 | G/A |
| 7 | 17039831 | G/T |
| 7 | 17062771 | G/A |
| 7 | 17080080 | C/A |
| 7 | 17081976 | G/A |
| 7 | 17122653 | G/A |
| 7 | 17131657 | G/A |
| 7 | 17153519 | G/A |
| 7 | 17168732 | A/G |
| 7 | 17197083 | G/A |
| 7 | 17198325 | G/C |
| 7 | 17283800 | C/A |
| 7 | 17291793 | C/G |
| 7 | 17328319 | G/A |
| 7 | 17398949 | G/A |
| 7 | 17406765 | C/G |
| 7 | 17427210 | T/G |
| 7 | 17452068 | G/A |
| 7 | 17454306 | C/T |
| 7 | 17460123 | G/A |
| 7 | 17504212 | T/C |
| 7 | 17566310 | C/A |
| 7 | 17800267 | T/C |
| 7 | 17825664 | G/A |
| 7 | 17902586 | G/A |
| 7 | 17910245 | T/C |
| 7 | 17935813 | G/T |
| 7 | 17952255 | G/A |
| 7 | 18016255 | C/T |
| 7 | 18046113 | A/G |
| 7 | 18077881 | G/A |
| 7 | 18115547 | A/G |
| 7 | 18125952 | G/C |
| 7 | 18161057 | T/C |
| 7 | 18261673 | A/G |
| 7 | 18270730 | G/A |
| 7 | 18313774 | A/G |
| 7 | 18339652 | C/T |
| 7 | 18365751 | T/A |
| 7 | 18436985 | C/T |
| 7 | 18461017 | T/C |
| 7 | 18497130 | C/T |
| 7 | 18509254 | G/A |
| 7 | 18535281 | C/T |
| 7 | 18568268 | G/A |
| 7 | 18571033 | C/T |
| 7 | 18626800 | A/G |
| 7 | 18630785 | C/T |
| 7 | 18662890 | G/A |
| 7 | 18713655 | T/C |
| 7 | 18734900 | C/T |
| 7 | 18788004 | G/A |
| 7 | 18801921 | C/T |
| 7 | 18832875 | G/A |
| 7 | 18851398 | G/A |
| 7 | 18970710 | C/T |
| 7 | 18974000 | G/T |
| 7 | 19039171 | G/A |
| 7 | 19040466 | C/T |
| 7 | 19075719 | G/A |
| 7 | 19077250 | C/T |
| 7 | 19088619 | G/A |
| 7 | 19090231 | G/A |
| 7 | 19111867 | T/C |
| 7 | 19115992 | A/G |
| 7 | 19140546 | T/C |
| 7 | 19154468 | A/G |
| 7 | 19174219 | C/T |
| 7 | 19190493 | C/A |
| 7 | 19217573 | A/C |
| 7 | 19232408 | A/C |
| 7 | 19255332 | G/A |
| 7 | 19269621 | C/T |
| 7 | 19274910 | G/A |
| 7 | 19290509 | G/A |
| 7 | 19319103 | C/G |
| 7 | 19353726 | G/A |
| 7 | 19398988 | A/G |
| 7 | 19403303 | G/A |
| 7 | 19416905 | T/C |
| 7 | 19431485 | G/A |
| 7 | 19441506 | G/A |
| 7 | 19443823 | T/A |
| 7 | 19476768 | C/T |
| 7 | 19478099 | G/T |
| 7 | 19500785 | C/G |
| 7 | 19508421 | C/A |
| 7 | 19540953 | T/C |
| 7 | 19542080 | C/G |
| 7 | 19583299 | C/T |
| 7 | 19591405 | C/T |
| 7 | 19596678 | C/T |
| 7 | 19643297 | G/A |
| 7 | 19657961 | C/T |
| 7 | 19685959 | C/A |
| 7 | 19696696 | A/G |
| 7 | 19719927 | T/C |
| 7 | 19742806 | C/G |
| 7 | 19748900 | G/A |
| 7 | 19770026 | C/T |
| 7 | 19797653 | G/A |
| 7 | 19805006 | G/T |
| 7 | 19833561 | G/T |
| 7 | 19848523 | G/A |
| 7 | 19865675 | G/T |
| 7 | 19877209 | A/C |
| 7 | 19895246 | A/T |
| 7 | 19909638 | T/C |
| 7 | 19933170 | C/T |
| 7 | 19948068 | G/A |
| 7 | 19960245 | A/C |
| 7 | 19964268 | T/C |
| 7 | 19999435 | A/G |
| 7 | 20009823 | G/A |
| 7 | 20021887 | T/C |
| 7 | 20065004 | A/T |
| 7 | 20083710 | C/G |
| 7 | 20119043 | G/A |
| 7 | 20157168 | C/T |
| 7 | 20159304 | C/T |
| 7 | 20177769 | G/T |
| 7 | 20180482 | A/G |
| 7 | 20194640 | C/G |
| 7 | 20201705 | C/T |
| 7 | 20220881 | C/T |
| 7 | 20257629 | G/T |
| 7 | 20259851 | A/G |
| 7 | 20288697 | A/T |
| 7 | 20304288 | C/T |
| 7 | 20337860 | G/A |
| 7 | 20339862 | T/C |
| 7 | 20350514 | C/T |
| 7 | 20376632 | A/T |
| 7 | 20448395 | A/G |
| 7 | 20481227 | T/C |
| 7 | 20503512 | G/T |
| 7 | 20522683 | C/A |
| 7 | 20560610 | G/A |
| 7 | 20563812 | G/A |
| 7 | 20599559 | A/G |
| 7 | 20602545 | G/T |
| 7 | 20610252 | A/G |
| 7 | 20642857 | T/C |
| 7 | 20688664 | G/A |
| 7 | 20691775 | A/C |
| 7 | 20705734 | C/A |
| 7 | 20910602 | C/A |
| 7 | 20759107 | G/A |
| 7 | 20760555 | G/C |
| 7 | 20791612 | T/C |
| 7 | 20823082 | G/C |
| 7 | 20852810 | A/G |
| 7 | 20886501 | T/C |
| 7 | 20899902 | C/T |
| 7 | 20924764 | A/G |
| 7 | 20928934 | A/G |
| 7 | 20961329 | C/T |
| 7 | 20974376 | C/A |
| 7 | 20990715 | T/A |
| 7 | 21013197 | C/T |
| 7 | 21034131 | C/T |
| 7 | 21038941 | C/T |
| 7 | 21079588 | G/A |
| 7 | 21102425 | A/G |
| 7 | 21105567 | G/C |
| 7 | 21120416 | G/A |
| 7 | 21143048 | G/A |
| 7 | 21153426 | T/G |
| 7 | 21155331 | T/C |
| 7 | 21209607 | G/A |
| 7 | 21213347 | G/A |
| 7 | 21254922 | T/A |
| 7 | 21260521 | G/A |
| 7 | 21274316 | A/G |
| 7 | 21332521 | T/C |
| 7 | 21390643 | T/C |
| 7 | 21429911 | C/T |
| 7 | 21443400 | T/G |
| 7 | 21478224 | C/G |
| 7 | 21498677 | G/A |
| 7 | 21506388 | G/A |
| 7 | 21518510 | T/A |
| 7 | 21522161 | A/T |
| 7 | 21550270 | A/G |
| 7 | 21579255 | G/A |
| 7 | 21587135 | C/T |
| 7 | 21619428 | G/A |
| 7 | 21623975 | C/T |
| 7 | 21630823 | G/A |
| 7 | 21645242 | A/G |
| 7 | 21662958 | A/T |
| 7 | 21689954 | C/G |
| 7 | 21695102 | C/T |
| 7 | 21735237 | T/C |
| 7 | 21745271 | G/T |
| 7 | 21775054 | C/T |
| 7 | 21778554 | G/C |
| 7 | 21780717 | G/A |
| 7 | 21812697 | C/T |
| 7 | 21840937 | A/G |
| 7 | 21874930 | G/A |
| 7 | 21900454 | T/C |
| 7 | 21907965 | T/C |
| 7 | 21950844 | C/G |
| 7 | 21952370 | T/C |
| 7 | 21977919 | T/G |
| 7 | 21982975 | C/A |
| 7 | 21999727 | C/T |
| 7 | 22032240 | C/T |
| 7 | 22033975 | A/G |
| 7 | 22057141 | A/G |
| 7 | 22101045 | C/T |
| 7 | 22106086 | C/G |
| 7 | 22133917 | C/T |
| 7 | 22141914 | C/T |
| 7 | 22148628 | C/T |
| 7 | 22182201 | A/G |
| 7 | 22208739 | T/C |
| 7 | 22243769 | C/T |
| 7 | 22252066 | G/A |
| 7 | 22277974 | G/A |
| 7 | 22284657 | G/T |
| 7 | 22305756 | C/T |
| 7 | 22308527 | C/T |
| 7 | 22336483 | C/G |
| 7 | 22337648 | G/C |
| 7 | 22372236 | T/C |
| 7 | 22384816 | T/C |
| 7 | 22414099 | A/G |
| 7 | 22428966 | C/T |
| 7 | 22495330 | T/C |
| 7 | 22496576 | C/A |
| 7 | 22504587 | C/T |
| 7 | 22524876 | C/T |
| 7 | 22550983 | G/T |
| 7 | 22553805 | C/T |
| 7 | 22577225 | G/A |
| 7 | 22579443 | G/A |
| 7 | 22613326 | A/T |
| 7 | 22810575 | G/A |
| 7 | 22874289 | G/A |
| 7 | 22877797 | T/C |
| 7 | 22919935 | A/G |
| 7 | 22921790 | C/A |
| 7 | 22941901 | A/G |
| 7 | 22958484 | G/T |
| 7 | 22999680 | C/T |
| 7 | 23004805 | G/T |
| 7 | 23010368 | T/G |
| 7 | 23023320 | A/G |
| 7 | 23055225 | G/A |
| 7 | 23105677 | C/T |
| 7 | 23111653 | C/A |
| 7 | 23141001 | C/G |
| 7 | 23159778 | G/A |
| 7 | 23163497 | G/A |
| 7 | 23187057 | T/C |
| 7 | 23196994 | C/T |
| 7 | 23225658 | C/G |
| 7 | 23298078 | C/T |
| 7 | 23311767 | C/T |
| 7 | 23314328 | G/A |
| 7 | 23349204 | A/G |
| 7 | 23389144 | C/G |
| 7 | 23433198 | G/A |
| 7 | 23449023 | T/C |
| 7 | 23488045 | C/A |
| 7 | 23494796 | T/C |
| 7 | 23500579 | T/C |
| 7 | 23526352 | A/G |
| 7 | 23557163 | G/A |
| 7 | 23650785 | C/G |
| 7 | 23673385 | C/T |
| 7 | 23676540 | C/T |
| 7 | 23710465 | C/T |
| 7 | 23722620 | C/T |
| 7 | 23740494 | A/T |
| 7 | 23750510 | G/A |
| 7 | 23774694 | G/A |
| 7 | 23811715 | A/C |
| 7 | 23824009 | G/T |
| 7 | 23843924 | C/A |
| 7 | 23854808 | G/A |
| 7 | 23875594 | G/A |
| 7 | 23883655 | T/A |
| 7 | 23921097 | T/C |
| 7 | 23942369 | C/T |
| 7 | 23962085 | A/T |
| 7 | 23973322 | C/G |
| 7 | 24003787 | A/C |
| 7 | 24040135 | G/A |
| 7 | 24060146 | C/T |
| 7 | 24081873 | G/C |
| 7 | 24102400 | T/A |
| 7 | 24107681 | T/A |
| 7 | 24134477 | T/C |
| 7 | 24138429 | T/G |
| 7 | 24150761 | A/G |
| 7 | 24157436 | G/C |
| 7 | 24204035 | A/C |
| 7 | 24208354 | G/A |
| 7 | 24227449 | A/G |
| 7 | 24228567 | T/C |
| 7 | 24264963 | A/G |
| 7 | 24273730 | G/A |
| 7 | 24278404 | A/G |
| 7 | 24321025 | C/T |
| 7 | 24345484 | G/A |
| 7 | 24352503 | G/C |
| 7 | 24385047 | T/C |
| 7 | 24403308 | T/C |
| 7 | 24408883 | C/G |
| 7 | 24422904 | A/G |
| 7 | 24454050 | G/T |
| 7 | 24506943 | A/G |
| 7 | 24517814 | C/A |
| 7 | 24545403 | C/A |
| 7 | 24690319 | G/A |
| 7 | 24704191 | G/A |
| 7 | 24811543 | T/C |
| 7 | 24867355 | C/T |
| 7 | 24880396 | T/C |
| 7 | 24889446 | C/T |
| 7 | 24922362 | T/A |
| 7 | 24950612 | C/T |
| 7 | 24954583 | A/G |
| 7 | 24978495 | G/A |
| 7 | 25015888 | A/G |
| 7 | 25024649 | C/T |
| 7 | 25069049 | T/C |
| 7 | 25075460 | T/C |
| 7 | 25090647 | T/C |
| 7 | 25116046 | G/A |
| 7 | 25119575 | C/G |
| 7 | 25158815 | C/G |
| 7 | 25168397 | C/T |
| 7 | 25170880 | T/A |
| 7 | 25248338 | C/G |
| 7 | 25267218 | T/A |
| 7 | 25293802 | G/A |
| 7 | 25295901 | G/A |
| 7 | 25349332 | G/A |
| 7 | 25355475 | G/A |
| 7 | 25378374 | A/T |
| 7 | 25448379 | T/C |
| 7 | 25469867 | T/A |
| 7 | 25493288 | G/A |
| 7 | 25508710 | C/A |
| 7 | 25554995 | T/G |
| 7 | 25558111 | T/C |
| 7 | 25577379 | G/A |
| 7 | 25607432 | A/G |
| 7 | 25616642 | A/G |
| 7 | 25627942 | T/C |
| 7 | 25642188 | T/C |
| 7 | 25655466 | T/A |
| 7 | 25677217 | G/C |
| 7 | 25689103 | T/C |
| 7 | 25702225 | G/T |
| 7 | 25733022 | A/G |
| 7 | 25741782 | A/G |
| 7 | 25792886 | T/C |
| 7 | 25803799 | T/C |
| 7 | 25810392 | G/A |
| 7 | 25842847 | C/G |
| 7 | 25859323 | A/G |
| 7 | 25864905 | C/T |
| 7 | 25918077 | G/A |
| 7 | 25944429 | C/T |
| 7 | 25971967 | C/T |
| 7 | 25973172 | T/C |
| 7 | 26015489 | C/T |
| 7 | 26061738 | C/A |
| 7 | 26069302 | G/C |
| 7 | 26074631 | T/A |
| 7 | 26079219 | A/C |
| 7 | 26127341 | C/T |
| 7 | 26129522 | A/T |
| 7 | 26130614 | T/C |
| 7 | 26162346 | T/C |
| 7 | 26179815 | A/C |
| 7 | 26245959 | A/C |
| 7 | 26276002 | C/A |
| 7 | 26277164 | A/G |
| 7 | 26289313 | A/G |
| 7 | 26323527 | A/G |
| 7 | 26332336 | C/T |
| 7 | 26342269 | A/C |
| 7 | 26367676 | G/A |
| 7 | 26372993 | C/T |
| 7 | 26411235 | T/C |
| 7 | 26417106 | C/A |
| 7 | 26430986 | C/G |
| 7 | 26462571 | G/A |
| 7 | 26481736 | C/A |
| 7 | 26516041 | T/C |
| 7 | 26548178 | A/T |
| 7 | 26614163 | G/A |
| 7 | 26651119 | C/A |
| 7 | 26675615 | T/C |
| 7 | 26714299 | A/T |
| 7 | 26716407 | G/A |
| 7 | 26819999 | C/T |
| 7 | 26827028 | A/G |
| 7 | 26830972 | C/T |
| 7 | 26853597 | A/G |
| 7 | 26863639 | C/G |
| 7 | 26894421 | G/A |
| 7 | 26920369 | C/G |
| 7 | 26929492 | G/A |
| 7 | 26974973 | G/A |
| 7 | 26979234 | G/A |
| 7 | 27007663 | G/A |
| 7 | 27072006 | A/G |
| 7 | 27089458 | A/G |
| 7 | 27165240 | C/T |
| 7 | 27174431 | G/A |
| 7 | 27225028 | A/T |
| 7 | 27250147 | T/C |
| 7 | 27300729 | C/T |
| 7 | 27349963 | C/G |
| 7 | 27388549 | T/C |
| 7 | 27440578 | T/C |
| 7 | 27454954 | A/T |
| 7 | 27462863 | C/T |
| 7 | 27480750 | G/A |
| 7 | 27829753 | G/A |
| 7 | 27864327 | C/G |
| 7 | 27879709 | A/T |
| 7 | 27926775 | A/G |
| 7 | 27946611 | G/C |
| 7 | 27957076 | A/G |
| 7 | 27986883 | A/G |
| 7 | 27988715 | C/A |
| 7 | 27993639 | G/C |
| 7 | 28022820 | C/G |
| 7 | 28085165 | A/T |
| 7 | 28094890 | A/T |
| 7 | 28140966 | G/A |
| 7 | 28208169 | A/C |
| 7 | 28266893 | T/C |
| 7 | 28319515 | C/T |
| 7 | 28348165 | T/A |
| 7 | 28366792 | C/A |
| 7 | 28400622 | G/A |
| 7 | 28433773 | A/G |
| 7 | 28437426 | G/A |
| 7 | 28469932 | A/G |
| 7 | 28490432 | C/G |
| 7 | 28498646 | G/A |
| 7 | 28522592 | A/G |
| 7 | 28583822 | A/T |
| 7 | 28593199 | T/C |
| 7 | 28594978 | T/C |
| 7 | 28623989 | G/A |
| 7 | 28654920 | G/A |
| 7 | 28657161 | G/A |
| 7 | 28714583 | G/A |
| 7 | 28741298 | C/T |
| 7 | 28745608 | A/C |
| 7 | 28770278 | C/T |
| 7 | 28798958 | A/G |
| 7 | 28823616 | T/C |
| 7 | 28829671 | C/T |
| 7 | 28834155 | G/C |
| 7 | 28835954 | C/T |
| 7 | 28863112 | A/C |
| 7 | 28876830 | A/C |
| 7 | 28897138 | C/T |
| 7 | 28916840 | T/C |
| 7 | 28923022 | A/G |
| 7 | 28957023 | G/A |
| 7 | 28994301 | T/G |
| 7 | 29003424 | T/A |
| 7 | 29012624 | C/G |
| 7 | 29031792 | C/T |
| 7 | 29054822 | T/G |
| 7 | 29063144 | A/G |
| 7 | 29073230 | T/C |
| 7 | 29126354 | C/T |
| 7 | 29128793 | G/A |
| 7 | 29139206 | A/G |
| 7 | 29179675 | T/A |
| 7 | 29194151 | T/C |
| 7 | 29236019 | G/T |
| 7 | 29248147 | T/C |
| 7 | 29255422 | T/G |
| 7 | 29270773 | T/G |
| 7 | 29286006 | T/A |
| 7 | 29293401 | C/T |
| 7 | 29331491 | T/C |
| 7 | 29344683 | T/G |
| 7 | 29363216 | C/T |
| 7 | 29379704 | C/T |
| 7 | 29400553 | G/A |
| 7 | 29430028 | C/T |
| 7 | 29452656 | C/A |
| 7 | 29471783 | A/G |
| 7 | 29519490 | C/T |
| 7 | 29544412 | G/C |
| 7 | 29571984 | A/C |
| 7 | 29590552 | C/T |
| 7 | 29611421 | T/A |
| 7 | 29631674 | A/G |
| 7 | 29654579 | T/C |
| 7 | 29668113 | A/C |
| 7 | 29675701 | C/A |
| 7 | 29677564 | T/A |
| 7 | 29701321 | C/T |
| 7 | 29705395 | T/G |
| 7 | 29730185 | G/A |
| 7 | 29773457 | C/T |
| 7 | 29776075 | C/A |
| 7 | 29796382 | T/C |
| 7 | 29803587 | A/T |
| 7 | 29828644 | C/G |
| 7 | 29847637 | T/A |
| 7 | 29864828 | T/C |
| 7 | 29872776 | A/G |
| 7 | 29884994 | C/T |
| 7 | 29917150 | A/C |
| 7 | 29920139 | T/A |
| 7 | 30004004 | A/C |
| 7 | 30022983 | C/A |
| 7 | 30051652 | G/T |
| 7 | 30061160 | A/C |
| 7 | 30063754 | A/T |
| 7 | 30097193 | T/C |
| 7 | 30100826 | G/A |
| 7 | 30127018 | G/C |
| 7 | 30150151 | T/A |
| 7 | 30171025 | C/A |
| 7 | 30205382 | T/C |
| 7 | 30226574 | T/A |
| 7 | 30237172 | C/T |
| 7 | 30251846 | G/A |
| 7 | 30253848 | G/A |
| 7 | 30270996 | T/A |
| 7 | 30305406 | G/C |
| 7 | 30307692 | A/G |
| 7 | 30352882 | C/T |
| 7 | 30361384 | C/T |
| 7 | 30366290 | A/G |
| 7 | 30390484 | G/A |
| 7 | 30444513 | C/T |
| 7 | 30461698 | T/G |
| 7 | 30483344 | A/G |
| 7 | 30484666 | T/C |
| 7 | 30527764 | C/T |
| 7 | 30611560 | G/C |
| 7 | 30627939 | A/G |
| 7 | 30633697 | A/G |
| 7 | 30661423 | A/T |
| 7 | 30673263 | C/T |
| 7 | 30715903 | G/T |
| 7 | 30728303 | A/T |
| 7 | 30735044 | C/G |
| 7 | 30771376 | A/C |
| 7 | 30778261 | G/A |
| 7 | 30782389 | A/T |
| 7 | 30828910 | T/A |
| 7 | 30925162 | G/C |
| 7 | 30928522 | A/T |
| 7 | 30930723 | T/A |
| 7 | 30960802 | T/G |
| 7 | 31006770 | G/A |
| 7 | 31020876 | G/A |
| 7 | 31037265 | T/C |
| 7 | 31058903 | A/T |
| 7 | 31077298 | T/C |
| 7 | 31107983 | A/C |
| 7 | 31113584 | C/T |
| 7 | 31119598 | G/A |
| 7 | 31147848 | G/A |
| 7 | 31192949 | C/G |
| 7 | 31211459 | G/T |
| 7 | 31236786 | C/T |
| 7 | 31241846 | G/A |
| 7 | 31264060 | T/C |
| 7 | 31286019 | T/C |
| 7 | 31293882 | G/A |
| 7 | 31339059 | A/T |
| 7 | 31369477 | C/G |
| 7 | 31371058 | A/G |
| 7 | 31380163 | T/G |
| 7 | 31398810 | A/G |
| 7 | 31420955 | G/C |
| 7 | 31432948 | C/G |
| 7 | 31453389 | A/C |
| 7 | 31458036 | G/A |
| 7 | 31499553 | A/C |
| 7 | 31512021 | T/C |
| 7 | 31528223 | T/G |
| 7 | 31555554 | G/A |
| 7 | 31564431 | A/G |
| 7 | 31607274 | G/C |
| 7 | 31628825 | A/C |
| 7 | 31630441 | T/G |
| 7 | 31673042 | C/G |
| 7 | 31681124 | G/T |
| 7 | 31702187 | G/A |
| 7 | 31725495 | A/G |
| 7 | 31739944 | T/C |
| 7 | 31757732 | G/A |
| 7 | 31796985 | A/G |
| 7 | 31801992 | T/G |
| 7 | 31842535 | A/T |
| 7 | 31881574 | A/G |
| 7 | 31889403 | C/T |
| 7 | 31891851 | G/T |
| 7 | 31900015 | A/C |
| 7 | 31937038 | C/T |
| 7 | 31955277 | T/A |
| 7 | 31981630 | A/T |
| 7 | 32038484 | C/T |
| 7 | 32058788 | G/A |
| 7 | 32061073 | T/A |
| 7 | 32073645 | C/G |
| 7 | 32101058 | T/C |
| 7 | 32110561 | G/A |
| 7 | 32158072 | T/G |
| 7 | 32160105 | C/T |
| 7 | 32208081 | C/T |
| 7 | 32216417 | G/A |
| 7 | 32229546 | C/G |
| 7 | 32262594 | C/T |
| 7 | 32278786 | A/T |
| 7 | 32303610 | G/A |
| 7 | 32323972 | G/A |
| 7 | 32348219 | T/A |
| 7 | 32367517 | C/T |
| 7 | 32383165 | T/C |
| 7 | 32396631 | T/C |
| 7 | 32420867 | A/G |
| 7 | 32435042 | C/A |
| 7 | 32468897 | T/C |
| 7 | 32472617 | T/A |
| 7 | 32494962 | C/A |
| 7 | 32497133 | A/G |
| 7 | 32533258 | T/C |
| 7 | 32576820 | T/C |
| 7 | 32579816 | G/A |
| 7 | 32584384 | A/G |
| 7 | 32594272 | T/A |
| 7 | 32612830 | G/A |
| 7 | 32645320 | T/C |
| 7 | 32646751 | C/T |
| 7 | 32689661 | G/A |
| 7 | 32692285 | G/A |
| 7 | 32703987 | C/T |
| 7 | 32728624 | A/T |
| 7 | 32770333 | A/G |
| 7 | 32804106 | T/C |
| 7 | 32813756 | A/G |
| 7 | 32848378 | A/T |
| 7 | 32894222 | C/A |
| 7 | 32934599 | C/T |
| 7 | 32940393 | C/A |
| 7 | 32941812 | A/T |
| 7 | 32975968 | T/C |
| 7 | 32985118 | G/C |
| 7 | 33003668 | C/T |
| 7 | 33010372 | G/A |
| 7 | 33056371 | C/G |
| 7 | 33061142 | C/T |
| 7 | 33097513 | T/C |
| 7 | 33118638 | A/G |
| 7 | 33122539 | C/T |
| 7 | 33140306 | C/T |
| 7 | 33151398 | T/C |
| 7 | 33161329 | C/G |
| 7 | 33180948 | T/C |
| 7 | 33182497 | C/T |
| 7 | 33217046 | T/A |
| 7 | 33245714 | C/T |
| 7 | 33269229 | C/T |
| 7 | 33284887 | C/G |
| 7 | 33301861 | A/T |
| 7 | 33324633 | G/T |
| 7 | 33339491 | A/G |
| 7 | 33350404 | G/A |
| 7 | 33370893 | A/G |
| 7 | 33373979 | G/A |
| 7 | 33400514 | T/C |
| 7 | 33423707 | G/A |
| 7 | 33439664 | G/A |
| 7 | 33455114 | C/T |
| 7 | 33463689 | C/G |
| 7 | 33498511 | C/T |
| 7 | 33529970 | G/A |
| 7 | 33546686 | G/A |
| 7 | 33596421 | C/G |
| 7 | 33616632 | C/T |
| 7 | 33645469 | G/A |
| 7 | 33647294 | A/C |
| 7 | 33674613 | G/T |
| 7 | 33713828 | G/A |
| 7 | 33728852 | G/A |
| 7 | 33733135 | G/A |
| 7 | 33763258 | G/T |
| 7 | 33781333 | A/G |
| 7 | 33783303 | T/A |
| 7 | 33813272 | T/G |
| 7 | 33815726 | C/G |
| 7 | 33860140 | A/C |
| 7 | 33862905 | C/T |
| 7 | 33885806 | G/T |
| 7 | 33924156 | T/C |
| 7 | 33929467 | A/T |
| 7 | 33930729 | C/A |
| 7 | 33940734 | A/G |
| 7 | 33970973 | A/C |
| 7 | 33988710 | T/C |
| 7 | 34039393 | T/G |
| 7 | 34056361 | G/A |
| 7 | 34091517 | T/C |
| 7 | 34094904 | A/G |
| 7 | 34111244 | C/T |
| 7 | 34148354 | T/C |
| 7 | 34152439 | T/C |
| 7 | 34173493 | G/A |
| 7 | 34195608 | G/A |
| 7 | 34214207 | T/C |
| 7 | 34254416 | C/T |
| 7 | 34266436 | C/T |
| 7 | 34287239 | A/T |
| 7 | 34294576 | T/C |
| 7 | 34296499 | A/T |
| 7 | 34335428 | G/A |
| 7 | 34351368 | T/C |
| 7 | 34374600 | C/T |
| 7 | 34385846 | C/T |
| 7 | 34393181 | C/T |
| 7 | 34412221 | G/A |
| 7 | 34431431 | G/A |
| 7 | 34443680 | T/A |
| 7 | 34446421 | A/T |
| 7 | 34470052 | C/A |
| 7 | 34474244 | A/G |
| 7 | 34511419 | T/C |
| 7 | 34527318 | C/A |
| 7 | 34536432 | T/C |
| 7 | 34561100 | A/G |
| 7 | 34584108 | A/G |
| 7 | 34608239 | C/A |
| 7 | 34618481 | C/T |
| 7 | 34626146 | A/G |
| 7 | 34650404 | C/T |
| 7 | 34664024 | T/A |
| 7 | 34664025 | C/T |
| 7 | 34664026 | C/T |
| 7 | 34664027 | C/T |
| 7 | 34664028 | A/G |
| 7 | 34664029 | A/G |
| 7 | 34664030 | A/G |
| 7 | 34664031 | T/C |
| 7 | 34664032 | T/C |
| 7 | 34664033 | T/C |
| 7 | 34744194 | C/T |
| 7 | 34744195 | G/A |
| 7 | 34744196 | G/A |
| 7 | 34744197 | G/A |
| 7 | 34744198 | A/G |
| 7 | 34744199 | G/A |
| 7 | 34744200 | G/A |
| 7 | 34744201 | G/A |
| 7 | 34836337 | T/G |
| 7 | 34849262 | A/G |
| 7 | 34849263 | C/T |
| 7 | 34849264 | C/T |
| 7 | 34849265 | A/T |
| 7 | 34849266 | A/T |
| 7 | 34849267 | A/T |
| 7 | 34892862 | T/C |
| 7 | 34918155 | G/A |
| 7 | 34937264 | A/G |
| 7 | 34946239 | G/T |
| 7 | 34958982 | T/A |
| 7 | 34976675 | T/A |
| 7 | 35001716 | A/G |
| 7 | 35002876 | A/G |
| 7 | 35021178 | T/C |
| 7 | 35041733 | G/T |
| 7 | 35055324 | T/A |
| 7 | 35071455 | C/T |
| 7 | 35103859 | C/T |
| 7 | 35123218 | T/A |
| 7 | 35133619 | C/G |
| 7 | 35151161 | A/G |
| 7 | 35160160 | T/A |
| 7 | 35184632 | T/C |
| 7 | 35202100 | C/A |
| 7 | 35203279 | C/T |
| 7 | 35222071 | A/G |
| 7 | 35254649 | A/G |
| 7 | 35275917 | C/T |
| 7 | 35296635 | G/A |
| 7 | 35336783 | A/G |
| 7 | 35352100 | A/T |
| 7 | 35354385 | T/C |
| 7 | 35380933 | A/G |
| 7 | 35414405 | C/T |
| 7 | 35417066 | T/C |
| 7 | 35440921 | T/C |
| 7 | 35474995 | G/T |
| 7 | 35495782 | C/T |
| 7 | 35529239 | T/A |
| 7 | 35532736 | C/G |
| 7 | 35574972 | C/A |
| 7 | 35576756 | T/C |
| 7 | 35580135 | C/T |
| 7 | 35610261 | A/C |
| 7 | 35666829 | C/T |
| 7 | 35669679 | C/T |
| 7 | 35672394 | A/G |
| 7 | 35706371 | C/T |
| 7 | 35733849 | C/T |
| 7 | 35746104 | A/G |
| 7 | 35774151 | T/A |
| 7 | 35837646 | A/G |
| 7 | 35871353 | C/A |
| 7 | 35884295 | A/G |
| 7 | 35894525 | G/T |
| 7 | 35915224 | T/A |
| 7 | 35962228 | T/C |
| 7 | 35973142 | C/T |
| 7 | 35987569 | T/C |
| 7 | 36012568 | G/T |
| 7 | 36029998 | A/G |
| 7 | 36051493 | A/C |
| 7 | 36062495 | T/C |
| 7 | 36084740 | A/G |
| 7 | 36096726 | A/T |
| 7 | 36121704 | T/C |
| 7 | 36130749 | T/G |
| 7 | 36156405 | A/G |
| 7 | 36162601 | C/G |
| 7 | 36184247 | A/G |
| 7 | 36186023 | G/A |
| 7 | 36216742 | T/A |
| 7 | 36231678 | T/C |
| 7 | 36253717 | G/A |
| 7 | 36255357 | C/G |
| 7 | 36277973 | A/G |
| 7 | 36290468 | G/T |
| 7 | 36308311 | T/C |
| 7 | 36340724 | G/T |
| 7 | 36360602 | T/C |
| 7 | 36368801 | C/T |
| 7 | 36398161 | A/C |
| 7 | 36411071 | A/G |
| 7 | 36430944 | C/T |
| 7 | 36458329 | C/T |
| 7 | 36460374 | C/A |
| 7 | 36485555 | G/C |
| 7 | 36487941 | G/T |
| 7 | 36513990 | T/C |
| 7 | 36544494 | A/G |
| 7 | 36576111 | T/C |
| 7 | 36582619 | G/A |
| 7 | 36604317 | C/G |
| 7 | 36606161 | C/A |
| 7 | 36641893 | G/A |
| 7 | 36653517 | C/T |
| 7 | 36677657 | A/G |
| 7 | 36689244 | A/C |
| 7 | 36698359 | C/G |
| 7 | 36701659 | A/G |
| 7 | 36738013 | A/G |
| 7 | 36749252 | A/G |
| 7 | 36752233 | G/C |
| 7 | 36768941 | C/T |
| 7 | 36780136 | C/G |
| 7 | 36793708 | C/G |
| 7 | 36827065 | A/G |
| 7 | 36851527 | T/C |
| 7 | 36863540 | G/T |
| 7 | 36894431 | T/A |
| 7 | 36896426 | G/A |
| 7 | 36921062 | G/A |
| 7 | 36931731 | C/T |
| 7 | 36950031 | G/C |
| 7 | 36986910 | A/G |
| 7 | 36989003 | G/A |
| 7 | 36992612 | A/G |
| 7 | 37019764 | C/G |
| 7 | 37020900 | T/C |
| 7 | 37069698 | C/T |
| 7 | 37089616 | G/A |
| 7 | 37108544 | A/T |
| 7 | 37115189 | T/C |
| 7 | 37128848 | C/T |
| 7 | 37144421 | A/T |
| 7 | 37148963 | T/A |
| 7 | 37176583 | G/C |
| 7 | 37191821 | G/A |
| 7 | 37218372 | C/G |
| 7 | 37231978 | A/T |
| 7 | 37244790 | C/T |
| 7 | 37281790 | G/A |
| 7 | 37297166 | G/A |
| 7 | 37324240 | T/A |
| 7 | 37354568 | T/C |
| 7 | 37376490 | A/G |
| 7 | 37406055 | C/G |
| 7 | 37417960 | T/A |
| 7 | 37423360 | G/A |
| 7 | 37441573 | A/G |
| 7 | 37447686 | T/C |
| 7 | 37470346 | C/A |
| 7 | 37488057 | C/T |
| 7 | 37500598 | G/T |
| 7 | 37509334 | G/T |
| 7 | 37532149 | T/G |
| 7 | 37548596 | A/G |
| 7 | 37563809 | C/G |
| 7 | 37571757 | T/C |
| 7 | 37590390 | A/C |
| 7 | 37649261 | T/C |
| 7 | 37672566 | C/A |
| 7 | 37707170 | C/T |
| 7 | 37714327 | A/T |
| 7 | 37742316 | A/G |
| 7 | 37794192 | T/G |
| 7 | 37797906 | T/C |
| 7 | 37801092 | C/T |
| 7 | 37843727 | C/T |
| 7 | 37845017 | A/G |
| 8 | 19408 | C/T |
| 8 | 23617 | A/C |
| 8 | 52243 | C/T |
| 8 | 60514 | G/A |
| 8 | 116155 | G/A |
| 8 | 128706 | C/T |
| 8 | 131657 | G/A |
| 8 | 154059 | G/C |
| 8 | 184265 | C/A |
| 8 | 227607 | G/A |
| 8 | 255083 | A/T |
| 8 | 260443 | C/T |
| 8 | 288926 | T/G |
| 8 | 296692 | C/T |
| 8 | 303237 | T/G |
| 8 | 307352 | A/G |
| 8 | 342918 | G/A |
| 8 | 344528 | A/G |
| 8 | 367868 | G/A |
| 8 | 390375 | A/T |
| 8 | 410419 | A/G |
| 8 | 420102 | C/T |
| 8 | 465130 | A/G |
| 8 | 481980 | G/A |
| 8 | 497318 | T/C |
| 8 | 513205 | G/A |
| 8 | 514257 | C/T |
| 8 | 551748 | C/T |
| 8 | 565119 | T/C |
| 8 | 580170 | C/T |
| 8 | 597255 | G/A |
| 8 | 629281 | C/T |
| 8 | 639050 | A/G |
| 8 | 660760 | C/A |
| 8 | 661937 | A/G |
| 8 | 700330 | G/A |
| 8 | 726764 | C/T |
| 8 | 742536 | A/T |
| 8 | 773645 | G/A |
| 8 | 793007 | T/C |
| 8 | 804877 | G/A |
| 8 | 811214 | T/A |
| 8 | 840786 | T/C |
| 8 | 851052 | A/G |
| 8 | 871307 | T/C |
| 8 | 879588 | C/A |
| 8 | 914071 | C/T |
| 8 | 925573 | T/C |
| 8 | 940651 | A/G |
| 8 | 963711 | G/A |
| 8 | 979321 | T/A |
| 8 | 994112 | C/G |
| 8 | 1008246 | G/A |
| 8 | 1044304 | G/C |
| 8 | 1049058 | C/T |
| 8 | 1051151 | A/T |
| 8 | 1057022 | G/T |
| 8 | 1080202 | C/T |
| 8 | 1118303 | C/T |
| 8 | 1159419 | C/T |
| 8 | 1182240 | C/T |
| 8 | 1203265 | A/G |
| 8 | 1225820 | C/T |
| 8 | 1236116 | C/G |
| 8 | 1285443 | C/T |
| 8 | 1295835 | C/G |
| 8 | 1298688 | A/G |
| 8 | 1344497 | G/A |
| 8 | 1348715 | A/C |
| 8 | 1367671 | A/T |
| 8 | 1395056 | A/T |
| 8 | 1407162 | G/A |
| 8 | 1411666 | T/C |
| 8 | 1424427 | C/T |
| 8 | 1440384 | T/C |
| 8 | 1488055 | G/C |
| 8 | 1492901 | T/A |
| 8 | 1502297 | G/A |
| 8 | 1555443 | G/C |
| 8 | 1560587 | T/G |
| 8 | 1605705 | T/C |
| 8 | 1641601 | G/A |
| 8 | 1642698 | A/C |
| 8 | 1675872 | C/A |
| 8 | 1717522 | T/A |
| 8 | 1730310 | A/G |
| 8 | 1751640 | A/C |
| 8 | 1759581 | T/G |
| 8 | 1773847 | G/A |
| 8 | 1778218 | T/G |
| 8 | 1819137 | A/C |
| 8 | 1837218 | T/C |
| 8 | 1840215 | G/A |
| 8 | 1860472 | C/A |
| 8 | 1866449 | G/A |
| 8 | 1893252 | C/G |
| 8 | 1897184 | G/A |
| 8 | 1946676 | G/A |
| 8 | 1950834 | G/T |
| 8 | 1953216 | A/C |
| 8 | 1986266 | A/T |
| 8 | 2003397 | G/A |
| 8 | 2011641 | C/T |
| 8 | 2026531 | T/C |
| 8 | 2043474 | G/A |
| 8 | 2046143 | C/T |
| 8 | 2075120 | T/C |
| 8 | 2106214 | A/G |
| 8 | 2121449 | C/T |
| 8 | 2136903 | T/C |
| 8 | 2148795 | G/A |
| 8 | 2161929 | G/A |
| 8 | 2178619 | T/C |
| 8 | 2203236 | C/A |
| 8 | 2237722 | C/A |
| 8 | 2252916 | G/A |
| 8 | 2272903 | G/C |
| 8 | 2283492 | A/C |
| 8 | 2286849 | C/T |
| 8 | 2324564 | G/A |
| 8 | 2327630 | C/A |
| 8 | 2344733 | C/T |
| 8 | 2345876 | G/A |
| 8 | 2376970 | C/T |
| 8 | 2390284 | G/C |
| 8 | 2427906 | C/T |
| 8 | 2439737 | C/T |
| 8 | 2465040 | A/T |
| 8 | 2466478 | T/C |
| 8 | 2510420 | C/T |
| 8 | 2532252 | T/C |
| 8 | 2565100 | A/C |
| 8 | 2575812 | G/A |
| 8 | 2612785 | A/T |
| 8 | 2637349 | A/G |
| 8 | 2655361 | A/C |
| 8 | 2657596 | C/T |
| 8 | 2678383 | A/G |
| 8 | 2681433 | A/G |
| 8 | 2716611 | G/A |
| 8 | 2718792 | A/G |
| 8 | 2748537 | T/G |
| 8 | 2773500 | C/G |
| 8 | 2778670 | G/C |
| 8 | 2792235 | G/A |
| 8 | 2844847 | C/T |
| 8 | 2860689 | T/A |
| 8 | 2865428 | A/T |
| 8 | 2881317 | C/T |
| 8 | 2882622 | C/A |
| 8 | 2927537 | A/G |
| 8 | 2935510 | G/A |
| 8 | 2940389 | T/C |
| 8 | 2968705 | T/A |
| 8 | 2971225 | G/A |
| 8 | 2994523 | C/A |
| 8 | 3020066 | C/T |
| 8 | 3045893 | C/T |
| 8 | 3055923 | C/T |
| 8 | 3068288 | C/T |
| 8 | 3091450 | C/T |
| 8 | 3098692 | G/A |
| 8 | 3120437 | C/A |
| 8 | 3154016 | C/G |
| 8 | 3159084 | G/T |
| 8 | 3197312 | G/A |
| 8 | 3202054 | T/A |
| 8 | 3213874 | G/A |
| 8 | 3225711 | C/T |
| 8 | 3258303 | T/G |
| 8 | 3266455 | C/T |
| 8 | 3281170 | G/T |
| 8 | 3313364 | C/T |
| 8 | 3318786 | A/G |
| 8 | 3355265 | G/A |
| 8 | 3373278 | C/T |
| 8 | 3389855 | T/C |
| 8 | 3407398 | T/C |
| 8 | 3425953 | A/G |
| 8 | 3439672 | A/G |
| 8 | 3463972 | T/C |
| 8 | 3468993 | T/A |
| 8 | 3492782 | C/A |
| 8 | 3517280 | C/T |
| 8 | 3517281 | C/G |
| 8 | 3517282 | C/G |
| 8 | 3517283 | C/G |
| 8 | 3517284 | T/G |
| 8 | 3517285 | T/G |
| 8 | 3517286 | T/G |
| 8 | 3623125 | G/A |
| 8 | 3653125 | A/G |
| 8 | 3679361 | C/T |
| 8 | 3689199 | C/T |
| 8 | 3692640 | A/G |
| 8 | 3694406 | T/G |
| 8 | 3739989 | A/C |
| 8 | 3746415 | C/T |
| 8 | 3771408 | T/G |
| 8 | 3783308 | C/G |
| 8 | 3798677 | G/C |
| 8 | 3827851 | C/A |
| 8 | 3845242 | A/T |
| 8 | 3873637 | G/A |
| 8 | 3897627 | G/A |
| 8 | 3906156 | A/C |
| 8 | 3936551 | C/T |
| 8 | 3963828 | C/T |
| 8 | 3976591 | A/T |
| 8 | 3996628 | A/C |
| 8 | 4030476 | A/G |
| 8 | 4049016 | A/T |
| 8 | 4076534 | C/A |
| 8 | 4099968 | A/T |
| 8 | 4105439 | A/C |
| 8 | 4114393 | T/C |
| 8 | 4148611 | A/G |
| 8 | 4185043 | T/C |
| 8 | 4200118 | T/C |
| 8 | 4229530 | C/A |
| 8 | 4243590 | C/A |
| 8 | 4263696 | G/A |
| 8 | 4270156 | T/C |
| 8 | 4310435 | G/A |
| 8 | 4328310 | T/G |
| 8 | 4351613 | A/T |
| 8 | 4374653 | T/A |
| 8 | 4391383 | G/A |
| 8 | 4416057 | T/C |
| 8 | 4450870 | T/C |
| 8 | 4482260 | A/C |
| 8 | 4526720 | C/A |
| 8 | 4538144 | T/A |
| 8 | 4543126 | C/T |
| 8 | 4563348 | G/A |
| 8 | 4566038 | T/G |
| 8 | 4605787 | G/C |
| 8 | 4673737 | C/T |
| 8 | 4677636 | G/T |
| 8 | 4696303 | T/A |
| 8 | 4734458 | C/A |
| 8 | 4747380 | G/A |
| 8 | 4754618 | T/G |
| 8 | 4829105 | G/A |
| 8 | 4838135 | A/G |
| 8 | 4852180 | C/A |
| 8 | 4865448 | A/G |
| 8 | 4900237 | C/T |
| 8 | 4927618 | A/G |
| 8 | 4940297 | C/G |
| 8 | 4957441 | G/A |
| 8 | 4986999 | G/C |
| 8 | 5010173 | A/G |
| 8 | 5052774 | G/C |
| 8 | 5056180 | G/A |
| 8 | 5072806 | T/C |
| 8 | 5111764 | T/C |
| 8 | 5122097 | G/A |
| 8 | 5142710 | A/G |
| 8 | 5185469 | T/A |
| 8 | 5197091 | G/C |
| 8 | 5203456 | A/G |
| 8 | 5226162 | G/A |
| 8 | 5243621 | C/T |
| 8 | 5264403 | C/A |
| 8 | 5265416 | G/A |
| 8 | 5280566 | C/T |
| 8 | 5294109 | G/A |
| 8 | 5330108 | C/T |
| 8 | 5332880 | G/C |
| 8 | 5343289 | G/A |
| 8 | 5344569 | G/A |
| 8 | 5374050 | A/C |
| 8 | 5389689 | G/A |
| 8 | 5430018 | C/T |
| 8 | 5431394 | A/C |
| 8 | 5461590 | T/C |
| 8 | 5475859 | T/G |
| 8 | 5496521 | G/A |
| 8 | 5507181 | C/A |
| 8 | 5532610 | C/T |
| 8 | 5545723 | G/A |
| 8 | 5578103 | T/C |
| 8 | 5594558 | G/A |
| 8 | 5635456 | A/G |
| 8 | 5641001 | G/C |
| 8 | 5670994 | A/G |
| 8 | 5719379 | A/T |
| 8 | 5723038 | T/C |
| 8 | 5732941 | T/G |
| 8 | 5771776 | C/T |
| 8 | 5773529 | A/G |
| 8 | 5802263 | A/G |
| 8 | 5823669 | G/A |
| 8 | 5838056 | A/T |
| 8 | 5850803 | A/G |
| 8 | 5864841 | C/T |
| 8 | 5897593 | C/A |
| 8 | 5898621 | C/T |
| 8 | 5914090 | C/T |
| 8 | 5919664 | T/A |
| 8 | 5951357 | A/G |
| 8 | 5955257 | T/C |
| 8 | 5990353 | C/G |
| 8 | 6007882 | A/G |
| 8 | 6057367 | T/C |
| 8 | 6059096 | A/G |
| 8 | 6065762 | G/A |
| 8 | 6079826 | G/A |
| 8 | 6104838 | A/C |
| 8 | 6113272 | C/T |
| 8 | 6135398 | C/A |
| 8 | 6156225 | G/A |
| 8 | 6192246 | A/G |
| 8 | 6212703 | C/T |
| 8 | 6214356 | A/G |
| 8 | 6246003 | A/G |
| 8 | 6263166 | C/T |
| 8 | 6274561 | C/T |
| 8 | 6275620 | T/C |
| 8 | 6303977 | A/G |
| 8 | 6351178 | A/G |
| 8 | 6357049 | C/T |
| 8 | 6382557 | G/C |
| 8 | 6408844 | A/G |
| 8 | 6421622 | T/C |
| 8 | 6443428 | T/C |
| 8 | 6468024 | A/C |
| 8 | 6494926 | A/G |
| 8 | 6515056 | A/G |
| 8 | 6557029 | C/G |
| 8 | 6559479 | C/A |
| 8 | 6579564 | G/T |
| 8 | 6594475 | G/A |
| 8 | 6614360 | C/T |
| 8 | 6658612 | G/A |
| 8 | 6670765 | G/C |
| 8 | 6672260 | T/C |
| 8 | 6696820 | T/A |
| 8 | 6711010 | C/T |
| 8 | 6735394 | C/T |
| 8 | 3739647 | G/A |
| 8 | 6751263 | C/T |
| 8 | 6780916 | C/T |
| 8 | 6809994 | T/A |
| 8 | 6813027 | A/G |
| 8 | 6845678 | C/T |
| 8 | 6865875 | C/T |
| 8 | 6877092 | A/G |
| 8 | 6879094 | G/A |
| 8 | 6926551 | A/G |
| 8 | 6928010 | C/T |
| 8 | 6940468 | T/C |
| 8 | 6982090 | T/G |
| 8 | 7018998 | G/A |
| 8 | 7031108 | A/T |
| 8 | 7037289 | C/T |
| 8 | 7061631 | T/C |
| 8 | 7081261 | T/C |
| 8 | 7101359 | C/G |
| 8 | 7133181 | G/T |
| 8 | 7156900 | G/A |
| 8 | 7159996 | T/A |
| 8 | 7170896 | C/G |
| 8 | 7189813 | G/A |
| 8 | 7208055 | A/T |
| 8 | 7231265 | A/G |
| 8 | 7260902 | C/A |
| 8 | 7270823 | T/C |
| 8 | 7311004 | C/G |
| 8 | 7335651 | T/G |
| 8 | 7337568 | T/C |
| 8 | 7355022 | A/T |
| 8 | 7358720 | T/C |
| 8 | 7407939 | T/C |
| 8 | 7427276 | T/G |
| 8 | 7433265 | T/G |
| 8 | 7466586 | T/C |
| 8 | 7468240 | C/T |
| 8 | 7481415 | A/G |
| 8 | 7495436 | T/C |
| 8 | 7507469 | A/C |
| 8 | 7553589 | C/T |
| 8 | 7577642 | G/T |
| 8 | 7607433 | A/G |
| 8 | 7615119 | A/T |
| 8 | 7624198 | A/G |
| 8 | 7641066 | G/A |
| 8 | 7658648 | G/A |
| 8 | 7685709 | C/T |
| 8 | 7701967 | C/T |
| 8 | 7716298 | C/G |
| 8 | 7764072 | T/G |
| 8 | 7769856 | G/T |
| 8 | 7774249 | G/A |
| 8 | 7816435 | A/T |
| 8 | 7828470 | T/C |
| 8 | 7830208 | G/T |
| 8 | 7879542 | C/T |
| 8 | 7881571 | T/C |
| 8 | 7910821 | C/T |
| 8 | 7914660 | C/T |
| 8 | 7939962 | G/A |
| 8 | 7942702 | C/T |
| 8 | 7958126 | A/G |
| 8 | 7973919 | C/G |
| 8 | 7984826 | T/C |
| 8 | 8024130 | C/T |
| 8 | 8059129 | T/A |
| 8 | 8085735 | C/T |
| 8 | 8092402 | G/A |
| 8 | 8121412 | C/T |
| 8 | 8136892 | C/T |
| 8 | 8145959 | A/T |
| 8 | 8175296 | C/T |
| 8 | 8190381 | A/G |
| 8 | 8193015 | G/A |
| 8 | 8239034 | T/C |
| 8 | 8277677 | C/T |
| 8 | 8279990 | G/A |
| 8 | 8286385 | G/A |
| 8 | 8322243 | C/A |
| 8 | 8337085 | C/T |
| 8 | 8343615 | C/T |
| 8 | 8362541 | C/T |
| 8 | 8394033 | C/T |
| 8 | 8400594 | T/C |
| 8 | 8440323 | C/T |
| 8 | 8466640 | C/A |
| 8 | 8468589 | C/T |
| 8 | 8511942 | G/A |
| 8 | 8528980 | G/T |
| 8 | 8553502 | T/C |
| 8 | 8565362 | C/T |
| 8 | 8595075 | G/A |
| 8 | 8614180 | A/T |
| 8 | 8618663 | G/A |
| 8 | 8646532 | T/A |
| 8 | 8453562 | C/T |
| 8 | 8684390 | C/G |
| 8 | 8688021 | T/C |
| 8 | 8708096 | G/A |
| 8 | 8710245 | A/T |
| 8 | 8752133 | G/A |
| 8 | 8761218 | C/A |
| 8 | 8764356 | C/G |
| 8 | 8800436 | A/G |
| 8 | 8820635 | T/C |
| 8 | 8871833 | A/G |
| 8 | 8873115 | A/G |
| 8 | 8883126 | G/C |
| 8 | 8907446 | C/T |
| 8 | 8936892 | C/T |
| 8 | 8950132 | T/C |
| 8 | 8959799 | C/T |
| 8 | 8999363 | G/C |
| 8 | 9001393 | C/T |
| 8 | 9062308 | G/T |
| 8 | 9093908 | A/G |
| 8 | 9126168 | G/C |
| 8 | 9141447 | C/T |
| 8 | 9160751 | A/T |
| 8 | 9204862 | G/A |
| 8 | 9212202 | G/A |
| 8 | 9226686 | T/C |
| 8 | 9241051 | G/A |
| 8 | 9252595 | A/C |
| 8 | 9281822 | T/G |
| 8 | 9283992 | T/C |
| 8 | 9313330 | T/A |
| 8 | 9324598 | A/G |
| 8 | 9332675 | A/G |
| 8 | 9367638 | T/A |
| 8 | 9405463 | T/C |
| 8 | 9413977 | G/A |
| 8 | 9446111 | T/C |
| 8 | 9475037 | G/A |
| 8 | 9490023 | T/C |
| 8 | 9509681 | A/G |
| 8 | 9511120 | G/A |
| 8 | 9550193 | G/C |
| 8 | 9568532 | T/C |
| 8 | 9572889 | G/A |
| 8 | 9607068 | C/T |
| 8 | 9613371 | T/C |
| 8 | 9646726 | G/T |
| 8 | 9669978 | A/T |
| 8 | 9683607 | T/C |
| 8 | 9690671 | C/G |
| 8 | 9729415 | C/T |
| 8 | 9753284 | A/C |
| 8 | 9791348 | G/A |
| 8 | 9813617 | T/C |
| 8 | 9838934 | C/T |
| 8 | 9845813 | A/G |
| 8 | 9847431 | A/G |
| 8 | 9879027 | A/T |
| 8 | 9894678 | A/G |
| 8 | 9900694 | A/G |
| 8 | 9927276 | C/T |
| 8 | 9956114 | G/A |
| 8 | 9963514 | T/C |
| 8 | 9990518 | A/G |
| 8 | 10037071 | T/C |
| 8 | 10043528 | T/A |
| 8 | 10068482 | G/A |
| 8 | 10105272 | C/T |
| 8 | 10114582 | T/G |
| 8 | 10133328 | G/A |
| 8 | 10150267 | G/A |
| 8 | 10159314 | A/T |
| 8 | 10170238 | G/A |
| 8 | 10184921 | G/A |
| 8 | 10216002 | T/C |
| 8 | 10218048 | T/C |
| 8 | 10240658 | C/A |
| 8 | 10248149 | G/A |
| 8 | 10266655 | C/T |
| 8 | 10267994 | G/A |
| 8 | 10312726 | C/G |
| 8 | 10325811 | T/G |
| 8 | 10336126 | G/A |
| 8 | 10352852 | G/A |
| 8 | 10404272 | C/T |
| 8 | 10428326 | G/A |
| 8 | 10438398 | A/G |
| 8 | 10440825 | A/G |
| 8 | 10458000 | C/G |
| 8 | 10474282 | A/G |
| 8 | 10476201 | T/C |
| 8 | 10512885 | C/G |
| 8 | 10517667 | G/A |
| 8 | 10531954 | A/G |
| 8 | 10540449 | G/A |
| 8 | 10576859 | A/C |
| 8 | 10606725 | C/T |
| 8 | 10617886 | G/A |
| 8 | 10625592 | T/C |
| 8 | 10630719 | C/A |
| 8 | 10668797 | G/A |
| 8 | 10680550 | T/C |
| 8 | 10683068 | T/G |
| 8 | 10722766 | C/T |
| 8 | 10734196 | T/C |
| 8 | 10750738 | A/T |
| 8 | 10768847 | C/T |
| 8 | 10781227 | A/T |
| 8 | 10782557 | T/A |
| 8 | 10801303 | C/T |
| 8 | 10842908 | C/T |
| 8 | 10850935 | C/T |
| 8 | 10872988 | G/T |
| 8 | 10891715 | A/C |
| 8 | 10919205 | G/A |
| 8 | 10921110 | A/G |
| 8 | 10936506 | C/T |
| 8 | 10950060 | G/A |
| 8 | 10962937 | T/C |
| 8 | 10994393 | C/T |
| 8 | 11009595 | G/A |
| 8 | 11012480 | A/G |
| 8 | 11048850 | A/C |
| 8 | 11083877 | C/T |
| 8 | 11105221 | T/C |
| 8 | 11144118 | A/C |
| 8 | 11149295 | A/G |
| 8 | 11178539 | T/C |
| 8 | 11208077 | G/A |
| 8 | 11234301 | G/A |
| 8 | 11261421 | C/T |
| 8 | 11266208 | C/T |
| 8 | 11283205 | T/G |
| 8 | 11293990 | C/A |
| 8 | 11327252 | T/C |
| 8 | 11334697 | G/T |
| 8 | 11357816 | C/T |
| 8 | 11366147 | C/T |
| 8 | 11370731 | G/A |
| 8 | 11407384 | T/C |
| 8 | 11417433 | A/C |
| 8 | 11436317 | C/T |
| 8 | 11445927 | C/T |
| 8 | 11460177 | A/C |
| 8 | 11478560 | C/T |
| 8 | 11492953 | G/A |
| 8 | 11502451 | A/G |
| 8 | 11522981 | T/C |
| 8 | 11572677 | T/C |
| 8 | 11584714 | A/G |
| 8 | 11587108 | T/C |
| 8 | 11610881 | T/C |
| 8 | 11614557 | G/A |
| 8 | 11646409 | C/T |
| 8 | 11676020 | T/C |
| 8 | 11684057 | A/G |
| 8 | 11729988 | A/G |
| 8 | 11755889 | C/T |
| 8 | 11760011 | G/A |
| 8 | 11781378 | G/T |
| 8 | 11791333 | A/G |
| 8 | 11803233 | A/G |
| 8 | 11820667 | A/G |
| 8 | 11833301 | G/A |
| 8 | 11853116 | T/C |
| 8 | 11854788 | C/G |
| 8 | 11908954 | T/C |
| 8 | 11929691 | T/G |
| 8 | 11932206 | A/G |
| 8 | 11952228 | A/T |
| 8 | 11968343 | A/G |
| 8 | 11984105 | A/G |
| 8 | 12019155 | G/A |
| 8 | 12058529 | C/T |
| 8 | 12069417 | A/C |
| 8 | 12117844 | C/T |
| 8 | 12131781 | T/C |
| 8 | 12161793 | G/A |
| 8 | 12209113 | G/A |
| 8 | 12223166 | T/A |
| 8 | 12260555 | G/A |
| 8 | 12289511 | C/T |
| 8 | 12298527 | T/C |
| 8 | 12302728 | T/C |
| 8 | 12323029 | T/C |
| 8 | 12351753 | A/G |
| 8 | 12352804 | G/A |
| 8 | 12367339 | G/T |
| 8 | 12372552 | G/A |
| 8 | 12403196 | T/A |
| 8 | 12415876 | C/T |
| 8 | 12420122 | A/G |
| 8 | 12435612 | G/T |
| 8 | 12458198 | C/T |
| 8 | 12487589 | G/C |
| 8 | 12490951 | G/A |
| 8 | 12534973 | T/A |
| 8 | 12538923 | C/T |
| 8 | 12547951 | G/A |
| 8 | 12554673 | G/C |
| 8 | 12571811 | T/A |
| 8 | 12615639 | T/G |
| 8 | 12636294 | G/A |
| 8 | 12658950 | C/T |
| 8 | 12660951 | T/C |
| 8 | 12662283 | A/G |
| 8 | 12691405 | T/A |
| 8 | 12728464 | C/T |
| 8 | 12732467 | A/G |
| 8 | 12769776 | T/C |
| 8 | 12801893 | A/T |
| 8 | 12829760 | G/C |
| 8 | 12872972 | T/A |
| 8 | 12891254 | A/C |
| 8 | 12909778 | T/C |
| 8 | 12959310 | A/G |
| 8 | 12975571 | C/T |
| 8 | 12980656 | T/C |
| 8 | 13016507 | A/C |
| 8 | 13018300 | C/T |
| 8 | 13021766 | A/T |
| 8 | 13050523 | A/G |
| 8 | 13053226 | A/G |
| 8 | 13094562 | A/T |
| 8 | 13131924 | T/C |
| 8 | 13156264 | A/G |
| 8 | 13175901 | G/A |
| 8 | 13190595 | A/G |
| 8 | 13202032 | A/G |
| 8 | 13219355 | A/G |
| 8 | 13238882 | C/G |
| 8 | 13252965 | T/C |
| 8 | 13287941 | G/T |
| 8 | 13289214 | A/G |
| 8 | 13299275 | G/C |
| 8 | 13301710 | C/G |
| 8 | 13325295 | G/A |
| 8 | 13360149 | G/A |
| 8 | 13382395 | C/G |
| 8 | 13423273 | C/T |
| 8 | 13439545 | T/C |
| 8 | 13447579 | A/G |
| 8 | 13456084 | C/A |
| 8 | 13481068 | T/A |
| 8 | 13495063 | A/T |
| 8 | 13504508 | T/A |
| 8 | 13515462 | C/T |
| 8 | 13537061 | C/G |
| 8 | 13544002 | C/T |
| 8 | 13561115 | T/C |
| 8 | 13563614 | T/G |
| 8 | 13644870 | A/G |
| 8 | 13651595 | G/A |
| 8 | 13666240 | G/C |
| 8 | 13708307 | C/T |
| 8 | 13727803 | T/C |
| 8 | 13734930 | A/G |
| 8 | 13743858 | C/G |
| 8 | 13838383 | A/G |
| 8 | 13862760 | C/A |
| 8 | 13863895 | T/A |
| 8 | 13894961 | A/G |
| 8 | 13923425 | T/C |
| 8 | 13928433 | A/G |
| 8 | 13974181 | T/C |
| 8 | 13990991 | G/A |
| 8 | 13999579 | T/C |
| 8 | 14013927 | A/G |
| 8 | 14040239 | G/A |
| 8 | 14059915 | C/A |
| 8 | 14071159 | T/C |
| 8 | 14076342 | C/G |
| 8 | 14110463 | C/T |
| 8 | 14112485 | G/A |
| 8 | 14141464 | G/C |
| 8 | 14142520 | C/G |
| 8 | 14212645 | A/G |
| 8 | 14256034 | T/A |
| 8 | 14260367 | T/C |
| 8 | 14301477 | T/C |
| 8 | 14306592 | T/G |
| 8 | 14319672 | T/C |
| 8 | 14335100 | T/C |
| 8 | 14365678 | G/A |
| 8 | 14422745 | T/C |
| 8 | 14426839 | G/A |
| 8 | 14436368 | C/A |
| 8 | 14445809 | A/G |
| 8 | 14466321 | C/A |
| 8 | 14484578 | C/G |
| 8 | 14498964 | A/G |
| 8 | 14519590 | T/C |
| 8 | 14520617 | G/A |
| 8 | 14521716 | T/C |
| 8 | 14550174 | G/A |
| 8 | 14580118 | A/G |
| 8 | 14587577 | A/G |
| 8 | 14630785 | T/C |
| 8 | 14637624 | T/C |
| 8 | 14642895 | T/G |
| 8 | 14646617 | G/A |
| 8 | 14696064 | G/A |
| 8 | 14714181 | A/G |
| 8 | 14723940 | C/T |
| 8 | 14732731 | C/T |
| 8 | 14771998 | G/A |
| 8 | 14791690 | T/C |
| 8 | 14817441 | C/G |
| 8 | 14823350 | T/A |
| 8 | 14834267 | G/T |
| 8 | 14862478 | C/T |
| 8 | 14907069 | A/G |
| 8 | 14932382 | G/A |
| 8 | 14934204 | C/T |
| 8 | 14962249 | T/C |
| 8 | 14973498 | G/A |
| 8 | 14976727 | G/A |
| 8 | 15047975 | T/G |
| 8 | 15075314 | C/T |
| 8 | 15106236 | T/A |
| 8 | 15120837 | T/C |
| 8 | 15125457 | A/G |
| 8 | 15152471 | G/A |
| 8 | 15191249 | G/A |
| 8 | 15213959 | G/A |
| 8 | 15238076 | T/C |
| 8 | 15257989 | G/C |
| 8 | 15326318 | A/G |
| 8 | 15329775 | C/G |
| 8 | 15331074 | C/G |
| 8 | 15387314 | T/G |
| 8 | 15389643 | G/T |
| 8 | 15410225 | T/C |
| 8 | 15435791 | A/T |
| 8 | 15507718 | G/T |
| 8 | 15605926 | T/C |
| 8 | 15616353 | C/A |
| 8 | 15722650 | A/T |
| 8 | 15753276 | A/G |
| 8 | 15771457 | T/C |
| 8 | 15783083 | C/G |
| 8 | 15806306 | C/T |
| 8 | 15814893 | C/A |
| 8 | 15843618 | C/T |
| 8 | 15857999 | G/A |
| 8 | 15881339 | G/A |
| 8 | 15911339 | A/T |
| 8 | 15945556 | A/G |
| 8 | 15959051 | T/C |
| 8 | 15977120 | T/C |
| 8 | 15998329 | C/T |
| 8 | 16045705 | T/C |
| 8 | 16047244 | A/G |
| 8 | 16058707 | C/G |
| 8 | 16069910 | G/A |
| 8 | 16086315 | G/A |
| 8 | 16086316 | C/T |
| 8 | 16086317 | C/T |
| 8 | 16086318 | C/T |
| 8 | 16140365 | A/G |
| 8 | 16148065 | C/T |
| 8 | 16184222 | T/C |
| 8 | 16190454 | T/C |
| 8 | 16222978 | G/A |
| 8 | 16252058 | G/T |
| 8 | 16284160 | G/A |
| 8 | 16313960 | G/A |
| 8 | 16322001 | A/G |
| 8 | 16333665 | C/T |
| 8 | 16352794 | G/A |
| 8 | 16380015 | G/A |
| 8 | 16410178 | T/G |
| 8 | 16421405 | A/T |
| 8 | 16449219 | G/A |
| 8 | 16471002 | A/C |
| 8 | 16473126 | T/C |
| 8 | 16503271 | A/C |
| 8 | 16505443 | A/G |
| 8 | 16541673 | T/C |
| 8 | 16543937 | A/G |
| 8 | 16574885 | A/T |
| 8 | 16602967 | C/A |
| 8 | 16612595 | T/A |
| 8 | 16645201 | C/T |
| 8 | 16645317 | G/C |
| 8 | 16661375 | G/A |
| 8 | 16680591 | C/T |
| 8 | 16707113 | C/T |
| 8 | 16715063 | G/T |
| 8 | 16727010 | A/G |
| 8 | 16727011 | C/G |
| 8 | 16727012 | C/G |
| 8 | 16727013 | C/G |
| 8 | 16772096 | T/C |
| 8 | 16785354 | A/G |
| 8 | 16805467 | A/G |
| 8 | 16857100 | A/G |
| 8 | 16874656 | C/T |
| 8 | 16887410 | A/G |
| 8 | 16916167 | C/T |
| 8 | 16918830 | A/G |
| 8 | 16933779 | G/A |
| 8 | 16948677 | G/A |
| 8 | 16957363 | A/C |
| 8 | 16972270 | T/C |
| 8 | 16980579 | A/T |
| 8 | 16985461 | A/G |
| 8 | 17017870 | A/G |
| 8 | 17053917 | A/C |
| 8 | 17067119 | G/A |
| 8 | 17071694 | T/C |
| 8 | 17087426 | G/A |
| 8 | 17115608 | A/T |
| 8 | 17119651 | G/A |
| 8 | 17141298 | A/G |
| 8 | 17156363 | A/G |
| 8 | 17166636 | C/T |
| 8 | 17185341 | C/A |
| 8 | 17194529 | A/T |
| 8 | 17203903 | A/C |
| 8 | 17221428 | A/G |
| 8 | 17239441 | G/A |
| 8 | 17261407 | T/C |
| 8 | 17279498 | T/G |
| 8 | 17284956 | A/C |
| 8 | 17327189 | A/G |
| 8 | 17329428 | C/A |
| 8 | 17345649 | A/G |
| 8 | 17402415 | G/A |
| 8 | 17441297 | T/C |
| 8 | 17594597 | T/C |
| 8 | 17630492 | T/A |
| 8 | 17638280 | G/A |
| 8 | 17661714 | T/G |
| 8 | 17673751 | A/C |
| 8 | 17683282 | C/A |
| 8 | 17741876 | G/T |
| 8 | 17762400 | C/A |
| 8 | 17811556 | G/A |
| 8 | 17832828 | A/T |
| 8 | 17885129 | G/A |
| 8 | 17895587 | T/C |
| 8 | 17920991 | C/T |
| 8 | 17924994 | C/T |
| 8 | 17952862 | T/C |
| 8 | 18069170 | G/A |
| 8 | 18077657 | A/T |
| 8 | 18113491 | T/C |
| 8 | 18137126 | T/C |
| 8 | 18219078 | G/A |
| 8 | 18330002 | C/T |
| 8 | 18340069 | T/C |
| 8 | 18363750 | G/A |
| 8 | 18619782 | G/T |
| 8 | 18638388 | G/A |
| 8 | 18684771 | A/G |
| 8 | 18745678 | G/A |
| 8 | 18766880 | C/T |
| 8 | 18792882 | G/T |
| 8 | 18818158 | G/T |
| 8 | 18822447 | C/A |
| 8 | 18849424 | C/T |
| 8 | 18851133 | A/G |
| 8 | 18876482 | G/A |
| 8 | 18905017 | T/C |
| 8 | 18909249 | A/G |
| 8 | 18930516 | C/T |
| 8 | 18935944 | C/A |
| 8 | 18970437 | A/T |
| 8 | 18972389 | T/C |
| 8 | 19011463 | A/T |
| 8 | 19030537 | T/C |
| 8 | 19059008 | C/T |
| 8 | 19071777 | A/G |
| 8 | 19109539 | A/G |
| 8 | 19139234 | C/G |
| 8 | 19167537 | T/A |
| 8 | 19168706 | A/G |
| 8 | 19186779 | G/A |
| 8 | 19195414 | G/A |
| 8 | 19205247 | C/A |
| 8 | 19206635 | C/T |
| 8 | 19230044 | T/C |
| 8 | 19236845 | T/A |
| 8 | 19262750 | G/T |
| 8 | 19266881 | C/T |
| 8 | 19290538 | C/A |
| 8 | 19324613 | C/T |
| 8 | 19355555 | C/T |
| 8 | 19357915 | A/T |
| 8 | 19388270 | T/A |
| 8 | 19412402 | C/T |
| 8 | 19417744 | G/A |
| 8 | 19440321 | T/C |
| 8 | 19445308 | G/C |
| 8 | 19494378 | C/A |
| 8 | 19501611 | G/T |
| 8 | 19503809 | C/T |
| 8 | 19541224 | C/G |
| 8 | 19579676 | T/A |
| 8 | 19542035 | C/T |
| 8 | 19675692 | A/C |
| 8 | 19688730 | A/G |
| 8 | 19768287 | G/A |
| 8 | 19771175 | G/A |
| 8 | 19795850 | G/A |
| 8 | 19818236 | A/G |
| 8 | 19854125 | T/C |
| 8 | 19882561 | G/A |
| 8 | 19892318 | A/T |
| 8 | 19921022 | C/T |
| 8 | 19932726 | T/C |
| 8 | 19964135 | A/G |
| 8 | 19978063 | C/T |
| 8 | 19985442 | C/T |
| 8 | 19988825 | C/G |
| 8 | 20015173 | G/A |
| 8 | 20016821 | T/G |
| 8 | 20058732 | G/A |
| 8 | 20082737 | G/C |
| 8 | 20104011 | G/A |
| 8 | 20128892 | G/A |
| 8 | 20138585 | C/T |
| 8 | 20152171 | C/T |
| 8 | 20170001 | C/A |
| 8 | 20185913 | C/T |
| 8 | 20198492 | T/C |
| 8 | 20218370 | C/A |
| 8 | 20237346 | C/T |
| 8 | 20265323 | G/C |
| 8 | 20273680 | G/A |
| 8 | 20307261 | G/A |
| 8 | 20336010 | G/A |
| 8 | 20348878 | C/T |
| 8 | 20366153 | G/A |
| 8 | 20392269 | T/A |
| 8 | 20403184 | C/T |
| 8 | 20412601 | G/A |
| 8 | 20440190 | A/G |
| 8 | 20441981 | C/T |
| 8 | 20460681 | G/A |
| 8 | 20494106 | G/A |
| 8 | 20515006 | C/T |
| 8 | 20531448 | T/C |
| 8 | 20566706 | G/C |
| 8 | 20568420 | C/T |
| 8 | 20581615 | G/A |
| 8 | 20602040 | C/T |
| 8 | 20611304 | C/G |
| 8 | 20617976 | G/A |
| 8 | 20660972 | A/C |
| 8 | 20663385 | G/C |
| 8 | 20699916 | C/T |
| 8 | 20703845 | C/T |
| 8 | 20710969 | G/A |
| 8 | 20740033 | G/T |
| 8 | 20768852 | G/A |
| 8 | 20777470 | T/G |
| 8 | 20793869 | G/T |
| 8 | 20829071 | T/C |
| 8 | 20834765 | G/A |
| 8 | 20857808 | G/A |
| 8 | 20861732 | C/T |
| 8 | 20890517 | A/C |
| 8 | 20928892 | C/T |
| 8 | 20962730 | C/T |
| 8 | 20992623 | G/A |
| 8 | 21018490 | G/A |
| 8 | 21022179 | G/C |
| 8 | 21039244 | G/T |
| 8 | 21063605 | G/A |
| 8 | 21065251 | A/G |
| 8 | 21093319 | T/G |
| 8 | 21116578 | T/A |
| 8 | 21123349 | T/A |
| 8 | 21157143 | C/A |
| 8 | 21191886 | A/T |
| 8 | 21194729 | A/G |
| 8 | 21233462 | T/C |
| 8 | 21239081 | G/A |
| 8 | 21247790 | T/C |
| 8 | 21269647 | G/A |
| 8 | 21299358 | C/T |
| 8 | 21317799 | C/T |
| 8 | 21328231 | T/G |
| 8 | 21340804 | C/A |
| 8 | 21352996 | G/A |
| 8 | 21378313 | G/A |
| 8 | 21382585 | C/A |
| 8 | 21404304 | C/A |
| 8 | 21422509 | G/A |
| 8 | 21454609 | C/G |
| 8 | 21507866 | C/G |
| 8 | 21527932 | G/C |
| 8 | 21532892 | C/T |
| 8 | 21551907 | C/T |
| 8 | 21567763 | G/A |
| 8 | 21570238 | C/T |
| 8 | 21593888 | T/C |
| 8 | 21600719 | T/A |
| 8 | 21642992 | C/T |
| 8 | 21672356 | A/T |
| 8 | 21682521 | C/A |
| 8 | 21714905 | C/T |
| 8 | 21787719 | A/C |
| 8 | 21809455 | T/C |
| 8 | 21813036 | G/A |
| 8 | 21828056 | T/C |
| 8 | 21860356 | A/T |
| 8 | 21872953 | C/T |
| 8 | 21925533 | G/A |
| 8 | 21949285 | C/A |
| 8 | 21961299 | C/A |
| 8 | 21970773 | T/G |
| 8 | 21993152 | A/G |
| 8 | 22013025 | T/A |
| 8 | 22025140 | G/A |
| 8 | 22026319 | T/C |
| 8 | 22052294 | T/C |
| 8 | 22131803 | G/A |
| 8 | 22161854 | C/A |
| 8 | 22190244 | G/A |
| 8 | 22200410 | A/G |
| 8 | 22231916 | C/A |
| 8 | 22273039 | G/T |
| 8 | 22287381 | G/C |
| 8 | 22295481 | C/T |
| 8 | 22348424 | A/G |
| 8 | 22358978 | G/A |
| 8 | 22371755 | G/A |
| 8 | 22393646 | G/A |
| 8 | 22398063 | G/A |
| 8 | 22418616 | C/T |
| 8 | 22449361 | A/C |
| 8 | 22456405 | C/A |
| 8 | 22473284 | C/T |
| 8 | 22502369 | T/C |
| 8 | 22521119 | G/A |
| 8 | 22541174 | T/C |
| 8 | 22564767 | C/A |
| 8 | 22579184 | A/G |
| 8 | 22603849 | G/A |
| 8 | 22608021 | C/T |
| 8 | 22654488 | C/A |
| 8 | 22671443 | T/G |
| 8 | 22703264 | C/T |
| 8 | 22731198 | C/T |
| 8 | 22732350 | A/G |
| 8 | 22747393 | G/A |
| 8 | 22793548 | C/T |
| 8 | 22795663 | C/G |
| 8 | 22801279 | T/A |
| 8 | 22806588 | C/T |
| 8 | 22846077 | A/G |
| 8 | 22865231 | G/A |
| 8 | 22874926 | A/G |
| 8 | 22904962 | C/T |
| 8 | 22946904 | C/T |
| 8 | 22996918 | G/A |
| 8 | 23016759 | C/T |
| 8 | 23025385 | C/T |
| 8 | 23048182 | A/G |
| 8 | 23070252 | G/A |
| 8 | 23082683 | G/A |
| 8 | 23106221 | T/C |
| 8 | 23146665 | C/T |
| 8 | 23156287 | T/C |
| 8 | 23214383 | T/C |
| 8 | 23226765 | T/A |
| 8 | 23268678 | C/T |
| 8 | 23293838 | G/A |
| 8 | 23298125 | A/T |
| 8 | 23316885 | A/T |
| 8 | 23361917 | A/G |
| 8 | 23365644 | A/C |
| 8 | 23373053 | C/A |
| 8 | 23397303 | A/G |
| 8 | 23413952 | A/G |
| 8 | 23429764 | G/T |
| 8 | 23456005 | T/A |
| 8 | 23461998 | C/G |
| 8 | 23482684 | T/A |
| 8 | 23507439 | T/C |
| 8 | 23597565 | A/G |
| 8 | 23608984 | C/A |
| 8 | 23612499 | T/C |
| 8 | 23659550 | C/T |
| 8 | 23660827 | T/C |
| 8 | 23671809 | G/A |
| 8 | 23718676 | C/A |
| 8 | 23744310 | C/T |
| 8 | 23780648 | C/T |
| 8 | 23783951 | C/T |
| 8 | 23812621 | G/T |
| 8 | 23814585 | C/T |
| 8 | 23825325 | G/T |
| 8 | 23850024 | C/T |
| 8 | 23853237 | T/C |
| 8 | 23881239 | C/T |
| 8 | 23883169 | G/T |
| 8 | 23923505 | A/G |
| 8 | 23947301 | C/T |
| 8 | 23994187 | C/T |
| 8 | 23999854 | C/T |
| 8 | 24007584 | G/A |
| 8 | 24034616 | T/C |
| 8 | 24145484 | T/A |
| 8 | 24148497 | G/C |
| 8 | 24157048 | G/A |
| 8 | 24184645 | C/G |
| 8 | 24208570 | T/G |
| 8 | 24226317 | G/A |
| 8 | 24241728 | A/G |
| 8 | 24267895 | C/T |
| 8 | 24326997 | G/A |
| 8 | 24334161 | T/C |
| 8 | 24362050 | C/T |
| 8 | 24367720 | A/G |
| 8 | 24409517 | A/T |
| 8 | 24413066 | A/G |
| 8 | 24421616 | G/A |
| 8 | 24424048 | T/C |
| 8 | 24459101 | C/T |
| 8 | 24468728 | T/C |
| 8 | 24489261 | G/A |
| 8 | 24502638 | G/A |
| 8 | 24551761 | A/G |
| 8 | 24568243 | C/T |
| 8 | 24614880 | A/T |
| 8 | 24650545 | C/T |
| 8 | 24682565 | A/G |
| 8 | 24695042 | A/G |
| 8 | 24696779 | A/G |
| 8 | 24735960 | T/G |
| 8 | 24740034 | G/T |
| 8 | 24762507 | T/C |
| 8 | 24769703 | A/C |
| 8 | 24806830 | G/A |
| 8 | 24807948 | C/T |
| 8 | 24821475 | G/A |
| 8 | 24826671 | T/G |
| 8 | 24855639 | A/G |
| 8 | 24858744 | A/C |
| 8 | 24871580 | A/T |
| 8 | 24902945 | A/G |
| 8 | 24923289 | C/T |
| 8 | 24935741 | T/C |
| 8 | 24970462 | T/C |
| 8 | 24974729 | C/T |
| 8 | 24992094 | T/C |
| 8 | 24996384 | C/T |
| 8 | 25038134 | G/T |
| 8 | 25047841 | G/A |
| 8 | 25074669 | C/T |
| 8 | 25089047 | A/G |
| 8 | 25100081 | T/G |
| 8 | 25117719 | C/T |
| 8 | 25120882 | G/A |
| 8 | 25141477 | C/T |
| 8 | 25171776 | G/C |
| 8 | 25194096 | T/C |
| 8 | 25211754 | G/C |
| 8 | 25253369 | T/G |
| 8 | 25255884 | G/A |
| 8 | 25268900 | A/T |
| 8 | 25270055 | T/A |
| 8 | 25295007 | T/A |
| 8 | 25300126 | G/T |
| 8 | 25324207 | C/T |
| 8 | 25361082 | G/A |
| 8 | 25362694 | A/G |
| 8 | 25380522 | G/A |
| 8 | 25384225 | T/G |
| 8 | 25411209 | A/G |
| 8 | 25444614 | A/G |
| 8 | 25463070 | C/A |
| 8 | 25463071 | T/C |
| 8 | 25463072 | T/C |
| 8 | 25463073 | T/C |
| 8 | 25528298 | T/C |
| 8 | 25530105 | A/G |
| 8 | 25548980 | G/A |
| 8 | 25560617 | C/T |
| 8 | 25583825 | A/G |
| 8 | 25594777 | A/T |
| 8 | 25596457 | G/A |
| 8 | 25622141 | C/T |
| 8 | 25638950 | A/G |
| 8 | 25666786 | A/T |
| 8 | 25672919 | G/T |
| 8 | 25689977 | G/T |
| 8 | 25691871 | G/A |
| 8 | 25713500 | G/A |
| 8 | 25737647 | C/A |
| 8 | 25747652 | C/A |
| 8 | 25780043 | C/G |
| 8 | 25824637 | C/G |
| 8 | 25839088 | G/A |
| 8 | 25865213 | C/G |
| 8 | 25867824 | G/T |
| 8 | 25891640 | A/C |
| 8 | 25902259 | A/T |
| 8 | 25920354 | C/G |
| 8 | 25941393 | G/A |
| 8 | 25973681 | C/T |
| 8 | 25997784 | A/T |
| 8 | 26010767 | G/A |
| 8 | 26034547 | G/A |
| 8 | 26042043 | A/C |
| 8 | 26068568 | G/C |
| 8 | 26072833 | T/C |
| 8 | 26104294 | C/G |
| 8 | 26121603 | C/T |
| 8 | 26140294 | A/G |
| 8 | 26170281 | A/G |
| 8 | 26198041 | C/T |
| 8 | 26223071 | A/G |
| 8 | 26237162 | T/C |
| 8 | 26274644 | T/G |
| 8 | 26278057 | T/C |
| 8 | 26288759 | C/T |
| 8 | 26300273 | A/C |
| 8 | 26331357 | G/A |
| 8 | 26340816 | G/A |
| 8 | 26368551 | C/G |
| 8 | 26399338 | G/A |
| 8 | 26422248 | C/A |
| 8 | 26425779 | T/A |
| 8 | 26430355 | C/T |
| 8 | 26465412 | T/G |
| 8 | 26505262 | T/G |
| 8 | 26534817 | G/A |
| 8 | 26554298 | G/A |
| 8 | 26557295 | C/G |
| 8 | 26585566 | A/G |
| 8 | 26613422 | C/A |
| 8 | 26619978 | C/T |
| 8 | 26645292 | A/G |
| 8 | 26657946 | C/T |
| 8 | 26683487 | T/C |
| 8 | 26712035 | C/T |
| 8 | 26726738 | A/G |
| 8 | 26732245 | C/T |
| 8 | 26762281 | C/A |
| 8 | 26765122 | A/C |
| 8 | 26796798 | C/G |
| 8 | 26838460 | C/T |
| 8 | 26871057 | G/A |
| 8 | 26886470 | G/A |
| 8 | 26919008 | C/T |
| 8 | 26961347 | G/A |
| 8 | 26990738 | T/C |
| 8 | 27012035 | C/G |
| 8 | 27036255 | G/A |
| 8 | 27082781 | G/A |
| 8 | 27090049 | C/T |
| 8 | 27134389 | G/A |
| 8 | 27147760 | G/A |
| 8 | 27176581 | A/G |
| 8 | 27211424 | T/C |
| 8 | 27275996 | C/T |
| 8 | 27301985 | C/T |
| 8 | 27310186 | C/T |
| 8 | 27354336 | C/T |
| 8 | 27362017 | C/T |
| 8 | 27375486 | A/G |
| 8 | 27427039 | C/T |
| 8 | 27450054 | C/T |
| 8 | 27482754 | C/T |
| 8 | 27537061 | T/A |
| 8 | 27539138 | G/A |
| 8 | 27540227 | T/G |
| 8 | 27544659 | T/G |
| 8 | 27570216 | A/G |
| 8 | 27576657 | A/C |
| 8 | 27611960 | G/T |
| 8 | 27622525 | C/T |
| 8 | 27651063 | A/G |
| 8 | 27653730 | C/T |
| 8 | 27662626 | C/T |
| 8 | 27684415 | T/C |
| 8 | 27697839 | G/T |
| 8 | 27714410 | A/G |
| 8 | 27746799 | G/T |
| 8 | 27773514 | C/T |
| 8 | 27809779 | C/T |
| 8 | 27815513 | A/G |
| 8 | 27867476 | C/T |
| 8 | 27895390 | A/G |
| 8 | 27902072 | C/T |
| 8 | 27930912 | T/G |
| 8 | 27940690 | G/A |
| 8 | 27972005 | A/G |
| 8 | 27992417 | A/G |
| 8 | 28003048 | G/T |
| 8 | 28025194 | G/A |
| 8 | 28038568 | G/T |
| 8 | 28061480 | A/T |
| 8 | 28089338 | C/T |
| 8 | 28100642 | T/C |
| 8 | 28111560 | A/G |
| 8 | 28140037 | C/A |
| 8 | 28159800 | C/T |
| 8 | 28198001 | T/C |
| 8 | 28248612 | G/C |
| 8 | 28251153 | T/C |
| 8 | 28286371 | C/G |
| 8 | 28302180 | A/G |
| 8 | 28307673 | C/A |
| 8 | 28338534 | G/T |
| 8 | 28341244 | G/A |
| 8 | 28359516 | T/A |
| 8 | 28372977 | C/G |
| 8 | 28408239 | G/A |
| 8 | 28426611 | T/C |
| 8 | 28427611 | C/T |
| 8 | 28440415 | T/A |
| 8 | 28474652 | A/G |
| 8 | 28527707 | A/C |
| 8 | 28530489 | T/C |
| 8 | 28572735 | C/T |
| 8 | 28580959 | G/A |
| 8 | 28612385 | C/T |
| 8 | 28635417 | C/T |
| 8 | 28674907 | C/T |
| 8 | 28687199 | G/A |
| 8 | 28715510 | C/T |
| 8 | 28741133 | A/G |
| 8 | 28745339 | G/A |
| 8 | 28817161 | G/A |
| 8 | 28841081 | C/A |
| 8 | 28886329 | G/C |
| 8 | 28893828 | C/G |
| 8 | 28904180 | T/G |
| 8 | 28928172 | G/A |
| 8 | 28957716 | A/G |
| 8 | 28970959 | C/G |
| 8 | 28985843 | T/C |
| 8 | 28988260 | T/C |
| 8 | 29038244 | A/G |
| 8 | 29055068 | A/G |
| 8 | 29063817 | T/C |
| 8 | 29085551 | C/T |
| 8 | 29086555 | A/G |
| 8 | 29107084 | T/C |
| 8 | 29122939 | A/G |
| 8 | 29131414 | T/G |
| 8 | 29180178 | C/A |
| 8 | 29192312 | C/T |
| 8 | 29217776 | T/C |
| 8 | 29227713 | A/G |
| 8 | 29275068 | A/G |
| 8 | 29291847 | A/G |
| 8 | 29320749 | A/T |
| 8 | 29342851 | A/G |
| 8 | 29361648 | G/C |
| 8 | 29373256 | G/A |
| 8 | 29409352 | A/G |
| 8 | 29414125 | C/A |
| 8 | 29432492 | T/A |
| 8 | 29457674 | C/T |
| 8 | 29480525 | C/A |
| 8 | 29510827 | C/G |
| 8 | 29522402 | T/G |
| 8 | 29526220 | A/G |
| 8 | 29558566 | T/C |
| 8 | 29561458 | G/A |
| 8 | 29623380 | T/A |
| 8 | 29627714 | C/T |
| 8 | 29642556 | T/C |
| 8 | 29649307 | T/C |
| 8 | 29676060 | T/C |
| 8 | 29720487 | C/T |
| 8 | 29730676 | C/A |
| 8 | 29745072 | G/A |
| 8 | 29761357 | C/A |
| 8 | 29847519 | A/G |
| 8 | 29878369 | T/C |
| 8 | 29921349 | T/A |
| 8 | 29947925 | C/A |
| 8 | 29956465 | T/C |
| 8 | 29981474 | T/C |
| 8 | 29999368 | A/G |
| 8 | 30007806 | G/A |
| 8 | 30012271 | C/G |
| 8 | 30054905 | A/G |
| 8 | 30084868 | A/G |
| 8 | 30084869 | C/G |
| 8 | 30084870 | C/G |
| 8 | 30084871 | C/G |
| 8 | 30146825 | A/C |
| 8 | 30160824 | G/A |
| 8 | 30184956 | G/A |
| 8 | 30219745 | G/A |
| 8 | 30277291 | A/G |
| 8 | 30286155 | G/A |
| 8 | 30307862 | A/C |
| 8 | 30313887 | T/C |
| 8 | 30338032 | G/A |
| 8 | 30352905 | A/G |
| 8 | 30367339 | G/A |
| 8 | 30373587 | A/C |
| 8 | 30401026 | T/G |
| 8 | 30420862 | C/T |
| 8 | 30423061 | G/A |
| 8 | 30450978 | T/G |
| 8 | 30484371 | T/C |
| 8 | 30547711 | C/T |
| 8 | 30608216 | A/G |
| 8 | 30631982 | C/T |
| 8 | 30650085 | A/G |
| 8 | 30743441 | T/C |
| 8 | 30755116 | C/T |
| 8 | 30786712 | C/T |
| 8 | 30865096 | C/T |
| 8 | 30895202 | T/C |
| 8 | 30910662 | G/A |
| 8 | 30964954 | G/A |
| 8 | 31006943 | C/G |
| 8 | 31145767 | T/C |
| 8 | 31201536 | C/G |
| 8 | 31220078 | A/C |
| 8 | 31268398 | C/G |
| 8 | 31282469 | C/A |
| 8 | 31311906 | C/G |
| 8 | 31324624 | A/G |
| 8 | 31352230 | G/A |
| 8 | 31376459 | A/G |
| 8 | 31382346 | T/C |
| 8 | 31413708 | C/T |
| 8 | 31443691 | T/C |
| 8 | 31479017 | G/A |
| 8 | 31486956 | G/A |
| 8 | 31522556 | C/G |
| 8 | 31538061 | A/C |
| 8 | 31544143 | T/C |
| 8 | 31571208 | A/G |
| 8 | 31610314 | G/A |
| 8 | 31626480 | G/A |
| 8 | 31627901 | G/A |
| 8 | 31666456 | C/G |
| 8 | 31670949 | G/A |
| 8 | 31707936 | G/A |
| 8 | 31713834 | G/A |
| 8 | 31722519 | A/T |
| 8 | 31745823 | A/G |
| 8 | 31769193 | C/G |
| 8 | 31780692 | C/T |
| 8 | 31807917 | C/T |
| 8 | 31822674 | G/A |
| 8 | 31832273 | C/T |
| 8 | 31859259 | G/A |
| 8 | 31867913 | G/A |
| 8 | 31877581 | C/G |
| 8 | 31902713 | T/C |
| 8 | 31907889 | T/G |
| 8 | 31935420 | C/T |
| 8 | 31943997 | A/G |
| 8 | 31953294 | T/A |
| 8 | 31978291 | C/T |
| 8 | 32000010 | C/T |
| 8 | 32010861 | T/C |
| 8 | 32015189 | A/T |
| 8 | 32049981 | T/C |
| 8 | 32066445 | G/A |
| 8 | 32070927 | C/T |
| 8 | 32075175 | A/G |
| 8 | 32114408 | C/G |
| 8 | 32154515 | T/C |
| 8 | 32160286 | A/G |
| 8 | 32165439 | C/T |
| 8 | 32215772 | T/C |
| 8 | 32218575 | G/A |
| 8 | 32233060 | T/A |
| 8 | 32243907 | G/A |
| 8 | 32273187 | G/A |
| 8 | 32274553 | C/G |
| 8 | 32284546 | A/G |
| 8 | 32292607 | G/A |
| 8 | 32331383 | C/T |
| 8 | 32339952 | A/G |
| 8 | 32341101 | A/T |
| 8 | 32342195 | G/A |
| 8 | 32388535 | G/A |
| 8 | 32400820 | T/C |
| 8 | 32425741 | G/A |
| 8 | 32435620 | T/A |
| 8 | 32460882 | G/A |
| 8 | 32496718 | C/G |
| 8 | 32505034 | T/C |
| 8 | 32522570 | T/G |
| 8 | 32577328 | G/T |
| 8 | 32592569 | C/T |
| 8 | 32615616 | T/C |
| 8 | 32691342 | T/C |
| 8 | 32718583 | G/A |
| 8 | 32766295 | A/G |
| 8 | 32788708 | G/T |
| 8 | 32791407 | G/A |
| 8 | 32839114 | A/G |
| 8 | 32848165 | C/A |
| 8 | 32855844 | G/A |
| 8 | 32878083 | A/G |
| 8 | 32894189 | A/G |
| 8 | 32919388 | C/T |
| 8 | 32947190 | T/C |
| 8 | 32960694 | C/A |
| 8 | 32979528 | G/A |
| 8 | 33012220 | T/C |
| 8 | 33030156 | G/C |
| 8 | 33044022 | C/T |
| 8 | 33106507 | C/T |
| 8 | 33114860 | C/T |
| 8 | 33123684 | T/C |
| 8 | 33144388 | C/T |
| 8 | 33155849 | A/G |
| 8 | 33194723 | T/C |
| 8 | 33201902 | C/T |
| 8 | 33224595 | C/T |
| 8 | 33257190 | T/C |
| 8 | 33260221 | T/C |
| 8 | 33281323 | C/T |
| 8 | 33321311 | C/G |
| 8 | 33337863 | C/T |
| 8 | 33364212 | T/A |
| 8 | 33374051 | G/A |
| 8 | 33394876 | G/A |
| 8 | 33412201 | A/G |
| 8 | 33445363 | C/T |
| 8 | 33449991 | C/T |
| 8 | 33453545 | G/C |
| 8 | 33457918 | G/A |
| 8 | 33484806 | T/C |
| 8 | 33497229 | G/T |
| 8 | 33510751 | A/G |
| 8 | 33512498 | C/A |
| 8 | 33544166 | A/G |
| 8 | 33565644 | C/T |
| 8 | 33577219 | C/A |
| 8 | 33618062 | G/A |
| 8 | 33653556 | G/A |
| 8 | 33697535 | T/G |
| 8 | 33728031 | T/C |
| 8 | 33760395 | A/G |
| 8 | 33765652 | G/A |
| 8 | 33782368 | A/G |
| 8 | 33795304 | C/A |
| 8 | 33824621 | C/T |
| 8 | 33830392 | C/T |
| 8 | 33852460 | C/T |
| 8 | 33877896 | G/A |
| 8 | 33889107 | C/T |
| 8 | 33909507 | A/T |
| 8 | 33915891 | T/C |
| 8 | 33951020 | T/C |
| 8 | 33958360 | G/C |
| 8 | 33967096 | C/T |
| 8 | 33970223 | G/A |
| 8 | 34009011 | A/T |
| 8 | 34010211 | T/C |
| 8 | 34038589 | G/A |
| 8 | 34041393 | G/A |
| 8 | 34072140 | C/A |
| 8 | 34077712 | C/G |
| 8 | 34080281 | T/C |
| 8 | 34111898 | T/C |
| 8 | 34118286 | C/T |
| 8 | 34140880 | G/C |
| 8 | 34149546 | G/A |
| 8 | 34171847 | G/T |
| 8 | 34200154 | T/C |
| 8 | 34215720 | A/G |
| 8 | 34232097 | T/C |
| 8 | 34269428 | G/A |
| 8 | 34280734 | G/C |
| 8 | 34290965 | A/G |
| 8 | 34294800 | T/G |
| 8 | 34329723 | C/T |
| 8 | 34352055 | T/G |
| 8 | 34358927 | G/A |
| 8 | 34387313 | C/T |
| 8 | 34393595 | G/A |
| 8 | 34415181 | G/A |
| 8 | 34469574 | G/A |
| 8 | 34537227 | T/C |
| 8 | 34552785 | T/A |
| 8 | 34552786 | T/A |
| 8 | 34552787 | T/A |
| 8 | 34644278 | G/T |
| 8 | 34677483 | C/T |
| 8 | 34679125 | C/T |
| 8 | 34728314 | C/A |
| 8 | 34763577 | C/A |
| 8 | 34765451 | A/G |
| 8 | 34785148 | T/C |
| 8 | 34788164 | A/G |
| 8 | 34802015 | T/C |
| 8 | 34822404 | G/A |
| 8 | 34866385 | A/T |
| 8 | 34938958 | G/C |
| 8 | 34948936 | C/G |
| 8 | 34955751 | T/C |
| 8 | 34984383 | G/A |
| 8 | 34999790 | T/G |
| 8 | 35034921 | T/C |
| 8 | 35037587 | A/T |
| 8 | 35048245 | T/A |
| 8 | 35055918 | A/G |
| 8 | 35098156 | A/T |
| 8 | 35111618 | A/G |
| 8 | 35130488 | G/T |
| 8 | 35133023 | G/T |
| 8 | 35160670 | T/C |
| 8 | 35172594 | G/A |
| 8 | 35190633 | T/C |
| 8 | 35218553 | C/A |
| 8 | 35221891 | C/T |
| 8 | 35223160 | A/G |
| 8 | 35250324 | C/T |
| 8 | 35268174 | C/A |
| 8 | 35280018 | G/C |
| 8 | 35305062 | T/C |
| 8 | 35315832 | T/C |
| 8 | 35328995 | G/A |
| 8 | 35341136 | T/A |
| 8 | 35344609 | C/A |
| 8 | 35375465 | G/A |
| 8 | 35401696 | T/C |
| 8 | 35432749 | A/T |
| 8 | 35452954 | T/C |
| 8 | 33462213 | A/T |
| 8 | 35477813 | T/A |
| 8 | 35490534 | A/G |
| 8 | 35504584 | C/G |
| 8 | 35523898 | A/C |
| 8 | 35528796 | C/A |
| 8 | 35554691 | T/C |
| 8 | 35583079 | C/T |
| 8 | 35615970 | T/C |
| 8 | 35620336 | G/C |
| 8 | 35641681 | T/C |
| 8 | 35665515 | A/G |
| 8 | 35687983 | A/C |
| 8 | 35705689 | A/G |
| 8 | 35732488 | C/T |
| 8 | 35759538 | A/T |
| 8 | 35761074 | C/G |
| 8 | 35793719 | A/G |
| 8 | 35847040 | C/T |
| 8 | 35852409 | C/T |
| 8 | 35867263 | A/G |
| 8 | 35888554 | G/A |
| 8 | 35901385 | T/G |
| 8 | 35912073 | G/A |
| 8 | 35916946 | G/C |
| 8 | 35949818 | A/G |
| 8 | 35955435 | G/A |
| 8 | 35997066 | A/G |
| 8 | 36019826 | G/T |
| 8 | 36030960 | G/A |
| 8 | 36032235 | A/G |
| 8 | 36065300 | G/C |
| 8 | 36092176 | C/T |
| 8 | 36130874 | G/A |
| 8 | 36152054 | T/A |
| 8 | 36244556 | G/A |
| 8 | 36244557 | C/A |
| 8 | 36244558 | C/A |
| 8 | 36244559 | C/A |
| 8 | 36343881 | C/T |
| 8 | 36371486 | T/C |
| 8 | 36401825 | A/G |
| 8 | 36412641 | C/A |
| 8 | 36454011 | C/T |
| 8 | 36469353 | A/G |
| 8 | 36506677 | C/T |
| 8 | 36508177 | C/T |
| 8 | 36511428 | A/C |
| 8 | 36516682 | C/A |
| 9 | 43986 | G/A |
| 9 | 58535 | G/A |
| 9 | 67207 | G/A |
| 9 | 161618 | C/T |
| 9 | 168533 | C/T |
| 9 | 250081 | G/A |
| 9 | 266605 | C/T |
| 9 | 277903 | C/T |
| 9 | 334630 | C/T |
| 9 | 374318 | T/C |
| 9 | 416616 | T/C |
| 9 | 425634 | C/T |
| 9 | 443174 | T/C |
| 9 | 451546 | G/C |
| 9 | 467245 | G/T |
| 9 | 508689 | G/A |
| 9 | 509805 | G/C |
| 9 | 525036 | G/T |
| 9 | 540569 | T/A |
| 9 | 588761 | A/C |
| 9 | 617636 | T/C |
| 9 | 644662 | G/A |
| 9 | 647067 | C/T |
| 9 | 668086 | C/T |
| 9 | 695239 | C/T |
| 9 | 720951 | G/C |
| 9 | 741248 | T/C |
| 9 | 756402 | T/A |
| 9 | 770561 | T/C |
| 9 | 781257 | T/C |
| 9 | 787588 | A/T |
| 9 | 820471 | A/T |
| 9 | 867661 | A/T |
| 9 | 872336 | A/T |
| 9 | 901348 | T/C |
| 9 | 949731 | A/G |
| 9 | 958611 | T/G |
| 9 | 960323 | T/C |
| 9 | 969734 | T/C |
| 9 | 1004296 | T/A |
| 9 | 1042851 | T/C |
| 9 | 1053145 | A/G |
| 9 | 1099813 | T/C |
| 9 | 1108902 | G/A |
| 9 | 1116673 | T/C |
| 9 | 1140961 | A/T |
| 9 | 1144734 | G/T |
| 9 | 1194012 | G/C |
| 9 | 1199169 | T/C |
| 9 | 1204281 | G/A |
| 9 | 1221646 | T/C |
| 9 | 1238333 | G/A |
| 9 | 1260510 | T/G |
| 9 | 1283333 | A/G |
| 9 | 1323816 | T/A |
| 9 | 1325116 | T/G |
| 9 | 1401851 | T/G |
| 9 | 1422588 | G/C |
| 9 | 1431317 | T/A |
| 9 | 1469854 | C/G |
| 9 | 1480044 | C/T |
| 9 | 1513344 | G/A |
| 9 | 1531893 | C/G |
| 9 | 1542573 | G/A |
| 9 | 1560155 | T/A |
| 9 | 1610197 | T/C |
| 9 | 1623351 | T/C |
| 9 | 1638499 | C/T |
| 9 | 1666960 | G/A |
| 9 | 1670136 | C/T |
| 9 | 1689838 | T/C |
| 9 | 1695937 | C/T |
| 9 | 1710321 | T/C |
| 9 | 1718363 | T/A |
| 9 | 1757000 | A/C |
| 9 | 1761360 | A/T |
| 9 | 1771385 | C/T |
| 9 | 1777497 | G/C |
| 9 | 1826578 | G/A |
| 9 | 1834052 | C/T |
| 9 | 1845860 | T/A |
| 9 | 1863385 | T/G |
| 9 | 1883226 | G/A |
| 9 | 1895353 | T/A |
| 9 | 1941543 | T/C |
| 9 | 1956543 | C/A |
| 9 | 1958670 | G/A |
| 9 | 1982381 | T/C |
| 9 | 2008378 | C/T |
| 9 | 2015034 | G/C |
| 9 | 2040089 | A/G |
| 9 | 2070721 | C/G |
| 9 | 2122713 | C/G |
| 9 | 2128159 | G/A |
| 9 | 2130636 | A/G |
| 9 | 2159865 | T/C |
| 9 | 2166252 | C/T |
| 9 | 2196822 | A/G |
| 9 | 2209574 | T/C |
| 9 | 2223270 | C/G |
| 9 | 2228934 | G/A |
| 9 | 2270341 | G/T |
| 9 | 2281261 | A/T |
| 9 | 2282267 | C/T |
| 9 | 2316726 | T/C |
| 9 | 2326577 | A/G |
| 9 | 2355010 | A/G |
| 9 | 2375631 | C/T |
| 9 | 2378467 | G/A |
| 9 | 2401922 | A/G |
| 9 | 2421168 | A/G |
| 9 | 2430240 | A/G |
| 9 | 2433821 | G/T |
| 9 | 2476162 | T/C |
| 9 | 2507463 | T/C |
| 9 | 2511249 | G/A |
| 9 | 2522908 | C/G |
| 9 | 2542855 | A/G |
| 9 | 2556607 | C/T |
| 9 | 2580903 | A/C |
| 9 | 2582433 | T/C |
| 9 | 2610055 | G/A |
| 9 | 2635455 | A/T |
| 9 | 2647306 | G/C |
| 9 | 2672137 | C/T |
| 9 | 2740020 | C/A |
| 9 | 2763514 | A/C |
| 9 | 2818123 | T/A |
| 9 | 2823503 | C/G |
| 9 | 2856122 | T/G |
| 9 | 2886778 | A/C |
| 9 | 2917920 | C/T |
| 9 | 2936227 | T/G |
| 9 | 2949318 | G/A |
| 9 | 2963152 | A/C |
| 9 | 2971791 | G/C |
| 9 | 2976805 | A/C |
| 9 | 3003486 | G/A |
| 9 | 3039569 | A/G |
| 9 | 3040680 | A/G |
| 9 | 3060114 | A/G |
| 9 | 3070771 | T/C |
| 9 | 3092524 | T/C |
| 9 | 3096055 | G/A |
| 9 | 3125059 | C/T |
| 9 | 3134863 | T/G |
| 9 | 3151740 | G/A |
| 9 | 3163765 | C/A |
| 9 | 3186670 | A/G |
| 9 | 3194287 | G/C |
| 9 | 3213867 | A/G |
| 9 | 3223013 | G/A |
| 9 | 3254871 | A/T |
| 9 | 3321556 | C/G |
| 9 | 3328231 | A/G |
| 9 | 3350290 | A/G |
| 9 | 3370706 | A/C |
| 9 | 3394295 | A/T |
| 9 | 3395527 | A/C |
| 9 | 3439847 | G/C |
| 9 | 3466058 | T/G |
| 9 | 3489620 | C/G |
| 9 | 3490824 | C/T |
| 9 | 3510666 | G/C |
| 9 | 3570001 | G/C |
| 9 | 3611667 | T/A |
| 9 | 3641354 | A/G |
| 9 | 3644749 | C/T |
| 9 | 3665087 | T/G |
| 9 | 3691771 | C/A |
| 9 | 3727983 | T/C |
| 9 | 3754532 | T/A |
| 9 | 3764003 | G/A |
| 9 | 3809261 | C/A |
| 9 | 2809262 | T/C |
| 9 | 3809263 | T/C |
| 9 | 3809264 | T/C |
| 9 | 3840650 | A/G |
| 9 | 3859311 | C/G |
| 9 | 3880904 | A/G |
| 9 | 3915777 | A/G |
| 9 | 3918165 | C/T |
| 9 | 3957874 | A/G |
| 9 | 3980852 | T/C |
| 9 | 4006709 | C/T |
| 9 | 4022109 | C/T |
| 9 | 4043347 | T/G |
| 9 | 4059543 | A/G |
| 9 | 4063778 | C/G |
| 9 | 4086196 | A/T |
| 9 | 4126014 | A/G |
| 9 | 4127630 | A/T |
| 9 | 4143019 | C/G |
| 9 | 4158657 | G/A |
| 9 | 4192980 | C/T |
| 9 | 4204275 | A/C |
| 9 | 4236089 | G/C |
| 9 | 4246873 | T/C |
| 9 | 4287861 | T/G |
| 9 | 4313555 | A/G |
| 9 | 4318031 | A/G |
| 9 | 4338140 | C/G |
| 9 | 4340774 | C/G |
| 9 | 4365053 | G/A |
| 9 | 4381511 | C/G |
| 9 | 4410045 | A/G |
| 9 | 4445080 | C/T |
| 9 | 4469087 | T/C |
| 9 | 4470743 | T/C |
| 9 | 4521864 | A/T |
| 9 | 4529970 | C/T |
| 9 | 4617035 | G/A |
| 9 | 4645373 | T/C |
| 9 | 4663953 | T/C |
| 9 | 4683673 | C/T |
| 9 | 4716349 | C/A |
| 9 | 4728343 | G/A |
| 9 | 4743085 | T/G |
| 9 | 4784220 | G/A |
| 9 | 4798126 | T/C |
| 9 | 4808297 | G/C |
| 9 | 4823663 | A/G |
| 9 | 4846640 | G/A |
| 9 | 4857233 | C/T |
| 9 | 4909437 | T/C |
| 9 | 4962466 | C/T |
| 9 | 4985542 | A/G |
| 9 | 5004511 | T/C |
| 9 | 5019500 | T/C |
| 9 | 5069368 | T/C |
| 9 | 5073486 | G/T |
| 9 | 5127303 | T/C |
| 9 | 5128348 | T/G |
| 9 | 5138745 | T/G |
| 9 | 5165106 | T/C |
| 9 | 5191086 | A/C |
| 9 | 5225306 | C/G |
| 9 | 5228534 | A/T |
| 9 | 5295755 | G/A |
| 9 | 5299233 | T/A |
| 9 | 5313393 | T/A |
| 9 | 5327314 | G/A |
| 9 | 5343893 | A/C |
| 9 | 5358306 | C/T |
| 9 | 5399776 | A/G |
| 9 | 5463020 | G/A |
| 9 | 5467312 | C/T |
| 9 | 5501454 | G/T |
| 9 | 5546070 | T/C |
| 9 | 5560367 | G/T |
| 9 | 5580064 | T/C |
| 9 | 5583887 | G/A |
| 9 | 5611745 | G/A |
| 9 | 5644154 | G/C |
| 9 | 5655794 | A/G |
| 9 | 5672925 | G/A |
| 9 | 5696716 | C/T |
| 9 | 5719890 | T/C |
| 9 | 5736005 | A/C |
| 9 | 5738897 | C/T |
| 9 | 5776483 | T/A |
| 9 | 5783124 | C/T |
| 9 | 5808579 | T/A |
| 9 | 5849159 | T/C |
| 9 | 5874050 | T/C |
| 9 | 5887051 | G/A |
| 9 | 5924311 | C/T |
| 9 | 5951756 | G/C |
| 9 | 5971876 | G/C |
| 9 | 5986744 | A/G |
| 9 | 6006044 | T/A |
| 9 | 6031744 | T/C |
| 9 | 6033191 | T/A |
| 9 | 6085509 | G/A |
| 9 | 6089012 | T/C |
| 9 | 6109289 | A/G |
| 9 | 6110876 | A/T |
| 9 | 6122834 | C/A |
| 9 | 6142306 | T/G |
| 9 | 6150405 | T/C |
| 9 | 6189211 | G/C |
| 9 | 6202589 | T/C |
| 9 | 6211255 | T/G |
| 9 | 6251068 | G/C |
| 9 | 6270104 | G/A |
| 9 | 6324635 | C/T |
| 9 | 6327090 | T/C |
| 9 | 6340501 | G/A |
| 9 | 6351465 | T/C |
| 9 | 6362078 | C/T |
| 9 | 6390302 | A/C |
| 9 | 6427375 | T/C |
| 9 | 6430049 | G/C |
| 9 | 6460914 | A/G |
| 9 | 6483674 | G/T |
| 9 | 6517940 | G/C |
| 9 | 6519464 | A/G |
| 9 | 6570018 | C/G |
| 9 | 6612665 | C/A |
| 9 | 6634532 | C/T |
| 9 | 6677247 | G/C |
| 9 | 6690194 | A/G |
| 9 | 6692550 | G/A |
| 9 | 6769986 | G/C |
| 9 | 6792671 | T/C |
| 9 | 6793903 | G/T |
| 9 | 6821584 | T/A |
| 9 | 6879346 | C/T |
| 9 | 6884365 | A/T |
| 9 | 6914086 | C/T |
| 9 | 6956997 | A/T |
| 9 | 6973899 | C/T |
| 9 | 6996038 | G/T |
| 9 | 7047476 | G/A |
| 9 | 7050713 | C/T |
| 9 | 7066396 | A/G |
| 9 | 7093627 | C/G |
| 9 | 7099212 | G/T |
| 9 | 7118725 | T/G |
| 9 | 7162131 | T/A |
| 9 | 7182622 | T/C |
| 9 | 7209696 | T/A |
| 9 | 7219261 | C/T |
| 9 | 7258382 | G/A |
| 9 | 7282854 | G/T |
| 9 | 7373738 | G/A |
| 9 | 7379638 | T/C |
| 9 | 7383044 | G/A |
| 9 | 7499152 | G/T |
| 9 | 7521030 | T/C |
| 9 | 7563658 | G/A |
| 9 | 7570941 | G/T |
| 9 | 7594807 | G/A |
| 9 | 7630344 | A/G |
| 9 | 7700129 | G/A |
| 9 | 7745162 | G/A |
| 9 | 7770964 | C/T |
| 9 | 7802146 | C/T |
| 9 | 7887515 | C/T |
| 9 | 7911729 | A/G |
| 9 | 7928078 | G/A |
| 9 | 7965384 | G/A |
| 9 | 7983894 | G/A |
| 9 | 7999831 | G/A |
| 9 | 8032321 | G/C |
| 9 | 8039416 | G/A |
| 9 | 8045552 | C/T |
| 9 | 8058058 | A/T |
| 9 | 8119239 | C/G |
| 9 | 8126707 | C/T |
| 9 | 8132280 | G/A |
| 9 | 8134699 | G/A |
| 9 | 8163066 | A/G |
| 9 | 8165678 | T/A |
| 9 | 8190898 | T/G |
| 9 | 8241442 | G/A |
| 9 | 8279944 | G/C |
| 9 | 8315518 | C/T |
| 9 | 8330819 | G/A |
| 9 | 8350208 | T/C |
| 9 | 8359903 | A/T |
| 9 | 8370031 | T/G |
| 9 | 8384109 | G/A |
| 9 | 8417464 | T/C |
| 9 | 8240624 | C/A |
| 9 | 8446284 | G/C |
| 9 | 8476507 | G/A |
| 9 | 8487975 | T/C |
| 9 | 8498671 | G/A |
| 9 | 8511034 | C/T |
| 9 | 8526257 | A/T |
| 9 | 8535859 | T/C |
| 9 | 8552316 | A/G |
| 9 | 8571742 | G/A |
| 9 | 8580589 | G/A |
| 9 | 8593434 | G/A |
| 9 | 8632415 | G/A |
| 9 | 8662463 | T/C |
| 9 | 8667253 | A/T |
| 9 | 8677382 | G/T |
| 9 | 8687374 | C/T |
| 9 | 8705434 | T/A |
| 9 | 8709356 | G/T |
| 9 | 8732209 | T/C |
| 9 | 8766202 | G/A |
| 9 | 8789458 | A/T |
| 9 | 8800413 | A/G |
| 9 | 8832578 | A/G |
| 9 | 8846322 | C/T |
| 9 | 8859760 | G/T |
| 9 | 8884726 | G/C |
| 9 | 8907156 | A/T |
| 9 | 8916575 | C/T |
| 9 | 8939338 | A/G |
| 9 | 8950058 | C/A |
| 9 | 9024292 | C/T |
| 9 | 9068058 | C/T |
| 9 | 9105961 | C/T |
| 9 | 9133579 | G/A |
| 9 | 9138436 | A/G |
| 9 | 9162039 | G/A |
| 9 | 9186149 | C/G |
| 9 | 9197657 | A/T |
| 9 | 9236009 | T/A |
| 9 | 9242242 | A/T |
| 9 | 9265706 | G/A |
| 9 | 9279089 | A/G |
| 9 | 9297432 | G/A |
| 9 | 9323389 | C/A |
| 9 | 9325326 | G/A |
| 9 | 9339415 | A/C |
| 9 | 9382473 | G/A |
| 9 | 9384611 | A/G |
| 9 | 9398819 | A/C |
| 9 | 9435919 | T/G |
| 9 | 9466571 | A/T |
| 9 | 9469412 | T/G |
| 9 | 9488715 | C/T |
| 9 | 9505302 | C/T |
| 9 | 9534161 | C/T |
| 9 | 9546461 | T/C |
| 9 | 9556839 | C/G |
| 9 | 9598822 | G/C |
| 9 | 9600794 | G/C |
| 9 | 9607877 | C/T |
| 9 | 9641410 | T/C |
| 9 | 9653598 | T/C |
| 9 | 9667954 | A/G |
| 9 | 9725574 | A/C |
| 9 | 9771585 | A/G |
| 9 | 9775983 | C/G |
| 9 | 9793697 | G/A |
| 9 | 9801287 | G/A |
| 9 | 9832416 | G/A |
| 9 | 9863825 | G/A |
| 9 | 9870005 | T/A |
| 9 | 9881038 | T/C |
| 9 | 9916537 | G/A |
| 9 | 9922976 | A/T |
| 9 | 9936791 | C/T |
| 9 | 9969996 | C/A |
| 9 | 10062164 | A/G |
| 9 | 10065993 | C/T |
| 9 | 10099020 | G/T |
| 9 | 10111900 | A/G |
| 9 | 10113121 | C/T |
| 9 | 10152868 | G/A |
| 9 | 10184140 | G/A |
| 9 | 10201865 | G/C |
| 9 | 10216944 | C/T |
| 9 | 10235873 | T/C |
| 9 | 10258447 | T/G |
| 9 | 10286419 | C/T |
| 9 | 10288089 | G/A |
| 9 | 10301851 | C/T |
| 9 | 10309828 | C/T |
| 9 | 10322017 | T/C |
| 9 | 10323823 | G/A |
| 9 | 10366524 | G/C |
| 9 | 10389514 | A/G |
| 9 | 10395188 | A/G |
| 9 | 10413778 | C/G |
| 9 | 10421460 | A/G |
| 9 | 10449247 | A/G |
| 9 | 10450871 | C/T |
| 9 | 10474793 | G/C |
| 9 | 10518574 | A/C |
| 9 | 10525754 | C/T |
| 9 | 10535589 | T/C |
| 9 | 10587085 | T/C |
| 9 | 10595806 | A/G |
| 9 | 10623285 | C/A |
| 9 | 10659026 | T/C |
| 9 | 10697966 | G/A |
| 9 | 10713420 | T/C |
| 9 | 10715884 | G/A |
| 9 | 10740178 | C/G |
| 9 | 10770487 | G/A |
| 9 | 10776158 | C/T |
| 9 | 10804135 | T/C |
| 9 | 10833653 | A/T |
| 9 | 10851937 | T/A |
| 9 | 10862786 | C/T |
| 9 | 10880966 | C/G |
| 9 | 10893817 | G/T |
| 9 | 10910406 | T/G |
| 9 | 10921821 | A/T |
| 9 | 10925609 | T/C |
| 9 | 10969856 | C/G |
| 9 | 10989648 | A/G |
| 9 | 11003179 | T/C |
| 9 | 11003180 | T/A |
| 9 | 11044009 | T/C |
| 9 | 11073833 | C/T |
| 9 | 11075077 | A/T |
| 9 | 11115735 | G/A |
| 9 | 11140509 | T/C |
| 9 | 11147324 | G/A |
| 9 | 11171801 | G/A |
| 9 | 11173863 | A/G |
| 9 | 11196373 | T/C |
| 9 | 11199988 | G/C |
| 9 | 11221848 | G/C |
| 9 | 11230675 | T/C |
| 9 | 11267145 | A/G |
| 9 | 11271544 | C/T |
| 9 | 11307918 | A/G |
| 9 | 11321865 | G/C |
| 9 | 11348031 | A/G |
| 9 | 11370136 | T/C |
| 9 | 11398183 | A/T |
| 9 | 11408126 | T/C |
| 9 | 11415622 | A/G |
| 9 | 11440068 | G/T |
| 9 | 11461587 | T/C |
| 9 | 11498412 | C/T |
| 9 | 11521379 | G/A |
| 9 | 11541255 | G/A |
| 9 | 11557517 | C/G |
| 9 | 11572558 | C/T |
| 9 | 11586750 | T/A |
| 9 | 11602129 | T/C |
| 9 | 11614254 | A/G |
| 9 | 11617052 | T/C |
| 9 | 11664594 | C/T |
| 9 | 11679384 | A/G |
| 9 | 11696053 | T/C |
| 9 | 11712338 | A/G |
| 9 | 11727568 | C/T |
| 9 | 11734470 | G/T |
| 9 | 11765880 | T/C |
| 9 | 11803951 | T/G |
| 9 | 11834727 | G/A |
| 9 | 11896652 | C/T |
| 9 | 11989308 | C/T |
| 9 | 12014322 | G/A |
| 9 | 12045869 | G/C |
| 9 | 12066084 | T/A |
| 9 | 12089794 | T/G |
| 9 | 12091354 | T/C |
| 9 | 12127079 | C/T |
| 9 | 12136220 | A/G |
| 9 | 12160996 | C/T |
| 9 | 12193396 | G/A |
| 9 | 12209484 | G/A |
| 9 | 12215272 | A/G |
| 9 | 12237936 | A/G |
| 9 | 12252359 | G/C |
| 9 | 12275043 | G/A |
| 9 | 12312275 | A/C |
| 9 | 12314306 | T/C |
| 9 | 12353847 | C/T |
| 9 | 12376457 | G/A |
| 9 | 12406245 | C/G |
| 9 | 12408416 | T/C |
| 9 | 12445735 | T/A |
| 9 | 12453413 | C/T |
| 9 | 12479324 | T/C |
| 9 | 12483140 | T/G |
| 9 | 12485001 | G/C |
| 9 | 12516836 | A/T |
| 9 | 12539441 | C/G |
| 9 | 12548196 | C/T |
| 9 | 12579410 | C/T |
| 9 | 12591765 | T/C |
| 9 | 12624185 | G/A |
| 9 | 12629168 | C/G |
| 9 | 12656344 | A/C |
| 9 | 12673475 | T/C |
| 9 | 12697151 | A/C |
| 9 | 12701767 | T/G |
| 9 | 12787295 | C/A |
| 9 | 12809375 | C/T |
| 9 | 12818127 | C/T |
| 9 | 12834770 | A/G |
| 9 | 12840171 | T/A |
| 9 | 12869427 | A/G |
| 9 | 12887518 | A/G |
| 9 | 12895669 | G/A |
| 9 | 12901726 | G/A |
| 9 | 12946296 | A/T |
| 9 | 12949990 | A/C |
| 9 | 13004011 | G/A |
| 9 | 13047537 | A/G |
| 9 | 13050166 | G/A |
| 9 | 13084778 | G/A |
| 9 | 13136004 | T/C |
| 9 | 13139783 | C/A |
| 9 | 13156389 | T/C |
| 9 | 13157433 | T/C |
| 9 | 13179252 | T/C |
| 9 | 13188728 | G/T |
| 9 | 13200080 | C/T |
| 9 | 13236751 | A/G |
| 9 | 13258399 | A/G |
| 9 | 13279264 | A/G |
| 9 | 13282450 | G/A |
| 9 | 13323935 | A/C |
| 9 | 13344138 | A/G |
| 9 | 13351933 | T/C |
| 9 | 13377974 | T/G |
| 9 | 13403880 | C/G |
| 9 | 13412235 | C/T |
| 9 | 13421582 | A/G |
| 9 | 13448708 | G/A |
| 9 | 13477253 | T/C |
| 9 | 13502275 | G/A |
| 9 | 13519538 | C/T |
| 9 | 13532131 | A/C |
| 9 | 13560010 | T/G |
| 9 | 13561116 | T/C |
| 9 | 13593484 | A/G |
| 9 | 13609923 | A/G |
| 9 | 13621206 | T/C |
| 9 | 13647416 | A/G |
| 9 | 13663000 | A/G |
| 9 | 13671730 | T/A |
| 9 | 13698111 | C/T |
| 9 | 13735725 | A/G |
| 9 | 13741536 | T/C |
| 9 | 13744487 | A/C |
| 9 | 13780115 | C/A |
| 9 | 13829909 | A/G |
| 9 | 13854803 | C/T |
| 9 | 13924182 | C/T |
| 9 | 13927367 | G/A |
| 9 | 13950652 | G/A |
| 9 | 13960447 | A/G |
| 9 | 13981138 | A/G |
| 9 | 14023001 | G/T |
| 9 | 14043745 | T/C |
| 9 | 14071179 | G/T |
| 9 | 14098470 | G/A |
| 9 | 14114782 | C/T |
| 9 | 14155835 | G/A |
| 9 | 14165786 | T/C |
| 9 | 14189817 | G/A |
| 9 | 14216099 | T/C |
| 9 | 14227135 | G/A |
| 9 | 14247424 | C/T |
| 9 | 14250346 | T/C |
| 9 | 14276764 | G/A |
| 9 | 14309702 | C/T |
| 9 | 14318067 | A/G |
| 9 | 14350555 | C/T |
| 9 | 14368607 | G/C |
| 9 | 14385539 | A/G |
| 9 | 14399877 | G/T |
| 9 | 14402212 | C/T |
| 9 | 14447649 | G/A |
| 9 | 14453766 | C/T |
| 9 | 14463010 | A/T |
| 9 | 14501530 | A/T |
| 9 | 14516421 | G/A |
| 9 | 14521974 | G/A |
| 9 | 14542075 | C/T |
| 9 | 14561309 | A/G |
| 9 | 14586098 | G/A |
| 9 | 14647228 | T/C |
| 9 | 14705806 | G/A |
| 9 | 14743720 | T/C |
| 9 | 14782605 | A/G |
| 9 | 14795961 | A/T |
| 9 | 14836260 | T/C |
| 9 | 14869599 | T/C |
| 9 | 14871472 | C/A |
| 9 | 14880959 | C/A |
| 9 | 14899570 | G/A |
| 9 | 14951907 | C/G |
| 9 | 14969509 | A/C |
| 9 | 15007267 | T/C |
| 9 | 15033788 | T/G |
| 9 | 15033789 | A/G |
| 9 | 15065351 | C/T |
| 9 | 15081404 | A/G |
| 9 | 15107215 | G/A |
| 9 | 15111917 | G/T |
| 9 | 15138354 | G/C |
| 9 | 15160252 | A/G |
| 9 | 15161307 | T/A |
| 9 | 15202493 | C/T |
| 9 | 15211882 | G/A |
| 9 | 15221523 | T/C |
| 9 | 15251034 | T/C |
| 9 | 15270723 | G/T |
| 9 | 15319982 | A/G |
| 9 | 15343542 | G/A |
| 9 | 15348258 | C/G |
| 9 | 15407635 | G/C |
| 9 | 15415013 | T/C |
| 9 | 15421510 | C/T |
| 9 | 15473848 | T/C |
| 9 | 15480347 | G/A |
| 9 | 15557120 | T/G |
| 9 | 15566901 | A/C |
| 9 | 15576958 | G/A |
| 9 | 15579441 | T/C |
| 9 | 15620752 | G/T |
| 9 | 15656982 | C/T |
| 9 | 15659680 | G/A |
| 9 | 15674222 | T/C |
| 9 | 15707298 | G/A |
| 9 | 15722777 | G/A |
| 9 | 15740851 | G/A |
| 9 | 15761473 | A/C |
| 9 | 15777981 | G/A |
| 9 | 15806460 | C/G |
| 9 | 15833435 | G/A |
| 9 | 15835564 | A/C |
| 9 | 15840054 | T/G |
| 9 | 15868613 | T/A |
| 9 | 15870460 | T/C |
| 9 | 15877184 | G/A |
| 9 | 15911092 | G/A |
| 9 | 15912114 | G/T |
| 9 | 15947189 | C/T |
| 9 | 15963813 | A/C |
| 9 | 15971852 | G/A |
| 9 | 15993128 | C/T |
| 9 | 16042853 | T/C |
| 9 | 16078120 | T/C |
| 9 | 16079496 | A/G |
| 9 | 16083138 | C/G |
| 9 | 16104890 | A/G |
| 9 | 16114977 | T/A |
| 9 | 16123410 | C/T |
| 9 | 16145933 | C/T |
| 9 | 16193612 | C/T |
| 9 | 16224339 | A/G |
| 9 | 16248555 | C/T |
| 9 | 16289467 | C/G |
| 9 | 16296537 | C/T |
| 9 | 16304490 | G/T |
| 9 | 16320739 | G/C |
| 9 | 16337107 | G/T |
| 9 | 16362830 | G/A |
| 9 | 16363950 | G/A |
| 9 | 16382136 | C/T |
| 9 | 16420066 | C/A |
| 9 | 16440674 | G/T |
| 9 | 16442117 | C/T |
| 9 | 16474323 | G/A |
| 9 | 16477291 | G/A |
| 9 | 16522274 | A/G |
| 9 | 16530624 | T/A |
| 9 | 16563701 | A/T |
| 9 | 16574855 | T/C |
| 9 | 16590896 | T/G |
| 9 | 16643846 | C/T |
| 9 | 16650568 | T/G |
| 9 | 16740169 | G/A |
| 9 | 16764808 | A/G |
| 9 | 16824637 | A/G |
| 9 | 16843537 | A/G |
| 9 | 16854146 | A/G |
| 9 | 16879891 | T/C |
| 9 | 16885157 | C/T |
| 9 | 16913206 | G/A |
| 9 | 16920010 | C/T |
| 9 | 16939678 | T/C |
| 9 | 16950374 | C/T |
| 9 | 16963946 | T/A |
| 9 | 17000437 | G/A |
| 9 | 17004961 | T/C |
| 9 | 17029491 | T/C |
| 9 | 17079512 | A/G |
| 9 | 17088358 | C/T |
| 9 | 17110643 | T/C |
| 9 | 17126122 | G/A |
| 9 | 17153635 | A/G |
| 9 | 17166264 | T/C |
| 9 | 17178155 | A/G |
| 9 | 17193200 | A/C |
| 9 | 17231751 | T/G |
| 9 | 17237071 | T/C |
| 9 | 17264627 | C/T |
| 9 | 17276445 | A/C |
| 9 | 17285550 | G/A |
| 9 | 17326909 | C/A |
| 9 | 17338538 | G/A |
| 9 | 17349041 | T/G |
| 9 | 17357477 | A/T |
| 9 | 17370121 | T/C |
| 9 | 17408055 | G/A |
| 9 | 17414185 | A/G |
| 9 | 17458282 | C/T |
| 9 | 17461359 | G/A |
| 9 | 17487915 | C/T |
| 9 | 17514612 | T/A |
| 9 | 17530434 | T/G |
| 9 | 17537237 | A/C |
| 9 | 17562558 | G/T |
| 9 | 17587407 | T/G |
| 9 | 17620099 | C/T |
| 9 | 17641845 | G/C |
| 9 | 17675417 | A/T |
| 9 | 17683612 | T/G |
| 9 | 17722981 | T/C |
| 9 | 17730340 | T/C |
| 9 | 17776408 | A/C |
| 9 | 17793860 | T/A |
| 9 | 17816789 | G/T |
| 9 | 17831314 | A/T |
| 9 | 17859945 | A/T |
| 9 | 17943535 | C/A |
| 9 | 18029543 | T/C |
| 9 | 18032422 | C/T |
| 9 | 18033480 | G/A |
| 9 | 18089992 | A/G |
| 9 | 18094773 | A/G |
| 9 | 18107002 | C/G |
| 9 | 18124860 | A/G |
| 9 | 18163894 | A/T |
| 9 | 18198830 | A/T |
| 9 | 18218753 | G/A |
| 9 | 18267620 | A/G |
| 9 | 18311242 | C/T |
| 9 | 18359657 | T/C |
| 9 | 18378281 | G/C |
| 9 | 18393190 | C/T |
| 9 | 18425968 | A/G |
| 9 | 18427130 | T/G |
| 9 | 18450840 | A/G |
| 9 | 18462784 | C/T |
| 9 | 18487075 | C/A |
| 9 | 18519709 | G/A |
| 9 | 18530134 | C/T |
| 9 | 18561760 | C/T |
| 9 | 18572535 | G/A |
| 9 | 18620835 | C/T |
| 9 | 18629065 | C/T |
| 9 | 18635242 | C/T |
| 9 | 18637332 | T/G |
| 9 | 18664318 | G/A |
| 9 | 18686061 | A/G |
| 9 | 18691485 | T/C |
| 9 | 18720026 | A/C |
| 9 | 18747135 | G/A |
| 9 | 18764705 | T/G |
| 9 | 18776991 | G/T |
| 9 | 18799535 | G/A |
| 9 | 18814658 | A/G |
| 9 | 18817459 | A/C |
| 9 | 18849218 | T/C |
| 9 | 18856895 | G/A |
| 9 | 18881745 | T/C |
| 9 | 18884821 | A/G |
| 9 | 18900331 | T/C |
| 9 | 18930492 | C/G |
| 9 | 18955813 | C/T |
| 9 | 19005299 | T/C |
| 9 | 19025361 | T/C |
| 9 | 19039162 | T/A |
| 9 | 19055054 | A/G |
| 9 | 19080631 | G/A |
| 9 | 19117857 | C/G |
| 9 | 19123561 | G/A |
| 9 | 19140376 | T/A |
| 9 | 19243692 | G/A |
| 9 | 19271782 | C/T |
| 9 | 19292159 | C/G |
| 9 | 19320612 | T/C |
| 9 | 19371689 | C/G |
| 9 | 19405511 | A/C |
| 9 | 19410196 | G/A |
| 9 | 19442226 | C/T |
| 9 | 19596641 | T/A |
| 9 | 19642451 | C/G |
| 9 | 19703541 | A/G |
| 9 | 19706213 | A/C |
| 9 | 19723356 | G/A |
| 9 | 19725330 | A/G |
| 9 | 19823342 | A/G |
| 9 | 19870309 | C/A |
| 9 | 19872019 | T/C |
| 9 | 19912290 | A/G |
| 9 | 19926736 | G/A |
| 9 | 19948302 | A/G |
| 9 | 19965915 | T/C |
| 9 | 19996094 | C/T |
| 9 | 20002244 | A/G |
| 9 | 20020727 | A/G |
| 9 | 20046160 | G/A |
| 9 | 20047762 | C/T |
| 9 | 20071682 | T/C |
| 9 | 20078343 | G/A |
| 9 | 20110451 | A/G |
| 9 | 20138920 | T/C |
| 9 | 20170834 | T/C |
| 9 | 20176185 | C/T |
| 9 | 20212059 | G/T |
| 9 | 20240214 | T/C |
| 9 | 20273322 | A/G |
| 9 | 20285570 | G/A |
| 9 | 20324714 | C/T |
| 9 | 20337421 | A/C |
| 9 | 20349941 | C/G |
| 9 | 20424558 | C/T |
| 9 | 20506811 | G/A |
| 9 | 20556596 | T/C |
| 9 | 20623734 | T/C |
| 9 | 20630483 | A/G |
| 9 | 20673536 | C/T |
| 9 | 20678515 | A/G |
| 9 | 20700008 | C/T |
| 9 | 20722349 | T/C |
| 9 | 20730852 | A/G |
| 9 | 20740827 | T/C |
| 9 | 20789951 | C/T |
| 9 | 20802996 | A/T |
| 9 | 20813775 | A/C |
| 9 | 20836927 | T/C |
| 9 | 20849088 | G/A |
| 9 | 20869682 | A/C |
| 9 | 20875301 | T/C |
| 9 | 20905870 | G/T |
| 9 | 20912874 | T/A |
| 9 | 20916760 | A/G |
| 9 | 20967774 | C/A |
| 9 | 21094729 | G/A |
| 9 | 21172277 | A/C |
| 9 | 21174615 | C/T |
| 9 | 21180720 | G/A |
| 9 | 21193142 | G/A |
| 9 | 21216803 | A/C |
| 9 | 21259740 | A/G |
| 9 | 21264453 | C/G |
| 9 | 21299521 | T/G |
| 9 | 21301743 | A/T |
| 9 | 21323840 | A/G |
| 9 | 21337276 | C/A |
| 9 | 21360877 | A/C |
| 9 | 21367860 | T/C |
| 9 | 21407507 | T/A |
| 9 | 21440483 | A/C |
| 9 | 21444248 | A/C |
| 9 | 21467809 | T/C |
| 9 | 21469066 | C/T |
| 9 | 21487428 | T/C |
| 9 | 21489918 | T/A |
| 9 | 21510672 | C/G |
| 9 | 21546847 | C/T |
| 9 | 21574889 | G/A |
| 9 | 21619865 | A/C |
| 9 | 21620971 | A/G |
| 9 | 21652973 | T/G |
| 9 | 21659392 | G/T |
| 9 | 21663578 | T/G |
| 9 | 21665670 | T/C |
| 9 | 21690701 | G/C |
| 9 | 21749049 | T/G |
| 9 | 21773440 | C/T |
| 9 | 21776157 | G/A |
| 9 | 21791082 | A/T |
| 9 | 21795529 | C/T |
| 9 | 21817265 | A/G |
| 9 | 21823985 | C/T |
| 9 | 21863525 | A/G |
| 9 | 21875524 | A/G |
| 9 | 21878118 | A/C |
| 9 | 21918442 | T/C |
| 9 | 21921630 | C/T |
| 9 | 21958216 | G/A |
| 9 | 21986319 | A/T |
| 9 | 21988046 | C/T |
| 9 | 22013514 | C/T |
| 9 | 22044319 | T/C |
| 9 | 22071086 | C/T |
| 9 | 22086433 | G/A |
| 9 | 22092066 | G/A |
| 9 | 22122062 | G/A |
| 9 | 22160413 | G/A |
| 9 | 22173946 | C/T |
| 9 | 22218421 | A/T |
| 9 | 22228699 | G/A |
| 9 | 22230947 | A/G |
| 9 | 22256652 | C/T |
| 9 | 22281187 | C/T |
| 9 | 22318810 | G/T |
| 9 | 22320148 | G/A |
| 9 | 22324510 | G/A |
| 9 | 22355776 | G/A |
| 9 | 22385591 | G/T |
| 9 | 22407493 | T/C |
| 9 | 22418085 | C/T |
| 9 | 22421065 | C/G |
| 9 | 22451276 | C/T |
| 9 | 22457267 | A/G |
| 9 | 22470252 | C/T |
| 9 | 22501758 | T/C |
| 9 | 22530679 | C/T |
| 9 | 22552559 | T/C |
| 9 | 22562281 | T/C |
| 9 | 22581600 | G/A |
| 9 | 22590529 | A/C |
| 9 | 22592120 | G/A |
| 9 | 22622788 | A/G |
| 9 | 22639351 | G/A |
| 9 | 22661531 | G/A |
| 9 | 22676320 | A/G |
| 9 | 22684800 | A/T |
| 9 | 22708187 | A/T |
| 9 | 22712891 | A/G |
| 9 | 22718682 | A/G |
| 9 | 22754228 | G/A |
| 9 | 22772535 | G/T |
| 9 | 22800799 | A/C |
| 9 | 22810718 | T/A |
| 9 | 22900618 | A/G |
| 9 | 22905473 | T/C |
| 9 | 22937846 | G/A |
| 9 | 22951232 | G/A |
| 9 | 22957898 | C/T |
| 9 | 22985287 | C/T |
| 9 | 23016531 | T/G |
| 9 | 23017931 | G/T |
| 9 | 23049995 | G/A |
| 9 | 23057322 | A/T |
| 9 | 23095174 | G/A |
| 9 | 23100774 | C/A |
| 9 | 23112554 | C/T |
| 9 | 23145358 | T/C |
| 9 | 23172235 | A/T |
| 9 | 23201893 | G/A |
| 9 | 23209322 | G/A |
| 9 | 23242726 | A/G |
| 9 | 23243991 | T/G |
| 9 | 23259799 | A/G |
| 9 | 23271248 | C/T |
| 9 | 23295632 | G/A |
| 9 | 23310262 | G/T |
| 9 | 23344728 | C/T |
| 9 | 23359269 | G/A |
| 9 | 23392356 | G/A |
| 9 | 23397758 | G/A |
| 9 | 23400997 | A/T |
| 9 | 23431477 | G/A |
| 9 | 23433194 | G/T |
| 9 | 23530575 | T/C |
| 9 | 23572618 | T/G |
| 9 | 23619436 | G/A |
| 9 | 23652107 | A/T |
| 9 | 23666499 | A/T |
| 9 | 23717161 | G/C |
| 9 | 23767774 | G/A |
| 9 | 23776990 | T/C |
| 9 | 23791002 | A/T |
| 9 | 23795968 | A/G |
| 9 | 23827391 | G/A |
| 9 | 23875287 | T/G |
| 9 | 23880109 | G/C |
| 9 | 23904126 | C/T |
| 9 | 23911951 | T/C |
| 9 | 23945882 | A/G |
| 9 | 23986492 | G/A |
| 9 | 24042675 | T/A |
| 9 | 24045175 | C/G |
| 9 | 24070450 | G/A |
| 9 | 24073841 | C/A |
| 9 | 24102071 | G/A |
| 9 | 24120889 | C/T |
| 9 | 24159801 | A/C |
| 9 | 24201827 | G/A |
| 9 | 24215437 | C/T |
| 9 | 24227269 | A/T |
| 9 | 24256128 | T/A |
| 9 | 24262153 | C/G |
| 9 | 24270880 | G/C |
| 9 | 24301552 | T/C |
| 9 | 24331077 | C/T |
| 9 | 24345675 | C/G |
| 9 | 24360550 | C/G |
| 9 | 24375729 | T/C |
| 9 | 24418508 | G/A |
| 9 | 24471612 | A/G |
| 9 | 24481640 | A/G |
| 9 | 24510710 | A/G |
| 9 | 24542000 | C/T |
| 9 | 24548592 | C/T |
| 9 | 24588988 | A/C |
| 9 | 24591096 | A/G |
| 9 | 24620069 | A/G |
| 9 | 24642178 | A/G |
| 9 | 24680852 | T/C |
| 9 | 24688108 | C/G |
| 9 | 24692585 | C/G |
| 9 | 24714125 | T/C |
| 9 | 24723055 | A/G |
| 9 | 24747570 | G/C |
| 9 | 24751385 | C/T |
| 9 | 24764011 | T/C |
| 9 | 24796291 | C/T |
| 9 | 24799205 | G/A |
| 9 | 24827294 | G/A |
| 9 | 24830096 | G/A |
| 9 | 24865622 | T/A |
| 9 | 24877294 | G/C |
| 9 | 24913312 | G/A |
| 9 | 24949393 | C/T |
| 9 | 24957864 | C/T |
| 9 | 24969302 | G/A |
| 9 | 24982497 | G/A |
| 9 | 24992911 | G/A |
| 9 | 24997588 | C/T |
| 9 | 25042450 | A/C |
| 9 | 25049458 | A/G |
| 9 | 25060076 | A/T |
| 9 | 25100682 | C/T |
| 9 | 25102400 | C/T |
| 9 | 25122997 | G/A |
| 9 | 25126947 | C/T |
| 9 | 25162910 | T/A |
| 9 | 25180804 | C/T |
| 9 | 25212958 | C/T |
| 9 | 25256939 | C/T |
| 9 | 25266573 | C/T |
| 9 | 25269876 | C/T |
| 9 | 25302561 | C/G |
| 9 | 25355353 | A/T |
| 9 | 25382707 | A/T |
| 9 | 25420456 | C/A |
| 9 | 25441685 | G/T |
| 9 | 25449274 | T/A |
| 9 | 25496815 | T/A |
| 9 | 25506618 | C/T |
| 9 | 25512156 | G/A |
| 9 | 25551167 | T/A |
| 9 | 25556436 | A/G |
| 9 | 25581699 | G/A |
| 9 | 25603407 | G/A |
| 9 | 25617956 | C/T |
| 9 | 25628028 | C/T |
| 9 | 25676632 | G/C |
| 9 | 25684324 | T/G |
| 9 | 25712475 | G/A |
| 9 | 25718642 | C/T |
| 9 | 25765205 | T/A |
| 9 | 25773648 | T/A |
| 9 | 25777856 | G/A |
| 9 | 25825209 | G/A |
| 9 | 25826501 | C/T |
| 9 | 25835560 | A/G |
| 9 | 25850697 | T/C |
| 9 | 25873631 | G/C |
| 9 | 25917578 | C/A |
| 9 | 25929090 | A/G |
| 9 | 26022214 | C/T |
| 9 | 26079099 | G/A |
| 9 | 26112925 | G/A |
| 9 | 26212071 | C/T |
| 9 | 26239733 | C/T |
| 9 | 26276110 | C/G |
| 9 | 26300267 | G/A |
| 9 | 26337475 | G/A |
| 9 | 26356324 | T/C |
| 9 | 26388854 | C/T |
| 9 | 26454532 | G/A |
| 9 | 26462024 | G/A |
| 9 | 26481700 | C/T |
| 9 | 26491846 | T/C |
| 9 | 26520716 | C/T |
| 9 | 26596720 | C/T |
| 9 | 26672496 | C/T |
| 9 | 26710947 | A/G |
| 9 | 26726683 | A/G |
| 9 | 26734842 | A/G |
| 9 | 26871690 | C/T |
| 9 | 26884419 | G/C |
| 9 | 26953863 | C/T |
| 9 | 26989211 | C/T |
| 9 | 27011194 | A/T |
| 9 | 27022364 | C/A |
| 9 | 27034956 | A/G |
| 9 | 27107307 | A/G |
| 9 | 27138798 | C/T |
| 9 | 27169677 | C/A |
| 9 | 27186248 | G/A |
| 9 | 27198675 | T/A |
| 9 | 27234079 | T/C |
| 9 | 27268240 | T/C |
| 9 | 27279786 | G/C |
| 9 | 27299025 | A/G |
| 9 | 27319003 | G/A |
| 9 | 27338366 | C/G |
| 9 | 27354708 | G/A |
| 9 | 27380925 | G/A |
| 9 | 27410081 | T/C |
| 9 | 27429230 | A/G |
| 9 | 27471115 | G/A |
| 9 | 27494770 | T/C |
| 9 | 27530750 | T/C |
| 9 | 27536061 | T/C |
| 9 | 24542057 | T/A |
| 9 | 27555680 | C/T |
| 9 | 27581063 | C/T |
| 9 | 27617087 | T/G |
| 9 | 27652287 | T/C |
| 9 | 27657221 | C/G |
| 9 | 27664916 | C/A |
| 9 | 27666020 | G/A |
| 9 | 27711303 | T/C |
| 9 | 27734313 | C/T |
| 9 | 27736335 | C/T |
| 9 | 27754776 | G/T |
| 9 | 27791108 | G/C |
| 9 | 27795452 | T/G |
| 9 | 27822622 | C/G |
| 9 | 27840023 | A/T |
| 9 | 27885111 | C/T |
| 9 | 27908810 | A/G |
| 9 | 27928627 | A/T |
| 9 | 27930774 | T/G |
| 9 | 27962180 | A/T |
| 9 | 27966663 | G/A |
| 9 | 27996753 | T/C |
| 9 | 28047561 | C/T |
| 9 | 28061799 | G/A |
| 9 | 28113175 | A/T |
| 9 | 28159593 | C/T |
| 9 | 28175860 | C/T |
| 9 | 28186206 | C/A |
| 9 | 28258224 | A/G |
| 9 | 28260927 | T/A |
| 9 | 28342413 | G/A |
| 9 | 28342414 | C/T |
| 9 | 28342415 | C/T |
| 9 | 28342416 | C/T |
| 9 | 28404251 | T/C |
| 9 | 28436167 | A/T |
| 9 | 28439389 | T/C |
| 9 | 28439390 | G/C |
| 9 | 28439391 | G/C |
| 9 | 28439392 | G/C |
| 9 | 28552211 | A/G |
| 9 | 28558207 | G/A |
| 9 | 28567413 | T/C |
| 9 | 28569606 | C/T |
| 9 | 28593487 | T/C |
| 9 | 28640231 | C/T |
| 9 | 28643256 | A/T |
| 9 | 28650436 | C/T |
| 9 | 28677117 | C/T |
| 9 | 28686065 | C/T |
| 9 | 28722560 | C/A |
| 9 | 28728810 | G/A |
| 9 | 28759290 | A/T |
| 9 | 28824834 | C/A |
| 9 | 28826972 | T/G |
| 9 | 28830981 | A/C |
| 9 | 28883539 | C/T |
| 9 | 28897994 | C/T |
| 9 | 28907420 | G/A |
| 9 | 28951638 | C/A |
| 9 | 28966947 | T/C |
| 9 | 29017071 | G/A |
| 9 | 29047192 | G/A |
| 9 | 29095640 | C/G |
| 9 | 29122571 | A/C |
| 9 | 29126077 | A/G |
| 9 | 29158447 | T/G |
| 9 | 29160362 | C/T |
| 9 | 29191223 | A/G |
| 9 | 29224444 | A/C |
| 9 | 29251007 | T/G |
| 9 | 29277980 | G/A |
| 9 | 29289406 | G/A |
| 9 | 29325512 | T/C |
| 9 | 29362580 | G/A |
| 9 | 29368485 | T/C |
| 9 | 29392466 | A/T |
| 9 | 29401545 | T/C |
| 9 | 29455806 | G/A |
| 9 | 29464126 | C/T |
| 9 | 29533589 | T/C |
| 9 | 29534914 | G/A |
| 9 | 29601430 | C/T |
| 9 | 29606942 | A/G |
| 9 | 29625215 | A/G |
| 9 | 29666473 | G/A |
| 9 | 29684565 | C/T |
| 9 | 29689633 | C/T |
| 9 | 29749628 | G/A |
| 9 | 29760235 | C/T |
| 9 | 29799175 | C/T |
| 9 | 29829132 | T/C |
| 9 | 29851054 | C/T |
| 9 | 29866283 | G/A |
| 9 | 29884392 | G/A |
| 9 | 29922239 | G/A |
| 9 | 29927322 | C/T |
| 9 | 29941186 | C/G |
| 9 | 29949179 | T/C |
| 9 | 29974374 | T/A |
| 9 | 30008053 | A/C |
| 9 | 30018322 | C/G |
| 9 | 30036026 | C/T |
| 9 | 30061659 | G/A |
| 9 | 30087287 | C/T |
| 9 | 30094639 | T/C |
| 9 | 30111801 | A/G |
| 9 | 30128624 | A/G |
| 9 | 30144568 | G/C |
| 9 | 30151423 | G/A |
| 9 | 30160513 | A/C |
| 9 | 30191285 | G/T |
| 9 | 30211120 | G/T |
| 9 | 30264275 | T/C |
| 9 | 30270874 | G/A |
| 9 | 30274093 | A/G |
| 9 | 30326216 | C/T |
| 9 | 30375891 | C/T |
| 9 | 30386474 | C/A |
| 9 | 30441232 | G/A |
| 9 | 30508715 | G/T |
| 9 | 30534366 | C/T |
| 9 | 30561073 | C/T |
| 9 | 30565222 | T/C |
| 9 | 30598997 | A/C |
| 9 | 30637093 | C/T |
| 9 | 30692258 | C/T |
| 9 | 30760914 | A/G |
| 9 | 30795726 | C/T |
| 9 | 30837191 | C/T |
| 9 | 30860943 | G/A |
| 9 | 30889368 | G/C |
| 9 | 30891314 | C/T |
| 9 | 30913053 | A/C |
| 9 | 30922603 | C/A |
| 9 | 30931891 | C/T |
| 9 | 30932974 | A/G |
| 9 | 30961934 | C/T |
| 9 | 30963379 | G/A |
| 9 | 30991947 | C/T |
| 9 | 31043058 | T/G |
| 9 | 31051477 | A/G |
| 9 | 31087941 | C/T |
| 9 | 31132730 | G/C |
| 9 | 31154903 | C/T |
| 9 | 31182317 | T/A |
| 9 | 31251190 | G/A |
| 9 | 31276477 | A/C |
| 9 | 31284293 | G/A |
| 9 | 31297430 | C/T |
| 9 | 31301935 | C/T |
| 9 | 31336258 | G/A |
| 9 | 31374150 | G/A |
| 9 | 31383339 | C/T |
| 9 | 31384865 | C/G |
| 9 | 31425390 | T/A |
| 9 | 31439340 | T/C |
| 9 | 31446110 | G/T |
| 9 | 31479297 | G/A |
| 9 | 31505147 | G/C |
| 9 | 31526103 | T/C |
| 9 | 31539325 | C/A |
| 9 | 31564038 | C/G |
| 9 | 31582524 | T/C |
| 9 | 31591248 | T/C |
| 9 | 31615845 | C/T |
| 9 | 31631247 | T/A |
| 9 | 31679166 | G/A |
| 9 | 31684849 | G/T |
| 9 | 31685931 | G/A |
| 9 | 31712684 | C/T |
| 9 | 31734082 | A/G |
| 9 | 31742393 | C/T |
| 9 | 31799681 | C/T |
| 9 | 31802610 | G/A |
| 9 | 31855295 | C/T |
| 9 | 31870217 | G/C |
| 9 | 31874745 | T/C |
| 9 | 31895010 | T/C |
| 9 | 31909920 | A/T |
| 9 | 31934018 | T/A |
| 9 | 31941133 | T/A |
| 9 | 31967474 | C/G |
| 9 | 31982881 | C/T |
| 9 | 31989760 | T/C |
| 9 | 32024797 | C/A |
| 9 | 32098646 | A/T |
| 9 | 32099910 | A/G |
| 9 | 32124558 | A/C |
| 9 | 32132901 | C/T |
| 9 | 32145423 | G/C |
| 9 | 32163087 | A/C |
| 9 | 32213778 | C/A |
| 9 | 32220013 | C/A |
| 9 | 32260586 | T/A |
| 9 | 32276852 | T/C |
| 9 | 32280991 | T/C |
| 9 | 32290185 | C/T |
| 9 | 32316382 | C/T |
| 9 | 32333645 | G/T |
| 9 | 32363666 | A/G |
| 9 | 32374273 | C/T |
| 9 | 32380705 | A/G |
| 9 | 32402049 | G/A |
| 9 | 32420602 | A/G |
| 9 | 32444974 | C/A |
| 9 | 32456695 | T/G |
| 9 | 32480819 | G/A |
| 9 | 32517123 | G/A |
| 9 | 32520065 | T/C |
| 9 | 32531772 | C/T |
| 9 | 32567257 | T/A |
| 9 | 32583928 | T/A |
| 9 | 32585149 | A/T |
| 9 | 32657213 | A/G |
| 9 | 32698751 | T/C |
| 9 | 32735362 | G/C |
| 9 | 32746794 | G/A |
| 9 | 32787936 | T/A |
| 9 | 32790745 | A/G |
| 9 | 32799384 | T/C |
| 9 | 32824180 | C/T |
| 9 | 32825768 | A/G |
| 9 | 32866173 | C/T |
| 9 | 32907209 | C/T |
| 9 | 32996013 | T/C |
| 9 | 33004611 | T/C |
| 9 | 33011083 | G/T |
| 9 | 33031506 | T/C |
| 9 | 33056981 | C/T |
| 9 | 33061096 | A/G |
| 9 | 33112820 | T/C |
| 9 | 33143275 | A/C |
| 9 | 33178607 | A/G |
| 9 | 33286890 | C/A |
| 9 | 33291046 | C/T |
| 9 | 33307130 | C/T |
| 9 | 33331909 | G/T |
| 9 | 33384600 | C/G |
| 9 | 33405910 | A/G |
| 9 | 33409869 | A/C |
| 9 | 33432735 | C/T |
| 9 | 33450126 | G/T |
| 9 | 33482035 | G/A |
| 9 | 33534679 | C/T |
| 9 | 33537458 | C/G |
| 9 | 33540380 | T/C |
| 9 | 33550781 | T/G |
| 9 | 33571366 | A/T |
| 9 | 33596222 | T/A |
| 9 | 33602533 | G/A |
| 9 | 33642815 | A/G |
| 9 | 33655603 | G/C |
| 9 | 33689521 | T/C |
| 9 | 33711524 | T/C |
| 9 | 33716587 | C/T |
| 9 | 33735130 | A/G |
| 9 | 33748483 | A/C |
| 9 | 33764582 | A/C |
| 9 | 33774526 | C/G |
| 9 | 33785864 | T/C |
| 9 | 33820555 | G/A |
| 9 | 33825622 | T/C |
| 9 | 33880484 | C/T |
| 9 | 33892403 | C/A |
| 9 | 33905501 | T/C |
| 9 | 33939402 | C/T |
| 9 | 33941034 | C/A |
| 9 | 33972928 | A/C |
| 9 | 33986626 | C/T |
| 9 | 33991620 | A/G |
| 9 | 34015356 | G/T |
| 9 | 34028600 | T/C |
| 9 | 34042283 | G/A |
| 9 | 34053490 | G/T |
| 9 | 34082669 | C/A |
| 9 | 34087101 | G/A |
| 9 | 34113068 | C/T |
| 9 | 34146634 | T/C |
| 9 | 34148264 | C/T |
| 9 | 34170038 | A/G |
| 9 | 34175383 | C/T |
| 9 | 34207641 | C/T |
| 9 | 34232613 | A/C |
| 9 | 34261225 | T/A |
| 9 | 34290759 | C/A |
| 9 | 34329815 | C/T |
| 9 | 34352667 | T/C |
| 9 | 34358175 | C/T |
| 9 | 34383718 | A/G |
| 9 | 34417148 | A/T |
| 9 | 34446023 | A/G |
| 9 | 34458742 | A/G |
| 9 | 34470306 | A/G |
| 10 | 56253 | G/A |
| 10 | 84260 | G/A |
| 10 | 103601 | A/G |
| 10 | 107924 | T/C |
| 10 | 142698 | T/A |
| 10 | 146205 | G/A |
| 10 | 151510 | G/A |
| 10 | 171015 | A/C |
| 10 | 182330 | C/T |
| 10 | 185087 | T/C |
| 10 | 212767 | G/T |
| 10 | 246564 | C/G |
| 10 | 273863 | G/A |
| 10 | 314364 | G/T |
| 10 | 321453 | A/G |
| 10 | 332725 | A/C |
| 10 | 362378 | A/T |
| 10 | 412809 | G/T |
| 10 | 442850 | C/A |
| 10 | 483371 | G/A |
| 10 | 519138 | G/A |
| 10 | 537424 | A/T |
| 10 | 540858 | G/T |
| 10 | 575753 | T/C |
| 10 | 593879 | A/G |
| 10 | 613746 | G/C |
| 10 | 623492 | T/C |
| 10 | 632908 | T/A |
| 10 | 667253 | G/A |
| 10 | 693900 | C/T |
| 10 | 721593 | C/T |
| 10 | 745479 | G/A |
| 10 | 752097 | A/T |
| 10 | 792065 | C/T |
| 10 | 808650 | G/T |
| 10 | 825206 | G/A |
| 10 | 831550 | C/G |
| 10 | 849880 | G/A |
| 10 | 865446 | C/T |
| 10 | 881151 | A/G |
| 10 | 887227 | T/C |
| 10 | 979426 | C/T |
| 10 | 999365 | A/C |
| 10 | 1025733 | A/C |
| 10 | 1069899 | C/T |
| 10 | 1081963 | C/T |
| 10 | 1150205 | C/T |
| 10 | 1186243 | G/A |
| 10 | 1210739 | G/C |
| 10 | 1225267 | C/T |
| 10 | 1233030 | G/A |
| 10 | 1238972 | G/A |
| 10 | 1265434 | C/G |
| 10 | 1280086 | G/C |
| 10 | 1357478 | T/C |
| 10 | 1360439 | A/G |
| 10 | 1385226 | T/C |
| 10 | 1415601 | A/T |
| 10 | 1466807 | T/G |
| 10 | 1478835 | A/G |
| 10 | 1501659 | A/G |
| 10 | 1544946 | C/A |
| 10 | 1585318 | G/A |
| 10 | 1589607 | T/C |
| 10 | 1600042 | G/A |
| 10 | 1617643 | G/T |
| 10 | 1622797 | T/C |
| 10 | 1648153 | A/G |
| 10 | 1663394 | C/G |
| 10 | 1708002 | T/C |
| 10 | 1736210 | T/G |
| 10 | 1754363 | C/T |
| 10 | 1798656 | T/C |
| 10 | 1839199 | G/A |
| 10 | 1882639 | C/T |
| 10 | 1886776 | A/T |
| 10 | 1900569 | A/C |
| 10 | 1920505 | T/G |
| 10 | 1923057 | C/T |
| 10 | 1967528 | A/C |
| 10 | 1975809 | C/T |
| 10 | 2001100 | G/A |
| 10 | 2039432 | A/T |
| 10 | 2069816 | C/G |
| 10 | 2099663 | C/T |
| 10 | 2107148 | T/C |
| 10 | 2140668 | C/T |
| 10 | 2178855 | C/T |
| 10 | 2201059 | G/A |
| 10 | 2209387 | G/A |
| 10 | 2221489 | C/T |
| 10 | 2237253 | G/T |
| 10 | 2253474 | C/T |
| 10 | 2274968 | A/C |
| 10 | 2303751 | T/A |
| 10 | 2318901 | C/T |
| 10 | 2354014 | G/A |
| 10 | 2360392 | A/G |
| 10 | 2391913 | A/T |
| 10 | 2404873 | T/C |
| 10 | 2422673 | T/C |
| 10 | 2457687 | G/T |
| 10 | 2506485 | G/A |
| 10 | 2534391 | G/T |
| 10 | 2565107 | G/A |
| 10 | 2602723 | T/A |
| 10 | 2614040 | A/C |
| 10 | 2641776 | T/C |
| 10 | 2643038 | T/C |
| 10 | 2676002 | G/C |
| 10 | 2690449 | C/T |
| 10 | 2717957 | C/T |
| 10 | 2726054 | C/A |
| 10 | 2740121 | A/C |
| 10 | 2747405 | C/T |
| 10 | 2777764 | G/T |
| 10 | 2812102 | T/G |
| 10 | 2820189 | C/T |
| 10 | 2831899 | C/T |
| 10 | 2861404 | A/G |
| 10 | 2907535 | G/A |
| 10 | 2913119 | G/T |
| 10 | 2950200 | G/T |
| 10 | 2987723 | A/T |
| 10 | 2997500 | G/T |
| 10 | 3000631 | A/C |
| 10 | 3034401 | G/A |
| 10 | 3060428 | T/A |
| 10 | 3064409 | T/C |
| 10 | 3103747 | C/T |
| 10 | 3128902 | C/A |
| 10 | 3152279 | G/T |
| 10 | 3156247 | T/C |
| 10 | 3189577 | T/C |
| 10 | 3218105 | T/C |
| 10 | 3234912 | G/A |
| 10 | 3240695 | C/G |
| 10 | 3274103 | A/G |
| 10 | 3278432 | T/G |
| 10 | 3317938 | G/T |
| 10 | 3386002 | T/C |
| 10 | 3390606 | A/G |
| 10 | 3404474 | T/G |
| 10 | 3423728 | T/C |
| 10 | 3471155 | G/A |
| 10 | 3481634 | C/T |
| 10 | 3503196 | G/A |
| 10 | 3514430 | G/C |
| 10 | 3517576 | A/C |
| 10 | 3540800 | T/C |
| 10 | 3559903 | A/G |
| 10 | 3584457 | C/G |
| 10 | 3605337 | G/A |
| 10 | 3623068 | T/C |
| 10 | 3630743 | T/A |
| 10 | 3634275 | C/G |
| 10 | 3662346 | G/A |
| 10 | 3724410 | T/G |
| 10 | 3728265 | T/G |
| 10 | 3750531 | G/C |
| 10 | 3762793 | C/T |
| 10 | 3809963 | A/C |
| 10 | 3836827 | A/T |
| 10 | 3844228 | T/G |
| 10 | 3872326 | T/C |
| 10 | 3915182 | T/A |
| 10 | 3943536 | C/T |
| 10 | 3958738 | C/G |
| 10 | 3966219 | C/T |
| 10 | 3987007 | G/A |
| 10 | 3991726 | G/A |
| 10 | 4034235 | C/T |
| 10 | 4067660 | C/T |
| 10 | 4088321 | G/T |
| 10 | 4119338 | G/A |
| 10 | 4123033 | T/A |
| 10 | 4212078 | G/A |
| 10 | 4228468 | A/G |
| 10 | 4251322 | A/C |
| 10 | 4267496 | G/A |
| 10 | 4327870 | C/A |
| 10 | 4419282 | T/C |
| 10 | 4461375 | A/C |
| 10 | 4469934 | C/T |
| 10 | 4477019 | T/C |
| 10 | 4505393 | G/T |
| 10 | 4527915 | T/C |
| 10 | 4550057 | G/T |
| 10 | 4577382 | G/T |
| 10 | 4577383 | T/C |
| 10 | 4577384 | T/C |
| 10 | 4577385 | T/C |
| 10 | 4634247 | C/T |
| 10 | 4669075 | C/T |
| 10 | 4686847 | C/T |
| 10 | 4702822 | A/G |
| 10 | 4702823 | G/A |
| 10 | 4702824 | G/A |
| 10 | 4702825 | G/A |
| 10 | 4766125 | G/T |
| 10 | 4782616 | A/T |
| 10 | 4857653 | G/T |
| 10 | 4898014 | G/C |
| 10 | 4926548 | G/T |
| 10 | 4961724 | C/T |
| 10 | 4963297 | T/G |
| 10 | 5003904 | C/T |
| 10 | 5063535 | C/G |
| 10 | 5068624 | A/C |
| 10 | 5072168 | C/T |
| 10 | 5085336 | T/C |
| 10 | 5103065 | C/G |
| 10 | 5108061 | T/G |
| 10 | 5130364 | A/T |
| 10 | 5166991 | G/A |
| 10 | 5210945 | G/C |
| 10 | 5242862 | C/T |
| 10 | 5248941 | C/T |
| 10 | 5255053 | G/A |
| 10 | 5310353 | C/T |
| 10 | 5329075 | T/C |
| 10 | 5346060 | C/T |
| 10 | 5359979 | T/C |
| 10 | 5426190 | C/T |
| 10 | 5436551 | G/A |
| 10 | 5438947 | T/C |
| 10 | 5509466 | T/A |
| 10 | 5606667 | T/C |
| 10 | 5612757 | T/C |
| 10 | 5620797 | A/T |
| 10 | 5654743 | T/C |
| 10 | 5671089 | T/C |
| 10 | 5703507 | T/C |
| 10 | 5740049 | A/G |
| 10 | 5760392 | G/A |
| 10 | 5784656 | G/A |
| 10 | 5793647 | G/A |
| 10 | 5802766 | C/T |
| 10 | 5826124 | T/C |
| 10 | 5851106 | T/C |
| 10 | 5875336 | T/A |
| 10 | 5898057 | T/A |
| 10 | 5906360 | G/C |
| 10 | 5926670 | C/A |
| 10 | 5952892 | T/A |
| 10 | 5966442 | C/A |
| 10 | 5970213 | T/C |
| 10 | 5974071 | G/A |
| 10 | 6000027 | A/G |
| 10 | 6016408 | T/A |
| 10 | 6038035 | G/C |
| 10 | 6052351 | A/C |
| 10 | 6061121 | C/A |
| 10 | 6069997 | C/T |
| 10 | 5109579 | C/A |
| 10 | 6127667 | T/A |
| 10 | 6171482 | T/C |
| 10 | 6186413 | G/C |
| 10 | 6200393 | C/T |
| 10 | 6234761 | A/G |
| 10 | 6264933 | C/T |
| 10 | 6284701 | G/A |
| 10 | 6302010 | C/G |
| 10 | 6308668 | C/G |
| 10 | 6336193 | C/T |
| 10 | 6388481 | T/G |
| 10 | 6389726 | G/A |
| 10 | 6390892 | C/T |
| 10 | 6396149 | C/T |
| 10 | 6422693 | G/C |
| 10 | 6460840 | G/T |
| 10 | 6469417 | T/C |
| 10 | 6493826 | A/T |
| 10 | 6519625 | T/C |
| 10 | 6535057 | C/T |
| 10 | 6540952 | T/C |
| 10 | 6542030 | C/T |
| 10 | 6577481 | T/C |
| 10 | 6586564 | A/G |
| 10 | 6610135 | C/T |
| 10 | 6651302 | C/A |
| 10 | 6668806 | G/A |
| 10 | 6681554 | T/C |
| 10 | 6719968 | G/A |
| 10 | 6733145 | C/A |
| 10 | 6736446 | T/C |
| 10 | 6775963 | T/G |
| 10 | 6780679 | A/G |
| 10 | 6797105 | C/T |
| 10 | 6829681 | A/G |
| 10 | 6831102 | T/G |
| 10 | 6847973 | G/T |
| 10 | 6860629 | G/A |
| 10 | 6924357 | G/A |
| 10 | 6942021 | A/G |
| 10 | 6999582 | A/T |
| 10 | 7021528 | C/T |
| 10 | 7077156 | T/C |
| 10 | 7093837 | A/C |
| 10 | 7103438 | T/A |
| 10 | 7126540 | A/G |
| 10 | 7134169 | A/G |
| 10 | 7150719 | T/A |
| 10 | 7170982 | G/T |
| 10 | 7216774 | G/A |
| 10 | 7251686 | T/A |
| 10 | 7255758 | C/T |
| 10 | 7267507 | C/T |
| 10 | 7269896 | G/C |
| 10 | 7299269 | G/T |
| 10 | 7311293 | A/G |
| 10 | 7340006 | C/T |
| 10 | 7346757 | A/G |
| 10 | 7361291 | C/T |
| 10 | 7406709 | T/A |
| 10 | 7415512 | C/T |
| 10 | 7475343 | C/T |
| 10 | 7502497 | T/C |
| 10 | 7525247 | T/C |
| 10 | 7535469 | C/T |
| 10 | 7538888 | C/G |
| 10 | 7577091 | T/G |
| 10 | 7588736 | T/C |
| 10 | 7600059 | G/A |
| 10 | 7605814 | G/A |
| 10 | 7649339 | T/C |
| 10 | 7650970 | G/C |
| 10 | 7709465 | T/C |
| 10 | 7731479 | T/A |
| 10 | 7778975 | A/C |
| 10 | 7825386 | A/G |
| 10 | 7859319 | C/T |
| 10 | 7875491 | G/A |
| 10 | 7905071 | G/A |
| 10 | 7906179 | C/A |
| 10 | 7965638 | C/T |
| 10 | 7984254 | G/A |
| 10 | 8006026 | G/A |
| 10 | 8028758 | G/A |
| 10 | 8039058 | G/C |
| 10 | 8069466 | C/A |
| 10 | 8076019 | G/A |
| 10 | 8098074 | T/C |
| 10 | 8100802 | A/G |
| 10 | 8114659 | G/A |
| 10 | 8140386 | A/T |
| 10 | 8166969 | C/G |
| 10 | 8199606 | C/T |
| 10 | 8214053 | C/G |
| 10 | 8230992 | G/A |
| 10 | 8250703 | G/A |
| 10 | 8253964 | A/C |
| 10 | 8291215 | A/T |
| 10 | 8293502 | C/A |
| 10 | 8314313 | C/T |
| 10 | 8334943 | T/A |
| 10 | 8340751 | G/A |
| 10 | 8370846 | T/C |
| 10 | 8372457 | A/G |
| 10 | 8403331 | C/T |
| 10 | 8432566 | G/A |
| 10 | 8447577 | T/G |
| 10 | 8483100 | G/A |
| 10 | 8491327 | C/T |
| 10 | 8521787 | A/G |
| 10 | 8565240 | T/C |
| 10 | 8598244 | G/T |
| 10 | 8611554 | C/T |
| 10 | 8616091 | T/C |
| 10 | 8641221 | T/C |
| 10 | 8678102 | T/A |
| 10 | 8695608 | C/G |
| 10 | 8701448 | T/C |
| 10 | 8710440 | G/C |
| 10 | 8757536 | G/A |
| 10 | 8779190 | C/T |
| 10 | 8786958 | G/A |
| 10 | 8790878 | A/C |
| 10 | 8814467 | T/C |
| 10 | 8828859 | T/G |
| 10 | 8850158 | A/T |
| 10 | 8877361 | C/T |
| 10 | 8885546 | C/T |
| 10 | 8911781 | G/A |
| 10 | 8931168 | A/G |
| 10 | 8945862 | G/A |
| 10 | 8966396 | C/T |
| 10 | 8988436 | C/T |
| 10 | 9019889 | C/T |
| 10 | 9023471 | A/C |
| 10 | 9032975 | C/G |
| 10 | 9039618 | C/A |
| 10 | 9073811 | A/C |
| 10 | 9087214 | G/A |
| 10 | 9161046 | C/A |
| 10 | 9195781 | T/C |
| 10 | 9217891 | T/A |
| 10 | 9263773 | C/T |
| 10 | 9341542 | C/T |
| 10 | 9343764 | T/A |
| 10 | 9376870 | A/G |
| 10 | 9398616 | T/C |
| 10 | 9401392 | A/G |
| 10 | 9435939 | A/T |
| 10 | 9460430 | A/G |
| 10 | 9463244 | C/T |
| 10 | 9482106 | C/G |
| 10 | 9514358 | G/A |
| 10 | 9536172 | T/C |
| 10 | 9574915 | C/T |
| 10 | 9670815 | G/C |
| 10 | 9689819 | T/C |
| 10 | 9693417 | C/G |
| 10 | 9718146 | G/A |
| 10 | 9726117 | C/T |
| 10 | 9727902 | A/G |
| 10 | 9754328 | A/G |
| 10 | 9763118 | A/G |
| 10 | 9780734 | G/C |
| 10 | 9782756 | G/A |
| 10 | 9814487 | T/A |
| 10 | 9826715 | C/T |
| 10 | 9858784 | C/T |
| 10 | 9868413 | C/T |
| 10 | 9872707 | A/G |
| 10 | 9876882 | G/A |
| 10 | 9915463 | T/C |
| 10 | 9947364 | C/T |
| 10 | 9974428 | A/T |
| 10 | 9977067 | A/G |
| 10 | 10014006 | C/T |
| 10 | 10028669 | A/G |
| 10 | 10030618 | C/T |
| 10 | 10077705 | C/A |
| 10 | 10087188 | A/G |
| 10 | 10090111 | A/G |
| 10 | 10136674 | T/C |
| 10 | 10143048 | T/G |
| 10 | 10131408 | G/A |
| 10 | 10175834 | G/A |
| 10 | 10186199 | T/C |
| 10 | 10203602 | G/A |
| 10 | 10214292 | A/T |
| 10 | 10238350 | T/A |
| 10 | 10255122 | A/G |
| 10 | 10261688 | C/T |
| 10 | 10280230 | T/C |
| 10 | 10298281 | A/C |
| 10 | 10301067 | C/A |
| 10 | 10347503 | T/G |
| 10 | 10354680 | T/A |
| 10 | 10385748 | T/G |
| 10 | 10422456 | T/C |
| 10 | 10428140 | A/G |
| 10 | 10443564 | G/A |
| 10 | 10449418 | A/C |
| 10 | 10480533 | A/G |
| 10 | 10495330 | A/G |
| 10 | 10506649 | C/A |
| 10 | 10508221 | T/G |
| 10 | 10531270 | A/G |
| 10 | 10577506 | A/C |
| 10 | 10604500 | C/T |
| 10 | 10620377 | T/A |
| 10 | 10621566 | A/G |
| 10 | 10666093 | G/A |
| 10 | 10702231 | C/T |
| 10 | 10716636 | G/A |
| 10 | 10747010 | G/T |
| 10 | 10770696 | A/G |
| 10 | 10806049 | G/A |
| 10 | 10807719 | A/G |
| 10 | 10859146 | G/A |
| 10 | 10873539 | A/G |
| 10 | 10878363 | G/T |
| 10 | 10905079 | C/G |
| 10 | 10914025 | G/A |
| 10 | 10934526 | A/G |
| 10 | 10951907 | T/C |
| 10 | 10988210 | G/A |
| 10 | 11019566 | A/C |
| 10 | 11021613 | A/T |
| 10 | 11047947 | T/G |
| 10 | 11063635 | T/C |
| 10 | 11093676 | A/G |
| 10 | 11106782 | A/G |
| 10 | 11134367 | C/T |
| 10 | 11178079 | G/A |
| 10 | 11208644 | A/G |
| 10 | 11215879 | A/G |
| 10 | 11229850 | T/C |
| 10 | 11236881 | C/A |
| 10 | 11259127 | A/C |
| 10 | 11280156 | T/C |
| 10 | 11296645 | A/G |
| 10 | 11332265 | A/G |
| 10 | 11338111 | C/T |
| 10 | 11340052 | G/C |
| 10 | 11346828 | T/C |
| 10 | 11394104 | T/G |
| 10 | 11406727 | G/C |
| 10 | 11430961 | G/A |
| 10 | 11439208 | C/A |
| 10 | 11486006 | G/T |
| 10 | 11487877 | A/G |
| 10 | 11506109 | A/G |
| 10 | 11520709 | T/C |
| 10 | 11523405 | C/T |
| 10 | 11562834 | T/C |
| 10 | 11578490 | A/G |
| 10 | 11594797 | C/G |
| 10 | 11603472 | T/C |
| 10 | 11610828 | T/C |
| 10 | 11623119 | A/G |
| 10 | 11653247 | G/T |
| 10 | 11673684 | C/T |
| 10 | 11679045 | G/T |
| 10 | 11725465 | A/G |
| 10 | 11728839 | T/C |
| 10 | 11743616 | C/T |
| 10 | 11747943 | A/T |
| 10 | 11781356 | C/T |
| 10 | 11797500 | C/T |
| 10 | 11840926 | A/G |
| 10 | 11843034 | C/T |
| 10 | 11850980 | A/T |
| 10 | 11855519 | C/G |
| 10 | 11881248 | T/C |
| 10 | 11884103 | G/A |
| 10 | 11936286 | A/G |
| 10 | 11957206 | A/C |
| 10 | 11988868 | G/A |
| 10 | 11995468 | G/A |
| 10 | 12020078 | C/T |
| 10 | 12030383 | G/C |
| 10 | 12057305 | A/G |
| 10 | 12064300 | G/A |
| 10 | 12074260 | T/C |
| 10 | 12092352 | G/C |
| 10 | 12116042 | G/A |
| 10 | 12123730 | A/T |
| 10 | 12155491 | C/T |
| 10 | 12178769 | C/G |
| 10 | 12196535 | C/G |
| 10 | 12210086 | A/T |
| 10 | 12227100 | C/A |
| 10 | 12258046 | C/T |
| 10 | 12291676 | G/A |
| 10 | 12301258 | C/A |
| 10 | 12308731 | A/T |
| 10 | 12335860 | A/C |
| 10 | 12367845 | C/G |
| 10 | 12375729 | C/G |
| 10 | 12412663 | G/A |
| 10 | 12420164 | C/T |
| 10 | 12432922 | T/C |
| 10 | 12457381 | A/T |
| 10 | 12497771 | G/A |
| 10 | 12504091 | A/G |
| 10 | 12513059 | C/T |
| 10 | 12514230 | A/T |
| 10 | 12540591 | C/T |
| 10 | 12571695 | C/A |
| 10 | 12600057 | T/G |
| 10 | 12624504 | T/A |
| 10 | 12636122 | C/T |
| 10 | 12657227 | A/T |
| 10 | 12666375 | G/A |
| 10 | 12686368 | C/T |
| 10 | 12697855 | C/A |
| 10 | 12728177 | T/C |
| 10 | 12731558 | G/A |
| 10 | 12761711 | G/A |
| 10 | 12793039 | T/C |
| 10 | 12805154 | C/T |
| 10 | 12820552 | A/T |
| 10 | 12832883 | G/A |
| 10 | 12843015 | G/A |
| 10 | 12849158 | C/T |
| 10 | 12877098 | A/G |
| 10 | 12901609 | G/C |
| 10 | 12953461 | G/A |
| 10 | 12979717 | G/T |
| 10 | 12981794 | G/C |
| 10 | 12994438 | G/A |
| 10 | 13000794 | G/C |
| 10 | 13026341 | A/C |
| 10 | 13124997 | A/G |
| 10 | 13154562 | T/G |
| 10 | 13170437 | C/T |
| 10 | 13204228 | G/C |
| 10 | 13232513 | A/C |
| 10 | 13233692 | T/C |
| 10 | 13289537 | A/T |
| 10 | 13295653 | G/A |
| 10 | 13306784 | T/C |
| 10 | 13332823 | A/G |
| 10 | 13350047 | G/A |
| 10 | 13362659 | A/G |
| 10 | 13380847 | A/C |
| 10 | 13410864 | T/C |
| 10 | 13411894 | T/C |
| 10 | 13455275 | G/A |
| 10 | 13473681 | T/C |
| 10 | 13519082 | C/T |
| 10 | 13537547 | C/A |
| 10 | 13540495 | C/T |
| 10 | 13564284 | C/T |
| 10 | 13577145 | C/T |
| 10 | 13593920 | G/C |
| 10 | 13635293 | C/T |
| 10 | 13661777 | G/A |
| 10 | 13674382 | T/A |
| 10 | 13690643 | T/C |
| 10 | 13725891 | T/A |
| 10 | 13736982 | C/A |
| 10 | 13769137 | C/T |
| 10 | 13771350 | A/C |
| 10 | 13795058 | C/G |
| 10 | 13814355 | A/G |
| 10 | 13814356 | C/T |
| 10 | 13814357 | C/T |
| 10 | 13814358 | C/T |
| 10 | 13887077 | T/A |
| 10 | 13911271 | C/G |
| 10 | 13919619 | C/G |
| 10 | 13928342 | A/C |
| 10 | 13948447 | G/A |
| 10 | 13979389 | A/C |
| 10 | 14040460 | C/T |
| 10 | 14083210 | G/A |
| 10 | 14101982 | T/G |
| 10 | 14113443 | A/G |
| 10 | 14189025 | G/A |
| 10 | 14190250 | T/C |
| 10 | 14210790 | G/A |
| 10 | 14246346 | C/T |
| 10 | 14287715 | A/G |
| 10 | 14311362 | C/T |
| 10 | 14318959 | T/C |
| 10 | 14399347 | T/A |
| 10 | 14440924 | C/T |
| 10 | 14478759 | T/G |
| 10 | 14489655 | C/T |
| 10 | 14509622 | G/A |
| 10 | 14555625 | C/A |
| 10 | 14578456 | A/G |
| 10 | 14597751 | G/A |
| 10 | 14620199 | G/A |
| 10 | 14646254 | T/C |
| 10 | 14666252 | T/A |
| 10 | 14671771 | C/A |
| 10 | 14749359 | T/C |
| 10 | 14758136 | C/T |
| 10 | 14764815 | C/T |
| 10 | 14811998 | G/A |
| 10 | 14886082 | G/A |
| 10 | 14887928 | T/C |
| 10 | 14927487 | C/T |
| 10 | 14946911 | T/C |
| 10 | 14988346 | A/G |
| 10 | 15036940 | T/A |
| 10 | 15062176 | T/C |
| 10 | 15092505 | C/T |
| 10 | 15105760 | T/C |
| 10 | 15132547 | G/A |
| 10 | 15147688 | T/C |
| 10 | 15162002 | G/A |
| 10 | 15170649 | C/A |
| 10 | 15185082 | G/A |
| 10 | 15227155 | A/G |
| 10 | 15277853 | T/C |
| 10 | 15342692 | A/T |
| 10 | 15395376 | C/T |
| 10 | 15404486 | C/T |
| 10 | 15432603 | C/T |
| 10 | 15474444 | T/G |
| 10 | 15492951 | G/A |
| 10 | 15501444 | T/C |
| 10 | 15521192 | T/C |
| 10 | 15569158 | C/T |
| 10 | 15596265 | C/A |
| 10 | 15626956 | T/A |
| 10 | 15646599 | A/G |
| 10 | 15685226 | C/T |
| 10 | 15688136 | T/C |
| 10 | 15708146 | A/T |
| 10 | 15721538 | T/G |
| 10 | 15755931 | G/A |
| 10 | 15760142 | C/T |
| 10 | 15795161 | A/G |
| 10 | 15856117 | G/A |
| 10 | 15903000 | C/T |
| 10 | 15940622 | A/G |
| 10 | 15986963 | C/T |
| 10 | 15994283 | G/A |
| 10 | 16042974 | C/G |
| 10 | 16064874 | T/A |
| 10 | 16097195 | A/G |
| 10 | 16129230 | G/C |
| 10 | 16136596 | T/C |
| 10 | 16156145 | T/G |
| 10 | 16161497 | A/G |
| 10 | 16204633 | T/G |
| 10 | 16397452 | A/G |
| 10 | 16404272 | G/A |
| 10 | 16410021 | C/A |
| 10 | 16419205 | T/C |
| 10 | 16443281 | T/C |
| 10 | 16492581 | G/A |
| 10 | 16535628 | G/A |
| 10 | 16552055 | G/A |
| 10 | 16572737 | A/G |
| 10 | 16587168 | A/C |
| 10 | 16601611 | T/C |
| 10 | 16607557 | G/C |
| 10 | 16657441 | G/A |
| 10 | 16686926 | A/G |
| 10 | 16723125 | T/A |
| 10 | 16753985 | A/T |
| 10 | 16862602 | C/A |
| 10 | 16875044 | G/A |
| 10 | 16894780 | A/G |
| 10 | 16928421 | G/C |
| 10 | 16934217 | A/G |
| 10 | 16970430 | G/C |
| 10 | 16980855 | G/A |
| 10 | 17020466 | T/C |
| 10 | 17039901 | C/T |
| 10 | 17047673 | G/A |
| 10 | 17103843 | C/T |
| 10 | 17127270 | T/C |
| 10 | 17130152 | C/T |
| 10 | 17138890 | C/T |
| 10 | 17188429 | A/C |
| 10 | 17198825 | C/T |
| 10 | 17201026 | T/G |
| 10 | 17227178 | A/T |
| 10 | 17247819 | C/T |
| 10 | 17274169 | C/A |
| 10 | 17336382 | A/G |
| 10 | 17343065 | T/C |
| 10 | 17392680 | C/T |
| 10 | 17405537 | A/G |
| 10 | 17468624 | G/A |
| 10 | 17480536 | G/A |
| 10 | 17491790 | G/A |
| 10 | 17558089 | G/T |
| 10 | 17580168 | C/T |
| 10 | 17599877 | C/T |
| 10 | 17610128 | G/A |
| 10 | 17637882 | C/T |
| 10 | 17647218 | T/G |
| 10 | 17667114 | C/T |
| 10 | 17718296 | T/C |
| 10 | 17817422 | T/C |
| 10 | 17830916 | C/T |
| 10 | 17846568 | T/A |
| 10 | 17895712 | T/A |
| 10 | 17898954 | G/A |
| 10 | 17920573 | G/A |
| 10 | 17933819 | G/A |
| 10 | 17970938 | G/A |
| 10 | 18025346 | C/T |
| 10 | 18044017 | G/A |
| 10 | 18071501 | A/G |
| 10 | 18102730 | T/A |
| 10 | 18104090 | C/T |
| 10 | 18127565 | A/G |
| 10 | 18171604 | G/A |
| 10 | 18177203 | C/T |
| 10 | 18184813 | T/C |
| 10 | 18199468 | G/A |
| 10 | 18265516 | C/A |
| 10 | 18295226 | T/C |
| 10 | 18317338 | G/T |
| 10 | 18354348 | A/G |
| 10 | 18380471 | C/T |
| 10 | 18417769 | G/A |
| 10 | 18421269 | G/A |
| 10 | 18439329 | T/A |
| 10 | 18454011 | G/A |
| 10 | 18457721 | G/A |
| 10 | 18507562 | G/A |
| 10 | 18533339 | A/G |
| 10 | 18558645 | G/A |
| 10 | 18600293 | A/C |
| 10 | 18611477 | A/G |
| 10 | 18637801 | C/T |
| 10 | 18642550 | G/A |
| 10 | 18662240 | A/T |
| 10 | 18738270 | G/A |
| 10 | 18779645 | C/A |
| 10 | 18812328 | C/T |
| 10 | 18830673 | G/A |
| 10 | 18880153 | G/T |
| 10 | 18881944 | G/A |
| 10 | 18926676 | C/G |
| 10 | 18956401 | G/A |
| 10 | 18967800 | G/A |
| 10 | 19001294 | C/T |
| 10 | 19013357 | G/A |
| 10 | 19020058 | C/T |
| 10 | 19047886 | A/G |
| 10 | 19143248 | A/C |
| 10 | 19156898 | C/T |
| 10 | 19172391 | T/C |
| 10 | 19199596 | G/A |
| 10 | 19206849 | G/A |
| 10 | 19261483 | C/T |
| 10 | 19492589 | C/T |
| 10 | 19506721 | G/A |
| 10 | 19528349 | G/T |
| 10 | 19545024 | G/A |
| 10 | 19547645 | G/A |
| 10 | 19579349 | A/C |
| 10 | 19616008 | G/A |
| 10 | 19629177 | A/G |
| 10 | 19688222 | T/C |
| 10 | 19719134 | G/A |
| 10 | 19728872 | A/T |
| 10 | 19810859 | G/C |
| 10 | 19831617 | G/A |
| 10 | 19846627 | G/A |
| 10 | 19864986 | T/G |
| 10 | 19897642 | T/A |
| 10 | 19909327 | C/T |
| 10 | 19932435 | C/T |
| 10 | 19944046 | G/A |
| 10 | 19976277 | G/A |
| 10 | 19987595 | T/C |
| 10 | 20005574 | C/T |
| 10 | 20011126 | C/T |
| 10 | 20041715 | C/T |
| 10 | 20073355 | T/C |
| 10 | 20122353 | T/G |
| 10 | 20138670 | C/T |
| 10 | 20140770 | C/T |
| 10 | 20160070 | T/C |
| 10 | 20164365 | G/A |
| 10 | 20197195 | G/A |
| 10 | 20224278 | G/A |
| 10 | 20271284 | A/T |
| 10 | 20290318 | T/C |
| 10 | 20321769 | G/T |
| 10 | 20325905 | T/C |
| 10 | 20348098 | T/C |
| 10 | 20356307 | G/A |
| 10 | 20370433 | A/T |
| 10 | 20404717 | C/T |
| 10 | 20455393 | G/A |
| 10 | 20479402 | G/A |
| 10 | 20497152 | C/T |
| 10 | 20577378 | C/T |
| 10 | 20603425 | C/G |
| 10 | 20648967 | C/G |
| 10 | 20662680 | G/A |
| 10 | 20686987 | T/A |
| 10 | 20756694 | G/A |
| 10 | 20773528 | G/A |
| 10 | 20775832 | G/A |
| 10 | 20815817 | G/A |
| 10 | 20842773 | A/C |
| 10 | 20843905 | G/A |
| 10 | 20909254 | C/T |
| 10 | 20915620 | G/A |
| 10 | 20919833 | C/T |
| 10 | 20955735 | C/T |
| 10 | 20966830 | T/C |
| 10 | 20979826 | A/G |
| 10 | 21043545 | T/C |
| 10 | 21076805 | G/A |
| 10 | 21086263 | G/A |
| 10 | 21116384 | C/T |
| 10 | 21123156 | G/A |
| 10 | 21129738 | C/T |
| 10 | 21162630 | C/T |
| 10 | 21167268 | C/A |
| 10 | 21194828 | G/A |
| 10 | 21204970 | C/T |
| 10 | 21218928 | G/A |
| 10 | 21266630 | G/A |
| 10 | 21324252 | G/A |
| 10 | 21417286 | T/C |
| 10 | 21468924 | T/A |
| 10 | 21485870 | T/C |
| 10 | 21514228 | G/A |
| 10 | 21530192 | C/G |
| 10 | 21543969 | G/A |
| 10 | 21576766 | G/A |
| 10 | 21588627 | G/A |
| 10 | 21604916 | G/A |
| 10 | 21640629 | G/A |
| 10 | 21669637 | C/T |
| 10 | 21680265 | T/A |
| 10 | 21700990 | T/C |
| 10 | 21774472 | T/G |
| 10 | 21787932 | C/T |
| 10 | 21846923 | A/G |
| 10 | 21874239 | T/C |
| 10 | 21905144 | C/G |
| 10 | 21920353 | T/C |
| 10 | 21931173 | G/T |
| 10 | 21935582 | C/T |
| 10 | 21969257 | C/T |
| 10 | 21997365 | C/G |
| 10 | 22042325 | T/A |
| 10 | 22050787 | T/A |
| 10 | 22064212 | A/G |
| 10 | 22102418 | A/C |
| 10 | 22106752 | C/T |
| 10 | 22128879 | T/C |
| 10 | 22152960 | A/G |
| 10 | 22156771 | C/A |
| 10 | 22184732 | C/T |
| 10 | 22186125 | G/A |
| 10 | 22256036 | A/G |
| 10 | 22276051 | C/G |
| 10 | 22307959 | G/T |
| 10 | 22335968 | G/C |
| 10 | 22355710 | C/T |
| 10 | 22386871 | G/A |
| 10 | 22425460 | T/C |
| 10 | 22427145 | T/G |
| 10 | 22445222 | T/C |
| 10 | 22500771 | C/T |
| 10 | 22517425 | G/A |
| 10 | 22550979 | T/C |
| 10 | 22568623 | G/A |
| 10 | 22605639 | A/G |
| 10 | 22627353 | T/C |
| 10 | 22665954 | G/C |
| 10 | 22686170 | A/G |
| 10 | 22733574 | A/G |
| 10 | 22772222 | T/C |
| 10 | 22776883 | A/G |
| 10 | 22835198 | G/C |
| 10 | 22837287 | A/G |
| 10 | 22892787 | C/T |
| 10 | 22917085 | C/T |
| 10 | 22927481 | T/C |
| 10 | 22951088 | A/G |
| 10 | 22962725 | C/T |
| 10 | 22997675 | T/C |
| 10 | 23024448 | C/T |
| 10 | 23043523 | G/A |
| 10 | 23055222 | T/C |
| 10 | 23130899 | T/C |
| 10 | 23179477 | T/C |
| 10 | 23183260 | C/T |
| 10 | 23195140 | G/A |
| 10 | 23222478 | C/T |
| 10 | 23225512 | A/T |
| 10 | 23250173 | C/T |
| 10 | 23272966 | G/A |
| 10 | 23283326 | C/A |
| 10 | 23284571 | T/A |
| 10 | 23321734 | A/G |
| 10 | 23334161 | T/C |
| 10 | 23368969 | A/T |
| 10 | 23372255 | G/C |
| 10 | 23375632 | G/T |
| 10 | 23425858 | A/T |
| 10 | 23441376 | A/T |
| 10 | 23471170 | G/T |
| 10 | 23489388 | T/C |
| 10 | 23498303 | T/C |
| 10 | 23504681 | C/G |
| 10 | 23523353 | A/G |
| 10 | 23539605 | G/A |
| 10 | 23569640 | G/A |
| 10 | 23597429 | G/T |
| 10 | 23604117 | G/T |
| 10 | 23639746 | T/C |
| 10 | 23658056 | G/A |
| 10 | 23664265 | G/A |
| 10 | 23677742 | T/A |
| 10 | 23746883 | G/A |
| 10 | 23759665 | A/C |
| 10 | 23760803 | G/A |
| 10 | 23765742 | A/G |
| 10 | 23794665 | A/G |
| 10 | 23826428 | T/A |
| 10 | 23837003 | G/A |
| 10 | 23859321 | C/G |
| 10 | 23894363 | G/A |
| 10 | 23910003 | T/C |
| 10 | 23931486 | A/G |
| 10 | 23940071 | A/T |
| 10 | 23963066 | T/G |
| 10 | 23977090 | A/G |
| 10 | 23990957 | A/G |
| 10 | 24008451 | T/C |
| 10 | 24035786 | A/G |
| 10 | 24051611 | T/C |
| 10 | 24063774 | A/C |
| 10 | 24070991 | T/C |
| 10 | 24110060 | C/G |
| 10 | 24111094 | C/G |
| 10 | 24140688 | A/G |
| 10 | 24143425 | C/T |
| 10 | 24158487 | T/C |
| 10 | 24188709 | C/T |
| 10 | 24211290 | T/G |
| 10 | 24234344 | A/T |
| 10 | 24252912 | G/C |
| 10 | 24268804 | A/G |
| 10 | 24281005 | A/G |
| 10 | 24298606 | C/T |
| 10 | 24324635 | C/T |
| 10 | 24346791 | T/C |
| 10 | 24378241 | T/C |
| 10 | 24416838 | T/C |
| 10 | 24449707 | A/G |
| 10 | 24464373 | T/C |
| 10 | 24484180 | G/C |
| 10 | 24506823 | G/A |
| 10 | 24519120 | A/G |
| 10 | 24568640 | T/C |
| 10 | 24653142 | T/C |
| 10 | 24681384 | A/G |
| 10 | 14693239 | C/T |
| 10 | 24724888 | G/A |
| 10 | 24748842 | A/G |
| 10 | 24754657 | C/T |
| 10 | 24781224 | C/T |
| 10 | 24821177 | C/T |
| 10 | 24827703 | G/T |
| 10 | 24853233 | G/A |
| 10 | 24899571 | C/T |
| 10 | 24917353 | G/A |
| 10 | 24932606 | G/A |
| 10 | 24951484 | G/A |
| 10 | 24979364 | A/G |
| 10 | 25001419 | A/G |
| 10 | 25021914 | C/T |
| 10 | 25044268 | T/G |
| 10 | 25060310 | T/C |
| 10 | 25077165 | C/T |
| 10 | 25084520 | T/A |
| 10 | 25095216 | C/G |
| 10 | 25124759 | A/C |
| 10 | 25146495 | A/C |
| 10 | 25176206 | C/T |
| 10 | 25237885 | G/A |
| 10 | 25296533 | A/G |
| 10 | 25311658 | A/G |
| 10 | 25369012 | G/A |
| 10 | 25389543 | A/G |
| 10 | 25412705 | C/T |
| 10 | 25433272 | A/G |
| 10 | 25458621 | C/T |
| 10 | 25490666 | G/A |
| 10 | 25508091 | T/C |
| 10 | 25513617 | C/T |
| 10 | 25594903 | G/A |
| 10 | 25635284 | C/T |
| 10 | 25693349 | G/A |
| 10 | 25721755 | C/T |
| 10 | 25743075 | G/A |
| 10 | 25842070 | A/G |
| 10 | 25889178 | C/T |
| 10 | 25892884 | G/A |
| 10 | 25934575 | T/C |
| 10 | 25974097 | C/T |
| 10 | 26005245 | G/T |
| 10 | 26007438 | T/C |
| 10 | 26016125 | C/T |
| 10 | 26052780 | T/C |
| 10 | 26059010 | T/G |
| 10 | 26080167 | C/T |
| 10 | 26086729 | A/G |
| 10 | 26124965 | T/C |
| 10 | 26141863 | T/A |
| 10 | 26149725 | T/G |
| 10 | 26164999 | T/A |
| 10 | 26171389 | A/C |
| 10 | 26193255 | T/G |
| 10 | 26207869 | G/A |
| 10 | 26238250 | A/G |
| 10 | 26309051 | C/T |
| 10 | 26310607 | G/C |
| 10 | 26320492 | T/C |
| 10 | 26341945 | G/A |
| 10 | 26369492 | G/A |
| 10 | 26418276 | C/A |
| 10 | 26454364 | G/A |
| 10 | 26455787 | T/C |
| 10 | 26503367 | G/C |
| 10 | 26533871 | C/T |
| 10 | 26581033 | C/T |
| 10 | 26601302 | T/G |
| 10 | 26624280 | C/T |
| 10 | 26635311 | C/T |
| 10 | 26643648 | C/T |
| 10 | 26694077 | C/T |
| 10 | 26699273 | G/A |
| 10 | 26726199 | G/A |
| 10 | 26744648 | G/A |
| 10 | 26759020 | C/G |
| 10 | 26767390 | A/C |
| 10 | 26786970 | G/A |
| 10 | 26821555 | A/C |
| 10 | 26854870 | G/T |
| 10 | 26864767 | G/A |
| 10 | 26883645 | T/C |
| 10 | 26912096 | G/T |
| 10 | 26933268 | A/C |
| 10 | 26961455 | C/G |
| 10 | 26972987 | T/C |
| 10 | 27021746 | G/C |
| 10 | 27089334 | A/G |
| 10 | 27117988 | A/G |
| 10 | 27127812 | C/T |
| 10 | 27146323 | G/A |
| 10 | 27171560 | T/G |
| 10 | 27176729 | A/C |
| 10 | 27186129 | A/G |
| 10 | 27228662 | C/A |
| 10 | 27236031 | A/T |
| 10 | 27241257 | T/C |
| 10 | 27266668 | T/C |
| 10 | 27270636 | T/C |
| 10 | 27296498 | A/G |
| 10 | 27335427 | A/C |
| 10 | 27353074 | G/C |
| 10 | 27413583 | C/T |
| 10 | 27419502 | G/A |
| 10 | 27422294 | G/A |
| 10 | 27453066 | T/G |
| 10 | 27464437 | T/C |
| 10 | 27490076 | G/C |
| 10 | 27495227 | G/T |
| 10 | 27519361 | C/G |
| 10 | 27522682 | G/A |
| 10 | 27554088 | G/C |
| 10 | 27565395 | A/G |
| 10 | 27574157 | T/C |
| 10 | 27597884 | G/A |
| 10 | 27604872 | C/T |
| 10 | 27634262 | G/C |
| 10 | 27668676 | C/T |
| 10 | 27707558 | A/G |
| 10 | 27731542 | C/G |
| 10 | 27784166 | T/C |
| 10 | 27793060 | A/G |
| 10 | 27844609 | A/G |
| 10 | 27882692 | G/A |
| 10 | 27920971 | A/G |
| 10 | 27935598 | C/T |
| 10 | 27959995 | G/A |
| 10 | 27961028 | G/A |
| 10 | 27979533 | G/A |
| 10 | 27993054 | G/A |
| 10 | 28016786 | C/A |
| 10 | 28046269 | A/G |
| 10 | 28070093 | C/T |
| 10 | 28105476 | G/T |
| 10 | 28106515 | C/A |
| 10 | 28156969 | G/A |
| 10 | 28167847 | C/T |
| 10 | 28193068 | T/C |
| 10 | 28278909 | G/A |
| 10 | 28368801 | C/T |
| 10 | 28427000 | A/G |
| 10 | 28452875 | G/T |
| 10 | 28484193 | G/A |
| 10 | 28504010 | C/T |
| 10 | 28528101 | C/T |
| 10 | 28530888 | G/C |
| 10 | 28563331 | C/T |
| 10 | 28564972 | A/G |
| 10 | 28659251 | G/A |
| 10 | 28683612 | G/A |
| 10 | 28713669 | G/A |
| 10 | 28731502 | A/T |
| 10 | 28769444 | C/A |
| 10 | 28791244 | A/G |
| 10 | 28829837 | C/T |
| 10 | 28837359 | A/T |
| 10 | 28860766 | G/A |
| 10 | 28881355 | G/A |
| 10 | 28894355 | G/A |
| 10 | 28918916 | G/A |
| 10 | 28932009 | C/T |
| 10 | 28950138 | A/G |
| 10 | 28990280 | T/C |
| 10 | 29002783 | G/A |
| 10 | 29016445 | G/A |
| 10 | 29033353 | T/C |
| 10 | 29043523 | T/C |
| 10 | 29066167 | T/C |
| 10 | 29081204 | T/G |
| 10 | 29104929 | T/C |
| 10 | 29121369 | G/A |
| 10 | 29134189 | A/G |
| 10 | 29140178 | G/A |
| 10 | 29164867 | T/C |
| 10 | 29238106 | G/A |
| 10 | 29281784 | G/A |
| 10 | 29308290 | A/G |
| 10 | 29356681 | T/A |
| 10 | 29358984 | C/T |
| 10 | 29455961 | G/A |
| 10 | 29472074 | T/C |
| 10 | 29501962 | G/A |
| 10 | 29508663 | C/T |
| 10 | 29545947 | A/T |
| 10 | 29570196 | C/A |
| 10 | 29648092 | G/A |
| 10 | 29670754 | C/G |
| 10 | 29707387 | C/G |
| 10 | 29718079 | C/T |
| 10 | 29736042 | T/G |
| 10 | 29774993 | A/G |
| 10 | 29784659 | T/A |
| 10 | 29815529 | C/T |
| 10 | 29835853 | A/T |
| 10 | 29907143 | C/T |
| 10 | 29916076 | G/A |
| 10 | 29997585 | C/T |
| 10 | 30018135 | C/A |
| 10 | 30036581 | T/C |
| 10 | 30044614 | G/A |
| 10 | 30087513 | G/A |
| 11 | 39154 | C/T |
| 11 | 58866 | T/A |
| 11 | 88343 | C/T |
| 11 | 115740 | G/T |
| 11 | 122504 | C/T |
| 11 | 130547 | C/T |
| 11 | 150280 | G/A |
| 11 | 203036 | C/T |
| 11 | 210977 | G/A |
| 11 | 243681 | G/A |
| 11 | 311184 | C/T |
| 11 | 322169 | A/G |
| 11 | 355651 | T/C |
| 11 | 366299 | A/G |
| 11 | 396361 | G/A |
| 11 | 420069 | A/T |
| 11 | 450085 | G/A |
| 11 | 484995 | C/T |
| 11 | 490119 | C/T |
| 11 | 511222 | C/T |
| 11 | 538115 | T/C |
| 11 | 550267 | G/A |
| 11 | 587324 | T/C |
| 11 | 618813 | G/A |
| 11 | 633371 | G/A |
| 11 | 663291 | T/C |
| 11 | 711162 | A/G |
| 11 | 720156 | C/T |
| 11 | 760396 | C/G |
| 11 | 769772 | T/C |
| 11 | 794307 | C/G |
| 11 | 821394 | G/A |
| 11 | 827229 | C/G |
| 11 | 845748 | T/C |
| 11 | 864832 | T/A |
| 11 | 870612 | C/T |
| 11 | 877850 | T/A |
| 11 | 901875 | A/G |
| 11 | 903296 | T/C |
| 11 | 931875 | A/T |
| 11 | 946565 | A/G |
| 11 | 988258 | A/G |
| 11 | 999584 | T/G |
| 11 | 1031958 | G/T |
| 11 | 1042857 | C/G |
| 11 | 1050558 | T/G |
| 11 | 1059205 | A/G |
| 11 | 1080608 | T/C |
| 11 | 1100578 | C/T |
| 11 | 1114125 | A/C |
| 11 | 1121175 | T/C |
| 11 | 1140264 | C/T |
| 11 | 1181869 | C/T |
| 11 | 1185383 | C/A |
| 11 | 1203501 | C/A |
| 11 | 1224990 | C/T |
| 11 | 1231006 | A/C |
| 11 | 1234211 | G/A |
| 11 | 1276111 | G/A |
| 11 | 1312981 | A/T |
| 11 | 1335832 | G/A |
| 11 | 1368338 | T/C |
| 11 | 1376334 | C/G |
| 11 | 1386412 | C/A |
| 11 | 1414507 | C/A |
| 11 | 1457234 | A/G |
| 11 | 1494093 | C/T |
| 11 | 1498816 | A/T |
| 11 | 1500057 | C/G |
| 11 | 1518581 | A/C |
| 11 | 1531034 | C/T |
| 11 | 1552943 | A/G |
| 11 | 1563581 | T/C |
| 11 | 1591494 | G/T |
| 11 | 1634648 | G/A |
| 11 | 1637361 | A/G |
| 11 | 1672292 | C/T |
| 11 | 1703613 | G/T |
| 11 | 1706905 | C/T |
| 11 | 1713357 | A/C |
| 11 | 1732777 | C/A |
| 11 | 1752896 | G/A |
| 11 | 1767259 | T/A |
| 11 | 1770579 | C/A |
| 11 | 1772563 | A/C |
| 11 | 1813226 | T/C |
| 11 | 1836226 | C/G |
| 11 | 1845550 | G/A |
| 11 | 1886172 | A/C |
| 11 | 1916317 | G/A |
| 11 | 1920223 | C/T |
| 11 | 1951673 | A/G |
| 11 | 1958270 | A/T |
| 11 | 1984059 | C/T |
| 11 | 2006546 | C/T |
| 11 | 2019757 | G/A |
| 11 | 2052711 | A/G |
| 11 | 2062013 | C/T |
| 11 | 2078268 | A/G |
| 11 | 2094016 | G/A |
| 11 | 2124832 | T/C |
| 11 | 2127083 | G/A |
| 11 | 2131056 | A/T |
| 11 | 2133859 | A/G |
| 11 | 2161560 | G/A |
| 11 | 2163923 | A/C |
| 11 | 2219655 | T/A |
| 11 | 2224678 | A/G |
| 11 | 2236627 | C/T |
| 11 | 2254593 | A/T |
| 11 | 2280141 | A/G |
| 11 | 2294378 | G/A |
| 11 | 2310624 | T/A |
| 11 | 2370692 | A/C |
| 11 | 2394446 | C/G |
| 11 | 2410613 | A/G |
| 11 | 2415799 | T/G |
| 11 | 2431012 | C/T |
| 11 | 2486890 | A/T |
| 11 | 2490231 | T/A |
| 11 | 2514416 | G/A |
| 11 | 2540606 | C/G |
| 11 | 2543965 | G/A |
| 11 | 2572673 | C/G |
| 11 | 2580917 | A/C |
| 11 | 2607580 | T/A |
| 11 | 2632187 | T/C |
| 11 | 2639748 | G/A |
| 11 | 2651076 | A/T |
| 11 | 2656728 | G/A |
| 11 | 2670199 | A/G |
| 11 | 2706510 | C/A |
| 11 | 2709694 | T/C |
| 11 | 2743960 | T/C |
| 11 | 2760020 | T/G |
| 11 | 2780895 | A/T |
| 11 | 2798904 | G/A |
| 11 | 2827215 | C/T |
| 11 | 2832172 | T/C |
| 11 | 2861774 | G/A |
| 11 | 2880950 | T/G |
| 11 | 2888202 | C/T |
| 11 | 2927873 | G/A |
| 11 | 2941548 | A/G |
| 11 | 2950807 | G/C |
| 11 | 2982709 | A/G |
| 11 | 3029932 | A/C |
| 11 | 3035541 | A/G |
| 11 | 3068482 | G/A |
| 11 | 3079605 | A/G |
| 11 | 3111076 | C/T |
| 11 | 3134390 | T/C |
| 11 | 3173269 | C/T |
| 11 | 3181898 | G/T |
| 11 | 3190959 | T/G |
| 11 | 3225053 | A/T |
| 11 | 3274237 | C/G |
| 11 | 3314886 | T/C |
| 11 | 3332652 | C/T |
| 11 | 3371909 | T/C |
| 11 | 3395885 | C/T |
| 11 | 3412287 | C/T |
| 11 | 3423910 | G/A |
| 11 | 3425454 | A/G |
| 11 | 3467691 | A/G |
| 11 | 3486364 | A/G |
| 11 | 3528509 | C/T |
| 11 | 3537697 | C/A |
| 11 | 3559928 | T/A |
| 11 | 3574657 | A/C |
| 11 | 3608909 | A/C |
| 11 | 3638851 | T/C |
| 11 | 3653422 | A/G |
| 11 | 3661178 | G/A |
| 11 | 3664667 | G/A |
| 11 | 3717104 | G/A |
| 11 | 3728268 | C/G |
| 11 | 3730656 | C/A |
| 11 | 3787549 | G/T |
| 11 | 3831412 | G/T |
| 11 | 3848366 | C/T |
| 11 | 3874760 | A/G |
| 11 | 3894652 | T/A |
| 11 | 3907379 | T/A |
| 11 | 3913197 | A/C |
| 11 | 3936478 | C/T |
| 11 | 3961368 | A/G |
| 11 | 3995343 | G/A |
| 11 | 3999087 | A/G |
| 11 | 4026024 | A/T |
| 11 | 4071505 | C/T |
| 11 | 4107570 | C/A |
| 11 | 4117331 | C/T |
| 11 | 4134105 | C/G |
| 11 | 4146158 | G/A |
| 11 | 4191052 | C/T |
| 11 | 4198028 | G/A |
| 11 | 4217149 | A/T |
| 11 | 4224073 | G/T |
| 11 | 4240646 | A/G |
| 11 | 4262408 | G/A |
| 11 | 4287211 | T/C |
| 11 | 4296006 | A/C |
| 11 | 4341825 | T/C |
| 11 | 4348503 | T/C |
| 11 | 4367737 | T/C |
| 11 | 4391613 | C/T |
| 11 | 4428207 | T/G |
| 11 | 4438689 | C/T |
| 11 | 4457023 | G/A |
| 11 | 4481099 | G/T |
| 11 | 4492667 | G/A |
| 11 | 4502647 | G/A |
| 11 | 4519763 | G/A |
| 11 | 4537156 | T/C |
| 11 | 4574688 | T/C |
| 11 | 4613510 | C/T |
| 11 | 4616489 | C/T |
| 11 | 4630643 | C/T |
| 11 | 4658878 | G/C |
| 11 | 4674609 | C/T |
| 11 | 4686545 | C/T |
| 11 | 4691092 | T/A |
| 11 | 4734834 | T/C |
| 11 | 4745624 | C/T |
| 11 | 4765398 | C/T |
| 11 | 4792038 | T/C |
| 11 | 4812328 | C/T |
| 11 | 4832280 | C/T |
| 11 | 4842256 | G/A |
| 11 | 4895657 | G/A |
| 11 | 4935783 | A/G |
| 11 | 4957667 | G/A |
| 11 | 4967360 | T/G |
| 11 | 5004675 | C/T |
| 11 | 5034854 | C/T |
| 11 | 5049852 | C/T |
| 11 | 5054620 | G/T |
| 11 | 5077978 | G/A |
| 11 | 5085031 | A/G |
| 11 | 5104623 | G/A |
| 11 | 5114153 | C/T |
| 11 | 5135031 | G/A |
| 11 | 5164483 | C/T |
| 11 | 5191139 | G/T |
| 11 | 5237020 | G/A |
| 11 | 5260430 | G/A |
| 11 | 5278868 | C/T |
| 11 | 5280081 | G/A |
| 11 | 5299181 | C/T |
| 11 | 5335439 | G/C |
| 11 | 5346430 | G/A |
| 11 | 5352613 | G/A |
| 11 | 5373888 | C/T |
| 11 | 5410062 | A/G |
| 11 | 5414468 | G/A |
| 11 | 5442481 | C/T |
| 11 | 5462280 | G/A |
| 11 | 5466810 | G/A |
| 11 | 5513783 | A/T |
| 11 | 5549051 | C/T |
| 11 | 5551459 | A/G |
| 11 | 5607573 | C/T |
| 11 | 5621441 | C/T |
| 11 | 5638468 | C/T |
| 11 | 5689170 | T/C |
| 11 | 5725058 | G/A |
| 11 | 5740806 | C/T |
| 11 | 5767401 | A/T |
| 11 | 5780306 | G/A |
| 11 | 5806309 | T/A |
| 11 | 5810372 | G/A |
| 11 | 5823703 | C/T |
| 11 | 5865068 | C/T |
| 11 | 5892877 | C/T |
| 11 | 5905909 | A/T |
| 11 | 5916546 | C/T |
| 11 | 5945111 | G/C |
| 11 | 5978059 | T/A |
| 11 | 5980000 | C/G |
| 11 | 6002341 | G/A |
| 11 | 6015565 | T/C |
| 11 | 6050591 | T/C |
| 11 | 6069104 | A/G |
| 11 | 6117997 | G/C |
| 11 | 6135917 | G/C |
| 11 | 6145563 | G/A |
| 11 | 6151360 | C/T |
| 11 | 6182875 | G/A |
| 11 | 6192292 | G/T |
| 11 | 6260771 | G/A |
| 11 | 6268855 | G/A |
| 11 | 6289137 | G/A |
| 11 | 6297616 | T/C |
| 11 | 6320594 | A/G |
| 11 | 6322910 | A/G |
| 11 | 6338750 | A/G |
| 11 | 6364470 | C/T |
| 11 | 6390265 | C/T |
| 11 | 6402365 | C/T |
| 11 | 6429026 | G/T |
| 11 | 6459215 | A/G |
| 11 | 6549826 | A/G |
| 11 | 6585054 | T/C |
| 11 | 6595455 | A/T |
| 11 | 6606361 | G/A |
| 11 | 6658600 | G/A |
| 11 | 6665942 | C/T |
| 11 | 6668672 | T/C |
| 11 | 6739325 | C/T |
| 11 | 6756528 | G/C |
| 11 | 6771828 | A/G |
| 11 | 6786822 | A/C |
| 11 | 6823854 | C/T |
| 11 | 6853677 | G/A |
| 11 | 6872013 | A/G |
| 11 | 6882202 | T/A |
| 11 | 6916849 | C/A |
| 11 | 6949260 | G/T |
| 11 | 6959780 | T/C |
| 11 | 7019336 | C/T |
| 11 | 7066722 | A/T |
| 11 | 7073763 | G/A |
| 11 | 7100655 | T/C |
| 11 | 7106609 | C/T |
| 11 | 7114849 | G/A |
| 11 | 7116437 | C/T |
| 11 | 7182278 | G/A |
| 11 | 7201742 | C/T |
| 11 | 7225291 | G/A |
| 11 | 7259392 | T/C |
| 11 | 7273072 | G/T |
| 11 | 7290692 | C/A |
| 11 | 7296744 | C/T |
| 11 | 7323307 | G/T |
| 11 | 7328476 | G/A |
| 11 | 7358039 | G/A |
| 11 | 7391648 | T/C |
| 11 | 7400802 | C/A |
| 11 | 7426889 | C/T |
| 11 | 7464284 | G/A |
| 11 | 7497511 | G/A |
| 11 | 7514575 | C/T |
| 11 | 7529485 | T/A |
| 11 | 7539735 | G/A |
| 11 | 7547457 | C/T |
| 11 | 7575907 | A/G |
| 11 | 7614528 | G/T |
| 11 | 7624098 | T/C |
| 11 | 7627917 | G/A |
| 11 | 7677547 | A/G |
| 11 | 7692882 | G/A |
| 11 | 7731515 | C/G |
| 11 | 7787528 | G/A |
| 11 | 7815979 | G/A |
| 11 | 7819944 | C/G |
| 11 | 7835644 | G/A |
| 11 | 7885743 | A/C |
| 11 | 7888921 | G/T |
| 11 | 7899928 | A/T |
| 11 | 7916034 | T/C |
| 11 | 7942752 | C/A |
| 11 | 7975391 | A/G |
| 11 | 7985294 | T/C |
| 11 | 7991569 | T/C |
| 11 | 8031360 | T/C |
| 11 | 8041715 | A/G |
| 11 | 8048832 | G/A |
| 11 | 8070421 | A/G |
| 11 | 8082449 | T/C |
| 11 | 8128973 | A/G |
| 11 | 8139059 | C/T |
| 11 | 8141641 | C/T |
| 11 | 8177380 | G/A |
| 11 | 8178612 | A/G |
| 11 | 8197410 | G/A |
| 11 | 8239970 | T/G |
| 11 | 8246101 | A/G |
| 11 | 8271224 | G/A |
| 11 | 8277330 | C/T |
| 11 | 8288844 | C/G |
| 11 | 8311842 | G/A |
| 11 | 8353452 | A/G |
| 11 | 8355893 | G/A |
| 11 | 8378291 | T/C |
| 11 | 8412868 | G/A |
| 11 | 8429383 | G/A |
| 11 | 8434507 | C/T |
| 11 | 8442900 | C/T |
| 11 | 8461463 | G/A |
| 11 | 8519935 | A/G |
| 11 | 8560330 | T/C |
| 11 | 8561791 | T/C |
| 11 | 8580938 | G/C |
| 11 | 8597529 | A/G |
| 11 | 8613998 | T/C |
| 11 | 8639271 | C/T |
| 11 | 8651096 | G/A |
| 11 | 8665856 | T/C |
| 11 | 8692040 | G/C |
| 11 | 8694895 | C/T |
| 11 | 8711881 | G/A |
| 11 | 8729507 | T/C |
| 11 | 8755103 | G/A |
| 11 | 8756541 | C/T |
| 11 | 8770474 | C/T |
| 11 | 8780431 | C/T |
| 11 | 8796799 | G/A |
| 11 | 8822991 | C/T |
| 11 | 8879545 | T/C |
| 11 | 8882114 | C/T |
| 11 | 8892987 | T/C |
| 11 | 8915813 | G/A |
| 11 | 8919626 | G/T |
| 11 | 8940362 | G/T |
| 11 | 8945956 | C/A |
| 11 | 8989922 | C/A |
| 11 | 9021224 | C/T |
| 11 | 9027317 | C/T |
| 11 | 9053891 | A/G |
| 11 | 9060209 | T/G |
| 11 | 9111120 | A/G |
| 11 | 9123904 | A/G |
| 11 | 9156648 | A/T |
| 11 | 9198839 | T/C |
| 11 | 9211495 | G/A |
| 11 | 9252629 | C/T |
| 11 | 9269831 | G/T |
| 11 | 9287727 | T/C |
| 11 | 9307095 | T/C |
| 11 | 9316350 | T/C |
| 11 | 9355487 | T/C |
| 11 | 9397368 | T/C |
| 11 | 9402833 | A/G |
| 11 | 9422353 | G/A |
| 11 | 9455797 | T/C |
| 11 | 9511090 | G/T |
| 11 | 9540639 | C/T |
| 11 | 9541692 | C/T |
| 11 | 9593381 | G/A |
| 11 | 9620872 | G/T |
| 11 | 9652952 | C/T |
| 11 | 9676019 | T/G |
| 11 | 9703557 | C/T |
| 11 | 9745380 | T/G |
| 11 | 9754591 | G/A |
| 11 | 9762439 | T/A |
| 11 | 9815841 | T/C |
| 11 | 9842871 | A/T |
| 11 | 9887725 | T/A |
| 11 | 9916251 | C/T |
| 11 | 9949956 | T/C |
| 11 | 9951502 | G/A |
| 11 | 9973316 | A/C |
| 11 | 9984743 | G/A |
| 11 | 10036983 | C/T |
| 11 | 10068557 | C/T |
| 11 | 10105060 | C/T |
| 11 | 10135493 | T/C |
| 11 | 10150463 | A/T |
| 11 | 10157641 | C/T |
| 11 | 10184437 | A/G |
| 11 | 10212472 | C/T |
| 11 | 10278575 | T/G |
| 11 | 10316860 | A/G |
| 11 | 10330460 | C/T |
| 11 | 10338139 | A/T |
| 11 | 10362304 | C/T |
| 11 | 10365728 | C/T |
| 11 | 10399661 | C/T |
| 11 | 10407164 | G/C |
| 11 | 10424461 | C/T |
| 11 | 10437081 | A/T |
| 11 | 10441374 | A/G |
| 11 | 10491904 | G/T |
| 11 | 10496350 | T/C |
| 11 | 10520470 | T/C |
| 11 | 10538286 | T/C |
| 11 | 10546201 | C/T |
| 11 | 10578572 | C/T |
| 11 | 10593618 | T/C |
| 11 | 10607548 | T/C |
| 11 | 10640951 | A/C |
| 11 | 10644175 | T/C |
| 11 | 10650515 | A/G |
| 11 | 10670601 | C/T |
| 11 | 10683979 | A/G |
| 11 | 10706090 | T/C |
| 11 | 10732503 | G/T |
| 11 | 10751253 | T/C |
| 11 | 10782345 | T/G |
| 11 | 10811389 | A/G |
| 11 | 10845727 | G/T |
| 11 | 10870173 | C/T |
| 11 | 10891246 | G/A |
| 11 | 10912960 | C/G |
| 11 | 10929590 | G/A |
| 11 | 10955794 | G/C |
| 11 | 10979347 | T/C |
| 11 | 11038522 | G/A |
| 11 | 11089947 | C/T |
| 11 | 11092708 | C/T |
| 11 | 11105300 | C/T |
| 11 | 11132698 | A/T |
| 11 | 11152188 | T/C |
| 11 | 11205618 | G/T |
| 11 | 11234964 | C/T |
| 11 | 11236551 | G/A |
| 11 | 11250961 | G/A |
| 11 | 11301222 | T/C |
| 11 | 11342743 | T/G |
| 11 | 11382477 | T/A |
| 11 | 11389269 | T/C |
| 11 | 11400675 | G/A |
| 11 | 11407272 | C/T |
| 11 | 11434729 | A/G |
| 11 | 11446829 | C/G |
| 11 | 11464243 | G/A |
| 11 | 11502987 | T/C |
| 11 | 11510519 | A/G |
| 11 | 11523914 | T/G |
| 11 | 11526816 | T/C |
| 11 | 11552814 | T/C |
| 11 | 11571173 | T/C |
| 11 | 11589115 | G/A |
| 11 | 11601834 | T/C |
| 11 | 11617191 | C/T |
| 11 | 11624819 | A/G |
| 11 | 11668142 | G/A |
| 11 | 11675391 | G/A |
| 11 | 11679185 | T/C |
| 11 | 11701175 | G/A |
| 11 | 11730410 | C/T |
| 11 | 11746000 | G/A |
| 11 | 11761851 | A/G |
| 11 | 11767738 | G/T |
| 11 | 11797091 | A/T |
| 11 | 11815918 | C/T |
| 11 | 11826943 | T/C |
| 11 | 11839913 | T/C |
| 11 | 11863608 | A/G |
| 11 | 11892308 | C/A |
| 11 | 11896107 | T/C |
| 11 | 11912959 | T/C |
| 11 | 11949306 | T/C |
| 11 | 11979640 | A/C |
| 11 | 11988661 | A/G |
| 11 | 12013043 | G/A |
| 11 | 12015307 | C/T |
| 11 | 12043039 | G/A |
| 11 | 12081512 | G/A |
| 11 | 12118628 | G/A |
| 11 | 12123729 | G/C |
| 11 | 12125367 | C/G |
| 11 | 12152545 | C/G |
| 11 | 12160605 | C/T |
| 11 | 12196588 | G/A |
| 11 | 12219494 | G/A |
| 11 | 12239233 | G/A |
| 11 | 12241307 | A/G |
| 11 | 12251124 | G/T |
| 11 | 12290573 | T/C |
| 11 | 12299093 | T/A |
| 11 | 12300156 | T/A |
| 11 | 12323778 | A/G |
| 11 | 12359516 | A/C |
| 11 | 12361072 | G/A |
| 11 | 12369291 | T/C |
| 11 | 12392662 | T/C |
| 11 | 12437427 | C/T |
| 11 | 12459383 | T/C |
| 11 | 12477107 | T/C |
| 11 | 12502182 | A/G |
| 11 | 12504354 | T/C |
| 11 | 12526893 | G/T |
| 11 | 12534106 | C/G |
| 11 | 12554354 | G/A |
| 11 | 12599988 | C/T |
| 11 | 12623679 | A/T |
| 11 | 12636520 | G/C |
| 11 | 12659743 | C/G |
| 11 | 12663679 | G/A |
| 11 | 12668834 | T/G |
| 11 | 12706957 | T/G |
| 11 | 12735219 | T/C |
| 11 | 12764261 | T/G |
| 11 | 12788791 | A/G |
| 11 | 12848029 | A/G |
| 11 | 12879071 | C/T |
| 11 | 12882561 | A/G |
| 11 | 12925749 | G/A |
| 11 | 12938949 | G/A |
| 11 | 12967898 | G/C |
| 11 | 12990205 | C/T |
| 11 | 13012551 | A/G |
| 11 | 13025789 | G/A |
| 11 | 13049972 | G/T |
| 11 | 13063607 | G/A |
| 11 | 13083938 | C/T |
| 11 | 13093436 | A/G |
| 11 | 13122430 | T/C |
| 11 | 13134637 | A/G |
| 11 | 13160121 | G/A |
| 11 | 13173837 | A/C |
| 11 | 13181234 | A/G |
| 11 | 13210209 | G/A |
| 11 | 13226303 | T/G |
| 11 | 13236105 | T/C |
| 11 | 13237521 | A/G |
| 11 | 13285879 | C/T |
| 11 | 13289406 | T/C |
| 11 | 13291046 | A/C |
| 11 | 13332590 | T/C |
| 11 | 13350222 | T/C |
| 11 | 13401159 | G/A |
| 11 | 13407013 | T/A |
| 11 | 13410325 | T/C |
| 11 | 13453839 | T/A |
| 11 | 13460389 | A/G |
| 11 | 13475667 | A/G |
| 11 | 13477341 | T/A |
| 11 | 13501936 | C/T |
| 11 | 13576470 | A/G |
| 11 | 13579176 | G/A |
| 11 | 13617425 | C/A |
| 11 | 13621228 | G/A |
| 11 | 13637359 | A/G |
| 11 | 13668395 | C/T |
| 11 | 13678377 | C/A |
| 11 | 13697559 | G/A |
| 11 | 13732792 | T/G |
| 11 | 13737205 | G/T |
| 11 | 13740202 | G/A |
| 11 | 13752684 | C/T |
| 11 | 13786820 | C/G |
| 11 | 13795334 | T/A |
| 11 | 13801466 | T/C |
| 11 | 13804429 | A/T |
| 11 | 13836454 | G/A |
| 11 | 13875522 | C/T |
| 11 | 13882989 | A/G |
| 11 | 13893155 | T/C |
| 11 | 13911669 | C/T |
| 11 | 13946345 | T/C |
| 11 | 13972213 | G/C |
| 11 | 13993419 | A/G |
| 11 | 14022555 | C/T |
| 11 | 14062754 | C/T |
| 11 | 14062755 | C/T |
| 11 | 14062756 | C/T |
| 11 | 14062757 | C/T |
| 11 | 14128321 | C/T |
| 11 | 14156360 | G/A |
| 11 | 14184830 | C/T |
| 11 | 14211036 | G/A |
| 11 | 14255165 | G/T |
| 11 | 14284965 | A/G |
| 11 | 14343711 | G/A |
| 11 | 14362161 | C/T |
| 11 | 14390666 | G/A |
| 11 | 14400324 | G/A |
| 11 | 14406378 | T/A |
| 11 | 14443899 | C/T |
| 11 | 14445782 | C/T |
| 11 | 14473390 | G/A |
| 11 | 14505548 | C/T |
| 11 | 14530946 | A/G |
| 11 | 14542223 | A/G |
| 11 | 14557902 | T/G |
| 11 | 14572396 | G/T |
| 11 | 14617479 | C/T |
| 11 | 14648170 | T/A |
| 11 | 14687783 | C/T |
| 11 | 14702306 | C/T |
| 11 | 14750332 | G/A |
| 11 | 14816413 | A/G |
| 11 | 14854498 | T/C |
| 11 | 14872231 | C/T |
| 11 | 14882321 | A/C |
| 11 | 14933744 | G/A |
| 11 | 14949821 | A/G |
| 11 | 14974217 | A/T |
| 11 | 14983205 | G/A |
| 11 | 15006671 | G/A |
| 11 | 15047026 | C/T |
| 11 | 15060438 | T/G |
| 11 | 15082408 | T/C |
| 11 | 15094337 | G/A |
| 11 | 15127426 | A/G |
| 11 | 15164405 | G/A |
| 11 | 15191372 | A/T |
| 11 | 15199321 | C/T |
| 11 | 15226175 | C/T |
| 11 | 15254904 | C/A |
| 11 | 15292489 | G/A |
| 11 | 15343018 | A/G |
| 11 | 15362243 | C/T |
| 11 | 15367156 | C/T |
| 11 | 15418627 | G/A |
| 11 | 15441074 | C/T |
| 11 | 15453018 | C/T |
| 11 | 15476240 | C/T |
| 11 | 15514391 | A/G |
| 11 | 15540790 | A/G |
| 11 | 15566416 | C/G |
| 11 | 15570498 | G/A |
| 11 | 15579894 | G/C |
| 11 | 15600174 | A/G |
| 11 | 15603729 | C/T |
| 11 | 15632304 | A/G |
| 11 | 15644790 | A/G |
| 11 | 15674594 | A/G |
| 11 | 15677761 | G/A |
| 11 | 15702309 | C/A |
| 11 | 15709398 | T/G |
| 11 | 15733190 | A/C |
| 11 | 15789917 | C/T |
| 11 | 15815796 | T/C |
| 11 | 15817029 | C/A |
| 11 | 15848202 | T/C |
| 11 | 15855791 | C/G |
| 11 | 15888710 | C/T |
| 11 | 15898479 | G/T |
| 11 | 15923343 | A/G |
| 11 | 15934312 | A/G |
| 11 | 15961188 | T/C |
| 11 | 15998822 | T/G |
| 11 | 16000150 | C/G |
| 11 | 16028998 | A/G |
| 11 | 16039413 | C/A |
| 11 | 16051311 | C/T |
| 11 | 16079992 | G/T |
| 11 | 16088238 | G/A |
| 11 | 16092400 | G/A |
| 11 | 16126316 | G/A |
| 11 | 16128926 | G/A |
| 11 | 16155010 | C/A |
| 11 | 16157729 | C/G |
| 11 | 16192681 | G/A |
| 11 | 16203705 | C/G |
| 11 | 16231375 | C/A |
| 11 | 16256352 | G/A |
| 11 | 16267351 | A/C |
| 11 | 16303341 | C/A |
| 11 | 16319989 | C/A |
| 11 | 16328272 | C/T |
| 11 | 16368295 | T/C |
| 11 | 16391054 | G/C |
| 11 | 16401499 | T/C |
| 11 | 16411879 | A/G |
| 11 | 16434275 | C/T |
| 11 | 16443906 | C/G |
| 11 | 16511579 | C/G |
| 11 | 16553185 | A/G |
| 11 | 16555844 | A/C |
| 11 | 16566216 | G/T |
| 11 | 16567748 | G/C |
| 11 | 16600101 | G/A |
| 11 | 16651438 | T/C |
| 11 | 16663956 | C/T |
| 11 | 16729425 | T/C |
| 11 | 16768386 | G/T |
| 11 | 16777494 | C/T |
| 11 | 16779628 | G/A |
| 11 | 16811597 | T/C |
| 11 | 16845388 | G/A |
| 11 | 16867814 | G/A |
| 11 | 16889843 | A/G |
| 11 | 16933326 | G/A |
| 11 | 16942791 | G/A |
| 11 | 16964518 | C/T |
| 11 | 17002031 | C/T |
| 11 | 17019529 | G/A |
| 11 | 17027982 | A/G |
| 11 | 17040478 | G/A |
| 11 | 17063904 | C/G |
| 11 | 17087305 | G/A |
| 11 | 17153730 | T/C |
| 11 | 17162559 | G/A |
| 11 | 17176971 | T/C |
| 11 | 17194652 | C/T |
| 11 | 17195807 | C/T |
| 11 | 17221560 | T/C |
| 11 | 17258815 | C/T |
| 11 | 17293645 | G/A |
| 11 | 17322999 | G/C |
| 11 | 17348737 | C/T |
| 11 | 17398995 | G/A |
| 11 | 17450294 | A/T |
| 11 | 17497233 | G/A |
| 11 | 17530798 | T/C |
| 11 | 17564169 | G/A |
| 11 | 17581730 | A/T |
| 11 | 17591090 | G/A |
| 11 | 17615079 | G/T |
| 11 | 17661166 | G/A |
| 11 | 17686686 | G/A |
| 11 | 17726963 | G/T |
| 11 | 17745501 | G/A |
| 11 | 17757823 | G/A |
| 11 | 17764163 | A/G |
| 11 | 17800749 | T/C |
| 11 | 17815635 | C/T |
| 11 | 17831328 | G/T |
| 11 | 17848884 | C/T |
| 11 | 17896453 | A/G |
| 11 | 17934980 | G/A |
| 11 | 17939858 | G/T |
| 11 | 17951286 | G/A |
| 11 | 17974279 | C/T |
| 11 | 17998611 | G/A |
| 11 | 18009650 | A/G |
| 11 | 18078163 | C/T |
| 11 | 18095049 | G/C |
| 11 | 18113445 | C/T |
| 11 | 18123162 | T/G |
| 11 | 18147841 | A/T |
| 11 | 18161022 | G/T |
| 11 | 18163297 | C/T |
| 11 | 18185477 | G/A |
| 11 | 18224326 | G/A |
| 11 | 18226643 | C/G |
| 11 | 18246610 | G/A |
| 11 | 18258534 | C/T |
| 11 | 18280642 | G/A |
| 11 | 18291625 | A/C |
| 11 | 18326542 | T/C |
| 11 | 18353278 | C/T |
| 11 | 18365433 | G/T |
| 11 | 18415807 | A/G |
| 11 | 18419213 | T/C |
| 11 | 18440723 | A/C |
| 11 | 18483288 | C/T |
| 11 | 18550568 | C/A |
| 11 | 18580494 | C/T |
| 11 | 18650017 | C/G |
| 11 | 18675934 | C/T |
| 11 | 18686443 | C/T |
| 11 | 18694334 | G/A |
| 11 | 18745110 | C/T |
| 11 | 18745111 | A/C |
| 11 | 18745112 | A/C |
| 11 | 18745113 | A/C |
| 11 | 18791353 | G/A |
| 11 | 18792921 | C/T |
| 11 | 18812013 | C/A |
| 11 | 18826329 | G/A |
| 11 | 18870405 | G/A |
| 11 | 18915204 | C/T |
| 11 | 18944266 | G/A |
| 11 | 19041491 | C/A |
| 11 | 19068462 | G/A |
| 11 | 19089661 | G/A |
| 11 | 19112359 | G/A |
| 11 | 19133066 | A/G |
| 11 | 19145483 | T/C |
| 11 | 19188327 | T/G |
| 11 | 19198355 | A/G |
| 11 | 19217092 | G/A |
| 11 | 19228502 | A/G |
| 11 | 19245724 | G/A |
| 11 | 19280442 | T/C |
| 11 | 19288820 | G/A |
| 11 | 19305691 | G/A |
| 11 | 19313747 | C/T |
| 11 | 19338046 | C/T |
| 11 | 19348612 | C/A |
| 11 | 19362666 | T/C |
| 11 | 19377793 | A/G |
| 11 | 19380917 | C/T |
| 11 | 19412798 | G/A |
| 11 | 19424029 | C/T |
| 11 | 19424030 | T/C |
| 11 | 19424031 | T/C |
| 11 | 19424032 | T/C |
| 11 | 19470485 | T/G |
| 11 | 19501284 | G/A |
| 11 | 19502992 | C/T |
| 11 | 19535056 | T/A |
| 11 | 19545195 | C/T |
| 11 | 19567448 | T/C |
| 11 | 19594106 | T/G |
| 11 | 19637502 | A/G |
| 11 | 19645502 | T/A |
| 11 | 19650468 | A/C |
| 11 | 19661120 | A/G |
| 11 | 19701724 | G/A |
| 11 | 19734365 | G/A |
| 11 | 19742573 | G/A |
| 11 | 19783202 | A/G |
| 11 | 19784216 | A/G |
| 11 | 19826840 | T/C |
| 11 | 19848360 | A/G |
| 11 | 19860572 | G/A |
| 11 | 19882233 | A/G |
| 11 | 19916841 | C/T |
| 11 | 19921711 | C/A |
| 11 | 19944104 | C/T |
| 11 | 19959353 | C/T |
| 11 | 19961263 | G/A |
| 11 | 19992195 | G/C |
| 11 | 20004476 | T/A |
| 11 | 20011994 | G/A |
| 11 | 20031131 | T/G |
| 11 | 20040426 | C/G |
| 11 | 20047261 | C/T |
| 11 | 20099246 | T/C |
| 11 | 20117933 | G/A |
| 11 | 20150493 | T/C |
| 11 | 20169908 | G/T |
| 11 | 20211543 | C/T |
| 11 | 20224345 | C/T |
| 11 | 20249370 | G/A |
| 11 | 20264874 | C/T |
| 11 | 20289735 | G/A |
| 11 | 20333563 | A/T |
| 11 | 20385021 | T/G |
| 11 | 20410110 | C/T |
| 11 | 20430609 | C/T |
| 11 | 20449158 | C/T |
| 11 | 20463228 | G/A |
| 11 | 20468152 | C/T |
| 11 | 20492695 | A/G |
| 11 | 20533010 | G/A |
| 11 | 20602734 | A/G |
| 11 | 20620949 | A/C |
| 11 | 20655713 | C/T |
| 11 | 20660390 | G/C |
| 11 | 20677342 | A/T |
| 11 | 20678412 | A/G |
| 11 | 20726299 | G/C |
| 11 | 20750281 | C/T |
| 11 | 20760947 | C/G |
| 11 | 20788742 | C/T |
| 11 | 20792228 | C/G |
| 11 | 20797277 | G/C |
| 11 | 20835317 | A/T |
| 11 | 20849975 | G/A |
| 11 | 20875665 | T/A |
| 11 | 20881791 | C/T |
| 11 | 20888753 | C/T |
| 11 | 20921639 | G/A |
| 11 | 20944785 | C/A |
| 11 | 20981619 | T/A |
| 11 | 21020565 | T/G |
| 11 | 21050430 | G/A |
| 11 | 21089284 | C/T |
| 11 | 21099192 | T/C |
| 11 | 21104093 | C/T |
| 11 | 21135204 | T/C |
| 11 | 21148306 | C/T |
| 11 | 21150288 | T/C |
| 11 | 21179901 | G/C |
| 11 | 21184288 | A/G |
| 11 | 21186187 | C/T |
| 11 | 21211276 | G/T |
| 11 | 21215897 | C/T |
| 11 | 21252909 | G/C |
| 11 | 21262072 | C/T |
| 11 | 21292940 | C/T |
| 11 | 21296554 | T/C |
| 11 | 21315247 | T/C |
| 11 | 21324713 | C/T |
| 11 | 21361791 | T/A |
| 11 | 21403905 | T/C |
| 11 | 21431288 | C/T |
| 11 | 21445724 | C/G |
| 11 | 21466958 | A/G |
| 11 | 21485938 | A/T |
| 11 | 21498071 | A/C |
| 11 | 21538141 | C/T |
| 11 | 21557989 | T/G |
| 11 | 21560058 | G/A |
| 11 | 21583088 | G/A |
| 11 | 21593785 | C/G |
| 11 | 21600511 | C/A |
| 11 | 21603156 | T/C |
| 11 | 21630143 | A/G |
| 11 | 21655742 | C/A |
| 11 | 21660214 | A/C |
| 11 | 21662944 | A/G |
| 11 | 21694679 | A/C |
| 11 | 21700381 | A/G |
| 11 | 21731930 | T/C |
| 11 | 21760087 | A/T |
| 11 | 21792783 | C/T |
| 11 | 21801139 | T/G |
| 11 | 21810936 | A/C |
| 11 | 21820848 | G/A |
| 11 | 21864864 | C/T |
| 11 | 21884567 | G/A |
| 11 | 21888990 | T/G |
| 11 | 21902730 | T/C |
| 11 | 21907157 | A/G |
| 11 | 21935685 | T/C |
| 11 | 21977754 | G/T |
| 11 | 21985589 | C/G |
| 11 | 21997843 | C/A |
| 11 | 22025428 | T/C |
| 11 | 22055662 | C/T |
| 11 | 22074364 | C/T |
| 11 | 22093529 | G/A |
| 11 | 22122794 | G/C |
| 11 | 22126711 | A/G |
| 11 | 22152500 | C/T |
| 11 | 22154172 | G/C |
| 11 | 22176181 | A/C |
| 11 | 22177729 | T/C |
| 11 | 22217872 | T/G |
| 11 | 22241244 | T/C |
| 11 | 22243613 | A/G |
| 11 | 22284924 | T/C |
| 11 | 22286446 | C/T |
| 11 | 22311210 | C/T |
| 11 | 22331773 | A/C |
| 11 | 22344311 | G/C |
| 11 | 22369775 | A/C |
| 11 | 22380835 | G/A |
| 11 | 22437324 | G/A |
| 11 | 22449895 | C/T |
| 11 | 22498896 | G/A |
| 11 | 22500249 | A/G |
| 11 | 22505103 | T/A |
| 11 | 22531736 | T/A |
| 11 | 22532692 | T/C |
| 11 | 22560054 | C/T |
| 11 | 22562645 | C/A |
| 11 | 22600893 | G/T |
| 11 | 22644555 | C/T |
| 11 | 22677473 | A/G |
| 11 | 22678967 | C/T |
| 11 | 22682282 | A/G |
| 11 | 22700468 | T/C |
| 11 | 22712740 | A/G |
| 11 | 22769226 | T/C |
| 11 | 22771066 | A/G |
| 11 | 22802848 | G/A |
| 11 | 22821089 | T/C |
| 11 | 22855050 | C/T |
| 11 | 22864668 | C/T |
| 11 | 22887083 | C/T |
| 11 | 22892860 | A/T |
| 11 | 22894536 | T/A |
| 11 | 22938067 | G/T |
| 11 | 22951607 | A/T |
| 11 | 22960380 | T/C |
| 11 | 22981437 | G/A |
| 11 | 22984999 | C/T |
| 11 | 23030256 | A/G |
| 11 | 23051081 | G/A |
| 11 | 23054581 | C/T |
| 11 | 23078817 | T/C |
| 11 | 23143458 | G/T |
| 11 | 23160099 | G/A |
| 11 | 23170803 | C/T |
| 11 | 23217653 | G/A |
| 11 | 23228842 | T/C |
| 11 | 23256290 | A/C |
| 11 | 23274611 | C/A |
| 11 | 23309284 | G/C |
| 11 | 23314098 | A/G |
| 11 | 23318644 | T/C |
| 11 | 23350675 | C/A |
| 11 | 23370503 | G/T |
| 11 | 23410212 | C/T |
| 11 | 23435280 | C/T |
| 11 | 23437614 | C/T |
| 11 | 23481769 | G/T |
| 11 | 23484205 | G/A |
| 11 | 23527567 | G/C |
| 11 | 23535543 | A/T |
| 11 | 23563476 | T/C |
| 11 | 23616426 | T/C |
| 11 | 23620835 | G/A |
| 11 | 23646118 | G/T |
| 11 | 23684092 | T/A |
| 11 | 23693418 | A/C |
| 11 | 23700218 | T/C |
| 11 | 23732561 | G/T |
| 11 | 23734730 | T/G |
| 11 | 23761331 | C/G |
| 11 | 23796806 | C/A |
| 11 | 23826489 | A/G |
| 11 | 23836969 | A/T |
| 11 | 23854247 | A/G |
| 11 | 23877270 | A/G |
| 11 | 23880510 | A/G |
| 11 | 23881510 | T/C |
| 11 | 23927263 | G/A |
| 11 | 23935391 | T/G |
| 11 | 23943533 | G/C |
| 11 | 23975416 | A/C |
| 11 | 24024551 | C/T |
| 11 | 24026933 | A/C |
| 11 | 24034187 | G/A |
| 11 | 24038167 | C/T |
| 11 | 24068508 | G/A |
| 11 | 24115145 | C/T |
| 11 | 24119336 | G/A |
| 11 | 24135356 | C/G |
| 11 | 24136800 | T/C |
| 11 | 24162124 | C/T |
| 11 | 24169923 | A/T |
| 11 | 24182459 | C/G |
| 11 | 24184147 | T/C |
| 11 | 24234124 | T/C |
| 11 | 24235485 | C/T |
| 11 | 24240168 | A/G |
| 11 | 24266850 | G/A |
| 11 | 24270004 | C/T |
| 11 | 24282414 | C/T |
| 11 | 24375330 | G/A |
| 11 | 24391237 | G/T |
| 11 | 24393027 | G/C |
| 11 | 24422175 | C/G |
| 11 | 24426753 | G/A |
| 11 | 24476598 | C/A |
| 11 | 24478451 | G/A |
| 11 | 24505640 | T/G |
| 11 | 24514083 | G/A |
| 11 | 24544177 | G/T |
| 11 | 24554381 | C/T |
| 11 | 24570531 | T/C |
| 11 | 24581734 | G/A |
| 11 | 24616851 | G/A |
| 11 | 24630239 | G/T |
| 11 | 24667407 | A/G |
| 11 | 24690238 | A/G |
| 11 | 24691577 | C/T |
| 11 | 24721907 | A/G |
| 11 | 24724469 | A/T |
| 11 | 24751358 | G/T |
| 11 | 24762231 | G/A |
| 11 | 24780811 | G/C |
| 11 | 24811542 | C/T |
| 11 | 24819594 | T/A |
| 11 | 24840596 | G/A |
| 11 | 24873402 | T/A |
| 11 | 24889859 | T/C |
| 11 | 24902181 | C/T |
| 11 | 24910503 | A/C |
| 11 | 24964167 | A/T |
| 11 | 25005456 | G/A |
| 11 | 25026121 | T/C |
| 11 | 25050216 | A/T |
| 11 | 25052182 | G/A |
| 11 | 25080258 | A/T |
| 11 | 25137839 | G/A |
| 11 | 25170591 | A/T |
| 11 | 25178552 | T/C |
| 11 | 25216211 | T/C |
| 11 | 25220338 | C/A |
| 11 | 25238687 | A/G |
| 11 | 25265997 | C/T |
| 11 | 25306135 | G/T |
| 11 | 25325766 | G/A |
| 11 | 25352302 | C/T |
| 11 | 25375294 | C/T |
| 11 | 25380032 | T/A |
| 11 | 25403232 | C/T |
| 11 | 25420589 | C/A |
| 11 | 25439042 | C/T |
| 11 | 25451790 | A/G |
| 11 | 25467226 | A/G |
| 11 | 25495404 | C/T |
| 11 | 25499539 | A/T |
| 11 | 25520630 | G/A |
| 11 | 25523590 | T/G |
| 11 | 25543147 | G/A |
| 11 | 25561077 | A/G |
| 11 | 25564241 | G/T |
| 11 | 25646989 | C/T |
| 11 | 25649137 | T/C |
| 11 | 25655321 | C/T |
| 11 | 25657056 | G/A |
| 11 | 25704492 | C/T |
| 11 | 25725097 | A/T |
| 11 | 25746098 | G/C |
| 11 | 25770264 | A/G |
| 11 | 25795636 | C/T |
| 11 | 25817666 | A/G |
| 11 | 25824495 | G/A |
| 11 | 25831903 | T/A |
| 11 | 25843791 | G/T |
| 11 | 25869441 | G/T |
| 11 | 25892518 | A/G |
| 11 | 25932672 | G/T |
| 11 | 25957034 | G/A |
| 11 | 25997387 | G/T |
| 11 | 26013266 | T/G |
| 11 | 26016096 | G/C |
| 11 | 26055300 | C/T |
| 11 | 26060547 | C/T |
| 11 | 26083007 | C/T |
| 11 | 26089910 | C/T |
| 11 | 26115178 | G/A |
| 11 | 26126743 | C/A |
| 11 | 26159704 | C/A |
| 11 | 26176701 | C/A |
| 11 | 26198077 | A/G |
| 11 | 26227706 | C/T |
| 11 | 26228950 | C/G |
| 11 | 26255927 | A/C |
| 11 | 26262977 | C/T |
| 11 | 26280423 | A/C |
| 11 | 26282402 | G/C |
| 11 | 26315202 | A/G |
| 11 | 26356458 | A/C |
| 11 | 26368720 | T/C |
| 11 | 26383248 | G/T |
| 11 | 26388269 | C/T |
| 11 | 26403372 | T/C |
| 11 | 26405211 | G/A |

### (3) the Detection Principle is as Follows.

Plant genomic DNA consists of four types of bases, namely A, C, G, and T. In the detection process of the SNP chip, bases A and T are labeled with dinitrophenol (DNP), which can bind to DNP-antibodies marked with red fluorescence, thus displaying red color; bases C and G are labeled with biotin, which can bind to streptavidin marked with green fluorescence, thus displaying green color. When SNP genotyping is A/T or G/C, only one color label is present, requiring two probes for detection; when SNP genotyping is not A/T or G/C, two color labels are present, and one probe can complete the detection.

### (4) the Detection Method is as Follows.

(1) DNA extraction is performed, including: extracting DNA from the mung bean samples to be tested using a DNA extraction kit. (2) DNA quality control is performed, including: analyzing DNA quality using agarose gel electrophoresis and precisely checking integrity with Agilent 2100 (a bioanalyzer purchased from Agilent Technologies, Inc.). The quality control standard includes a total amount of no less than 2 micrograms (g), a sample concentration of less than 50 nanograms per microliter (ng/μL), and no impurities. (3) Library construction is performed, including: randomly breaking the qualified sample DNA using an ultrasonic disrupter, recovering the required length of DNA fragments by electrophoresis, and adding adapters to their ends to form a library. (4) The sequencing library is constructed, including: amplifying the sample library using ligation-mediated PCR (LMPCR) and performing purification for subsequent probe hybridization experiments. (5) The hybridization capture library is constructed, including: taking 300 ng of the constructed sequencing library, followed by freeze-drying to obtain a freeze-dried library, adding mung bean 20K SNP target capture probes and hybridization reagents to the freeze-dried library, followed by denaturing and incubating at 60° C. for 16 hours for the hybridization reaction to obtain a hybridization product; and washing the hybridization product followed by performing 5 rounds of PCR to complete the construction of the hybridization capture library. (6) Quality control is performed on the hybridization capture library, including: accurately quantifying the effective concentration of the hybridization capture library using quantitative PCR (qPCR) to ensure the library quality. (7) Sequencing is performed, including: measuring the library concentration with a Qubit fluorometer (purchased from thermo fisher scientific Inc.), and checking the fragment size with agarose gel electrophoresis; where after the concentration and the fragment size are qualified, the sequencing library construction is completed; and using the sequencing library for high-throughput sequencing with a sequencer (purchased from MGI Tech Co., Ltd). (8) Analysis is performed, including: after obtaining the raw data, using the BWA to align the sequencing data with the reference genome, and using the GATK to perform standard SNP calling on the data to obtain the genotyping results of the genome to be tested.

### Embodiment 2: Mung Bean 20K Liquid-Phase Gene Chip Target Site Capture Efficiency

A total of 25 test samples are used, including 18 cultivated mung bean materials and 7 wild and closely related mung bean materials. The mung bean 20K liquid-phase gene chip is designed with an addition of 5% target sites based on preliminary evaluation. The actual capture rate is such that all target sites exceeding 20K could be normally captured, yielding results. The overall site capture rate exceeds 9600, meeting the designed capture efficiency. The detection can be standardized and promoted. The specific detection rate statistics are shown in Table 3.

TABLE 3
Mung bean 20K liquid-phase gene chip
target site capture data display

| Testing | Coverage depth- | Coverage depth- | Coverage depth- |
|---|---|---|---|
| sample-name | coverage_1x | coverage_5x | coverage_10x |
| SL1 | 0.999226 | 0.998888 | 0.998549393 |
| KPS1 | 0.999081 | 0.998356 | 0.998017504 |
| KPS2 | 0.985107 | 0.980223 | 0.977660655 |
| NM | 0.96359 | 0.954258 | 0.949180407 |
| G12 | 0.983947 | 0.975968 | 0.973308834 |
| G204 | 0.978047 | 0.971423 | 0.96890866 |
| ASG | 0.981722 | 0.974421 | 0.97108457 |
| G274 | 0.97945 | 0.962913 | 0.945457183 |
| G327 | 0.990571 | 0.975291 | 0.959818191 |
| JL7 | 0.984333 | 0.977274 | 0.97393743 |
| V2709 | 0.94918 | 0.933417 | 0.926986123 |
| V2817 | 0.969876 | 0.961462 | 0.95899618 |
| NJY | 0.975485 | 0.966829 | 0.96320294 |
| Xbz | 0.975727 | 0.966153 | 0.96252599 |
| NAU | 0.973986 | 0.964557 | 0.959818191 |
| CN60 | 0.984817 | 0.97945 | 0.97746724 |
| G250 | 0.984333 | 0.974179 | 0.97026256 |
| G393 | 0.983995 | 0.977371 | 0.974420966 |
| TC1966 | 0.878923 | 0.836903 | 0.824379866 |
| JP229327 | 0.816788 | 0.773367 | 0.758764083 |
| JP229281 | 0.828538 | 0.78149 | 0.76809632 |
| JP229290 | 0.831294 | 0.777719 | 0.762245539 |
| JP229287 | 0.837725 | 0.79295 | 0.780329771 |
| JP226873 | 0.799478 | 0.740003 | 0.696484696 |
| NI1135 | 0.668972 | 0.607272 | 0.589720033 |

### Embodiment 3: Analysis for Functional Gene Sites of the Green Core No. 1 Chip

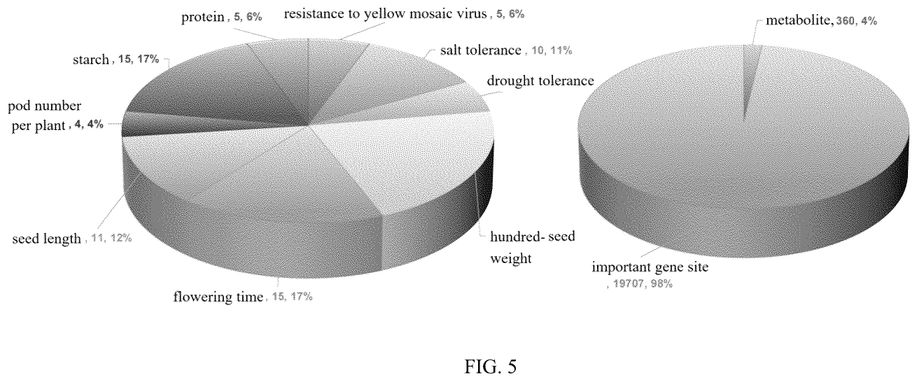

Experimental materials: the green core No. 1 chip; source of genotypic data: obtained from the chip. The green core No. 1 chip possesses sites related to agronomic traits and disease traits, as shown in FIG. 5. The sites related to functional genes include those that have been reported to be associated with the agronomic traits such as a hundred-seed weight, a seed length, starch, protein, and a number of pods per plant, and metabolites like flavonoids. The sites related to functional genes further include sites related to stress-resistant traits such as mung bean yellow mosaic virus disease, salt tolerance, and drought tolerance.

### Embodiment 4: The Application of the Mung Bean SNP Molecular Marker Combination in Polymorphism and Population Structure Analysis of Mung Bean Breeding Materials

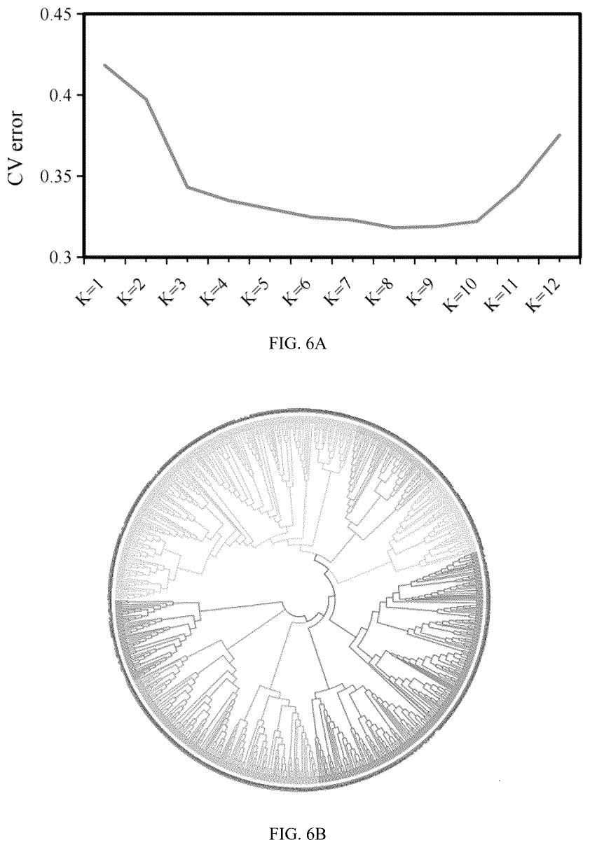

Using the developed mung bean 20K liquid-phase breeding chip, 892 mung bean MAGIC materials (a population derived from 8 parental lines) are tested to extract the genotyping of the target sites. The average detection rate of the samples is 99.10%, and the consistency rate for the same batch of materials is 99.62%, indicating that the detection rate of the target sites of this liquid-phase chip is high and the genotyping results are accurate and reliable. An ADMIXTURE software (a group structure analysis software) is used to estimate the ancestral components of individuals, and to divide the genotypes into five equally-sized parts for cross-validation to search for the optimal K value from 1 to 12 (FIG. 6A). The genetic distance matrix is then calculated using the Plink software, and clustering analysis is performed on the mung bean breeding materials to construct a phylogenetic tree that could determine the kinship, evolutionary relationships, and compositional structure of different materials. The clustering analysis is performed on 892 mung bean materials from different populations using the disclosure, which reveals that the mung bean breeding materials are divided into eight subgroups, consistent with the actual grouping, as shown in the analysis results in FIG. 6B, indicating that the selected 20,526 SNP sites have high representativeness.

### Embodiment 5: Association Analysis for Resistance to Yellow Mosaic Disease of the Green Core No. 1 Chip

Experimental materials: population analysis of 892 MAGIC materials; the source of genotypic data: obtained from green core No. 1 chip detection; and an analysis model: a mixed linear model (MLM).

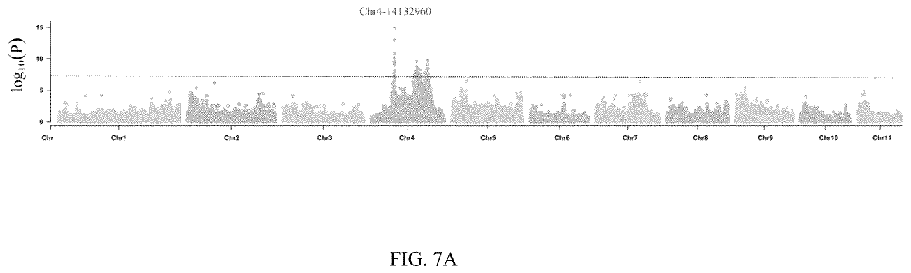

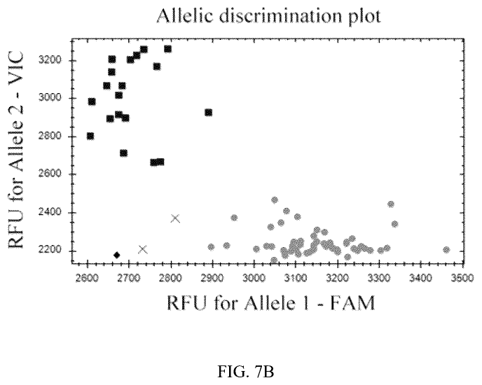

Experimental results: Using the MLM for association analysis of resistance to yellow mosaic disease in the tested population, the results show that at the level of P<1×10⁻⁷, one significantly associated site with yellow mosaic disease is identified on four chromosomes (FIG. 7A). Near this site Chr4-14132960, there are more than ten genes, including EVM0033654, EVM0003495, EVM0024933, EVM0009281, EVM0009509, EVM0029763, EVM0004327, EVM0008202, EVM0009824, EVM0009117, and EVM0025596, etc.

| Gene ID | Chr | Start | End |
|---|---|---|---|
| EVM0033654 | LG04 | 14110795 | 14115633 |
| EVM0007493 | LG04 | 14170656 | 14177955 |
| EVM0024933 | LG04 | 14170656 | 14177955 |
| EVM0009281 | LG04 | 14210403 | 14212432 |
| EVM0009509 | LG04 | 14237633 | 14238400 |
| EVM0029763 | LG04 | 14263554 | 14265781 |
| EVM0004327 | LG04 | 14269049 | 14272530 |
| EVM0008202 | LG04 | 14274475 | 14280997 |
| EVM0009824 | LG04 | 14313769 | 14318275 |
| EVM0009117 | LG04 | 14321154 | 14324994 |
| EVM0025596 | LG04 | 14326929 | 14332867 |

The Arabidopsis homolog gene AT5G24320 of the EVM0003495 is a gene with a typical disease resistance gene structural feature, possessing a WD40 repeat structural domain.

The EVM0030586 (pathogenesis-related gene) is related to proteins involved in the pathogenesis mechanism against environmental stress. At the same time, the site Chr4-14132960 can be developed into a KASP molecular marker for the identification of varieties resistant to the yellow mosaic disease. The marker testing is performed on 76 materials, and the KASP molecular marker mymv-1 achieves an accuracy rate of 98.61% in distinguishing between resistant and susceptible varieties (FIG. 7B), indicating that the green core No. 1 chip can effectively perform GWAS analysis on mung bean traits.

### Embodiment 6: Green Core No. 1 Association Analysis for Mung Bean Flowering Time

Experimental materials: 414 mung bean samples for flowering time trait association analysis; source of genotypic data: obtained from green core No. 1 chip detection; analysis model: 3VmrMLM.

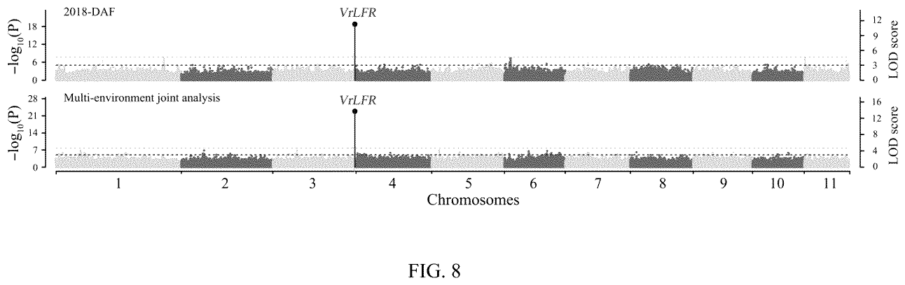

Experimental results: Using the 3VmrMLM to perform the association analysis of the flowering time trait in the tested population, the results indicate that at the level of P<1×10⁻⁷, there are 15 significant QTNs associated with flowering time. At the site Chr3-47757894 (P=5.21E-10 to 1.02E-09; LOD=11.30; r²=0.95%), there is an association with days to anthesis and flowering (DAF) (FIG. 8). There are seven genes near this site, including EVM0009174, EVM0010314, VrLFR, EVM0030971, EVM0026674, EVM0024476, and EVM0028334. Among them, VrLFR/EVM0022630 showed significant differential expression among mung bean germplasms with different flowering times.

| Gene ID | Chr | Start | End |
|---|---|---|---|
| EVM0009174 | LG03 | 47732622 | 47734578 |
| EVM0010314 | LG03 | 47738708 | 47739049 |
| EVM0022630 | LG03 | 47751486 | 47754676 |
| EVM0030971 | LG03 | 47760107 | 47761785 |
| EVM0026674 | LG03 | 47765108 | 47767726 |
| EVM0024476 | LG03 | 47769510 | 47778600 |
| EVM0028334 | LG03 | 47781660 | 47782190 |
| EVM0025581 | LG03 | 47786207 | 47793313 |

### Embodiment 7: Green Core No. 1 Association Analysis for Mung Bean Seed Weight

Experimental materials: 414 mung bean samples for seed weight trait association analysis; the source of genotypic data: obtained from green core No. 1 chip detection; and analysis model: 3VmrMLM.

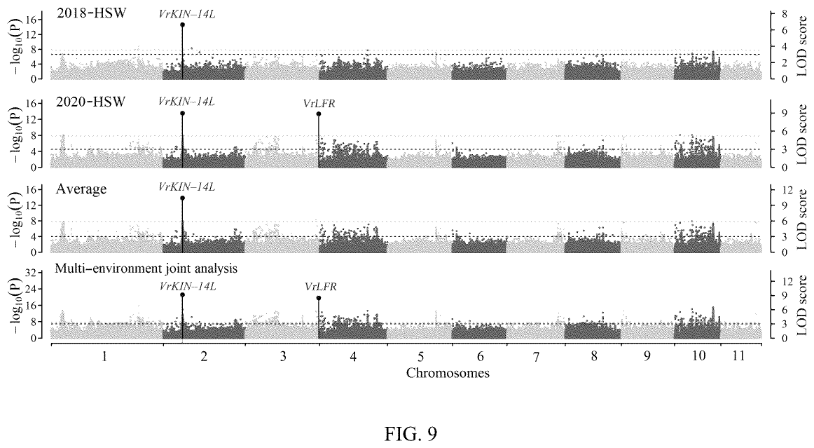

Experimental results: Using the 3VmrMLM for association analysis of the seed weight trait in the tested population, the results show that at the level of P<1×10⁻⁷, there are 19 significant QTNs significantly associated with mung bean seed weight sites. At the site Chr2-12434020 (P=2.56E-9 to 1.02E-08; LOD=6.63 to 19.53; r²=0.47% to 1.14%) (FIG. 9), there is an association with the hundred-seed weight (HSW); there is also an association with DAF. Near this site, there are four genes, including VrKIN-14L, EVM0001506, EVM0032287, and EVM0029001. Among them, VrKIN-14L/EVM0013458 shows significant differential expression between germplasms with different seed weights, and there are few research reports on related genes.

### Embodiment 8: Application of the Green Core No. 1 Chip in Genomic Selection (GS)

Experimental materials: population analysis of 892 MAGIC materials; analysis: important traits such as plant height, disease resistance, and hundred-seed weight (100-seed weight); the source of genotypic data: obtained from the green core No. 1 chip detection; and analysis models: Bayes B(BB), Bayesian least absolute shrinkage and selection operator (BL), Bayesian ridge regression (BRR), genomic best linear unbiased prediction (GB), random forest (RF), and reproducing kernel Hilbert space (RKHS) models.

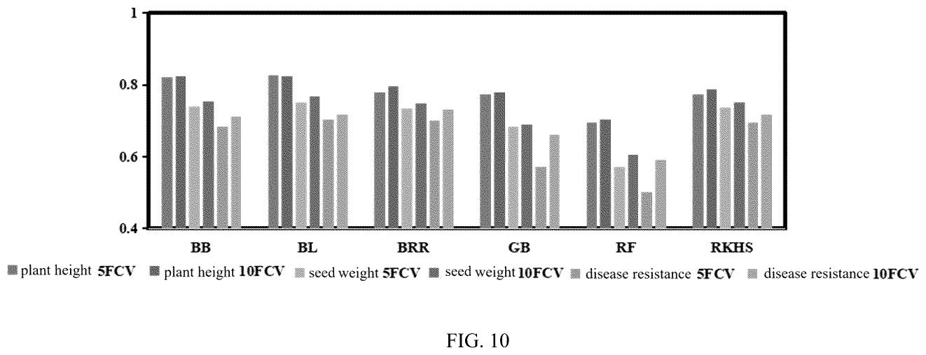

Prediction accuracy validation: 5-fold cross-validation and 10-fold cross-validation are used to compare the correlation between predicted genomic estimated breeding values (GEBV) and phenotypes. The results show that the RF model, which is constructed based on pedigree relationships, has the lowest prediction accuracy for all traits, all below 0.73. The prediction accuracy of the BL method is between 0.73 and 0.86, and the predictive performance of the BL method is superior to other methods. It can be seen that in this experiment, the relationships between materials could not improve the prediction accuracy, and the BL model, which is based on the genotypic data, performs better. Taking the BL as an example, the prediction accuracy for the plant height is the highest, reaching 0.83, and the accuracies for the hundred-seed weight and the disease resistance also reach above 0.75 (Table 4, FIG. 10). The selected marker sites have a significant advantage in prediction accuracy. Machine learning methods may not be prominent due to the size of the sample quantity. In summary, the green core No. 1 chip can be used for molecular breeding analysis and prediction in mung beans.

TABLE 4
Results of prediction accuracy validation

| | Grouping | Bayesian method | | | Machine learning method | | |
|---|---|---|---|---|---|---|---|
| Trait | scheme | BB | BL | BRR | GB | RF | RKHS |
| Plant | 5FCV | 0.855 ± 0.006 | 0.861 ± 0.006 | 0.811 ± 0.008 | 0.807 ± 0.012 | 0.723 ± 0.007 | 0.805 ± 0.008 |
| height | 10FCV | 0.858 ± 0.011 | 0.859 ± 0.001 | 0.829 ± 0.002 | 0.811 ± 0.015 | 0.734 ± 0.020 | 0.820 ± 0.004 |
| Seed | 5FCV | 0.771 ± 0.010 | 0.784 ± 0.008 | 0.765 ± 0.007 | 0.713 ± 0.021 | 0.597 ± 0.023 | 0.768 ± 0.012 |
| weight | 10FCV | 0.786 ± 0.014 | 0.801 ± 0.009 | 0.780 ± 0.012 | 0.718 ± 0.010 | 0.632 ± 0.019 | 0.783 ± 0.015 |
| Disease | 5FCV | 0.714 ± 0.014 | 0.732 ± 0.009 | 0.731 ± 0.010 | 0.595 ± 0.011 | 0.523 ± 0.016 | 0.723 ± 0.012 |
| resistance | 10FCV | 0.741 ± 0.019 | 0.749 ± 0.015 | 0.763 ± 0.011 | 0.688 ± 0.012 | 0.617 ± 0.025 | 0.749 ± 0.014 |

It should be noted that the terms herein “including,” “containing” or any other variation thereof are intended to encompass non-exclusive inclusion, such that a process, method, article, or apparatus that includes a series of elements not only includes those elements, but also includes other elements not explicitly listed, or also includes elements inherent to such process, method, article, or apparatus. Without further limitations, the element limited by the statement “including one . . . ” does not exclude the existence of other identical elements in the process, method, article, or device that includes that element.

The above embodiments of the disclosure are numbered for description only and do not represent the advantages or disadvantages of the embodiments.

The above embodiments are only used to illustrate the technical solution of the disclosure and not to limit it. Although the disclosure has been described in detail with reference to the preferred embodiments, those skilled in the art should understand that the technical solution of the disclosure can be modified or equivalently replaced without departing from the purpose and scope of the technical solution of the disclosure, and all of them should be included in the scope of the claims of the disclosure.

1. (canceled)

2. A mung bean SNP chip, comprising:
(1) a probe configured to detect a mung bean SNP molecular marker combination; or
(2) a primer and the probe configured to detect the mung bean SNP molecular marker combination;
wherein the mung bean SNP molecular marker combination comprises 20526 SNP molecular markers as shown in Table 2, a position and allelic information of each of the SNP molecular markers are in a format of chromosome: physical position: reference genotype/variant allele genotype;
wherein the SNP molecular markers are based on a reference genome Wm82. a2. v1.

3. A kit, comprising:
(1) a probe configured to detect a mung bean SNP molecular marker combination: or
(2) a primer and the probe configured to detect the mung bean SNP molecular marker combination;
wherein the mung bean SNP molecular marker combination comprises 20526 SNP molecular markers as shown in Table 2, a position and allelic information of each of the SNP molecular markers are in a format of chromosome: physical position: reference genotype/variant allele genotype;
wherein the SNP molecular markers are based on a reference genome Wm82. a2. v1.

4. An application of the mung bean SNP chip as claimed in claim 2, comprising: applying the mung bean SNP chip in mung bean variety identification.

5. An application of the mung bean SNP chip as claimed in claim 2, comprising: applying the mung bean SNP chip in detecting a mung bean breeding material resistant to yellow mosaic disease.

6. An application of the mung bean SNP chip as claimed in claim 2, comprising: applying the mung bean SNP chip in mung bean genome-wide association analysis.

7. An application of the mung bean SNP chip as claimed in claim 2, comprising: applying the mung bean SNP chip in molecular marker assisted breeding.

8. An application of the mung bean SNP chip as claimed in claim 2, comprising: applying the mung bean SNP chip in deoxyribonucleic acid (DNA) fingerprint analysis of mung bean germplasm resources.

9. An application of the mung bean SNP chip as claimed in claim 2, comprising: applying the mung bean SNP chip in mung bean genomic selection breeding.

10. An application of the mung bean SNP chip as claimed in claim 2, comprising: applying the mung bean SNP chip in one of the following applications:
(1) mung bean parentage identification,
(2) mung bean population structure analysis,
(3) mung bean genetic diversity analysis,
(4) mung bean variety authenticity testing, and
(5) mung bean cultivar genotyping identification.
